(19) United States
(12) Patent Application Publication
(10) Pub. No.: US 2025/0186493 A1
(43) Pub. Date: Jun. 12, 2025

(54) ARTIFICIAL ANTIGEN-SPECIFIC IMMUNOREGULATORY T (AIRT) CELLS

(71) Applicants: Seattle Children's Hospital (dba Seattle Children's Research Institute), Seattle, WA (US); Benaroya Research Institute at Virginia Mason, Seattle, WA (US)

(72) Inventors: Jane Buckner, Seattle, WA (US); David J. Rawlings, Seattle, WA (US); Peter J. Cook, Seattle, WA (US); Soo Jung Yang, Seattle, WA (US)

(21) Appl. No.: 18/268,885

(22) PCT Filed: Dec. 21, 2021
(86) PCT No.: PCT/US2021/064561

§ 371 Date: Jun. 21, 2023

Related U.S. Application Data
(60) Provisional application No. 63/129,288, filed on Dec. 22, 2020.
(60) Provisional application No. 63/155,486, filed on Mar. 2, 2021.
(60) Provisional application No. 63/179,068, filed on Apr. 23, 2021.
(60) Provisional application No. 63/274,375, filed on Nov. 1, 2021.

(51) Int. Cl. A61K 35/17; A61K 40/11; A61K 40/22; A61K 40/31; A61K 40/32; A61K 40/41; A61P 37/06; C07K 14/715; C07K 14/725; C12N 5/0783; C12N 9/22; C12N 15/11

(52) U.S. Cl. CPC A61K 35/17; A61K 40/11; A61K 40/22; A61K 40/31; A61K 40/32; A61K 40/416; A61P 37/06; C07K 14/7051; C07K 14/7155; C12N 5/0637; C12N 9/22; C12N 15/111; A61K 2239/21; C07K 2319/03; C07K 2319/70; C12N 2310/20; C12N 2510/00

(57) ABSTRACT

Some embodiments of the methods and compositions provided herein relate to efficient editing of more than one genetic locus in a cell using a chemical-inducible signaling complex (CISC) system. Some embodiments include use of such systems to edit a FOXP3 locus gene and TRAC locus in a Treg cell. More embodiments herein relate to use of gene-edited Treg cells to suppress activation and/or proliferation of certain populations of cells.

### RELATED APPLICATIONS

This application claims priority to U.S. Prov. No. 63/274,375 Nov. 1, 2021; U.S. Prov. No. 63/179,068 filed Apr. 23, 2021; U.S. Prov. No. 63/155,486 filed Mar. 2, 2021; and U.S. Prov. No. 63/129,288 filed Dec. 22, 2020, each entitled “ARTIFICIAL ANTIGEN-SPECIFIC IMMUNOREGULATORY T (AIRT) CELLS,” each of which is expressly incorporated herein by reference in its entirety.

### REFERENCE TO SEQUENCE LISTING

The present application is being filed along with a Sequence Listing in electronic format. The Sequence Listing is provided as a file entitled SCRI351WOSEQLIST, created Dec. 20, 2021, which is approximately 746,677 bytes in size. The information in the electronic format of the Sequence Listing is incorporated herein by reference in its entirety.

### FIELD OF THE INVENTION

Some embodiments of the methods and compositions provided herein relate to efficient editing of more than one genetic locus in a cell using a chemical-inducible signaling complex (CISC) system. Some embodiments include use of such systems to edit a FOXP3 locus gene and TRAC locus in a Treg cell. More embodiments relate to use of gene-edited Treg cells to suppress activation and/or proliferation of certain populations of cells.

### BACKGROUND OF THE INVENTION

Autoimmune diseases, such as type 1 diabetes mellitus, multiple sclerosis, myocarditis, rheumatoid arthritis (RA), and systemic lupus erythematosus (SLE, or “lupus”), are chronic, often life-threatening conditions that result from alterations in immunological self-tolerance, leading to aberrant immune activity and end-organ pathology. Inappropriate and deleterious dysregulation of immune tolerance can also contribute undesirably to pathologies associated with allergy, asthma, transplant rejection, and/or graft-versus-host disease (GVHD). The role of specialized antigen-recognizing thymic-derived T lymphocytes known as regulatory T cells (Treg, also referred to as suppressor T cells) in the maintenance of immune tolerance and prevention of autoimmunity is well established, and multiple autoimmune conditions are characterized by dysfunctional or dysregulated Treg compartments.

As a potential therapy for autoimmune disease, adoptive transfer to an afflicted subject of functional Treg selected for their immunosuppressive ability has been explored in mouse models and early phase clinical trials. However, a lack of autoantigen specificity of such Treg cells, and uncontrolled cell plasticity (e.g., conversion from immunosuppressive negative regulator of immunity to pro-inflammatory effector-like phenotype) resulting in the loss of immunosuppressive Treg activity, comprise two major limitations for the effective and sustained therapeutic benefit of such Treg adoptive transfer. It is believed that the use of immunosuppressive Treg selected to respond antigen-specifically to disease-associated autoantigens would lead to a safer, more effective adoptive transfer strategy than simple transfer of polyspecific Treg. In this respect, following infusion into a subject, the autoantigen-specific Treg cells would be expected to home specifically to tissue sites where autoimmune activity is manifest and, importantly, would mediate immune suppression specifically in response to the autoantigens that drive autoimmune disease pathogenesis. In support of this concept, studies in mice have shown that antigen-specific Tregs are more efficacious than polyclonal Tregs in murine models of autoimmune disease. (Duggleby et al., 2018 Front. Immunol. 9:252; Tang et al 2004 J Exp Med. 199(11):1455-1465; Tarbell et al 2004 J Exp Med 199:1467-1477.)

Therapeutic applications of adoptively transferred antigen-specific Treg or even of polyclonal Treg to treat autoimmune disease have, however, been limited, inter alia, by difficulties encountered in the course of isolating sufficient quantities of rare, antigen-specific Treg cells from a natural source, such as blood, and lymph, and by the overall scarcity of natural Treg in the peripheral blood, for example, approximately 14% of peripheral blood mononuclear cells include natural Treg. Development of Treg adoptive transfer therapies has also been hindered by challenges associated with expanding Treg populations to therapeutic numbers ex vivo while maintaining their immunosuppressive function, with the poor ability of adoptively transferred Treg cells to persist in an adoptive host and to proliferate after re-infusion. Also problematic has been Treg plasticity, such as conversion from immunosuppressive negative regulator of immunity to pro-inflammatory effector-like phenotype, in inflammatory settings in vivo. (Singer et al., 2014 Front. Immunol. 5:Art. 46; Trzonkowski et al., 2015 Sci. Translat. Med. 7(304):ps18 Romano et al., 2016 Transplant. Internatl. 30:745, McGovern et al., 2017 Front. Immunol. 8:Art. 1517).

No prior approaches provide bulk populations of stable Treg cells with suppressive activity having a desired antigen specificity, such as specificity for an antigen involved in the pathogenesis of a condition where antigen-specific immunosuppression would be beneficial, for instance, autoimmune disease, allergy, and/or other inflammatory conditions.

Accordingly, there remains a need for stable, antigen-specific immunoregulatory cells that maintain antigen-specific immunosuppressive capability in vitro and in vivo without exhibiting plasticity, as may be usefully administered to subjects in need of antigen-specific immunosuppression by adoptive transfer immunotherapy.

### SUMMARY OF THE INVENTION

Some embodiments include a gene editing chemical-inducible signaling complex (CISC) system comprising: a first polynucleotide encoding a first promoter and a nucleic acid encoding a first CISC component comprising a first extracellular binding domain, a transmembrane domain, and a first signaling domain, wherein the first promoter is proximal to the nucleic acid encoding a first CISC component; and a second polynucleotide encoding a second promoter and a nucleic acid encoding a second CISC component comprising a second extracellular binding domain, a transmembrane domain, and a second signaling domain; wherein the first CISC component and the second CISC component are configured such that when expressed in a cell, they are capable of dimerizing in the presence of rapamycin or a rapalog to generate a signaling-competent CISC. In some embodiments, a second promoter is proximal to the nucleic acid encoding a second CISC component.

In some embodiments, a 3′ end of the first promoter is within 500 consecutive nucleotides from a 5′ end of the nucleic acid encoding a first CISC component. In some embodiments, a 3′ end of the first promoter is within 100 consecutive nucleotides from a 5′ end of the nucleic acid encoding a first CISC component.

In some embodiments, the first polynucleotide is configured for integration into a first target locus of a genome, and the second polynucleotide is configured for integration into a second target locus of the genome. In some embodiments, the first target locus is selected from a TRAC locus or a FOXP3 locus; and the second target locus is selected from a TRAC locus or a FOXP3 locus.

In some embodiments, the first extracellular binding domain comprises an FK506 binding protein (FKBP)-rapamycin binding (FRB) domain; and the second extracellular binding domain comprises an FKBP domain.

In some embodiments, the first signaling domain comprises an IL-2 receptor subunit beta (IL2Rβ) domain or functional derivative thereof; and the second signaling domain comprises an IL-2 receptor subunit gamma (IL2Rβγ) domain or functional derivative thereof. In some embodiments, the IL2Rβ domain comprises a truncated IL2Rβ domain.

In some embodiments, the first and/or second promoter comprises a constitutive promoter. In some embodiments, the first and/or second promoter comprises a MND promoter.

In some embodiments, a first vector comprises the first polynucleotide, and a second vector comprises the second polynucleotide. In some embodiments, the first vector and/or the second vector comprises a viral vector. In some embodiments, the first vector and/or the second vector comprises a lentiviral, an adenoviral, or an adeno-associated viral (AAV) vector.

In some embodiments, the first polynucleotide and/or the second polynucleotide comprises a nucleic acid encoding a naked FRB domain, wherein the nucleic acid encoding a naked FRB domain lacks a nucleic acid encoding an endoplasmic reticulum localization signal polypeptide.

In some embodiments, the first polynucleotide and/or the second polynucleotide comprises a nucleic acid encoding a payload. In some embodiments, the first polynucleotide and/or the second polynucleotide comprises a nucleic acid encoding a self-cleaving polypeptide, wherein the nucleic acid encoding a self-cleaving polypeptide is 5′ of the nucleic acid encoding a payload. In some embodiments, the self-cleaving polypeptide is selected from the group consisting of P2A, T2A, E2A, and F2A. In some embodiments, payload comprises a T cell receptor (TCR), chimeric antigen receptor (CAR), or functional fragment thereof. In some embodiments, the TCR or functional fragment thereof comprises the polypeptide sequence of any one of SEQ ID NOs 1377-1390.

In some embodiments, the first polynucleotide and/or the second polynucleotide is configured for integration into a target genomic locus by homology directed repair (HDR) or by non-homologous end joining (NHEJ).

Some embodiments also include a guide RNA (gRNA) and a DNA endonuclease. In some embodiments, the DNA endonuclease comprises a Cas9 endonuclease.

In some embodiments, the rapalog is selected from the group consisting of everolimus, CCI-779, C20-methallylrapamycin, C16-(S)-3-methylindolerapamycin, C16-iRap, C16-(S)-7-methylindolerapamycin, AP21967, C16-(S)Butylsulfonamidorapamycin, AP23050, sodium mycophenolic acid, benidipine hydrochloride, AP1903, and AP23573, and a metabolite or derivative thereof.

Some embodiments include a cell comprising any one of the foregoing systems or systems described herein. In some embodiments, provided herein is a cell (e.g., a human cell) in which the endogenous TCR-encoding locus (e.g., TRAC gene/locus) has been edited. In some embodiments, a TRAC gene in a cell is edited by promoter capture (e.g., the method depicted in FIGS. 54, 68, and 70). In some embodiments, a TRAC gene in a cell is edited by knocking in a full TCR (e.g., an islet TCR) with a promoter (e.g., MND promoter). See FIG. 67 as an example of such a method. In some embodiments, a TRAC gene in a cell is edited by hijacking the TRAC gene with a promoter (e.g., MND promoter) as shown in FIG. 164. In some embodiments, a cell as provided herein is a human cell. In some embodiments, a cell is a lymphocyte (e.g., a NK1.1+, CD3+, CD4+ or CD8+ cell). In some embodiments, the cell is a T cell, a precursor T cell, or a hematopoietic stem cell. In some embodiments, the cell is an NK-T cell (e.g., a FOXP3− NK-T cell or a FOXP3+ NK-T cell). In some embodiments, the cell is a regulatory B (Breg) cell (e.g., a FOXP3− B cell or a FOXP3+ B cell). In some embodiments, the cell is a CD4+ T cell (e.g., a FOXP3−CD4+ or a FOXP3+CD4+ T cell) or a CD8+ T cell (e.g., a FOXP3−CD8+ or a FOXP3+CD8+ T cell). In some embodiments, the cell is a CD25− T cell. In some embodiments, the cell is a regulatory T (Treg) cell. Non-limiting examples of Treg cells are Tr1, Th3, CD8+CD28−, and Qa-1 restricted T cells. In some embodiments, the cell is a T regulatory type 1 (Tr1) cell. In some embodiments, the Treg cell is a FOXP3+ Treg cell. In some embodiments, the Treg cell expresses CTLA-4, LAG-3, CD25, CD39, neuropilin-1, galectin-1, and/or IL-2Ra on its surface. In some embodiments, a cell as provided herein is an engineered cell. In some embodiments, an engineered cell is a cell in which one or more genes/loci are manipulated or edited (e.g., to stabilize expression of one or more genes). In some embodiments, an engineered cell comprises editing of the Foxp3 gene/locus, e.g., by inserting a promoter (in some embodiments, downstream of one or more regulatory elements like the TSDR, and/or upstream from the first coding exon or first codon). In some embodiments, the cell is ex vivo. In some embodiments, a cell is in vivo. In some embodiments, the cell is a human cell. In some embodiments, a cell as described here in is isolated from a biological sample. A biological sample may be a sample from a subject (e.g., a human subject) or a composition produced in a lab (e.g., a culture of cells). A biological sample obtained from a subject make be a liquid sample (e.g., blood or a fraction thereof, a bronchial lavage, cerebrospinal fluid, or urine), or a solid sample (e.g., a piece of tissue). In some embodiments, the cell is obtained from peripheral blood. In some embodiments, the cell is obtained from umbilical cord blood.

Some embodiments include a pharmaceutical composition comprising any one of the foregoing cells and a pharmaceutically acceptable excipient.

Some embodiments include a method of editing a cell, comprising obtaining any of the foregoing systems; introducing the first polynucleotide and the second polynucleotide into a cell to obtain a transduced cell; and culturing the transduced cell. Some embodiments also include contacting the transduced cell with the rapamycin or rapalog. In some embodiments, contacting of a cell with rapamycin or a rapalog is performed ex vivo, e.g., to select cells. In some embodiments, contacting of a cell with rapamycin or a rapalog is performed in vivo, e.g., to activate or maintain activity of a cell via the CISC machinery in the cell; thus resulting in a stable suppressive phenotype of function (e.g., by maintaining expression of I1-2, or STAT5) or by maintaining a high number of active suppressive cells (e.g., stabilizing a suppressive phenotype). In some embodiments, contacting the cell with rapamycin or a rapalog in vivo maintains the number of suppressive cells at 50% or more, 60% or more, 70% or more, 80% or more, or 90% or more of the number of suppressive cells that were administered. In some embodiments, contacting the cell with rapamycin or a rapalog in vivo maintains expression of one or more cytokines at a level that is 50% or more, 60% or more, 70% or more, 80% or more, or 90% or more of the level of cytokine expression level observed prior to administration. In some embodiments, cells comprising CISC machinery as described herein and rapamycin or a rapalog are administered to a subject simultaneously. In some embodiments, cells comprising CISC machinery as described herein and rapamycin or a rapalog are administered to a subject sequentially.

Some embodiments include a method of suppressing proliferation of a population of cells, comprising contacting the population of cells with a genetically modified Treg cell, such as a CD4+ or CD8+ Treg cell; wherein the population of cells comprise an endogenous T cell receptor (TCR) specific for a first epitope of an antigen, and wherein the Treg cell comprises an exogenous TCR specific for a second epitope of the antigen.

Some embodiments include a method of treating, ameliorating or inhibiting a disorder in a subject comprising: administering a genetically modified Treg cell, such as a CD4+ or CD8+ Treg cell, to the subject to suppress proliferation of a population of cells; wherein the population of cells comprise an endogenous T cell receptor (TCR) specific for a first epitope of an antigen, and wherein the Treg cell comprises an exogenous TCR specific for a second epitope of the antigen.

In some embodiments, the disorder comprises an autoimmune disorder. In some embodiments, the subject is mammalian. In some embodiments, the subject is human.

In some embodiments, the exogenous TCR has an increased avidity for the antigen compared to an additional TCR specific for the antigen.

In some embodiments, the exogenous TCR has a reduced avidity for the antigen compared to an additional TCR specific for the antigen.

In some embodiments, the population of cells comprises CD4+ CD25− T cells. In some embodiments, the population of cells comprises polyclonal T cells.

In some embodiments, the exogenous TCR is specific for a type I diabetes antigen. In some embodiments, the exogenous TCR is specific for a type I diabetes antigen selected from IGRP, GAD65, and PPI. In some embodiments, the exogenous TCR is selected from T1D2, T1D4, T1D5-1, T1D5-2, 4.13, GAD113, and PPI76. In some embodiments, the exogenous TCR comprises T1D5-2.

In some embodiments, the population of cells are contacted with the genetically modified Treg cell in the presence of an antigen presenting cell and the antigen.

In some embodiments, the Treg cell is obtained by introducing into a cell a vector comprising a nucleic acid encoding the exogenous TCR.

In some embodiments, the Treg cell is mammalian. In some embodiments, the Treg cell is human.

Some embodiments of the methods and compositions provided herein include an artificial cell (e.g., CD4+CD25+ antigen-specific immunoregulatory T (airT) cell, also referred to as an engineered regulatory-like T (EngTreg) or edited regulatory-like T (edTreg) cell), comprising: (a) an artificial modification of a forkhead box protein 3/winged helix transcription factor (FOXP3) gene, wherein the modified gene constitutively expresses a FOXP3 gene product at a FOXP3 expression level that is equal to or greater than the FOXP3 expression level of a naturally occurring regulatory T (Treg) cell; and (b) at least one transduced polynucleotide encoding an antigen-specific T cell receptor (TCR) polypeptide (e.g., in a TRAC locus/gene). FIGS. 33 and 54 provides an example of such a cell. FIGS. 54, 67-68, 70, and 164 provide examples methods by which a TCR polypeptide may be transduced.

In some embodiments there is provided an artificial CD4+CD25+ antigen-specific immunoregulatory T (airT) cell obtained by (i) artificial modification of a forkhead box protein 3/winged helix transcription factor (FOXP3) gene in a CD4+CD25⁻ T cell, wherein the artificial modification causes the airT cell to constitutively express a FOXP3 gene product at a FOXP3 expression level that is equal to or greater than the FOXP3 expression level of a naturally occurring regulatory T (Treg) cell; and (ii) insertion of at least one transduced polynucleotide encoding an antigen-specific T cell receptor (TCR) or chimeric antigen receptor (CAR) polypeptide. In some embodiments there is provided an artificial antigen-specific immunoregulatory T (airT) cell (e.g., CD4+ or CD8+) obtained by (i) artificial modification of a forkhead box protein 3/winged helix transcription factor (FOXP3) gene in a CD25⁻ T cell, wherein the artificial modification causes the airT cell to constitutively express a FOXP3 gene product at a FOXP3 expression level that is equal to or greater than the FOXP3 expression level of a naturally occurring regulatory T (Treg) cell; and (ii) insertion of at least one transduced polynucleotide encoding an antigen-specific T cell receptor (TCR) or chimeric antigen receptor (CAR) polypeptide.

In some embodiments, the FOXP3 gene is present in a FOXP3 gene locus comprising a regulatory element that is capable of regulating expression of FOXP3 in a naturally occurring Treg cell. In some embodiments the FOXP3 gene is present in a FOXP3 gene locus comprising an intronic regulatory T cell (Treg)-specific demethylation region (TSDR) having a plurality of cytosine-guanine (CG) dinucleotides, wherein each CG dinucleotide comprises a methylated cytosine (C) nucleotide at a nucleotide position that comprises a demethylated C nucleotide in a naturally occurring Treg cell. In some embodiments at least 80%, 85%, 90%, 95%, 96%, 97%, 98%, or 99% of the TSDR C nucleotides at nucleotide positions that comprise a demethylated C nucleotide in a naturally occurring Treg cell are methylated. In some embodiments the FOXP3 gene product is expressed at a level sufficient for the airT cell to maintain a CD4+CD25+ phenotype for at least 21 days in vitro. In some embodiments the FOXP3 gene product is expressed at a level sufficient for the airT cell to maintain a CD4+CD25+ phenotype for at least 60 days in vivo following adoptive transfer to an immunocompatible mammalian host in need of antigen-specific immunosuppression. In some embodiments the cell comprises a phenotype selected from one or more of: (i) HeliosLo, (ii) CD152+, (iii) CD127⁻, or (iv) ICOS+. In some embodiments the artificial modification comprises a knockout of a native FOXP3 gene locus in the cell.

In some embodiments the artificial modification comprises an inserted nucleic acid molecule comprising a heterologous promoter at a native FOXP3 gene locus of the cell, wherein the promoter is positioned in the FOXP3 gene such that it is capable of promoting transcription of an endogenous FOXP3-encoding nucleotide sequence of the FOXP3 gene locus. In some embodiments, the heterologous promoter is a constitutive promoter, which promotes transcription of an operably linked sequence (e.g., a FOXP3 gene) at a consistent rate. Constitutive promoters may be strong promoters, which promote transcription at a higher rate than an endogenous promoter, or weak promoters, which promote transcription at a lower rate than a strong or endogenous promoter. In some embodiments, the constitutive promoter is a strong promoter. In some embodiments, the constitute promoter is a weak promoter. In some embodiments, the strong promoter is an MND promoter. In some embodiments, the constitutive promoter is a PGK promoter, MND promoter, or EF-1a promoter. In some embodiments, the heterologous promoter is an inducible promoter. Inducible promoters promote transcription of an operably linked sequence in response to the presence of an activating signal, or the absence of a repressor signal. In some embodiments, the inducible promoter is inducible by a drug or steroid.

In some embodiments the inserted nucleic acid molecule further comprises a nucleic acid sequence encoding a first chemically inducible signaling complex (CISC) component capable of specifically binding to a CISC inducer molecule. In some embodiments the transduced polynucleotide encoding the TCR polypeptide further comprises a nucleic acid sequence encoding a second chemically inducible signaling complex (CISC) component that is different from the first CISC component and is capable of specifically binding to the CISC inducer molecule. In some embodiments the nucleic acid sequence encoding the first CISC component and the nucleic acid sequence encoding the second CISC component further comprises a nucleic acid sequence encoding a third CISC component that is different from the first and second CISC components and is capable of specifically binding to the CISC inducer molecule. In some embodiments, the third CISC component is soluble and does not comprise a transmembrane domain or an extracellular domain. In some embodiments, the soluble third CISC component does not comprise a secretory peptide and is localized in the cytoplasm of the cell. In some embodiments the nucleic acid molecule comprising the constitutively active promoter is inserted downstream of an intronic regulatory T cell (Treg)-specific demethylation region (TSDR) in the native FOXP3 gene locus. In some embodiments the constitutively active promoter is an MND promoter. In some embodiments the artificial modification comprises an inserted nucleic acid molecule comprising an exogenous FOXP3-encoding polynucleotide operably linked to a constitutively active promoter at a native FOXP3 gene locus of the cell. In some embodiments the inserted nucleic acid molecule comprising the exogenous FOXP3-encoding polynucleotide operably linked to the constitutively active promoter further comprises a nucleic acid sequence encoding a first chemically inducible signaling complex (CISC) component capable of specifically binding to a CISC inducer molecule. In some embodiments the transduced polynucleotide encoding a gene (e.g., a FOXP3 or TCR polypeptide) further comprises a nucleic acid sequence encoding a second chemically inducible signaling complex (CISC) component that is different from the first CISC component and is capable of specifically binding to the CISC inducer molecule. In some embodiments at least one of the nucleic acid sequence encoding the first CISC component and the nucleic acid sequence encoding the second CISC component further comprises a nucleic acid sequence encoding a third CISC component that is different from the first and second CISC components and is capable of specifically binding to the CISC inducer molecule. In some embodiments, the third CISC component is soluble and does not comprise a transmembrane domain or an extracellular domain. In some embodiments, the soluble third CISC component does not comprise a secretory peptide and is localized in the cytoplasm of the cell. FIGS. 33 and 54 depict an example.

In some embodiments the nucleic acid molecule comprising the exogenous FOXP3-encoding polynucleotide operably linked to the constitutively active promoter is inserted downstream of an intronic regulatory T cell (Treg)-specific demethylation region (TSDR) in the native FOXP3 gene locus. In some embodiments the constitutively active promoter is an MND promoter.

In some embodiments the artificial modification comprises an insertion of a nucleic acid molecule comprising an exogenous FOXP3-encoding polynucleotide operably linked to a constitutively active promoter at a chromosomal site other than a native FOXP3 gene locus of the cell. In some embodiments at least one native T cell receptor (TCR) gene locus of the airT cell is knocked out or inactivated and replaced with the at least one transduced polynucleotide encoding an antigen-specific T cell receptor (TCR) polypeptide. In some embodiments, a promoter capture method is used in which a native/naturally-occurring/endogenous promoter is relied upon for control of inserted TCR-encoding nucleic acid, which may be followed by nucleic acid encoding one or more CISC components as described herein (see e.g., FIGS. 54, 68, 70). In some embodiments such an endogenous promoter capture method comprises in-frame knock-in to TRAC exon 1 to drive expression of a TCR relying on endogenous TRAC promoter. In some embodiments, a TCR knock-in strategy is employed and comprises knock-in of a promoter and TCR-encoding nucleic acid, which may be followed by nucleic acid encoding one or more CISC components as described herein (see e.g., FIG. 67). In some embodiments, the native/naturally-occurring/endogenous TCR gene or fragments thereof are hijacked by insertion of a promoter upstream from the native/naturally-occurring/endogenous and optionally upstream from nucleic acid encoding one or more CISC components as described herein (see e.g., FIG. 164). The TRAC gene/locus may be, in some embodiments, be combined with insertion of a promoter downstream from the TSDR in the Foxp3 gene/locus.

In some embodiments, each inserted nucleic acid encodes either a first or second CISC component, such that cells comprising both inserted nucleic acids express both the first and second CISC components, while cells comprising only one inserted nucleic acid do not express both first and second CISC components. In such embodiments, cells comprising both inserted nucleic acids can be selected by providing a CISC inducer molecule, which promotes dimerization of the first and second CISC components, resulting in transduction of a proliferative signal. When such inserted nucleic acids comprise other genetic modifications, such as an exogenous TCR or CAR, or insertion of a heterologous promoter to drive expression of an endogenous FOXP3 gene independently of regulation by endogenous regulatory elements, cells comprising said genetic modifications can be selected on the basis of expression of both CISC components. Thus, a cell comprising a first inserted nucleic acid molecule encoding a first CISC component and a TCR or CAR, and a second inserted nucleic acid molecule encoding a second CISC component and comprising a heterologous promoter operably linked to an endogenous FOXP3 gene, can be selected by providing a CISC inducer molecule. Therefore, providing the CISC inducer molecule can select for airT cells with a desired antigen specificity and expressing FOXP3 independently of endogenous regulatory mechanisms, such as suppression by a methylated TSDR.

Any known method of gene editing may be used for insertion of nucleotide sequences or modification of genomic loci. Non-limiting gene editing methods include zinc finger nuclease (ZFN)-mediated gene editing, transcription activator-like effector nuclease (TALEN)-mediated gene editing, meganuclease-mediated gene editing, transposon-mediated gene editing, serine integrase-mediated gene editing, lentivirus-mediated gene editing, RNA-guided nuclease (RGN)-mediated gene editing, CRISPR/Cas-mediated gene editing, homologous recombination-mediated gene editing, and combinations thereof. ZFNs are restriction enzymes that are made by fusing a zinc finger DNA-binding domain to a DNA-cleavage domain. Thus, ZFNs bind to specific DNA sequences, based on their specificity, and cleave DNA following binding. TALENs are restriction enzymes that contain a TAL effector domain, which bind to specific DNA sequences and can be modified to recognize a desired DNA sequence, and a DNA cleavage domain of a FokI endonuclease. Thus, TALENs bind to specific DNA sequences, based on their specificity, and cleave DNA after binding to their target sequence. Meganucleases are targeted nucleases derived from homing endonucleases, such as I-CreI and I-SceI, that bind to specific DNA sequences, based on their specificity, and cleave DNA following binding. Transposons are chromosomal elements that are capable of undergoing transposition, in which they can be excised from one position on a chromosome and introduced at another position. Transposases mediate the integration of transposons into a chromosome. In transposon-mediated gene editing, a desired sequence is inserted into a transposon, and the transposon containing the desired sequence is introduced to a cell, along with transposase or a nucleic acid encoding the transposase. The action of transposase mediates insertion of the transposon containing the desired sequence into the chromosome. See, e.g., Ivics and Izsvák. Curr Gene Ther. 2006. 6(5):593-607. RNA-guided nucleases are nucleases that bind to a conserved nucleotide sequence on RNA guide, with the RNA guide having a targeting nucleotide sequence that is complementary to a target nucleotide sequence on a nucleic acid to be modified (e.g., a chromosome). The targeting nucleotide sequence thus directs the RNA-guided nuclease to a nucleic acid comprising the target sequence, and the RNA-guided nuclease cleaves DNA after the RNA guide binds to the target sequence. Non-limiting examples of RNA-guided nucleases include those provided in U.S. Pat. No. 11,162,114, which is incorporated by reference herein in its entirety. Other examples of RNA-guided nucleases include CRISPR-Cas-associated nucleases.

In some embodiments the at least one native TCR gene locus that is knocked out or inactivated is a native TCR alpha chain (TRAC) locus. In some embodiments the inserted nucleic acid molecule comprising the exogenous FOXP3-encoding polynucleotide operably linked to the constitutively active promoter further comprises a nucleic acid sequence encoding a first chemically inducible signaling complex (CISC) component capable of specifically binding to a CISC inducer molecule. In some embodiments the transduced polynucleotide encoding the TCR polypeptide further comprises a nucleic acid sequence encoding a second chemically inducible signaling complex (CISC) component that is different from the first CISC component and is capable of specifically binding to the CISC inducer molecule. In some embodiments at least one of the nucleic acid sequences encoding the first CISC component and the nucleic acid sequence encoding the second CISC component further comprises a nucleic acid sequence encoding a third CISC component that is different from the first and second CISC components and is capable of specifically binding to the CISC inducer molecule. In some embodiments, the third CISC component is soluble and does not comprise a transmembrane domain or an extracellular domain. In some embodiments, the soluble third CISC component does not comprise a secretory peptide and is localized in the cytoplasm of the cell. In some embodiments the constitutively active promoter is an MND promoter. In some embodiments the chromosomal site that is other than a native FOXP3 gene locus, and at which is inserted the nucleic acid molecule comprising the exogenous FOXP3-encoding polynucleotide operably linked to the constitutively active promoter, is within a T cell receptor alpha chain (TRAC) locus of the cell.

In some embodiments in the airT cell at least one native T cell receptor (TCR) gene locus is knocked out or inactivated and replaced with the at least one transduced polynucleotide encoding an antigen-specific T cell receptor (TCR) polypeptide. In some embodiments the at least one native TCR gene locus that is knocked out is a native TCR alpha chain (TRAC) locus.

In some embodiments there is provided an artificial CD4+CD25+ antigen-specific immunoregulatory T (airT) cell, comprising: (a) a transduced nucleic acid sequence encoding an exogenous forkhead box protein 3/winged helix transcription factor (FOXP3) gene product, wherein the cell constitutively expresses the FOXP3 gene product at a FOXP3 expression level that is equal to or greater than the FOXP3 expression level of a naturally occurring regulatory T (Treg) cell; and (b) at least one transduced polynucleotide encoding an exogenous antigen-specific T cell receptor (TCR) polypeptide; wherein the transduced nucleic acid sequence encoding the exogenous FOXP3 gene product further comprises a nucleic acid sequence encoding a first chemically inducible signaling complex (CISC) component capable of specifically binding to a CISC inducer molecule; and wherein the transduced nucleic acid sequence encoding the exogenous TCR gene product further comprises a nucleic acid sequence encoding a second chemically inducible signaling complex (CISC) component that is different from the first CISC component and is capable of specifically binding to the CISC inducer molecule.

In some embodiments there is provided an artificial CD4+CD25+ antigen-specific immunoregulatory T (airT) cell, comprising: (a) a native FOXP3 gene locus that has been knocked out or inactivated, and into which FOXP3 locus has been inserted, by homology-directed repair, either: (i) a nucleic acid molecule comprising a constitutively active promoter that is capable of promoting transcription of an endogenous FOXP3-encoding nucleotide sequence of the FOXP3 gene, or (ii) a nucleic acid molecule comprising a constitutively active promoter operably linked to a nucleotide sequence encoding an exogenous FOXP3 protein or a functional derivative thereof, and which constitutively expresses the FOXP3 gene product at a FOXP3 expression level that is equal to or greater than the FOXP3 expression level of a naturally occurring regulatory T (Treg) cell, wherein the inserted nucleic acid molecule encoding the constitutively active promoter or encoding the constitutively active promoter operably linked to the nucleotide sequence encoding exogenous FoxP3 protein or functional derivative thereof further comprises a nucleic acid sequence encoding a first chemically inducible signaling complex (CISC) component capable of specifically binding to a CISC inducer molecule; and (b) a native T-cell receptor alpha (TRAC) locus that has been knocked out and into which TRAC locus has been inserted, by homology-directed repair, at least one transduced polynucleotide encoding an exogenous antigen-specific T cell receptor (TCR) polypeptide, wherein the transduced nucleic acid sequence encoding the exogenous TCR polypeptide further comprises a nucleic acid sequence encoding a second chemically inducible signaling complex (CISC) component that is different from the first CISC component and is capable of specifically binding to the CISC inducer molecule. It is to be understood that methods described herein that comprise manipulation of CD4+ cells, can be applied to other types of cells (e.g., CD8+ cells). In some embodiments, the methods provided herein comprise editing CD3+ cells, thereby producing edited CD3+ cells, including CD4+ and CD8+ airT cells. In some embodiments, the methods comprise editing CD4+ T cells, thereby producing CD4+ airT cells. In some embodiments, the methods comprise editing CD8+ T cells, thereby producing CD8+ airT cells. In some embodiments, the methods comprise editing NK1.1+ T cells, thereby producing NK1.1+ airT cells. In some embodiments, the methods comprise editing CD34+ hematopoietic stem cells (HSCs). In some embodiments, the methods comprise editing induced pluripotent stem cells (iPSCs). Edited stem cells may be matured in vitro to produce airT cells, or administered to a subject to allow in vivo development into airT cells. Edited stem cells may be matured into CD3+ airT cells, CD4+ airT cells, CD8+ airT cells, NK1.1+ airT cells, or a combination thereof.

In some embodiments at least one of the nucleic acid sequences encoding the first CISC component and the nucleic acid sequence encoding the second CISC component further comprises a nucleic acid sequence encoding a third CISC component that is different from the first and second CISC components and is capable of specifically binding to the CISC inducer molecule. In some embodiments, the third CISC component is soluble and does not comprise a transmembrane domain or an extracellular domain. In some embodiments, the soluble third CISC component does not comprise a secretory peptide and is localized in the cytoplasm of the cell. In some embodiments, the nucleic acid sequence encoding the first CISC component comprises nucleic acid sequences with homology to a first locus in a genome of a cell, and the nucleic acid sequence encoding the second CISC component comprises nucleic acid sequences with homology to a second locus in the genome of the cell, with the first and second loci being different loci. In some embodiments, the first locus is a FOXP3 locus, a TRAC locus, an AAVS1 locus, or a ROSA26 locus; and the second locus is a FOXP3 locus, a TRAC locus, an AAVS1 locus, or a ROSA26 locus, where the second locus is different from the first locus. In some embodiments, the first and second loci are the same locus (e.g., upstream or downstream from each other in the same gene locus), wherein the first and second loci are selected from the group consisting of a FOXP3 locus, a TRAC locus, a an AAVS1 locus, and a ROSA26 locus. In some embodiments, nucleic acids comprising nucleotide sequences encoding the first or second CISC components, and optionally a third CISC component are provided. In some embodiments, one or more nucleic acids comprised in a vector are provided. In some embodiments, cells comprising one or more of the nucleic acids, vectors, or a genome modified by insertion of a vector or nucleic acid are provided herein. In some embodiments, compositions comprising nucleic acids, vectors, or genetically modified cells are provided.

In some embodiments, a first polynucleotide encoding a first CISC component is inserted in first locus (e.g., Foxp3 locus) and a second polynucleotide encoding a second CISC component is inserted in second locus (e.g., TRAC locus). In some embodiments, a first polynucleotide is comprised on a first nucleic acid vector and a second polynucleotide is comprised on a second nucleic acid vector. In some embodiments, a first polynucleotide and second polynucleotide are comprised on the same nucleic acid vector. In some embodiments, a first polynucleotide further comprises one or more regulatory elements (e.g., a heterologous promoter such as MND) and/or a payload (e.g., a nucleic acid encoding a FOXP3 polypeptide). In some embodiments, a second polynucleotide further comprises one or more regulatory elements (e.g., a heterologous promoter such as MND) and/or a payload (e.g., a nucleic acid encoding a TCR or CAR). In some embodiments, a first CISC component to be encoded from a first locus comprises an extracellular domain comprising FKBP or functional fragment thereof, and a second CISC component to be encoded from a first locus comprises an extracellular domain comprising FRB or functional fragment thereof). In some embodiments, a first CISC component to be encoded from a first locus comprises an extracellular domain comprising FRB or functional fragment thereof, and a second CISC component to be encoded from a first locus comprises an extracellular domain comprising FKBP or functional fragment thereof. In some embodiments, a CISC component with an extracellular domain comprising FKBP or a functional fragment thereof also comprises an IL-2RB intracellular signaling domain or a functional fragment thereof. In some embodiments, a CISC component with an extracellular domain comprising FKBP or a functional fragment thereof also comprises an IL-2RG intracellular signaling domain or a functional fragment thereof. In some embodiments, a CISC component with an extracellular domain comprising FRB or a functional fragment thereof also comprises an IL-2RB intracellular signaling domain or a functional fragment thereof. In some embodiments, a CISC component with an extracellular domain comprising FRB or a functional fragment thereof also comprises an IL-2RB intracellular signaling domain or a functional fragment thereof. In some embodiments, a first or second polynucleotide further comprises a nucleic acid encoding a third CISC component that comprises FRB or functional fragment thereof and does not comprise an extracellular or transmembrane domain. A functional fragment of FKBP or FRB is one that can bind to rapamycin or rapalog.

In some embodiments the airT cell comprises at least a first and a second transduced polynucleotide each encoding an antigen-specific TCR polypeptide, wherein said first transduced polynucleotide encodes a TCR V-alpha polypeptide and said second transduced polynucleotide encodes a TCR V-beta polypeptide, wherein said V-alpha polypeptide and said V-beta polypeptide comprise a functional TCR capable of specific antigen recognition. In some embodiments the airT cell expresses an antigen-specific T cell receptor (TCR) comprising the antigen-specific TCR polypeptide encoded by the at least one transduced polynucleotide encoding said TCR polypeptide and which is capable of antigen-specifically induced immunosuppression in response to HLA-restricted stimulation by an antigen that is specifically recognized by said TCR polypeptide. In some embodiments the antigen-specifically induced immunosuppression comprises one or more of: (i) inhibition of either or both of activation and proliferation of effector T cells that recognize the antigen that is specifically recognized by the airT TCR comprising the TCR polypeptide that is encoded by the at least one transduced polynucleotide, (ii) inhibition of expression of inflammatory cytokines or inflammatory mediators by effector T cells that recognize the antigen that is specifically recognized by the airT TCR comprising the TCR polypeptide that is encoded by the at least one transduced polynucleotide (iii) elaboration of one or more immunosuppressive cytokines, perforin/granzyme, or anti-inflammatory products by the airT cell or induction in the airT cell of at least one of indoleamine 2,3-dioxygenase (IDO), competition for IL2 or adenosine, catabolism of tryptophan, and expression of inhibitory receptors, and (iv) inhibition of either or both of activation and proliferation of effector T cells that do not recognize the antigen that is specifically recognized by the airT TCR comprising the TCR polypeptide that is encoded by the at least one transduced polynucleotide.

In some embodiments the TCR specifically recognizes an antigen associated with pathogenesis of an autoimmune condition, an allergic condition, or an inflammatory condition. In some embodiments the TCR specifically recognizes an antigen associated with pathogenesis of an autoimmune disease (e.g., diabetes such as type-1 diabetes, primary biliary cholangitis), autoinflammatory disease (e.g., ARDS, stroke, and atherosclerotic cardiovascular disease), alloimmune disease (e.g., graft-versus-host disease, sold organ transplant, and immune mediated recurrent pregnancy loss), and/or allergic disease (e.g., asthma, drug hypersensitivity, and celiac disease). In some embodiments, a condition to be treated is a cancer. Wang et al. (J Intern Med. 2015 October;278(4):369-95) provide a review of autoimmune diseases, which review is incorporated herein by reference. In some embodiments (i) the autoimmune condition is selected from type 1 diabetes mellitus, multiple sclerosis, systemic lupus erythematosus, myasthenia gravis, rheumatoid arthritis, early onset rheumatoid arthritis, ankylosing spondylitis, immune-mediated pregnancy loss, immune-mediated recurrent pregnancy loss, dermatomyositis, psoriatic arthritis, Crohn's disease, inflammatory bowel disease (IBD), ulcerative colitis, bullous pemphigoid, pemphigus vulgaris, autoimmune hepatitis, psoriasis, Sjogren's syndrome, or celiac disease; (ii) the allergic condition is selected from allergic asthma, atopic dermatitis, pollen allergy, food allergy, drug hypersensitivity, or contact dermatitis; and (iii) the inflammatory condition is selected from pancreatic islet cell transplantation, asthma, steroid-resistant asthma, hepatitis, traumatic brain injury, primary sclerosing cholangitis, primary biliary cholangitis, polymyositis, stroke, Still's disease, acute respiratory distress syndrome, uveitis, inflammatory bowel disease (IBD), ulcerative colitis, graft-versus-host disease (GVHD), tolerance induction for transplantation, transplant rejection, or sepsis. In some embodiments, the inflammatory condition is primary biliary cholangitis. In some embodiments, the inflammatory condition is primary sclerosing cholangitis. In some embodiments, the inflammatory condition is autoimmune hepatitis. In some embodiments, the autoimmune condition is type 1 diabetes. In some embodiments, the inflammatory condition is islet cell transplantation. In some embodiments, the inflammatory condition is transplant rejection. In some embodiments, the autoimmune condition is multiple sclerosis. In some embodiments, the inflammatory condition is inflammatory bowel disease. In some embodiments, the inflammatory condition is acute respiratory distress syndrome. In some embodiments, the inflammatory condition is stroke. In some embodiments, the inflammatory condition is graft-versus-host disease. In some embodiments (i) the antigen associated with pathogenesis of the autoimmune condition is selected from an autoantigen set forth in any one or more of FIGS. 141-144, (ii) the antigen associated with pathogenesis of the allergic condition is selected from an allergenic antigen set forth in any one or more of FIGS. 141-144, and (iii) the antigen associated with pathogenesis of the inflammatory condition is selected from an inflammation-associated antigen set forth in any one or more of FIGS. 141-144.

In some embodiments the airT cell comprises at least one transduced polynucleotide sequence encoding a TCR polypeptide that specifically binds in a human HLA-restricted manner to an antigenic polypeptide epitope of no more than 35, 34, 33, 32, 31, 30, 29, 28, 27, 26, 25, 24, 23, 22, 21, 20, 19, 18, 17, 16, 15, 14, 13, 12, 11, 10, 9, 8, or 7 consecutive amino acids of an amino acid sequence selected from any one of the antigenic polypeptide sequences set forth in any one or more of FIGS. 141-144, or that is encoded by a nucleotide sequence set forth in any one or more of FIG. 139A or 140A. In some embodiments the airT cell comprises at least a first and a second transduced polynucleotide sequence encoding, respectively, a TCR V-alpha polypeptide and a TCR V-beta polypeptide of a TCR that specifically binds in a human HLA-restricted manner to an antigenic polypeptide epitope of no more than 35, 34, 33, 32, 31, 30, 29, 28, 27, 26, 25, 24, 23, 22, 21, 20, 19, 18, 17, 16, 15, 14, 13, 12, 11, 10, 9, 8, or 7 consecutive amino acids of an amino acid sequence selected from any one of the antigenic polypeptide sequences set forth in any one or more of FIGS. 141-144, or that comprises any one TCR-alpha polypeptide sequence set forth in any one or more of FIGS. 136-140 or encoded by a nucleotide sequence set forth in any one or more of FIGS. 139-140. In some embodiments the airT cell comprises at least a first and a second transduced polynucleotide sequence encoding, respectively, a TCR V-alpha polypeptide and a TCR V-beta polypeptide of a TCR that specifically binds in a human HLA-restricted manner to an antigenic polypeptide, wherein the TCR V-alpha and V-beta polypeptides comprise paired sequences selected from any one paired TCR V-alpha and V-beta polypeptide sequences set forth in FIG. 143.

In some embodiments the cell exhibits an induced level of Treg biological activity that is increased in response to MHC-restricted stimulation of the airT cell by an antigen recognized by the TCR polypeptide encoded by the at least one transduced polynucleotide, relative to a control level of Treg biological activity that is exhibited by the airT cell without MHC-restricted stimulation by the antigen, wherein the Treg biological activity comprises one or more of: (i) inhibition of either or both of activation and proliferation of effector T cells that recognize the antigen that is specifically recognized by the airT TCR comprising the TCR polypeptide that is encoded by the at least one transduced polynucleotide, (ii) inhibition of expression of inflammatory cytokines or inflammatory mediators by effector T cells that recognize the antigen that is specifically recognized by the airT TCR comprising the TCR polypeptide that is encoded by the at least one transduced polynucleotide (iii) elaboration of one or more immunosuppressive cytokines, perforin/granzyme, or anti-inflammatory products by the airT cell or induction in the airT cell of at least one of indoleamine 2,3-dioxygenase (IDO), competition for IL2 or adenosine, catabolism of tryptophan, expression of inhibitory receptors, or (iv) inhibition of either or both of activation and proliferation of effector T cells that do not recognize the antigen that is specifically recognized by the airT TCR comprising the TCR polypeptide that is encoded by the at least one transduced polynucleotide.

In some embodiments, (1) the antigen associated with pathogenesis of an autoimmune condition is IGRP(241-270) peptide, wherein the autoimmune condition is type 1 diabetes, and wherein the TCR is TCR T1D4 recognizing the IGRP(241-270) peptide in an HLA DRB1*0404-restricted manner; (2) the antigen associated with pathogenesis of an autoimmune condition is IGRP(305-324) peptide, wherein the autoimmune condition is type 1 diabetes, and wherein the TCR is TCR T1D5 recognizing the IGRP(305-324) peptide in an HLA DRB1*0404-restricted manner; or (3) the antigen associated with pathogenesis of an autoimmune condition is IGRP(305-324) peptide, wherein the autoimmune condition is type 1 diabetes, and wherein the TCR is TCR T1D2 recognizing the IGRP(305-324) peptide in an HLA DRB1*0404-restricted manner.

Some embodiments of the methods and compositions provided herein include any one of the foregoing airT cells or airT cells disclosed anywhere herein for use in the treatment, inhibition, or amelioration of an autoimmune, allergic, or inflammatory condition, such as one selected from type 1 diabetes mellitus, multiple sclerosis, systemic lupus erythematosus, myasthenia gravis, rheumatoid arthritis, early onset rheumatoid arthritis, ankylosing spondylitis, immune-mediated pregnancy loss, immune-mediated recurrent pregnancy loss, dermatomyositis, psoriatic arthritis, Crohn's disease, inflammatory bowel disease (IBD), ulcerative colitis, bullous pemphigoid, pemphigus vulgaris, autoimmune hepatitis, psoriasis, Sjogren's syndrome, celiac disease, allergic asthma, atopic dermatitis, pollen allergy, food allergy, drug hypersensitivity, contact dermatitis, pancreatic islet cell transplantation, asthma, steroid-resistant asthma, hepatitis, traumatic brain injury, primary sclerosing cholangitis, primary biliary cholangitis, polymyositis, stroke, Still's disease, acute respiratory distress syndrome, uveitis, inflammatory bowel disease (IBD), ulcerative colitis, graft-versus-host disease (GvHD), tolerance induction for transplantation, transplant rejection, or sepsis. Some embodiments of the methods and compositions provided herein include any one of the foregoing airT cells or airT cells disclosed anywhere herein for use in the treatment, inhibition, or amelioration of an autoimmune disease (e.g., diabetes such as type-1 diabetes, primary biliary cholangitis), autoinflammatory disease (e.g., ARDS, stroke, and atherosclerotic cardiovascular disease), alloimmune disease (e.g., graft-versus-host disease, sold organ transplant, and immune mediated recurrent pregnancy loss), and/or allergic disease (e.g., asthma, drug hypersensitivity, and celiac disease). In some embodiments, the inflammatory condition is primary biliary cholangitis. In some embodiments, the inflammatory condition is primary sclerosing cholangitis. In some embodiments, the inflammatory condition is autoimmune hepatitis. In some embodiments, the autoimmune condition is type 1 diabetes. In some embodiments, the inflammatory condition is islet cell transplantation. In some embodiments, the inflammatory condition is transplant rejection. In some embodiments, the autoimmune condition is multiple sclerosis. In some embodiments, the inflammatory condition is inflammatory bowel disease. In some embodiments, the inflammatory condition is acute respiratory distress syndrome. In some embodiments, the inflammatory condition is stroke. In some embodiments, the inflammatory condition is graft-versus-host disease. In some embodiments, a condition to be treated is a cancer. Wang et al. (J Intern Med. 2015 October;278(4):369-95) provide a review of autoimmune diseases, which review is incorporated herein by reference. In some embodiments, the TCR polypeptide binds to an antigen associated with a disorder selected from type 1 diabetes mellitus, multiple sclerosis, myocarditis, rheumatoid arthritis (RA), or systemic lupus erythematosus (SLE).

In some embodiments, the antigen is selected from the group consisting of vimentin, aggrecan, cartilage intermediate layer protein (CILP), preproinsulin, islet-specific glucose-6-phosphatase catalytic subunit-related protein (IGRP), and enolase.

In some embodiments, the antigen comprises an epitope selected from the group consisting of Enol326, CILP297-1, Vim418, Agg520, and SLE3.

In some embodiments, the antigen comprises an epitope having the amino acid sequence of any one of SEQ ID NOs 1363-1376 and 1408-1415.

In some embodiments, the TCR polypeptide comprises: a CD3 alpha polypeptide having the amino acid sequence of any one of SEQ ID NOs 1377-1390; and/or a CD3 beta polypeptide having the amino acid sequence of any one of SEQ ID NOs 1377-1390.

Some embodiments of the methods and compositions provided herein include a pharmaceutical composition comprising any one of the foregoing airT cells and a pharmaceutically acceptable excipient.

Some embodiments of the methods and compositions provided herein include use of any one of the foregoing airT cells as a medicament.

In some embodiments there is provided a method of producing an artificial antigen-specific immunoregulatory T (airT) cell, comprising: (a) introducing into a CD4+ T cell (1) a FOXP3 guide RNA (gRNA) comprising a spacer sequence complementary to a sequence within a native forkhead box protein 3/winged helix transcription factor (FOXP3) gene in the cell, or a nucleic acid encoding the FOXP3 gRNA; (2) a DNA endonuclease capable of forming a complex with the FOXP3 gRNA of (1), or a nucleic acid encoding the DNA endonuclease; and (3) a FOXP3 locus donor template selected from (i) a nucleic acid molecule comprising a constitutively active promoter capable of promoting transcription of an endogenous FOXP3-encoding nucleotide sequence of the FOXP3 gene; and (ii) a nucleic acid molecule comprising a constitutively active promoter operably linked to a nucleotide sequence encoding a FOXP3 protein or a functional derivative thereof, under conditions and for a time sufficient for knock-out or inactivation of the native FOXP3 gene locus in the cell and insertion of all or a portion of the FOXP3 locus donor template nucleic acid (e.g., by HDR or NHEJ); and (b) simultaneously or sequentially and in any order with (a), transducing the CD4+ T cell with at least one polynucleotide encoding an antigen-specific T cell receptor (TCR) polypeptide. In some embodiments step (b) is selected from: (i) transducing the CD4+ T cell with at least one vector comprising the polynucleotide encoding the antigen-specific T cell receptor (TCR) or chimeric antigen receptor (CAR) polypeptide, and (ii) introducing into the CD4+ T cell (1) a T cell receptor alpha (TRAC) guide RNA (gRNA) comprising a spacer sequence complementary to a sequence within a native TRAC gene locus in the cell, or a nucleic acid encoding the TRAC gRNA; (2) a DNA endonuclease capable of forming a complex with the TRAC gRNA of (1), or a nucleic acid encoding the DNA endonuclease; and (3) a TRAC locus donor template comprising the at least one polynucleotide encoding the antigen-specific T cell receptor (TCR) or chimeric antigen receptor (CAR) polypeptide, under conditions and for a time sufficient for knock-out or inactivation of the native TRAC gene locus in the cell and insertion of all or a portion of the TRAC locus donor template nucleic acid (e.g., by HDR or NHEJ). In some embodiments, the FOXP3 locus donor template comprises a polynucleotide/nucleic acid encoding a first CISC component, and the TRAC locus donor template comprises a polynucleotide/nucleic acid encoding a second CISC component that is complementary to the first CISC component, wherein each of the first and second CISC components are capable of specifically binding to a CISC inducer molecule, and wherein the first and second CISC components dimerize in the presence of the CISC inducer molecule. In other embodiments, the FOXP3 locus donor template encodes a second CISC component, and the TRAC locus donor template encodes a first CISC component, where both CISC components are capable of specifically binding to a CISC inducer molecule such that they dimerize in the presence of the CISC inducer molecule. The first and second CISC components encoded by the respective donor templates may be any of the first and second CISC components provided herein. In some embodiments, the FOXP3 locus donor template and/or the TRAC locus donor template comprises a polynucleotide/nucleic acid encoding a third CISC component that is soluble, cytosolic, and capable of specifically binding to the CISC inducer molecule. Any method for gene editing is contemplated herein, including, without limitation, viral and non-viral approaches that enable targeted gene-editing (e.g., Cas9, ZFN, TALEN). Accordingly, a method of gene editing as provided herein may make use of a nuclease to target a locus or a targeted locus on a nucleic acid sequence. In some embodiments, a nuclease is an RNA-guided nuclease (e.g., a CRISPR/Cas nuclease), a meganuclease, a zinc-finger nuclease, or TALEN. [0057] In some embodiments there is provided a method of producing an artificial antigen-specific immunoregulatory T (airT) cell, comprising: (a) introducing into a CD4+ T cell (1) a first T cell receptor alpha (TRAC) guide RNA (gRNA) comprising a first spacer sequence complementary to a first sequence within a native TRAC gene locus in the cell, or a nucleic acid encoding the first TRAC gRNA; (2) a first DNA endonuclease capable of forming a complex with the first TRAC gRNA of (1), or a nucleic acid encoding the first DNA endonuclease; and (3) a first TRAC locus donor template selected from (i) a nucleic acid molecule comprising a nucleotide sequence encoding a FOXP3 protein or a functional derivative thereof, and (ii) a nucleic acid molecule comprising a constitutively active promoter operably linked to a nucleotide sequence encoding a FOXP3 protein or a functional derivative thereof, under conditions and for a time sufficient for knock-out of the native TRAC gene locus in the cell and insertion of all or a portion of the first TRAC locus donor template nucleic acid (e.g., by HDR or NHEJ); and (b) simultaneously or sequentially and in any order with (a), introducing into the CD4+ T cell (1) a second T cell receptor alpha (TRAC) guide RNA (gRNA) comprising a second spacer sequence complementary to a second sequence within a TRAC gene, or a nucleic acid encoding the second TRAC gRNA, wherein the second spacer sequence is not identical to the first spacer sequence; (2) a second DNA endonuclease capable of forming a complex with the second TRAC gRNA of (1), or a nucleic acid encoding the second DNA endonuclease, wherein the second DNA endonuclease is selected from a DNA endonuclease that is identical to the first DNA endonuclease and a DNA endonuclease that is not identical to the first DNA endonuclease; and (3) a second TRAC locus donor template comprising the at least one polynucleotide encoding the antigen-specific T cell receptor (TCR) polypeptide, under conditions and for a time sufficient for knock-out or inactivation of the native TRAC gene locus in the cell and insertion of all or a portion of the second TRAC locus donor template nucleic acid (e.g., by HDR or NHEJ). In some embodiments, the FOXP3 locus donor template comprises a polynucleotide/nucleic acid encoding a first CISC component, and the TRAC locus donor template comprises a polynucleotide/nucleic acid encoding a second CISC component that is complementary to the first CISC component, wherein each of the first and second CISC components are capable of specifically binding to a CISC inducer molecule, and wherein the first and second CISC components dimerize in the presence of the CISC inducer molecule. In other embodiments, the FOXP3 locus donor template encodes a second CISC component, and the TRAC locus donor template encodes a first CISC component, where both CISC components are capable of specifically binding to a CISC inducer molecule such that they dimerize in the presence of the CISC inducer molecule. The first and second CISC components encoded by the respective donor templates may be any of the first and second CISC components provided herein. In some embodiments, the FOXP3 locus donor template and/or the TRAC locus donor template comprises a polynucleotide encoding a third CISC component that is soluble, cytosolic, and capable of specifically binding to the CISC inducer molecule. Any method for gene editing is contemplated herein, including, without limitation, viral and non-viral approaches that enable targeted gene-editing (e.g., Cas9, ZFN, TALEN). Accordingly, a method of gene editing as provided herein may make use of a nuclease to target a locus or a targeted locus on a nucleic acid sequence. In some embodiments, a nuclease is an RNA-guided nuclease (e.g., a CRISPR/Cas nuclease), a meganuclease, a zinc-finger nuclease, or TALEN.

In some embodiments there is provided a method of producing an artificial antigen-specific immunoregulatory T (airT) cell, comprising: introducing into a CD4+ T cell (1) a T cell receptor alpha (TRAC) guide RNA (gRNA) comprising a spacer sequence that is complementary to a sequence within a native TRAC gene locus in the cell, or a nucleic acid encoding the TRAC gRNA; (2) a DNA endonuclease that is capable of forming a complex with the TRAC gRNA of (1), or a nucleic acid encoding the DNA endonuclease; and (3) a TRAC locus donor template which comprises the at least one polynucleotide that encodes the antigen-specific T cell receptor (TCR) polypeptide, under conditions and for a time sufficient for knock-out or inactivation of the native TRAC gene locus in the cell and insertion of all or a portion of the TRAC locus donor template by homology-directed repair (HDR) or non-homologous end-joining (NHEJ). In some embodiments a first one of said insertion donor templates further comprises a nucleic acid sequence encoding a first chemically inducible signaling complex (CISC) component that is capable of specifically binding to a CISC inducer molecule, and a second one of said insertion donor templates further comprises a nucleic acid sequence encoding a second chemically inducible signaling complex (CISC) component that is different from the first CISC component and is capable of specifically binding to the CISC inducer molecule. In some embodiments the first insertion donor template comprises homology to a first locus (e.g., the Foxp3 or TRAC locus) in a genome of a cell, and the second insertion donor template comprises homology to a second locus (e.g., the Foxp3 or TRAC locus) in a genome of a cell, and optionally the first and second loci are different loci. The first and second loci can be any loci in a genome, e.g., other than Foxp3 or TRAC locus. In some embodiments at least one of the first and second insertion donor templates further comprises a nucleic acid sequence encoding a third CISC component that is different from the first and second CISC components and is capable of specifically binding to the CISC inducer molecule. In some embodiments of the methods provided herein: (a) the DNA endonuclease is selected from a an RNA-guided nuclease (RGN), CRISPR/Cas nuclease, a TALEN, a meganuclease, megaTAL, or a zinc finger nuclease, (b) the constitutively active promoter is MND, insertion is by a mechanism selected from homology-directed repair or non-homologous end joining, (d) the first and second CISC components comprise intracellular domains that are selected in a mutually exclusive manner from IL2RB or IL2RG, and comprise extracellular domains that are selected in a mutually exclusive manner from FKBP and FRB, (e) the third CISC component is FRB, which is encoded by either the first or second donor template, (f) the CISC inducer molecule is rapamycin or an analog thereof, and/or (g) the first and second donor template are inserted into two distinct loci, wherein each locus is selected independently from the group consisting of a FOXP3 locus, a TRAC locus, an AAVS1 locus, and a ROSA26 locus. Any method for gene editing is contemplated herein, including, without limitation, viral and non-viral approaches that enable targeted gene-editing (e.g., Cas9, ZFN, TALEN). Accordingly, a method of gene editing as provided herein may make use of a nuclease to target a locus or a targeted locus on a nucleic acid sequence. In some embodiments, a nuclease is Cas9, a zinc-finger nuclease or TALEN.

In some embodiments there is provided a method of producing an artificial antigen-specific immunoregulatory T (airT) cell, comprising: (a) introducing into a CD8+ T cell (1) a FOXP3 guide RNA (gRNA) comprising a spacer sequence complementary to a sequence within a native forkhead box protein 3/winged helix transcription factor (FOXP3) gene in the cell, or a nucleic acid encoding the FOXP3 gRNA; (2) a DNA endonuclease capable of forming a complex with the FOXP3 gRNA of (1), or a nucleic acid encoding the DNA endonuclease; and (3) a FOXP3 locus donor template selected from (i) a nucleic acid molecule comprising a constitutively active promoter capable of promoting transcription of an endogenous FOXP3-encoding nucleotide sequence of the FOXP3 gene; and (ii) a nucleic acid molecule comprising a constitutively active promoter operably linked to a nucleotide sequence encoding a FOXP3 protein or a functional derivative thereof, under conditions and for a time sufficient for knock-out or inactivation of the native FOXP3 gene locus in the cell and insertion of all or a portion of the FOXP3 locus donor template nucleic acid (e.g., by HDR or NHEJ); and (b) simultaneously or sequentially and in any order with (a), transducing the CD8+ T cell with at least one polynucleotide encoding an antigen-specific T cell receptor (TCR) polypeptide. In some embodiments step (b) is selected from: (i) transducing the CD8+ T cell with at least one vector comprising the polynucleotide encoding the antigen-specific T cell receptor (TCR) or chimeric antigen receptor (CAR) polypeptide, and (ii) introducing into the CD8+ T cell (1) a T cell receptor alpha (TRAC) guide RNA (gRNA) comprising a spacer sequence complementary to a sequence within a native TRAC gene locus in the cell, or a nucleic acid encoding the TRAC gRNA; (2) a DNA endonuclease capable of forming a complex with the TRAC gRNA of (1), or a nucleic acid encoding the DNA endonuclease; and (3) a TRAC locus donor template comprising the at least one polynucleotide/nucleic acid encoding the antigen-specific T cell receptor (TCR) or chimeric antigen receptor (CAR) polypeptide, under conditions and for a time sufficient for knock-out or inactivation of the native TRAC gene locus in the cell and insertion of all or a portion of the TRAC locus donor template nucleic acid (e.g., by HDR or NHEJ). In some embodiments, the FOXP3 locus donor template comprises a polynucleotide/nucleic acid encoding a first CISC component, and the TRAC locus donor template comprises a polynucleotide/nucleic acid encoding a second CISC component that is complementary to the first CISC component, wherein each of the first and second CISC components are capable of specifically binding to a CISC inducer molecule, and wherein the first and second CISC components dimerize in the presence of the CISC inducer molecule. In other embodiments, the FOXP3 locus donor template encodes a second CISC component, and the TRAC locus donor template encodes a first CISC component, where both CISC components are capable of specifically binding to a CISC inducer molecule such that they dimerize in the presence of the CISC inducer molecule. The first and second CISC components encoded by the respective donor templates may be any of the first and second CISC components provided herein. In some embodiments, the FOXP3 locus donor template and/or the TRAC locus donor template comprises a polynucleotide/nucleic acid encoding a third CISC component that is soluble, cytosolic, and capable of specifically binding to the CISC inducer molecule. Any method for gene editing is contemplated herein, including, without limitation, viral and non-viral approaches that enable targeted gene-editing (e.g., Cas9, ZFN, TALEN). Accordingly, a method of gene editing as provided herein may make use of a nuclease to target a locus or a targeted locus on a nucleic acid sequence. In some embodiments, a nuclease is an RNA-guided nuclease (e.g., a CRISPR/Cas nuclease), a meganuclease, a zinc-finger nuclease, or TALEN. [0057] In some embodiments there is provided a method of producing an artificial antigen-specific immunoregulatory T (airT) cell, comprising: (a) introducing into a CD8+ T cell (1) a first T cell receptor alpha (TRAC) guide RNA (gRNA) comprising a first spacer sequence complementary to a first sequence within a native TRAC gene locus in the cell, or a nucleic acid encoding the first TRAC gRNA; (2) a first DNA endonuclease capable of forming a complex with the first TRAC gRNA of (1), or a nucleic acid encoding the first DNA endonuclease; and (3) a first TRAC locus donor template selected from (i) a nucleic acid molecule comprising a nucleotide sequence encoding a FOXP3 protein or a functional derivative thereof, and (ii) a nucleic acid molecule comprising a constitutively active promoter operably linked to a nucleotide sequence encoding a FOXP3 protein or a functional derivative thereof, under conditions and for a time sufficient for knock-out of the native TRAC gene locus in the cell and insertion of all or a portion of the first TRAC locus donor template nucleic acid (e.g., by HDR or NHEJ); and (b) simultaneously or sequentially and in any order with (a), introducing into the CD8+ T cell (1) a second T cell receptor alpha (TRAC) guide RNA (gRNA) comprising a second spacer sequence complementary to a second sequence within a TRAC gene, or a nucleic acid encoding the second TRAC gRNA, wherein the second spacer sequence is not identical to the first spacer sequence; (2) a second DNA endonuclease capable of forming a complex with the second TRAC gRNA of (1), or a nucleic acid encoding the second DNA endonuclease, wherein the second DNA endonuclease is selected from a DNA endonuclease that is identical to the first DNA endonuclease and a DNA endonuclease that is not identical to the first DNA endonuclease; and (3) a second TRAC locus donor template comprising the at least one polynucleotide/nucleic acid encoding the antigen-specific T cell receptor (TCR) polypeptide, under conditions and for a time sufficient for knock-out or inactivation of the native TRAC gene locus in the cell and insertion of all or a portion of the second TRAC locus donor template nucleic acid (e.g., by HDR or NHEJ). In some embodiments, the FOXP3 locus donor template comprises a polynucleotide/nucleic acid encoding a first CISC component, and the TRAC locus donor template comprises a polynucleotide/nucleic acid encoding a second CISC component that is complementary to the first CISC component, wherein each of the first and second CISC components are capable of specifically binding to a CISC inducer molecule, and wherein the first and second CISC components dimerize in the presence of the CISC inducer molecule. In other embodiments, the FOXP3 locus donor template encodes a second CISC component, and the TRAC locus donor template encodes a first CISC component, where both CISC components are capable of specifically binding to a CISC inducer molecule such that they dimerize in the presence of the CISC inducer molecule. The first and second CISC components encoded by the respective donor templates may be any of the first and second CISC components provided herein. In some embodiments, the FOXP3 locus donor template and/or the TRAC locus donor template comprises a polynucleotide/nucleic acid encoding a third CISC component that is soluble, cytosolic, and capable of specifically binding to the CISC inducer molecule. Any method for gene editing is contemplated herein, including, without limitation, viral and non-viral approaches that enable targeted gene-editing (e.g., Cas9, ZFN, TALEN). Accordingly, a method of gene editing as provided herein may make use of a nuclease to target a locus or a targeted locus on a nucleic acid sequence. In some embodiments, a nuclease is an RNA-guided nuclease (e.g., a CRISPR/Cas nuclease), a meganuclease, a zinc-finger nuclease, or TALEN.

In some embodiments there is provided a method of producing an artificial antigen-specific immunoregulatory T (airT) cell, comprising: introducing into a CD8+ T cell (1) a T cell receptor alpha (TRAC) guide RNA (gRNA) comprising a spacer sequence that is complementary to a sequence within a native TRAC gene locus in the cell, or a nucleic acid encoding the TRAC gRNA; (2) a DNA endonuclease that is capable of forming a complex with the TRAC gRNA of (1), or a nucleic acid encoding the DNA endonuclease; and (3) a TRAC locus donor template which comprises the at least one polynucleotide/nucleic acid that encodes the antigen-specific T cell receptor (TCR) polypeptide, under conditions and for a time sufficient for knock-out or inactivation of the native TRAC gene locus in the cell and insertion of all or a portion of the TRAC locus donor template by homology-directed repair (HDR) or non-homologous end-joining (NHEJ). In some embodiments a first one of said insertion donor templates further comprises a nucleic acid sequence encoding a first chemically inducible signaling complex (CISC) component that is capable of specifically binding to a CISC inducer molecule, and a second one of said insertion donor templates further comprises a nucleic acid sequence encoding a second chemically inducible signaling complex (CISC) component that is different from the first CISC component and is capable of specifically binding to the CISC inducer molecule. In some embodiments the first insertion donor template comprises homology to a first locus (e.g., the Foxp3 or TRAC locus) in a genome of a cell, and the second insertion donor template comprises homology to a second locus (e.g., the Foxp3 or TRAC locus) in a genome of a cell, and optionally the first and second loci are different loci. In some embodiments at least one of the first and second insertion donor templates further comprises a nucleic acid sequence encoding a third CISC component that is different from the first and second CISC components and is capable of specifically binding to the CISC inducer molecule. In some embodiments of the methods provided herein: (a) the DNA endonuclease is selected from a an RNA-guided nuclease (RGN), CRISPR/Cas nuclease, a TALEN, a meganuclease, megaTAL, or a zinc finger nuclease, (b) the constitutively active promoter is MND, insertion is by a mechanism selected from homology-directed repair or non-homologous end joining, (d) the first and second CISC components comprise intracellular domains that are selected in a mutually exclusive manner from IL2RB or IL2RG, and comprise extracellular domains that are selected in a mutually exclusive manner from FKBP and FRB, (e) the third CISC component is FRB, which is encoded by either the first or second donor template, (f) the CISC inducer molecule is rapamycin or an analog thereof, and/or (g) the first and second donor template are inserted into two distinct loci, wherein each locus is selected independently from the group consisting of a FOXP3 locus, a TRAC locus, an AAVS1 locus, and a ROSA26 locus. Any method for gene editing is contemplated herein, including, without limitation, viral and non-viral approaches that enable targeted gene-editing (e.g., Cas9, ZFN, TALEN). Accordingly, a method of gene editing as provided herein may make use of a nuclease to target a locus or a targeted locus on a nucleic acid sequence. In some embodiments, a nuclease is Cas9, a zinc-finger nuclease or TALEN.

In some embodiments there is provided a method of producing an artificial antigen-specific immunoregulatory T (airT) cell, comprising: (a) introducing into a CD3+ T cell (1) a FOXP3 guide RNA (gRNA) comprising a spacer sequence complementary to a sequence within a native forkhead box protein 3/winged helix transcription factor (FOXP3) gene in the cell, or a nucleic acid encoding the FOXP3 gRNA; (2) a DNA endonuclease capable of forming a complex with the FOXP3 gRNA of (1), or a nucleic acid encoding the DNA endonuclease; and (3) a FOXP3 locus donor template selected from (i) a nucleic acid molecule comprising a constitutively active promoter capable of promoting transcription of an endogenous FOXP3-encoding nucleotide sequence of the FOXP3 gene; and (ii) a nucleic acid molecule comprising a constitutively active promoter operably linked to a nucleotide sequence encoding a FOXP3 protein or a functional derivative thereof, under conditions and for a time sufficient for knock-out or inactivation of the native FOXP3 gene locus in the cell and insertion of all or a portion of the FOXP3 locus donor template nucleic acid (e.g., by HDR or NHEJ); and (b) simultaneously or sequentially and in any order with (a), transducing the CD3+ T cell with at least one polynucleotide/nucleic acid encoding an antigen-specific T cell receptor (TCR) polypeptide. In some embodiments step (b) is selected from: (i) transducing the CD3+ T cell with at least one vector comprising the polynucleotide/nucleic acid encoding the antigen-specific T cell receptor (TCR) or chimeric antigen receptor (CAR) polypeptide, and (ii) introducing into the CD3+ T cell (1) a T cell receptor alpha (TRAC) guide RNA (gRNA) comprising a spacer sequence complementary to a sequence within a native TRAC gene locus in the cell, or a nucleic acid encoding the TRAC gRNA; (2) a DNA endonuclease capable of forming a complex with the TRAC gRNA of (1), or a nucleic acid encoding the DNA endonuclease; and (3) a TRAC locus donor template comprising the at least one polynucleotide/nucleic acid encoding the antigen-specific T cell receptor (TCR) or chimeric antigen receptor (CAR) polypeptide, under conditions and for a time sufficient for knock-out or inactivation of the native TRAC gene locus in the cell and insertion of all or a portion of the TRAC locus donor template nucleic acid (e.g., by HDR or NHEJ). In some embodiments, the FOXP3 locus donor template comprises a polynucleotide/nucleic acid encoding a first CISC component, and the TRAC locus donor template comprises a polynucleotide/nucleic acid encoding a second CISC component that is complementary to the first CISC component, wherein each of the first and second CISC components are capable of specifically binding to a CISC inducer molecule, and wherein the first and second CISC components dimerize in the presence of the CISC inducer molecule. In other embodiments, the FOXP3 locus donor template encodes a second CISC component, and the TRAC locus donor template encodes a first CISC component, where both CISC components are capable of specifically binding to a CISC inducer molecule such that they dimerize in the presence of the CISC inducer molecule. The first and second CISC components encoded by the respective donor templates may be any of the first and second CISC components provided herein. In some embodiments, the FOXP3 locus donor template and/or the TRAC locus donor template comprises a polynucleotide/nucleic acid encoding a third CISC component that is soluble, cytosolic, and capable of specifically binding to the CISC inducer molecule. Any method for gene editing is contemplated herein, including, without limitation, viral and non-viral approaches that enable targeted gene-editing (e.g., Cas9, ZFN, TALEN). Accordingly, a method of gene editing as provided herein may make use of a nuclease to target a locus or a targeted locus on a nucleic acid sequence. In some embodiments, a nuclease is an RNA-guided nuclease (e.g., a CRISPR/Cas nuclease), a meganuclease, a zinc-finger nuclease, or TALEN. [0057] In some embodiments there is provided a method of producing an artificial antigen-specific immunoregulatory T (airT) cell, comprising: (a) introducing into a CD3+ T cell (1) a first T cell receptor alpha (TRAC) guide RNA (gRNA) comprising a first spacer sequence complementary to a first sequence within a native TRAC gene locus in the cell, or a nucleic acid encoding the first TRAC gRNA; (2) a first DNA endonuclease capable of forming a complex with the first TRAC gRNA of (1), or a nucleic acid encoding the first DNA endonuclease; and (3) a first TRAC locus donor template selected from (i) a nucleic acid molecule comprising a nucleotide sequence encoding a FOXP3 protein or a functional derivative thereof, and (ii) a nucleic acid molecule comprising a constitutively active promoter operably linked to a nucleotide sequence encoding a FOXP3 protein or a functional derivative thereof, under conditions and for a time sufficient for knock-out of the native TRAC gene locus in the cell and insertion of all or a portion of the first TRAC locus donor template nucleic acid (e.g., by HDR or NHEJ); and (b) simultaneously or sequentially and in any order with (a), introducing into the CD3+ T cell (1) a second T cell receptor alpha (TRAC) guide RNA (gRNA) comprising a second spacer sequence complementary to a second sequence within a TRAC gene, or a nucleic acid encoding the second TRAC gRNA, wherein the second spacer sequence is not identical to the first spacer sequence; (2) a second DNA endonuclease capable of forming a complex with the second TRAC gRNA of (1), or a nucleic acid encoding the second DNA endonuclease, wherein the second DNA endonuclease is selected from a DNA endonuclease that is identical to the first DNA endonuclease and a DNA endonuclease that is not identical to the first DNA endonuclease; and (3) a second TRAC locus donor template comprising the at least one polynucleotide/nucleic acid encoding the antigen-specific T cell receptor (TCR) polypeptide, under conditions and for a time sufficient for knock-out or inactivation of the native TRAC gene locus in the cell and insertion of all or a portion of the second TRAC locus donor template nucleic acid (e.g., by HDR or NHEJ). In some embodiments, the FOXP3 locus donor template comprises a polynucleotide/nucleic acid encoding a first CISC component, and the TRAC locus donor template comprises a polynucleotide/nucleic acid encoding a second CISC component that is complementary to the first CISC component, wherein each of the first and second CISC components are capable of specifically binding to a CISC inducer molecule, and wherein the first and second CISC components dimerize in the presence of the CISC inducer molecule. In other embodiments, the FOXP3 locus donor template encodes a second CISC component, and the TRAC locus donor template encodes a first CISC component, where both CISC components are capable of specifically binding to a CISC inducer molecule such that they dimerize in the presence of the CISC inducer molecule. The first and second CISC components encoded by the respective donor templates may be any of the first and second CISC components provided herein. In some embodiments, the FOXP3 locus donor template and/or the TRAC locus donor template comprises a polynucleotide/nucleic acid encoding a third CISC component that is soluble, cytosolic, and capable of specifically binding to the CISC inducer molecule. Any method for gene editing is contemplated herein, including, without limitation, viral and non-viral approaches that enable targeted gene-editing (e.g., Cas9, ZFN, TALEN). Accordingly, a method of gene editing as provided herein may make use of a nuclease to target a locus or a targeted locus on a nucleic acid sequence. In some embodiments, a nuclease is an RNA-guided nuclease (e.g., a CRISPR/Cas nuclease), a meganuclease, a zinc-finger nuclease, or TALEN.

In some embodiments there is provided a method of producing an artificial antigen-specific immunoregulatory T (airT) cell, comprising: introducing into a CD3+ T cell (1) a T cell receptor alpha (TRAC) guide RNA (gRNA) comprising a spacer sequence that is complementary to a sequence within a native TRAC gene locus in the cell, or a nucleic acid encoding the TRAC gRNA; (2) a DNA endonuclease that is capable of forming a complex with the TRAC gRNA of (1), or a nucleic acid encoding the DNA endonuclease; and (3) a TRAC locus donor template which comprises the at least one polynucleotide/nucleic acid that encodes the antigen-specific T cell receptor (TCR) polypeptide, under conditions and for a time sufficient for knock-out or inactivation of the native TRAC gene locus in the cell and insertion of all or a portion of the TRAC locus donor template by homology-directed repair (HDR) or non-homologous end-joining (NHEJ). In some embodiments a first one of said insertion donor templates further comprises a nucleic acid sequence encoding a first chemically inducible signaling complex (CISC) component that is capable of specifically binding to a CISC inducer molecule, and a second one of said insertion donor templates further comprises a nucleic acid sequence encoding a second chemically inducible signaling complex (CISC) component that is different from the first CISC component and is capable of specifically binding to the CISC inducer molecule. In some embodiments the first insertion donor template comprises homology to a first locus (e.g., the Foxp3 or TRAC locus) in a genome of a cell, and the second insertion donor template comprises homology to a second locus (e.g., the Foxp3 or TRAC locus) in a genome of a cell, and optionally the first and second loci are different loci. In some embodiments at least one of the first and second insertion donor templates further comprises a nucleic acid sequence encoding a third CISC component that is different from the first and second CISC components and is capable of specifically binding to the CISC inducer molecule. In some embodiments of the methods provided herein: (a) the DNA endonuclease is selected from a an RNA-guided nuclease (RGN), CRISPR/Cas nuclease, a TALEN, a meganuclease, megaTAL, or a zinc finger nuclease, (b) the constitutively active promoter is MND, insertion is by a mechanism selected from homology-directed repair or non-homologous end joining, (d) the first and second CISC components comprise intracellular domains that are selected in a mutually exclusive manner from IL2RB or IL2RG, and comprise extracellular domains that are selected in a mutually exclusive manner from FKBP and FRB, (e) the third CISC component is FRB, which is encoded by either the first or second donor template, (f) the CISC inducer molecule is rapamycin or an analog thereof, and/or (g) the first and second donor template are inserted into two distinct loci, wherein each locus is selected independently from the group consisting of a FOXP3 locus, a TRAC locus, an AAVS1 locus, and a ROSA26 locus. Any method for gene editing is contemplated herein, including, without limitation, viral and non-viral approaches that enable targeted gene-editing (e.g., Cas9, ZFN, TALEN). Accordingly, a method of gene editing as provided herein may make use of a nuclease to target a locus or a targeted locus on a nucleic acid sequence. In some embodiments, a nuclease is Cas9, a zinc-finger nuclease or TALEN.

Some embodiments of the methods and compositions provided herein include a method of producing any one of the foregoing artificial antigen-specific immunoregulatory T (airT) cells, comprising performing any one of the foregoing methods of producing an artificial antigen-specific immunoregulatory T (airT) cell.

In some embodiments there is provided a method for treating, inhibiting, or ameliorating a subject having a condition in need of antigen-specific immunosuppression, comprising administering to the subject a therapeutically effective amount of a plurality of the artificial immunoregulatory T (airT) cells, wherein said airT cells express at least one T cell receptor (TCR) or chimeric antigen receptor (CAR) that specifically recognizes the antigen for which antigen-specific immunosuppression is needed. In some embodiments the condition in need of antigen-specific immunosuppression is an autoimmune condition, an alloimmune condition, an allergic condition, or an inflammatory condition. In some embodiments, an autoimmune disease is a condition in which an immune response targets healthy cells, tissues, and/or organs, causing immune-associated pathology. In some embodiments, an autoimmune disease is immune-associated pathology resulting from dysregulation of the immune response. In some embodiments (i) the autoimmune condition is selected from type 1 diabetes mellitus, multiple sclerosis, systemic lupus erythematosus, myasthenia gravis, rheumatoid arthritis, early onset rheumatoid arthritis, ankylosing spondylitis, immune-mediated pregnancy loss, immune-mediated recurrent pregnancy loss, dermatomyositis, psoriatic arthritis, Crohn's disease, inflammatory bowel disease (IBD), ulcerative colitis, bullous pemphigoid, pemphigus vulgaris, autoimmune hepatitis, psoriasis, Sjogren's syndrome, celiac disease; (ii) the allergic condition is selected from allergic asthma, atopic dermatitis, pollen allergy, food allergy, drug hypersensitivity, contact dermatitis; and (iii) the inflammatory condition is selected from pancreatic islet cell transplantation, asthma, steroid-resistant asthma, hepatitis, traumatic brain injury, primary sclerosing cholangitis, primary biliary cholangitis, polymyositis, stroke, Still's disease, acute respiratory distress syndrome, uveitis, inflammatory bowel disease (IBD), ulcerative colitis, graft-versus-host disease (GvHD), tolerance induction for transplantation, transplant rejection, or sepsis. In some embodiments, the inflammatory condition is primary biliary cholangitis. In some embodiments, the inflammatory condition is primary sclerosing cholangitis. In some embodiments, the inflammatory condition is autoimmune hepatitis. In some embodiments, the autoimmune condition is type 1 diabetes. In some embodiments, the inflammatory condition is islet cell transplantation. In some embodiments, the inflammatory condition is transplant rejection. In some embodiments, the autoimmune condition is multiple sclerosis. In some embodiments, the inflammatory condition is inflammatory bowel disease. In some embodiments, the inflammatory condition is acute respiratory distress syndrome. In some embodiments, the inflammatory condition is stroke. In some embodiments, the inflammatory condition is graft-versus-host disease. In some embodiments (i) the antigen associated with pathogenesis of the autoimmune condition is selected from an autoantigen set forth in any one or more of FIGS. 141-144, (ii) the antigen associated with pathogenesis of the allergic condition is selected from an allergenic antigen set forth in any one or more of FIGS. 141-144, and (iii) the antigen associated with pathogenesis of the inflammatory condition is selected from an inflammation-associated antigen set forth in any one or more of FIGS. 141-144.

Some embodiments of the methods and compositions provided herein include a method for treating or ameliorating a subject having a disorder comprising administering to the subject any one of the foregoing artificial immunoregulatory T (airT) cells. In some embodiments, a method for treating or ameliorating further comprise administering a CISC inducer molecule (e.g., rapamycin or a rapalog) to the subject before or after administering the airT cells to promote proliferation, activation, and/or maintenance of the airT cells in vivo.

In some embodiments, the disorder is selected from the group consisting of an autoimmune disease (e.g., diabetes such as type-1 diabetes, primary biliary cholangitis), autoinflammatory disease (e.g., ARDS, stroke, and atherosclerotic cardiovascular disease), alloimmune disease (e.g., graft-versus-host disease, sold organ transplant, and immune mediated recurrent pregnancy loss), and/or allergic disease (e.g., asthma, drug hypersensitivity, and celiac disease). In some embodiments, a condition to be treated is a cancer. Wang et al. (J Intern Med. 2015 October;278(4):369-95) provide a review of autoimmune diseases, which review is incorporated herein by reference. In some embodiments, the disorder is selected from the group consisting of type 1 diabetes mellitus, multiple sclerosis, systemic lupus erythematosus, myasthenia gravis, rheumatoid arthritis, early onset rheumatoid arthritis, ankylosing spondylitis, immune-mediated pregnancy loss, immune-mediated recurrent pregnancy loss, dermatomyositis, psoriatic arthritis, Crohn's disease, inflammatory bowel disease (IBD), ulcerative colitis, bullous pemphigoid, pemphigus vulgaris, autoimmune hepatitis, psoriasis, Sjogren's syndrome, celiac disease, allergic asthma, atopic dermatitis, pollen allergy, food allergy, drug hypersensitivity, contact dermatitis, pancreatic islet cell transplantation, asthma, steroid-resistant asthma, hepatitis, traumatic brain injury, primary sclerosing cholangitis, primary biliary cholangitis, polymyositis, stroke, Still's disease, acute respiratory distress syndrome, uveitis, inflammatory bowel disease (IBD), ulcerative colitis, graft-versus-host disease (GvHD), tolerance induction for transplantation, transplant rejection, or sepsis. In some embodiments, the TCR polypeptide binds to an antigen associated with a disorder selected from type 1 diabetes mellitus, multiple sclerosis, myocarditis, rheumatoid arthritis (RA), or systemic lupus erythematosus (SLE).

In some embodiments, the TCR polypeptide binds to an antigen selected from the group consisting of vimentin, aggrecan, cartilage intermediate layer protein (CILP), preproinsulin, islet-specific glucose-6-phosphatase catalytic subunit-related protein (IGRP), and enolase. In some embodiments, the TCR polypeptide binds to an antigen comprising an epitope having the amino acid sequence of any one of SEQ ID NOs 1363-1376 and 1408-1415. In some embodiments, the TCR polypeptide binds to an antigen present in or derived from a microorganism present in the gut. In some embodiments, the TCR polypeptide binds to an epitope of the bacterial protein OmpC. In some embodiments, the TCR polypeptide binds to an epitope of a T1D antigen, .e.g., GAD65, PPI, or ZNT8. In some embodiments, the TCR polypeptide binds to an epitope of a PBC antigen, e.g., the E2 component of pyruvate dehydrogenase complex (PDC-E2). In some embodiments, the TCR polypeptide binds to a nuclear antigen. In some embodiments, the TCR polypeptide binds to a mitochondrial antigen.

In some embodiments, the TCR polypeptide comprises: a CD3 alpha polypeptide having the amino acid sequence of any one of SEQ ID NOs 1377-1390; and/or a CD3 beta polypeptide having the amino acid sequence of any one of SEQ ID NOs 1377-1390.

### BRIEF DESCRIPTION OF THE DRAWINGS

FIGS. 1A-11 relate to the engineering of human CD4+ T cells into airT cells using gene editing.

FIG. 1A, FIG. 1B and FIG. 1C depict exemplary schema for converting CD4+ T cells into airT cells of the present disclosure. FIG. 1A is a schematic diagram of FOXP3 locus before (top) and after (bottom) gene editing using FOXP3 TALEN or CRISPR/Cas9 with FOXP3 guide RNA. TALEN or CRISPR/Cas9 cleaves FoxP3 locus at exon 1, initiating site-specific double stranded DNA break. AAV provides donor template containing MND and GFP (to allow analysis of editing efficiency), which is inserted into exon 1 at the DNA break. After the homology-directed repair, the MND promoter drives expression of FoxP3 and GFP reporter. FIG. 1B depicts a timeline of steps of gene editing and cell analysis and efficacy of airT generation from input Tconv cells. FIG. 1C depicts representative flow plots showing correlation between Foxp3 and GFP on day 4 after editing. The three panels on the right-hand side of the figure show CD25, CD127, Helios, CD45RO, ICOS, and CTLA-4 expression in Foxp3+ GFP+ gated cells, respectively.

FIG. 2 depicts flow plots (bottom) showing GFP and Foxp3 expression on day 4 and day 11 after editing according to the timeline shown at top. These data show that Foxp3 editing in CD4+ T cells is efficient and results in high, stable expression of Foxp3.

FIG. 3A, FIG. 3B, FIG. 3C and FIG. 3D depict data comparing airT cells and activated natural T regulatory (nTreg) cells. FIG. 3A depicts a timeline of steps to generate edTreg and activated nTreg for comparison. CD4+ cells were isolated from PBMC using MACS CD4+ isolation kit and Tconv (CD25− CD127+) and Treg (CD25 high CD127−) cells were further sorted by flow. Sorted Tconv and Treg cells were activated with CD3/CD28 activator beads and beads were removed after 48 hr activation. Only Tconv cells were Foxp3-edited using Cas9/Foxp3 gRNA and AAV-MND-LNGFR-Foxp3 ki to generate edTreg/airT. nTreg cells were treated in the same manner without Foxp3 editing. LNGFR+ cells from Foxp3-edited Tconv cells were enriched using MACS LNGFR beads on day 10. LNGFR+edTreg and nTreg cells were used for suppression assay. FIG. 3B depicts a comparison of efficacy in generation of edTreg and nTreg from 1×10⁷ PBMC. At day 0, 1×10⁷ PBMC. Tconv and nTreg cells activated on day 0 were expanded 10-30 times and 1-2 times, respectively, from day 0 to day 10. For edTreg, Treg yield on day 10 was calculated based on editing rate (10-30%). FIG. 3C depicts representative flow plots showing Treg phenotype in Foxp3-edited Tconv and nTreg cells on day 10. Top panels show (left-most panel) LNGFR expression in edited Tconv and (right) Foxp3, Helios, CD25, CD127, ICOS, and CTLA-4 expression in edited Treg (LNGFR+ gate, top panels) and nTreg (bottom panels). FIG. 3D (upper panels) depicts comparison of Foxp3, CTLA-4, and ICOS expression in edTreg/airT and nTreg. FIG. 3D (bottom table) shows the MFI.

FIG. 4A and FIG. 4B show that airT cells have superior in vitro suppressive activity to nTreg. FIG. 4A depicts data from an in vitro suppression assay comparing suppressive activities of edTreg/airT and nTreg on CD4+ T_{eff} cells at the indicated Treg:T_{eff} ratios. airT or nTreg cells were labeled with EF670, and CD4+ T_{eff} cells were labeled with Cell Trace Violet (CTV). T_{eff} cells were co-cultured with airT or nTreg at different ratios, 0:1 (T_{eff} only), 1:1, 1:2, 1:4, 1:8, 1:16, and 1:32 (Treg:T_{eff}). CD3/CD28 activator beads were added at 1:25 (bead to T_{eff} ratio) and cells were analyzed by flow after 4d incubation. Dilution of CTV in T_{eff} cells was measured as proliferation. FIG. 4B depicts percent suppression calculated as (% proliferation in T_{eff} only+beads−% proliferation in T_{eff} cells cultured with Treg)/(% proliferation in T_{eff} only+beads)×100.

FIG. 5 depicts exemplary lentiviral islet-specific TCR constructs expressing rare islet-specific TCRs derived from Type 1 diabetes (T1D) subjects. Panel A depicts a table of lentiviral vectors encoding GAD65 or IGRP specific TCRs (4.13, T1D2, T1D4, T1D5-1, or T1D5-2), their epitope specificity, and TCR alpha or beta chain usage. Panel B depicts structure of lentiviral islet-specific TCR. TCR constructs include human TCR variable regions from the islet-specific TCRs and mouse TCR constant regions that allow to improve pairing between the transduced human TCR chains.

FIG. 6 depicts validation of islet Ag-specific TCR expression: murine TCRβ expression and proliferation of islet antigen-specific T cells. Panel A depicts flow plots for CD4+ T cells isolated, activated with CD3/CD28 beads, and transduced with LV islet-TCRs. Flow plots show mTCRβ expression gated on CD3/CD28-activated CD4+ cells day 9 post-transduction with lentivirus (LV) encoding islet-specific TCR. Panel B depicts flow plots for CD4+ T cells transduced with LV islet-TCRs labeled with CTV and co-cultured with APC (irradiated PBMC) and their cognate peptide or irrelevant peptide for 5 days. Flow plots showing cell proliferation of LV-transduced CD4+ T cells labeled with CTV following 5-day co-culture with antigen-presenting cells (APC; irradiated PBMC) and cognate or irrelevant peptide. Proliferation is shown as CTV dilution.

FIG. 7 shows generation of Foxp3-edited T cells with islet-specific TCR. Panel A depicts a timeline of generating edTreg cells with islet-specific TCRs. Panel B depicts representative flow plots showing mTCRβ expression and LNGFR/Foxp3 expression on CD4+ cells on day 7 after transduction with T1D4 or T1D5-1 TCR and Foxp3 editing. Right panels show expression of CD25, CD127, CTLA-4, and ICOS gated on LNGFR+ cells.

FIG. 8 relates to exemplary antigen-specific suppression assays of the present disclosure. Panel A depicts a timeline for generation of edTreg cells expressing islet-specific TCRs. edTreg cells with islet-specific TCRs (no LV TCR, T1D4, or T1D5-1 TCR) were enriched by LNGFR expression using MACS LNGFR beads. LNGFR+ cells were aliquoted and frozen down for further experiments. Panel B depicts a summary of method used to assess antigen-specific suppression assays. CD4+ T cells transduced with islet-specific TCRs (T1D4 or T1D5-1 TCR) were used as T_{eff} cells. T_{eff} cells and Treg cells were labeled with different reagents, for example CTV or EF670, and co-cultured with or without edTreg cells with 1:1 or 1:2 ratio in the presence of APC (autologous irradiated PBMC) and various peptides. Cells were stained and analyzed by flow after 1 d or 4 d incubation for measuring cytokine generation and proliferation of T_{eff} cells, respectively.

FIG. 9 and FIG. 10 depict suppressive activity of edTreg/airT on T_{eff} proliferation in the presence of APC and the indicated peptide(s). T_{eff} and Treg cells were labeled with CTV and EF670, respectively. CD4+ T cells transduced with T1D4-TCR (T1D4 T_{eff}) were co-cultured with or without edTreg expressing T1D4-TCR (T1D4 edTreg) or T1D5-1-TCR (T1D5-1 edTreg) in the presence of APC and various peptides (DMSO, IGRP 241, IGRP 305, or IGRP241+IGRP 305). 4 days after the co-culture, cells were stained and analyzed for T_{eff} proliferation as dilution of CTV. Flow plots show T_{eff} proliferation gated on CD3+ CD4+ CTV+ EF670− LNGFR−.

FIG. 11 depicts suppression of cytokine generation in T_{eff} by edTreg/airT. T_{eff} and Treg cells were labeled with CTV and EF670, respectively. T1D4 T_{eff} cells were cocultured with or without untransduced edTreg or T1D4 edTreg/airT cells in the presence of APC and peptides (DMSO or IGRP 241). 1 day after the co-culture, cells were contacted with BFA for 4 h, stained, and analyzed for cytokine generation from T_{eff} cells. Flow plots show TNF, IFNg, or IL-17 generation from T1D4 T_{eff} cells gated on CD4+ CTV+ EF670−.

FIGS. 12-17 relate to the development and characterization of antigen-specific human Foxp3-edited human CD4+ T cells.

FIG. 12 depicts (top) an exemplary scheme for generating human antigen-specific edTreg/airT from peripheral blood cells and (bottom) phenotype of FOXP3-edited human antigen-specific CD4+ T cells. In the bottom panels, representative flow plots (left) and percentage (right) of GFP expression in tetramer positive (Tr+; a mixture of MHC class II tetramers with flu or tetanus peptides) human CD4+ T cells at 4 days post-gene editing (n=5).

FIG. 13 depicts a characterization of FOXP3-edited human antigen-specific CD4+ T cells. Panel A depicts phenotype of FOXP3 edited human antigen-specific CD4+ T cells. Bar chart summarizes flow cytometry data (n=5); chart shows expression of Treg markers and intracellular IL-2 production in Tmr+edTreg, Tmr+ Mock-edited cells, as well as in thymus-generated Treg (tTreg) obtained from an unrelated donor. Data shown are representative of 5 independent experiments. P values of statistically significant differences are indicated above bars. Panel B depicts human antigen-specific edTreg/airT suppresses proliferation of T_{eff} in vitro. Suppression assays conducted using Tmr+edTreg/airT or mock-edited Tmr+ cells co-cultured with T_{eff} from healthy controls, APCs, and soluble anti-CD3 and anti-CD28. Ratio of antigen presenting cells (irradiated CD4− PBMC): Tmr+edTreg or mock-edited Tmr+ cells: T_{eff} was 2:1:1. 1 μCi ³H was added 18 hours prior to the end of the 4 day assay and proliferation was measured by a scintillation counter. Bar graph indicated averaged results from three experiments with three donors.

FIG. 14 depicts successful generation of antigen-specific edTreg/airT by peptide stimulation followed by Foxp3 editing. Panel A depicts a timeline of steps of antigen-specific T cell expansion and gene editing. After 9 days of peptide stimulation to expand T cells specific for MP, HA, or Tetanus, cells were activated with CD3/CD28 activator beads for gene editing. Beads were added to the sorted cells to enhance expansion of antigen-specific Tregs. Panel B depicts flow plots show GFP and Foxp3 expression on day 15 after editing. GFP+ Foxp3+ cells were CD25+ CD127− and about 60% of cells were MP, HA, or TT specific by tetramers.

FIG. 15 depicts antigen-specific suppression by Foxp3-edited Tregs/airT. Panel A a timeline of steps of generating antigen-specific edTreg/airT cells for suppression assay. GFP+ cells were sorted and expanded with CD3/CD28 beads on day 15 after editing. Beads were removed after 7d incubation and edTreg/airT cells were harvested and used for suppression assay after 11 days of expansion. Panel B depicts a summary of suppression assay design. CD4+CD25+ cells were isolated from autologous PBMC, labeled with EF670, and used as T_{eff} cells. CD4−CD25+ cells were irradiated and used as APC, and edTreg/airT cells were labeled with Cell Trace Violet (CTV). T_{eff} cells and APC were co-cultured with or without edTreg/airT cells in the presence of DMSO or peptide pool (MP+HA+TT). Panel C depicts after 7 days of co-culture, cells were stained and analyzed by flow. CD3+CD4+EF670+ CTV− cells were gated as T_{eff} cells. Panel D depict a dilution of EF670 in T_{eff} cells was measured as proliferation and 15% of EF670− cells from co-culture of T_{eff} cells with APC and the peptide pool was normalized as 100% proliferation. % suppression was calculated as (100-% Proliferation).

FIG. 16 depicts an expansion of islet-specific T cells of multiple specificities by peptide stimulation. Panel A depicts an exemplary timeline for generating islet-antigen specific edTreg/airT cells. Freshly isolated CD4+CD25− cells were stimulated by a pool of islet-specific peptides and APC (irradiated autologous CD4− CD25+ cells) for 14 days and expansion of islet-specific T cells was analyzed on day 13 by tetramer staining. Panel B depicts flow plots showing islet-specific T cells stained by individual tetramers or tetramer pool, gated on CD4+ cells.

FIG. 17 depicts generation of islet-specific Tregs of multiple specificities. Panel A depicts islet-specific T cells were stained by tetramers and sorted on day 14. Sorted tetramer+ cells were activated with CD3/CD28 beads for 72 h for Foxp3 editing. 3 days after editing, cells were stained and analyzed. Flow plots show Foxp3 and LNGFR expression in mock or edited cells (left) and CD25, CD127, and CD45RO expression in LNGFR+ gated cells (right). Panel B depicts cells were stained by individual tetramers or tetramer pool and flow plots show tetramer+ cells in LNGFR+ Foxp3+ edited cells.

FIGS. 18-33 relate to the generation of dual-edited human CD4+ T cells using bi-allelic targeting to engineer artificial Treg cells expressing Foxp3 and antigen-specific TCR, with endogenous TCR inactivation.

FIG. 18 depicts a schematic of an exemplary CD4+ T cell edited to possess Treg phenotype and to express exogenous Ag-specific TCR, but not endogenous TCR. In this scheme, the conversion of a conventional CD4+ T-cell into an antigen-specific Treg comprises three genetic alterations: 1) stable expression of the transcription factor FOXP3 to drive cells toward a Treg phenotype; 2) stable expression of a defined, antigen-specific rearranged T-Cell receptor (Ag-specific TCR) to direct Treg immunosuppressive activity; and 3) genetic deletion of the endogenous T-Cell Receptor (TCR) to ensure that immunosuppressive function is directed solely toward the desired antigen.

FIG. 19 depicts exemplary AAV constructs for CRISPR gene editing at the human and mouse TRAC loci. The list includes adeno-associated virus plasmid constructs generated for CRISPR-based homology directed repair, organized based on the relevant gRNA, and includes number designation.

FIG. 20 depicts an exemplary CRISPR-based approach for targeting of the human TRAC locus for knockout/knock-in. In particular, the image shows a schematic representation of the human TRAC locus showing the relative position of the four gRNA sequences tested (PC_TRAC_E1_gRNA1 to PC_TRAC_E1_gRNA4). The TRAC exon 1 is indicated by the lowermost bar from about position 1160 continuing past 1400. Common SNPs are indicated by about positions 1160 and 1400. The position of a previously published positive control gRNA sequence (TCRa G4old) is indicated at about position 1320.

FIG. 21 relates to guide RNA (gRNA) qualification of non-homologous end joining (NHEJ) for knockout of CD3 in human CD4+ primary T cells. Data are from FACS analysis. Panel A depicts flow plots show expression of CD3 2 days post-editing in mock-edited and TCR-edited CD4+ T cells using four different guide RNAs. TCRa_G4old, previously demonstrated to knockout CD3 expression, was used as a control. Panel B depicts histograms showing percent CD3 knockout.

FIG. 22 depicts results from Inference of CRISPR Edits (ICE) analysis of indel frequency. On-target site-specific activity was measured by ICE (Inference of CRISPR Edits) and confirmed specific indel induction for gRNA_1 and gRNA_4 in TRAC relative to predicted off-target sites.

FIG. 23 depicts results from ICE analysis of predicted off target sites for TRAC gRNAs. The top 3 predicted off target sites for TRAC gRNA 1 and TRAC gRNA 2 (based on frequency and position of mismatches) were tested for indel induction frequency by ICE sequence deconvolution analysis.

FIG. 24 depicts an exemplary experimental outline for performing dual AAV editing for assessment of bi-allelic knock-in. A. Diagram of AAV constructs used in this experiment; after editing, MND promoter drives expression of GFP/BFP. B. Timeline of experimental procedures. CD4+ T cells were bead-stimulated (CD3/CD28) for 3 days prior to editing. Three and six days post-editing, cells were evaluated for GFP and BFP expression by flow cytometry.

FIG. 25 depicts dual editing of the TRAC locus in human CD4+ cells leads to a double-positive population of cells. Panel A depicts flow plots show GFP and BFP expression in mock-edited, and mixed MND.GFP- and MND.BFP-edited cells (10% #3207 virus+10% #3208 AAV) two days post-editing. Viral titers were 3.3×10¹² and 2.53×10¹² for #3207 and #3208, respectively. Panel B depicts histograms showing percent double-negative, GFP single-positive, mCherry single-positive and GFP/mCherry double-positive cells within the dual-edited cells.

FIG. 26 depicts schematic diagrams showing exemplary Split IL-2 CISC HDR knock-in constructs for selection of dual-edited cells. In the depicted constructs, CISC (chemically induced signaling complex) is split onto 2 different constructs and each CISC component is co-expressed with a different reporter, in this case either GFP or mCherry. Each construct contains half of a rapamycin-binding complex (either FKBP or FRB domain, with the chimeric endoplasmic reticulum targeting domain fused to one half of an IL-2R signaling complex (IL-2RB or IL-2RG) transmembrane and intracellular domains. Delivery of cDNA encoding each CISC component co-expressed with the GFP/mCherry tag to primary human CD4+ T cells allows selective expansion of cells that contain both CISC components and thus are also dual edited for GFP and BFP.

FIG. 27 depicts an exemplary timeline of steps for dual AAV editing of CD4+ T cells, expansion with rapalog, and analysis of enriched cells. Cells were bead stimulated (CD3/CD28) for 3 days prior to editing. Two days post-editing, cells were analyzed by flow for GFP and mCherry expression, and then expanded in media containing 50 ng/ml human IL-2 or 100 nM rapalog. Flow cytometry to assess enrichment of GFP, mCherry double-positive cells was carried out on days 6, 8, and 10 post-editing.

FIG. 28 depicts FACS analysis of initial dual editing rate. Panel A depicts flow plots show GFP and mCherry expression in mock-edited, MND.GFP.FRB.IL-2RB-edited (20% #3207 AAV), MND.mCherry.FKBP.IL-2RB (20% #3208 AAV)-edited and mix-edited (10% #3207+10% #3208) cells. Viral titers were 3.3×10¹² and 2.53×10¹² for #3207 and #3208, respectively. Panel B depicts histograms show percent of double-negative, GFP-positive, mCherry-positive and GFP/mCherry double-positive cells within the dual-edited cells.

FIG. 29 depicts exemplary data showing rapalog enrichment of dual-edited cells. Panel A depicts flow plots show GFP and BFP expression in mock-edited, and mixed MND.GFP- and MND.BFP-edited cells (10% #3207 virus+10% #3208 AAV) two days post-editing. Viral titers were 3.3×10¹² and 2.53×10¹² for #3207 and #3208, respectively. Panel B histograms showing percent double-negative, GFP single-positive, mCherry-single positive and GFP/mCherry double-positive cells within the dual-edited cells.

FIG. 30 depicts histograms showing percent double-negative, GFP single-positive, and mCherry single-positive cells after contact with IL-2 and rapalog. These data show that single-positive and unedited populations do not significantly change with rapalog treatment.

FIG. 31 depicts data from FACS analysis of initial dual editing rates using two different donors. Panel A depicts a timeline of editing and analysis steps. Panel B depicts histograms showing percent double-negative, GFP-positive, mCherry-positive and GFP/mCherry double-positive cells within the dual-edited cells for each donor. Donor R003657 is male, Caucasian and 28 y.o. Donor R003471 is male, Caucasian and 29 years old.

FIG. 32 depicts data from FACS analysis of rapalog enrichment of Bi-Allelic R003471 cells. Panel A depicts flow plots showing expression of GFP and mCherry following 5 days enrichment in rapalog. Panel B depicts histograms showing percent GFP/mCherry double-positive cells after expansion in IL-2 or rapalog.

FIG. 33 depicts schematic diagrams showing exemplary split-CISC constructs for insertion of TCR and Foxp3 and enrichment of dual edited cells. CISC is split onto two different constructs and each CISC component is co-expressed with either an Ag-specific TCR (in the diagram, exemplary T1D4 TCR) or Foxp3. Each construct contains half of a rapamycin-binding complex (either FKBP or FRB domain, with the chimeric endoplasmic reticulum targeting domain fused to one half of an IL-2R signaling complex (IL-2RB or IL-2RG) transmembrane and intracellular domains. Delivery of cDNA encoding each CISC component co-expressed with the T1D4 TCR/Foxp3 to primary human CD4+ T cells allows selective expansion of cells that contain both CISC components and thus are also dual edited for T1D4 TCR and Foxp3.

FIGS. 34-37 relate to the generation of reagents for assessing antigen-specific airT cell function in in vivo models of autoimmunity.

FIG. 34 depicts a schematic representation of the murine TRAC locus showing the relative position of the three novel gRNA sequences tested (PC_mmTrac_E1_gRNA1 to PC_mmTrac_E1_gRNA3). The TRAC exon 1 is indicated.

FIG. 35 depicts data from FACS analysis of CD3 knockout in murine CD4+ T cells. Panel A depicts flow plots show expression of murine CD3 two days post-editing in mock-edited and TCR-edited CD4+ T cells using three different guides. Panel B depicts histograms showing percent mCD3 knockout for each guide RNA.

FIG. 36 depicts an exemplary experimental outline for dual AAV editing for assessment of bi-allelic knock-in. Panel A depicts a diagram of AAV constructs used in this experiment; after editing, MND promoter drives expression of GFP/BFP. B. Timeline of experimental procedures. Murine CD4+ T cells were bead stimulated (CD3/CD28) for 3 days prior to editing. Three and five days post-editing, cells were evaluated for GFP and BFP expression by flow cytometry.

FIG. 37 depicts data from FACS analysis of single- and dual-editing rates in the murine TCRα locus. Flow plots show GFP and BFP expression 3 days post-editing in mock, MND.GFP (10% #3211), MND.BFP (10% #3212), and mix-edited cells (5% #3207+5% #3208). Mixed edited cells had a total of 1.97% GFP/BFP double-positive cells.

FIGS. 38-43 relate to airT cell function in an antigen-specific in vivo setting.

FIG. 38 depicts a schematic diagram of an experimental design to test the ability of MOG-specific edTreg/airT cells (shown in white) to suppress T effectors (T_{eff}) cells (shown in gray) in a mouse model of multiple sclerosis, Experimental Autoimmune Encephalomyelitis.

FIG. 39 relates experiments showing that mouse FOXP3 TALENs catalyze efficient FOXP3 disruption and initiate non-disruptive recombination of donor template. Panel A depicts binding sites for the FOXP3 TALEN pair in the human FOXP3 gene. Panel B depicts target binding sites for the mouse FOXP3 TALEN pair in the murine FOXP3 gene. Panel C depicts indel frequency at FOXP3 TALEN cut site in human (left) and mouse (right) CD4+ T cells 5˜7 days after transfection with mRNA encoding either control mRNA (encoding blue fluorescent protein), or TALENs specific for human FOXP3 or mouse FoxP3, respectively. Graph shows average frequency of indels after colony sequencing PCR amplicons surrounding gDNA target site; 20-40 colonies were sequenced per experiment.

FIG. 40 relates to generation of edTreg/airT from antigen-specific murine CD4+ T cells. Panel A depicts a schematic diagram of FOXP3 locus after successful gene editing using mouse FOXP3 TALENs and the mouse AAV FOXP3 MND-GFP knock-in (ki) donor template. After editing, the MND promoter drives expression of chimeric GFP-FoxP3 protein. Panel B depicts flow plots showing GFP expression in antigen-specific mouse CD4+ T cells at Day 2 post-editing. Panel C depicts average percent of GFP+ cells across multiple experiments (n=10). D. Flow plot of murine edTreg/airT showing expression of relevant Treg markers.

FIG. 41 shows functional assessment of antigen specific vs. polyclonal edTreg/airT in a mouse model of Multiple sclerosis. Panel A depicts flow plots showing GFP expression in MOG-specific and polyclonal mouse CD4+ T cells at Day 2 post-editing after FACS sorting. Panel B depicts schematic diagram of murine EAE in vivo experimental design and timeline. 2D2 (MOG-specific) T_{eff} (30K) were delivered with or without co-transferred edTreg/airT (30K) generated from either 2D2 or C57B1/6 mice into RAG1−/− recipient mice; all strains were on C57B1/6 background. Analysis was performed at Day 7.

FIG. 42 depicts data showing that antigen-specific edTreg/airT delay expansion, activation and cytokine production of T_{eff}. Immunophenotype of T cells obtained from inguinal and axillary lymph nodes in recipient mice at day 7 post-cell transfer was assessed by flow cytometry. CD45+=panCD45 (recognizing all CD45 isoforms and both CD45.1 and CD45.2 alloantigens). Shown are total number of total CD45+ CD4+ cells (A) and other indicated T cell subsets (B) and (C), expansion of GFP+ cells. Data is representative of results from 3 independent experiments; bar graphs show mean±SD; p-values of statistically significant differences are indicated above bars.

FIG. 43 provides data showing that antigen-specific edTreg/airT cells suppress T_{eff} proliferation in vivo. Panel A depicts flow plots: to label actively dividing cells, the thymidine analog 5-Ethynyl-2′-deoxyuridine (EdU) was administered 2 hours prior to sacrifice in selected animals. EdU incorporation in T cells was determined by intracellular labeling with an anti-EdU antibody and flow cytometry. Flow plots are from T cells isolated from LNs 7 days post-cell transfer. Panel B depicts bar graphs summarize mean % of cells incorporating EdU in different cell subsets and (C) the % GFP+lymphocytes. Flow plots are representative of results from at least 3 independent experiments; bar graphs show mean±SD; p-values of statistically significant differences are indicated above bars.

FIGS. 44-47 relate to experiments investigating antigen specific T cell function in a NSG adoptive transfer model of Type 1 diabetes. Engineered antigen-specific (BDC) or polyclonal (NOD) edTregs/airTs, or antigen-specific nTregs were infused into the mice followed by infusion of antigen-specific T_{eff} cells. Mice were monitored for diabetes up to 90 days following infusion. Graph shows the percent of diabetic mice that received effector cells plus the designated mock edited, Foxp3-edited, or nTreg cells from NOD and BDC2.5 mice.

FIG. 44 relates to Foxp3 editing in CD4+ T cells of antigen-specific NOD mice. Panel A depicts CAS9/CRISPR RNP cutting efficiency in BDC2.5 NOD mice using different guide RNAs. Panel B depicts AAV5-delivered repair template. After editing, the MND promoter will drive expression of chimeric GFP-FoxP3 protein. Panel C depicts flow plots showing GFP expression in mock-edited and GFP-Foxp3-edited antigen-specific mouse CD4+ T cells at day 2 post-editing.

FIG. 45 relates to phenotype of FOXP3-edited antigen-specific NOD CD4+ T cells. Left. Flow cytometry plots showing GFP and Foxp3 expression in edited cells. Middle. Flow cytometry plots showing IL-2, IFN-g and IL-4 expression in GFP-Foxp3-edited (upper plots) and mock-edited (lower plots) murine antigen-specific NOD CD4+ T cells. Right. Histograms showing % of cells positive for IL-2, IFN-γ and IL-4 four days post-editing.

FIG. 46 relates to an experiment investigating phenotype of input cells for NSG adoptive transfer model. Panel A depicts an experimental design showing amount and type of cells administered for each group of animals. Panel B depicts flow cytometry plots showing the phenotype of T_{eff}, edTreg/airT and nTreg cells injected into NSG mice.

FIG. 47 relates to antigen-specific T cell function in NSG adoptive transfer model. Panel A depicts an experimental design; engineered antigen-specific (BDC) or polyclonal (NOD) edTregs/airTs, or antigen-specific nTregs were infused into the mice, followed by infusion of antigen-specific T_{eff} cells. Mice were monitored for diabetes up to 90 days following infusion. Panel B depicts a graph shows the percent of diabetic mice that received effector cells plus the designated mock-edited, Foxp3-edited, or nTreg cells from NOD and BDC2.5 mice. Antigen-specific edTreg/airT exhibited significantly greater level of protection from T1D compared with mock-edited T cells, polyclonal edTregs/airTs or polyclonal nTregs.

FIGS. 48-51 relate to engineering a mouse AAV donor template design to generate airT cell product with a selectable marker (LNGFR).

FIG. 48 depicts exemplary repair templates used in murine Foxp3 editing. AAV.Promoter-LNGF.P2A knock-in constructs were tested in murine T cells for stable expression of Foxp3.

FIG. 49 depicts phenotype of murine edTreg/airT using alternative homology donor cassettes. Flow cytometry plots show LNGFR, FOXP3, CD25, and CTLA-4 in mock-edited cells and cells edited with MND.LNGFR.P2A KI (3189) or PGK.LNGFR.P2A KI (3227).

FIG. 50 depicts data showing editing rate and expression of LNGFR in murine edited Treg/airT cells. Flow cytometry plots show LNGFR and GFP expression in mock, MND-GFPki (#1331) MND.LNGFR.P2A.KI (#3189) edited cells.

FIG. 51 depicts data showing enrichment of LNGFR+ edited T cells from B6 mice using an anti-LNGFR column. Flow cytometry plots show LNGFR expression of cells prior to purification on a Miltenyi anti-LNGFR column, cells in the flow through and cells eluted from the column.

FIG. 52 depicts a comparison of FOXP3-edited vs. FOXP3 lentiviral (LV) transduced human CD4 T cells. Panel A depicts a diagram of LV construct: MND promoter drives expression of a transcript encoding identical GFP-FOXP3 fusion protein as that of airT; transcript contains WPRE and poly(A) signals for efficient nuclear export and mRNA stability. Below are representative flow plots showing FOXP3 and GFP expression in mock-edited T cells or sorted tTreg (CD4+CD25++CD127−), airT and LV Treg (CD4+GFP+), all post ≥14-day expansion in vitro with CD3/CD28 beads. Panel B depicts mean viral copy number (±SD) of LV-transduced sorted cells (left; n=6). Scatter plots (right) show the MFI of the GFP+ population for each sample (n=5; P value from two-tailed Student's T-test). Panel C depicts bar graphs showing mean % of cells (top), and MFI (bottom) by flow cytometry staining for the proteins indicated. Viable singlets were further gated on: CD4+ GFP+ (LV Treg and edTreg), CD4+FOXP3+ (tTreg), or CD4+ (mock). For markers with distinct bimodal distributions, MFI was calculated for the positive population only. Error bars show±SD. An ordinary two-way ANOVA was performed, and P values adjusted with Tukey's multiple comparisons test. P values in black indicate comparison with mock-edited cells; those in red were comparison of groups indicated by dashed lines. Panel D depicts percent suppression as a function of Treg or mock dilution (top). Histograms of proliferation dye at different ratios of Treg or mock to T_{eff} (bottom). % suppression=[(% divided with no Treg−% divided with Treg)/% divided with no Treg]×100. Panel E depicts a plot showing data points and simple linear regression of % GFP+ cells over time in culture after FACS purification; airT (n=4) and LV Treg (n=6); data from 6 experiments. Dashed lines indicate 95% confidence intervals; P value was obtained using an F Test.

FIGS. 53-73 provide additional schematics and data related to exemplary dual-editing strategies of the present disclosure for generation of antigen-specific, drug-selectable airT cells with knock-out of endogenous TCR.

FIG. 53 depicts schematics showing dual-editing strategies designed to: a) eliminate the endogenous TCR expression and b) generate selectable antigen-specific airTs. Delivery of expression cassettes for FOXP3 and a candidate islet antigen-specific TCR (T1D4) paired to the two halves of the IL-2 CISC/DISC (FKBP-IL2RG and FRB-IL2RB), can be directed to the same locus (Strategy 1) or two separate loci (Strategy 2). Targeting of the TRAC locus in CD4+ T cells allows for deletion of the endogenous TCR. Strategy 2 may result in higher initial dual editing rates but requires two nuclease target sites, leading to two double stranded breaks (DSBs) in the host cell genome that mediate HDR. Strategy 1 utilizes a single nuclease target site leading to a single DSB.

FIG. 54 depicts a schematic of AAV HDR donor constructs used in human T cell dual-editing. The first 7 constructs are IL-2 split-CISC repair templates with either GFP, mCherry, HA-tagged FOXP3 or T1D4 driven by the MND promoter. Each component of the split CISC includes a heterodimeric rapamycin binding complex (either FKBP and FRB domains), along with the chimeric endoplasmic reticulum targeting domain fused to one half of the IL2R signaling complex (either IL2RB or IL2RG) trans-membrane and intracellular domains. Each repair template is flanked by 300 bp homology arms matched to a gRNA targeting either the TRAC locus (gRNA_4) or FOXP3 locus (gRNA_T9) (#3207, 3208, 3240, 3243, 3251, 3252, 3273). The next four constructs (#3253, 3258, 3292, 0001) are used for in-frame knock-in of a promoter-less TCR cassette including components of the CISC, targeting the first exon of TRAC locus (gRNA_1). The final two constructs (#3280 and #3262) are split-DISC repair templates that include the CISC elements as well as cDNA encoding a free FRB domain that functions in cytoplasmic Rapamycin sequestration (which eliminates or reduces any negative impact of rapamycin on gene edited cells). These latter constructs also contain either mCherry or FOXP3 driven off the MND promoter.

FIG. 55 depicts dual editing rates within the human TRAC locus in the presence or absence of rapalog-based selection of CISC edited CD4+ T cells from donor R003657. Panel A depicts a timeline of editing (using RNP and AAV co-delivery), enrichment and analysis steps with donor R003657 CD4+ T cells using AAV #3207 and #3208. Panel B depicts flow plots show initial percent GFP/mCherry double positive cells in the mock vs. dual-edited samples, and percent GFP/mCherry double positive following 7 days enrichment in the presence of IL-2 or Rapalog (AP21967). Panel C depicts histograms show percent double-negative, GFP-positive, mCherry-positive and GFP/mCherry double-positive cells within the dual-edited cells following enrichment in IL-2 vs. Rapalog.

FIG. 56 depicts dual editing rates in the TRAC locus and rapalog-based selection of CISC-edited CD4+ T cells from donor R003471. Panel A depicts a timeline of editing, enrichment and analysis steps with donor R003471 CD4+ T cells using AAV #3207 and #3208. Panel B depicts flow plots show initial percent GFP/mCherry double-positive cells in the mock- vs. dual-edited samples, and percent GFP/mCherry double-positive following 7 days enrichment in the presence of IL-2 or Rapalog (AP21967). Panel C depicts histograms show percent double-negative, GFP-positive, mCherry-positive and GFP/mCherry double-positive cells within the dual-edited cells following enrichment in IL-2 vs. Rapalog.

FIG. 57 shows dual-editing of the TRAC locus in human CD4+ T cells generates rapalog-selectable, antigen-specific airTs. Panel A depicts a schematic showing AAV HDR donors construct used to introduce of the “split” IL-2 CISC elements for selection of dual-edited cells. CISC components (IL2RG vs. IL2RB) are split between 2 constructs and co-expressed with either HA-FoxP3 cDNA or the islet-specific TCR, T1D4 (AAVs #3240 and #3243 respectively). Each repair template is flanked by identical homology arms that cannot be cleaved by the gRNA targeting the TRAC locus. Only edited CD4+ T cells incorporating one copy of each construct are predicted to selectively expand under Rapalog treatment. Panel B depicts a timeline of key steps for dual AAV/RNP-based editing of CD4+ T cells, expansion with Rapalog and analysis of enriched cells. Cells were bead stimulated (CD3/CD28) for 3 days prior to editing. Two days post-editing, cells were analyzed by flow for HA-FoxP3 and TCR expression, and then expanded in media containing 50 ng/ml human IL-2 or 100 nM Rapalog. Flow cytometry to assess enrichment of HA-FoxP3, TCR double-positive cells was carried out on days 5 and 8 post-editing. Panel C depicts rapalog enrichment of dual-edited cells. Left panel: Flow plots for HA-FoxP3 and TCR in dual-edited cells following 8 days expansion in IL-2 or Rapalog; Right panel: quantitation of percent HA-FoxP3/TCR double-positive cells following expansion in IL-2 or Rapalog for 5 and 8 days.

FIG. 58 provides data showing that decreasing serum concentration increases total- and dual-editing rates within the TRAC locus. Panel A depict a timeline showing steps for dual AAV editing of CD4+ T cells and expansion with Rapalog. Human CD4+ T cells were edited using TRAC gRNA_4 and #3243 and #3240 AAV constructs (Single-locus dual editing). Immediately following electroporation to deliver the RNP, the cells were placed in either 20%, 2.5%, 1% or 0% FBS containing media (recovery media) and infected with AAV. After ˜16 hours, the media was replaced with 20% FBS containing media and FACS analysis done on day 3 to determine editing rate. Cells recovered in 2.5% FBS containing media were expanded in the presence of either IL-2 or Rapalog for an additional 7 days. Panel B depicts flow plots show T1D4 and FOXP3 expression in mock-edited, single-edited and mixed edited cells (10% #3243 and 10% #3240 AAV) three days post editing. Viral titers were 4.2E¹¹ and 1.3E¹² for #3243 pAAV.MND.T1D4.FRB.IL2RB and #3240 pAAV.MND.FOXP3-HA.FKBP.IL2RG respectively. Panel C depicts histograms show percent double-negative, FOXP3-HA-positive, T1D4-positive and FOXP3/T1D4 double-positive cells within the dual-edited cells.

FIG. 59 shows IL-2 vs. Rapalog enrichment of dual-edited cell populations. TRAC locus dual-editing was performed as shown in FIG. 5. Panel A depicts flow plots show T1D4 and FOXP3 expression in mock-edited vs. FOXP3/T1D4 (#3240/3243) dual-edited cells treated with either 50 ng/mL IL-2 or 100 nM Rapalog (AP21967) for 7 days. Data are shown only for the 2.5% FBS recovery media condition. Panel B depicts histograms show percent double-negative, FOXP3-HA-positive, T1D4-positive and FOXP3/T1D4 double-positive cells within the dual-edited cells following enrichment.

FIG. 60 relates to a strategy for testing two-loci dual-editing of human CD4+ T cells. Panel A depicts a diagram of AAV HDR-donor constructs designed to introduce split IL-2 constructs for selection of dual-edited cells using a two loci dual-editing approach. CISC components are split between 2 constructs and co-expressed with either mCherry or GFP (#3207 and #3251 respectively). Repair templates are flanked by homology arms matched to gRNAs targeting either the TRAC or FOXP3 locus, respectively. Only edited CD4+ T cells that incorporate both expression cassettes (into the appropriate locus) are predicted to selectively expand under Rapalog treatment. Panel B depicts a timeline showing steps for dual AAV editing of CD4+ T cells and expansion with Rapalog. Human CD4+ T cells were edited using human TRAC gRNA_4, human FOXP3 gRNA_T9 and #3251 (MND.mCherry.FKBP.IL2RG) and #3207 (MND.GFP.FRB.IL2RB) AAV constructs (two-loci dual editing). Immediately following electroporation, the cells were placed in either 20% or 2.5% FBS containing media (recovery media). After ˜16 h, the media was replaced with 20% FBS containing media and FACS analysis done on day 3 to determine editing rate. Cells recovered in 2.5% FBS containing medium were further grown in the presence of either IL-2 or Rapalog for an additional 7 days to monitor enrichment.

FIG. 61 shows that recovery in 2.5% FBS containing medium improves dual-editing rates measured at Day 3 post-editing. Two-loci dual editing was performed as shown in FIG. 59. Panel A depicts flow plots show GFP and mCherry expression in mock-edited and dual-edited cells in 20% FBS vs. 2.5% FBS recovery media at 3 days post-editing. Viral titers were 6.55E\hat{ }10 and 2.50E\hat{ }12 for #3251 pAAV.MND.mCherry.FKBP.IL2RG and #3207 pAAV.MND.GFP.FRB.IL2RB respectively, and 10% culture volume of each virus was used for the editing reactions. Panel B depicts histograms show percent double-negative, GFP-positive, mCherry-positive and GFP/mCherry double-positive populations within the dual-edited cells.

FIG. 62 shows robust enrichment of two-loci dual-edited cells treated with rapalog selection. Two-loci dual-editing was performed as shown in FIG. 59. Panel A depicts flow plots show GFP and mCherry expression in mock-edited and GFP/mCherry (#3207/3251) edited cells (edited in 2.5% serum) treated with either 50 ng/mL IL-2 vs. 100 nM Rapalog (AP21967) for 10 days. Panel B depicts histograms show percent double-negative, GFP-positive, mCherry-positive, and GFP/mCherry double-positive cells within the edited population following treatment in IL-2 vs. Rapalog for 10 days.

FIG. 63 relates to engineering of two-loci dual-editing of human CD4+ T cells. Editing conditions and timeline for dual AAV editing of CD4+ T cells and expansion with Rapalog. Human CD4+ T cells were edited using human TRAC gRNA_4, human FOXP3 gRNA_T9 and #3251 (MND.mCherry.FKBP.IL2RG) and #3207 (MND.GFP.FRB.IL2RB) AAV (two-loci dual editing). Editing conditions were varied according to the table with different % of viral stock and either in the presence of the HDR enhancer or DMSO. Immediately following electroporation, the cells were placed in 2.5% FBS containing media (recovery media). After ˜16 h, the media was replaced with 20% FBS containing media and FACS analysis done on day 3 to determine editing rate. Cells recovered in 2.5% FBS containing medium were further grown in the presence of either TL-2 or Rapalog for an additional 10 days to monitor enrichment.

FIG. 64 depicts a graph showing that matched 10% volume of AAV HDR donors leads to improved dual editing. Editing was performed as outlined in FIG. 62. Graphs show percent double-negative, GFP-positive, mCherry-positive and GFP/mCherry double-positive populations within the dual-edited cells 3 days post-editing with varying amounts of #3207 and #3251 AAV in the presence of 30 uM HDR enhancer or DMSO.

FIG. 65 provides data showing robust enrichment of two-loci dual-edited CD4+ T cells with rapalog selection; with optimal results using 2.5% FBS media and matched 10% volume of AAV donor. Editing was performed as outlined in FIG. 62. Graphs show the cells from FIG. 10, (edited in 2.5% serum, matched 10% virus+/−HDR enhancer) as percent double negative, GFP positive, mCherry positive, and GFP/mCherry double positive cells within the editing population following contact with IL-2 or Rapalog for 10 days.

FIG. 66 provides a diagram of exemplary split-CISC constructs for insertion of islet-specific TCR and FOXP3 and enrichment of dual-edited cells using a two-loci dual-editing strategy. The IL-2 CISC (chemically induced signaling complex) is split onto 2 different constructs and co-expressed with either T1D4 TCR or FOXP3 (#3243 and #3252 respectively). Each construct contains half of a heterodimeric rapamycin binding complex (FKBP and FRB domains), along with the chimeric endoplasmic reticulum targeting domain fused to one half of the IL-2R signaling complex (IL-2RB or IL-2RG) trans-membrane and intracellular domains. Delivery of cDNA encoding each CISC component co-expressed with the T1D4 TCR/FOXP3 to primary human CD4+ T cells allows us to only expand cells that contain both CISC components and thus are also dual edited for T1D4 TCR and FOXP3 expression.

FIG. 67 shows an exemplary strategy for single locus dual-editing with capture of TRAC promoter. Schematic of AAV HDR-editing constructs designed for dual-editing within the TRAC locus to introduce: (top) (#3240 FOXP3 expression and split CISC and (bottom) (#3258) in-frame knock-in to TRAC exon 1 to drive expression of T1D4 TCR and split CISC using the TRAC endogenous promoter.

FIG. 68 shows that TRAC locus HDR editing disrupts TCR expression and mediates robust transgene expression via the endogenous TRAC enhancer-promoter. Panel A depicts an editing strategy for in-frame integration of a mCherry-Split-CISC cassette at the endogenous TRAC locus. By using a gRNA targeting the TRAC exon 1, in-frame integration of a marker fluorophore (mCherry) followed by the FRB-IL2RB CISC separated by a P2A element (construct #3253) allows for expression driven by the endogenous TRAC promoter, while disrupting expression of the endogenous TCR. Panel B depicts a timeline showing steps for AAV #3253 editing of CD4+ T cells. Cells were bead-stimulated (CD3/CD28) for 3 days prior to editing. Panel C depicts an analysis: seven days post-editing, cells were analyzed by flow for CD3 and mCherry expression. Flow cytometry plots show significant expression of mCherry with concomitant loss of CD3 in edited cells compared to mock-edited and AAV-only controls.

FIG. 69 shows comparison of mCherry expression mediated via the TRAC endogenous promoter vs. MND promoter. Gene editing was performed as shown in FIG. 67 using alternative HDR donors (#3253 vs. #3208) to assess the relative expression activity from the TRAC endogenous promoter vs. MND promoters, respectively. Flow cytometry plots shows that the level of mCherry expression when driven off the endogenous promoter (P2A.mCherry.FRB.IL2RB (#3253)) is lower than compared to when driven by the MND promoter (MND.mCherry.FKBP.IL2RG (#3208). The bottom row of panels shows data from a repeat experiment performed using the #3253 donor.

FIG. 70 show exemplary alternative dual-editing strategies for targeting the TRAC and/or FOXP3 loci and that utilize in-frame knock-in constructs to capture the TRAC endogenous promoter. Schematic of exemplary AAV donor constructs for testing single-locus and two-loci dual-editing strategies and to generate antigen-specific airT with IL-2 CISC selection capacity. T1D4 TCR is shown as a representative TCR that can be replaced by alternative TCRs based upon disease target and other relevant features for therapeutic application. IL-2 DISC constructs are similarly applied.

FIG. 71 relates to dual-editing of human CD4+ T Cells using decoy-CISC (split-DISC) constructs. Panel A depicts a diagram of Split IL-2 DISC HDR knock-in construct (#3280) for selection of dual-edited cells in Rapamycin. To generate the split decoy-CISC (split-DISC), the free FRB domain for cytoplasmic Rapamycin sequestration was added to the MND.mCherry.FKBP.IL2RG construct to generate (MND.mCherry.FKBP.IL2RG.FRB (#3280)). Each repair template (#3280 and #3207, not shown) is flanked by identical homology arms matched to a gRNA targeting the TRAC locus. Edited CD4+ T cells incorporating one copy of each construct are predicted to selectively expand under Rapalog or Rapamycin treatment. Panel B depicts a timeline showing steps for dual AAV editing of CD4+ T cell using AAV #3280 and #3207), expansion with Rapalog/Rapamycin and analysis of enriched cells. Cells were bead-stimulated (CD3/CD28) for 3 days prior to editing. Two days post-editing, cells were analyzed by flow for GFP and mCherry expression, and then expanded in media containing 50 ng/ml human IL-2, 100 nM Rapalog or 10 nM Rapamycin. Panel C depicts flow plots show the percentage of GFP/mCherry double-positive cells on day 3 post-editing.

FIG. 72 shows that dual editing of human CD4+ T cells with split-DISC constructs generates Rapamycin-selectable cells. Dual-editing was performed as described in FIG. 70. Panel A depicts flow plots show percent double-positive GFP/mCherry cells following 8 days in the presence of 50 ng/mL human IL-2, 100 nM Rapalog (AP21967), 10 nM Rapamycin, or no treatment. Panel B depicts histograms quantitate percent double-negative, GFP-positive, mCherry-positive and GFP/mCherry double-positive cells within the dual-edited cells following enrichment in IL-2, Rapalog (AP21967), Rapamycin or no treatment.

FIG. 73 show exemplary constructs for in vivo testing of dual-edited Tregs (split-DISC). Diagram of FOXP3 split IL-2 DISC HDR knock-in construct (#3262) to be paired with a T1D4 CISC construct (#3243) for Rapamycin selection of dual-edited cells. CISC components are split between 2 constructs and co-expressed with either HA-FoxP3 or T1D4 TCR. The FOXP3 CISC construct also contains the FRB domain and is predicted to protect mTOR signaling in the presence of Rapamycin (FOXP3 DISC construct). Each repair template is flanked by identical homology arms matched to a gRNA targeting the TRAC locus. Edited CD4+ T cells incorporating one copy of each construct may selectively expand under both Rapalog and Rapamycin treatment.

FIGS. 74-94 provide additional schematics and data related to generation and characterization of murine airT cells.

FIG. 74 depicts repair templates used in murine Foxp3 editing. Diagram of alternative AAV.GFP.KI and AAV.LNGFR.P2A constructs that were developed and tested in murine T cells for editing efficiency, FOXP3 expression and suppressive function.

FIG. 75 depicts a schematic showing methods used for generation of murine airT using the MND.GFP.KI (or alternative) HDR donor construct.

FIG. 76 relates to generation and enrichment of murine airT cells utilizing alternative promoters to express endogenous Foxp3. Flow cytometry plots showing LNGFR and GFP expression prior to and post LNGFR enrichment via FACS sorting in mock, MND.GFP.KI (#1331), MND.LNGFR.P2A (#3189 and 3261), PGK.LNGFR.P2A (#3227) and EF-1a-LNGFR.P2A (#3229) edited cells. Upper plots show initial editing rates and lower plots show enrichment post FACS sorting. Data indicate that airT can be generated with each of the candidate donor constructs.

FIG. 77 depicts expression levels of Foxp3 in murine airT using alternative homology donor cassettes. Panel A depicts flow cytometry plots showing LNGFR and GFP expression in HDR-edited splenic T cells. Panels show un-manipulated C57BL/6 control cells; mock-edited, MND.GFP.KI (#1331), MND.GFP.KI with UCOE (#3213), and PGK.GFP.KI (#3209)-edited C57 BL/6 murine T cells, respectively. Panel B depicts flow histograms showing FOXP3 expression from the data in panel A. Panel C depicts a bar chart showing FOXP3 MFI in nTreg and edTreg/airT generated with the indicated alternative HDR-donor constructs. MND promoter containing donors mediate the highest levels of FOXP3 expression.

FIG. 78 depicts design of, and results from, an in vitro suppression assay using murine tTreg or airT. A. airT cells used for in vitro suppression assay were enriched by FACS sorting at day 2 post editing and resuspended into RPMI media containing 10% FBS. nTregs (CD4+CD25+), T_{eff} (CD4+CD25−) and antigen presenting cells (CD4−CD25−) were isolated from the combined spleen and lymph node cells of 8 to 10 weeks-old C56BL/6 mice by column enrichment. Enriched 5×10⁶ T_{eff} were resuspended in 2 ml of PBS and labeled with cell trace violet (CTV) for 15 minutes at 37° C., then washed and resuspended in media before their addition in the suppression assay. To set up the assay, 1.25×10⁵ irradiated APCs (2500 rad) were co-cultured with 0.25×10⁵ T_{eff} and a titrated number of nTregs and airT in the presence of 1 mg/ml anti-CD3 in a U bottom 96 well tissue culture plate with total volume of 300 ul media and incubated at 37° C. CO₂ incubator for four days. At day 4, cells were washed twice with PBS and stained with live/dead indicator, anti-CD4, anti-CD45 and anti-CD25, and analyzed by FACS (LSRII) for the suppression of T_{eff} proliferation by airT. B) Representative flow data showing a reduction of T_{eff} proliferation in the presence of airT cells.

FIG. 79 depicts results from testing murine airT suppressive function in vitro. Flow cytometry plots showing cell trace violet labeled CD4+ T cells in the presence and absence of mock-, MND.GFP.KI- (#1331), or MND.LNGFR.P2A- (#3261-edited T cells, or nTregs from C57 BL/6 mice. These data demonstrate that murine airT (generated with the MND.GFP.KI or MND.LNGFR.P2A HDR donors) and nTregs exhibit comparable, robust in vitro suppressive function.

FIG. 80 depicts in vitro suppressive function of murine airT with alternative promoters. Flow cytometry plots showing cell trace violet-labeled CD4+ T cells in the presence and absence of mock-edited, MND.GFP.KI- (#1331), MND.LNGFR.P2A- (#3261), PGK.LNGFR.P2A- (#3227), and EF-1a.LNGFR.P2A (#3229)-edited T cells, or nTregs from C57 BL/6 mice. Murine airT with MND promoter exhibit suppressive function that is comparable to nTreg. In contrast, airT using the PGK or EF-1a promoters exhibit only limited or no suppression.

FIG. 81 depicts the design of an experiment to compare sorted vs. column-purified enriched LNGFR+ edited cells in an NSG adoptive transfer model. The table lists the number of recipient NSG host animals, and source and number of adoptively transferred control, airT or nTreg cells in each of the 5 experimental cohorts

FIG. 82 depicts flow analysis of LNGFR.P2A-edited NOD BDC2.5+ murine cells prior to and post-column purification. Panel A depicts flow cytometry plots showing LNGFR expression in mock-, and MND.LNGFR.P2A- (#3189)-edited cells. Panel B depicts flow cytometry plots showing LNGFR expression in MND.LNGFR.P2A (#3189)-edited cells post enrichment via sorting. FACS sorting consistently enriched to edTreg products of >90% purity for use in in vitro and in vivo studies.

FIG. 83 depicts a flow analysis of edited murine cells before and after column enrichment. Flow cytometry plots showing MND-LNGFR.P2A (#3189) edited cells prior to- and post-column enrichment. In this example of enrichment, 72×10⁶ cells with initial editing rate of ˜7% were added to an anti-LNGFR column yielding 2×10⁶ edTreg with >84% purity.

FIG. 84 depicts an experimental design and results assessing islet antigen-specific airT function in the NSG adoptive transfer model: comparison of FACS-sorted and column-enriched airT. Islet antigen-specific (BDC) airT (generated using the HDR donors, 3261 or 3389), or antigen-specific nTregs were adoptively transferred by retro-orbital (R.O.) delivery into adult, 8-10 wk old recipient NSG mice, followed by infusion of antigen-specific T_{eff} cells. Mice were monitored for development of diabetes for up to 60 days. Graph shows the percent of diabetic mice after receiving effector cells plus the designated mock-edited, MND.LNGFR.P2A-edited (FACS sorted or column enriched), or nTreg cells from NOD BDC2.5 mice. Column-enriched Ag-specific MND.LNGFR.P2A airT reduced diabetes incidence in NSG mice and shows comparable function to FACS-sorted airT. Higher doses of column-enriched MND.LNGFR.P2A airT or nTreg fully protected recipient animals from development of diabetes.

FIG. 85 depicts a comparison of in vivo function of airT generated using alternative promoters in the NSG adoptive transfer model. Engineered antigen-specific (BDC) airT (generated using either the MND or PGK promoter; donor constructs 1331 or 3209, respectively), or antigen-specific nTregs were adoptively transferred into NSG recipient mice followed by infusion of antigen-specific T_{eff} cells. Mice were monitored for development of diabetes for up to 60 days. Graph shows the percent of diabetic mice after receiving effector cells (5×10⁴) plus the designated mock edited, MND.GFP.KI (#1331), PGK.GFP.KI (#3209) airT or nTreg cells (5×10⁴) from NOD BDC2.5 mice. Antigen-specific airT with the MND promoter prevented diabetes development in all recipient mice. nTreg prevented disease in ⅘ recipient mice. In contrast, antigen-specific airT that incorporated the PGK promoter had little or no protective effect. These data directly demonstrate that protection from T1D is specific to airT generated using the MND promoter to drive Foxp3 expression supporting the use of this architecture in human trials for T1D or other immune diseases.

FIG. 86 shows that islet Ag-specific MND.GFP.KI airT persist in vivo in the target organ (pancreas) for at least 60 days and exhibit a stable phenotype. Flow cytometry plots showing FOXP3 and GFP expression in MND.GFP.KI (#1331) NOD BDC2.5 airT recovered in the pancreas at day 60 following adoptive transfer in NSG mice. Data shows results from two mice. Recipient mice also exhibit expansion of endogenous Treg or iTreg (FOXP3+. GFP− CD4 T cells) derived from the input T_{eff} population (likely secondary to additional beneficial bystander impacts of airT delivery).

FIG. 87 depicts design and results of CRISPR-based targeting of the murine Rosa26 Locus for Knock-in/knock-out. Panel A depicts the Rosa26 locus was selected as a model of a safe-harbor HDR integration site for murine T cells. Position of the two novel gRNAs (gRNA_1 and gRNA_2) within the murine Rosa26 locus. gRNAs from Pesch et. al. and Wu et. al. comprise previously published gRNAs within this locus region. Panel B depicts on-target site-specific activity as measured by ICE (Inference of CRISPR Edits) demonstrates specific indel induction using R26 gRNA_1 in Rosa26 after Cas9-RNP delivery to primary mouse CD4+ T-Cells.

FIG. 88 depicts an experimental outline for HDR editing at the mouse Rosa26 locus. Panel A depicts a diagram of AAV construct #3245 used in this experiment. After HDR-based editing in mouse T cells, the MND promoter drives expression of GFP. Panel B depicts a timeline of experimental procedures. Murine C57BL/6J CD4+ T cells were isolated and bead stimulated (CD3/CD28) for 3 days prior to editing. Cells were evaluated for GFP by flow cytometry at days 3 and 8 post editing.

FIG. 89 depicts data demonstrating HDR-based editing within the Rosa26 locus in murine CD4+ T cells. CD4+ T cells were edited as outlined in FIG. 88 and assessed at Day 3 by flow cytometry. Panel A depicts flow plots showing GFP expression in mock-edited, AAV #3245 alone and AAV #3245/RNP-edited cells at 3 days post editing. Panel B depicts histograms show percent viability, % GFP positive cells and high GFP+ cells within the edited population.

FIG. 90 shows that murine T cells maintain stable expression of GFP following HDR editing of the Rosa26 locus. CD4+ T cells were edited as outlined in FIG. 88 and assessed at Day 8 by flow cytometry. Panel A depicts flow plots show GFP expression in mock edited, AAV #3245 alone and AAV #3245/RNP edited cells 8 days post editing. Panel B depicts histogram shows % GFP positive cells within the edited population.

FIG. 91 depicts schematics of AAV HDR donor constructs for expression of murine Foxp3- and P2A- linked LNGFR within the Rosa26 locus in murine T cells. Repair templates are flanked by 300 bp homology arms matched to R26_gRNA_1 cleavage site and contain alternative promoters (MND or PGK) driving expression of mFOXP3 and LNGFR. In addition, a cassette containing a Foxp3 4× CDK phosphorylation site mutant is included as this construct is predicted increase the stability of Foxp3.

FIG. 92 relates to lentiviral CISC constructs used to transduce murine CD4+ T cells and test selective expansion with Rapalog. Panel A depicts a diagram of lentiviral construct #1272. This construct was developed to assess proof-of-concept for enrichment of murine T cells using human CISC components in the presence of Rapalog. After transduction of mouse T cells, the MND promoter drives expression of mCherry linked to IL-2 CISC components (FKBP-IL2RG and FRB-IL2RB). Panel B depicts a timeline of experimental procedures. Murine C57BL/6J CD4+ T cells were bead stimulated (CD3/CD28) for 3 days prior to transduction. Cells were evaluated for mCherry by flow cytometry at days 2 and 5 post-transduction.

FIG. 93 shows that murine CD4+ T cells transduced with lentiviral CISC show robust enrichment in Raplog. Panel A depicts flow plots show mCherry expression 2 days following mock or lentiviral transduction (#1272) of murine CD4+ T cells. Panel B depicts flow plots show mCherry expression in mock murine cells treated with IL-2, IL-7 and IL-15, or lentiviral (#1272) transduced murine cells that are treated with either IL-2, IL-7 and IL-15, Rapalog alone, or Rapalog+bead stim.

FIG. 94 shows that airT cells suppress proliferation of CD8+ T cells, as well as CD4+ T cells.

FIG. 95 depicts a schematic of a process for generating antigen-specific airT cells by stimulation with a model antigen peptide (MP) and editing for FoxP3 expression.

FIG. 96 depicts antigen-specific suppression by MP peptide-specific airT cells. Briefly: T_{eff}: day 23 T cells stimulated by MP peptide (right) or HA peptide (left); Treg: day 23 edited cells specific for MP peptide (right) or HA peptide (left), edited by CRISPR/Cas9 and AAV Foxp3-MND-LNGFRki; APC: irradiated autologous CD4−CD25+ cells DMSO or HA peptide 5 μg/ml; 6 day incubation.

FIG. 97 shows that airT cells show suppressive activity on T_{eff} proliferation. Briefly: 3 day incubation; for bead suppression: T_{eff}+Treg (untd edTreg, T1D5-1 airT, or T1D5-1 mock); For Ag-specific suppression: T1D5-1 T_{eff}+Treg; T_{eff} gate: CD4+ CD11c-CTV+ EF670-mTCRb+ gate.

FIG. 98 depicts suppression of cytokine production in T_{eff} by airT. Briefly: T1D4 T_{eff}; Treg (d10): T1D4 mock or T1D4 airT; and Peptide 1 μg/ml; for a 3 day incubation.

FIG. 99 depicts antigen-specific and bystander suppression on T_{eff} by airT. Briefly: T_{eff} 1.25×10⁴; Treg 2.5×10⁴; APC 1×10⁵; and Peptide 5 μg/ml.

FIG. 100 depicts antigen-specific and bystander suppression on T_{eff} by airT. Briefly: T_{eff} 1.25×10⁴; Treg 2.5×10⁴; APC 1×10⁵; and Peptide 5 μg/ml.

FIG. 101 shows bystander suppression of T_{eff} cytokine production. Briefly: T1D5-2 T_{eff}; Treg (d10): T1D4 mock or T1D4 edTreg; and Peptide 1 μg/ml; and 3 day incubation.

FIG. 102 shows dose response of TCR: proliferation assay. Briefly: mTCR expression data: day 8 post-transduction; Proliferation assay: day 11 cells, and 4 day incubation.

FIG. 103 shows validation of islet Ag-specific TCR expression: mTCRb expression & proliferation assay. Briefly: T cells: day 9 post transduction, labeled with Cell Trace Violet; APC: irradiated CD4−CD25+ cells; and 5 day incubation.

FIG. 104 shows that antigen-specific GFP+ airT can be detected in the pancreas. See also FIG. 107 and FIG. 116.

FIG. 105 relates to generation and enrichment of murine LNGFR+ airT cells for in-vivo suppression studies. See also FIG. 114.

FIG. 106 shows that Ag-specific MND.LNGFR.P2A-airT completely prevented diabetes in NSG mice. See also FIGS. 115, 134 and 135.

FIG. 107 shows that antigen-specific GFP+ airT can be detected in the pancreas.

FIG. 108 shows schematics and data related to an exemplary IL-2 CISC of the present disclosure.

FIG. 109 shows that in vivo rapamycin contact promotes CISC cell persistence.

FIG. 110 shows a schematic of an exemplary edited cell of the present disclosure.

FIG. 111 relates to gRNA selection for TRAC locus targeting.

FIG. 112 relates to a dual editing strategy with IL-2 Split-CISC components targeted to the TRAC locus.

FIG. 113 shows that CISC-engagement selects for dual-edited cells in-vitro.

FIG. 114 depicts a flow analysis of LNGFR.P2A-edited NOD BDC2.5+ murine cells prior to and post column purification. Panel A depicts a flow cytometry plots showing LNGFR expression specifically in MND.LNGFR.P2A (#3261)-edited cells but not in mock cells. Panel B depicts a flow cytometry plots showing LNGFR expression in MND.LNGFR.P2A (#3261)-edited cells in the flow through (F.T.) and eluted sample post enrichment via column purification. Column enrichment led to an airT product of 74.5% purity for use in in vivo studies.

FIG. 115 depicts an assessment of islet antigen-specific airT function in the NSG adoptive transfer model: Islet antigen-specific (BDC) airT (generated using the HDR donor 3261), or antigen-specific nTregs (50K), were adoptively transferred by retro-orbital (R.O.) delivery into adult, 8-10 wk old recipient NSG mice followed by infusion of 50K antigen-specific T_{eff} cells. Panel A depicts a flow cytometry plots showing the CD4 and CD25 profile of nTreg and LNGFR+ expression in MND.LNGFR.P2A (#3261)-edited cells. Panel B depicts a graph: mice were monitored for development of diabetes for up to 49 days. Graph shows the percent of diabetic mice after receiving effector cells plus the designated mock-edited, MND.LNGFR.P2A-edited (column enriched), or nTreg cells from NOD BDC2.5 mice. Column-enriched Ag-specific MND.LNGFR.P2A-airT completely prevented diabetes in NSG mice.

FIG. 116 shows that islet Ag-specific MND.GFP.KI airT persist in vivo in the target organ (pancreas) for at least 49 days and exhibit a stable phenotype. Flow cytometry plots showing LNGFR and FOXP3 expression in NOD BDC2.5 airT recovered in the pancreas at day 49 following adoptive transfer in NSG mice.

FIG. 117A depicts flow plots of mTCRb expression gated on CD4+ cells day 9 post-transduction.

FIG. 117B depicts flow plots of CD4+ T cells transduced with RA Ag-specific TCRs labeled with CTV and co-cultured with APC (irradiated PBMC) and their cognate peptide or DMSO for 3 days.

FIG. 118B depicts a polyclonal suppression assay and an antigen-specific suppression assay using enolase-specific edTreg.

FIG. 118C depicts a graph of percentage suppression of Teff proliferation by no Treg, untd edTreg, Enol edTreg, or mock in the presence of a-CD3/CD28 (black) or APC and enolase peptide (grey) calculated from percentage proliferation in FIG. 118B.

FIG. 119A depicts flow plots of mTCRb expression in untransduced edTreg and CILP297-1 edTreg gated on LNGFR+ Foxp3+ in edited cells transduced with no LV and LV CILP297-1-TCR, respectively.

FIG. 119B depicts a polyclonal suppression assay and an antigen-specific suppression assay using CILP-specific edTreg.

FIG. 119C depicts a graph of percentage suppression of CILP Teff proliferation by no Treg, untd edTreg, CILP edTreg, or mock in the presence of a-CD3/CD28 (black) or APC and CILP peptide (grey) calculated from percentage proliferation in FIG. 119B.

FIG. 120A depicts flow plots of mTCRb expression in untransduced edTreg and Vim418 edTreg gated on LNGFR+ Foxp3+ in edited cells transduced with no LV and LV Vim418-TCR, respectively.

FIG. 120B depicts a polyclonal suppression assay and an antigen-specific suppression assay using vimentin-specific edTreg.

FIG. 120C depicts a graph of percentage suppression of Vim Teff proliferation by no Treg, untd edTreg, Vim edTreg, or mock in the presence of a-CD3/CD28 (black) or APC and Vimentin peptide (grey) calculated from percentage proliferation in FIG. 120B.

FIG. 121A depicts flow plots show mTCRb expression in untransduced, Agg520, and Vim418 edTreg gated on LNGFR+ Foxp3+ in edited cells transduced with no LV, LV Agg520-TCR, and LV Vim418-TCR, respectively.

FIG. 121B depicts a polyclonal suppression assay using Agg520 Teff and edTreg or mock specific to Agg520 or Vim418.

FIG. 121C depicts a graph of percentage suppression of Agg520 Teff proliferation by no Treg, untd edTreg, Agg edTreg/mock, or Vim edTreg/mock calculated from percentage proliferation in FIG. 121B.

FIG. 121D depicts an antigen-specific and a bystander suppression assay using Agg520 Teff and edTreg or mock specific to Agg520 or Vim418.

FIG. 121E depicts a graph of percentage suppression of Agg520 Teff proliferation by no Treg, edTreg or mock calculated from percentage proliferation in FIG. 121D.

FIG. 122A depicts flow plots of mTCRb expression in untransduced, CILP297-1, and Vim418 edTreg gated on LNGFR+ Foxp3+ in edited cells transduced with no LV, LV CILP297-1-TCR, and LV Vim418-TCR, respectively.

FIG. 122B depicts a polyclonal suppression assay using CILP297-1 Teff and edTreg or mock specific to CILP297 or Vim418.

FIG. 122C depicts a graph of percentage suppression of CILP Teff proliferation by no Treg, untd edTreg, CILP edTreg or mock, or Vim edTreg or mock calculated from percentage proliferation in FIG. 122B.

FIG. 122D depicts an antigen-specific and bystander suppression assay using CILP297-1 Teff and edTreg specific to CILP297 and Vim418.

FIG. 122E depicts a graph of percentage suppression of CILP Teff proliferation by no Treg, edTreg or mock calculated from percentage proliferation in FIG. 122D.

FIG. 123A depicts flow plots of mTCRb expression and LNGFR/Foxp3 expression in edited cells expressing SLE3-TCR on day 7.

FIG. 123B depicts a polyclonal suppression assay and an antigen-specific suppressing assay using SLE-specific edTreg.

FIG. 124A depicts a schematic diagram of AAV HDR-donor constructs designed to introduce split-CISC elements into the TRAC locus using a single locus dual editing approach.

FIG. 124B depicts a timeline for key steps for dual AAV editing of CD4+ T cells and expansion with Rapalog.

FIG. 125A depicts flow plots of T1D4 and FOXP3 expression in mock edited, single edited and dual-edited cells (using 10% volume of both #3243 and #3240 AAV) at Day 3 post editing.

FIG. 125B depicts flow plots of T1D4 and CD4 expression in mock edited, and mixed edited cells.

FIG. 125C depicts histograms of percent double negative, FOXP3−HA positive, T1D4 positive and FOXP3/T1D4 double positive cells within the dual edited cells.

FIG. 125D depicts histograms of percent CD3 knockout in FOXP3/T1D4 dual edited cells vs. mock edited cells.

FIG. 126A depicts flow plots of viability and T1D4 and FOXP3 expression in dual-edited cells treated with either 50 ng/mL IL-2 (upper panels) or 100 nM Rapalog (AP21967; lower panels) for 7 days.

FIG. 126B depicts flow plots of CTLA4 expression of T1D4/FOXP3 double positive vs. double negative cell populations treated with either 50 ng/mL IL-2 (upper panels) or 100 nM Rapalog (AP21967; lower panels) for 7 days.

FIG. 127A depicts flow plots of viability (right plots) and T1D4 and FOXP3 expression (left plots) in dual-edited cells following treatment with 50 ng/mL IL-2 (upper plots) vs. 100 nM AP21967 (lower plots) after recovery in IL-2 medium.

FIG. 127B depicts a graph of fold enrichment of double positive T1D4/FOXP3 cells treated with either 50 ng/mL IL-2 or 100 nM Rapalog (AP21967) over a 10 day period with the last 3 days being in recovery media containing IL-2.

FIG. 128A depicts a diagram of Split IL-2 DISC HDR knock-in construct (#3280), for selection of dual-edited cells in either Rapamycin or Rapalog.

FIG. 128B depicts a timeline of key steps for dual AAV editing of CD4+ T cell using AAV #3280 and #3207, expansion with Rapalog/Rapamycin and analysis of enriched cells.

FIG. 129A depicts flow plot of mCherry and GFP expression in dual edited cells (10% culture volume of #3280 and #3207 AAV donors, respectively) four days post editing. Viral titers were 3.30E+12 and 3.1E+10 for #3280 and #3207 respectively. Dual-edit 4 million total cells initial dual positive rate: 4.47%. gRex vessel was seeded with 7.6 million total cells, 340,000 double-positive.

FIG. 129B depicts flow plots of viability (upper panel) and GFP and mCherry expression (lower panel) following the seeding of 7.6 million edited cells in gREX and 7 day expansion in the presence of AP21967 leading to 32-fold expansion of double-positive cells. Total double positive cells in gRex: 11.1 million.

FIG. 130A depicts a timeline of steps for dual AAV editing of CD4+ T cell using AAV #3280 and #3207, expansion with Rapalog and analysis of enriched cells.

FIG. 130B depicts flow plots of mCherry and GFP expression in dual edited cells (10% #3280 and 10% #3207 AAV). Viral titers were 3.30E+12 and 3.1E+10 for #3280 MND.mCherry.FKBP.IL2RG.FRB and #3207 pAAV.MND.GFP.FRB.IL2RB respectively. Edit 10 million total cells, initial dual positive rate: 2.37%. Seeded gRex with 9.1 million total cells 216,000 double-positive.

FIG. 131 depicts flow plots of viability and GFP and mCherry expression following the seeding of edited cells in gREX and 7 day expansion in the presence of AP21967. The results after 7 day expansion included Total double positive cells in gRex: 9.7 million; about 45-fold expansion from original 216,000 double positive cells.

FIG. 132A depicts a design for in vitro suppression assay using mouse edTreg or nTreg.

FIG. 132B depicts representative flow date showing a reduction of BDC2.5+ Teff proliferation in the presence of BDC2.5+ edTreg cells.

FIG. 133 depicts flow cytometry plots showing cell trace violet labeled CD4+ T cells in the presence and absence of mock, MND.LNGFR.p2A (#3261) edited Treg or nTregs from NOD BDC2.5+ mice. Murine Islet TCR+ edT_{reg} (generated with the MND.LNGFR p2A (#3261) HDR donors) and tTregs exhibit antigen-specific in vitro suppressive function. 50 K Teff+anti-CD3 (1 μg/ml)+200 K irradiated APCs (2500 rad). Analysis @Day 4. CTV=cell trace violet. Data shown: 1:1 (Teff to Treg ratio).

FIG. 134 depicts a graph of the percent of diabetic mice after receiving effector cells plus the designated mock edited, MND.LNGFR.P2A edited or nTreg cells from NOD BDC2.5 mice. Similar to nTregs, MND.LNGFR p2A edTregs completely prevented the onset of diabetes while mock edited control cells did not show any impact on disease onset.

FIG. 135 depicts a graph of the percent of diabetic mice after receiving effector cells plus the designated mock edited, MND.LNGFR.P2A edited or nTreg cells from NOD BDC2.5 mice in a repeat experiment. Column enriched Ag-specific LNGFR p2A edTregs completely prevented (Day 33) diabetes in NSG mice

FIG. 136A, FIGS. 136B and 136C each show a TABLE listing amino acid sequences of TCR alpha and beta CDR3 and J regions of TCR that specifically recognize antigens associated with pathogenesis of autoimmune, allergic, or inflammatory conditions.

FIG. 137 shows a TABLE listing amino acid sequences of TCR alpha and beta CDR3 and J regions of TCR that specifically recognize antigens associated with pathogenesis of autoimmune, allergic, or inflammatory conditions.

FIG. 138 shows a TABLE listing amino acid sequences of J regions of TCR that specifically recognize antigens associated with pathogenesis of autoimmune, allergic, or inflammatory conditions.

FIG. 139A shows a TABLE listing nucleotide sequences encoding TCR alpha and beta chain V regions of TCR that specifically recognize antigens associated with pathogenesis of autoimmune, allergic, or inflammatory conditions.

FIG. 139B shows a TABLE listing amino acid sequences of J regions of TCR that specifically recognize antigens associated with pathogenesis of autoimmune, allergic, or inflammatory conditions.

FIG. 140A shows a TABLE listing nucleotide sequences encoding TCR alpha and beta chain V regions of TCR that specifically recognize antigens associated with pathogenesis of autoimmune, allergic, or inflammatory conditions.

FIG. 140B shows a TABLE listing amino acid sequences of TCR alpha and beta J regions of TCR that specifically recognize antigens associated with pathogenesis of autoimmune, allergic, or inflammatory conditions.

FIG. 141 shows a TABLE listing amino acid sequences of antigenic epitopes recognized by specific TCR for a CYP2D6 antigen associated with autoimmune hepatitis type 2.

FIG. 142 shows a TABLE listing amino acid sequences of antigenic epitopes recognized by specific TCR for a BP230 or a BP180 antigen associated with bullous pemphigoid.

FIG. 143A shows a TABLE listing amino acid sequences of polypeptide antigens associated with pathogenesis of autoimmune, allergic, and/or inflammatory conditions, containing antigenic epitopes recognized by specific TCR and amino acid sequences of TCR alpha and beta CDR3 regions of TCR that specifically recognize the antigens.

FIG. 143B shows a TABLE listing amino acid sequences of polypeptide antigens associated with pathogenesis of autoimmune, allergic, and/or inflammatory conditions, containing antigenic epitopes recognized by specific TCR and amino acid sequences of TCR alpha and beta CDR3 regions of TCR that specifically recognize the antigens.

FIG. 144 shows a TABLE listing amino acid sequences of polypeptide antigens associated with pathogenesis of autoimmune, allergic, or inflammatory conditions, containing antigenic epitopes recognized by specific TCR and amino acid sequences of TCR alpha and beta CDR3 regions of TCR that specifically recognize the antigens.

FIG. 145 shows a TABLE listing certain nucleic acid sequences useful with embodiments provided herein including guide RNA (gRNA), and an AAV vector containing FOXP3 editing sequences.

FIG. 146 depicts schematic maps including: (panel A) a FOXP3 knock-in construct (3324) comprising elements encoding an FKBP-IL2RG polypeptide and an FRB polypeptide; (panel B) a TRAC-targeting HDR construct (3243) comprising elements encoding a T1D4 polypeptide and an FRB-IL2B polypeptide; (panel C) a TRAC-targeting HDR construct (3333) comprising elements encoding a T1D4 polypeptide and an FRB-IL2Bmin polypeptide, which comprises a truncated intracellular IL-2 receptor beta signaling domain.

FIG. 147 depicts FACS analysis of CD4+ T cells transduced with either constructs 3333/3324 (row A), 3243/3324 (row B), or control (row C).

FIG. 148 depicts (panel A) a FACS analysis of CD4+ T cells transduced with either constructs 3333/3324 (upper row), 3243/3324 (lower row) and treated for 7 days with 100 nM AP21967 (Rapalog), and includes viability versus cell size (FSC-A), T1D4 expression versus CD3 expression, and T1D4 expression versus HA-tagged FOXP31; and (panel B) a graph of double positive fold enrichment in the CD4+ T cells.

FIG. 149 depicts schematic maps of alternative TRAC-targeting HDR constructs including: (upper) a construct in which a micro-CISC component is proximal to a MND promoter; and (lower) a construct in which a TCR (T1D4) component is proximal to a MND promoter.

FIG. 150A depict (panel A) a graph of double positive fold enrichment in CD4+ T cells transduced with 3324/3323 constructs, and treatment over 7 days in either 50 ng/mL IL-2, 100 nM AP21967, or 10 nM rapamycin; and (panel B) a graph of double positive fold enrichment in CD4+ T cells transduced with 3324/3333 constructs, and treatment over 7 days in either 50 ng/mL IL-2, 100 nM AP21967, or 10 nM rapamycin.

FIG. 150B depicts schematic maps of contracts comprising sequences encoding a truncated FRB-IL2RB proximal to an MND promoter, (3323 and 3354), and a construct in which sequences encoding the truncated FRB-IL2RB were distal to an MND promoter with intervening sequences encoding a T1D4 TCR polypeptide (3234).

FIG. 150C depicts a FACS analysis of cells transduced with constructs shown in FIG. 150B including a combination of a FOX3P targeting construct (3324) and either the 3323, 3354 or 3243 construct. The enrichment capacity for the alternative TRAC targeting constructs was compared in an 8-day time course using 100 nM AP21967 (Rapalog), or 10 nM Rapamycin.

FIG. 150D depicts graphs of absolute vs fold enrichment between the different dual editing groups of FIG. 150B and FIG. C.

FIG. 151 depicts (panel A) a schematic map of a CISC construct with either a MND promoter (1272) or an EF1a promoter (3312); and (panel B) a FACS analysis of CD4+ T cells transduced with either the 1272 or the 3312 construct for mCherry signaling versus cell size.

FIG. 152 depicts (panel A) a FACS analysis of CD4+ T cells transduced with either the 1272 or the 3312 construct, following 7 days in 100 nM AP21967 (Rapalog) for viability versus cell size (FSC-A), and mCherry versus cell size; and (panel B) a graph of double positive fold enrichment of mCherry over 7 days in CD4+ T cells transduced with either the 1272 or the 3312 construct, and treated with 100 nM AP21967 (Rapalog).

FIG. 153 depicts (panel A) a timeline and steps of peptide stimulation to expand islet-specific T cells; (panel B) a representative FACS analysis for tetramer positive T cells specific for individual antigenic peptides; and (panel C) bar histograms of average percent tetramer positive population in CD4+ T cells measured after 12-14 days of in vitro peptide stimulation in which each bar indicates the percentage of CD4+ T cells specific for each islet antigenic peptide and in which each dot represents a different experiment.

FIG. 154 depicts (panel A) a timeline and steps for production of islet-specific edTregs, polyclonal islet-specific Teff, and monocyte-derived DC (mDC) from PBMC and the in vitro suppression assay; (panel B) histograms showing proliferation of polyclonal islet Teff in antigen-specific suppression assay; and (panel C) bar histograms showing percent suppression on proliferation of polyclonal islet Teff by polyclonal edTreg, T1D2 mock, T1D2 edTreg, 4.13 mock, or 4.13 edTreg in the presence of mDC and a pool of 9 islet-specific peptides in which percent suppression was calculated by (% proliferation with no Treg−% proliferation with edTreg)/% proliferation with no Treg×100. **P<0.001, *P<0.05, as determined by paired t-test.

FIG. 155 depicts (panel A) a graph of proliferation of T cells expressing T1D2, T1D5-1, or T1D5-2 TCR; (panel B) a TABLE listing peptide specificity and avidity of T1D2, T1D5-1, and T1D5-2 TCR; (panel C) histograms of proliferation of T1D5-2 Teff in an antigen-specific suppression assay using T1D2 edTreg or T1D5-1 edTreg; (panel D) bar histograms of percent suppression of T1D5-2 Teff proliferation by T1D2 edTreg or T1D5-1 edTreg in the antigen-specific suppression assay; (panel E) histograms of proliferation of polyclonal islet Teff in antigen-specific suppression assay; and (panel F) bar histograms of percent suppression on proliferation of polyclonal islet Teff by T1D2 edTreg or T1D5-1 edTreg in the presence of mDC and a pool of 9 islet-specific peptides.

FIG. 156A depicts (panel A) a graph of proliferation of T cells expressing islet-specific TCRs; (panel B) a TABLE summarizing peptide specificity and avidity of islet-specific TCRs; (panel C) histograms (left) of proliferation of polyclonal islet Teff in an antigen-specific suppression assay using T1D2 edTreg or 4.13 edTreg, and bar histograms (right) of percent suppression on proliferation of polyclonal islet Teff by T1D2 edTreg or 4.13 edTreg in the presence of mDC and a pool of 9 islet-specific peptides from three independent experiments using cells generated from three different T1D donors in which statistical significance was determined by paired t-test; (panel D) histograms (left) of proliferation of polyclonal islet Teff in antigen-specific suppression assay using T1D2 edTreg, GAD113 edTreg, or PPI edTregs, and bar histograms (right) of percent suppression on proliferation of polyclonal islet Teff by T1D2 edTreg, GAD113 edTreg, or PPI edTregs; (panel E) histograms (left) of proliferation of polyclonal islet Teff in antigen-specific suppression assay using T1D2 edTreg, T1D4 edTreg, or T1D5-2 edTreg, and bar histograms (right) of percent suppression on proliferation of polyclonal islet Teff by T1D2 edTreg, T1D4 edTreg, or T1D5-2 edTreg.

FIG. 156B depicts (panel A) a graph of proliferation of T cells expressing islet-specific TCRs; and (panel B) a TABLE summarizing peptide specificity and avidity of islet-specific TCRs.

FIG. 157 depicts (panel A) a schematic for generation of edTreg cells expressing islet-TCRs; and (panel B) a summary of methods used to assess antigen-specific suppression assays.

FIG. 158 depicts histograms of FACS analysis of CD4+ T cells transduced with T1D5-1-TCR (T1D5-1 Teff), labeled with CTV and co-cultured with or without EF670-labeled untransduced edTreg (untd edTreg), edTreg expressing T1D5-1 TCR (T1D5-1 edTreg), or T1D5-1 mock cells in the presence of CD3/CD28 activator beads. Upper row histograms show Teff proliferation in response to CD3/CD28 bead activation; and lower row histograms panels show antigen-specific Teff proliferation.

FIG. 159 depicts (left panel) histograms of Teff proliferation gated on CD3+CD4+ CTV+ EF670− LNGFR−; and (right panel) a graph of percent proliferation of T1D4 Teff cells with no edTreg, T1D4 edTreg, or T1D5-1 edTreg in the presence of APC and IGRP 241+305 peptides.

FIG. 160 depicts (left panel) depicts a FACS analysis, and (right panel) graphs with regard to TNF, IL-2, or IFN-g production and CD25 expression from T1D4 Teff cells gated on CD4+ CTV+ EF670−.

FIG. 161 depicts (upper panel) FACS analysis, and (lower panel) with regard to TNF, IL-2, or IFN-g production and CD25 expression from bystander T1D5-2 Teff cells gated on CD4+ CTV+ EF670−.

FIG. 162 depicts (panel A) histograms of Teff proliferation in TV-labeled T1D5-2 Teff or T1D4 Teff co-cultured with or without EF670-labeled T1D5-1 edTreg or T1D5-2 edTreg in the presence of APC and IGRP 305, IGRP 241, or IGRP 241+305 peptides; panel (B) graphs of percent proliferation of T1D5-1 Teff (top) and T1D4 Teff (bottom); and (panel C) graphs of percent TNF and IL-2 production by T1D5-2 Teff (left) or T1D4 Teff (right).

FIG. 163 depicts (panel A) histograms of Teff proliferation in response to CD3/CD28 bead activation; and (panel B) histograms show islet-specific T cell proliferation after being co-cultured for four days with no Treg, untd edTreg, T1D2 edTreg, or T1D2 mock cells in the presence of monocyte-derived DC and a pool of 9 islet-specific peptides or DMSO.

FIG. 164A depicts schematic maps including: (panel A) a full CISC TRAC hijack construct (3354) comprising elements encoding an FRB-IL2RB AA237-551 polypeptide, a full-length TCRb polypeptide, and a TRAV/TRAJ polypeptide; (panel B) a CISC component swap TRAC hijack construct (3363) comprising elements encoding an FKBP-IL2RG polypeptide, a full-length TCRb polypeptide, and a TRAV/TRAJ polypeptide; (panel C) a CISC component swap FOXP3 construct (3362) comprising elements encoding an FRB-IL2RB AA237-551 polypeptide and an FRB polypeptide. FIG. 164B depicts the efficiency of editing T cells using constructs 3363 and 3362 to disrupt endogenous TCR expression, introduce an exogenous TCRb polypeptide and TRAV/TRAJ polypeptide, promote stable FOXP3 expression, and express an FKBP-IL2RG polypeptide, FRB-IL2RB polypeptide, and FRB polypeptide. FIG. 164C depicts the frequency of double-positive cells, expressing FOXP3 and exogenous TCRb, over time after incubation with rapamycin.

FIG. 165 depicts schematic maps including: (panel A) a T1D4 full CDS CISC order swap construct (3364) comprising elements encoding an FKBP-IL2RG polypeptide, and a T1D4 polypeptide; (panel B) a A2-CAR CISC construct comprising elements encoding an FRB-IL2RB AA237-551 polypeptide, and an A2-CAR polypeptide.

FIG. 166A illustrates the process of deriving and delivering autologous ex vivo-expanded natural Treg cells or allogeneic umbilical cord blood (UCB)-derived Treg cells to a subject as a Treg therapy. FIG. 166B illustrates HDR-mediated introduction of a FOXP3 expression cassette driven by an MND promoter at the FOXP3 locus.

FIG. 167 depicts optimization of the expansion of UCB-derived EngTregs generated by HDR-dependent gene editing. Panel A depicts the experimental timeline illustrating the editing of CD4+ cells and the expansion and validation of the resulting EngTregs expressing FOXP3cDNA and LNGFR marker. Panel B depicts the expansion of UCB-derived (n=6) and PBMC-derived (n=5) CD4+ T cells between DO and D4 as shown in A. Cells are stimulated by CD3/CD28 Dynabeads for 3 days and then cultured without them for an additional 16 hours. Significance was determined through an unpaired T test. Panel C depicts the editing efficiency between UCB-derived (n=6) and PBMC-derived (n=5) cells. After RNP delivery using a Lonza 4D nucleofector, 20% v/v AAV was added to cell culture. At 2 days after editing, HDR efficiency was assessed through flow cytometry. Edited cells are represented by the percentage of live, singlet CD4 cells stained positive for LNGFR. Significance was determined through an unpaired T test. Panel D depicts the expansion of the EngTregs cultured in G-rex from UCB and PB over a 7-day period after enrichment for LNGFR.FIG. Panel E depicts the effects of IL-2 concentration and culture device on fold expansion of UCB-derived EngTregs over a 7-day period after enrichment for LNGFR. Panel F depicts the viability of UCB-derived EngTregs cultured in different devices.

FIG. 168 shows data indicating that UCB-derived EngTregs exhibit Treg phenotypes and reduction in inflammatory cytokine production. Panel A shows representative histograms depicting selected markers expressed in UCB-derived EngTregs after a 7-day expansion. Panel B shows the cytokine production relative to mock-edited cells from UCB-derived LNGFR+ EngTregs (n=5). After resting, cells were stimulated with PMA and Ionomycin in the presence of Golgi-stop for 5 hours before analyzing intracellular cytokine production by flow cytometry.

FIG. 169 shows that UCB-derived EngTregs suppress allogeneic effect T cells and provide protection against graft-vs-host disease (GvHD). Panel A shows the timeline and study design of in vivo study comparing suppression of allogeneic Teff by PBMC-derived and UCB-derived EngTregs in NSG mice. Panel B shows a survival curve over the duration of the in vivo study. 20% body weight loss is set as the humane study endpoint. Panel C depicts changes in body weight for each mouse between DO and study endpoint.

FIG. 170A shows the mechanism by which CISC activity is regulated by the presence of rapamycin. FIG. 170B illustrates an AAV donor template for CISC that is introduced upstream of the endogenous FOXP3 gene. FIG. 170C shows relative MFI of indicated Treg markers in UCB-derived EngTregs, compared to the baseline of mock-edited UCB-derived CD4+ cells. FIG. 170D shows cytokine production relative to mock-edited cells from UCB-derived CISC-expressing EngTregs expanded in tissue culture plates. Cells were treated with PMA, ionomycin, and Golgi-stop for 5 hours followed by intracellular staining for indicated cytokines. FIG. 170E shows a flow cytometry plot indicating that UCB-derived CISC-expressing EngTregs express FOXP3 and P2A, indicating production of the CISC components separated by P2A self-cleavage motifs in initial translation. FIG. 170F shows the percentage of UCB-derived CD4+ cells expressing the CISC at day 3, indicating editing efficiency, and at day 17, indicating expansion by rapamycin exposure. FIG. 170G shows the fold expansion by rapamycin treatment of mock-edited UCB-derived EngTregs, or EngTregs expressing a CISC.

FIG. 171 depicts a timeline and procedure for scale up production of UCB EngTregs.

FIG. 172A depicts immunophenotypes of LNGFR+ EngTregs from UCB and includes representative histograms depicting selected markers expressed in 2 donors of UCB-derived EngTregs after a 7-day expansion. FIG. 172B depicts bar graphs comparing the cytokine production relative to mock-edited cells from 2 donors of UCB-derived LNGFR+ EngTregs. After resting, cells were stimulated with PMA and Ionomycin in the presence of Golgi-stop for 5 hours before analyzing intracellular cytokine production by flow cytometry.

FIG. 173A depicts an in vivo assessment of the suppressive capabilities of UCB-derived EngTregs against allogeneic effector T cells (Teff) in a xenogeneic GvHD mouse model, and includes a timeline and study design of in vivo study comparing suppression of allogeneic Teff by PBMC-derived and UCB-derived EngTregs in NSG mice. FIG. 173B depicts a survival curve over the duration of the in vivo study. 20% body weight loss is set as the humane study endpoint. The graph has combined data from both UCB donors. FIG. 173C depicts a graph representing changes in body weight for each mouse between DO and study endpoint. Graph has combined data from two donors.

FIG. 174 depicts a survival curve of NSG mice in combined xenoGvHD studies. Data presented are combined results of 2 cord blood cell products against 3 different allogeneic PB-derived CD4 Teffs, respectively. Autologous PB derived EngTregs and mocked-edited cells were included as control. P values were calculated using Log-rank (Mantel-Cox) test.

FIG. 175A depicts gene editing in CD8+ T cells resulting in expression of FOXP3 and conversion of Treg characteristics, and includes a schematic of FOXP3 gene editing in T cells to express MND-driven IL-2 CISC EngTregs. RNP complex containing Cas 9 and FOXP3-specific gRNA was electroporated into T cells followed by AAV transduction. HDR resulted in the allele expressing IL-2 CISC and endogenous FOXP3 driven by MND promoter. FIG. 175B depicts a timeline and key steps to generate IL-2 CISC EngTregs. CD3+ T cells isolated from healthy donors were subjected to gene editing followed by post-editing enrichment and expansion. FIG. 175C depicts a bar graph showing editing efficiency analyzed at day 2 post gene editing. Percent FOXP3+ was analyzed in live singlet CD4+ and CD8+ subsets. FIG. 175D depicts a percent FOXP3+ after enrichment and expansion step as indicated as Day 16 in panel B. FIG. 175E depicts histograms of indicated Treg markers in CD4+ and CD8+ subsets of the cell products. FIG. 175F (left panel) depicts a flow cytometry results of CD8+ mock-edited (top row) cells and EngTregs (bottom row) showing intracellular expression of indicated cytokines in response to PMA and ionomycin stimulation. FIG. 175F (right panel) depicts a graph of relative cytokine positivity compared to mock-edited cells. FIG. 175G depicts a cell trace violet (CTV)-labelled autologous CD4s (left graph) or CD8s (right graph) responders were treated with CD3/CD28 T activator beads at 10:1 bead-to-responder cells and then co-cultured with CD4 or CD8 CISC-EngTregs or mock-edited counterparts at indicated ratios. After a 96-hour incubation, proliferation of CD4+ or CD8+ responders were analyzed by flow cytometry. Percent Suppression=[(% proliferation of responders without suppressors)−(% proliferation of responders with suppressors)]/(% proliferation responders without suppressors)×100.

FIG. 176 depicts a FACS analysis of CD8+ T cells, and generation of CD8+ CISC EngTregs from purified CD8+ T cells: editing efficiency on day 2 post editing.

FIG. 177A depicts CD8 CISC EngTregs post rapa enrichment and expansion, and includes a graph for % FOXP3+ cells and time. FIG. 177B depicts a FACS analysis.

FIG. 178 depicts CD8 CISC EngTregs immunophenotypes in a FACS analysis.

FIG. 179 depicts CD8 CISC EngTregs cytokine production in a FACS analysis.

FIG. 180A depicts a timeline for A2CAR EngTregs production.

FIG. 180B depicts an LV.A2CAR.P2A.LNGFR constructs (LV3350), an AAV3195 construct. FIG. 180C depicts tables listing groups 1-9 for various cells, and schematics for construct 3362 and construct 3407. FIG. 180D depicts a FACS analysis for modified cells.

FIG. 181 depicts CD3 editing: HDR detection at day 3 post-editing in a FACS analysis.

FIG. 182 depicts CD3 editing—HDR detection: analysis in CD4/CD8 subsets in a FACS analysis.

FIG. 183 depicts CD8 editing: HDR analysis in a FACS analysis.

FIG. 184A depicts TRAC/FOXP3 dual editing with Split CISC and A2CAR and includes a 3362 construct, a 3407 construct. FIG. 185B depicts a FACS analysis with cells modified with the constructs depicts in FIG. 184A.

FIG. 185 depicts a FACS analysis with FOXP3 dual editing with Split CISC and A2CAR and includes the 3362 and 3407 constructs shown in FIG. 184A.

FIG. 186A depicts a pRRL_MND.A2CAR.PA2.LNGFR construct for LVA2CAR.CISC EngTregs TCRnull editing. FIG. 186B depicts a FACS analysis with cells modified with the constructs depicts in FIG. 186A.

FIG. 187 depicts LNGFR affinity selection to enrich A2CAR+ cells in a FACS analysis.

FIG. 188 depicts an in vivo study and includes a timeline and example groups. The study uses a similar procedure to the CD4 A2CAR in vivo studies disclosed herein in which EngTregs and PBMC together are injected 1 day after irradiation; mix cells immediately prior to injection. If CD3 derived products are used, CD4/CD8 cells are separated before injection. In the groups, ratios: 1:1, 2:1, 4:1 are used. Proposed A2+ PBMC donor: R003791 for first experiment and R003798 for second experiment with same donors used in the CD4 A2CAR LNGFR study.

FIG. 189 depicts a TABLE including sequences for certain constructs encoding A2 CARs.

FIG. 190A depicts generation of islet specific EngTregs by FOXP3 HDR-editing and LV TCR transduction and includes a timeline of key steps for generating and enriching islet specific EngTregs from primary human CD4+ T cells. T cells were activated with CD3/CD28 beads on day 0 followed by transduction with lentiviral vectors (encoding islet specific TCRs on day 1). On day 7, flow cytometry was used to assess expression of islet specific TCR and Treg markers (mTCR CD25, CD127 CTLA-4 and ICOS). On day 10, islet specific EngTregs were enriched on LNGFR magnetic beads.

FIG. 190B depicts a diagram of FOXP3 locus (top); exons are represented by boxes. The AAV 6 donor template (bottom) was designed to insert the MND promoter, truncated LNGFR coding sequence and P2A (2A) sequence. After successful editing, the MND promoter drives expression of LNGFR and FOXP3.

FIG. 190C depicts representative flow plots (day 7, 4 days post editing) showing co expression of FOXP3 and LNGFR in edited cells (left panel), expression of mTCR, CD25, CD127, CTLA 4 and ICOS gated on LNGFR+ FOXP3+ cells from the left panel.

FIG. 190D depicts representative flow plots (day 10, 7 days post editing) showing purity of LNGFR+ cells post-enrichment on anti-LNGFR magnetic beads. LNGFR− T cells were also collected to serve as controls for the in vitro suppression assays.

FIG. 190E depicts TCR expression and antigen specific proliferation of T cells transduced with islet TCR and include a schematic showing structure of lentiviral islet-specific TCR including variable region of human islet-specific TCR (huV-alpha and huV-beta) and constant region of murine TCR (muV-alpha and muV-beta).

FIG. 190F depicts validation of islet-specific TCR expression in human CD4+ T cells transduced with islet-specific TCRs. CD4+ T cells were isolated, activated with CD3/CD28 beads, and transduced with each lentiviral islet-specific TCR. Flow plots show mTCR expression in CD4+ T cells at 7 days post transduction using an antibody specific for the mouse TCR constant region.

FIG. 190G depicts proliferation of CD4+ T cells transduced with islet TCR in the presence of APC and their cognate peptide. TCR-transduced CD4+ T cells were labeled with cell trace violet and then co cultured with their cognate peptide (or irrelevant peptide) and APC (irradiated PBMC) for 4 days. Flow plots show cell proliferation as CTV dilution.

FIG. 191A depicts islet-specific EngTregs suppress antigen-induced Teff proliferation and includes a schematic of direct suppression of Teff by EngTregs with specificity for the same islet antigen. Shown here both the EngTregs and Teff are expressing T1D5-2 TCR, specific for IGRP₃₀₅₋₃₂₄.

FIG. 191B depicts representative histograms showing proliferation of T1D5-2 Teff (measured by CTV dilution) in the presence of either anti-CD3/CD28 antibody coated beads (top row) or cognate peptide (IGRP₃₀₅₋₃₂₄) and APC (bottom row) and the EF670-labelled EngTregs or controls. Histograms were gated on EF670− cells.

FIG. 191C depicts percent suppression of CD3/CD28 bead-induced Teff proliferation by poly EngTregs, LNGFR− T cells and islet-specific EngTregs either T1D5-2 (left), PPI76 (middle) or GAD65 (right).

FIG. 191D depicts percent suppression of antigen-induced Teff proliferation by poly EngTregs, LNGFR− T cells and islet-specific EngTregs either T1D5-2 (left), PPI76 (middle) or GAD65 (right); the cognate peptides were IGRP₃₀₅₋₃₂₄, PPI₇₆₋₉₀ and GAD65₂₆₅₋₂₈₄, respectively. For FIG. 191C and FIG. 191D, data are represented as mean±SD of three independent experiments using cells generated from three different healthy donors. P-values were calculated using a paired two-tailed Student t test (*P<0.05 and **P<0.01).

FIG. 191E depicts a timeline and key steps for production of islet specific EngTregs and Teff and the in vitro suppression assay. Teff were generated by TCR transduction of CD4+ T cells after activation with CD3/CD28 beads. Teff were expanded and harvested at day 15. Procedure for EngTregs production is described in FIG. 109A. Teff were co-cultured with or without EngTregs or LNGFR T cells in the presence of either APC (irradiated autologous PBMC) and various peptides or in the presence of CD3/CD28 beads. Teff and EngTregs or LNGFR− T cells were labeled with cell trace violet (CTV) and EF670 respectively, prior to co-culture. After 3 or 4 days of incubation, cells were harvested, stained, and analyzed by flow.

FIG. 192A depicts islet-specific EngTregs suppress antigen-induced Teff cytokine production and includes representative flow plots showing Teff cytokine production (TNF-alpha, IL-2 and IFNγ) and activation (CD25 expression) in an antigen-specific suppression assay. T1D5-2 Teff in the presence of T1D5-2 cognate peptide IGRP₃₀₅₋₃₂₄ and APC were cultured alone or with polyclonal EngTregs, LNGFR− T cells, or T1D5-2 EngTregs.

FIG. 192B depicts percent suppression of antigen-induced T1D5-2 Teff production of TNFα (left) IL-2 (middle) and IFNγ (right) by poly EngTregs LNGFR− T cells and islet-specific T1D5-2 EngTregs.

FIG. 192C depicts percent suppression of antigen-induced T1D5-2 Teff expression of CD25 by poly EngTregs, LNGFR− T cells and islet-specific T1D5-2 EngTregs. For FIG. 192B and FIG. 192C, data are represented as mean±SD of four independent experiments using cells generated from four different healthy donors. P values were calculated using a paired two tailed Student t test (*P<0.05, **P<0.01 and ***P<0 001).

FIG. 193A depicts islet-specific EngTregs suppress bystander Teff proliferation and includes a schematic of bystander suppression of Teff by EngTregs with specificity for different islet antigens. Shown here the EngTregs expresses T1D4 TCR specific for IGRP₂₄₁₋₂₆₀, and the Teff express T1D5-2 TCR specific for IGRP₃₀₅₋₃₂₄.

FIG. 193B depicts representative histograms showing proliferation of T1D5-2 Teff (measured by CTV dilution) in the presence of either IGRP₃₀₅₋₃₂₄ peptide (top panel) or mixture of IGRP₃₀₅₋₃₂₄ and IGRP₂₄₁₋₂₆₀ peptides (bottom row) plus APC and either T1D5-2 EngTregs, T1D4 EngTregs or poly EngTregs. EngTregs were labeled with EF670 and histograms were gated on EF670− cells.

FIG. 193C depicts percent suppression of T1D5-2 Teff proliferation by poly EngTregs, T1D5-2 EngTregs or T1D4 EngTregs in the presence of a mixture of IGRP₃₀₅₋₃₂₄ and IGRP₂₄₁₋₂₆₀ peptides plus APC.

FIG. 193D depicts representative histograms showing proliferation of T1D5-2 Teff (measured by CTV dilution) in the presence of either IGRP₃₀₅₋₃₂₄ peptide (top panel) or mixture of IGRP₃₀₅₋₃₂₄ and GAD₂₆₅₋₂₈₄ peptides (bottom row) plus APC and poly EngTregs and GAD265 EngTregs. EngTregs were labeled with EF670 and histograms were gated on EF670− cells.

FIG. 193E depicts percent suppression of proliferation of T1D5-2 Teff by poly EngTregs or GAD265 EngTregs in the presence of APC and mixture of IGRP₃₀₅₋₃₂₄ and GAD₂₆₅₋₂₈₄ peptides plus APC.

FIG. 193F depicts percent suppression of T1D5-2 Teff cytokine production by T1D5-2 Teff by poly EngTregs, T1D5-2 EngTregs or T1D4 EngTregs in the presence of APC and mixture of IGRP₃₀₅₋₃₂₄ and IGRP₂₄₁₋₂₆₀ peptides.

FIG. 193G depicts percent suppression for T1D5-2 Teff CD25 expression by poly EngTregs, T1D5-2 EngTregs or T1D4 EngTregs in the presence of APC and mixture of IGRP₃₀₅₋₃₂₄ peptide and IGRP₂₄₁₋₂₆₀ peptide. For FIG. 193C, FIG. 193E, FIG. 193F and FIG. 193G, data are provided as the mean±SD of three independent experiments using cells generated from three different healthy donors. P values were calculated using a paired two tailed Student t test (*P<0.05, P<0.01 and P<0.005). LNGFR− T cells with either T1D5-2 TCR or T1D4 TCR were used as a negative control for all three experiments and did not show any significant suppression.

FIG. 193H depicts islet-specific EngTregs show comparable suppression on CD3/CD28 bead induced Teff proliferation and includes representative flow plots showing mTCR expression in FOXP3-edited cells transduced with no TCR (−), T1D4 TCR or T1D5-2 TCR. Edited cells were stained at day 7 and were gated on Live, CD3+, CD4+, LNGFR+, FOXP3+.

FIG. 193I depicts representative histograms showing proliferation of T1D5-2 Teff in CD3/CD28 bead suppression assay performed in parallel with bystander suppression assay in FIG. 193B and FIG. 193C. T1D5-2 Teff were incubated with CD3/CD28 beads with no Treg (−), polyclonal EngTregs, T1D5-2 EngTregs, or T1D4 EngTregs.

FIG. 193J depicts percent suppression of CD3/CD28 bead induced-T1D5-2 Teff proliferation by poly EngTregs, T1D5-2 EngTregs, or T1D4 EngTregs in (FIG. 193I).

FIG. 193K depicts representative histograms showing T1D5-2 Teff proliferation in CD3/CD28 bead suppression assay performed in parallel with bystander suppression assay in FIG. 193D and FIG. 193E. T1D5-2 Teff were incubated with CD3/CD28 beads with no Treg (−), poly EngTregs, or GAD265 EngTregs.

FIG. 193L depicts percent suppression of CD3/CD28 bead induced-T1D5-2 Teff proliferation by poly EngTregs or GAD265 EngTregs in FIG. 193K. For FIG. 193J and FIG. 193L, data are represented as the mean±SD of three independent experiments using cells generated from three different healthy donors. P-values were calculated using a paired two-tailed Student t test.

FIG. 193M depicts representative histograms showing islet specific EngTregs suppression of bystander Teff cytokine production and includes representative histograms showing T1D5-2 Teff production of TNFα in antigen-specific bystander suppression assay. Columns are the same as those labelled in FIG. 193M.

FIG. 193N depicts representative histograms showing T1D5-2 Teff production of IL2 in antigen-specific bystander suppression assay. Columns are the same as those labelled in FIG. 193M.

FIG. 193O depicts representative histograms showing T1D5-2 Teff production of IFNγ in antigen-specific bystander suppression assay. Columns are the same as those labelled in FIG. 193M.

FIG. 193P depicts representative histograms showing T1D5-2 Teff expression of CD25 in antigen-specific bystander suppression assay. Columns are the same as those labelled in FIG. 193M. For FIGS. 193M-193P, T1D5-2 Teff were co-cultured with no Treg poly EngTregs, T1D5-2 EngTregs or T1D4 EngTregs in the presence of APC and either IGRP₃₀₅₋₃₂₄ peptide alone or a mixture of IGRP₃₀₅₋₃₂₄ and IGRP₂₄₁₋₂₆₀ peptides.

FIG. 194A depicts islet-specific EngTregs suppress polyclonal islet-specific Teff derived from T1D PBMC, and includes a timeline and key steps for production of islet-specific EngTregs, polyclonal islet specific Teff, and monocyte derived DC (mDC) from PBMC from T1D donor, and the in vitro suppression assay.

FIG. 194B depicts representative histograms showing proliferation of polyclonal islet Teff (measured by CTV dilution) in the presence of either CD3/CD28 beads (top panel) or islet-specific antigens (9 islet specific peptides monocyte derived DC (mDC)) (bottom row) and either T1D2 EngTregs, 4.13 EngTregs, LNGFR− T cells or poly EngTregs. EngTregs were labeled with EF670 and histograms were gated on EF670− cells

FIG. 194C depicts percent suppression of CD3/CD 28 induced proliferation of polyclonal islet Teff by T1D2 EngTregs, 4.13 EngTregs, LNGFR− T cells or poly EngTregs.

FIG. 194D depicts percent suppression of antigen-induced proliferation of polyclonal islet Teff by T1D2 EngTregs, 4.13 EngTregs, LNGFR− T cells or poly EngTregs. Antigen stimulation by pool of 9 islet specific peptides in the presence of mDC. Data are provided as the mean±SD of three independent experiments using cells generated from three different T1D donors. P values were calculated using a paired two-tailed Student t test (* P<0.05 **P<0.01 and ***P<0.0001). Co-culture in the presence of mDC and DMSO was included as a negative control and showed no significant proliferation of Teff.

FIG. 194E depicts expansion of islet-specific T cells of multiple specificities derived from T1D PBMC, and includes a timeline and key steps of peptide stimulation to expand islet-specific T cells. CD4+CD25− T cells isolated from T1D donor were stimulated with HLA-DR0401 restricted 9 islet peptides specific for GAD65 (5), IGRP (3), and PPI (1) and irradiated autologous APC (CD4−CD25+) followed by tetramer staining at day 12 to 14. T cells were cultured without IL-2 until day 7, and then expanded with IL-2 at 2-3 days of interval.

FIG. 194F depicts representative flow plots showing tetramer+ T cells specific for individual antigenic peptides. Staining with no tetramer was included as a negative staining result. Cells were gated on CD4+ T cells and each percentage indicates the level of tetramer staining above background.

FIG. 194G depicts percent tetramer+ population in CD4+ T cells measured and combined from 5 different experiments using 3 different T1D donors after 12-14 days of in vitro peptide stimulation. Each bar indicates the percentage of CD4+ T cells specific for each islet antigenic peptide. Each dot represents a different experiment.

FIG. 194H depicts islet-specific EngTregs are superior at suppressing polyclonal islet-specific Teff than tTreg, and includes representative histograms showing proliferation of polyclonal islet Teff in the presence of either anti-CD3/CD28 antibody coated beads (Top row) or mDC and a pool of 9 islet-specific peptides (Bottom row) performed in parallel. Polyclonal islet Teff were cultured with no Treg (−), T1D2 LNGFR−, T1D2 EngTregs, or tTreg. tTreg were sorted by CD4+CD25+CD127− and cultured in the same way as EngTregs. tTreg were activated with CD3/CD28 beads for 2 days, expanded, and harvested at day 10. All the cells used for suppression assays are autologous and prepared from a T1D donor. Co-culture in the presence of monocyte-derived DC (mDC) and DMSO was included as a negative control and showed no significant proliferation of Teff.

FIG. 194I depicts percent suppression of CD3/CD28 bead induced-proliferation of polyclonal islet Teff by T1D2 LNGFR−, T1D2 EngTregs, or tTreg.

FIG. 194J depicts percent suppression of antigen induced-proliferation of polyclonal islet Teff by T1D2 LNGFR−, T1D2 EngTregs, tTreg.

FIG. 195A depicts islet specific EngTregs inhibit APC maturation and utilize both cell contact dependent and independent mechanisms to suppress Teff, and include a schematic of transwell suppression assay: upper and lower chamber separated by permeable membrane.

FIG. 195B depicts percent suppression of proliferation of polyclonal islet specific Teff measured by CTV dilution in lower chamber (left panel) or upper chamber (right panel). Polyclonal islet Teff were co cultured with T1D2 EngTregs as a positive control. Data are provided as the mean±SEM of three independent experiments using cells generated from three different T1D donors. ***P<0.001, **P<0.01, *P<0.05, as determined by paired t-test.

FIG. 195C depicts a timeline and key steps for DC maturation and APC modulation assay.

FIG. 195D depicts normalized CD86 MFI on mDC. Autologous matured mDC with HLA DR0401 were co cultured with T1D2 EngTregs or LNGFR− T cells in the presence of IGRP₃₀₅₋₃₂₄ peptide for 2 days. MFI of CD86 on DCs were normalized by MFI of DC only condition. Data are provided as the mean SD of three independent experiments using cells generated from three different healthy donors. *P<0.05, as determined by paired t-test.

FIG. 195E depicts representative histograms showing proliferation of polyclonal islet-specific Teff co-cultured with islet specific antigens (10Ags including IGRP₃₀₅₋₃₂₄) and mDC in the presence of T1D2 EngTregs with addition of exogenous human IL2 (0.1 IU/ml). Teff and EngTregs were labeled with CTV and EF670, respectively, before the co-culture and CTV dilution was measured as proliferation.

FIG. 195F depicts percent suppression on Teff proliferation shown in FIG. 195E. % Suppression was calculated separately in the absence or presence of exogenous human IL2. Data are provided as the mean±SEM of three independent experiments using cells generated from three different T1D donors. Ns, not significant, as determined by paired t-test.

FIG. 195G depicts islet-specific EngTregs show both contact dependent and independent bystander suppression, and includes generation of polyclonal islet-specific Teff to investigate mechanisms for bystander suppression by islet specific EngTregs. CD4+ CD25− T cells isolated from T1D donor were stimulated with HLA-DR0401 restricted 9 islet peptides specific for GAD65₁₁₃₋₁₃₂, GAD₂₆₅₋₂₈₄, GAD₂₇₃₋₂₉₂, GAD₃₀₅₋₃₂₄, IGRP₁₇₋₃₆, IGRP₂₄₁₋₂₆₀, PPI₇₆₋₉₀, ZNT8₂₆₆₋₂₈₅ and irradiated autologous APC (CD4−CD25+) followed by tetramer staining at day 14 or 15. T1D2 TCR specific IGRP₃₀₅₋₃₂₄ peptide was excluded for Teff expansion. Representative flow plots showing tetramer T cells specific for individual antigenic peptides. Staining with no tetramer was included as a negative staining result. Cells were gated on CD4+ T cells and each percentage indicates the level of tetramer staining above background.

FIG. 195H depicts percent tetramer population in CD4+ T cells measured and combined from 3 different T1D donors after 14-15 days of in vitro peptide stimulation. Each bar indicates the percentage of CD4+ T cells specific for each islet antigenic peptide. Each dot represents a different T1D donor.

FIG. 195I depicts representative histograms showing proliferation of polyclonal islet-specific Teff at lower well (top) or upper well (lower). mDC loaded with a pool of islet peptides (10 Ags including IGRP₃₀₅₋₃₂₄) were plated in both lower and upper well. Polyclonal islet-specific Teff or/and T1D2 EngTregs were added in lower or/and upper well as indicated.

FIG. 195J depicts islet-specific EngTregs inhibit CD86 expression on dendritic cells, and includes autologous monocytes restricted to HLA-DR0401 were matured into DC with GM-CSF/IL-4 and IFNg/CL075. Matured DC were co-cultured with CTV-labeled EngTregs or LNGFR− T cells expressing islet-TCR in the presence of cognate peptide. After 2 days of incubation, cells were harvested, stained, analyzed by flow.

FIG. 195K depicts representative data showing MFI of CD86 on DC co-cultured with T1D2 EngTregs or LNGFR− T cells.

FIG. 195L depicts bar histograms showing normalized expression level of CD86 on DC co-cultured with T1D4 EngTregs or LNGFR− T cells in the presence of IGRP₂₄₁-260 peptide (left) or with PPI76 EngTregs or LNGFR− T cells in the presence of PPI₇₆₋₉₀ peptide (right).

FIG. 196A depicts islet-specific EngTregs with low functional avidity exhibit superior suppression of polyclonal islet-specific Teff derived from T1D PBMC, and includes a graph showing peptide dose response of T cells expressing T1D2, T1D5-1, or T1D5-2 TCR. CD4+ T cells transduced with T1D2, T1D5-1 or T1D5-2 TCR were co-cultured with APC in the presence of various concentration of their cognate peptide, IGRP₃₀₅₋₃₂₄ for 4 days. T cells were labeled with CTV before the co-culture and cell proliferation was measured by CTV dilution.

FIG. 196B depicts representative histograms showing proliferation of polyclonal islet Teff in the presence of islet-specific antigens (10 islet-specific peptides+monocytederived DC (mDC)) and either T1D2, T1D5-1, or T1D5-2 EngTregs. Polyclonal islet Teff and EngTregs were labeled with CTV and EF670, respectively and cell proliferation was measured as CTV dilution.

FIG. 196C depicts percent suppression of antigen-induced proliferation of polyclonal islet Teff by T1D2, T1D5-1, or T1D5-2 EngTregs. Data are normalized by suppressive activity obtained from suppression assay setup in parallel using CD3/CD28 beads. Suppressive activity was calculated as (% suppression/% the lowest suppression). Normalization of antigen-specific suppression was calculated as (% suppression from antigen-specific assay/suppressive activity).

FIG. 196D depicts generation of polyclonal islet specific Teff derived from T1D PBMC, and includes CD4+ CD25− T cells isolated from T1D donor were stimulated with HLA-DR0401 restricted 10 islet peptides specific for GAD65₁₁₃₋₁₃₂, GAD₂₆₅₋₂₈₄, GAD₂₇₃₋₂₉₂, GAD₃₀₅₋₃₂₄, IGRP₁₇₋₃₆, IGRP₂₄₁₋₂₆₀, PPI₇₆₋₉₀, ZNT8₂₆₆₋₂₈₅ and irradiated autologous APC (CD4−CD25+) followed by tetramer staining at day 14 or 15. Representative flow plots showing tetramer+ T cells specific for individual antigenic peptides. Staining with no tetramer was included as a negative staining result. Cells were gated on CD4+ T cells and each percentage indicates the level of tetramer staining above background.

FIG. 196E depicts percent tetramer population in CD4+ T cells measured and combined from 5 different experiments using 4 different T1D donors after 14-15 days of in vitro peptide stimulation. Each bar indicates the percentage of CD4+ T cells specific for each islet antigenic peptide. Each dot represents a different T1D donor.

FIG. 196F depicts normalization of antigen-specific suppression of polyclonal islet Teff, and includes representative histograms showing proliferation of polyclonal islet Teff in the presence of anti-CD3/CD28 antibody coated beads or mDC and a pool of 9 islet-specific peptides (Bottom row) performed in parallel in the presence of mDC and a pool of 10 islet-specific peptides shown in FIG. 196B and FIG. 196C.

FIG. 196G depicts percent suppression of CD3/CD28 bead induced-proliferation of polyclonal islet Teff by T1D2, T1D5-1, or T1D5-2 EngTregs shown in FIG. 196F.

FIG. 197A depicts generation of murine islet-specific EngTregs by gene editing in BDC2.5 CD4+ T cells and includes a diagram of AAV 5 packaged, MND LNGFR p2A knock-in donor template for use in FOXP3 HDR editing. Exons are represented by numbered boxes, FOXP3 homology arms are indicated. After successful editing, the MND promoter drives expression of endogenous murine FOXP3 protein and cis-linked LNGFR surface expression.

FIG. 197B depicts a schematic showing the experimental timeline for FOXP3 gene editing, cell analysis, and enrichment of edited LNGFR cells.

FIG. 197C depicts representative flow plots (from one of four independent experiments) showing LNGFR expression in mock-edited control cells (left) and cells edited with RNP and AAV donor template pre- (middle) and post- LNGFR+ column-enrichment (right).

FIG. 197D depicts representative flow cytometry histogram (from one of two independent experiments) showing the expression of Treg associated markers for the indicated cell populations.

FIG. 197E depicts bar graphs showing MFI for Treg associated markers on EngTregs, or mock edited cells. Error bars show±SD. P values were calculated using an unpaired T test comparing EngTregs and mock edited cells.

FIG. 197F depicts a schematic of in vitro suppression assays performed using BDC2.5 CD4+ Teff cells and mock control, BDC2.5 tTreg or EngTregs cells.

FIG. 197G depicts representative flow plots (from one of three independent experiments) showing CTV labeled BDC2.5 CD4+ Teff co-cultured with the indicated cells 4 days post stimulation.

FIG. 197H depicts a graph showing the percent suppression of BDC2.5 CD4+ Teff proliferation by the indicated Treg co culture at varying ratios of Teff Treg suppression 100 normalized suppression] normalized suppression 100/proliferation of Teff only condition×Teff proliferation in the presence of Treg.

FIG. 198A depicts islet specific, but not polyclonal, EngTregs prevent T1D onset in vivo, and includes a schematic showing the experimental timeline for murine diabetes prevention studies.

FIG. 198B depicts a graph showing diabetes-free survival of recipient NSG mice after infusion of islet-specific Teff in the presence of the indicated co-transferred cell populations. Data are combined from two independent experiments; ****, P<0.0001, calculated using a log rank (Mantel-Cox) test comparing the BDC2.5 tTreg or EngTreg groups vs. the mock-edited control group.

FIG. 198C depicts at left panel including representative flow plots of lymphocytes isolated from the pancreas in diabetes-free NSG recipient mice on day 49 after BDC2.5 CD4 Teff infusion. Upper and lower panels show data for recipients of BDC2.5 tTreg vs. BDC2.5 EngTreg, respectively. Predecessor gates for flow panels are indicated at the top of each column. Right panel, histograms show FOXP3 expression within the indicated (color coded) flow gates.

FIG. 198D depicts representative flow plots showing LNGFR expression in the indicated (top of column) edited CD4 T cells derived from NOD (polyclonal; top row) and NOD BDC2.5 mice (islet specific; bottom row).

FIG. 198E depicts a graph showing diabetes-free survival in recipient NSG mice following infusion of islet specific Teff in the presence co transferred mock edited, or polyclonal or islet specific EngTregs or tTreg cells. Combined data from two independent experiments are shown; ****P<0.0001, determined using the Mantel Cox log rank test comparing BDC2.5 tTreg or EngTregs vs. polyclonal tTreg or EngTregs, respectively. All flow plots are representative of at least two independent experiments.

### DETAILED DESCRIPTION

Provided herein are compositions and methods for editing of more than one genomic locus in a cell using a chemical-inducible signaling complex (CISC) system, editing the TRAC locus of a cell's genome (including promoter capture methods, TCR/CAR knock-in methods, and methods of hijacking the TRAC gene with a promoter). Also provided herein are compositions and methods for suppressing proliferation of T effector cells using genetically modified/engineered airT cells, and methods of treating an autoimmune, allergic, and/or inflammatory disease in a subject using genetically modified/engineered airT cells. Methods for designing TCRs of particular properties, e.g., particular avidity are also provided here.

### Editing of More than One Genetic Locus in a Cell Using a Chemical-Inducible Signaling Complex (CISC) System

Some embodiments of the methods and compositions provided herein relate to efficient editing of more than one genetic locus in a cell using a chemical-inducible signaling complex (CISC) system. In some embodiments, a CISC complex comprises a first CISC component comprising a first extracellular binding domain, a transmembrane domain, and a first signaling domain; and a second CISC component comprising a second extracellular binding domain, a transmembrane domain, and a second signaling domain, such that the first and second CISC components (e.g., via the first and second extracellular binding domains) are capable of binding a CISC inducer molecule (e.g., a small molecule such as rapamycin or its analogs). In some embodiments, a CISC complex system comprises a CISC complex comprising a first and second CISC component and further a third CISC component that is different from the first and second CISC components and is capable of specifically binding to the CISC inducer molecule. In some embodiments, the third CISC component is soluble and does not comprise a transmembrane domain or an extracellular domain. In some embodiments, the soluble third CISC component does not comprise a secretory peptide and is localized in the cytoplasm of the cell. In some embodiments, the first extracellular binding domain comprises FKBP, the second extracellular binding domain comprises FRB. In some embodiments, the third CISC component comprises soluble or cytosolic FRB. Co-expression of two (e.g., a first and second) transmembrane CISC components that are capable of dimerizing in the presence of a CISC inducer molecule (e.g., rapamycin or a rapalog) is useful for controlling signal transduction by providing the dimerizing molecule. However, inducer molecules may also exert undesired effects on cellular metabolism. For example, intracellular rapamycin can bind to a free FKBP protein, and an FKBP-rapamycin complex can then stimulate the mechanistic target of rapamycin (mTOR), which inhibits mRNA translation and cellular growth. Expression of a third CISC component, such as soluble FRB, in the cytoplasm, allows the third CISC component to serve as a decoy receptor for rapamycin or other CISC inducer molecules, thereby preventing or reducing undesired effects of the CISC inducer molecule on cellular physiology. CISC components and methods useful in embodiments of the cells and methods provided herein are provided in WO 2019/210057, which is incorporated by reference herein in its entirety.

In some embodiments, a first polynucleotide comprises a nucleic acid encoding a first CISC component as provided herein. In some embodiments, a second polynucleotide comprises a nucleic acid encoding a second CISC component as provided here. In some embodiments, a third polynucleotide comprises a nucleic acid encoding a third CISC component as provided herein. In some embodiments, a first polynucleotide comprising a nucleic acid encoding a first CISC component further comprises a first regulatory element (e.g., a first promoter). In some embodiments, a second polynucleotide comprising a nucleic acid encoding a second CISC component further comprises a second regulatory element (e.g., a second promoter). In some embodiments, a first polynucleotide comprising a nucleic acid encoding a third CISC component further comprises a third regulatory element (e.g., a third promoter). In some embodiments, a polynucleotide comprising nucleic acid encoding a first, second and/or third components does not comprise any regulatory elements, and relies on or is functionally linked to one or more genomic regulatory elements upon insertion into the genome. In some embodiments, the first and second, first and third, second and third, or first, second and third CISC components can be inserted into the same locus. In some embodiments, the first and second, first and third, second and third, or first, second and third CISC components can be on the same polynucleotide. In some embodiments, the first and second polynucleotides are the same and comprise both the nucleic acid encoding the first CISC component and the nucleic acid encoding the second CISC component. In some embodiments, the first and third polynucleotides are the same and comprise both the nucleic acid encoding the first CISC component and the nucleic acid encoding the third CISC component. In some embodiments, the second and third polynucleotides are the same and comprise both the nucleic acid encoding the second CISC component and the nucleic acid encoding the third CISC component. In some embodiments, a polynucleotide comprising nucleic acids encoding first and second CISC components also comprises a nucleic acid encoding a third CISC component as provided herein. In some embodiments, any two or three of the first, second, and third CISC components share a common or are functionally linked to the same regulatory element.

In some embodiments, a first CISC component and the second CISC component are configured such that when expressed in a cell, they are capable of dimerizing in the presence of an inducer molecule (e.g., rapamycin or a rapalog) to generate a signaling-competent CISC.

In some embodiments, a method of editing more than one genetic loci (e.g., a first locus and a second locus) in a cell comprises providing to a cell any or all of: a first polynucleotide comprising a nucleic acid encoding a first CISC component (and optionally a third CISC component) as provided herein such that the first polynucleotide is inserted in the first locus; and a second polynucleotide comprising a nucleic acid encoding a second CISC component (and optionally a third CISC component) as provided herein such that the second polynucleotide is inserted in the second locus. In some embodiments, first and second CISC components (and optionally the third CISC components) are comprised on the same polynucleotide and are inserted in the same locus (e.g., the Foxp3 gene/locus or the TRAC gene or locus/gene). A particular locus may be targeted by designing endonucleases that cut at a particular location and by inserting sequences (e.g., homology arms) that flank one or more nucleic acids that encode a CISC component. It is to be understood, that more than two loci can be edited using the methods disclosed herein by insertion of one or more of any one of the first, second, and third CISC components provided herein.

In some embodiments, a method of editing one or more genetic/genomic loci in a cell comprises: (a) contacting the cell with (i) a first nucleic acid comprising a polynucleotide encoding a first CISC component comprising a first extracellular binding domain, first transmembrane domain, and first intracellular signaling domain (ii) a second nucleic acid comprising a second polynucleotide encoding a second CISC component comprising a second extracellular binding domain, a transmembrane domain, and a second intracellular signaling domain, (iii) a first endonuclease, or nucleic acid encoding the first endonuclease, that can cleave a first nucleotide sequence within a first locus, (iv) a second endonuclease, or nucleic acid encoding the second endonuclease, that can cleave a second nucleotide sequence within a second locus, such that the first polynucleotide or fragment thereof is incorporated into the first locus, and the second polynucleotide or fragment there is is inserted in the second locus; and (b) contacting the cells with a dimerization agent (e.g., a ligand) that binds to the first extracellular binding domain of the first CISC component and the second extracellular binding domain of the second CISC component, such that the first intracellular signaling domain and second intracellular signaling domain dimerize in cells expressing both the first CISC component and the second CISC component, resulting in signal transduction through interactions between the first and second intracellular signaling domains. In some embodiments, a first polynucleotide further comprises homology arms targeting a first locus. In some embodiments, a second polynucleotide further comprises homology arms targeting a second locus. In some embodiments, the first CISC component comprises an FKBP extracellular domain, a transmembrane domain, and an IL2RB intracellular signaling domain, and the second CISC component comprises an FRB extracellular domain, a transmembrane, and an IL2RG intracellular signaling domain, wherein the first and second CISC components dimerize in the presence of rapamycin or a rapalog, allowing the cell to respond to rapamycin in a manner similar to IL-2 stimulation. In some embodiments, the first CISC component comprises an FKBP extracellular domain, a transmembrane domain, and an IL2RG intracellular signaling domain, and the second CISC component comprises an FRB extracellular domain, a transmembrane, and an IL2RB intracellular signaling domain, wherein the first and second CISC components dimerize in the presence of rapamycin or a rapalog, allowing the cell to respond to rapamycin in a manner similar to IL-2 stimulation. In some embodiments, a transmembrane domain of CISC component comprises a transmembrane domain corresponding to the transmembrane domain of the intracellular signaling domain of the CISC component (e.g., a CISC component comprising an IL2RB intracellular domain comprises a transmembrane domain derived from IL2RB). In some embodiments, both the first and second CISC components comprise a transmembrane domain derived from the same protein (e.g., IL2RB). In some embodiments, the transmembrane domain of a CISC component is derived from a different protein than the intracellular signaling domain of the CISC component (e.g., a CISC component comprising an IL2RB intracellular signaling domain comprises a transmembrane domain that is not derived from a full-length IL2RB protein).

The extracellular domains of CISC components can be any domains that can dimerize upon binding to an agent. Such an agent may be a small molecule (e.g., rapamycin) or a protein (e.g., lysozyme), or any other agent. The extracellular domains may be domains that bind to a small molecule, or to a protein, e.g., antibody binding domains or fragments thereof that dimerize upon binding to the ligand.

In some embodiments, the first locus is a FOXP3 locus, and the second locus is a TRAC locus. In other embodiments, the first locus is a TRAC locus, and the second locus is a FOXP3 locus. In some embodiments, the first and second loci are independently selected from a FOXP3 locus, a TRAC locus, an AAVS1 locus, and a ROSA26 locus. In some embodiments, both nucleic acid molecules are inserted into different alleles of the same locus, which is selected from a FOXP3 locus, a TRAC locus, an AAVS1 locus, and a ROSA26 locus.

In some embodiments, a method further comprises separating cells that bind to the ligand to a greater extent that other cells. In some embodiments, the first polynucleotide further comprises one or more regulatory elements and/or a first payload. In some embodiments, a first polynucleotide encodes a first CISC component and a regulatory element but not a payload. A payload, as described herein, is a protein or RNA encoded by a nucleic acid. In some embodiments, the second polynucleotide further comprises one or more regulatory elements and/or a nucleic acid encoding a second payload. In some embodiments, (i) a first polynucleotide comprises an MND promoter operably linked to a nucleic acid encoding (a) a first CISC component comprising an FRB extracellular domain, a transmembrane domain, and an IL2RB intracellular signaling domain, and (b) a third CISC component comprising a soluble FRB domain, wherein the MND promoter is inserted upstream of the first coding exon or first codon of the endogenous FOXP3 gene, and downstream of (e.g., 10 to 10,000 bp downstream from) a TSDR in the endogenous FOXP3 locus of the cell; and (ii) a second polynucleotide comprises an MND promoter operably linked to a nucleic acid encoding (a) a second CISC component comprising an FKBP extracellular domain, a transmembrane domain, and an IL2RG intracellular domain, (b) an exogenous TCRβ, and (c) an exogenous TRAV and TRAJ amino acid sequences, wherein the second nucleic acid is inserted into the TRAC locus upstream from nucleic acid encoding the TCRα constant region amino acid sequence, wherein the inserted MND promoter is controls transcription of the exogenous TCRβ and a TCRα comprising the exogenous TRAV and TRAJ amino acid sequences and the endogenous TCRα amino acid sequences. In some embodiments, the first and/or second endonuclease or nucleic acid encoding them are provided, along with one or more gRNAs to target the first and/or second locus. In some embodiments, an endonuclease is an RNA-guided nuclease. In some embodiments, an RNA-guided nuclease is a CRISPR/Cas nuclease. In some embodiments, a CRISPR/Cas nuclease is a Type I Cas nuclease. In some embodiments, a CRISPR/Cas nuclease is a Type II Cas nuclease. In some embodiments, a CRISPR/Cas nuclease is a Type III Cas nuclease. In some embodiments, a CRISPR/Cas nuclease is a Type V Cas nuclease. In some embodiments, the CRISPR/Cas nuclease is selected from the group consisting of Cas9, SaCas9, CjCas9, xCas9, C2C1, Casl3a/C2c2, C2c3, Casl3b, Cpf1, and variants thereof.

In some embodiments, a first and/or second polynucleotide as described herein further comprises a payload or nucleic acid encoding a polypeptide or a functional fragment thereof. Non-limiting examples of a payload or nucleic acid encoding a polypeptide or a functional fragment thereof include a TCR, CAR, Foxp3 or a functional fragment thereof. In some embodiments, a first and/or second polynucleotide comprises one or more regulatory elements (e.g., one or more promoters). In some embodiments, a first and/or second polynucleotide comprises one or more regulatory elements (e.g., one or more promoters) without comprising a payload or nucleic acid encoding a polypeptide or a functional fragment thereof (unless the nucleic acid encoding a polypeptide constitutes a targeting sequence or homology arm used to target a particular locus in a genome). For example, a targeting sequence or homology arm may be a Foxp3 targeting arms that comprises a nucleic acid encoding and exon of Foxp3, but that does not actually require that the coding region or functional fragment of a FOXP3 gene. In some embodiments, a first and/or second polynucleotide as described herein further comprises a payload or nucleic acid encoding a polypeptide or a functional fragment thereof, and one or more regulatory elements. For example, a first polynucleotide may comprise a nucleic acid encoding a first CISC component (and optionally a third CISC component as described herein) and a promoter (e.g., MND promoter) for insertion into a first locus (e.g., Foxp3 locus), and a second polynucleotide may comprise a nucleic acid encoding a second CISC component (and optionally a third CISC component as described herein) and a nucleic acid encoding a polypeptide or a functional fragment thereof (e.g., a TCR or a CAR) for insertion into a second locus (e.g., TRAC locus). In some embodiments, more than one payload is delivered to one or more loci. For example, a nucleic acid encoding Foxp3 or comprising only a regulatory element (e.g., a promoter) without a nucleic acid encoding Foxp3 may be inserted into the Foxp3 locus and a nucleic acid encoding a TCR or CAR may be inserted in the TRAC locus. It should be understood that any gene or any locus may be used in any one of the methods disclosed herein that comprise insertion of one or more polynucleotides comprising nucleic acid encoding any one or more of a first, second, or third CISC component.

In some embodiments, a FOXP3 locus/gene and TRAC locus/gene is in a cell, e.g., a T cell. In some embodiments, a T cell is a Treg cell (e.g., Foxp+, Tr1, Th3, CD4+, CD8+CD8+CD28−, or Qa-1 restricted), and is edited using the CISC components as provided herein. In some embodiments, a cell as provided herein is an engineered cell. In some embodiments, an engineered cell is a cell in which one or more genes/loci are manipulated or edited (e.g., to stabilize expression of one or more genes). In some embodiments, an engineered cell comprises an edited Foxp3 gene/locus, e.g., obtained by inserting a promoter (in some embodiments, downstream of (e.g., 10 to 10,000 bp downstream from) one or more regulatory elements like the TSDR, and/or upstream from the first coding exon or codon).

### Methods of Editing TRAC Locus in a Cell's Genome

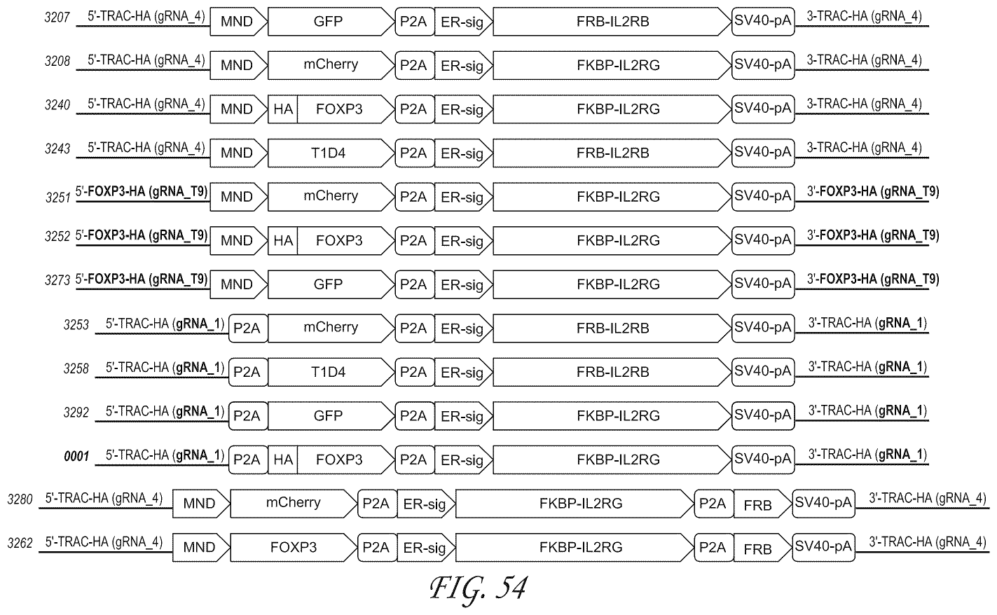

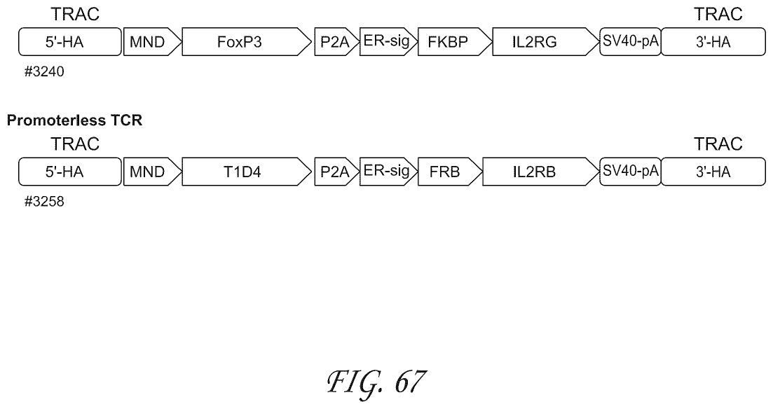

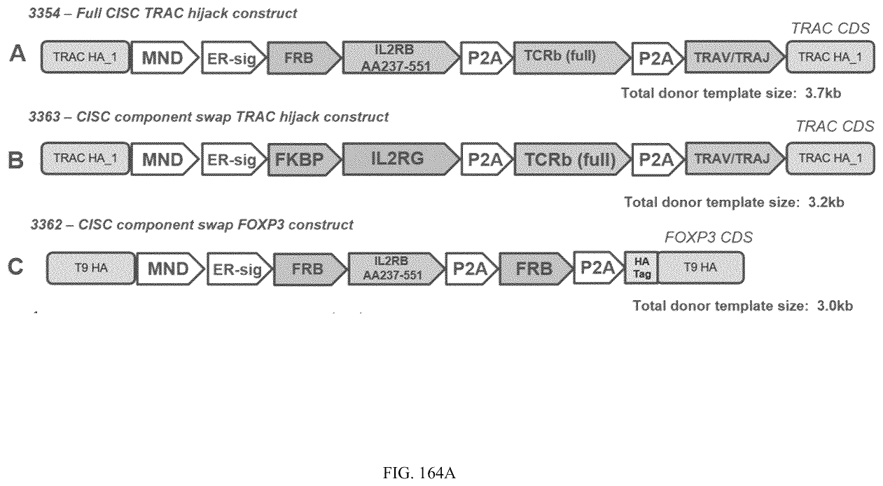

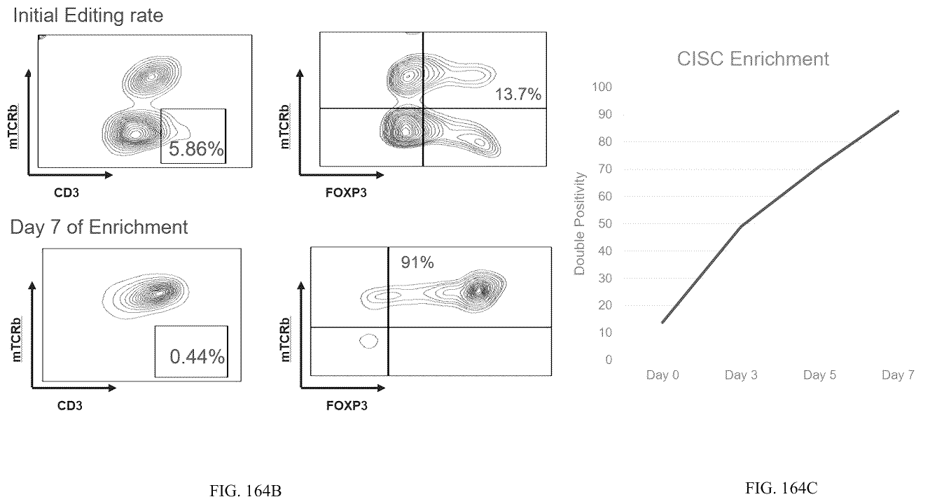

Provided herein are different methods of editing the TRAC locus in a cell. In some embodiments, the TRAC locus is edited to introduce a nucleic acid encoding an antigen-specific receptor, such as an exogenous TCR or CAR, and a nucleic acid encoding a first or second CISC component as described herein. In some embodiments, a promoter capture method is used in which a native/naturally-occurring/endogenous promoter is relied upon for control of inserted TCR-encoding nucleic acid, which may be followed by nucleic acid encoding one or more CISC components as described herein (see e.g., FIGS. 54, 68, and 70). In some embodiments such an endogenous promoter capture method comprises in-frame knock-in to TRAC exon 1 to drive expression of a TCR relying on endogenous TRAC promoter. In some embodiments, a TCR knock-in strategy is employed and comprises knock-in of a promoter and TCR-encoding nucleic acid, which may be followed by nucleic acid encoding one or more CISC components as described herein (see e.g., FIG. 67). It should be understood that any embodiments of a method comprising insertion of a TCR or nucleic acid encoding a TCR can be modified to insert a CAR or a nucleic acid encoding a CAR. In some embodiments, the native/naturally-occurring/endogenous TCR gene or fragments thereof are hijacked by insertion of a promoter upstream from the native/naturally-occurring/endogenous and optionally upstream from nucleic acid encoding one or more CISC components as described herein (see e.g., FIG. 164).

### Promoter Capture Methods

In some embodiments, a TRAC locus in a cell is edited by promoter capture (e.g., the method depicted in FIGS. 54, 68, and 70). In some embodiments of the methods provided herein, a cell is edited by inserting a nucleic acid molecule comprising a nucleic acid encoding an exogenous TCR or CAR into the TRAC locus, wherein the nucleic acid encoding the TCR or CAR is inserted downstream of (e.g., 10 to 10,000 bp downstream from) the endogenous TRAC promoter and in-frame with the TRAC coding sequence, such that the endogenous TRAC promoter becomes operably linked to the inserted nucleic acid and drives expression of the exogenous TCR or CAR. Thus, in some embodiments, the exogenous TCR or CAR is expressed at a similar level to expression of the endogenous TCR before the genetic modification. In some embodiments, the nucleic acid molecule comprising a nucleic acid encoding the exogenous TCR or CAR further comprises a nucleic acid encoding a first or second CISC component as described herein. In some embodiments, the nucleic acid molecule comprising a nucleic acid encoding the exogenous TCR or CAR further comprises a nucleic acid encoding a third CISC component as described herein.

### TCR/CAR Knock-In Methods

In some embodiments, a TRAC locus in a cell is edited by knocking in a full TCR (e.g., an islet TCR) or CAR with a promoter (e.g., MND promoter) operably linked to the polynucleotide/nucleic acid encoding the TCR or CAR and terminating in a stop codon and polyadenylation signal for expression independent of the endogenous TRAC regulatory elements. See FIG. 67 as an example of such a method. In some embodiments, a cell is edited by inserting a nucleic acid molecule comprising a heterologous promoter operably linked to a polynucleotide/nucleic acid encoding a TCR or CAR into the TRAC locus, such that the heterologous promoter drives expression of the inserted TCR or CAR while simultaneously disrupting the endogenous TRAC gene. In some embodiments, the nucleic acid molecule comprising a polynucleotide/nucleic acid encoding the exogenous TCR or CAR further comprises a polynucleotide/nucleic acid encoding a first or second CISC component as described herein. In some embodiments, the polynucleotide molecule comprising a nucleic acid encoding the exogenous TCR or CAR further comprises a nucleic acid encoding a third CISC component as described herein.

### Hijacking the TRAC Gene with a Promoter

In some embodiments, a TRAC locus in a cell is edited by hijacking the TRAC gene with a promoter (e.g., MND promoter) as shown in FIG. 164. In some embodiments, a cell is edited by inserting a polynucleotide molecule comprising a promoter operably linked to (a) a nucleic acid encoding a full-length TCRβ protein, and to a nucleic acid encoding TCRα variable (TRAV) and TCR joining (TRAJ) regions, where the coding sequences of the TRAV and TRAJ regions are inserted in-frame with the coding sequences encoding the TCRα constant regions, such that the inserted heterologous promoter controls transcription of a heterologous TCRβ protein and transcription of a TCRα protein comprising heterologous TRAV/TRAJ amino acid sequences and an endogenous TCRα constant region amino acid sequence. This embodiment utilizes the endogenous 3′ regulatory region from the endogenous TRAC gene. In some embodiments, the polynucleotide molecule comprising a nucleic acid encoding the exogenous TCR or CAR further comprises a nucleic acid encoding a first or second CISC component as described herein. In some embodiments, the polypeptide molecule comprising a nucleic acid encoding the exogenous TCR or CAR further comprises a nucleic acid encoding a third CISC component as described herein.

Provided herein are airT cells, e.g., cells that are edited in one, two, or more than two loci by the insertion of nucleic acids encoding at least two CISC components (e.g., at least a first and a second CISC component) and optionally a third CISC component.

Some embodiments include use of such systems to edit a FOXP3 locus gene and TRAC locus in a Treg cell. More embodiments relate to use of gene-edited Treg cells to suppress activation and/or proliferation of certain populations of cells. Such suppression can be performed either in vitro, ex vivo, or in vivo (e.g., but administering the cells to a subject with or without a ligand (such as rapamycin) to activate or maintain activation of the cells' suppressive phenotype).

Some embodiments of the methods and compositions provided herein relate to artificial antigen-specific immunoregulatory T (airT) cells. AirT cells may also be referred to as “edTreg” or “Edited Treg” cells or “EngTreg” or “Engineered Treg” cells. An AirT cell can also be a Tr1 cell, e.g., a cell that expresses IL-10. Some embodiments include an artificially engineered T cell (e.g., a T lymphocyte). Some embodiments include a CD4+CD25+ T cell having an artificial modification of a forkhead box protein 3/winged helix transcription factor (FOXP3) gene, and that constitutively expresses a FOXP3 gene product at a FOXP3 expression level that is equal to or greater than the FOXP3 expression level of a naturally occurring regulatory T (Treg) cell; and at least one transduced polynucleotide encoding an antigen-specific T cell receptor (TCR) polypeptide.

Some embodiments provided herein relate to efficient editing of more than one genetic locus in a cell using a chemical-inducible signaling complex (CISC) system in which components of the CISC system are provided with increased levels of expression compared to a CISC system with an alternative orientation and/or inclusion of elements. In some embodiments, a gene-editing system, as described herein, comprising one or more (e.g., two) constructs, each construct comprising a promoter and one or more elements (e.g., a polynucleotide/nucleic acid encoding a first CISC component, and a polynucleotide/nucleic acid encoding a TCR) that are configured to provide optimal expression of all CISC components. This is based, at least in part, on the finding that orientation of the split CISC (comprising first and second CISC components on different cassettes, each cassette targeting a different gene locus) is critical to their function. It was unexpectedly found that split CISC function is dependent upon expression of the different components of the CISC system and/or location of these elements within a construct/cassette (e.g., an HDR cassette). Accordingly, in some embodiments, one or more constructs for a gene-editing system as described herein comprises a promoter (e.g., an MND promoter) that results in a high expression of CISC component compared to promoters (e.g., EF1-alpha) that results in a lower expression of CISC components. In some embodiments, each of the constructs targets a different gene locus.

### Methods of Suppressing Proliferation of Teff Cells Using Genetically Modified/Engineered airT Cells; and Bystander Effect

Some embodiments provided herein relate to methods of suppressing proliferation of Teff cells using genetically modified/engineered Treg cells. Some such embodiments can include a bystander effect in which the Treg cell comprises a TCR specific for a first epitope of an antigen, and the Teff cells comprise a TCR specific for a second epitope of the antigen.

Certain features useful in certain embodiments provided herein are disclosed in Int. App. No. PCT/US2020/039445 filed Jun. 24, 2020 entitled “ARTIFICIAL ANTIGEN-SPECIFIC IMMUNOREGULATORY T (AIRT) CELLS”; U.S. Prov. App. No. 62/987,810 filed Mar. 10, 2020 entitled “ARTIFICIAL ANTIGEN-SPECIFIC IMMUNOREGULATORY T (AIRT) CELLS”; and U.S. Prov. App. No. 62/867,670 filed Jun. 27, 2019 entitled “ANTIGEN-SPECIFIC TREG THERAPY FOR AUTOIMMUNE DISEASE” which are each expressly incorporated by reference in its entirety. Certain other features useful in certain embodiments provided herein are disclosed in Int. App. No. PCT/US2017/065746 filed Dec. 12, 2017 entitled “METHODS OF EXOGENOUS DRUG ACTIVATION OF CHEMICAL-INDUCED SIGNALING COMPLEXES EXPRESSED IN ENGINEERED CELLS IN VITRO AND IN VIVO”; and Int. App. No. PCT/US2019/029118 filed Apr. 25, 2019 entitled “RAPAMYCIN RESISTANT CELLS” which are each expressly incorporated by reference in their entirety.

In some embodiments, the airT cells are capable of mediating antigen-specific immunosuppression when induced by a specific antigen that is recognized by a TCR, such as an autoantigen, an allergen, or another antigen associated with the pathogenesis of an inflammatory condition characterized by an excessive immune response. Significantly, production of the present airT cells does not require the time, costs, and inefficiencies associated with isolation of relatively rare (14% of human PBMC) natural Treg cells as a starting material for gene editing, thus affording certain advantages for the generation of therapeutically effective amounts of desired cells for adoptive immunotherapy. The airT cells of the present application can be formed from use of cells that are not T cells, e.g., a stem cell. In some embodiments, an airT cell is produced by gene editing of an induced pluripotent stem cell (iPSC). In some embodiments, an airT cell is produced by gene editing of a CD34+ hematopoietic stem cell (HSC). In some embodiments, a stem cell is first differentiated into a T cell and then that T cell is transformed into an airT cell.

In some embodiments, the airT cell expresses a functional TCR that specifically recognizes an antigen associated with pathogenesis of an autoimmune condition, an allergic condition, an inflammatory condition, or solid organ transplant or graft-versus-host disease, such as a TCR comprising any of the TCR polypeptide sequences disclosed herein or any of the TCR polypeptides encoded by the TCR-encoding nucleic acid sequences disclosed herein, including those set forth in the Drawings.

In some embodiments, the airT cell expresses a functional TCR that specifically recognizes an antigen associated with pathogenesis of an autoimmune condition, an allergic condition, or an inflammatory condition, such as any of the polypeptide autoantigens, allergens, and/or inflammation-associated antigens comprising the polypeptide antigen amino acid sequences disclosed herein, or any polypeptide antigens that are immunologically cross-reactive with the polypeptide autoantigens, allergens, and/or inflammation-associated antigens comprising the polypeptide antigen amino acid sequences disclosed herein, including those set forth in the Drawings.

Certain of the herein disclosed embodiments relate to gene editing strategies for the generation of the airT cells that include surprisingly advantageous functional linkage of (i) stable FoxP3 expression that results from targeted FoxP3 gene editing, including the introduction of a promoter, such as a constitutive promoter, to drive FoxP3 expression in cells that did not previously express FoxP3, wherein the FoxP3 expression is at a level equal to or greater than the FoxP3 expression level of a naturally occurring regulatory T (Treg) cell, to maintain a stable FoxP3-controlled immunoregulatory (immunosuppressive) program of the airT cell, and (ii) stable expression of an exogenously sourced TCR in the same cells by gene editing to introduce into the airT cell the particular presently disclosed nucleotide sequences encoding TCR that recognize antigens associated with pathogenesis of an autoimmune condition, an allergic condition, or an inflammatory condition, to permit selection and expansion of engineered T cells characterized by stable immunosuppressive potential that co-segregates with desired TCR expression. Without wishing to be bound by theory, it is believed that by artificially engineered stable FoxP3 expression, some embodiments of the presently described airT cells include safe and effective adoptive transfer immunotherapy cells for indications where antigen-specific immunosuppression is desirable, such as autoimmune, allergic, graft vs host disease (GVHD), or other inflammatory conditions, without the risks associated with natural Treg plasticity (e.g., reversion to T effector behavior).

Production of the presently disclosed airT cell advantageously and in some embodiments does not include first isolating natural Treg cells, which as noted above, are naturally present in peripheral blood at a low frequency, representing only about 14% of human peripheral blood mononuclear cells. Instead, as described herein, generation of airT cells can be achieved by isolating T cells, such as CD4+ T cells or CD8+ T cells, which although heterogeneous with respect to other cell surface markers may comprise approximately 25-60% of human PBMC and thus represent a relatively abundant starting material for gene editing according to the various strategies provided herein.

The present antigen-specific immunoregulatory T (airT) cell compositions and methods will, in certain embodiments, find uses in the treatment and/or amelioration of certain autoimmune conditions, allergic conditions, and/or inflammatory conditions, including in adoptively transferable immunotherapy, where stable airT cell viability and maintenance of antigen-specific immunoregulatory function provide unprecedented benefits.

In certain embodiments the airT cell described herein is unexpectedly capable of inducing an antigen-specific immunosuppressive response when stimulated by an antigen associated with pathogenesis of an autoimmune condition, an allergic condition, or an inflammatory condition such as one of the antigens disclosed herein. Such antigen-specifically induced immunosuppression may comprise one or more of: (i) inhibition of either or both of activation and proliferation of effector T cells that recognize the antigen that is specifically recognized by the airT TCR comprising the TCR polypeptide that is encoded by the at least one transduced polynucleotide/nucleic acid, (ii) inhibition of expression of inflammatory cytokines or inflammatory mediators by effector T cells that recognize the antigen that is specifically recognized by the airT TCR comprising the TCR polypeptide that is encoded by the at least one transduced polynucleotide/nucleic acid (iii) elaboration of one or more immunosuppressive cytokines or anti-inflammatory products, for example, elaboration of one or more inhibitory mechanisms including release of immunosuppressive cytokines or perforin/granzyme, induction of indoleamine 2,3-dioxygenase (IDO), competition for IL2 or adenosine, catabolism of tryptophan, and expression of inhibitory receptors by the airT cell, by the airT cell, (iv) inhibition of either of or both activation and proliferation of effector T cells that do not recognize the antigen that is specifically recognized by the airT TCR comprising the TCR polypeptide that is encoded by the at least one transduced polynucleotide/nucleic acid, and (v) inhibition of expression of inflammatory cytokines or inflammatory mediators by effector T cells that do not recognize the antigen that is specifically recognized by the airT TCR comprising the TCR polypeptide that is encoded by the at least one transduced polynucleotide/nucleic acid, or (vi). In some embodiments such antigenic stimulation of the airT cell is HLA-restricted.

In some embodiments, the generation of the present airT cells, which stably express FoxP3 as described herein, overcomes certain disadvantages associated with prior methodologies in which FOXP3 transgene expression was achieved by retroviral or lentiviral gene transfer. The resulting virally FoxP3-transduced cell populations were genetically heterogeneous by virtue of having randomly integrated FOXP3 transgenes of varying stability and varying expression levels at various genomic sites. Despite at least transiently exhibiting Treg characteristics such as phenotypic markers and cytokine expression profile, such transduced populations were also potentially compromised by carrying a concomitant risk of genotoxicity, as well as vulnerability to silencing by local regulatory elements at sites of viral integration.

To avoid these risks, some embodiments provided herein include the use of specifically targeted gene editing for artificial modification of the FOXP3 gene instead of relying on viral FOXP3 gene transfer and, optionally specifically targeted TCR gene editing. Certain embodiments described herein utilize lentiviral gene delivery to introduce candidate autoimmune-related TCRs (or CARs) into T cells, such as CD4 T cells or CD8 T cells, followed by FOXP3 gene editing of the cells to force stable FoxP3 expression. In some related embodiments, this approach is combined with any one of the TRAC locus editing methods disclosed herein, e.g., gene editing methods to simultaneously delete the endogenous TCR gene (e.g., via inactivation, also referred to herein as “knockout”).

As an alternative strategy distinct from lentiviral TCR delivery, some embodiments described herein relate to simultaneous gene editing at different alleles of the same gene locus e.g., single-locus bi-allelic dual editing in which dual-editing is achieved at a single locus (e.g., with a single guide RNA and AAV donor homology constructs). In some embodiments, methods of gene editing at two alleles of the same locus comprise i) inserting a first donor template into a first allele of a locus; and ii) inserting a second donor template into a second allele of the same locus. Each donor template may be inserted into an allele of the locus by any of the gene editing methods provided herein. In some embodiments, the method comprises providing an RNA-guided nuclease or a nucleic acid encoding the RNA-guided endonuclease, and a guide RNA that directs the RNA-guided nuclease to cleave a nucleotide sequence within the locus. Thus, an RNA-guided nuclease cleaves nucleotide sequences within both alleles of the locus and each donor template is inserted into a separate allele of the locus by homology-directed repair. Competition for the single double-strand break by both donor templates results in a predicable subpopulation of bi-allelic edited cells that have incorporated one copy of each donor template. In some embodiments, methods of gene editing at two alleles of the same locus comprise providing to the cell a nuclease (e.g., meganuclease, TALEN, or ZFN) that cleaves a nucleotide sequence within both alleles of the locus. Mono-allelic vs bi-allelic modification may comprise using a locus and HDR templates that have a very high HDR rate (such as the TRAC locus in primary T-Cells), and choosing an endonuclease with a lower or higher efficiency. Alternatively gRNAs are selected so that it is more efficient on one allele. Any method of assessing single or bi-allelic modifications can be used, e.g., sequencing analysis.

As another alternative, some embodiments described herein relate to simultaneous gene editing at two different gene loci, e.g., two-loci dual editing in which a distinct gene editing event takes place at each of two loci (e.g., with two different guide RNAs and locus-specific AAV donor homology cassettes). In some embodiments, methods of gene editing at two distinct loci comprise i) inserting a first donor template into a first locus; and ii) inserting a second donor template into a second locus that is different from the first locus. Each donor template may be inserted into a respective locus by any of the gene editing methods provided herein. In some embodiments, the method comprises providing an RNA-guided nuclease or a nucleic acid encoding the RNA-guided endonuclease, a first guide RNA that directs the RNA-guided nuclease to cleave a nucleotide sequence within the first locus, and a second guide RNA that directs the RNA-guided nuclease to cleave a nucleotide sequence within the second locus. Thus, an RNA-guided nuclease cleaves both loci after being directed to each locus by respective guide RNAs, and each donor template is inserted by homology-directed repair. In some embodiments, methods of gene editing at two distinct loci comprise providing to the cell a first nuclease (e.g., meganuclease, TALEN, or ZFN) that cleaves a nucleotide sequence within a first locus; and a second nuclease (e.g., meganuclease, TALEN, or ZFN) that cleaves a nucleotide sequence within a second locus. Inserting a first donor template and second donor template into a first and second locus, respectively, can be done by contacting a cell with (i) a first polypeptide comprising a nucleic acid encoding a first CISC component comprising a first extracellular binding domain, first transmembrane domain, and first intracellular signaling domain (ii) a second polypeptide comprising a second nucleic acid encoding a second CISC component comprising a second extracellular binding domain, a transmembrane domain, and a second intracellular signaling domain, (iii) a first endonuclease, or nucleic acid encoding the first endonuclease, that can cleave a first nucleotide sequence within a first locus, (iv) a second endonuclease, or nucleic acid encoding the second endonuclease, that can cleave a second nucleotide sequence within a second locus, such that the first polynucleotide or fragment thereof is incorporated into the first locus, and the second polynucleotide or fragment there is inserted in the second locus; as described above.

By these approaches, engineered FOXP3 and TCR genes (or any other payload) may be delivered to a single specific gene locus or to two different specific loci. As also described herein, in some embodiments this strategy may further include incorporating split chemical-induced signaling complex (split CISC) components that permit selective expansion of only those T cells that express both the FOXP3 and the inserted TCR in the same cell, thereby enriching for airT cells. Provided herein is a method of selective expansion of cells, the method comprising (i) inserting into one or more cells in a population (a) a first polynucleotide molecule comprising a nucleic acid encoding a first CISC component; and (b) a second polynucleotide molecule comprising a nucleic acid encoding a second CISC component, wherein the first and second CISC components are capable of specifically binding to a CISC inducer molecule and dimerizing in the CISC inducer molecule, and (ii) contacting the cell with the CISC inducer molecule to promote proliferation and/or activation of cells expressing both the first and second CISC components. In some embodiments, the first or second polynucleotide molecule comprises a nucleic acid encoding a third CISC component that is soluble, cytosolic, and capable of specifically binding to the CISC inducer molecule, where expression of the third CISC component prevents or reduces an effect of the CISC inducer molecule on endogenous signaling pathways. Any transfection and genome modification methods may be used for inserting a first and second nucleic acid or nucleic acid molecule. In some embodiments, inserting a first donor template and second donor template into a first and second locus can be done by contacting a cell with (i) a first nucleic acid comprising a nucleic acid encoding a first CISC component comprising a first extracellular binding domain, first transmembrane domain, and first intracellular signaling domain (ii) a second nucleic acid comprising a second nucleic acid encoding a second CISC component comprising a second extracellular binding domain, a transmembrane domain, and a second intracellular signaling domain, (iii) a first endonuclease, or nucleic acid encoding the first endonuclease, that can cleave a first nucleotide sequence within a first locus, (iv) a second endonuclease, or nucleic acid encoding the second endonuclease, that can cleave a second nucleotide sequence within a second locus, such that the first polynucleotide or fragment thereof is incorporated into the first locus, and the second polynucleotide or fragment there is is inserted in the second locus; as described above.

### Chemical-Induced Signaling Complex (CISC)

As described herein, some embodiments exploit a split chemical-induced signaling complex (split CISC) strategy by which gene-edited airT cells may be generated and selectively expanded on the basis of successful expression in the same cells of both (i) a constitutively expressed FoxP3 gene-edited gene product, the expression of which is associated with cell surface expression of a first CISC component that specifically binds to a CISC inducer molecule, the first CISC component being present as a transmembrane fusion protein having a first extracellular CISC inducer molecule binding domain, a transmembrane domain, and a first intracellular activation signal transduction domain; and (ii) a transduced heterologous TCR gene-edited gene product, the expression of which is associated with cell surface expression of a second CISC component that is different than the first CISC component and specifically binds to the CISC inducer molecule, the second CISC component being present as a transmembrane fusion protein having a second extracellular CISC inducer molecule binding domain, a transmembrane domain, and a second intracellular activation signal transduction domain that is different than the first intracellular activation signal transduction domain. In some embodiments, a first CISC component comprises an FKBP extracellular domain and an IL2RB intracellular domain, and a second CISC component comprises an FRB extracellular domain and an IL2RG intracellular domain. In some embodiments, a first CISC component comprises an FKBP extracellular domain and an IL2RG intracellular domain, and a second CISC component comprises an FRB extracellular domain and an IL2RB intracellular domain. In some embodiments, one or both of the first and second CISC components comprises a hinge domain positioned between the extracellular domain and the transmembrane domain. In some embodiments, each of the first and second CISC components comprise a hinge domain positioned between the extracellular domain and the transmembrane domain.

In certain embodiments, CD3+ T cells, CD4+ T cells or CD8+ T cells are enriched from a biological sample such as peripheral blood mononuclear cells (PBMC) prior to gene editing (e.g., dual editing) as described herein. In certain embodiments enriched CD3+, CD4+ T cells, or CD8+ T cells are non-specifically activated (e.g., with solid-phase immobilized anti-CD3 and anti-CD28 antibodies) prior to gene editing (e.g., dual editing) as described herein.

In some embodiments, exposure of dual-edited T cells as described herein to the CISC inducer molecule results in binding of the inducer molecule to the extracellular domains of both the first and second CISC components and heterodimer formation by the first and second CISC components to activate a functional signal transduction complex that is formed by the first and second intracellular activation signal transduction domains. As a consequence, the population of airT cells in which are expressed both the first and second CISC components, and hence, payloads with the first and second CISC, e.g., both FoxP3 and the heterologous TCR, is selectively expanded in culture conditions where the CISC inducer molecule is supplied in replacement of the endogenous signal transducer.

In some embodiments, the two gene editing events that give rise to expression in the present airT cells, of the first CISC component concomitant with the FoxP3 gene product and of the second CISC component concomitant with the TCR gene product, may be designed to take place in different alleles of the same gene locus (e.g., bi-allelic dual editing), or at two different gene loci (e.g., two-loci dual editing). In some embodiments, methods of gene editing at two alleles of the same locus comprise i) inserting a first donor template into a first allele of a locus; and ii) inserting a second donor template into a second allele of the same locus. Each donor template may be inserted into an allele of the locus by any of the gene editing methods provided herein. In some embodiments, the method comprises providing an RNA-guided nuclease or a nucleic acid encoding the RNA-guided endonuclease, and a guide RNA that directs the RNA-guided nuclease to cleave a nucleotide sequence within the locus. Thus, an RNA-guided nuclease cleaves nucleotide sequences within both alleles of the locus and each donor template is inserted into a separate allele of the locus by homology-directed repair. In some embodiments, methods of gene editing at two alleles of the same locus comprise providing to the cell a nuclease (e.g., meganuclease, TALEN, or ZFN) that cleaves a nucleotide sequence within both alleles of the locus.

In some embodiments, methods of gene editing at two distinct loci comprise i) inserting a first donor template into a first locus; and ii) inserting a second donor template into a second locus that is different from the first locus. Each donor template may be inserted into a respective locus by any of the gene editing methods provided herein. In some embodiments, the method comprises providing an RNA-guided nuclease or a nucleic acid encoding the RNA-guided endonuclease, a first guide RNA that directs the RNA-guided nuclease to cleave a nucleotide sequence within the first locus, and a second guide RNA that directs the RNA-guided nuclease to cleave a nucleotide sequence within the second locus. Thus, an RNA-guided nuclease cleaves both loci after being directed to each locus by respective guide RNAs, and each donor template is inserted by homology-directed repair. In some embodiments, methods of gene editing at two distinct loci comprise providing to the cell a first nuclease (e.g., meganuclease, TALEN, or ZFN) that cleaves a nucleotide sequence within a first locus; and a second nuclease (e.g., meganuclease, TALEN, or ZFN) that cleaves a nucleotide sequence within a second locus.

In some embodiments, a locus comprises a nucleic acid sequence encoding a gene. In some embodiments, a locus comprises a nucleic acid comprising one or more exons encoding a gene, and one or more nucleotides between the one or more exons. In some embodiments, a locus comprises a nucleic acid comprising a one or more regulatory elements and a nucleic acid sequence encoding a gene. In some embodiments, a single gene in a genome comprises multiple loci. In some embodiments, multiple loci comprise multiple nucleic acid sequences that do not share common nucleotides. In some embodiments, multiple loci comprise multiple nucleic acid sequences, and no nucleotide belongs to more than one locus. In some embodiments, a third CISC component that specifically binds to the CISC inducer molecule may also be co-expressed with either the FoxP3 gene product or the TCR gene product. The third CISC component remains at an intracellular locale when expressed and acts as a decoy to bind and thereby avoid toxicities associated with certain CISC inducer molecules that may reach the cell interior.

Details of CISC systems, including structures of first, second and third CISC components and of CISC inducer molecules are described elsewhere herein and in WO/2018/111834 and WO/2019/210078, which are both expressly incorporated by reference in their entireties. Briefly, WO/2018/111834 describes compositions and methods for genetically editing host cells by knock-in (insertion) of genetic constructs encoding a ligand-dimerizable fusion protein chemical-induced signaling complex (CISC). Cellular expression of both fusion protein subunits followed by exposure of the host cells to the chemical ligand permits ligand-induced dimerization of the CISC to transduce a cellular activation signal. The CISC system thus provides selection and expansion (e.g., activation-induced proliferation) of cells that have undergone gene modification to incorporate both of the CISC components, to select cells in which gene editing has occurred. WO/2019/210078 describes gene editing compositions and methods in which nucleic acid sequences encoding first and second CISC subunit components are introduced to host cells as part of gene editing at a single targeted FOXP3, TRAC, or AAVS1 gene locus. Chemical ligand-induced dimerization of the CISC can induce a biological signal transduction event for selection and expansion of edited cells. Optionally and in some related embodiments a nucleic acid encoding a third CISC subunit component is also expressed in the host cells; the third CISC component remains intracellularly expressed as a decoy to decrease potential harmful effects on the cell of internalized CISC ligand.

Exemplary first and second CISC subunit components may comprise functional intracellular signal transduction domains of IL2-receptor beta and gamma subunits (IL2RB, IL2RG). An exemplary third CISC component may comprise a functional rapamycin-binding domain of FK506-binding protein (FKBP). An exemplary third CISC component may comprise an FKBP-rapamycin-binding protein (FRB), which binds the complex of FKBP-bound rapamycin.

### Polynucleotides Comprising Nucleic Acids Encoding CISC Components

Some embodiments of the methods provided herein include inserting into the genome of a cell (i) a first polynucleotide molecule comprising a first nucleic acid encoding a first CISC component, and (ii) a second polynucleotide molecule comprising a second nucleic acid encoding a second CISC component encoding a second CISC component. In some embodiments, the first polynucleotide molecule comprises a nucleic acid encoding a first CISC component comprising an FKBP extracellular domain, a transmembrane domain, and an IL2RB intracellular signaling domain, and the second polynucleotide molecule comprises a second nucleic acid encoding a second CISC component comprising an FRB extracellular domain, a transmembrane domain, and an IL2RG intracellular signaling domain. In some embodiments, the first polynucleotide molecule comprises a nucleic acid encoding a first CISC component comprising an FKBP extracellular domain, a transmembrane domain, and an IL2RG intracellular signaling domain, and the second polynucleotide molecule comprises a second nucleic acid encoding a second CISC component comprising an FRB extracellular domain, a transmembrane domain, and an IL2RB intracellular signaling domain. In some embodiments, the nucleic acids encoding the first or second CISC component further comprises a nucleic acid encoding a third CISC component comprising a soluble FRB domain, and the third CISC component does not comprise a transmembrane or extracellular domain, such that the third CISC component is localized to the cytoplasm of the cell when expressed. In some embodiments, the first polynucleotide molecule encodes the first CISC component and the third CISC component in one open reading frame, where the nucleotide sequences encoding the first and third CISC components are separated by a nucleotide sequence encoding a self-cleaving peptide. In other embodiments, the second polynucleotide molecule encodes the second CISC component and the third CISC component in one open reading frame, where the nucleic acids encoding the second and third CISC components are separated by a nucleotide sequence encoding a self-cleaving peptide. In some embodiments, the self-cleaving peptide is selected from F2A, P2A, T2A, E2A, and combinations thereof (see, e.g., WO 2017/127750). This family of self-cleaving peptides, referred to as 2A peptides, has been described in the art (see, e.g., Kim, J. H. et al. PLoS ONE 2011;6:e18556).

### FOXP3/airT Phenotypic Markers and Suppressor Function

FOXP3 gene editing may include artificial modification of a native FOXP3 gene locus and/or may also include artificial modification of a chromosomal site other than a native FOXP3 gene locus. For example, gene editing may include knock-in (e.g., insertion) of a nucleic acid molecule comprising an exogenous FOXP3-encoding nucleic acid operably linked to a constitutive promoter at a chromosomal site other than a native FOXP3 gene locus, such as a T cell receptor alpha chain (TRAC) gene locus, a T cell receptor beta chain (TCRB) locus or an adeno-associated virus integration site 1 (AAVS1) or another gene locus. Certain embodiments thus surprisingly provide the herein described airT cells, which are capable of mediating antigen-specific immunosuppression, when artificial FoxP3 gene sequences are introduced to a genomic site other than the native FOXP3 gene locus (e.g., in the TRAC locus) and are able constitutively to express a FOXP3 gene product at a level that is equal to or greater than the FOXP3 expression level of a naturally occurring Treg cell. In some embodiments of the methods provided herein, a heterologous promoter (e.g., an MND promoter) is inserted upstream of the first coding exon of FOXP3, and downstream of (e.g., 10 to 10,000 bp downstream from) one or more regulatory elements (e.g., a TSDR) of the endogenous FOXP3 coding sequence, such that the inserted promoter drives transcription of the endogenous FOXP3 gene independently of the upstream regulatory elements. In some embodiments, the method comprises inserting an MND promoter downstream of (e.g., 10 to 10,000 bp downstream from) a TSDR of the FOXP3 gene and upstream of the first coding exon of the FOXP3 gene.

The two (or more) gene editing events that give rise in certain embodiments to expression in the present airT cells, of the FoxP3 gene product (or another gene that regulates suppressive function of the cell) concomitant with the first CISC component and of the TCR gene product concomitant with the second CISC component concomitant, may be designed to take place in different alleles of the same gene locus (e.g., bi-allelic dual editing), or at two different gene loci (e.g., two-loci dual editing). In some embodiments, a locus comprises a nucleic acid sequence encoding a gene. In some embodiments, a locus comprises a nucleic acid comprising one or more exons encoding a gene, and one or more nucleotides between the one or more exons. In some embodiments, a locus comprises a nucleic acid comprising a one or more regulatory elements and a nucleic acid sequence encoding a gene. In some embodiments, a single gene in a genome comprises multiple loci. In some embodiments, multiple loci comprise multiple nucleic acid sequences that do not share common nucleotides. In some embodiments, multiple loci comprise multiple nucleic acid sequences, and no nucleotide belongs to more than one locus. In some embodiments the presently disclosed airT cell is surprisingly capable of expressing the FOXP3 gene product at an expression level sufficient for the airT cell to maintain a CD4+CD25+ phenotype for at least 21 days in vitro, or for at least 60 days in vivo following adoptive transfer to an immunocompatible mammalian host in need of antigen-specific immunosuppression, while functionally expressing a herein-disclosed TCR that specifically recognizes an antigen associated with pathogenesis of an autoimmune condition, an allergic condition, or an inflammatory condition, or a TCR that specifically recognizes a herein-disclosed antigen associated with pathogenesis of an autoimmune condition, an allergic condition, or an inflammatory condition.

The CD4+CD25+ or CD8+ airT cell disclosed herein thus in certain embodiments relates to a genetically engineered cell obtained by artificial modification of a FOXP3 gene in a CD4+CD25⁻ T cell. in some embodiments, the artificial modification causes the airT cell to constitutively express a FOXP3 gene product at a FOXP3 expression level that is equal to or greater than the FOXP3 expression level of a naturally occurring regulatory T (Treg) cell. In some embodiments of the methods provided herein, a heterologous promoter (e.g., an MND promoter) is inserted upstream of the first coding exon of FOXP3, and downstream of (e.g., 10 to 10,000 bp downstream from) one or more regulatory elements (e.g., a TSDR) of the endogenous FOXP3 coding sequence, such that the inserted promoter drives transcription of the endogenous FOXP3 gene independently of the upstream regulatory elements. In some embodiments, the method comprises inserting an MND promoter downstream of (e.g., 10 to 10,000 bp downstream from) a TSDR of the FOXP3 gene and upstream of the first coding exon of the FOXP3 gene. In some embodiments, the airT cell may also express the CD25, CD152, and/or ICOS cell surface markers at levels which are characteristic of immunoregulatory cells such as natural Treg. Unlike natural Treg, however, in some embodiments the present airT cells may exhibit a HeliosLo cell surface phenotype, e.g., an expression level of the Helios cell surface marker that is decreased, in a statistically significant manner, relative to the Helios expression level in naturally occurring Treg cells. It is to be understood that methods described herein that comprise manipulation of CD4+ cells or editing of the genome of CD4+ cells, can be applied to other types of cells (e.g., CD8+ cells).

Exemplary details of gene editing strategies to induce FoxP3 expression in T cells are described herein and in WO/2018/080541 and in WO/2019/210078, which are expressly incorporated by reference in their entireties. Exemplary details of forced FOXP3 expression by gene editing including knock-in (insertion) of a full length, codon-optimized FoxP3 cDNA into the FOXP3 or AAVS1 locus may be found in WO/2019/210042, which is expressly incorporated by reference in its entirety. In some embodiments, only a promoter (e.g., a constitutively active promoter) is inserted in the FOXP3 locus without inserting any polypeptide-encoding nucleic acid.

Briefly, WO/2018/080541 describes CD4+ T cells in which stable expression of endogenous FoxP3 is engineered by gene editing using Cas9, ZFN, or TALEN to knock-in (e.g., by insertion) a constitutive promoter that is an EF1a, PGK, or MND promoter.

Any method for gene editing (or genome editing) is contemplated herein, including, without limitation, viral and non-viral approaches that enable targeted gene-editing (e.g., Cas9, ZFN, TALEN). Accordingly, a method of gene editing as provided herein may make use of a nuclease to target a locus or a targeted locus on a nucleic acid sequence. In some embodiments, a nuclease is Cas9, a zinc-finger nuclease or TALEN. FoxP3 expression may be achieved by targeted knock-in (insertion), at the FOXP3 gene locus, of a polynucleotide comprising a regulatory sequence operably linked to a coding sequence for the first expressed FOXP3 exon. The regulatory sequence may comprise a promoter which in some embodiments may be the MND, PGK, or EF1a promoter, or another inducible, weak, or constitutive promoter. Exemplary edited FOXP3+ cells may comprise a fully methylated FOXP3 gene intronic regulatory T cell-specific demethylation region (TSDR) upstream of the knocked-in promoter integration site.

WO/2019/210078 describes forced expression of FoxP3 in CD4+ T cells to achieve cells having a Treg-like phenotype, methods of selecting for such cells to obtain a Treg-enriched preparation, and methods for expanding populations of such cells in vitro. WO/2019/210078 also describes compositions and methods for targeted gene editing at the FOXP3, AAVS1, and/or TCRalpha (TRAC) loci, including guide RNA (gRNA) sequences specific for each of these loci and donor templates for gene editing by HDR. WO/2019/210078 also describes a CISC system in which a chemical-ligand dimerization of first and second CISC components results in an activation signal that effects T cell proliferation and hence selective expansion of edited T cells. WO/2019/210078 describes first and second CISC components in which the CISC inducer molecule is rapamycin or any of a large number of disclosed rapamycin analogues, derivatives, and mimetics, and in which the activation signal transduction domains of the CISC components comprise functional portions of the cytoplasmic domains of the IL-2 receptor beta (IL2Rb, also referred to as IL2Rβ) and IL-2 receptor gamma (IL2Rg, also referred to as IL2Ry) subunits of the IL-2 receptor (IL2R).

Certain methods for phenotypic and functional characterization of Treg cells including cells in which FoxP3 overexpression has been induced are known in the art (e.g., WO/2018/080541, WO/2019/210078, McMurchy et al., 2013 Meths. Mol. Biol. 946: 115-132; Thornton et al., 2019 Eur. J. Immunol. 49:398-412; Aarts-Riemens et al., 2008 Eur. J. Immunol. 38: 1381-1390; McGovern et al., 2017 Front. Immunol. 8: Art. 1517; which are each expressly incorporated by reference in its entirety) and are described herein. These and related methodologies are applicable to characterization of the present airT cells as described herein.

Unlike natural Treg cells, in the presently disclosed airT cells the intronic Treg-specific demethylated region (TSDR) in the FoxP3 gene locus comprises cytosine-guanine (CG) dinucleotides having cytosine (C) nucleotides at certain positions that are predominantly methylated. For example, in the present airT cells at least 80%, 85%, 90%, 95%, 96%, 97%, 98%, or 99% of the TSDR C nucleotides at nucleotide positions that comprise a demethylated C nucleotide in a naturally occurring Treg cell are methylated. Methylation analysis of the FoxP3 TSDR is known to be routine in the art by any of several different methodologies (e.g., Salazar et al., 2017 Front. Immunol. 8:219; Ngalamika et al., 2014 Immunol. Invest. 44(2): 126-136 which is expressly incorporated by reference in its entirety).

Despite this difference in TSDR epigenetic modification between airT cells and natural Treg cells, the present airT cells are capable of mounting an immunosuppressive response to TCR stimulation by a specifically recognized antigen. The antigen-specific immunosuppressive properties of the presently disclosed airT cells were therefore unexpected in view of the report by Wright et al. (2009 Proc. Nat. Acad. Sci. USA 106: 19078) that co-transfection of CD4+ cells with FoxP3 and TCR constructs in viral vectors did not produce Treg-like cells that were functionally capable of exhibiting antigen-specific suppression. Without wishing to be bound by theory, it is believed that the presently disclosed airT cells thus provide unforeseen advantages that may derive at least in part from the manner in which they are prepared, including by artificial gene editing as described herein.

In certain embodiments the present airT cell may be gene edited so as to express a FoxP3 gene product that is encoded by a FoxP3-encoding nucleotide sequence that is operably linked to a constitutive promoter, wherein constitutive expression of the FoxP3 gene product refers to a FOXP3 expression level that is equal to or greater than the FOXP3 expression level of a naturally occurring regulatory T (Treg) cell. In some embodiments, only a regulatory element (e.g., a promoter) is inserted in the FOXP3 locus (e.g., a constitutive promoter downstream from one or more naturally-occurring regulator elements (e.g., CNS sequences) or the TSDR, without the insertion of a gene-encoding sequence/nucleic acid. In some embodiments, a promoter is inserted upstream from the first coding exon of FOXP3. In certain preferred embodiments the constitutive promoter is the MND promoter, and in certain preferred embodiments the MND promoter has been knocked-in to the native FOXP3 gene locus by HDR gene editing. In certain embodiments the constitutively active promoter is knocked-in downstream of (e.g., 10 to 10,000 bp downstream from) an intronic regulatory T cell (Treg)-specific demethylation region (TSDR) in the native FOXP3 gene locus. In certain embodiments, a nucleic acid molecule comprising an exogenous FOXP3-encoding nucleic acid operably linked to the constitutive promoter is knocked-in by HDR gene editing to the native FOXP3 gene locus. In certain embodiments, a nucleic acid molecule comprising an exogenous FOXP3-encoding nucleic acid operably linked to the constitutive promoter is knocked-in by HDR gene editing at a chromosomal site other than the native FOXP3 gene locus, such as a TRAC gene locus or an AAVS1 locus or another gene locus. Accordingly, in these and related embodiments, the present disclosure for the first time teaches certain unexpected advantages that are associated with artificial gene editing by which FOXP3 gene expression is regulated by the constitutively active promoter, and in particularly preferred embodiment by the constitutively active MND promoter, for the production of the presently described engineered artificial immunoregulatory T (airT) cells.

### Gene Editing Methods

Any one of a number of gene- or genome- editing methods can used to accomplish editing of a first or second locus (or additional loci), e.g., the TRAC locus and/or a Foxp3 locus. Non-limiting gene editing methods include RNA-guided nuclease (RGN)-mediated gene editing, zinc finger nuclease (ZFN)-mediated gene editing, transcription activator-like effector nuclease (TALEN)-mediated gene editing, transposon-mediated gene editing, serine integrase-mediated gene editing, lentivirus-mediated gene editing, CRISPR/Cas-mediated gene editing, and homologous recombination-mediated gene editing. As used herein, the term “chromosomal gene knockout” refers to a genetic alteration, inactivation, or introduced inhibitory agent in a host cell that prevents (e.g., reduces, delays, suppresses, or abrogates) production, by the host cell, of a functionally active endogenous polypeptide product. Alterations resulting in a chromosomal gene knockout or inactivation can include, for example, introduced nonsense mutations (including the formation of premature stop codons), missense mutations, gene deletion, or strand breaks, as well as the heterologous expression of inhibitory nucleic acid molecules that inhibit endogenous gene expression in the host cell.

In certain embodiments, a chromosomal gene knock-out or gene knock-in (e.g., insertion) is made by chromosomal editing of a host cell. Chromosomal editing can be performed using, for example, endonucleases. As used herein “endonuclease” refers to an enzyme capable of catalyzing cleavage of a phosphodiester bond within a polynucleotide chain. In certain embodiments, an endonuclease is capable of cleaving a targeted gene thereby inactivating or “knocking out” the targeted gene. An endonuclease may be a naturally occurring, recombinant, genetically modified, or fusion endonuclease. Examples of endonucleases for use in gene editing include zinc finger nucleases (ZFN), TALE-nucleases (TALEN), CRISPR-Cas nucleases, meganucleases, or megaTALs.

The nucleic acid strand breaks caused by the endonuclease are typically double-strand breaks (DSB) that may be commonly repaired through the distinct mechanisms of homology directed repair (HDR) by homologous recombination, or by non-homologous end joining (NHEJ). (NHEJ: Ghezraoui et al., 2014 Mol Cell 55(6):829-842; HDR: Jasin and Rothstein, 2013 Cold Spring Harb Perspect Biol 5(11):a012740, PMID 24097900) During HDR/homologous recombination, a donor nucleic acid molecule may be used for a donor gene “knock-in”, for target gene “knock-out”, and optionally to inactivate a target gene through a donor gene knock in or target gene knock out event. NHEJ is an error-prone repair process that often results in changes to the DNA sequence at the site of the cleavage, e.g., a substitution, deletion, or addition of at least one nucleotide. NHEJ may be used to “knock-out” a target gene. HDR is favored by the presence of a donor template at the time of DSB formation and is a preferred gene editing mechanism according to certain herein described embodiments.

As used herein, a “zinc finger nuclease” (ZFN) refers to a fusion protein comprising a zinc finger DNA-binding domain fused to a non-specific DNA cleavage domain, such as a Fok1 endonuclease. Each zinc finger motif of about 30 amino acids binds to about 3 base pairs of DNA, and amino acids at certain residues can be changed to alter triplet sequence specificity (see, e.g., Desjarlais et al., Proc. Natl. Acad. Sci. 90:2256-2260, 1993; Wolfe et al., J. Mol. Biol. 285:1917-1934, 1999). Multiple zinc finger motifs can be linked in tandem to create binding specificity to desired DNA sequences, such as regions having a length ranging from about 9 to about 18 base pairs. By way of background, ZFNs mediate genome editing by catalyzing the formation of a site-specific DNA double strand break (DSB) in the genome, and targeted integration of a transgene comprising flanking sequences homologous to the genome at the site of DSB is facilitated by homology directed repair (HDR). Alternatively, a DSB generated by a ZFN can result in knock out of target gene via repair by non-homologous end joining (NHEJ), which is an error-prone cellular repair pathway that results in the insertion or deletion of nucleotides at the cleavage site. In certain embodiments, a gene knockout or inactivation comprises an insertion, a deletion, a mutation or a combination thereof, made using a ZFN molecule.

As used herein, a “transcription activator-like effector nuclease” (TALEN) refers to a fusion protein comprising a TALE DNA-binding domain and a DNA cleavage domain, such as a FokI endonuclease. A “TALE DNA binding domain” or “TALE” is composed of one or more TALE repeat domains/units, each generally having a highly conserved 33-35 amino acid sequence with divergent 12th and 13th amino acids. The TALE repeat domains are involved in binding of the TALE to a target DNA sequence. The divergent amino acid residues, referred to as the Repeat Variable Diresidue (RVD), correlate with specific nucleotide recognition. The natural (canonical) code for DNA recognition of these TALEs has been determined such that an HD (histidine-aspartic acid) sequence at positions 12 and 13 of the TALE leads to the TALE binding to cytosine (C), NG (asparagine-glycine) binds to a T nucleotide, NI (asparagine-isoleucine) to A, NN (asparagine-asparagine) binds to a G or A nucleotide, and NG (asparagine-glycine) binds to a T nucleotide. Non-canonical (atypical) RVDs are also known (see, e.g., U.S. Patent Publication No. US 2011/0301073, which atypical RVDs are incorporated by reference herein in their entirety). TALENs can be used to direct site-specific double-strand breaks (DSB) in the genome of T cells. Non-homologous end joining (NHEJ) ligates DNA from both sides of a double-strand break in which there is little or no sequence overlap for annealing, thereby introducing errors that knock out gene expression. Alternatively, homology directed repair (HDR) can introduce a transgene at the site of DSB providing homologous flanking sequences are present in the donor template containing the transgene. In certain embodiments, a gene knockout comprises an insertion, a deletion, a mutation or a combination thereof, and made using a TALEN molecule.

As used herein, a “clustered regularly interspaced short palindromic repeats/Cas” (CRISPR/Cas) nuclease system refers to a system that employs a CRISPR RNA (crRNA)-guided Cas nuclease to recognize target sites within a genome (known as protospacers) via base-pairing complementarity and then to cleave the DNA if a short, conserved protospacer associated motif (PAM) immediately follows 3′ of the complementary target sequence. CRISPR/Cas systems are classified into types (e.g., type I, type II, type III, and type V) based on the sequence and structure of the Cas nucleases. The crRNA-guided surveillance complexes in types I and III need multiple Cas subunits. Type II system, the most studied, comprises at least three components: an RNA-guided Cas9 nuclease, a crRNA, and a trans-acting crRNA (tracrRNA). The tracrRNA comprises a duplex forming region. A crRNA and a tracrRNA form a duplex that is capable of interacting with a Cas9 nuclease and guiding the Cas9/crRNA:tracrRNA complex to a specific site on the target DNA via Watson-Crick base-pairing between the spacer on the crRNA and the protospacer on the target DNA upstream from a PAM. Cas9 nuclease cleaves a double-stranded break within a region defined by the crRNA spacer. Repair by NHEJ results in insertions and/or deletions which disrupt expression of the targeted locus. Alternatively, a donor template transgene with homologous flanking sequences can be introduced at the site of DSB via homology directed repair (HDR). The crRNA and tracrRNA can be engineered into a single guide RNA (sgRNA or gRNA) (see, e.g., Jinek et al., Science 337:816-21, 2012). Further, the region of the guide RNA complementary to the target site can be altered or programed to target a desired sequence (Xie et al., PLOS One 9:e100448, 2014; U.S. Pat. Appl. Pub. No. US 2014/0068797, U.S. Pat. Appl. Pub. No. US 2014/0186843; U.S. Pat. No. 8,697,359, and PCT Publication No. WO 2015/071474; each of which is incorporated by reference). Non-limiting examples of CRISPR/Cas nucleases include Cas9, SaCas9, CjCas9, xCas9, C2C1, Cas13a/C2c2, C2c3, Cas13b, Cpf1, and variants thereof.

In certain embodiments, a gene knockout or inactivation comprises an insertion, a deletion, a mutation or a combination thereof, and made using a CRISPR/Cas nuclease system. US/2016/033377 which is expressly incorporated by reference in its entirety, teaches methods for enhancing endonuclease-based gene editing, including AAV-expressed guide RNAs for use in CRISPR/Cas (e.g., Cas9) gene editing systems. Exemplary gRNA sequences and methods of using the same to knock out endogenous genes that encode immune cell proteins include those described in Ren et al., Clin. Cancer Res. 23(9):2255-2266 (2017), the gRNAs, CAS9 DNAs, vectors, and gene knockout techniques of which are hereby expressly incorporated by reference in their entirety.

As used herein, a “meganuclease,” also referred to as a “homing endonuclease,” refers to an endodeoxyribonuclease characterized by a large recognition site (double stranded DNA sequences of about 12 to about 40 base pairs). Meganucleases can be divided into five families based on sequence and structure motifs: LAGLIDADG, GIY-YIG, HNH, His-Cys box and PD-(D/E)XK. Exemplary meganucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII, whose recognition sequences are known (see, e.g., U.S. Pat. Nos. 5,420,032 and 6,833,252; Belfort et al., Nucleic Acids Res. 25:3379-3388, 1997; Dujon et al., Gene 82:115-118, 1989; Perler et al., Nucleic Acids Res. 22:1125-1127, 1994; Jasin, Trends Genet. 12:224-228, 1996; Gimble et al., J. Mol. Biol. 263:163-180, 1996; Argast et al., J. Mol. Biol. 280:345-353, 1998).

In certain embodiments, naturally occurring meganucleases may be used to promote site-specific genome modification of a target selected from PD-1, LAG3, TIM3, CTLA4, TIGIT, FasL, an HLA-encoding gene, or a TCR component-encoding gene. In other embodiments, an engineered meganuclease having a novel binding specificity for a target gene is used for site-specific genome modification (see, e.g., Porteus et al., Nat. Biotechnol. 23:967-73, 2005; Sussman et al., J. Mol. Biol. 342:31-41, 2004; Epinat et al., Nucleic Acids Res. 31:2952-62, 2003; Chevalier et al., Molec. Cell 10:895-905, 2002; Ashworth et al., Nature 441:656-659, 2006; Paques et al., Curr. Gene Ther. 7:49-66, 2007; U.S. Patent Publication Nos. US 2007/0117128; US 2006/0206949; US 2006/0153826; US 2006/0078552; and US 2004/0002092). In further embodiments, a chromosomal gene knockout is generated using a homing endonuclease that has been modified with modular DNA binding domains of TALENs to make a fusion protein known as a megaTAL. MegaTALs can be utilized to not only knock-out or inactivate one or more target genes, but to also introduce (knock in) heterologous or exogenous polynucleotides when used in combination with an exogenous donor template encoding a polypeptide of interest.

A chromosomal gene knockout can be confirmed directly by DNA sequencing of the host immune cell following use of the knockout procedure or agent. Chromosomal gene knockouts can also be inferred from the absence of gene expression (e.g., the absence of an mRNA or polypeptide product encoded by the gene) following the knockout.

In certain embodiments, a chromosomal gene knockout or inactivation comprises a knockout or inactivation of a TCR component gene selected from a TCR α variable region gene, a TCR β variable region gene, a TCR constant region gene, or a combination thereof.

### Cells in which the Genome is Edited

### T Cells and TCRs

Provided herein are airT cells comprising gene editing or modification in more than one locus of the genome, e.g., a first and second locus. The modification may be insertion of only a regulator element along with a CISC component, or insertion of one or more regulatory elements and one or more payloads, along with a CISC component. In some embodiments, a modification comprises insertion of more than one CISC component. In some embodiments, the present cell, such as a CD4+, CD4+CD25+, CD8+, CD8+CD25+, CD3+, or NK1.1+ airT cell comprises an artificial modification of a FOXP3 gene as described herein and further comprises at least one transduced polynucleotide/nucleic acid encoding an antigen-specific T cell receptor (TCR) polypeptide. Preferably and in certain embodiments, the native TCR gene has been knocked out, for example by a targeted gene editing knock out in the TCR alpha (TRAC) gene locus. As used herein “knocked out” can refer to the inactivation of a gene and/or the gene product, for example, such as by deletion of the gene or a portion of the gene, by insertion of nucleic acids into the gene to interrupt transcription and/or translation of the gene and/or its product. Also preferably and in certain embodiments, the transduced polynucleotide/nucleic acid encoding the TCR has been knocked in by gene editing to a specific gene locus, such as the TRAC gene locus or another targeted locus.

The present airT cells thus comprise artificial immunoregulatory T cells that in preferred embodiments are produced by selective editing of one or more specific gene loci in T lymphocytes as described herein. In some embodiments, an airT cell shows properties different from the cell that is engineered to produce it. For example, an airT cell may be produced from a stem cell. Preferred T cells are of mammalian origin, for example, T cells obtained from humans, non-human primates (e.g., chimpanzees, macaques, gorillas, etc.), rodents (e.g., mice, rats, etc.), lagomorphs (e.g., rabbits, hares, pikas, etc.), ungulates (e.g., cattle, horses, pigs, sheep, etc.), or other mammals. In certain preferred embodiments the T cells are human T cells. In some embodiments, a cell as provided herein is a human cell. In some embodiments, a cell is a lymphocyte (e.g., a NK1.1+, CD3+, CD4+, or CD8+ cell). In some embodiments, the cell is a T cell, a precursor T cell, or a hematopoietic stem cell. In some embodiments, the cell is a CD4+ T cell (e.g., a FOXP3−CD4+ T cell or a FOXP3+CD4+ T cell) or a CD8+ T cell (e.g., a FOXP3−CD8+ T cell or a FOXP3+ CD8+ T cell). In some embodiments, the cell is an NK-T cell (e.g., a FOXP3− NK-T cell or a FOXP3+ NK-T cell). In some embodiments, the cell is a regulatory B (Breg) cell (e.g., a FOXP3− B cell or a FOXP3+ B cell). In some embodiments, the cell is a CD25− T cell. In some embodiments, the cell is a regulatory T (Treg) cell. Non-limiting examples of Treg cells are Tr1, Th3, CD8+CD28−, and Qa-1 restricted T cells. In some embodiments, the Treg cell is a FOXP3+ Treg cell. In some embodiments, the Treg cell expresses CTLA-4, LAG-3, CD25, CD39, neuropilin-1, galectin-1, and/or IL-2Ra on its surface. In some embodiments, the cell is ex vivo. In some embodiments, a cell is in vivo. In some embodiments, a cell as provided herein is an engineered cell. In some embodiments, an engineered cell is a cell in which one or more genes/loci are manipulated or edited (e.g., to stabilize expression of one or more genes). In some embodiments, an engineered cell comprises editing of the Foxp3 gene/locus, e.g., by inserting a promoter (in some embodiments, downstream of (e.g., 10 to 10,000 bp downstream from) one or more regulatory elements like the TSDR, and/or upstream from the first coding exon). In some embodiments, the cell is a human cell. In some embodiments, a cell as described here in is isolated from a biological sample. A biological sample may be a sample from a subject (e.g., a human subject) or a composition produced in a lab (e.g., a culture of cells). A biological sample obtained from a subject make be a liquid sample (e.g., blood or a fraction thereof, a bronchial lavage, cerebrospinal fluid, or urine), or a solid sample (e.g., a piece of tissue) In some embodiments, the cell is obtained from peripheral blood. In some embodiments, the cell is obtained from umbilical cord blood.

A T cell or T lymphocyte is an immune system cell that matures in the thymus and produces a T cell receptor (TCR), e.g., an antigen-specific heterodimeric cell surface receptor typically comprised of an alpha-beta heterodimer or a gamma-delta heterodimer. T cells of a given clonality typically express only a single TCR clonotype that recognizes a specific antigenic epitope presented by a syngeneic antigen-presenting cell in the context of a major histocompatibility complex-encoded determinant. T cells can be naïve (“TN”; not exposed to antigen; increased expression of CD62L, CCR7, CD28, CD3, CD127, and CD45RA, and decreased or no expression of CD45RO as compared to TCM (described herein)), memory T cells (TM) (antigen experienced and long-lived), including stem cell memory T cells, and effector cells (antigen-experienced, cytotoxic). TM can be further divided into subsets of central memory T cells (TCM, expresses CD62L, CCR7, CD28, CD95, CD45RO, and CD127) and effector memory T cells (TEM, express CD45RO, decreased expression of CD62L, CCR7, CD28, and CD45RA). Effector T cells (TE) refers to antigen-experienced CD8+ cytotoxic T lymphocytes that express CD45RA, have decreased expression of CD62L, CCR7, and CD28 as compared to TCM, and are positive for granzyme and perforin. Helper T cells (TH) are CD4+ cells that influence the activity of other immune cells by releasing cytokines. CD4+ T cells can activate and suppress an adaptive immune response, and which of those two functions is induced will depend on the presence of other cells and signals. T cells can be collected using known techniques, and the various subpopulations or combinations thereof can be enriched or depleted by known techniques, for example, using antibodies that specifically recognize one or more T cell surface phenotypic markers, by affinity binding to antibodies, flow cytometry, fluorescence activated cell sorting (FACS), or immunomagnetic bead selection. Other exemplary T cells include regulatory T cells (Treg, also known as suppressor T cells), such as CD4+ CD25+(Foxp3+) regulatory T cells and Treg17 cells, as well as Tr1, Th3, CD8+CD28−, or Qa-1 restricted T cells.

### T Cell Receptors (TCRs) and Chimeric Antigen Receptors (CARs)

Some embodiments of the methods provided herein comprise inserting a nucleic acid molecule encoding an antigen-specific receptor into the genome of a cell. Expression of a heterologous antigen-specific receptor allows the genetically modified cell to be activated when the heterologous antigen-specific receptor binds to its cognate antigen, such as a peptide-MHC complex in the case of a heterologous T cell receptor (TCR), or non-MHC-associated antigen in the case of a chimeric antigen receptor (CAR). A transduced polynucleotide encoding an exogenously sourced TCR or CAR to be expressed in an airT cell may involve artificial modification of a native TCR gene locus (e.g., TRAC) and/or may also involve artificial modification of a chromosomal site other than a native TCR gene locus, for example, gene editing by knock-in (insertion) of a nucleic acid molecule comprising an exogenous TCR-encoding nucleic acid at a chromosomal site other than a native TCR gene locus, such as the FOXP3 gene locus, ROSA26 locus, or AAVS1 or another gene locus.

The two gene editing events that give rise in certain embodiments to expression in the present airT cells, of the TCR or CAR gene product concomitant with the first CISC component and of the FoxP3 gene product concomitant with the second CISC component concomitant, may be designed to take place in different alleles of the same gene locus (e.g., bi-allelic dual editing), or at two different gene loci (e.g., two-loci dual editing). Exemplary TCR amino acid and encoding nucleotide sequences are disclosed herein (e.g., FIG.s. 136-144) for TCR that specifically recognize antigens associated with the pathogenesis of autoimmune, allergic, and/or inflammatory conditions. In some embodiments, the TCR polypeptide binds to an antigen selected from the group consisting of vimentin, aggrecan, cartilage intermediate layer protein (CILP), preproinsulin, islet-specific glucose-6-phosphatase catalytic subunit-related protein (IGRP), and enolase. In some embodiments, the TCR polypeptide binds to an antigen present in or derived from a microorganism present in the gut. In some embodiments, the TCR polypeptide binds to an epitope of the bacterial protein OmpC. In some embodiments, the TCR polypeptide binds to an epitope of GAD65, PPI, or ZNT8. In some embodiments, the TCR polypeptide binds to an epitope of the E2 component of pyruvate dehydrogenase complex (PDC-E2). In some embodiments, the TCR polypeptide binds to a nuclear antigen. In some embodiments, the TCR polypeptide binds to a mitochondrial antigen. In some embodiments, the TCR polypeptide binds to an antigen comprising an epitope having the amino acid sequence of any one of SEQ ID NOs 1363-1376 and 1408-1415.

In some embodiments, the TCR polypeptide comprises: a CD3 alpha polypeptide having the amino acid sequence of any one of SEQ ID NOs 1377-1390; and/or a CD3 beta polypeptide having the amino acid sequence of any one of SEQ ID NOs 1377-1390.

In some embodiments, the TCR specifically binds an islet-specific peptide, such as peptide of GAD65 or IGRP. In some embodiments, the TCR specifically binds a peptide of GAD65. In some embodiments, the TCR specifically binds a peptide of IGRP. In some embodiments, the TCR specifically binds a GAD65 epitope selected from the group consisting of GAD65113-132, GAD265-284, GAD273-292, and GAD305-324. In some embodiments, the TCR specifically binds an IGRP epitope selected from IGRP17-36, IGRP241-260, and IGRP305-324. In some embodiments, the TCR specifically binds a peptide of PPI. In some embodiments the TCR specifically binds the epitope PPI76-90. In some embodiments, the TCR specifically binds a peptide of ZNT8. In some embodiments, the TCR specifically binds the epitope ZNT8266-285.

In some embodiments, an inserted polynucleotide encodes a CAR comprising an antigen-binding domain that specifically binds an antigen associated with an autoimmune, allergic, and/or inflammatory condition. A “chimeric antigen receptor” (CAR) refers to a fusion protein that is engineered to contain two or more naturally occurring amino acid sequences, domains, or motifs, linked together in a way that does not occur naturally or does not occur naturally in a host cell, which fusion protein can function as a receptor when present on a surface of a cell such as a T cell. CARs can include an extracellular portion comprising an antigen-binding domain (e.g., obtained or derived from an immunoglobulin or immunoglobulin-like molecule, such as a TCR antigen binding domain derived or obtained from a TCR specific for an autoantigen, an allergen, or an inflammatory disease-associated antigen, a scFv derived or obtained from an antibody, or an antigen-binding domain derived or obtained from a killer immunoreceptor from an NK cell) linked to a transmembrane domain and one or more intracellular signaling domains (optionally containing co-stimulatory domain(s)) (see, e.g., Sadelain et al., Cancer Discov., 3(4):388 (2013); see also Harris and Kranz, Trends Pharmacol. Sci., 37(3):220 (2016), Stone et al., Cancer Immunol. Immunother., 63(11):1163 (2014), and Walseng et al., Scientific Reports 7:10713 (2017), which CAR constructs and methods of making the same are incorporated by reference herein). Methods and compostions disclosed in the following publication can be useful with embodiments provided herein: U.S. patent Ser. No. 10/865,242 which is incorporated by reference in its entirety.

In some embodiments, the CAR binds to an antigen selected from the group consisting of vimentin, aggrecan, cartilage intermediate layer protein (CILP), preproinsulin, islet-specific glucose-6-phosphatase catalytic subunit-related protein (IGRP), and enolase. In some embodiments, the CAR binds to an antigen comprising an epitope having the amino acid sequence of any one of SEQ ID NOs 1363-1376 and 1408-1415. In some embodiments, the CAR specifically binds an islet-specific peptide, such as GAD65 or IGRP. In some embodiments, the CAR specifically binds GAD65. In some embodiments, the CAR specifically binds IGRP. In some embodiments, the CAR specifically binds a GAD65 epitope selected from the group consisting of GAD65113-132, GAD265-284, GAD273-292, and GAD305-324. In some embodiments, the CAR specifically binds an IGRP epitope selected from IGRP17-36, IGRP241-260, and IGRP305-324. In some embodiments, the CAR specifically binds PPI. In some embodiments the CAR specifically binds the epitope PPI76-90. In some embodiments, the CAR specifically binds ZNT8. In some embodiments, the CAR specifically binds the epitope ZNT8266-285.

### Target Specificity, TCRs, and CARs

As used herein, “T cell receptor” (TCR) refers to an immunoglobulin superfamily member having a variable binding domain, a constant domain, a transmembrane region, and a short cytoplasmic tail; see, e. g., Janeway et al., Immunobiology: The Immune System in Health and Disease, 3rd Ed., Current Biology Publications, p. 433, 1997. The TCR is capable of specifically binding to an antigen peptide bound to a major histocompatibility complex encoded (MHC) receptor. A TCR can be found on the surface of a T cell or may be released into the extracellular milieu in soluble form, and generally is comprised of a heterodimer having α and β chains (also known as TCR α and TCRβ, respectively), or 7 and 6 chains (also known as TCRγ and TCRδ, respectively), each having chain-characteristic constant (C) regions and highly polymorphic variable (V) regions in which reside complementarity determining regions (CDR) that are largely responsible for specific antigen recognition and binding by the TCR. In certain embodiments, a nucleic acid encoding a TCR can be codon optimized to enhance expression in a particular host cell, such as, for example, a cell of the immune system, a hematopoietic stem cell, a T cell, a primary T cell, a T cell line, a NK cell, or a natural killer T cell (Scholten et al., Clin. Immunol. 119:135, 2006).

Exemplary T cells that can express TCRs encoded by heterologous genetic material introduced in the cells according to certain embodiments of this disclosure include CD4+ T cells, CD8+ T cells, and related subpopulations thereof (e.g., naïve, central memory, stem cell memory, effector memory). In preferred embodiments the exemplary T cells are CD4+ T cells, the TCR-encoding genetic material is introduced by gene editing (e.g., homology directed repair following a specifically targeted double-strand break in genomic DNA), and the TCR specifically recognizes an antigen associated with pathogenesis of an autoimmune condition, an allergic condition, or an inflammatory condition, such as the specific TCRs that are structurally defined herein or TCRs that specifically recognize the particular autoantigen, allergen, or inflammatory disease antigen T cell epitopes that are disclosed herein.

Like other antigen-binding members of the immunoglobulin superfamily (e.g., the immunoglobulins, also referred to as antibodies), the extracellular portion of TCR chains (e.g., α-chain, β-chain) contain two immunoglobulin domains, a variable domain (e.g., α-chain variable domain or Vα, β-chain variable domain or Vβ; typically amino acids 1 to 116 based on Kabat numbering (Kabat et al., “Sequences of Proteins of Immunological Interest, US Dept. Health and Human Services, Public Health Service National Institutes of Health, 1991, 5th ed.)) at the N-terminus, and one constant domain (e.g., α-chain constant domain or Cα, typically 5 amino acids 117 to 259 based on Kabat, β-chain constant domain or Cβ, typically amino acids 117 to 295 based on Kabat) adjacent the cell membrane. Also, like immunoglobulins, the variable domains contain complementary determining regions (CDRs) separated by framework regions (FRs) (see, e.g., Jores et al., Proc. Nat'l Acad. Sci. USA 87:9138, 1990; Chothia et al., EMBO J. 7:3745, 1988; see also Lefranc et al., Dev. Comp. Immunol. 27:55, 2003). The source of a TCR as used in the present disclosure may be from various animal species, such as a human, non-human primate, mouse, rat, rabbit, or other mammal.

The term “variable region” or “variable domain” refers to the structural domain of an immunoglobulin superfamily binding protein (e.g., a TCR α-chain or β-chain (or γ chain and δ chain for γδ TCRs)) that is involved in specific binding of the immunoglobulin superfamily binding protein (e.g., TCR) to antigen. The variable domains of the a chain and β chain (Vα and Vβ, respectively) of a native TCR generally have similar structures, with each domain comprising four generally conserved framework regions (FRs) and three CDRs. The Vα domain is encoded by two separate DNA segments, the variable gene segment and the joining gene segment (V-J); the Vβ domain is encoded by three separate DNA segments, the variable gene segment, the diversity gene segment, and the joining gene segment (V-D-J). A single Vα or Vβ domain may be sufficient to confer antigen-binding specificity. Furthermore, TCRs that bind a particular antigen may be isolated using a Vα or Vβ domain from a TCR that binds the antigen to screen a library of complementary Vα or Vβ domains, respectively.

The terms “complementarity determining region,” and “CDR,” are synonymous with “hypervariable region” or “HVR,” and are known in the art to refer to sequences of amino acids within immunoglobulin (e.g., TCR) variable regions, which confer antigen specificity and/or binding affinity and are separated from one another in primary amino acid sequence by framework regions. In general, there are three CDRs in each TCR α-chain variable region (αCDR1, αCDR2, αCDR3) and three CDRs in each TCR β-chain variable region (βCDR1, βCDR2, βCDR3). In TCRs, CDR3 is thought to be the main CDR responsible for recognizing processed antigen. In general, CDR1 and CDR2 interact mainly or exclusively with the MHC.

CDR1 and CDR2 are encoded within the variable gene segment of a TCR variable region-coding sequence, whereas CDR3 is encoded by the region spanning the variable and joining segments for Vα, or the region spanning variable, diversity, and joining segments for Vβ. Thus, if the identity of the variable gene segment of a Vα or Vβ is known, the sequences of their corresponding CDR1 and CDR2 can be deduced; e.g., according to a numbering scheme as described herein. Compared with CDR1 and CDR2, CDR3 is typically significantly more diverse due to the addition and loss of nucleotides during the recombination process.

TCR variable domain sequences can be aligned to a numbering scheme (e.g., Kabat, Chothia, EU, IMGT, Enhanced Chothia, and Aho), allowing equivalent residue positions to be annotated and for different molecules to be compared using, for example, ANARCI software tool (2016, Bioinformatics 15:298-300). A numbering scheme provides a standardized delineation of framework regions and CDRs in the TCR variable domains. In certain embodiments, a CDR of the present disclosure is identified according to the IMGT numbering scheme (Lefranc et al., Dev. Comp. Immunol. 27:55, 2003; imgt.org/IMGTindex/V-QUEST.php).

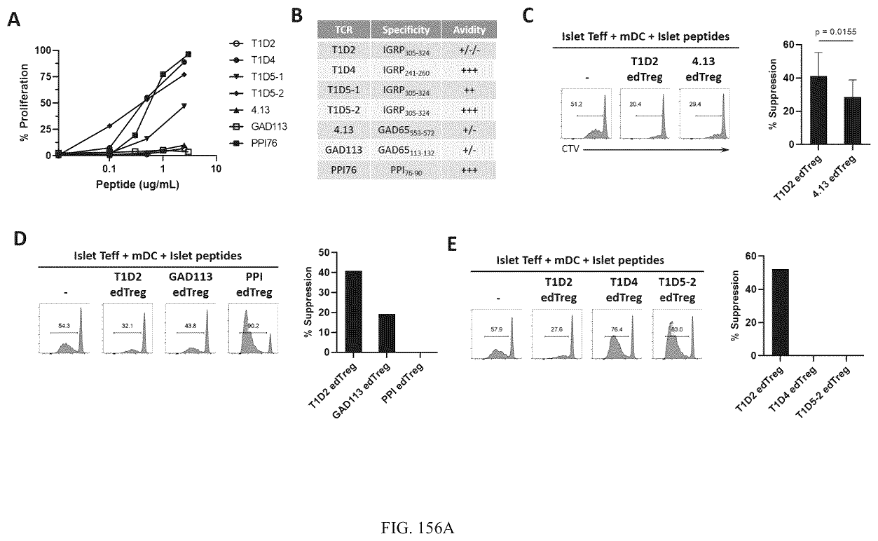

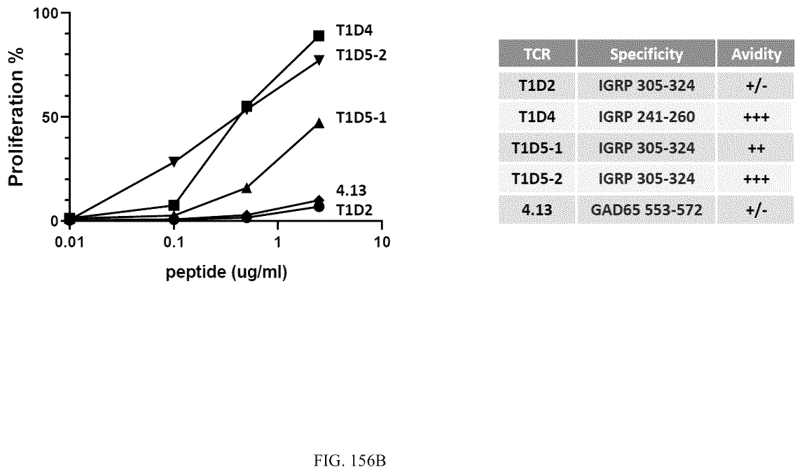

The ability of TCRs to specifically bind a cognate epitope or antigen may be described in terms of affinity, as described above, or avidity. As used herein, “avidity” can refer to a functional activity induced by an interaction between a TCR expressed on a T cell with an antigen, such as a MHC Class II-peptide complex comprising an antigen on an antigen presenting cell. Relative avidity of a TCR to an antigen can include the level of proliferation induced in a cell expressing the TCR when the TCR binds to the antigen. In one example, a dose-response curve and the measurement of proliferation as shown in FIG. 156A, panel A can be utilized to determine avidity of a TCR for an epitope or antigen. There are multiple ways to assess TCR avidity for a MHC-peptide complex. In one example, a functional definition of avidity can be based on the proliferative response of a T cell expressing the TCR of interest when co-cultured with an antigen presenting cell. In another example, proliferation is measured with dye dilution at 4-6 days. In another example, proliferation is measured with H3-Thymidine incorporation. In another example, proliferation is measured with expression of Ki67. In another example, relative avidity is determined by comparison to an established TCR known to be of high avidity.

As used herein, “CD4” is an immunoglobulin co-receptor glycoprotein that assists the TCR in communicating with antigen-presenting cells (see, Campbell & Reece, Biology 909 (Benjamin Cummings, Sixth Ed., 2002)). CD4 is found on the surface of immune cells such as T helper cells, monocytes, macrophages, and dendritic cells, and includes four immunoglobulin domains (D1 to D4) that are expressed at the cell surface. During antigen presentation, CD4 is recruited, along with the TCR complex, to bind to different regions of the MHC class II molecule (CD4 binds MHCII β2, while the TCR complex binds MHCII α1/β1). Without wishing to be bound by theory, it is believed that close proximity to the TCR complex allows CD4-associated kinase molecules to phosphorylate the immunoreceptor tyrosine activation motifs (ITAMs) present on the cytoplasmic domains of CD3. This activity is thought to amplify the signal generated by the activated TCR in order to produce or recruit various types of immune system cells, including T helper cells, and to promote immune responses.

As used herein “CD8” can refer to a transmembrane glycoprotein that serves as a co-receptor for the TCR. Along with the TCR, the CD8 co-receptor plays a role in T cell signaling and aiding with cytotoxic T cell antigen interactions. Like the TCR, CD8 binds to a MHC molecule, but is specific for the MHC class I protein. To function, CD8 forms a dimer, including a pair of CD8 chains. The most common form of CD8 is composed of a CD8-α and CD8-β chain. The extracellular IgV-like domain of CD8-α interacts with the a3 portion of the Class I MHC molecule. This affinity keeps the T cell receptor of the cytotoxic T cell and the target cell bound closely together during antigen-specific activation.

In certain embodiments, a TCR is found on the surface of T cells (or T lymphocytes) and associates with a CD3 complex. “CD3” is a multi-protein complex of six chains (see, Abbas and Lichtman, 2003; Janeway et al., p. 172 and 178, 1999) that is associated with antigen signaling in T cells. In mammals, the complex comprises a CD3γ chain, a CD36 chain, two CD3ε chains, and a homodimer of CD3ζ chains. The CD3γ, CD3β, and CD3ε chains are related cell surface proteins of the immunoglobulin superfamily containing a single immunoglobulin domain. The transmembrane regions of the CD3γ, CD3β, and CD3ε chains are negatively charged, which is believed to allow these chains to associate with positively charged regions of T cell receptor chains. The intracellular tails of the CD3γ, CD3β, and CD3ε chains each contain a single conserved motif known as an immunoreceptor tyrosine-based activation motif or ITAM, whereas each CD3ζ (chain has three such motifs. Without wishing to be bound by theory, it is believed that the ITAMs are important for the signaling capacity of a TCR complex. CD3 as used in the present disclosure may be from various animal species, including human, non-human primate, mouse, rat, or other mammals.

As used herein, “TCR complex” refers to a complex formed by the association of CD3 with TCR. For example, a TCR complex can be composed of a CD3γ chain, a CD3β chain, two CD3ε chains, a homodimer of CD3ζ (chains, a TCRα chain, and a TCRβ chain. Alternatively, a TCR complex can be composed of a CD3γ chain, a CD3β chain, two CD3ε chains, a homodimer of CD3ζ (chains, a TCRγ chain, and a TCRβ chain.

A “component of a TCR complex”, as used herein, refers to a TCR chain (i.e., TCRα, TCRβ, TCRγ or TCRδ), a CD3 chain (i.e., CD3γ, CD3δ, CD3ε or CD3ζ), or a complex formed by two or more TCR chains or CD3 chains (e.g., a complex of TCRα and TCRβ, a complex of TCRγ and TCRδ, a complex of CD3ε and CD3δ, a complex of CD3γ and CD3ε, or a sub-TCR complex of TCRα, TCRβ, CD3γ, CD3δ, and two CD3ε chains).

As used herein, “chimeric antigen receptor” (CAR) refers to a fusion protein that is engineered to contain two or more naturally occurring amino acid sequences, domains, or motifs, linked together in a way that does not occur naturally or does not occur naturally in a host cell, which fusion protein can function as a receptor when present on a surface of a cell such as a T cell. CARs can include an extracellular portion comprising an antigen-binding domain (e.g., obtained or derived from an immunoglobulin or immunoglobulin-like molecule, such as a TCR antigen binding domain derived or obtained from a TCR specific for an autoantigen, an allergen, or an inflammatory disease-associated antigen, a scFv derived or obtained from an antibody, or an antigen-binding domain derived or obtained from a killer immunoreceptor from an NK cell) linked to a transmembrane domain and one or more intracellular signaling domains (optionally containing co-stimulatory domain(s)) (see, e.g., Sadelain et al., Cancer Discov., 3(4):388 (2013); see also Harris and Kranz, Trends Pharmacol. Sci., 37(3):220 (2016), Stone et al., Cancer Immunol. Immunother., 63(11):1163 (2014), and Walseng et al., Scientific Reports 7:10713 (2017), which CAR constructs and methods of making the same are incorporated by reference herein). Methods and compostions disclosed in the following publication can be useful with embodiments provided herein: U.S. patent Ser. No. 10/865,242 which is incorporated by reference in its entirety.

Many polypeptides may, as encoded by a polynucleotide sequence, comprise a “signal peptide” (also known as a leader sequence, leader peptide, or transit peptide). Signal peptides target newly synthesized polypeptides to their appropriate location inside or outside the cell. A signal peptide may be removed from the polypeptide during biosynthesis or after subcellular localization or extracellular secretion of the polypeptide is completed. Polypeptides that have a signal peptide are referred to herein as a “pre-protein” and polypeptides having their signal peptide removed are referred to herein as “mature” proteins or polypeptides.

A “linker” refers to an amino acid sequence that connects two proteins, polypeptides, peptides, domains, regions, or motifs and may provide a spacer function compatible with interaction of the two sub-binding domains so that the resulting polypeptide retains a specific binding affinity (e.g., scTCR) to a target molecule or retains signaling activity (e.g., TCR complex). In certain embodiments, a linker is comprised of about two to about 35 amino acids or 2-35 amino acids, for instance, about four to about 20 amino acids or 4-20 amino acids, about eight to about 15 amino acids or 8-15 amino acids, about 15 to about 25 amino acids or 15-25 amino acids. Exemplary linkers include glycine-serine linkers as are known in the art.

Exemplary TCR V region sequences and nucleic acid sequences coding therefor are disclosed herein, including in the Examples and Drawings, for TCR that specifically recognize antigens associated with autoimmune, allergic, and inflammatory conditions as provided herein. Also disclosed herein, including in the Examples and Drawings, are polypeptide sequences containing TCR-recognized antigenic epitopes of antigens associated with autoimmune, allergic, and inflammatory conditions as provided herein.

An “antigen” typically refers to an immunogenic molecule that provokes an immune response. This immune response may involve production of antibodies that specifically bind to the antigen, activation of specific immunologically competent cells (e.g., T cells such as T-helper, T-effector, Treg, etc.), or both. Although an antigen may frequently be thought of as a “non-self” structure to which a host immune system responds by recognizing the antigen as foreign, in the present disclosure “antigen” is not intended to be so limited and may in certain embodiments also include any autoantigen, which refers to a “self” molecular, cellular, organ, or tissue structure to which a host immune system may react inappropriately in the context of autoimmune disease. An antigen (immunogenic molecule) may be, for example, a peptide, glycopeptide, polypeptide, glycopolypeptide, polynucleotide, polysaccharide, lipid or the like. It is readily apparent that an antigen can be synthesized artificially, produced recombinantly, or derived from a biological sample. Exemplary biological samples that can contain one or more antigens include tissue samples, cells, biological fluids, biopsies, primary cultures, or combinations thereof. Antigens can be produced by cells that have been modified or genetically engineered to express an antigen, or that endogenously (e.g., without modification or genetic engineering by human intervention) express a mutation or polymorphism that is immunogenic.

In certain embodiments a T cell as provided herein may be used as a host cell that may be modified to include one or more heterologous polynucleotides comprising regulatory sequences (e.g., promoters, enhancers, etc.) and/or nucleic acid sequences encoding a desired TCR and/or nucleic acid sequences encoding all or a portion of a FoxP3 transcription factor as described herein. Methods for transfecting/transducing T cells with desired nucleic acids have been described (e.g., U.S. Patent Application Pub. No. US 2004/0087025) as have adoptive transfer procedures using T cells of desired target-specificity (e.g., Schmitt et al., Hum. Gen. 20:1240, 2009; Dossett et al., Mol. Ther. 17:742, 2009; Till et al., Blood 112:2261, 2008; Wang et al., Hum. Gene Ther. 18:712, 2007; Kuball et al., Blood 109:2331, 2007; US 2011/0243972; US 2011/0189141; Leen et al., Ann. Rev. Immunol. 25:243, 2007), such that adaptation of these methodologies to the presently disclosed embodiments is contemplated, based on the teachings herein. Particularly preferred embodiments relate to artificial modification of a T cell genome by targeted gene editing as described herein.

Any appropriate method can be used to transfect or transduce the cells, for example, the T cells, or to administer the polynucleotides or compositions of the present methods. Known methods for delivering polynucleotides to host cells include, for example, use of cationic polymers, lipid-like molecules, and certain commercial products such as, for example, IN-VIVO-JET PEI. Other methods include ex vivo transduction, injection, electroporation, DEAE-dextran, sonication loading, liposome-mediated transfection, receptor-mediated transduction, microprojectile bombardment, transposon-mediated transfer, and the like. Transposon-based systems are described in Woodard L. E. et al. (2012) PLoS ONE 7(11): e42666; and Amberger M. et al., (2020) BioEssays 42: 20000136, which are each incorporated by reference in its entirety. Still further methods of transfecting or transducing host cells employ vectors, as also described herein and known to the art.

A nucleic acid may comprise DNA or RNA and may be wholly or partially synthetic. Reference to a nucleotide sequence as set out herein encompasses a DNA molecule with the specified sequence and encompasses an RNA molecule with the specified sequence in which U is substituted for T, unless context requires otherwise. The term “isolated polynucleotide” as used herein shall mean a polynucleotide of genomic, cDNA, or synthetic origin or some combination thereof, wherein by virtue of its origin the isolated polynucleotide (1) is not associated with all or a portion of a polynucleotide in which the isolated polynucleotide is found in nature, (2) is linked to a polynucleotide to which it is not linked in nature, or (3) does not occur in nature as part of a larger sequence. In some embodiments, a nucleic acid is comprised within a polynucleotide.

The term “operably linked” means that the components to which the term is applied are in a relationship that allows them to carry out their inherent functions under suitable conditions. For example, a transcription control sequence “operably linked” to a protein coding sequence is ligated thereto so that expression of the protein coding sequence is achieved under conditions compatible with the transcriptional activity of the control sequences.

The term “control sequence” as used herein refers to polynucleotide sequences that can affect expression, processing or intracellular localization of coding sequences to which they are ligated or operably linked. The nature of such control sequences may depend upon the host organism. In particular embodiments, transcription control sequences for prokaryotes may include a promoter, ribosomal binding site, and transcription termination sequence. In other particular embodiments, transcription control sequences for eukaryotes may include promoters comprising one or a plurality of recognition sites for transcription factors, transcription enhancer sequences, transcription termination sequences and polyadenylation sequences. In certain embodiments, “control sequences” can include leader sequences and/or fusion partner sequences.

The term “polynucleotide” as referred to herein means single-stranded or double-stranded nucleic acid polymers. In certain embodiments, the nucleotides comprising the polynucleotide can be ribonucleotides or deoxyribonucleotides or a modified form of either type of nucleotide. Such modifications may include base modifications such as bromouridine, ribose modifications such as arabinoside and 2′,3′-dideoxyribose and internucleotide linkage modifications such as phosphorothioate, phosphorodithioate, phosphoroselenoate, phosphorodiselenoate, phosphoroanilothioate, phoshoraniladate or phosphoroamidate. The term “polynucleotide” specifically includes single and double stranded forms of DNA. In some embodiments, a polynucleotide comprises one or more nucleic acids (e.g., a stretch of consecutives nucleotides or base pairs) that encode one or more polypeptides.

The term “naturally occurring nucleotides” includes deoxyribonucleotides and ribonucleotides. The term “modified nucleotides” includes nucleotides with modified or substituted sugar groups and the like. The term “oligonucleotide linkages” includes oligonucleotide linkages such as phosphorothioate, phosphorodithioate, phosphoroselenoate, phosphorodiselenoate, phosphoroanilothioate, phoshoraniladate, or phosphoroamidate, and the like. See, e.g., LaPlanche et al., 1986, Nucl. Acids Res., 14:9081; Stec et al., 1984, J. Am. Chem. Soc., 106:6077; Stein et al., 1988, Nucl. Acids Res., 16:3209; Zon et al., 1991, Anti-Cancer Drug Design, 6:539; Zon et al., 1991, OLIGONUCLEOTIDES AND ANALOGUES: A PRACTICAL APPROACH, pp. 87-108 (F. Eckstein, Ed.), Oxford University Press, Oxford England; Stec et al., U.S. Pat. No. 5,151,510; Uhlmann and Peyman, 1990, Chemical Reviews, 90:543, the disclosures of which are hereby expressly incorporated by reference in their entireties. An oligonucleotide can include a detectable label to enable detection of the oligonucleotide or hybridization thereof.

The term “vector” is used to refer to any molecule (e.g., nucleic acid, plasmid, or virus) used to transfer coding information to a host cell. The term “expression vector” refers to a vector that is suitable for transformation of a host cell and contains nucleic acid sequences that direct and/or control expression of inserted heterologous nucleic acid sequences. Expression includes, but is not limited to, processes such as transcription, translation, and RNA splicing, if introns are present. Non-limiting examples of vectors include artificial chromosomes, minigenes, cosmids, plasmids, phagemids, and viral vectors. Non-limiting examples of viral vectors include lentiviral vectors, retroviral vectors, herpesvirus vectors, adenovirus vectors, and adeno-associated viral vectors. In some embodiments, one or more vectors comprising nucleic acids for use in the methods provided herein are lentiviral vectors. In some embodiments, one or more vectors are adenoviral vectors. In some embodiments, one or more vectors are adeno-associated viral (AAV) vectors. In some embodiments, one or more AAV vectors are AAV5 vectors. In some embodiments, one or more AAV vectors are AAV6 vectors.

As will be understood by those skilled in the art, nucleic acid may include genomic sequences, extra-genomic and plasmid-encoded sequences and smaller engineered gene segments that express, or may be adapted to express, proteins, polypeptides, peptides and the like. Such segments may be naturally isolated or modified synthetically by the skilled person.

As will be also recognized by the skilled artisan, polynucleotides may be single-stranded (coding or antisense) or double-stranded, and may be DNA (genomic, cDNA or synthetic) or RNA molecules. RNA molecules may include HnRNA molecules, which contain introns and correspond to a DNA molecule in a one-to-one manner, and mRNA molecules, which do not contain introns. Additional coding or non-coding sequences may, but need not, be present within a polynucleotide according to the present disclosure, and a polynucleotide may, but need not, be linked to other molecules and/or support materials. Polynucleotides may comprise a native sequence or may comprise a sequence encoding a variant or derivative of such a sequence.

In other related embodiments, polynucleotide variants may have substantial identity to a polynucleotide sequence encoding an immunomodulatory polypeptide described herein. For example, a polynucleotide may be a polynucleotide comprising at least 70% sequence identity, preferably at least 75%, 80%, 85%, 90%, 95%, 96%, 97%, 98%, or 99% or higher, sequence identity or a sequence identity that is within a range defined by any two of the aforementioned percentages as compared to a reference polynucleotide sequence such as a sequence encoding an antibody described herein, using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

Typically, polynucleotide variants will contain one or more substitutions, additions, deletions and/or insertions, preferably such that the binding affinity of a polypeptide variant of a given polypeptide which is capable of a specific binding interaction with another molecule and is encoded by the variant polynucleotide is not substantially diminished relative to a polypeptide encoded by a polynucleotide sequence specifically set forth herein.

In certain other related embodiments, polynucleotide fragments may comprise or consist essentially of various lengths of contiguous stretches of sequence identical to or complementary to a sequence encoding a polypeptide as described herein. For example, polynucleotides are provided that comprise or consist essentially of at least or at least about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, 110, 120, 130, 140, 150, 200, 300, 400, 500 or 1000 or more contiguous nucleotides of a sequences the encodes a polypeptide, or variant thereof, disclosed herein, as well as, all intermediate lengths there between. It will be readily understood that “intermediate lengths”, in this context, means any length between the quoted values, such as 50, 51, 52, 53, etc.; 100, 101, 102, 103, etc.; 150, 151, 152, 153, etc.; including all integers through 200-500; 500-1,000, and the like. A polynucleotide sequence as described here may be extended at one or both ends by additional nucleotides not found in the native sequence. This additional sequence may consist of 1, 2, 3, 4, 5, 6, 7, 8, 9, 10, 11, 12, 13, 14, 15, 16, 17, 18, 19, or 20 nucleotides at either end of a polynucleotide encoding a polypeptide described herein or at both ends of a polynucleotide encoding a polypeptide described herein.

In another embodiment, polynucleotides are provided that are capable of hybridizing under moderate to high stringency conditions to a polynucleotide sequence encoding a polypeptide, or variant thereof, provided herein, or a fragment thereof, or a complementary sequence thereof. Hybridization techniques are well known in the art of molecular biology. For purposes of illustration, suitable moderately stringent conditions for testing the hybridization of a polynucleotide as provided herein with other polynucleotides include prewashing in a solution of 5× SSC, 0.5% SDS, 1.0 mM EDTA (pH 8.0); hybridizing at 50° C.-60° C., 5× SSC, overnight; followed by washing twice at 65° C. for 20 minutes with each of 2×, 0.5× and 0.2× SSC containing 0.1% SDS. One skilled in the art will understand that the stringency of hybridization can be readily manipulated, such as by altering the salt content of the hybridization solution and/or the temperature at which the hybridization is performed. For example, in another embodiment, suitable highly stringent hybridization conditions include those described above, with the exception that the temperature of hybridization is increased, e.g., to 60° C.-65° C. or 65° C. 70° C.

The polynucleotides described herein, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of or about of 10,000, 5000, 3000, 2,000, 1,000, 500, 200, 100, or 50 base pairs in length, and the like, (including all intermediate lengths) are contemplated to be useful.

When comparing polynucleotide or nucleic acid sequences, two sequences are said to be “identical” if the sequence of nucleotides in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least or at least about 20 contiguous positions, usually 30 to 75, or 40 to 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, WI), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J., Unified Approach to Alignment and Phylogenes, pp. 626-645 (1990); Methods in Enzymology vol. 183, Academic Press, Inc., San Diego, CA; Higgins, D. G. and Sharp, P. M., CABIOS 5:151-153 (1989); Myers, E. W. and Muller W., CABIOS 4:11-17 (1988); Robinson, E. D., Comb. Theor 11:105 (1971); Santou, N. Nes, M., Mol. Biol. Evol. 4:406-425 (1987); Sneath, P. H. A. and Sokal, R. R., Numerical Taxonomy—the Principles and Practice of Numerical Taxonomy, Freeman Press, San Francisco, CA (1973); Wilbur, W. J. and Lipman, D. J., Proc. Natl. Acad., Sci. USA 80:726-730 (1983).

Alternatively, optimal alignment of sequences for comparison may be conducted by the local identity algorithm of Smith and Waterman, Add. APL. Math 2:482 (1981), by the identity alignment algorithm of Needleman and Wunsch, J. Mol. Biol. 48:443 (1970), by the search for similarity methods of Pearson and Lipman, Proc. Natl. Acad. Sci. USA 85: 2444 (1988), by computerized implementations of these algorithms (GAP, BESTFIT, BLAST, FASTA, and TFASTA in the Wisconsin Genetics Software Package, Genetics Computer Group (GCG), 575 Science Dr., Madison, WI), or by inspection.

One preferred example of algorithms that are suitable for determining percent sequence identity and sequence similarity are the BLAST and BLAST 2.0 algorithms, which are described in Altschul et al., Nucl. Acids Res. 25:3389-3402 (1977), and Altschul et al., J. Mol. Biol. 215:403-410 (1990), respectively. BLAST and BLAST 2.0 can be used, for example with the parameters described herein, to determine percent sequence identity among two or more the polynucleotides. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff, Proc. Natl. Acad. Sci. USA 89:10915 (1989)) alignments, (B) of 50, expectation (E) of 10, M=5, N=−4 and a comparison of both strands.

In certain embodiments, the “percentage of sequence identity” is determined by comparing two optimally aligned sequences over a window of comparison of at least 20 positions, wherein the portion of the polynucleotide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases occurs in both sequences to yield the number of matched positions, dividing the number of matched positions by the total number of positions in the reference sequence (i.e., the window size) and multiplying the results by 100 to yield the percentage of sequence identity.

It will be appreciated by those of ordinary skill in the art that, as a result of the degeneracy of the genetic code, there are many nucleotide sequences that encode a FoxP3, TCR, or antigenic peptide as described herein, or an antibody that specifically binds to such a peptide, as described herein. Some of these polynucleotides bear minimal sequence identity to the nucleotide sequence of the native or original polynucleotide sequence that encode FoxP3, TCR, or antigenic polypeptides described herein. Nonetheless, polynucleotides that vary due to differences in codon usage are expressly contemplated by the present disclosure. In certain embodiments, sequences that have been codon-optimized for mammalian expression are specifically contemplated.

Therefore, in another embodiment of the invention, a mutagenesis approach, such as site-specific mutagenesis, may be employed for the preparation of variants and/or derivatives of the FoxP3, TCR, or antigenic polypeptides described herein. By this approach, specific modifications in a polypeptide sequence can be made through mutagenesis of the underlying polynucleotides or nucleic acids that encode them. These techniques provide a straightforward approach to prepare and test sequence variants, for example, incorporating one or more of the foregoing considerations, by introducing one or more nucleotide sequence changes into the polynucleotide.

Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

In certain embodiments, the inventors contemplate the mutagenesis of the polynucleotide sequences or nucleic acids that encode a FoxP3, TCR, or antigenic polypeptide disclosed herein, or a variant thereof, to alter one or more properties of the encoded polypeptide, such as (e.g., for TCR or antigenic peptides) the binding affinity of the peptide or the variant thereof for a cognate ligand, or (e.g., for FoxP3) the immunosuppressive effects. The techniques of site-specific mutagenesis are well-known in the art and are widely used to create variants of both polypeptides and polynucleotides. For example, site-specific mutagenesis is often used to alter a specific portion of a DNA molecule. In such embodiments, a primer comprising typically 14 to 25 nucleotides or about 14 to about 25 nucleotides or so in length is employed, with about 5 to about 10 residues or 5 to 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phage are readily commercially-available and their use is generally well-known to those skilled in the art. Double-stranded plasmids are also routinely employed in site directed mutagenesis that eliminates the step of transferring the gene of interest from a plasmid to a phage.

In general, site-directed mutagenesis in accordance herewith is performed by first obtaining a single-stranded vector or melting apart of two strands of a double-stranded vector that includes within its sequence a DNA sequence encoding the desired peptide. An oligonucleotide primer bearing the desired mutated sequence is prepared, generally synthetically. This primer is then annealed with the single-stranded vector and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. For example, recombinant vectors encoding the desired peptide sequence may be treated or contacted with mutagenic agents, such as hydroxylamine, to obtain sequence variants. Specific details regarding these methods and protocols are found in the teachings of Maloy et al., 1994; Segal, 1976; Prokop and Bajpai, 1991; Kuby, 1994; and Maniatis et al., 1982, each incorporated herein by reference, for that purpose.

As used herein, the term “oligonucleotide directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” is intended to refer to a process that involves the template-dependent extension of a primer molecule. The term template dependent process refers to nucleic acid synthesis of an RNA or a DNA molecule wherein the sequence of the newly synthesized strand of nucleic acid is dictated by the well-known rules of complementary base pairing (see, for example, Watson, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, expressly incorporated herein by reference in its entirety.

In another approach for the production of polypeptide variants, recursive sequence recombination, as described in U.S. Pat. No. 5,837,458, which is expressly incorporated by reference in its entirety, may be employed. In this approach, iterative cycles of recombination and screening or selection are performed to “evolve” individual polynucleotide variants having, for example, increased binding affinity. Certain embodiments also provide constructs in the form of plasmids, vectors, transcription or expression cassettes which comprise at least one polynucleotide as described herein.

It will be appreciated that the practice of the several embodiments of the present invention will employ, unless indicated specifically to the contrary, conventional methods in virology, immunology, microbiology, molecular biology and recombinant DNA techniques that are within the skill of the art, and many of which are described below for the purpose of illustration. Such techniques are explained fully in the literature. See, e.g., Current Protocols in Molecular Biology or Current Protocols in Immunology, John Wiley & Sons, New York, N.Y. (2009); Ausubel et al., Short Protocols in Molecular Biology, 3rd ed., Wiley & Sons, 1995; Sambrook and Russell, Molecular Cloning: A Laboratory Manual (3rd Edition, 2001); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984) which are each incorporated by reference in its entirety.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references that are cited and discussed throughout the present specification. Unless specific definitions are provided, the nomenclature utilized in connection with, and the laboratory procedures and techniques of, molecular biology, analytical chemistry, synthetic organic chemistry, and medicinal and pharmaceutical chemistry described herein are those well-known and commonly used in the art. Standard techniques may be used for recombinant technology, molecular biological, microbiological, chemical syntheses, chemical analyses, pharmaceutical preparation, formulation, and delivery, and treatment of patients.

### Autoimmune, Allergic, and Inflammatory Conditions and Associated Antigens

As noted above, the present airT cells may find uses in the treatment, inhibition, and/or amelioration of certain autoimmune, allergic, and inflammatory conditions. Clinical signs and symptoms of, and diagnostic criteria for, such conditions are known in the art. Non-limiting examples of such conditions for which the present airT cells may be beneficially administered to a human patient or other mammalian host in need of antigen-specific immunosuppression, which may refer to an individual in whom there may be present a clinically inappropriate array of pro-inflammatory mediators (e.g., cytokines, lymphokines, hormones, and the like) and/or locally or systemically elevated levels of inflammatory cells, include: type 1 diabetes mellitus, multiple sclerosis, systemic lupus erythematosus, myasthenia gravis, rheumatoid arthritis, Crohn's disease, inflammatory bowel disease, bullous pemphigoid, pemphigus vulgaris, autoimmune hepatitis, asthma, allergy (e.g., specific hypersensitivity to food, plant, animal, environmental, drug, chemical, or other allergens), tolerance induction for transplantation (e.g., pancreatic islet cell transplantation), or graft-versus-host disease (GVHD) following stem cell (e.g., hematopoietic SC) transplantation, and the like. In some embodiments, airT cells are used for treating primary biliary cholangitis. In some embodiments, airT cells are used for treating primary sclerosing cholangitis. In some embodiments, airT cells are used for treating autoimmune hepatitis. In some embodiments, airT cells are used for treating type 1 diabetes. In some embodiments, airT cells are used for treating islet cell transplantation. In some embodiments, airT cells are used for treating transplant rejection. In some embodiments, airT cells are used for treating multiple sclerosis. In some embodiments, airT cells are used for treating inflammatory bowel disease. In some embodiments, airT cells are used for treating acute respiratory distress syndrome. In some embodiments, airT cells are used for treating stroke. In some embodiments, airT cells are used for treating graft-versus-host disease.

Antigens associated with these conditions, and in particular, portions of such antigens in which epitopes recognized by TCR reside, are known and are set forth in the Drawings. Also set forth in the Drawings are TCR V-region sequences of TCR that have been described on the basis of their ability to recognize the herein disclosed antigens associated with autoimmune, allergic, and inflammatory conditions.

Methods for the identification and characterization of antigens associated with pathogenesis of an autoimmune condition, an allergic condition, or an inflammatory condition, including determination of autoreactive T cell epitopes, are known in the art. For instance, exemplary methods for identifying pancreatic islet autoantigenic polypeptides, including peptide fragments thereof that are recognized by T cells from type 1 diabetes (T1D) subjects, are described in Cerosaletti et al. (2017 J. Immunol. 199:323, which is expressly incorporated by reference in its entirety). Other polypeptide antigens associated with pathogenesis of an autoimmune condition, an allergic condition, or an inflammatory condition, including determination of autoreactive T cell epitopes, are disclosed herein including in the Drawings.

Cerosaletti et al. (2017) also describe exemplary and non-limiting methodologies for determining the structures of T cell receptors (TCR) that recognize antigens associated with pathogenesis of an autoimmune condition, an allergic condition, or an inflammatory condition. TCR structural features, including partial or complete TCR alpha chain variable (V-alpha) and/or beta chain variable (V-beta) region amino acid sequences and encoding polynucleotide sequences therefor are disclosed herein including in the Drawings for a variety of TCRs specific for different antigens associated with pathogenesis of an autoimmune condition, an allergic condition, or an inflammatory condition.

### Compositions and Methods of Use

Accordingly, certain presently disclosed embodiments contemplate administration of the herein described airT cells as adoptively transferred immunotherapeutic cells to provide antigen-specific immunosuppression for such a condition in which excessive and/or clinically deleterious antigen-specific immune activity is present. For example, by way of illustration and not limitation, according to certain embodiments there are contemplated immunotherapeutic protocols involving the adoptive transfer to a subject (e.g., a patient having an autoimmune, allergic, or other inflammatory condition) of the presently disclosed airT cells. Adoptive transfer protocols using unselected or selected T cells are known in the art (e.g., Schmitt et al., 2009 Hum. Gen. 20:1240; Dossett et al., 2009 Mol. Ther. 17:742; Till et al., 2008 Blood 112:2261; Wang et al., 2007 Hum. Gene Ther. 18:712; Kuball et al., 2007 Blood 109:2331; US2011/0243972; US2011/0189141; Leen et al., 2007 Ann. Rev. Immunol. 25:243; US2011/0052530, US2010/0310534; Ho et al., 2006 J. Imm. Meth. 310:40; Ho et al., 2003 Canc. Cell 3:431) and may be modified according to the teachings herein for use with transfer cell populations containing desired airT cells generated as described herein.

Administration of the airT cells can be carried out via any of the accepted modes of administration of agents for serving similar utilities. The airT cells can be prepared in a pharmaceutical composition by combining with an appropriate physiologically acceptable carrier, diluent or excipient, such as an aqueous liquid optionally containing suitable salts, buffers and/or stabilizers. Administration of airT cells may be achieved by a variety of different routes such as intravenous, intrahepatic, intraperitoneal, intragastric, intraarticular, intrathecal, or other routes, and in preferred embodiments by intravenous infusion.

A dose of airT cells to be administered to a subject may contain any number of cells that are therapeutically effective. In some embodiments a composition administered to a subject comprises 10³-10¹⁵ airT cells. In some embodiments, a composition administered to a subject comprises at least 10³ cells. In some embodiments a composition administered to a subject comprises at least 10 ml of fluid, or 10-200 ml of fluid.

In some embodiments, any of the methods of treating a disease or condition as disclosed herein, comprises contacting of any one of an airT cell as disclosed herein with a ligand that binds to two CISC components as described herein (e.g., rapamycin or a rapalog). In some embodiments, the contacting of an airT cell with the ligand is performed ex vivo, e.g., to select cells. In some embodiments, contacting of a cell with rapamycin or a rapalog is performed in vivo, e.g., to activate or maintain activity of a cell via the CISC components in the airT cell, and/or to inhibit activity of cells that do not express both CISC components. In some embodiments, cells comprising CISC machinery as described herein and rapamycin or a rapalog are administered to a subject simultaneously. In some embodiments, cells comprising CISC machinery as described herein and rapamycin or a rapalog are administered to a subject sequentially. Thus, in some embodiments, airT cells (e.g., that are administered to a subject) have one or more of the following three mechanisms/activities: (i) expression of Foxp3 that is not diminished under inflammatory conditions (achieved either insertion of a promoter or a promoter and gene-encoding sequence), (ii) expression of a TCR (e.g., in the TRAC locus), and (iii) activity of IL-2 signaling that is driven by CISC components (e.g., a split CISC that is split into being present in the Foxp3 gene/locus and the TCR-encoding gene/locus).

### Methods of Treatment

The present antigen-specific immunoregulatory T (airT) cell compositions and methods will, in certain embodiments, find uses in the treatment and/or amelioration of certain autoimmune conditions, allergic conditions, and/or inflammatory conditions, including in adoptively transferable immunotherapy, where stable airT cell viability and maintenance of antigen-specific immunoregulatory function provide unprecedented benefits.

In certain embodiments the airT cell described herein is unexpectedly capable of inducing an antigen-specific immunosuppressive response when stimulated by an antigen associated with pathogenesis of an autoimmune condition, an allergic condition, or an inflammatory condition such as one of the antigens disclosed herein. Such antigen-specifically induced immunosuppression may comprise one or more of: (i) inhibition of either or both of activation and proliferation of effector T cells that recognize the antigen that is specifically recognized by the airT TCR comprising the TCR polypeptide that is encoded by the at least one transduced polynucleotide, (ii) inhibition of expression of inflammatory cytokines or inflammatory mediators by effector T cells that recognize the antigen that is specifically recognized by the airT TCR comprising the TCR polypeptide that is encoded by the at least one transduced polynucleotide (iii) elaboration of one or more immunosuppressive cytokines or anti-inflammatory products, for example, elaboration of one or more inhibitory mechanisms including release of immunosuppressive cytokines or perforin/granzyme, induction of indoleamine 2,3-dioxygenase (IDO), competition for IL2 or adenosine, catabolism of tryptophan, and expression of inhibitory receptors by the airT cell, by the airT cell, and (iv) inhibition of either of or both of activation and proliferation of effector T cells that do not recognize the antigen that is specifically recognized by the airT TCR comprising the TCR polypeptide that is encoded by the at least one transduced polynucleotide, and (v) inhibition of expression of inflammatory cytokines or inflammatory mediators by effector T cells that do not recognize the antigen that is specifically recognized by the airT TCR comprising the TCR polypeptide that is encoded by the at least one transduced polynucleotide, or (vi). In some embodiments such antigenic stimulation of the airT cell is HLA-restricted.

In some embodiments, the generation of the present airT cells, which stably express FoxP3 as described herein, overcomes certain disadvantages associated with prior methodologies in which FOXP3 transgene expression was achieved by retroviral or lentiviral gene transfer. The resulting virally FoxP3-transduced cell populations were genetically heterogeneous by virtue of having randomly integrated FOXP3 transgenes of varying stability and varying expression levels at various genomic sites. Despite at least transiently exhibiting Treg characteristics such as phenotypic markers and cytokine expression profile, such transduced populations were also potentially compromised by carrying a concomitant risk of genotoxicity, as well as vulnerability to silencing by local regulatory elements at sites of viral integration.

To avoid these risks, some embodiments provided herein include the use of specifically targeted gene editing for artificial modification of the FOXP3 gene instead of relying on viral FOXP3 gene transfer and, optionally specifically targeted TCR gene editing. Certain embodiments described herein utilize lentiviral gene delivery to introduce candidate autoimmune-related TCRs (or CARs) into T cells, such as CD4 T cells or CD8 T cells, followed by FOXP3 gene editing of the cells to force stable FoxP3 expression. In some related embodiments, this approach is combined with any one of the TRAC locus editing methods disclosed herein, e.g., gene editing methods to simultaneously delete the endogenous TCR gene (e.g., via inactivation, also referred to herein as “knockout”).

Provided herein are methods comprising administering to a subject a composition of any one the airT cells as described herein. In some embodiments, a method comprises administering to a subject any one of the airT cells as described herein that are made using cells that are isolated from that subject (i.e., autologous cells). In some embodiments, a method comprises administering to a subject any one of the airT cells as described herein that are made using cells that are isolated from a subject that is different from the subject into which the cells are adminstered (e.g, allogeneic cells). In some embodiments, a method comprises (a) isolation of cells from a first subject, (b) contacting the cells with a first polynucleotide and a second polynucleotide respectively comprising a first CISC component and a second CISC component to produce airT cells, and (c) administering to a second subject the airT cells. In some embodiments, the first subject is the same as the second subject. In some embodiments, the first subject is different from the second subject. In some embodiments, the method further comprises administering to the first subject (e.g, before, simultaneously, or subsequently) a ligand that binds to the first and second CISC components. In some embodiments, the ligand is rapamycin or a rapalog.

In some embodiments, a method comprises administering airT cells to the subject more than once. In some embodiments, airT cells are administered to the subject 2, 3, 4, 5, 6, 7, 8, 9, 10, 11, 12, 13, 14, 15, 16, 17, 18, 19, 20, or more times. In some embodiments, airT cells are administered to a subject that has been diagnosed with (i) an autoimmune condition selected from type 1 diabetes mellitus, multiple sclerosis, systemic lupus erythematosus, myasthenia gravis, rheumatoid arthritis, early onset rheumatoid arthritis, ankylosing spondylitis, immune-mediated pregnancy loss, immune-mediated recurrent pregnancy loss, dermatomyositis psoriatic arthritis, Crohn's disease, inflammatory bowel disease (IBD), ulcerative colitis, bullous pemphigoid, pemphigus vulgaris, autoimmune hepatitis, psoriasis, Sjogren's syndrome, and celiac disease; (ii) an allergic condition selected from allergic asthma, steroid-resistant asthma, atopic dermatitis, celiac disease, pollen allergy, food allergy, drug hypersensitivity, and contact dermatitis; and/or (iii) an inflammatory condition selected from pancreatic islet cell transplantation, asthma, hepatitis, traumatic brain injury, primary sclerosing cholangitis, primary biliary cholangitis, polymyositis, stroke, Still's disease, acute respiratory distress syndrome, uveitis inflammatory bowel disease (IBD), ulcerative colitis, graft-versus-host disease (GVHD), tolerance induction for transplantation, transplant rejection, and sepsis. In some embodiments, the inflammatory condition is primary biliary cholangitis. In some embodiments, the inflammatory condition is primary sclerosing cholangitis. In some embodiments, the inflammatory condition is autoimmune hepatitis. In some embodiments, the autoimmune condition is type 1 diabetes. In some embodiments, the inflammatory condition is islet cell transplantation. In some embodiments, the inflammatory condition is transplant rejection. In some embodiments, the autoimmune condition is multiple sclerosis. In some embodiments, the inflammatory condition is inflammatory bowel disease. In some embodiments, the inflammatory condition is acute respiratory distress syndrome. In some embodiments, the inflammatory condition is stroke. In some embodiments, the inflammatory condition is graft-versus-host disease.

Preferred modes of administration depend upon the nature of the condition to be treated or prevented, which in certain embodiments will refer to a deleterious or clinically undesirable condition the extent, severity, likelihood of occurrence and/or duration of which may be decreased (e.g., reduced in a statistically significant manner relative to an appropriate control situation such as an untreated control) according to certain methods provided herein. An amount that, following administration, detectably reduces, inhibits, or delays such a condition, for instance, the local or global level autoimmune, allergic, or other harmful inflammatory activity, is considered effective. Persons skilled in the relevant arts will be familiar with any number of diagnostic, surgical and other clinical criteria to which can be adapted to evaluation of the effects of administration by adoptive transfer of the immunoregulatory airT cell compositions described herein. See, e.g., Humar et al., Atlas of Organ Transplantation, 2006, Springer; Kuo et al., Comprehensive Atlas of Transplantation, 2004 Lippincott, Williams & Wilkins; Gruessner et al., Living Donor Organ Transplantation, 2007 McGraw-Hill Professional; Antin et al., Manual of Stem Cell and Bone Marrow Transplantation, 2009 Cambridge University Press; Wingard et al. (Ed.), Hematopoietic Stem Cell Transplantation: A Handbook for Clinicians, 2009 American Association of Blood Banks.

Accordingly, in some embodiments the airT cell may express an antigen-specific T cell receptor (TCR) that comprises the antigen-specific TCR polypeptide encoded by the at least one transduced polynucleotide that encodes said TCR polypeptide and is capable of antigen-specifically induced immunosuppression in response to HLA-restricted stimulation by an antigen that is specifically recognized by the TCR polypeptide. Determination of the presence of immunosuppression may be accomplished by any of a wide variety of criteria with which those skilled in the art will be familiar. See, e.g., Sakaguchi et al., 2020 Ann. Rev. Immunol. 38:541 which is expressly incorporated by reference in its entirety.

For example, by way of illustration and not limitation, multiple mechanisms contributing to suppressive phenotype of Treg cells have been described, such as CTLA-4 immune checkpoint, expression of immunosuppressive cytokines such as IL-10 and TGF-β, cytotoxicity of target cells through the perforin/granzyme pathway, induction of indoleamine 2,3-dioxygenase (IDO) and the catabolism of tryptophan in target cells, as well as consumption of adenosine by expression of CD73, and competition with effector T (Teff) cells for IL-2 since Treg cells constitutively express CD25 (the a subunit of the high affinity receptor for IL-2). See, e.g., Verbsky, J. W., and Chatila, T. A. (2014). Chapter 23—Immune Dysregulation Leading to Chronic Autoimmunity. In Stiehm's Immune Deficiencies, K. E. Sullivan, and E. R. Stiehm, eds., (Amsterdam: Academic Press), pp. 497-516; Campbell et al. 2020 Cell Metab. 31(1):18-25; Dominguez-Villar et al., 2018 Nat. Immunol. 19:665-673; Sakaguchi et al., 2008 Cell 133(5):775-787.

In some embodiments, antigen-specifically induced immunosuppression thus may comprise one or more of: (i) inhibition of either or both of activation and proliferation of effector T cells that recognize the antigen that is specifically recognized by the airT TCR comprising the TCR polypeptide that is encoded by the at least one transduced polynucleotide, (ii) inhibition of expression of inflammatory cytokines or inflammatory mediators by effector T cells that recognize the antigen that is specifically recognized by the airT TCR comprising the TCR polypeptide that is encoded by the at least one transduced polynucleotide (iii) elaboration of one or more immunosuppressive cytokines or anti-inflammatory products, for example, elaboration of one or more inhibitory mechanisms including release of immunosuppressive cytokines or perforin/granzyme, induction of indoleamine 2,3-dioxygenase (IDO), competition for IL2 or adenosine, catabolism of tryptophan, and/or expression of inhibitory receptors by the airT cell, by the airT cell, or (iv) inhibition of either or both of activation and proliferation of effector T cells that do not recognize the antigen that is specifically recognized by the airT TCR comprising the TCR polypeptide that is encoded by the at least one transduced polynucleotide.

In certain instances, adoptive transfer airT cell immunotherapy doses (and optionally, at least one other therapeutic agent dose) may be provided between 1 day and 14 days over a 30 day period. In certain instances, doses (and optionally, at least one other therapeutic agent dose) may be provided 1, 2, 3, 4, 5, 6, 7, 8, 9, 10, 11, 12, 13, or 14 days over a 60 day period. Alternate protocols may be appropriate for individual subjects. A suitable dose is an amount of a compound that, when administered as described above, is capable of detectably altering or ameliorating symptoms, or decreases at least one indicator of autoimmune, allergic or other inflammatory immune activity in a statistically significant manner by at least 10-50% relative to the basal (e.g., untreated) level, which can be monitored by measuring specific levels of blood components, for example, detectable levels of circulating immunocytes and/or other inflammatory cells and/or soluble inflammatory mediators including proinflammatory cytokines.

In some embodiments, rapamycin or a rapalog is administered to the subject before the administration of airT cells, in conjunction with airT cells, and/or following the administration of airT cells. Administration of rapamycin or a rapalog that is capable of inducing dimerization of the CISC components on the surface of an airT cell results in continued IL-2 signal transduction in vivo, promoting survival and proliferation of the CISC-expressing cell without the undesired effects that would be caused by IL-2 administration, such as activation of other T cells. In some embodiments, the rapamycin or rapalog that is administered is everolimus, CCI-779, C20-methallylrapamycin, C16-(S)-3-methylindolerapamycin, C16-iRap, C16-(S)-7-methylindolerapamycin, AP21967, C16-(S)Butylsulfonamidorapamycin, AP23050, sodium mycophenolic acid, benidipine hydrochloride, AP1903, and AP23573, or a metabolite or derivative thereof. In some embodiments, the rapamycin or rapalog is administered at a dose of 0.001 mg/kg to 10 mg/kg body mass of the subject, or a dose between 0.001 mg/kg and 10 mg/kg. In some embodiments, the rapamycin or rapalog is administered at a dose of 0.001 mg/kg to 0.01 mg/kg, 0.01 mg/kg to 0.1 mg/kg, 0.1 mg/kg to 1 mg/kg, or 1 mg/kg to 10 mg/kg. In some embodiments, the rapamycin or rapalog is administered in a separate composition from the airT cells. In some embodiments, the rapamycin or rapalog is administered in multiple doses. In some embodiments, the rapamycin or rapalog is administered for 1, 2, 3, 4, 5, 6, 7, 8, 9, 10, 11, 12, 13, or 14 or more days. In some embodiments, the rapamycin or rapalog is administered for 1, 2, 3, 4, 5, 6, 7, 8, 9, 10, 11, 12, 13, 14, 15, 16, 17, 18, 19, or 20 or more weeks. In some embodiments, the subject is a human. In some embodiments, the administration of the rapamycin or rapalog results in prolonged survival of the administered cells, relative to a subject that is not administered rapamycin or a rapalog. In some embodiments, the administration of the rapamycin or rapalog increases the frequency of airT cells circulating in the peripheral blood of a subject, relative to a subject that is not administered rapamycin or a rapalog.

In general, an appropriate dosage and treatment regimen provides the airT cells in an amount sufficient to provide therapeutic and/or prophylactic benefit. Such a response can be monitored by establishing an improved clinical outcome (e.g., more frequent remissions, complete or partial, or longer disease-free survival) in treated subjects as compared to non-treated subjects. Decreases (e.g., reductions having statistical significance when compared to a relevant control) in preexisting immune responses to an antigen associated with an autoimmune, allergic, or other inflammatory condition as provided herein generally correlate with an improved clinical outcome. Such immune responses may generally be evaluated using standard leukocyte and/or lymphocyte cell surface marker or cytokine expression, proliferation, cytotoxicity or released cytokine assays, which are routine in the art and may be performed using samples obtained from a subject before and after therapy.

For example, an amount of airT cells that is administered is sufficient to result in clinically relevant reduction (e.g., a decrease that is clinically remarkable, preferably as may be detectable in a statistically significant manner relative to an appropriate control condition) in symptoms of autoimmune diseases, including but not limited to type 1 diabetes, rheumatoid arthritis (RA), systemic lupus erythematosus (SLE), multiple sclerosis, inflammatory bowel disease (IBD), psoriatic arthritis, Crohn's disease, ulcerative colitis, seronegative spondyloarthropathies, Behcet's disease, vasculitis, or other autoimmune diseases.

Accordingly, in some embodiments a reduction in one or more relevant clinical criteria as known in the art for assessing type 1 diabetes (T1D) may be identified following adoptive transfer, to a T1D patient, of airT cells expressing a TCR that specifically recognizes an epitope of an antigen having relevance to a T1D-associated autoantigen. Exemplary T1D-associated antigens and TCR structures that specifically recognize such antigens, which are typically autoantigens, are described herein.

Common defining criteria for stage two T1D may include detection of two or more pancreatic islet-specific autoantibodies in the patient and evidence of dysglycemia during an oral glucose-tolerance test. Dysglycemia may in some embodiments be defined as a fasting glucose level of 110 to 125 mg per deciliter (6.1 to 6.9 mmol per liter), a two-hour postprandial plasma glucose level of at least 140 mg per deciliter (7.8 mmol per liter) and less than 200 mg per deciliter (11.1 mmol per liter), or an intervening postprandial glucose level at 30, 60, or 90 minutes of greater than 200 mg per deciliter. In some embodiments clinical T1D may be defined as the presence of symptoms of diabetes (e.g., increased thirst, increased urination, and/or unexplained weight loss, compared to normal subjects known to be free of any risk for having or presence of T1D) and a blood sugar level equal to or greater than 200 milligrams per deciliter (mg/dL), a fasting blood sugar level that is equal to or greater than 126 mg/dL, or a two-hour oral glucose tolerance test (OGTT) result that is equal to or greater than 200 mg/dL or a hemoglobin A1c level that is 6.5% or higher (e.g., Khokhar et al., 2017 Clin. Diabetes 35(3):133.)

Reduction in RA symptoms may be evidenced, for example by way of illustration and not limitation, as reduction of any one or more of fatigue, loss of appetite, low fever, swollen glands, weakness, swollen joints, joint pain, morning stiffness, warm, tender, or stiff joints when not used for as little as an hour, bilateral joint pain (fingers (but not the fingertips), wrists, elbows, shoulders, hips, knees, ankles, toes, jaw, and neck may be affected); loss of range of motion of affected joints, pleurisy, eye burning, eye itching, eye discharge, nodules under the skin, numbness, tingling, or burning in the hands and feet. Criteria for diagnosis and clinical monitoring of RA patients are well known to those skilled in the relevant art. See, e.g., Hochberg et al., Rheumatology, 2010 Mosby; Firestein et al., Textbook of Rheumatology, 2008 Saunders. Criteria for diagnosis and clinical monitoring of patients having RA and/or other autoimmune diseases are also well known to those skilled in the relevant art. See, e.g., Petrov, Autoimmune Disorders: Symptoms, Diagnosis and Treatment, 2011 Nova Biomedical Books; Mackay et al. (Eds.), The Autoimmune Diseases-Fourth Edition, 2006 Academic Press; Brenner (Ed.), Autoimmune Diseases: Symptoms, Diagnosis and Treatment, 2011 Nova Science Pub. Inc.

Standard techniques may be used for recombinant DNA, peptide and oligonucleotide synthesis, immunoassays and tissue culture and transformation (e.g., electroporation, or lipofection). Enzymatic reactions and purification techniques may be performed according to manufacturer's specifications or as commonly accomplished in the art or as described herein. These and related techniques and procedures may be generally performed according to conventional methods well known in the art and as described in various general and more specific references in microbiology, molecular biology, biochemistry, molecular genetics, cell biology, virology and immunology techniques that are cited and discussed throughout the present specification. See, e.g., Sambrook, et al., Molecular Cloning: A Laboratory Manual, 3d ed., Cold Spring Harbor Laboratory Press, Cold Spring Harbor, N.Y.; Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (TRL Press, Oxford Univ. Press USA, 1985); Current Protocols in Immunology (Edited by: John E. Coligan, Ada M. Kruisbeek, David H. Margulies, Ethan M. Shevach, Warren Strober 2001 John Wiley & Sons, NY, NY); Real-Time PCR: Current Technology and Applications, Edited by Julie Logan, Kirstin Edwards and Nick Saunders, 2009, Caister Academic Press, Norfolk, UK; Anand, Techniques for the Analysis of Complex Genomes, (Academic Press, New York, 1992); Guthrie and Fink, Guide to Yeast Genetics and Molecular Biology (Academic Press, New York, 1991); Oligonucleotide Synthesis (N. Gait, Ed., 1984); Nucleic Acid Hybridization (B. Hames & S. Higgins, Eds., 1985); Transcription and Translation (B. Hames & S. Higgins, Eds., 1984); Animal Cell Culture (R. Freshney, Ed., 1986); Perbal, A Practical Guide to Molecular Cloning (1984); Next-Generation Genome Sequencing (Janitz, 2008 Wiley-VCH); PCR Protocols (Methods in Molecular Biology) (Park, Ed., 3rd Edition, 2010 Humana Press); Immobilized Cells And Enzymes (IRL Press, 1986); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Harlow and Lane, Antibodies, (Cold Spring Harbor Laboratory Press, Cold Spring Harbor, N.Y., 1998); Immunochemical Methods In Cell And Molecular Biology (Mayer and Walker, eds., Academic Press, London, 1987); Handbook Of Experimental Immunology, Volumes I-IV (D. M. Weir and C C Blackwell, eds., 1986); Roitt, Essential Immunology, 6th Edition, (Blackwell Scientific Publications, Oxford, 1988); Embryonic Stem Cells: Methods and Protocols (Methods in Molecular Biology) (Kurstad Turksen, Ed., 2002); Embryonic Stem Cell Protocols: Volume I: Isolation and Characterization (Methods in Molecular Biology) (Kurstad Turksen, Ed., 2006); Embryonic Stem Cell Protocols: Volume II: Differentiation Models (Methods in Molecular Biology) (Kurstad Turksen, Ed., 2006); Human Embryonic Stem Cell Protocols (Methods in Molecular Biology) (Kursad Turksen Ed., 2006); Mesenchymal Stem Cells: Methods and Protocols (Methods in Molecular Biology) (Darwin J. Prockop, Donald G. Phinney, and Bruce A. Bunnell Eds., 2008); Hematopoietic Stem Cell Protocols (Methods in Molecular Medicine) (Christopher A. Klug, and Craig T. Jordan Eds., 2001); Hematopoietic Stem Cell Protocols (Methods in Molecular Biology) (Kevin D. Bunting Ed., 2008) Neural Stem Cells: Methods and Protocols (Methods in Molecular Biology) (Leslie P. Weiner Ed., 2008).

### Certain Gene Editing Embodiments

Any one of a number of gene- or genome- editing methods can used to accomplish editing of the TRAC locus and/or FOXP3 gene/locus. Non-limiting gene editing methods include zinc finger nuclease (ZFN)-mediated gene editing, transcription activator-like effector nuclease (TALEN)-mediated gene editing, meganuclease-mediated gene editing, transposon-mediated gene editing, serine integrase-mediated gene editing, lentivirus-mediated gene editing, RNA-guided nuclease (RGN)-mediated gene editing, CRISPR/Cas-mediated gene editing, homologous recombination-mediated gene editing, and combinations thereof. Some embodiments of the methods and compositions provided herein include systems for gene editing. Some embodiments include approaches using viral approaches, e.g., using lentivirus or AAV, or non-virus approaches, e.g., transposon-based methods. Transposon-based systems are described in Woodard L. E. et al. (2012) PLoS ONE 7(11): e42666; and Amberger M. et al., (2020) BioEssays 42: 20000136, which are each incorporated by reference in its entirety.

Gene-editing systems as described herein can make use of viral or non-viral vectors or cassettes, as well as nucleases that allow site-specific or locus-specific gene-editing, such as RNA-guided nucleases, CRISPR/Cas nucleases (e.g., Cpf1 or Cas9 nucleases), meganucleases, TALENs, or ZFNs., as described in paragraph [0039]. Certain RNA-guided nucleases useful with some embodiments provided herein are disclosed in U.S. Pat. No. 11,162,114, which is expressly incorporated by reference herein in its entirety. Non-limiting examples of CRISPR/Cas nucleases include SpCas9, SaCas9, CjCas9, xCas9, C2c1, Casl3a/C2c2, C2c3, Casl3b, Cpf1, and variants thereof. Certain features useful with some embodiments provided herein are disclosed in WO 2019/210057, which is expressly incorporated by reference in its entirety.

This application is based, at least in part, on the unexpected finding that split CISC function is dependent upon expression of the different components of the CISC system and/or location of these elements within a construct/cassette/polynucleotide (e.g., an HDR cassette). Accordingly, in some embodiments, one or more constructs for a gene-editing system as described herein comprises a promoter (e.g., an MND promoter) that results in a high expression of CISC component compared to promoters (e.g., EF1-alpha) that results in a lower expression of CISC components. In some embodiments, the promoter is selected from an MND, EF-1alpha, PJET, UBC, or CMV promoter. In some embodiments, the promoter is an MND promoter. In some embodiments, a first polynucleotide comprising a nucleic acid encoding a first CISC component is inserted into a first locus of a cell, and a second nucleic acid encoding a second CISC component is inserted into a second locus of cell that is different from the first locus. In some embodiments, one or both of the polynucleotides comprising nucleic acids encoding a first or second CISC component also comprises a nucleic acid encoding a third CISC component that is soluble and does not comprise an extracellular or transmembrane domain, and is capable of specifically binding to the CISC inducer molecule. In some embodiments, a first polynucleotide comprises an MND promoter that is operably linked to the nucleic acid that encodes the first CISC component. In some embodiments, a second polynucleotide comprises an MND promoter that is operably linked to the nucleic acid encoding the second CISC component. In some embodiments, the first CISC component comprises an FKBP extracellular domain, a transmembrane domain, and an IL2RB intracellular signaling domain, the second CISC component comprises an FRB extracellular domain, a transmembrane domain, and an IL2RG intracellular signaling domain, and the third CISC component comprises a soluble FRB domain. In some embodiments, the first CISC component comprises an FKBP extracellular domain, a transmembrane domain, and an IL2RG intracellular signaling domain, the second CISC component comprises an FRB extracellular domain, a transmembrane domain, and an IL2RB intracellular signaling domain, and the third CISC component comprises a soluble FRB domain. In some embodiments, the first polynucleotide comprising a nucleic acid encoding the first CISC component further comprises a nucleic acid encoding a TCR polypeptide or a portion thereof. In some embodiments, the first polynucleotide comprising a nucleic acid encoding the first CISC component further comprises a nucleic acid encoding a CAR polypeptide. In some embodiments, the first polynucleotide comprising a nucleic acid encoding the first CISC component comprises an MND promoter that, after insertion into the genome of a cell, becomes operably linked to an endogenous FOXP3 gene or a portion thereof, such that the MND promoter controls transcription of the endogenous FOXP3 gene. In some embodiments, the second polynucleotide comprising a nucleic acid encoding the second CISC component further comprises a nucleic acid encoding a TCR polypeptide or a portion thereof. In some embodiments, the second polynucleotide comprising a nucleic acid encoding the second CISC component further comprises a nucleic acid encoding a CAR polypeptide. In some embodiments, the second polynucleotide comprising a nucleic acid encoding the second CISC component comprises an MND promoter that, after insertion into the genome of a cell, becomes operably linked to an endogenous FOXP3 gene or a portion thereof, such that the MND promoter controls transcription of the endogenous FOXP3 gene. In some embodiments, provided herein is a method comprising contacting a cell with (i) a first polynucleotide comprising an MND promoter operably linked to a nucleic acid encoding (a) a first CISC component comprising an FRB extracellular domain, a transmembrane domain, and an IL2RB intracellular signaling domain, and (b) a nucleic acid encoding a third CISC component comprising a soluble FRB domain, wherein the MND promoter is inserted upstream of the first coding exon of the endogenous FOXP3 gene, and downstream of (e.g., 10 to 10,000 bp downstream from) a TSDR in the endogenous FOXP3 locus of the cell; and (ii) a second nucleic acid comprising an MND promoter operably linked to a nucleic acid encoding (a) a second CISC component comprising an FKBP extracellular domain, a transmembrane domain, and an IL2RG intracellular domain, (b) a nucleic acid encoding an exogenous TCRβ, and (c) a nucleic acid encoding an exogenous TRAV and TRAJ amino acid sequences, wherein the second nucleic acid is inserted into the TRAC locus upstream from nucleic acid encoding the TCRα constant region amino acid sequence, wherein the inserted MND promoter controls transcription of the exogenous TCRβ and a TCRα comprising the exogenous TRAV and TRAJ amino acid sequences and the endogenous TCRα amino acid sequences. In some embodiments, the method further comprises delivering to the cell a guide RNA targeting the TRAC locus, a guide RNA targeting the FOXP3 locus, and an RNA-guided nuclease or a nucleic acid encoding the RNA-guided endonuclease. In some embodiments, the method comprises delivering to the cell a first nuclease that cleaves a nucleotide sequence within the FOXP3 locus or a nucleic acid encoding the first nuclease, and a second nuclease that cleaves a nucleotide sequence within the TRAC locus or a nucleic acid encoding the second nuclease. It should be understood that any order of nucleic acids on a polynucleotide are contemplated herein. For example, a polynucleotide may have a nucleic acid encoding a TCR or portion thereof that is in close proximity to the promoter relative to the proximity of nucleic acid encoding a CISC component to the promoter. On the other hand, a nucleic acid encoding a CISC component could be in closer proximity to a promoter relative to a nucleic acid encoding a TCR or portion thereof.

In some embodiments, provided herein is a genetically modified cell comprising in its genome (i) a first polynucleotide comprising an MND promoter operably linked to a nucleic acid encoding (a) a first CISC component comprising an FRB extracellular domain, a transmembrane domain, and an IL2RB intracellular signaling domain, and (b) a third CISC component comprising a soluble FRB domain, wherein the MND promoter is inserted upstream of the first coding exon of the endogenous FOXP3 gene, and downstream of (e.g., 10 to 10,000 bp downstream from) a TSDR in the endogenous FOXP3 locus of the cell; and (ii) a second polynucleotide comprising an MND promoter operably linked to a nucleic acid encoding (a) a second CISC component comprising an FKBP extracellular domain, a transmembrane domain, and an IL2RG intracellular domain, (b) an exogenous TCRβ, and (c) an exogenous TRAV and TRAJ amino acid sequences, wherein the second nucleic acid is inserted into the TRAC locus upstream from nucleic acid encoding the TCRα constant region amino acid sequence, wherein the inserted MND promoter is controls transcription of the exogenous TCRβ and a TCRα comprising the exogenous TRAV and TRAJ amino acid sequences and the endogenous TCRα amino acid sequences.

In some embodiments, provided herein is a method of treating an autoimmune, allergic, and/or inflammatory disease in a subject, comprising providing to the subject a genetically modified cell comprising in its genome (i) a first polynucleotide comprising an MND promoter operably linked to a nucleic acid encoding (a) a first CISC component comprising an FRB extracellular domain, a transmembrane domain, and an IL2RB intracellular signaling domain, and (b) a third CISC component comprising a soluble FRB domain, wherein the MND promoter is inserted upstream of the first coding exon of the endogenous FOXP3 gene, and downstream of (e.g., 10 to 10,000 bp downstream from) a TSDR in the endogenous FOXP3 locus of the cell; and (ii) a second polynucleotide comprising an MND promoter operably linked to a nucleic acid encoding (a) a second CISC component comprising an FKBP extracellular domain, a transmembrane domain, and an IL2RG intracellular domain, (b) an exogenous TCRβ, and (c) an exogenous TRAV and TRAJ amino acid sequences, wherein the second polynucleotide is inserted into the TRAC locus upstream from nucleic acid encoding the TCRα constant region amino acid sequence, wherein the inserted MND promoter is controls transcription of the exogenous TCRβ and a TCRα comprising the exogenous TRAV and TRAJ amino acid sequences and the endogenous TCRα amino acid sequences.

### Method of Optimizing Level CISC Component Expression

Contemplated herein is a method of optimizing the expression of CISC components. In some embodiments, expression of CISC components is optimized by orienting nucleic acid(s) encoding them to be proximal to a promoter. In some embodiments, expression of CISC components is optimized by the choice of promoter used for encoding the CISC components. In some embodiments, expression of CISC components is optimized by using the same promoter to promote transcription of each CISC component. In some embodiments, each CISC component is expressed under the control of a constitutive promoter. In some embodiments, each CISC component is expressed under the control of a PGK, EF-1a, or MND promoter. In some embodiments, each CISC component is expressed under the control of an MND promoter. Some embodiments of the methods provided herein comprise (i) inserting a first donor template comprising an MND promoter operably linked to a nucleotide sequence encoding a first CSC component into a first locus; and (ii) inserting a second donor template comprising an MND promoter operably linked to a nucleotide sequence encoding a second CISC component into a second locus, wherein the MND promoter of the first and/or second donor template is operably linked to a nucleotide sequence encoding a third CISC component that is capable of binding to the CISC inducer molecule. In some embodiments, the first and second CISC components are expressed at a consistent level. In some embodiments, the first and second CISC components are expressed such that the abundance of the first CISC component is between 50% to 150%, 60% to 140%, 70% to 130%, 80% to 120%, 90% to 110%, or 95% to 105% of the abundance of the second CISC component. In some embodiments, the relative abundance of each CISC component is measured by comparing the number of RNA transcripts encoding each CISC component. In some embodiments, the relative abundance of each CISC component is measured by comparing the number of protein molecules of each CISC component.

Some embodiments include a gene editing chemical-inducible signaling complex (CISC) system comprising a first polynucleotide comprising a first promoter and a nucleic acid encoding a first CISC component comprising a first extracellular binding domain, a transmembrane domain, and a first signaling domain, wherein the first promoter is proximal to the nucleic acid encoding a first CISC component. Some embodiments also include a second polynucleotide comprising a second promoter and a nucleic acid encoding a second CISC component comprising a second extracellular binding domain, a transmembrane domain, and a second signaling domain. In some embodiments, the first CISC component and the second CISC component are configured such that when expressed in a cell, they are capable of dimerizing in the presence of a dimerizing agent, such as rapamycin or a rapalog to generate a signaling-competent CISC.

In some embodiments, a promoter is proximal to a nucleic acid encoding a CISC component if it is within a certain distance (e.g., within 500, 400, 300, 200, 100, 50, 10, 5 consecutive nucleotides) from the 3′ end of the promoter. In some embodiments, a promoter is proximal to a nucleic acid encoding a CISC component if there are no other elements (e.g., an antigen-specific moiety) between the promoter and the CISC component.

In some embodiments, a 3′ end of the first promoter is located from a 5′ end of the nucleic acid encoding a first CISC component within 500, 400, 300, 200, 100, 50, 10, 5 consecutive nucleotides, or within a range defined by any two of the foregoing numbers of consecutive nucleotides.

In some embodiments, the first polynucleotide is configured for integration into a first target locus of a genome, and the second polynucleotide is configured for integration into a second target locus of the genome. For example, a first polynucleotide can include sequences homologous to a first target locus, and/or a second polynucleotide can include sequences homologous to a second target locus. In some embodiments, the first target locus is different from the second target locus. In some embodiments, the first target locus is located on a chromosome different from the second target locus. In some embodiments, the first target locus is selected from a TRAC locus or a FOXP3 locus; and the second target locus is selected from a TRAC locus or a FOXP3 locus. In some such embodiments, the first target locus is different from the second target locus. In some embodiments, the first locus is any locus in the genome of a cell, and the second locus is any locus in the genome of the cell that does not overlap with the first locus. In some embodiments, the first and second locus are the same or overlap. In some embodiments, a locus comprises a nucleic acid sequence encoding a gene. In some embodiments, a locus comprises a nucleic acid comprising one or more exons encoding a gene, and one or more nucleotides between the one or more exons. In some embodiments, a locus comprises a nucleic acid comprising a one or more regulatory elements and a nucleic acid sequence encoding a gene. In some embodiments, a single gene in a genome comprises multiple loci. In some embodiments, multiple loci comprise multiple nucleic acid sequences that do not share common nucleotides. In some embodiments, multiple loci comprise multiple nucleic acid sequences, and no nucleotide belongs to more than one locus. In some embodiments, the first polynucleotide encodes a first CISC component, and the second polynucleotide encodes a second CISC component.

In some embodiments, the first extracellular binding domain comprises an FK506 binding protein (FKBP)-rapamycin binding (FRB) domain; and the second extracellular binding domain comprises an FKBP domain. In some embodiments, the first signaling domain comprises an IL-2 receptor subunit beta (IL2Rβ) domain or functional derivative thereof; and the second signaling domain comprises an IL-2 receptor subunit gamma (IL2Rβγ) domain or functional derivative thereof. In some embodiments, the IL2Rβ domain comprises a truncated IL2Rβ domain.

In some embodiments, the first and/or second promoter comprises a constitutive promoter. In some embodiments, the first and/or second promoter comprises a MND promoter.

In some embodiments, the system comprises a first vector comprising the first polynucleotide, and a second vector comprises the second polynucleotide. In some embodiments, the first vector and/or the second vector comprises a viral vector. In some embodiments, the first vector and/or the second vector comprises a lentiviral, an adenoviral, or an adeno-associated viral (AAV) vector.

In some embodiments, the first polynucleotide and/or the second polynucleotide comprises a nucleic acid encoding a naked FRB domain, wherein the nucleic acid encoding a naked FRB domain lacks a nucleic acid encoding an endoplasmic reticulum localization signal polypeptide.

In some embodiments, the first polynucleotide and/or the second polynucleotide comprises a nucleic acid encoding a payload. In some embodiments, the first polynucleotide and/or the second polynucleotide comprises a nucleic acid encoding a self-cleaving polypeptide, wherein the nucleic acid encoding a self-cleaving polypeptide is 5′ of the nucleic acid encoding a payload. In some embodiments, the self-cleaving polypeptide is selected from the group consisting of P2A, T2A, E2A, and F2A. In some embodiments, the payload comprises a T cell receptor (TCR), chimeric antigen receptor (CAR), or functional fragment thereof. In some embodiments, the TCR comprises the polypeptide sequence of any one of SEQ ID NOs 1377-1390.

In some embodiments, the first polynucleotide and/or the second polynucleotide is configured for integration into a target genomic locus by a recombination technique, such as homology directed repair (HDR) or by non-homologous end joining (NHEJ). Some embodiments also include a guide RNA (gRNA) and a DNA endonuclease. In some embodiments, the DNA endonuclease comprises a Cas9 endonuclease.

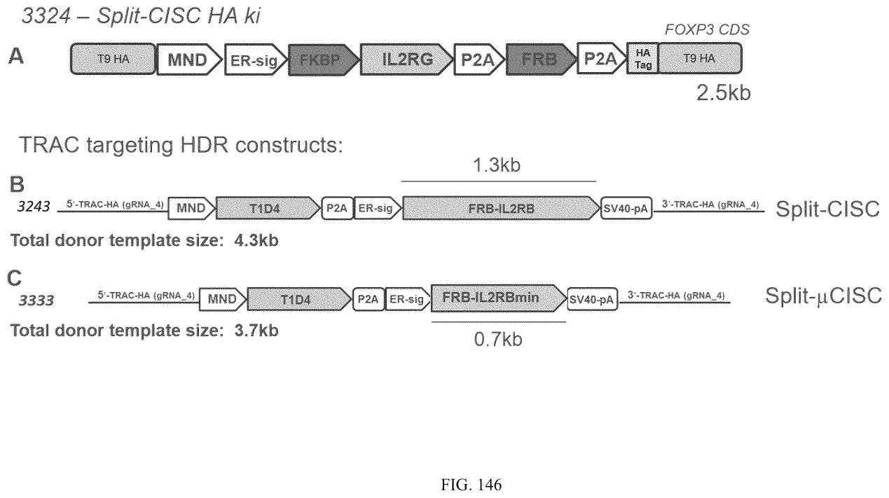

In some embodiments, the first nucleic acid of the FOXP3 knock-in construct is a FKBP-IL2RG, and the second nucleic acid is an FRB, wherein a dual editing strategy is used, and further wherein the FKBP-IL2RG component of the Split-CISC (AAV donor #3324) is association with a second AAV HDR donor targeting the TRAC locus and designed to express the T1D4 islet TCR in combination of the FRB-IL2RB component of the Split-CISC (FIG. 146, panel A).

In some embodiments, a polynucleotide is configured for integration into the TRAC locus to express an exogenous TCR or CAR and a CISC component under the control of an endogenous promoter (see e.g., FIGS. 54, 68, and 70). In some embodiments, the polynucleotide is integrated into the TRAC locus, wherein a nucleic acid sequence encoding TCRβ, a nucleic acid sequence encoding TCRα, and a nucleic acid sequence encoding a CISC component, optionally FRB-IL2Rβ, are inserted downstream of (e.g., 10 to 10,000 bp downstream from) an endogenous promoter. In some embodiments, the endogenous promoter is operably linked to an open reading frame encoding a polypeptide comprising 1) a TCRβ, 2) a TCRα, and 3) a CISC component, optionally a FRB-IL2Rβ polypeptide, wherein each of the three polypeptides are separated by a P2A self-cleavage motif. In some embodiments, the TCRα polypeptide and TCRβ polypeptides form a T1D4 islet-specific TCR.

In some embodiments, a polynucleotide is configured for integration into the TRAC locus to express an exogenous TCR and a CISC component under the control of an exogenous promoter (see e.g., FIG. 67). In some embodiments, the polynucleotide is integrated into the TRAC locus, wherein the polynucleotide comprises an exogenous promoter that is operably linked to 1) a nucleic acid sequence encoding TCRβ, 2) a nucleic acid sequence encoding TCRα, and 3) a nucleic acid sequence encoding a CISC component, optionally FRB-IL2Rβ. In some embodiments, the exogenous promoter is operably linked to an open reading frame encoding a polypeptide comprising 1) a TCRβ polypeptide, 2) a TCRα polypeptide, and 3) a CISC component, optionally a FRB-IL2Rβ polypeptide, wherein each of the three polypeptides are separated by a P2A self-cleavage motif. In some embodiments, the TCRα polypeptide and TCRβ polypeptides form a T1D4 islet-specific TCR. In some embodiments, the exogenous promoter is an MND promoter.

In some embodiments a polynucleotide is configured for integration into the TRAC locus to hijack the endogenous TRAC gene and express at least a portion of the endogenous TRAC gene under the control of an exogenous promoter (see e.g., FIG. 164). In some embodiments, the polynucleotide comprises an exogenous promoter that is operably linked to 1) a nucleic acid sequence encoding a CISC component, optionally FKBP-IL2Ry, 2) a nucleic acid sequence encoding a TCRβ polypeptide, and 3) a nucleic acid sequence encoding a portion of a TCRα polypeptide, such as a portion comprising one or more complementarity determining regions (CDRs) and/or framework regions that affect the specificity of the TCRα protein. In some embodiments the polynucleotide is inserted into the TRAC locus such that the nucleic acid sequence encoding the portion of a TCRα polypeptide is inserted in-frame with a portion of the endogenous TCRα coding sequence, and after integration the TRAC locus comprises a nucleic acid sequence encoding a full-length TCRα polypeptide. In some embodiments, integration of the polynucleotide results in the TRAC locus containing an exogenous promoter that is operably linked to a nucleic acid sequence encoding a polypeptide encoding 1) a CISC component, optionally FKBP-IL2Ry, 2) a TCRβ polypeptide, and 3) a TCRα polypeptide, wherein each of the three polypeptides are separated by a P2A self-cleavage motif. In some embodiments, the TCRα and TCRβ polypeptides form a T1D4 islet-specific TCR. In some embodiments, the exogenous promoter is an MND promoter.

In some embodiments, the first polypeptide of the TRAC-targeting HDR construct is a T1D4, and the second polypeptide is an FRB-IL2B (AA237-551; AAV #3243) (FIG. 146, panel B). In some embodiments, the first polypeptide of the TRAC-targeting HDR construct is T1D4, and the second polypeptide is an FRB-IL2Bmin polypeptide, which comprises a truncated intracellular IL-2 receptor beta signaling domain that retains key tyrosine residues (AA233-350; AAV donor #3333), referred to here as Split-μCISC (FIG. 146, panel C). Importantly, in these configurations, the FRB-IL2RB CISC component within the TRAC targeting construct lies downstream of two P2A elements from the MND promoter, potentially impacting its expression level.

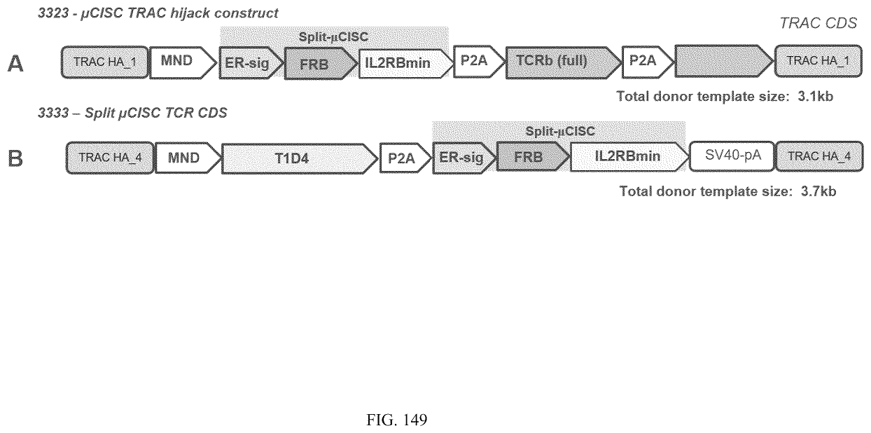

In some embodiments, a micro-CISC component of the TRAC-targeting HDR construct is proximal to an MND promoter (FIG. 149, upper). In some embodiments, a TCR (T1D4) component of the TRAC-targeting HDR construct is proximal to an MND promoter (FIG. 149, lower). By design, this approach re-orients the μCISC from previous constructs such that it is the first peptide expressed after the MND promoter. These embodiments serve as alternative strategy to introduce defined TCRs at the TRAC locus, wherein a TRAC hijack strategy is employed that knocks in the TCR-alpha variable regions in-frame to the endogenous TRAC gene, thereby reducing the total size of the repair template.

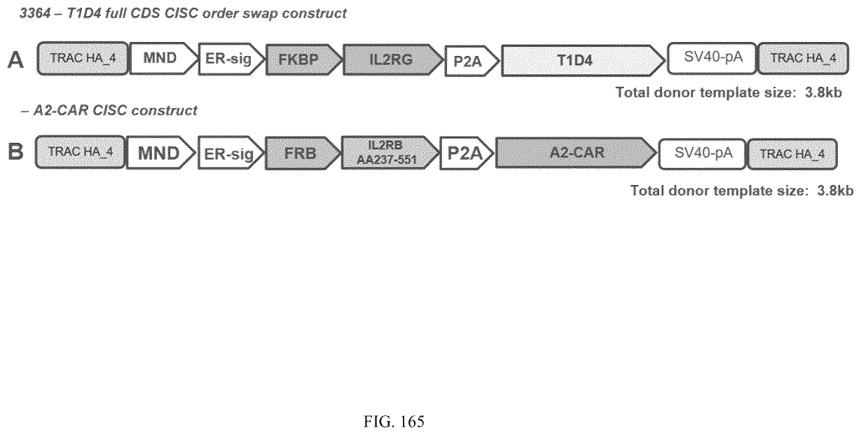

In some embodiments, the first polypeptide of the full CISC TRAC hijack construct (3354) is an FRB-IL2RB AA237-551, the second polypeptide is a full-length TCRb, and the third polypeptide is a TRAV/TRAJ (FIG. 164, panel A). In some embodiments, the first polypeptide of the full CISC component swap TRAC hijack construct (3363) is a an FKBP-IL2RG, the second polypeptide is a full-length TCRb, and the third polypeptide is a TRAV/TRAJ (FIG. 164, panel B). In some embodiments, the first polypeptide of the CISC component swap FOXP3 construct (3362) is an FRB-IL2RB AA237-551, and the second polypeptide is an FRB (FIG. 164, panel C). In some embodiments, the first polypeptide of the T1D4 full CDS CISC order swap construct (3364) comprising is an FKBP-IL2RG polypeptide, and the second polypeptide is a T1D4 (FIG. 165, panel A). In some embodiments, the first polypeptide of the A2-CAR CISC construct is an FRB-IL2RB AA237-551, and the second polypeptide is an A2-CAR (FIG. 165, panel B). The constructs disclosed herein are designed to optimize CISC component expression and tested for the generation of dual-edited antigen-specific, CISC expressing engTregs. As disclosed herein, the constructs are also evaluated for CISC functionality and antigen-specific engTreg function.

In some embodiments, the rapalog is selected from the group consisting of everolimus, CCI-779, C20-methallylrapamycin, C16-(S)-3-methylindolerapamycin, C16-iRap, C16-(S)-7-methylindolerapamycin, AP21967, C16-(S)Butylsulfonamidorapamycin, AP23050, sodium mycophenolic acid, benidipine hydrochloride, AP1903, and AP23573, and a metabolite or derivative thereof.

Some embodiments include a cell comprising any one of the foregoing systems. In some embodiments, the cell is a T cell, a precursor T cell, or a hematopoietic stem cell. In some embodiments, the cell is an NK-T cell (e.g., a FOXP3− NK-T cell or a FOXP3+ NK-T cell). In some embodiments, the cell is a regulatory B (Breg) cell (e.g., a FOXP3− B cell or a FOXP3+ B cell). In some embodiments, the cell is a CD4+ T cell (e.g., a FOXP3−CD4+ T cell or a FOXP3+CD4+ T cell) or a CD8+ T cell (e.g., a FOXP3−CD8+ T cell or a FOXP3+CD8+ T cell). In some embodiments, the cell is a CD25− T cell. In some embodiments, the cell is a regulatory T (Treg) cell. In some embodiments, a cell as provided herein is an engineered cell. In some embodiments, an engineered cell is a cell in which one or more genes/loci are manipulated or edited (e.g., to stabilize expression of one or more genes). In some embodiments, an engineered cell comprises editing of the Foxp3 gene/locus, e.g., by inserting a promoter (in some embodiments, downstream of (e.g., 10 to 10,000 bp downstream from) one or more regulatory elements like the TSDR, and/or upstream from the first coding exon). In some embodiments, the cell is a T regulatory type 1 (Tr1) cell. In some embodiments, the Treg cell is a FOXP3+ Treg cell. In some embodiments, the Treg cell expresses CTLA-4, LAG-3, CD25, CD39, neuropilin-1, galectin-1, and/or IL-2Ra on its surface. In some embodiments, a cell as provided herein is an engineered cell. In some embodiments, an engineered cell is a cell in which one or more genes/loci are manipulated or edited (e.g., to stabilize expression of one or more genes). In some embodiments, an engineered cell comprises editing of the Foxp3 gene/locus, e.g., by inserting a promoter (in some embodiments, downstream of (e.g., 10 to 10,000 bp downstream from) one or more regulatory elements like the TSDR, and/or upstream from the first coding exon). In some embodiments, the cell is ex vivo. In some embodiments, the cell is a human cell. In some embodiments, the cell is obtained from peripheral blood. In some embodiments, the cell is obtained from umbilical cord blood. In some embodiments, the cell is an allogeneic cell. In some embodiments, the cell is an autologous cell. Some embodiments include a FOXP3+ Treg cell comprising one or more nucleic acid sequences encoding a) a first transmembrane receptor polypeptide comprising a first extracellular ligand-binding domain, a transmembrane domain, and an IL-2Rβ intracellular signaling domain; b) a second transmembrane receptor polypeptide comprising a first extracellular ligand-binding domain, a transmembrane domain, and an IL-2Rγ intracellular signaling domain; and c) an intracellular FKBP-rapamycin-binding (FRB) polypeptide. In some embodiments, one of the first or second extracellular ligand-binding domains is an FK506-binding protein (FKBP) domain or a derivative thereof, and the other extracellular ligand-binding domain is an FKBP-rapamycin-binding (FRB) domain or a derivative thereof. In some embodiments, the rapalog is everolimus, CCI-779, C20-methallylrapamycin, C16-(S)-3-methylindolerapamycin, C16-iRap, C16-(S)Butylsulfonamidorapamycin, AP23050, C16-(S)-7-methylindolerapamycin, AP21967, sodium mycophenolic acid, benidipine hydrochloride, AP1903, or AP23573; a metabolite of rapamycin; or an IMID-class drug, optionally thalidomide, pomalidomide, lenalidomide. or an IMID-class drug analogue. In some embodiments, the ligand-binding domains of the first and second transmembrane receptors bind to rapamycin or a rapalog. In some embodiments, the first and second transmembrane receptors dimerize in the presence of the rapamycin or rapalog. In some embodiments, dimerization of the transmembrane receptors causes STAT5 phosphorylation and/or PI3K signal transduction, which promote survival and/or proliferation of the Treg cell. In some embodiments, the cell comprises a promoter inserted at the FOXP3 locus upstream of the first coding exon of FOXP3, which contains the start codon of the open reading frame encoding FOXP3. In some embodiments, the promoter is inserted downstream of (e.g., 10 to 10,000 bp downstream from) the TSDR. In some embodiments, the cell comprises an exogenous nucleic acid sequence encoding FOXP3 that is downstream from the inserted promoter, and the inserted promoter controls transcription of this exogenous FOXP3 coding sequence. In some embodiments, the inserted promoter is active, promoting transcription of mRNA encoding FOXP3, even under pro-inflammatory conditions. In some embodiments, the promoter is a constitutive promoter. In some embodiments, the promoter is an EF1a, PGK, or MND promoter. In some embodiments, the promoter is an MND promoter. In some embodiments, the cell comprises an exogenous nucleic acid sequence encoding a T cell receptor p protein or a portion thereof, a T cell receptor a protein or a portion thereof, or a chimeric antigen receptor or a portion thereof. In some embodiments, the T cell receptor or chimeric antigen that specifically recognizes an antigen associated with the pathogenesis of an autoimmune condition, an allergic condition, or an inflammatory condition. In some embodiments, the T cell receptor or chimeric antigen that specifically recognizes an antigen associated with the pathogenesis of an autoimmune disease (e.g., diabetes such as type-1 diabetes, primary biliary cholangitis), autoinflammatory disease (e.g., ARDS, stroke, and atherosclerotic cardiovascular disease), alloimmune disease (e.g., graft-versus-host disease, sold organ transplant, and immune mediated recurrent pregnancy loss), and/or allergic disease (e.g., asthma, drug hypersensitivity, and celiac disease). In some embodiments, a condition to be treated is a cancer. Wang et al. (J Intern Med. 2015 October;278(4):369-95) provide a review of autoimmune diseases, which review is incorporated herein by reference. In some embodiments (i) the autoimmune condition is selected from type 1 diabetes mellitus, multiple sclerosis, systemic lupus erythematosus, myasthenia gravis, rheumatoid arthritis, early onset rheumatoid arthritis, ankylosing spondylitis, immune-mediated pregnancy loss, immune-mediated recurrent pregnancy loss, dermatomyositis psoriatic arthritis, Crohn's disease, inflammatory bowel disease (IBD), ulcerative colitis, bullous pemphigoid, pemphigus vulgaris, autoimmune hepatitis, psoriasis, Sjogren's syndrome, or celiac disease; (ii) the allergic condition is selected from allergic asthma, steroid-resistant asthma, atopic dermatitis, celiac disease, pollen allergy, food allergy, drug hypersensitivity, or contact dermatitis; and (iii) the inflammatory condition is selected from pancreatic islet cell transplantation, asthma, hepatitis, traumatic brain injury, primary sclerosing cholangitis, primary biliary cholangitis, polymyositis, stroke, Still's disease, acute respiratory distress syndrome, uveitis inflammatory bowel disease (IBD), ulcerative colitis, graft-versus-host disease (GVHD), tolerance induction for transplantation, transplant rejection, or sepsis.

Some embodiments include a cell comprising one or more nucleic acid sequences encoding a) a first transmembrane receptor polypeptide comprising a first extracellular ligand-binding domain, a transmembrane domain, and an IL-2Rβ intracellular signaling domain; b) a second transmembrane receptor polypeptide comprising a first extracellular ligand-binding domain, a transmembrane domain, and an IL-2Rγ intracellular signaling domain; and c) an intracellular FKBP-rapamycin-binding (FRB) polypeptide. In some embodiments, one of the first or second extracellular ligand-binding domains is an FK506-binding protein (FKBP) domain or a derivative thereof, and the other extracellular ligand-binding domain is an FKBP-rapamycin-binding (FRB) domain or a derivative thereof. In some embodiments, the rapalog is everolimus, CCI-779, C20-methallylrapamycin, C16-(S)-3-methylindolerapamycin, C16-iRap, C16-(S)Butylsulfonamidorapamycin, AP23050, C16-(S)-7-methylindolerapamycin, AP21967, sodium mycophenolic acid, benidipine hydrochloride, AP1903, or AP23573; a metabolite of rapamycin; or an IMID-class drug, optionally thalidomide, pomalidomide, lenalidomide. or an IMID-class drug analogue. In some embodiments, the ligand-binding domains of the first and second transmembrane receptors bind to rapamycin or a rapalog. In some embodiments, the first and second transmembrane receptors dimerize in the presence of the rapamycin or rapalog. In some embodiments, dimerization of the transmembrane receptors causes STAT5 phosphorylation and/or PI3K signal transduction, which promote survival and/or proliferation of the Treg cell. In some embodiments, the cell comprises an exogenous nucleic acid sequence encoding a T cell receptor p protein or a portion thereof, a T cell receptor a protein or a portion thereof, or a chimeric antigen receptor or a portion thereof. In some embodiments, the cell comprises an exogenous nucleic acid sequence encoding a T cell receptor R protein or a portion thereof, a T cell receptor a protein or a portion thereof, or a chimeric antigen receptor or a portion thereof. In some embodiments, the T cell receptor or chimeric antigen that specifically recognizes an antigen associated with the pathogenesis of an autoimmune condition, an allergic condition, or an inflammatory condition. In some embodiments (i) the autoimmune condition is selected from type 1 diabetes mellitus, multiple sclerosis, systemic lupus erythematosus, myasthenia gravis, rheumatoid arthritis, early onset rheumatoid arthritis, ankylosing spondylitis, immune-mediated pregnancy loss, immune-mediated recurrent pregnancy loss, dermatomyositis psoriatic arthritis, Crohn's disease, inflammatory bowel disease (IBD), ulcerative colitis, bullous pemphigoid, pemphigus vulgaris, autoimmune hepatitis, psoriasis, Sjogren's syndrome, or celiac disease; (ii) the allergic condition is selected from allergic asthma, steroid-resistant asthma, atopic dermatitis, celiac disease, pollen allergy, food allergy, drug hypersensitivity, or contact dermatitis; and (iii) the inflammatory condition is selected from pancreatic islet cell transplantation, asthma, hepatitis, traumatic brain injury, primary sclerosing cholangitis, primary biliary cholangitis, polymyositis, stroke, Still's disease, acute respiratory distress syndrome, uveitis inflammatory bowel disease (IBD), ulcerative colitis, graft-versus-host disease (GVHD), tolerance induction for transplantation, transplant rejection, or sepsis. In some embodiments, the inflammatory condition is primary biliary cholangitis. In some embodiments, the inflammatory condition is primary sclerosing cholangitis. In some embodiments, the inflammatory condition is autoimmune hepatitis. In some embodiments, the autoimmune condition is type 1 diabetes. In some embodiments, the inflammatory condition is islet cell transplantation. In some embodiments, the inflammatory condition is transplant rejection. In some embodiments, the autoimmune condition is multiple sclerosis. In some embodiments, the inflammatory condition is inflammatory bowel disease. In some embodiments, the inflammatory condition is acute respiratory distress syndrome. In some embodiments, the inflammatory condition is stroke. In some embodiments, the inflammatory condition is graft-versus-host disease.

Some embodiments include a pharmaceutical composition comprising any one of the foregoing cells and a pharmaceutically acceptable excipient.

Some embodiments include a method of editing a cell, comprising obtaining any one of the foregoing systems; introducing the first polynucleotide and the second polynucleotide into a cell to obtain a transduced cell; and culturing the transduced cell. Some embodiments also include contacting the transduced cell with the rapamycin or rapalog. In some embodiments, the rapamycin or rapalog is present in a concentration of 0.001 nM to 10 μM, or at a concentration between 0.001 nM and 10 μM, when contacting the cell. In some embodiments, the concentration is an amount from 0.001 nM to 0.01 nM, 0.01 nM to 0.1 nM, 0.1 nM to 1.0 nM, 1.0 nM to 10 nM, 10 nM to 100 nM, 100 nM to 1 μM, or 1 μM to 10 μM.

Certain sequences, which can be incorporated into one or more embodiments provided herein, are listed in TABLE 1. It should be understood that polynucleotides and polypeptides as contemplated herein may be without any tags (e.g., HA tags) that may be comprised within any sequences exemplified in Table 1. Further, Table 1 provides examples of sequences for nucleic acids encoding an FRB domain and amino acids of proteins comprising FRB domains. It should be understood that any of nucleic acids encoding either a naked FRB or an FRB domain as part of a first or second CISC component are configured so that the encoded protein, and any of the proteins provided herein, may (i) comprise a T75L mutation (see e.g., constructs 3312, 3323, 3333, 3354, or 3362; and respectively SEQ ID NOs: 1445, 1444, 1443, 1446, or 1447) in some embodiments; or (ii) not comprise a T75L mutation (see e.g., SEQ ID NOs: 1450, 1451, or 1452) in some embodiments. Table 1 also provides examples of sequences for nucleic acids encoding IL2Rβ signaling domain as part of a first of second CISC component, and amino acids of proteins comprising IL2Rβ signaling domains. Some versions of such sequences are truncated, and other versions are not. It is to be understood that any of nucleic acids encoding a IL2Rβ signaling domain as provided herein may (i) comprise or encode a truncated version of IL2Rβ signaling domain (see e.g., constructs 3323, 3354, or 3333; or SEQ ID NOs: 1444, 1446, or 1443) in some embodiments, or (ii) comprise or encode an untruncated version of IL2Rβ signaling domain (see e.g., constructs 3312, 3354, or 3362; or SEQ ID NOs: 1445, 1446, 1447, 1451, or 1455).

TABLE 1

| Feature | |
|---|---|
| (SEQ ID | |
| NO) | Sequence |
| IL2Rγ- | MPLGLLWLGLALLGALHAQAGVQVETISPGDGRTFPKRGQTCVVHYTGMLEDGKK |
| CISC | FDSSRDRNKPFKFMLGKQEVIRGWEEGVAQMSVGQRAKLTISPDYAYGATGHPGI |
| polypeptide | IPPHATLVFDVELLKLGEGSNTSKENPFLFALEAVVISVGSMGLIISLLCVYFWL |
| SEQ ID | ERTMPRIPTLKNLEDLVTEYHGNFSAWSGVSKGLAESLQPDYSERLCLVSEIPPK |
| NO: 1437 | GGALGEGPGASPCNQHSPYWAPPCYTLKPET |
| naked FRB | MEMWHEGLEEASRLYFGERNVKGMFEVLEPLHAMMERGPQTLKETSFNQAYGRDL |
| wild-type | MEAQEWCRKYMKSGNVKDLTQAWDLYYHVFRRISK |
| polypeptide | |
| SEQ ID | |
| NO: 1438 | |
| naked FRB | MEMWHEGLEEASRLYFGERNVKGMFEVLEPLHAMMERGPQTLKETSFNQAYGRDL |
| mutant | MEAQEWCRKYMKSGNVKDLLQAWDLYYHVFRRISK |
| polypeptide | |
| SEQ ID | |
| NO: 1439 | |
| FRB-CISC | MALPVTALLLPLALLLHAARPILWHEMWHEGLEEASRLYFGERNVKGMFEVLEPL |
| having a | HAMMERGPQTLKETSFNQAYGRDLMEAQEWCRKYMKSGNVKDLLQAWDLYYHVFR |
| truncated | RISKPAALGKDTIPWLGHLLVGLSGAFGFIILVYLLINCRNTGPWLKKVLKCNTP |
| intracellular | DPSKFFSQLSSEHGGDVQKWLSSPFPSSSFSPGGLAPEISPLEVLERDKVTQLLL |
| IL2Rβ | QQDKVPEPASLSLNTDAYLSLQELQ |
| domain | |
| SEQ ID | |
| NO: 1440 | |
| truncated | PAALGKDTIPWLGHLLVGLSGAFGFIILVYLLINCRNTGPWLKKVLKCNTPDPSK |
| IL2Rβ | FFSQLSSEHGGDVQKWLSSPFPSSSFSPGGLAPEISPLEVLERDKVTQLLLQQDK |
| SEQ ID | VPEPASLSLNTDAYLSLQELQ |
| NO: 1441 | |
| Construct | GTAGAAAAGATCAAAGGATCTTCTTGAGATCCTTTTTTTCTGCGCGTAATCTGCT |
| 3324 | GCTTGCAAACAAAAAAACCACCGCTACCAGCGGTGGTTTGTTTGCCGGATCAAGA |
| SEQ ID | GCTACCAACTCTTTTTCCGAAGGTAACTGGCTTCAGCAGAGCGCAGATACCAAAT |
| NO: 1442 | ACTGTCCTTCTAGTGTAGCCGTAGTTAGGCCACCACTTCAAGAACTCTGTAGCAC |
| | CGCCTACATACCTCGCTCTGCTAATCCTGTTACCAGTGGCTGCTGCCAGTGGCGA |
| | TAAGTCGTGTCTTACCGGGTTGGACTCAAGACGATAGTTACCGGATAAGGCGCAG |
| | CGGTCGGGCTGAACGGGGGGTTCGTGCACACAGCCCAGCTTGGAGCGAACGACCT |
| | ACACCGAACTGAGATACCTACAGCGTGAGCTATGAGAAAGCGCCACGCTTCCCGA |
| | AGGGAGAAAGGCGGACAGGTATCCGGTAAGCGGCAGGGTCGGAACAGGAGAGCGC |
| | ACGAGGGAGCTTCCAGGGGGAAACGCCTGGTATCTTTATAGTCCTGTCGGGTTTC |
| | GCCACCTCTGACTTGAGCGTCGATTTTTGTGATGCTCGTCAGGGGGGCGGAGCCT |
| | ATGGAAAAACGCCAGCAACGCGGCCTTTTTACGGTTCCTGGCCTTTTGCTGGCCT |
| | TTTGCTCACATGTTCTTTCCTGCGTTATCCCCTGATTCTGTGGATAACCGTATTA |
| | CCGCCTTTGAGTGAGCTGATACCGCTCGCCGCAGCCGAACGACCGAGCGCAGCGA |
| | GTCAGTGAGCGAGGAAGCGGAAGAGCGCCCAATACGCAAACCGCCTCTCCCCGCG |
| | CGTTGGCCGATTCATTAATGCAGCTGCGCGCTCGCTCGCTCACTGAGGCCGCCCG |
| | GGCAAAGCCCGGGCGTCGGGCGACCTTTGGTCGCCCGGCCTCAGTGAGCGAGCGA |
| | GCGCGCAGAGAGGGAGTGGCCAACTCCATCACTAGGGGTTCCTTGTAGTTAATGA |
| | TTAACCCGCCATGCTACTTATCTACGTAGCGGCCGCAGCCTGTGCAGGGTGCAGG |
| | GAGGGCTAGAGGCCTGAGGCTTGAAACAGCTCTCAAGTGGAGGGGGAAACAACCA |
| | TTGCCCTCATAGAGGACACATCCACACCAGGGCTGTGCTAGCGTGGGCAGGCAAG |
| | CCAGGTGCTGGACCTCTGCACGTGGGGCATGTGTGGGTATGTACATGTACCTGTG |
| | TTCTTGGTGTGTGTGTGTGTGTGTGTGTGTGTGTGTGTCTAGAGCTGGGGTGCAA |
| | CTATGGGGCCCCTCGGGACATGTCCCAGCCAATGCCTGCTTTGACCAGAGGAGTG |
| | TCCACGTGGCTCAGGTGGTCGAGTATCTCATACCGCCCTAGCACACGTGTGACTC |
| | CTTTCCCCTATTGTCTACGCAGCCTGCCCTTGGACAAGGACCCGATGCCCAACCC |
| | CAGGCCTGGCAAGCCCTCGGCCCCTTCCTTGGCCCTTGGCCCATCCCCACGCGTA |
| | GGAACAGAGAAACAGGAGAATATGGGCCAAACAGGATATCTGTGGTAAGCAGTTC |
| | CTGCCCCGGCTCAGGGCCAAGAACAGTTGGAACAGCAGAATATGGGCCAAACAGG |
| | ATATCTGTGGTAAGCAGTTCCTGCCCCGGCTCAGGGCCAAGAACAGATGGTCCCC |
| | AGATGCGGTCCCGCCCTCAGCAGTTTCTAGAGAACCATCAGATGTTTCCAGGGTG |
| | CCCCAAGGACCTGAAATGACCCTGTGCCTTATTTGAACTAACCAATCAGTTCGCT |
| | TCTCGCTTCTGTTCGCGCGCTTCTGCTCCCCGAGCTCTATATAAGCAGAGCTCGT |
| | TTAGTGAACCGTCAGATCGCCTGGAGACGCCATCCACGCTGTTTTGACTTCCATA |
| | GAAGGATCTCGAGGCCACCATGCCACTGGGACTGCTGTGGCTTGGACTGGCCCTT |
| | CTTGGAGCACTGCATGCTCAGGCTGGCGTGCAGGTCGAGACAATTAGTCCTGGCG |
| | ACGGCCGGACCTTTCCTAAGCGAGGACAGACATGCGTGGTGCACTACACCGGCAT |
| | GCTGGAAGATGGCAAGAAGTTCGACAGCAGCCGGGACAGAAACAAGCCTTTCAAG |
| | TTCATGCTGGGCAAGCAAGAAGTGATCAGAGGCTGGGAAGAGGGCGTCGCCCAGA |
| | TGTCTGTTGGACAGAGAGCCAAGCTGACAATCAGCCCCGATTACGCCTATGGCGC |
| | CACAGGACACCCTGGGATCATTCCTCCACATGCCACACTGGTGTTCGATGTGGAA |
| | CTGCTGAAGCTCGGCGAAGGCAGCAATACCAGCAAAGAGAACCCCTTCCTGTTCG |
| | CCCTGGAAGCCGTGGTTATCAGCGTGGGATCTATGGGCCTGATCATCTCCCTGCT |
| | GTGCGTGTACTTCTGGCTGGAACGGACCATGCCTCGGATCCCCACACTGAAGAAT |
| | CTGGAAGATCTGGTCACCGAGTACCACGGCAATTTCAGCGCTTGGAGTGGCGTGT |
| | CCAAAGGCCTGGCTGAAAGCCTGCAGCCTGACTACTCTGAGAGACTGTGCCTGGT |
| | GTCTGAGATCCCTCCTAAAGGCGGCGCTCTCGGAGAAGGACCAGGCGCTTCTCCA |
| | TGCAATCAGCACAGCCCTTATTGGGCCCCTCCTTGCTACACCCTGAAGCCTGAAA |
| | CTGGATCCGGCGCCACCAACTTTAGTCTGCTCAAGCAAGCCGGGGACGTCGAGGA |
| | AAATCCTGGGCCAGAAATGTGGCACGAAGGACTCGAGGAAGCCAGTCGGCTGTAT |
| | TTTGGCGAGCGGAATGTGAAAGGGATGTTTGAAGTGCTCGAGCCTCTCCACGCTA |
| | TGATGGAACGGGGACCCCAGACTCTCAAAGAAACCAGCTTTAATCAGGCTTACGG |
| | ACGCGACCTCATGGAAGCTCAAGAATGGTGTAGAAAGTATATGAAGAGTGGCAAC |
| | GTGAAAGATCTGCTGCAAGCCTGGGATCTCTATTATCACGTGTTCAGACGCATCA |
| | GCAAAGGCAGCGGCGCCACAAATTTCTCCCTGCTGAAACAGGCCGGCGACGTGGA |
| | AGAGAATCCCGGACCTTATCCATACGATGTCCCAGATTATGCGCCCAATCCTAGA |
| | CCTGGCAAGCCCAGCGCTCCTTCTCTTGCTCTTGGACCTTCTCCTGGTGCCTCGC |
| | CCAGCTGGAGGGCTGCACCCAAAGCCTCAGACCTGCTGGGGGCCCGGGGCCCAGG |
| | GGGAACCTTCCAGGGCCGAGATCTTCGAGGCGGGGCCCATGCCTCCTCTTCTTCC |
| | TTGAACCCCATGCCACCATCGCAGCTGCAGGTGAGGCCCTGGGCCCAGGATGGGG |
| | CAGGCAGGGTGGGGTACCTGGACCTACAGGTGCCGACCTTTACTGTGGCACTGGG |
| | CGGGAGGGGGGCTGGCTGGGGCACAGGAAGTGGTTTCTGGGTCCCAGGCAAGTCT |
| | GTGACTTATGCAGATGTTGCAGGGCCAAGAAAATCCCCACCTGCCAGGCCTCAGA |
| | GATTGGAGGCTCTCCCCGACCTCCCAATCCCTGTCTCAGGAGAGGAGGAGGCCGT |
| | ATTGTAGTCCCATGAGCATAGCTATGTGTCCCCATCCCCATGTGACAAGAGAAGA |
| | GGAGGATCCTACGTAGATAAGTAGCATGGCGGGTTAATCATTAACTACAAGGAAC |
| | CCCTAGTGATGGAGTTGGCCACTCCCTCTCTGCGCGCTCGCTCGCTCACTGAGGC |
| | CGGGCGACCAAAGGTCGCCCGACGCCCGGGCTTTGCCCGGGCGGCCTCAGTGAGC |
| | GAGCGAGCGCGCCAGCTGGCGTAATAGCGAAGAGGCCCGCACCGATCGCCCTTCC |
| | CAACAGTTGCGCAGCCTGAATGGCGAATGGCGATTCCGTTGCAATGGCTGGCGGT |
| | AATATTGTTCTGGATATTACCAGCAAGGCCGATAGTTTGAGTTCTTCTACTCAGG |
| | CAAGTGATGTTATTACTAATCAAAGAAGTATTGCGACAACGGTTAATTTGCGTGA |
| | TGGACAGACTCTTTTACTCGGTGGCCTCACTGATTATAAAAACACTTCTCAGGAT |
| | TCTGGCGTACCGTTCCTGTCTAAAATCCCTTTAATCGGCCTCCTGTTTAGCTCCC |
| | GCTCTGATTCTAACGAGGAAAGCACGTTATACGTGCTCGTCAAAGCAACCATAGT |
| | ACGCGCCCTGTAGCGGCGCATTAAGCGCGGCGGGTGTGGTGGTTACGCGCAGCGT |
| | GACCGCTACACTTGCCAGCGCCCTAGCGCCCGCTCCTTTCGCTTTCTTCCCTTCC |
| | TTTCTCGCCACGTTCGCCGGCTTTCCCCGTCAAGCTCTAAATCGGGGGCTCCCTT |
| | TAGGGTTCCGATTTAGTGCTTTACGGCACCTCGACCCCAAAAAACTTGATTAGGG |
| | TGATGGTTCACGTAGTGGGCCATCGCCCTGATAGACGGTTTTTCGCCCTTTGACG |
| | TTGGAGTCCACGTTCTTTAATAGTGGACTCTTGTTCCAAACTGGAACAACACTCA |
| | ACCCTATCTCGGTCTATTCTTTTGATTTATAAGGGATTTTGCCGATTTCGGCCTA |
| | TTGGTTAAAAAATGAGCTGATTTAACAAAAATTTAACGCGAATTTTAACAAAATA |
| | TTAACGTTTACAATTTAAATATTTGCTTATACAATCTTCCTGTTTTTGGGGCTTT |
| | TCTGATTATCAACCGGGGTACATATGATTGACATGCTAGTTTTACGATTACCGTT |
| | CATCGATTCTCTTGTTTGCTCCAGACTCTCAGGCAATGACCTGATAGCCTTTGTA |
| | GAGACCTCTCAAAAATAGCTACCCTCTCCGGCATGAATTTATCAGCTAGAACGGT |
| | TGAATATCATATTGATGGTGATTTGACTGTCTCCGGCCTTTCTCACCCGTTTGAA |
| | TCTTTACCTACACATTACTCAGGCATTGCATTTAAAATATATGAGGGTTCTAAAA |
| | ATTTTTATCCTTGCGTTGAAATAAAGGCTTCTCCCGCAAAAGTATTACAGGGTCA |
| | TAATGTTTTTGGTACAACCGATTTAGCTTTATGCTCTGAGGCTTTATTGCTTAAT |
| | TTTGCTAATTCTTTGCCTTGCCTGTATGATTTATTGGATGTTGGAATCGCCTGAT |
| | GCGGTATTTTCTCCTTACGCATCTGTGCGGTATTTCACACCGCATATGGTGCACT |
| | CTCAGTACAATCTGCTCTGATGCCGCATAGTTAAGCCAGCCCCGACACCCGCCAA |
| | CACCCGCTGACGCGCCCTGACGGGCTTGTCTGCTCCCGGCATCCGCTTACAGACA |
| | AGCTGTGACCGTCTCCGGGAGCTGCATGTGTCAGAGGTTTTCACCGTCATCACCG |
| | AAACGCGCGAGACGAAAGGGCCTCGTGATACGCCTATTTTTATAGGTTAATGTCA |
| | TGATAATAATGGTTTCTTAGACGTCAGGTGGCACTTTTCGGGGAAATGTGCGCGG |
| | AACCCCTATTTGTTTATTTTTCTAAATACATTCAAATATGTATCCGCTCATGAGA |
| | CAATAACCCTGATAAATGCTTCAATAATATTGAAAAAGGAAGAGTATGAGTATTC |
| | AACATTTCCGTGTCGCCCTTATTCCCTTTTTTGCGGCATTTTGCCTTCCTGTTTT |
| | TGCTCACCCAGAAACGCTGGTGAAAGTAAAAGATGCTGAAGATCAGTTGGGTGCA |
| | CGAGTGGGTTACATCGAACTGGATCTCAACAGCGGTAAGATCCTTGAGAGTTTTC |
| | GCCCCGAAGAACGTTTTCCAATGATGAGCACTTTTAAAGTTCTGCTATGTGGCGC |
| | GGTATTATCCCGTATTGACGCCGGGCAAGAGCAACTCGGTCGCCGCATACACTAT |
| | TCTCAGAATGACTTGGTTGAGTACTCACCAGTCACAGAAAAGCATCTTACGGATG |
| | GCATGACAGTAAGAGAATTATGCAGTGCTGCCATAACCATGAGTGATAACACTGC |
| | GGCCAACTTACTTCTGACAACGATCGGAGGACCGAAGGAGCTAACCGCTTTTTTG |
| | CACAACATGGGGGATCATGTAACTCGCCTTGATCGTTGGGAACCGGAGCTGAATG |
| | AAGCCATACCAAACGACGAGCGTGACACCACGATGCCTGTAGCAATGGCAACAAC |
| | GTTGCGCAAACTATTAACTGGCGAACTACTTACTCTAGCTTCCCGGCAACAATTA |
| | ATAGACTGGATGGAGGCGGATAAAGTTGCAGGACCACTTCTGCGCTCGGCCCTTC |
| | CGGCTGGCTGGTTTATTGCTGATAAATCTGGAGCCGGTGAGCGTGGGTCTCGCGG |
| | TATCATTGCAGCACTGGGGCCAGATGGTAAGCCCTCCCGTATCGTAGTTATCTAC |
| | ACGACGGGGAGTCAGGCAACTATGGATGAACGAAATAGACAGATCGCTGAGATAG |
| | GTGCCTCACTGATTAAGCATTGGTAACTGTCAGACCAAGTTTACTCATATATACT |
| | TTAGATTGATTTAAAACTTCATTTTTAATTTAAAAGGATCTAGGTGAAGATCCTT |
| | TTTGATAATCTCATGACCAAAATCCCTTAACGTGAGTTTTCGTTCCACTGAGCGT |
| | CAGACCCC |
| Construct | GTAGAAAAGATCAAAGGATCTTCTTGAGATCCTTTTTTTCTGCGCGTAATCTGCT |
| 3333 | GCTTGCAAACAAAAAAACCACCGCTACCAGCGGTGGTTTGTTTGCCGGATCAAGA |
| SEQ ID | GCTACCAACTCTTTTTCCGAAGGTAACTGGCTTCAGCAGAGCGCAGATACCAAAT |
| NO: 1443 | ACTGTCCTTCTAGTGTAGCCGTAGTTAGGCCACCACTTCAAGAACTCTGTAGCAC |
| | CGCCTACATACCTCGCTCTGCTAATCCTGTTACCAGTGGCTGCTGCCAGTGGCGA |
| | TAAGTCGTGTCTTACCGGGTTGGACTCAAGACGATAGTTACCGGATAAGGCGCAG |
| | CGGTCGGGCTGAACGGGGGGTTCGTGCACACAGCCCAGCTTGGAGCGAACGACCT |
| | ACACCGAACTGAGATACCTACAGCGTGAGCTATGAGAAAGCGCCACGCTTCCCGA |
| | AGGGAGAAAGGCGGACAGGTATCCGGTAAGCGGCAGGGTCGGAACAGGAGAGCGC |
| | ACGAGGGAGCTTCCAGGGGGAAACGCCTGGTATCTTTATAGTCCTGTCGGGTTTC |
| | GCCACCTCTGACTTGAGCGTCGATTTTTGTGATGCTCGTCAGGGGGGCGGAGCCT |
| | ATGGAAAAACGCCAGCAACGCGGCCTTTTTACGGTTCCTGGCCTTTTGCTGGCCT |
| | TTTGCTCACATGTTCTTTCCTGCGTTATCCCCTGATTCTGTGGATAACCGTATTA |
| | CCGCCTTTGAGTGAGCTGATACCGCTCGCCGCAGCCGAACGACCGAGCGCAGCGA |
| | GTCAGTGAGCGAGGAAGCGGAAGAGCGCCCAATACGCAAACCGCCTCTCCCCGCG |
| | CGTTGGCCGATTCATTAATGCAGCTGCGCGCTCGCTCGCTCACTGAGGCCGCCCG |
| | GGCAAAGCCCGGGCGTCGGGCGACCTTTGGTCGCCCGGCCTCAGTGAGCGAGCGA |
| | GCGCGCAGAGAGGGAGTGGCCAACTCCATCACTAGGGGTTCCTTGTAGTTAATGA |
| | TTAACCCGCCATGCTACTTATCTACGTAGCGGCCGCACTGAAATCATGGCCTCTT |
| | GGCCAAGATTGATAGCTTGTGCCTGTCCCTGAGTCCCAGTCCATCACGAGCAGCT |
| | GGTTTCTAAGATGCTATTTCCCGTATAAAGCATGAGACCGTGACTTGCCAGCCCC |
| | ACAGAGCCCCGCCCTTGTCCATCACTGGCATCTGGACTCCAGCCTGGGTTGGGGC |
| | AAAGAGGGAAATGAGATCATGTCCTAACCCTGATCCTCTTGTCCCACAGATATCC |
| | AGAACCCTGACCCTGCCGTGTACCAGCTGAGAGACTCTAAATCCAGTGACAAGTC |
| | TGTCTGCCTATATTAAGGAACAGAGAAACAGGAGAATATGGGCCAAACAGGATAT |
| | CTGTGGTAAGCAGTTCCTGCCCCGGCTCAGGGCCAAGAACAGTTGGAACAGCAGA |
| | ATATGGGCCAAACAGGATATCTGTGGTAAGCAGTTCCTGCCCCGGCTCAGGGCCA |
| | AGAACAGATGGTCCCCAGATGCGGTCCCGCCCTCAGCAGTTTCTAGAGAACCATC |
| | AGATGTTTCCAGGGTGCCCCAAGGACCTGAAATGACCCTGTGCCTTATTTGAACT |
| | AACCAATCAGTTCGCTTCTCGCTTCTGTTCGCGCGCTTCTGCTCCCCGAGCTCTA |
| | TATAAGCAGAGCTCGTTTAGTGAACCGTCAGATCGCCTGGAGACGCCATCCACGC |
| | TGTTTTGACTTCCATAGAAGGATCTCGAGTAGCATGGGATCAAGACTGCTGTGCT |
| | GGGTGCTGCTGTGCCTGCTGGGAGCCGGACCTGTGAAAGCCGGGGTGACTCAGAC |
| | TCCACGATACCTGATCAAGACCAGGGGCCAGCAGGTGACACTGTCTTGCAGCCCA |
| | ATCTCCGGACACCGCTCCGTGTCTTGGTACCAGCAGACCCCTGGACAGGGACTGC |
| | AGTTCCTGTTTGAGTATTTCTCTGAGACACAGCGGAACAAGGGCAATTTCCCCGG |
| | CCGGTTTAGCGGCAGACAGTTTAGCAACTCCAGGTCTGAGATGAATGTGAGCACC |
| | CTGGAGCTGGGCGACTCCGCCCTGTACCTGTGCGCAAGCTCCCTGGCCCTGGGAC |
| | AGGGGAATCAGCAGTTTTTCGGACCCGGAACAAGGCTGACCGTGCTGGAGGATCT |
| | ACGTAACGTGACACCACCCAAAGTCTCACTGTTTGAGCCTAGCAAGGCAGAAATT |
| | GCCAACAAGCAGAAGGCCACCCTGGTGTGCCTGGCAAGAGGGTTCTTTCCAGATC |
| | ACGTGGAGCTGTCCTGGTGGGTCAACGGCAAAGAAGTGCATTCTGGGGTCTGCAC |
| | CGACCCCCAGGCTTACAAGGAGAGTAATTACTCATATTGTCTGTCAAGCCGGCTG |
| | AGAGTGTCCGCCACATTCTGGCACAACCCTAGGAATCATTTCCGCTGCCAGGTCC |
| | AGTTTCACGGCCTGAGTGAGGAAGATAAATGGCCAGAGGGGTCACCTAAGCCAGT |
| | GACACAGAACATCAGCGCAGAAGCCTGGGGACGAGCAGACTGTGGCATTACTAGC |
| | GCCTCCTATCATCAGGGCGTGCTGAGCGCCACTATCCTGTACGAGATTCTGCTGG |
| | GAAAGGCCACCCTGTATGCTGTGCTGGTCTCCGGCCTGGTGCTGATGGCCATGGT |
| | CAAGAAAAAGAACTCTGGGAGTGGAGCCACAAATTTCTCTCTGCTGAAACAGGCT |
| | GGAGATGTGGAGGAAAACCCCGGCCCTATGAAGAGCCTGCGCGTGCTGCTGGTCA |
| | TCCTGTGGCTGCAATTGAGTTGGGTGTGGAGCCAGAAGGACCAGGTGTTTCAGCC |
| | TAGCACTGTCGCATCATCAGAGGGGGCCGTCGTGGAAATCTTTTGTAATCACAGC |
| | GTGTCCAACGCCTACAATTTCTTTTGGTATCTGCACTTCCCAGGATGCGCACCTA |
| | GGCTGCTGGTGAAGGGCTCTAAGCCCAGCCAGCAGGGCCGGTACAACATGACCTA |
| | TGAGCGGTTCAGCTCCTCTCTGCTGATCCTGCAGGTGCGGGAGGCAGACGCAGCC |
| | GTGTACTATTGTGCCGTGGAGGATCTGAATCAGGCCGGCACAGCCCTGATTTTCG |
| | GCAAAGGAACAACCCTGAGCGTGAGCAGCGACATCCAGAACCCTGAGCCCGCCGT |
| | ATACCAGCTGAAGGACCCCCGATCTCAGGATAGTACTCTGTGCCTGTTCACCGAC |
| | TTTGATAGTCAGATCAATGTGCCTAAAACCATGGAATCCGGAACTTTTATTACCG |
| | ACAAGTGCGTGCTGGATATGAAAGCCATGGACAGTAAGTCAAACGGCGCCATCGC |
| | TTGGAGCAATCAGACATCCTTCACTTGCCAGGATATCTTCAAGGAGACCAACGCA |
| | ACATACCCATCCTCTGACGTGCCCTGTGATGCCACCCTGACAGAGAAGTCTTTCG |
| | AAACAGACATGAACCTGAATTTTCAGAATCTGAGCGTGATGGGCCTGAGAATCCT |
| | GCTGCTGAAGGTCGCTGGGTTTAATCTGCTGATGACACTGCGGCTGTGGTCCTCA |
| | GGATCTGGTGCTACTAATTTTTCTCTTTTGAAGCAAGCTGGAGATGTTGAAGAGA |
| | ACCCCGGTCCGATGGCTCTGCCAGTGACAGCTCTGCTGCTTCCTCTGGCTCTGTT |
| | GCTGCATGCCGCCAGACCTATTCTGTGGCACGAGATGTGGCATGAAGGCCTGGAA |
| | GAGGCCTCCAGACTGTACTTCGGCGAGAGAAACGTGAAGGGCATGTTCGAGGTGC |
| | TGGAACCCCTGCATGCCATGATGGAAAGAGGCCCTCAGACACTGAAAGAGACTAG |
| | CTTCAACCAGGCCTACGGCCGGGATCTGATGGAAGCCCAAGAGTGGTGCCGGAAG |
| | TACATGAAGTCCGGCAATGTGAAGGACCTCCTGCAGGCATGGGACCTGTACTACC |
| | ACGTGTTCCGGQGGATCTCTAAGGGCAAAGACACAATCCCTTGGCTGGGCCATCT |
| | GCTCGTTGGACTGTCTGGCGCCTTCGGCTTCATCATCCTGGTGTACCTGCTGATC |
| | AACTGTCGGAACACAGGCCCATGGCTGAAGAAAGTGCTGAAGTGCAACACCCCTG |
| | ATCCGAGCAAGTTCTTTAGCCAGCTGTCCAGCGAGCACGGCGGAGATGTTCAGAA |
| | GTGGCTGAGCAGCCCATTTCCTAGCAGCAGCTTTAGCCCTGGCGGACTGGCTCCT |
| | GAGATCAGCCCACTGGAAGTGCTGGAAAGGGACAAAGTGACCCAGCTGCTCCTGC |
| | AACAGGACAAGGTGCCAGAACCTGCCAGCCTGTCTTAAAAGCTTAATTGAGCGGC |
| | CGCGCTTTATTTGTGAAATTTGTGATGCTATTGCTTTATTTGTAACCATTATAAG |
| | CTGCAATAAACAAGTTAACAACAACAATTGCATTCATTTTATGTTTCAGGTTCAG |
| | GGGGAGATGTGGGAGGTTTTTTAAAGCTCACCGATTTTGATTCTCAAACAAATGT |
| | GTCACAAAGTAAGGATTCTGATGTGTATATCACAGACAAAACTGTGCTAGACATG |
| | AGGTCTATGGACTTCAAGAGCAACAGTGCTGTGGCCTGGAGCAACAAATCTGACT |
| | TTGCATGTGCAAACGCCTTCAACAACAGCATTATTCCAGAAGACACCTTCTTCCC |
| | CAGCCCAGGTAAGGGCAGCTTTGGTGCCTTCGCAGGCTGTTTCCTTGCTTCAGGA |
| | ATGGCCAGGTTCTGCCCAGAGCTCTGGTCAATGATGTCTAAAACTCCTCTGATTG |
| | GTGGTCTCGGCGCGGCCGCTACGTAGATAAGTAGCATGGCGGGTTAATCATTAAC |
| | TACAAGGAACCCCTAGTGATGGAGTTGGCCACTCCCTCTCTGCGCGCTCGCTCGC |
| | TCACTGAGGCCGGGCGACCAAAGGTCGCCCGACGCCCGGGCTTTGCCCGGGCGGC |
| | CTCAGTGAGCGAGCGAGCGCGCCAGCTGGCGTAATAGCGAAGAGGCCCGCACCGA |
| | TCGCCCTTCCCAACAGTTGCGCAGCCTGAATGGCGAATGGCGATTCCGTTGCAAT |
| | GGCTGGCGGTAATATTGTTCTGGATATTACCAGCAAGGCCGATAGTTTGAGTTCT |
| | TCTACTCAGGCAAGTGATGTTATTACTAATCAAAGAAGTATTGCGACAACGGTTA |
| | ATTTGCGTGATGGACAGACTCTTTTACTCGGTGGCCTCACTGATTATAAAAACAC |
| | TTCTCAGGATTCTGGCGTACCGTTCCTGTCTAAAATCCCTTTAATCGGCCTCCTG |
| | TTTAGCTCCCGCTCTGATTCTAACGAGGAAAGCACGTTATACGTGCTCGTCAAAG |
| | CAACCATAGTACGCGCCCTGTAGCGGCGCATTAAGCGCGGCGGGTGTGGTGGTTA |
| | CGCGCAGCGTGACCGCTACACTTGCCAGCGCCCTAGCGCCCGCTCCTTTCGCTTT |
| | CTTCCCTTCCTTTCTCGCCACGTTCGCCGGCTTTCCCCGTCAAGCTCTAAATCGG |
| | GGGCTCCCTTTAGGGTTCCGATTTAGTGCTTTACGGCACCTCGACCCCAAAAAAC |
| | TTGATTAGGGTGATGGTTCACGTAGTGGGCCATCGCCCTGATAGACGGTTTTTCG |
| | CCCTTTGACGTTGGAGTCCACGTTCTTTAATAGTGGACTCTTGTTCCAAACTGGA |
| | ACAACACTCAACCCTATCTCGGTCTATTCTTTTGATTTATAAGGGATTTTGCCGA |
| | TTTCGGCCTATTGGTTAAAAAATGAGCTGATTTAACAAAAATTTAACGCGAATTT |
| | TAACAAAATATTAACGTTTACAATTTAAATATTTGCTTATACAATCTTCCTGTTT |
| | TTGGGGCTTTTCTGATTATCAACCGGGGTACATATGATTGACATGCTAGTTTTAC |
| | GATTACCGTTCATCGATTCTCTTGTTTGCTCCAGACTCTCAGGCAATGACCTGAT |
| | AGCCTTTGTAGAGACCTCTCAAAAATAGCTACCCTCTCCGGCATGAATTTATCAG |
| | CTAGAACGGTTGAATATCATATTGATGGTGATTTGACTGTCTCCGGCCTTTCTCA |
| | CCCGTTTGAATCTTTACCTACACATTACTCAGGCATTGCATTTAAAATATATGAG |
| | GGTTCTAAAAATTTTTATCCTTGCGTTGAAATAAAGGCTTCTCCCGCAAAAGTAT |
| | TACAGGGTCATAATGTTTTTGGTACAACCGATTTAGCTTTATGCTCTGAGGCTTT |
| | ATTGCTTAATTTTGCTAATTCTTTGCCTTGCCTGTATGATTTATTGGATGTTGGA |
| | ATCGCCTGATGCGGTATTTTCTCCTTACGCATCTGTGCGGTATTTCACACCGCAT |
| | ATGGTGCACTCTCAGTACAATCTGCTCTGATGCCGCATAGTTAAGCCAGCCCCGA |
| | CACCCGCCAACACCCGCTGACGCGCCCTGACGGGCTTGTCTGCTCCCGGCATCCG |
| | CTTACAGACAAGCTGTGACCGTCTCCGGGAGCTGCATGTGTCAGAGGTTTTCACC |
| | GTCATCACCGAAACGCGCGAGACGAAAGGGCCTCGTGATACGCCTATTTTTATAG |
| | GTTAATGTCATGATAATAATGGTTTCTTAGACGTCAGGTGGCACTTTTCGGGGAA |
| | ATGTGCGCGGAACCCCTATTTGTTTATTTTTCTAAATACATTCAAATATGTATCC |
| | GCTCATGAGACAATAACCCTGATAAATGCTTCAATAATATTGAAAAAGGAAGAGT |
| | ATGAGTATTCAACATTTCCGTGTCGCCCTTATTCCCTTTTTTGCGGCATTTTGCC |
| | TTCCTGTTTTTGCTCACCCAGAAACGCTGGTGAAAGTAAAAGATGCTGAAGATCA |
| | GTTGGGTGCACGAGTGGGTTACATCGAACTGGATCTCAACAGCGGTAAGATCCTT |
| | GAGAGTTTTCGCCCCGAAGAACGTTTTCCAATGATGAGCACTTTTAAAGTTCTGC |
| | TATGTGGCGCGGTATTATCCCGTATTGACGCCGGGCAAGAGCAACTCGGTCGCCG |
| | CATACACTATTCTCAGAATGACTTGGTTGAGTACTCACCAGTCACAGAAAAGCAT |
| | CTTACGGATGGCATGACAGTAAGAGAATTATGCAGTGCTGCCATAACCATGAGTG |
| | ATAACACTGCGGCCAACTTACTTCTGACAACGATCGGAGGACCGAAGGAGCTAAC |
| | CGCTTTTTTGCACAACATGGGGGATCATGTAACTCGCCTTGATCGTTGGGAACCG |
| | GAGCTGAATGAAGCCATACCAAACGACGAGCGTGACACCACGATGCCTGTAGCAA |
| | TGGCAACAACGTTGCGCAAACTATTAACTGGCGAACTACTTACTCTAGCTTCCCG |
| | GCAACAATTAATAGACTGGATGGAGGCGGATAAAGTTGCAGGACCACTTCTGCGC |
| | TCGGCCCTTCCGGCTGGCTGGTTTATTGCTGATAAATCTGGAGCCGGTGAGCGTG |
| | GGTCTCGCGGTATCATTGCAGCACTGGGGCCAGATGGTAAGCCCTCCCGTATCGT |
| | AGTTATCTACACGACGGGGAGTCAGGCAACTATGGATGAACGAAATAGACAGATC |
| | GCTGAGATAGGTGCCTCACTGATTAAGCATTGGTAACTGTCAGACCAAGTTTACT |
| | CATATATACTTTAGATTGATTTAAAACTTCATTTTTAATTTAAAAGGATCTAGGT |
| | GAAGATCCTTTTTGATAATCTCATGACCAAAATCCCTTAACGTGAGTTTTCGTTC |
| | CACTGAGCGTCAGACCCC |
| Construct | GTAGAAAAGATCAAAGGATCTTCTTGAGATCCTTTTTTTCTGCGCGTAATCTGCT |
| 3323 | GCTTGCAAACAAAAAAACCACCGCTACCAGCGGTGGTTTGTTTGCCGGATCAAGA |
| SEQ ID | GCTACCAACTCTTTTTCCGAAGGTAACTGGCTTCAGCAGAGCGCAGATACCAAAT |
| NO: 1444 | ACTGTCCTTCTAGTGTAGCCGTAGTTAGGCCACCACTTCAAGAACTCTGTAGCAC |
| | CGCCTACATACCTCGCTCTGCTAATCCTGTTACCAGTGGCTGCTGCCAGTGGCGA |
| | TAAGTCGTGTCTTACCGGGTTGGACTCAAGACGATAGTTACCGGATAAGGCGCAG |
| | CGGTCGGGCTGAACGGGGGGTTCGTGCACACAGCCCAGCTTGGAGCGAACGACCT |
| | ACACCGAACTGAGATACCTACAGCGTGAGCTATGAGAAAGCGCCACGCTTCCCGA |
| | AGGGAGAAAGGCGGACAGGTATCCGGTAAGCGGCAGGGTCGGAACAGGAGAGCGC |
| | ACGAGGGAGCTTCCAGGGGGAAACGCCTGGTATCTTTATAGTCCTGTCGGGTTTC |
| | GCCACCTCTGACTTGAGCGTCGATTTTTGTGATGCTCGTCAGGGGGGCGGAGCCT |
| | ATGGAAAAACGCCAGCAACGCGGCCTTTTTACGGTTCCTGGCCTTTTGCTGGCCT |
| | TTTGCTCACATGTTCTTTCCTGCGTTATCCCCTGATTCTGTGGATAACCGTATTA |
| | CCGCCTTTGAGTGAGCTGATACCGCTCGCCGCAGCCGAACGACCGAGCGCAGCGA |
| | GTCAGTGAGCGAGGAAGCGGAAGAGCGCCCAATACGCAAACCGCCTCTCCCCGCG |
| | CGTTGGCCGATTCATTAATGCAGCTGCGCGCTCGCTCGCTCACTGAGGCCGCCCG |
| | GGCAAAGCCCGGGCGTCGGGCGACCTTTGGTCGCCCGGCCTCAGTGAGCGAGCGA |
| | GCGCGCAGAGAGGGAGTGGCCAACTCCATCACTAGGGGTTCCTTGTAGTTAATGA |
| | TTAACCCGCCATGCTACTTATCTACGTACCCTATTAAATAAAAGAATAAGCAGTA |
| | TTATTAAGTAGCCCTGCATTTCAGGTTTCCTTGAGTGGCAGGCCAGGCCTGGCCG |
| | TGAACGTTCACTGAAATCATGGCCTCTTGGCCAAGATTGATAGCTTGTGCCTGTC |
| | CCTGAGTCCCAGTCCATCACGAGCAGCTGGTTTCTAAGATGCTATTTCCCGTATA |
| | AAGCATGAGACCGTGACTTGCCAGCCCCACAGAGCCCCGCCCTTGTCCATCACTG |
| | GCATCTGGACTCCAGCCTGGGTTGGGGCAAAGAGGGAAATGAGATCATGTCCTAC |
| | GCGTAGGAACAGAGAAACAGGAGAATATGGGCCAAACAGGATATCTGTGGTAAGC |
| | AGTTCCTGCCCCGGCTCAGGGCCAAGAACAGTTGGAACAGCAGAATATGGGCCAA |
| | ACAGGATATCTGTGGTAAGCAGTTCCTGCCCCGGCTCAGGGCCAAGAACAGATGG |
| | TCCCCAGATGCGGTCCCGCCCTCAGCAGTTTCTAGAGAACCATCAGATGTTTCCA |
| | GGGTGCCCCAAGGACCTGAAATGACCCTGTGCCTTATTTGAACTAACCAATCAGT |
| | TCGCTTCTCGCTTCTGTTCGCGCGCTTCTGCTCCCCGAGCTCTATATAAGCAGAG |
| | CTCGTTTAGTGAACCGTCAGATCGCCTGGAGACGCCATCCACGCTGTTTTGACTT |
| | CCATAGAAGGATCATGGCTCTGCCAGTGACAGCTCTGCTGCTTCCTCTGGCTCTG |
| | TTGCTGCATGCCGCCAGACCTATTCTGTGGCACGAGATGTGGCATGAAGGCCTGG |
| | AAGAGGCCTCCAGACTGTACTTCGGCGAGAGAAACGTGAAGGGCATGTTCGAGGT |
| | GCTGGAACCCCTGCATGCCATGATGGAAAGAGGCCCTCAGACACTGAAAGAGACA |
| | AGCTTCAACCAGGCCTACGGCCGGGATCTGATGGAAGCCCAAGAGTGGTGCCGGA |
| | AGTACATGAAGTCCGGCAATGTGAAGGACCTCCTGCAGGCATGGGACCTGTACTA |
| | CCACGTGTTCCGGQGGATCTCTAAGGGCAAAGACACAATCCCTTGGCTGGGCCAT |
| | CTGCTCGTTGGACTGTCTGGCGCCTTCGGCTTCATCATCCTGGTGTACCTGCTGA |
| | TCAACTGTCGGAACACAGGCCCATGGCTGAAGAAAGTGCTGAAGTGCAACACCCC |
| | TGATCCGAGCAAGTTCTTTAGCCAGCTGTCCAGCGAGCACGGCGGAGATGTTCAG |
| | AAGTGGCTGAGCAGCCCATTTCCTAGCAGCAGCTTTAGCCCTGGCGGACTGGCTC |
| | CTGAGATCAGCCCACTGGAAGTGCTGGAAAGGGACAAAGTGACCCAGCTGCTCCT |
| | GCAACAGGACAAGGTGCCAGAACCTGCCAGCCTGTCTCTGAACACCGATGCCTAT |
| | CTGTCCCTGCAAGAGCTGCAAGGATCCGGCGCCACCAACTTTAGTCTGCTCAAGC |
| | AAGCCGGGGACGTCGAGGAAAATCCTGGGCCAATGGGATCAAGACTGCTGTGCTG |
| | GGTGCTGCTGTGCCTGCTGGGAGCCGGACCTGTGAAAGCCGGGGTGACTCAGACT |
| | CCACGATACCTGATCAAGACCAGGGGCCAGCAGGTGACACTGTCTTGCAGCCCAA |
| | TCTCCGGACACCGCTCCGTGTCTTGGTACCAGCAGACCCCTGGACAGGGACTGCA |
| | GTTCCTGTTTGAGTATTTCTCTGAGACACAGCGGAACAAGGGCAATTTCCCCGGC |
| | CGGTTTAGCGGCAGACAGTTTAGCAACTCCAGGTCTGAGATGAATGTGAGCACCC |
| | TGGAGCTGGGCGACTCCGCCCTGTACCTGTGCGCAAGCTCCCTGGCCCTGGGACA |
| | GGGGAATCAGCAGTTTTTCGGACCCGGAACAAGGCTGACCGTGCTGGAGGATCTA |
| | CGTAACGTGACACCACCCAAAGTCTCACTGTTTGAGCCTAGCAAGGCAGAAATTG |
| | CCAACAAGCAGAAGGCCACCCTGGTGTGCCTGGCAAGAGGGTTCTTTCCAGATCA |
| | CGTGGAGCTGTCCTGGTGGGTCAACGGCAAAGAAGTGCATTCTGGGGTCTGCACC |
| | GACCCCCAGGCTTACAAGGAGAGTAATTACTCATATTGTCTGTCAAGCCGGCTGA |
| | GAGTGTCCGCCACATTCTGGCACAACCCTAGGAATCATTTCCGCTGCCAGGTCCA |
| | GTTTCACGGCCTGAGTGAGGAAGATAAATGGCCAGAGGGGTCACCTAAGCCAGTG |
| | ACACAGAACATCAGCGCAGAAGCCTGGGGACGAGCAGACTGTGGCATTACTAGCG |
| | CCTCCTATCATCAGGGCGTGCTGAGCGCCACTATCCTGTACGAGATTCTGCTGGG |
| | AAAGGCCACCCTGTATGCTGTGCTGGTCTCCGGCCTGGTGCTGATGGCCATGGTC |
| | AAGAAAAAGAACTCTGGGAGTGGAGCCACAAATTTCTCTCTGCTGAAACAGGCTG |
| | GAGATGTGGAGGAAAACCCCGGCCCTATGAAGAGCCTGCGCGTGCTGCTGGTCAT |
| | CCTGTGGCTGCAATTGAGTTGGGTGTGGAGCCAGAAGGACCAGGTGTTTCAGCCT |
| | AGCACTGTCGCATCATCAGAGGGGGCCGTCGTGGAAATCTTTTGTAATCACAGCG |
| | TGTCCAACGCCTACAATTTCTTTTGGTATCTGCACTTCCCAGGATGCGCACCTAG |
| | GCTGCTGGTGAAGGGCTCTAAGCCCAGCCAGCAGGGCCGGTACAACATGACCTAT |
| | GAGCGGTTCAGCTCCTCTCTGCTGATCCTGCAGGTGCGGGAGGCAGACGCAGCCG |
| | TGTACTATTGTGCCGTGGAGGATCTGAATCAGGCCGGCACAGCCCTGATTTTCGG |
| | CAAAGGAACAACCCTGAGCGTGAGCAGCATCCAGAACCCTGACCCAGCCGTATAC |
| | CAGCTGAGAGACTCTAAATCCAGTGACAAGTCTGTCTGCCTATTCACCGATTTTG |
| | ATTCTCAAACAAATGTGTCACAAAGTAAGGATTCTGATGTGTATATCACAGACAA |
| | AACTGTGCTAGACATGAGGTCTATGGACTTCAAGAGCAACAGTGCTGTGGCCTGG |
| | AGCAACAAATCTGACTTTGCATGTGCAAACGCCTTCAACAACAGCATTATTCCAG |
| | AAGACACCTTCTTCCCCAGCCCAGGTAAGGGCAGCTTTGGTGCCTTCGCAGGCTG |
| | TTTCCTTGCTTCAGGAATGGATCCTACGTAGATAAGTAGCATGGGGGGTTAATCA |
| | TTAACTACAAGGAACCCCTAGTGATGGAGTTGGCCACTCCCTCTCTGCGCGCTCG |
| | CTCGCTCACTGAGGCCGGGCGACCAAAGGTCGCCCGACGCCCGGGCTTTGCCCGG |
| | GCGGCCTCAGTGAGCGAGCGAGCGCGCCAGCTGGCGTAATAGCGAAGAGGCCCGC |
| | ACCGATCGCCCTTCCCAACAGTTGCGCAGCCTGAATGGCGAATGGCGATTCCGTT |
| | GCAATGGCTGGCGGTAATATTGTTCTGGATATTACCAGCAAGGCCGATAGTTTGA |
| | GTTCTTCTACTCAGGCAAGTGATGTTATTACTAATCAAAGAAGTATTGCGACAAC |
| | GGTTAATTTGCGTGATGGACAGACTCTTTTACTCGGTGGCCTCACTGATTATAAA |
| | AACACTTCTCAGGATTCTGGCGTACCGTTCCTGTCTAAAATCCCTTTAATCGGCC |
| | TCCTGTTTAGCTCCCGCTCTGATTCTAACGAGGAAAGCACGTTATACGTGCTCGT |
| | CAAAGCAACCATAGTACGCGCCCTGTAGCGGCGCATTAAGCGCGGCGGGTGTGGT |
| | GGTTACGCGCAGCGTGACCGCTACACTTGCCAGCGCCCTAGCGCCCGCTCCTTTC |
| | GCTTTCTTCCCTTCCTTTCTCGCCACGTTCGCCGGCTTTCCCCGTCAAGCTCTAA |
| | ATCGGGGGCTCCCTTTAGGGTTCCGATTTAGTGCTTTACGGCACCTCGACCCCAA |
| | AAAACTTGATTAGGGTGATGGTTCACGTAGTGGGCCATCGCCCTGATAGACGGTT |
| | TTTCGCCCTTTGACGTTGGAGTCCACGTTCTTTAATAGTGGACTCTTGTTCCAAA |
| | CTGGAACAACACTCAACCCTATCTCGGTCTATTCTTTTGATTTATAAGGGATTTT |
| | GCCGATTTCGGCCTATTGGTTAAAAAATGAGCTGATTTAACAAAAATTTAACGCG |
| | AATTTTAACAAAATATTAACGTTTACAATTTAAATATTTGCTTATACAATCTTCC |
| | TGTTTTTGGGGCTTTTCTGATTATCAACCGGGGTACATATGATTGACATGCTAGT |
| | TTTACGATTACCGTTCATCGATTCTCTTGTTTGCTCCAGACTCTCAGGCAATGAC |
| | CTGATAGCCTTTGTAGAGACCTCTCAAAAATAGCTACCCTCTCCGGCATGAATTT |
| | ATCAGCTAGAACGGTTGAATATCATATTGATGGTGATTTGACTGTCTCCGGCCTT |
| | TCTCACCCGTTTGAATCTTTACCTACACATTACTCAGGCATTGCATTTAAAATAT |
| | ATGAGGGTTCTAAAAATTTTTATCCTTGCGTTGAAATAAAGGCTTCTCCCGCAAA |
| | AGTATTACAGGGTCATAATGTTTTTGGTACAACCGATTTAGCTTTATGCTCTGAG |
| | GCTTTATTGCTTAATTTTGCTAATTCTTTGCCTTGCCTGTATGATTTATTGGATG |
| | TTGGAATCGCCTGATGCGGTATTTTCTCCTTACGCATCTGTGCGGTATTTCACAC |
| | CGCATATGGTGCACTCTCAGTACAATCTGCTCTGATGCCGCATAGTTAAGCCAGC |
| | CCCGACACCCGCCAACACCCGCTGACGCGCCCTGACGGGCTTGTCTGCTCCCGGC |
| | ATCCGCTTACAGACAAGCTGTGACCGTCTCCGGGAGCTGCATGTGTCAGAGGTTT |
| | TCACCGTCATCACCGAAACGCGCGAGACGAAAGGGCCTCGTGATACGCCTATTTT |
| | TATAGGTTAATGTCATGATAATAATGGTTTCTTAGACGTCAGGTGGCACTTTTCG |
| | GGGAAATGTGCGCGGAACCCCTATTTGTTTATTTTTCTAAATACATTCAAATATG |
| | TATCCGCTCATGAGACAATAACCCTGATAAATGCTTCAATAATATTGAAAAAGGA |
| | AGAGTATGAGTATTCAACATTTCCGTGTCGCCCTTATTCCCTTTTTTGCGGCATT |
| | TTGCCTTCCTGTTTTTGCTCACCCAGAAACGCTGGTGAAAGTAAAAGATGCTGAA |
| | GATCAGTTGGGTGCACGAGTGGGTTACATCGAACTGGATCTCAACAGCGGTAAGA |
| | TCCTTGAGAGTTTTCGCCCCGAAGAACGTTTTCCAATGATGAGCACTTTTAAAGT |
| | TCTGCTATGTGGCGCGGTATTATCCCGTATTGACGCCGGGCAAGAGCAACTCGGT |
| | CGCCGCATACACTATTCTCAGAATGACTTGGTTGAGTACTCACCAGTCACAGAAA |
| | AGCATCTTACGGATGGCATGACAGTAAGAGAATTATGCAGTGCTGCCATAACCAT |
| | GAGTGATAACACTGCGGCCAACTTACTTCTGACAACGATCGGAGGACCGAAGGAG |
| | CTAACCGCTTTTTTGCACAACATGGGGGATCATGTAACTCGCCTTGATCGTTGGG |
| | AACCGGAGCTGAATGAAGCCATACCAAACGACGAGCGTGACACCACGATGCCTGT |
| | AGCAATGGCAACAACGTTGCGCAAACTATTAACTGGCGAACTACTTACTCTAGCT |
| | TCCCGGCAACAATTAATAGACTGGATGGAGGCGGATAAAGTTGCAGGACCACTTC |
| | TGCGCTCGGCCCTTCCGGCTGGCTGGTTTATTGCTGATAAATCTGGAGCCGGTGA |
| | GCGTGGGTCTCGCGGTATCATTGCAGCACTGGGGCCAGATGGTAAGCCCTCCCGT |
| | ATCGTAGTTATCTACACGACGGGGAGTCAGGCAACTATGGATGAACGAAATAGAC |
| | AGATCGCTGAGATAGGTGCCTCACTGATTAAGCATTGGTAACTGTCAGACCAAGT |
| | TTACTCATATATACTTTAGATTGATTTAAAACTTCATTTTTAATTTAAAAGGATC |
| | TAGGTGAAGATCCTTTTTGATAATCTCATGACCAAAATCCCTTAACGTGAGTTTT |
| | CGTTCCACTGAGCGTCAGACCCC |
| Construct | AGCTTAATGTAGTCTTATGCAATACTCTTGTAGTCTTGCAACATGGTAACGATGA |
| 3312 | GTTAGCAACATGCCTTACAAGGAGAGAAAAAGCACCGTGCATGCCGATTGGTGGA |
| SEQ ID | AGTAAGGTGGTACGATCGTGCCTTATTAGGAAGGCAACAGACGGGTCTGACATGG |
| NO: 1445 | ATTGGACGAACCACTGAATTGCCGCATTGCAGAGATATTGTATTTAAGTGCCTAG |
| | CTCGATACAATAAACGGGTCTCTCTGGTTAGACCAGATCTGAGCCTGGGAGCTCT |
| | CTGGCTAACTAGGGAACCCACTGCTTAAGCCTCAATAAAGCTTGCCTTGAGTGCT |
| | TCAAGTAGTGTGTGCCCGTCTGTTGTGTGACTCTGGTAACTAGAGATCCCTCAGA |
| | CCCTTTTAGTCAGTGTGGAAAATCTCTAGCAGTGGCGCCCGAACAGGGACTTGAA |
| | AGCGAAAGGGAAACCAGAGGAGCTCTCTCGACGCAGGACTCGGCTTGCTGAAGCG |
| | CGCACGGCAAGAGGCGAGGGGCGGCGACTGGTGAGTACGCCAAAAATTTTGACTA |
| | GCGGAGGCTAGAAGGAGAGAGATGGGTGCGAGAGCGTCAGTATTAAGCGGGGGAG |
| | AATTAGATCGCGATGGGAAAAAATTCGGTTAAGGCCAGGGGGAAAGAAAAAATAT |
| | AAATTAAAACATATAGTATGGGCAAGCAGGGAGCTAGAACGATTCGCAGTTAATC |
| | CTGGCCTGTTAGAAACATCAGAAGGCTGTAGACAAATACTGGGACAGCTACAACC |
| | ATCCCTTCAGACAGGATCAGAAGAACTTAGATCATTATATAATACAGTAGCAACC |
| | CTCTATTGTGTGCATCAAAGGATAGAGATAAAAGACACCAAGGAAGCTTTAGACA |
| | AGATAGAGGAAGAGCAAAACAAAAGTAAGACCACCGCACAGCAAGCGGCCGCTGA |
| | TCTTCAGACCTGGAGGAGGAGATATGAGGGACAATTGGAGAAGTGAATTATATAA |
| | ATATAAAGTAGTAAAAATTGAACCATTAGGAGTAGCACCCACCAAGGCAAAGAGA |
| | AGAGTGGTGCAGAGAGAAAAAAGAGCAGTGGGAATAGGAGCTTTGTTCCTTGGGT |
| | TCTTGGGAGCAGCAGGAAGCACTATGGGCGCAGCCTCAATGACGCTGACGGTACA |
| | GGCCAGACAATTATTGTCTGGTATAGTGCAGCAGCAGAACAATTTGCTGAGGGCT |
| | ATTGAGGCGCAACAGCATCTGTTGCAACTCACAGTCTGGGGCATCAAGCAGCTCC |
| | AGGCAAGAATCCTGGCTGTGGAAAGATACCTAAAGGATCAACAGCTCCTGGGGAT |
| | TTGGGGTTGCTCTGGAAAACTCATTTGCACCACTGCTGTGCCTTGGAATGCTAGT |
| | TGGAGTAATAAATCTCTGGAACAGATTTGGAATCACACGACCTGGATGGAGTGGG |
| | ACAGAGAAATTAACAATTACACAAGCTTAATACACTCCTTAATTGAAGAATCGCA |
| | AAACCAGCAAGAAAAGAATGAACAAGAATTATTGGAATTAGATAAATGGGCAAGT |
| | TTGTGGAATTGGTTTAACATAACAAATTGGCTGTGGTATATAAAATTATTCATAA |
| | TGATAGTAGGAGGCTTGGTAGGTTTAAGAATAGTTTTTGCTGTACTTTCTATAGT |
| | GAATAGAGTTAGGCAGGGATATTCACCATTATCGTTTCAGACCCACCTCCCAACC |
| | CCGAGGGGACCCGACAGGCCCGAAGGAATAGAAGAAGAAGGTGGAGAGAGAGACA |
| | GAGACAGATCCATTCGATTAGTGAACGGATCTCGACGGTATCGGTTAACTTTTAA |
| | AAGAAAAGGGGGGATTGGGGGGTACAGTGCAGGGGAAAGAATAGTAGACATAATA |
| | GCAACAGACATACAAACTAAAGAATTACAAAAACAAATTACAAAAATTCAAAATT |
| | TTATCGATCACGAGACTAGCCTCGAGAAGCTTGATATCGAATTCCCACGGGGTTG |
| | GACGCGTAGAGGCTCCGGTGCCCGTCAGTGGGCAGAGCGCACATCGCCCACAGTC |
| | CCCGAGAAGTTGGGGGGAGGGGTCGGCAATTGAACCGGTGCCTAGAGAAGGTGGC |
| | GCGGGGTAAACTGGGAAAGTGATGTCGTGTACTGGCTCCGCCTTTTTCCCGAGGG |
| | TGGGGGAGAACCGTATATAAGTGCAGTAGTCGCCGTGAACGTTCTTTTTCGCAAC |
| | GGGTTTGCCGCCAGAACACAGCTAGCACCGGTGCCGCCACCATGCCTCTGGGCCT |
| | GCTGTGGCTGGGCCTGGCCCTGCTGGGCGCCCTGCACGCCCAGGCCGGCGTGCAG |
| | GTGGAGACAATCTCCCCAGGCGACGGACGCACATTCCCTAAGCGGGGCCAGACCT |
| | GCGTGGTGCACTATACAGGCATGCTGGAGGATGGCAAGAAGTTTGACAGCTCCCG |
| | GGATAGAAACAAGCCATTCAAGTTTATGCTGGGCAAGCAGGAAGTGATCAGAGGC |
| | TGGGAGGAGGGCGTGGCCCAGATGTCTGTGGGCCAGAGGGCCAAGCTGACCATCA |
| | GCCCAGACTACGCCTATGGAGCAACAGGCCACCCAGGAATCATCCCACCTCACGC |
| | CACCCTGGTGTTCGATGTGGAGCTGCTGAAGCTGGGCGAGGGATCCAACACATCA |
| | AAAGAGAACCCCTTTCTGTTCGCATTGGAGGCCGTAGTCATATCTGTTGGATCCA |
| | TGGGACTTATTATCTCCCTGTTGTGTGTGTACTTCTGGCTGGAACGGACTATGCC |
| | CAGGATCCCCACGCTCAAGAATCTGGAAGATCTCGTCACAGAATACCATGGTAAT |
| | TTCAGCGCCTGGAGCGGAGTCTCTAAGGGTCTGGCCGAATCCCTCCAACCCGATT |
| | ATTCTGAACGGTTGTGCCTCGTATCCGAAATACCACCAAAAGGCGGGGCTCTGGG |
| | TGAGGGCCCAGGGGCGAGTCCGTGCAATCAACACAGCCCGTATTGGGCCCCTCCT |
| | TGTTATACGTTGAAGCCCGAAACTGGAAGCGGAGCTACTAACTTCAGCCTGCTGA |
| | AGCAGGCTGGAGACGTGGAGGAGAACCCTGGACCTATGGCACTGCCCGTGACCGC |
| | CCTGCTGCTGCCTCTGGCCCTGCTGCTGCACGCAGCCCGGCCTATCCTGTGGCAC |
| | GAGATGTGGCACGAGGGCCTGGAGGAGGCCAGCAGGCTGTATTTTGGCGAGCGCA |
| | ACGTGAAGGGCATGTTCGAGGTGCTGGAGCCTCTGCACGCCATGATGGAGAGAGG |
| | CCCACAGACCCTGAAGGAGACATCCTTTAACCAGGCCTATGGACGGGACCTGATG |
| | GAGGCACAGGAGTGGTGCAGAAAGTACATGAAGTCTGGCAATGTGAAGGACCTGC |
| | TGCAGGCCTGGGATCTGTACTATCACGTGTTTCGGAGAATCTCCAAGGGCAAAGA |
| | CACGATTCCGTGGCTTGGGCATCTGCTCGTTGGGCTGAGTGGTGCGTTTGGTTTC |
| | ATCATCTTGGTCTATCTCTTGATCAATTGCAGAAATACAGGCCCTTGGCTGAAAA |
| | AAGTGCTCAAGTGTAATACCCCCGACCCAAGCAAGTTCTTCTCCCAGCTTTCTTC |
| | AGAGCATGGAGGCGATGTGCAGAAATGGCTCTCTTCACCTTTTCCCTCCTCAAGC |
| | TTCTCCCCGGGAGGGCTGGCGCCCGAGATTTCACCTCTTGAGGTACTTGAACGAG |
| | ACAAGGTTACCCAACTTCTCCTTCAACAGGATAAGGTACCCGAACCTGCGAGCCT |
| | TAGCTCCAACCACTCTCTTACGAGCTGCTTCACCAATCAGGGATACTTCTTTTTC |
| | CACCTTCCCGATGCGCTGGAAATCGAAGCTTGTCAAGTTTACTTTACCTATGATC |
| | CATATAGCGAGGAAGATCCCGACGAAGGAGTCGCCGGTGCGCCCACGGGTTCCTC |
| | ACCCCAACCTCTCCAGCCTCTCTCAGGAGAAGATGATGCTTATTGCACTTTTCCC |
| | AGTAGAGACGATCTCCTCCTCTTTTCTCCATCTCTTTTGGGGGGACCTTCCCCCC |
| | CTTCTACGGCACCTGGCGGGTCTGGTGCTGGCGAGGAGCGGATGCCGCCGTCCCT |
| | CCAGGAGCGAGTACCACGAGATTGGGATCCCCAGCCACTTGGACCCCCCACCCCC |
| | GGCGTACCTGACCTTGTCGATTTTCAACCTCCCCCTGAATTGGTGCTGCGAGAGG |
| | CTGGGGAGGAAGTTCCGGACGCTGGGCCGAGGGAGGGCGTGTCCTTTCCATGGAG |
| | TAGGCCTCCAGGTCAAGGCGAGTTTAGGGCTCTCAACGCGCGGCTGCCGTTGAAT |
| | ACAGACGCTTATCTCTCACTGCAGGAACTGCAAGGTCAGGACCCAACACATCTTG |
| | TAGGATCTGGTGCTACTAATTTTTCTCTTTTGAAGCAAGCTGGAGATGTTGAAGA |
| | GAACCCCGGTCCGGTGAGCAAGGGCGAGGAGGATAACATGGCCATCATCAAGGAG |
| | TTCATGCGCTTCAAGGTGCACATGGAGGGCTCCGTGAACGGCCACGAGTTCGAGA |
| | TCGAGGGCGAGGGCGAGGGCCGCCCCTACGAGGGCACCCAGACCGCCAAGCTGAA |
| | GGTGACCAAGGGTGGCCCCCTGCCCTTCGCCTGGGACATCCTGTCCCCTCAGTTC |
| | ATGTACGGCTCCAAGGCCTACGTGAAGCACCCCGCCGACATCCCCGACTACTTGA |
| | AGCTGTCCTTCCCCGAGGGCTTCAAGTGGGAGCGCGTGATGAACTTCGAGGACGG |
| | CGGCGTGGTGACCGTGACCCAGGACTCCTCTCTGCAGGACGGCGAGTTCATCTAC |
| | AAGGTGAAGCTGCGCGGCACCAACTTCCCCTCCGACGGCCCCGTAATGCAGAAGA |
| | AGACCATGGGCTGGGAGGCCTCCTCCGAGCGGATGTACCCCGAGGACGGCGCCCT |
| | GAAGGGCGAGATCAAGCAGAGGCTGAAGCTGAAGGACGGCGGCCACTACGACGCT |
| | GAGGTCAAGACCACCTACAAGGCCAAGAAGCCCGTGCAGCTGCCCGGCGCCTACA |
| | ACGTCAACATCAAGTTGGACATCACCTCCCACAACGAGGACTACACCATCGTGGA |
| | ACAGTACGAACGCGCCGAGGGCCGCCACTCCACCGGCGGCATGGACGAGCTGTAC |
| | AAGTGAACTAGTGTCGACAATCAACCTCTGGATTACAAAATTTGTGAAAGATTGA |
| | CTGGTATTCTTAACTATGTTGCTCCTTTTACGCTATGTGGATACGCTGCTTTAAT |
| | GCCTTTGTATCATGCTATTGCTTCCCGTATGGCTTTCATTTTCTCCTCCTTGTAT |
| | AAATCCTGGTTGCTGTCTCTTTATGAGGAGTTGTGGCCCGTTGTCAGGCAACGTG |
| | GCGTGGTGTGCACTGTGTTTGCTGACGCAACCCCCACTGGTTGGGGCATTGCCAC |
| | CACCTGTCAGCTCCTTTCCGGGACTTTCGCTTTCCCCCTCCCTATTGCCACGGCG |
| | GAACTCATCGCCGCCTGCCTTGCCCGCTGCTGGACAGGGGCTCGGCTGTTGGGCA |
| | CTGACAATTCCGTGGTGTTGTCGGGGAAGCTGACGTCCTTTCCATGGCTGCTCGC |
| | CTGTGTTGCCACCTGGATTCTGCGCGGGACGTCCTTCTGCTACGTCCCTTCGGCC |
| | CTCAATCCAGCGGACCTTCCTTCCCGCGGCCTGCTGCCGGCTCTGCGGCCTCTTC |
| | CGCGTCTTCGCCTTCGCCCTCAGACGAGTCGGATCTCCCTTTGGGCCGCCTCCCC |
| | GCCTGGAATTCGAGCTCGGTACCTTTAAGACCAATGACTTACAAGGCAGCTGTAG |
| | ATCTTAGCCACTTTTTAAAAGAAAAGGGGGGACTGGAAGGGCTAATTCACTCCCA |
| | ACGAAGACAAGATCTGCTTTTTGCTTGTACTGGGTCTCTCTGGTTAGACCAGATC |
| | TGAGCCTGGGAGCTCTCTGGCTAACTAGGGAACCCACTGCTTAAGCCTCAATAAA |
| | GCTTGCCTTGAGTGCTTCAAGTAGTGTGTGCCCGTCTGTTGTGTGACTCTGGTAA |
| | CTAGAGATCCCTCAGACCCTTTTAGTCAGTGTGGAAAATCTCTAGCAGTAGTAGT |
| | TCATGTCATCTTATTATTCAGTATTTATAACTTGCAAAGAAATGAATATCAGAGA |
| | GTGAGAGGAACTTGTTTATTGCAGCTTATAATGGTTACAAATAAAGCAATAGCAT |
| | CACAAATTTCACAAATAAAGCATTTTTTTCACTGCATTCTAGTTGTGGTTTGTCC |
| | AAACTCATCAATGTATCTTATCATGTCTGGCTCTAGCTATCCCGCCCCTAACTCC |
| | GCCCAGTTCCGCCCATTCTCCGCCCCATGGCTGACTAATTTTTTTTATTTATGCA |
| | GAGGCCGAGGCCGCCTCGGCCTCTGAGCTATTCCAGAAGTAGTGAGGAGGCTTTT |
| | TTGGAGGCCTAGGCTTTTGCGTCGAGACGTACCCAATTCGCCCTATAGTGAGTCG |
| | TATTACGCGCGCTCACTGGCCGTCGTTTTACAACGTCGTGACTGGGAAAACCCTG |
| | GCGTTACCCAACTTAATCGCCTTGCAGCACATCCCCCTTTCGCCAGCTGGCGTAA |
| | TAGCGAAGAGGCCCGCACCGATCGCCCTTCCCAACAGTTGCGCAGCCTGAATGGC |
| | GAATGGCGCGACGCGCCCTGTAGCGGCGCATTAAGCGCGGCGGGTGTGGTGGTTA |
| | CGCGCAGCGTGACCGCTACACTTGCCAGCGCCCTAGCGCCCGCTCCTTTCGCTTT |
| | CTTCCCTTCCTTTCTCGCCACGTTCGCCGGCTTTCCCCGTCAAGCTCTAAATCGG |
| | GGGCTCCCTTTAGGGTTCCGATTTAGTGCTTTACGGCACCTCGACCCCAAAAAAC |
| | TTGATTAGGGTGATGGTTCACGTAGTGGGCCATCGCCCTGATAGACGGTTTTTCG |
| | CCCTTTGACGTTGGAGTCCACGTTCTTTAATAGTGGACTCTTGTTCCAAACTGGA |
| | ACAACACTCAACCCTATCTCGGTCTATTCTTTTGATTTATAAGGGATTTTGCCGA |
| | TTTCGGCCTATTGGTTAAAAAATGAGCTGATTTAACAAAAATTTAACGCGAATTT |
| | TAACAAAATATTAACGTTTACAATTTCCCAGGTGGCACTTTTCGGGGAAATGTGC |
| | GCGGAACCCCTATTTGTTTATTTTTCTAAATACATTCAAATATGTATCCGCTCAT |
| | GAGACAATAACCCTGATAAATGCTTCAATAATATTGAAAAAGGAAGAGTATGAGT |
| | ATTCAACATTTCCGTGTCGCCCTTATTCCCTTTTTTGCGGCATTTTGCCTTCCTG |
| | TTTTTGCTCACCCAGAAACGCTGGTGAAAGTAAAAGATGCTGAAGATCAGTTGGG |
| | TGCACGAGTGGGTTACATCGAACTGGATCTCAACAGCGGTAAGATCCTTGAGAGT |
| | TTTCGCCCCGAAGAACGTTTTCCAATGATGAGCACTTTTAAAGTTCTGCTATGTG |
| | GCGCGGTATTATCCCGTATTGACGCCGGGCAAGAGCAACTCGGTCGCCGCATACA |
| | CTATTCTCAGAATGACTTGGTTGAGTACTCACCAGTCACAGAAAAGCATCTTACG |
| | GATGGCATGACAGTAAGAGAATTATGCAGTGCTGCCATAACCATGAGTGATAACA |
| | CTGCGGCCAACTTACTTCTGACAACGATCGGAGGACCGAAGGAGCTAACCGCTTT |
| | TTTGCACAACATGGGGGATCATGTAACTCGCCTTGATCGTTGGGAACCGGAGCTG |
| | AATGAAGCCATACCAAACGACGAGCGTGACACCACGATGCCTGTAGCAATGGCAA |
| | CAACGTTGCGCAAACTATTAACTGGCGAACTACTTACTCTAGCTTCCCGGCAACA |
| | ATTAATAGACTGGATGGAGGCGGATAAAGTTGCAGGACCACTTCTGCGCTCGGCC |
| | CTTCCGGCTGGCTGGTTTATTGCTGATAAATCTGGAGCCGGTGAGCGTGGGTCTC |
| | GCGGTATCATTGCAGCACTGGGGCCAGATGGTAAGCCCTCCCGTATCGTAGTTAT |
| | CTACACGACGGGGAGTCAGGCAACTATGGATGAACGAAATAGACAGATCGCTGAG |
| | ATAGGTGCCTCACTGATTAAGCATTGGTAACTGTCAGACCAAGTTTACTCATATA |
| | TACTTTAGATTGATTTAAAACTTCATTTTTAATTTAAAAGGATCTAGGTGAAGAT |
| | CCTTTTTGATAATCTCATGACCAAAATCCCTTAACGTGAGTTTTCGTTCCACTGA |
| | GCGTCAGACCCCGTAGAAAAGATCAAAGGATCTTCTTGAGATCCTTTTTTTCTGC |
| | GCGTAATCTGCTGCTTGCAAACAAAAAAACCACCGCTACCAGCGGTGGTTTGTTT |
| | GCCGGATCAAGAGCTACCAACTCTTTTTCCGAAGGTAACTGGCTTCAGCAGAGCG |
| | CAGATACCAAATACTGTCCTTCTAGTGTAGCCGTAGTTAGGCCACCACTTCAAGA |
| | ACTCTGTAGCACCGCCTACATACCTCGCTCTGCTAATCCTGTTACCAGTGGCTGC |
| | TGCCAGTGGCGATAAGTCGTGTCTTACCGGGTTGGACTCAAGACGATAGTTACCG |
| | GATAAGGCGCAGCGGTCGGGCTGAACGGGGGGTTCGTGCACACAGCCCAGCTTGG |
| | AGCGAACGACCTACACCGAACTGAGATACCTACAGCGTGAGCTATGAGAAAGCGC |
| | CACGCTTCCCGAAGGGAGAAAGGCGGACAGGTATCCGGTAAGCGGCAGGGTCGGA |
| | ACAGGAGAGCGCACGAGGGAGCTTCCAGGGGGAAACGCCTGGTATCTTTATAGTC |
| | CTGTCGGGTTTCGCCACCTCTGACTTGAGCGTCGATTTTTGTGATGCTCGTCAGG |
| | GGGGCGGAGCCTATGGAAAAACGCCAGCAACGCGGCCTTTTTACGGTTCCTGGCC |
| | TTTTGCTGGCCTTTTGCTCACATGTTCTTTCCTGCGTTATCCCCTGATTCTGTGG |
| | ATAACCGTATTACCGCCTTTGAGTGAGCTGATACCGCTCGCCGCAGCCGAACGAC |
| | CGAGCGCAGCGAGTCAGTGAGCGAGGAAGCGGAAGAGCGCCCAATACGCAAACCG |
| | CCTCTCCCCGCGCGTTGGCCGATTCATTAATGCAGCTGGCACGACAGGTTTCCCG |
| | ACTGGAAAGCGGGCAGTGAGCGCAACGCAATTAATGTGAGTTAGCTCACTCATTA |
| | GGCACCCCAGGCTTTACACTTTATGCTTCCGGCTCGTATGTTGTGTGGAATTGTG |
| | AGCGGATAACAATTTCACACAGGAAACAGCTATGACCATGATTACGCCAAGCGCG |
| | CAATTAACCCTCACTAAAGGGAACAAAAGCTGGAGCTGCA |
| Construct | GTAGAAAAGATCAAAGGATCTTCTTGAGATCCTTTTTTTCTGCGCGTAATCTGCT |
| 3354 | GCTTGCAAACAAAAAAACCACCGCTACCAGCGGTGGTTTGTTTGCCGGATCAAGA |
| SEQ ID | GCTACCAACTCTTTTTCCGAAGGTAACTGGCTTCAGCAGAGCGCAGATACCAAAT |
| NO: 1446 | ACTGTCCTTCTAGTGTAGCCGTAGTTAGGCCACCACTTCAAGAACTCTGTAGCAC |
| | CGCCTACATACCTCGCTCTGCTAATCCTGTTACCAGTGGCTGCTGCCAGTGGCGA |
| | TAAGTCGTGTCTTACCGGGTTGGACTCAAGACGATAGTTACCGGATAAGGCGCAG |
| | CGGTCGGGCTGAACGGGGGGTTCGTGCACACAGCCCAGCTTGGAGCGAACGACCT |
| | ACACCGAACTGAGATACCTACAGCGTGAGCTATGAGAAAGCGCCACGCTTCCCGA |
| | AGGGAGAAAGGCGGACAGGTATCCGGTAAGCGGCAGGGTCGGAACAGGAGAGCGC |
| | ACGAGGGAGCTTCCAGGGGGAAACGCCTGGTATCTTTATAGTCCTGTCGGGTTTC |
| | GCCACCTCTGACTTGAGCGTCGATTTTTGTGATGCTCGTCAGGGGGGCGGAGCCT |
| | ATGGAAAAACGCCAGCAACGCGGCCTTTTTACGGTTCCTGGCCTTTTGCTGGCCT |
| | TTTGCTCACATGTTCTTTCCTGCGTTATCCCCTGATTCTGTGGATAACCGTATTA |
| | CCGCCTTTGAGTGAGCTGATACCGCTCGCCGCAGCCGAACGACCGAGCGCAGCGA |
| | GTCAGTGAGCGAGGAAGCGGAAGAGCGCCCAATACGCAAACCGCCTCTCCCCGCG |
| | CGTTGGCCGATTCATTAATGCAGCTGCGCGCTCGCTCGCTCACTGAGGCCGCCCG |
| | GGCAAAGCCCGGGCGTCGGGCGACCTTTGGTCGCCCGGCCTCAGTGAGCGAGCGA |
| | GCGCGCAGAGAGGGAGTGGCCAACTCCATCACTAGGGGTTCCTTGTAGTTAATGA |
| | TTAACCCGCCATGCTACTTATCTACGTACCCTATTAAATAAAAGAATAAGCAGTA |
| | TTATTAAGTAGCCCTGCATTTCAGGTTTCCTTGAGTGGCAGGCCAGGCCTGGCCG |
| | TGAACGTTCACTGAAATCATGGCCTCTTGGCCAAGATTGATAGCTTGTGCCTGTC |
| | CCTGAGTCCCAGTCCATCACGAGCAGCTGGTTTCTAAGATGCTATTTCCCGTATA |
| | AAGCATGAGACCGTGACTTGCCAGCCCCACAGAGCCCCGCCCTTGTCCATCACTG |
| | GCATCTGGACTCCAGCCTGGGTTGGGGCAAAGAGGGAAATGAGATCATGTCCTAC |
| | GCGTAGGAACAGAGAAACAGGAGAATATGGGCCAAACAGGATATCTGTGGTAAGC |
| | AGTTCCTGCCCCGGCTCAGGGCCAAGAACAGTTGGAACAGCAGAATATGGGCCAA |
| | ACAGGATATCTGTGGTAAGCAGTTCCTGCCCCGGCTCAGGGCCAAGAACAGATGG |
| | TCCCCAGATGCGGTCCCGCCCTCAGCAGTTTCTAGAGAACCATCAGATGTTTCCA |
| | GGGTGCCCCAAGGACCTGAAATGACCCTGTGCCTTATTTGAACTAACCAATCAGT |
| | TCGCTTCTCGCTTCTGTTCGCGCGCTTCTGCTCCCCGAGCTCTATATAAGCAGAG |
| | CTCGTTTAGTGAACCGTCAGATCGCCTGGAGACGCCATCCACGCTGTTTTGACTT |
| | CCATAGAAGGATCATGGCTCTGCCAGTGACAGCTCTGCTGCTTCCTCTGGCTCTG |
| | TTGCTGCATGCCGCCAGACCTATTCTGTGGCACGAGATGTGGCATGAAGGCCTGG |
| | AAGAGGCCTCCAGACTGTACTTCGGCGAGAGAAACGTGAAGGGCATGTTCGAGGT |
| | GCTGGAACCCCTGCATGCCATGATGGAAAGAGGCCCTCAGACACTGAAAGAGACA |
| | AGCTTCAACCAGGCCTACGGCCGGGATCTGATGGAAGCCCAAGAGTGGTGCCGGA |
| | AGTACATGAAGTCCGGCAATGTGAAGGACCTCCTGCAGGCATGGGACCTGTACTA |
| | CCACGTGTTCCGGCGGATCTCTAAGGGCAAAGACACGATTCCGTGGCTTGGGCAT |
| | CTGCTCGTTGGGCTGAGTGGTGCGTTTGGTTTCATCATCTTGGTCTATCTCTTGA |
| | TCAATTGCAGAAATACAGGCCCTTGGCTGAAAAAAGTGCTCAAGTGTAATACCCC |
| | CGACCCAAGCAAGTTCTTCTCCCAGCTTTCTTCAGAGCATGGAGGCGATGTGCAG |
| | AAATGGCTCTCTTCACCTTTTCCCTCCTCAAGTTTCTCCCCGGGAGGGCTGGCGC |
| | CCGAGATTTCACCTCTTGAGGTACTTGAACGAGACAAGGTTACCCAACTTCTCCT |
| | TCAACAGGATAAAGTACCCGAACCTGCGAGCCTTAGCTCCAACCACTCTCTTACG |
| | AGCTGCTTCACCAATCAGGGATACTTCTTTTTCCACCTTCCCGATGCGCTGGAAA |
| | TCGAAGCATGTCAAGTTTACTTTACCTATGATCCATATAGCGAGGAAGATCCCGA |
| | CGAAGGAGTCGCCGGTGCGCCCACGGGTTCCTCACCCCAACCTCTCCAGCCTCTC |
| | TCAGGAGAAGATGATGCTTATTGCACTTTTCCCAGTAGAGACGATCTCCTCCTCT |
| | TTTCTCCATCTCTTTTGGGGGGACCTTCCCCCCCTTCTACGGCACCTGGCGGGTC |
| | TGGTGCTGGCGAGGAGCGGATGCCGCCGTCCCTCCAGGAGCGAGTACCACGAGAT |
| | TGGGATCCCCAGCCACTTGGACCCCCCACCCCCGGCGTACCTGACCTTGTCGATT |
| | TTCAACCTCCCCCTGAATTGGTGCTGCGAGAGGCTGGGGAGGAAGTTCCGGACGC |
| | TGGGCCGAGGGAGGGCGTGTCCTTTCCATGGAGTAGGCCTCCAGGTCAAGGCGAG |
| | TTTAGGGCTCTCAACGCGCGGCTGCCGTTGAATACAGACGCTTATCTCTCACTGC |
| | AGGAACTGCAAGGTCAGGACCCAACACATCTTGTAGGATCCGGCGCCACCAACTT |
| | TAGTCTGCTCAAGCAAGCCGGGGACGTCGAGGAAAATCCTGGGCCAATGGGATCA |
| | AGACTGCTGTGCTGGGTGCTGCTGTGCCTGCTGGGAGCCGGACCTGTGAAAGCCG |
| | GGGTGACTCAGACTCCACGATACCTGATCAAGACCAGGGGCCAGCAGGTGACACT |
| | GTCTTGCAGCCCAATCTCCGGACACCGCTCCGTGTCTTGGTACCAGCAGACCCCT |
| | GGACAGGGACTGCAGTTCCTGTTTGAGTATTTCTCTGAGACACAGCGGAACAAGG |
| | GCAATTTCCCCGGCCGGTTTAGCGGCAGACAGTTTAGCAACTCCAGGTCTGAGAT |
| | GAATGTGAGCACCCTGGAGCTGGGCGACTCCGCCCTGTACCTGTGCGCAAGCTCC |
| | CTGGCCCTGGGACAGGGGAATCAGCAGTTTTTCGGACCCGGAACAAGGCTGACCG |
| | TGCTGGAGGATCTACGTAACGTGACACCACCCAAAGTCTCACTGTTTGAGCCTAG |
| | CAAGGCAGAAATTGCCAACAAGCAGAAGGCCACCCTGGTGTGCCTGGCAAGAGGG |
| | TTCTTTCCAGATCACGTGGAGCTGTCCTGGTGGGTCAACGGCAAAGAAGTGCATT |
| | CTGGGGTCTGCACCGACCCCCAGGCTTACAAGGAGAGTAATTACTCATATTGTCT |
| | GTCAAGCCGGCTGAGAGTGTCCGCCACATTCTGGCACAACCCTAGGAATCATTTC |
| | CGCTGCCAGGTCCAGTTTCACGGCCTGAGTGAGGAAGATAAATGGCCAGAGGGGT |
| | CACCTAAGCCAGTGACACAGAACATCAGCGCAGAAGCCTGGGGACGAGCAGACTG |
| | TGGCATTACTAGCGCCTCCTATCATCAGGGCGTGCTGAGCGCCACTATCCTGTAC |
| | GAGATTCTGCTGGGAAAGGCCACCCTGTATGCTGTGCTGGTCTCCGGCCTGGTGC |
| | TGATGGCCATGGTCAAGAAAAAGAACTCTGGGAGTGGAGCCACAAATTTCTCTCT |
| | GCTGAAACAGGCTGGAGATGTGGAGGAAAACCCCGGCCCTATGAAGAGCCTGCGC |
| | GTGCTGCTGGTCATCCTGTGGCTGCAATTGAGTTGGGTGTGGAGCCAGAAGGACC |
| | AGGTGTTTCAGCCTAGCACTGTCGCATCATCAGAGGGGGCCGTCGTGGAAATCTT |
| | TTGTAATCACAGCGTGTCCAACGCCTACAATTTCTTTTGGTATCTGCACTTCCCA |
| | GGATGCGCACCTAGGCTGCTGGTGAAGGGCTCTAAGCCCAGCCAGCAGGGCCGGT |
| | ACAACATGACCTATGAGCGGTTCAGCTCCTCTCTGCTGATCCTGCAGGTGCGGGA |
| | GGCAGACGCAGCCGTGTACTATTGTGCCGTGGAGGATCTGAATCAGGCCGGCACA |
| | GCCCTGATTTTCGGCAAAGGAACAACCCTGAGCGTGAGCAGCATCCAGAACCCTG |
| | ACCCAGCCGTATACCAGCTGAGAGACTCTAAATCCAGTGACAAGTCTGTCTGCCT |
| | ATTCACCGATTTTGATTCTCAAACAAATGTGTCACAAAGTAAGGATTCTGATGTG |
| | TATATCACAGACAAAACTGTGCTAGACATGAGGTCTATGGACTTCAAGAGCAACA |
| | GTGCTGTGGCCTGGAGCAACAAATCTGACTTTGCATGTGCAAACGCCTTCAACAA |
| | CAGCATTATTCCAGAAGACACCTTCTTCCCCAGCCCAGGTAAGGGCAGCTTTGGT |
| | GCCTTCGCAGGCTGTTTCCTTGCTTCAGGAATGGATCCTACGTAGATAAGTAGCA |
| | TGGGGGGTTAATCATTAACTACAAGGAACCCCTAGTGATGGAGTTGGCCACTCCC |
| | TCTCTGCGCGCTCGCTCGCTCACTGAGGCCGGGCGACCAAAGGTCGCCCGACGCC |
| | CGGGCTTTGCCCGGGCGGCCTCAGTGAGCGAGCGAGCGCGCCAGCTGGCGTAATA |
| | GCGAAGAGGCCCGCACCGATCGCCCTTCCCAACAGTTGCGCAGCCTGAATGGCGA |
| | ATGGCGATTCCGTTGCAATGGCTGGCGGTAATATTGTTCTGGATATTACCAGCAA |
| | GGCCGATAGTTTGAGTTCTTCTACTCAGGCAAGTGATGTTATTACTAATCAAAGA |
| | AGTATTGCGACAACGGTTAATTTGCGTGATGGACAGACTCTTTTACTCGGTGGCC |
| | TCACTGATTATAAAAACACTTCTCAGGATTCTGGCGTACCGTTCCTGTCTAAAAT |
| | CCCTTTAATCGGCCTCCTGTTTAGCTCCCGCTCTGATTCTAACGAGGAAAGCACG |
| | TTATACGTGCTCGTCAAAGCAACCATAGTACGCGCCCTGTAGCGGCGCATTAAGC |
| | GCGGCGGGTGTGGTGGTTACGCGCAGCGTGACCGCTACACTTGCCAGCGCCCTAG |
| | CGCCCGCTCCTTTCGCTTTCTTCCCTTCCTTTCTCGCCACGTTCGCCGGCTTTCC |
| | CCGTCAAGCTCTAAATCGGGGGCTCCCTTTAGGGTTCCGATTTAGTGCTTTACGG |
| | CACCTCGACCCCAAAAAACTTGATTAGGGTGATGGTTCACGTAGTGGGCCATCGC |
| | CCTGATAGACGGTTTTTCGCCCTTTGACGTTGGAGTCCACGTTCTTTAATAGTGG |
| | ACTCTTGTTCCAAACTGGAACAACACTCAACCCTATCTCGGTCTATTCTTTTGAT |
| | TTATAAGGGATTTTGCCGATTTCGGCCTATTGGTTAAAAAATGAGCTGATTTAAC |
| | AAAAATTTAACGCGAATTTTAACAAAATATTAACGTTTACAATTTAAATATTTGC |
| | TTATACAATCTTCCTGTTTTTGGGGCTTTTCTGATTATCAACCGGGGTACATATG |
| | ATTGACATGCTAGTTTTACGATTACCGTTCATCGATTCTCTTGTTTGCTCCAGAC |
| | TCTCAGGCAATGACCTGATAGCCTTTGTAGAGACCTCTCAAAAATAGCTACCCTC |
| | TCCGGCATGAATTTATCAGCTAGAACGGTTGAATATCATATTGATGGTGATTTGA |
| | CTGTCTCCGGCCTTTCTCACCCGTTTGAATCTTTACCTACACATTACTCAGGCAT |
| | TGCATTTAAAATATATGAGGGTTCTAAAAATTTTTATCCTTGCGTTGAAATAAAG |
| | GCTTCTCCCGCAAAAGTATTACAGGGTCATAATGTTTTTGGTACAACCGATTTAG |
| | CTTTATGCTCTGAGGCTTTATTGQTTAATTTTGCTAATTCTTTGCCTTGCCTGTA |
| | TGATTTATTGGATGTTGGAATCGCCTGATGCGGTATTTTCTCCTTACGCATCTGT |
| | GCGGTATTTCACACCGCATATGGTGCACTCTCAGTACAATCTGCTCTGATGCCGC |
| | ATAGTTAAGCCAGCCCCGACACCCGCCAACACCCGCTGACGCGCCCTGACGGGCT |
| | TGTCTGCTCCCGGCATCCGCTTACAGACAAGCTGTGACCGTCTCCGGGAGCTGCA |
| | TGTGTCAGAGGTTTTCACCGTCATCACCGAAACGCGCGAGACGAAAGGGCCTCGT |
| | GATACGCCTATTTTTATAGGTTAATGTCATGATAATAATGGTTTCTTAGACGTCA |
| | GGTGGCACTTTTCGGGGAAATGTGCGCGGAACCCCTATTTGTTTATTTTTCTAAA |
| | TACATTCAAATATGTATCCGCTCATGAGACAATAACCCTGATAAATGCTTCAATA |
| | ATATTGAAAAAGGAAGAGTATGAGTATTCAACATTTCCGTGTCGCCCTTATTCCC |
| | TTTTTTGCGGCATTTTGCCTTCCTGTTTTTGCTCACCCAGAAACGCTGGTGAAAG |
| | TAAAAGATGCTGAAGATCAGTTGGGTGCACGAGTGGGTTACATCGAACTGGATCT |
| | CAACAGCGGTAAGATCCTTGAGAGTTTTCGCCCCGAAGAACGTTTTCCAATGATG |
| | AGCACTTTTAAAGTTCTGCTATGTGGCGCGGTATTATCCCGTATTGACGCCGGGC |
| | AAGAGCAACTCGGTCGCCGCATACACTATTCTCAGAATGACTTGGTTGAGTACTC |
| | ACCAGTCACAGAAAAGCATCTTACGGATGGCATGACAGTAAGAGAATTATGCAGT |
| | GCTGCCATAACCATGAGTGATAACACTGCGGCCAACTTACTTCTGACAACGATCG |
| | GAGGACCGAAGGAGCTAACCGCTTTTTTGCACAACATGGGGGATCATGTAACTCG |
| | CCTTGATCGTTGGGAACCGGAGCTGAATGAAGCCATACCAAACGACGAGCGTGAC |
| | ACCACGATGCCTGTAGCAATGGCAACAACGTTGCGCAAACTATTAACTGGCGAAC |
| | TACTTACTCTAGCTTCCCGGCAACAATTAATAGACTGGATGGAGGCGGATAAAGT |
| | TGCAGGACCACTTCTGCGCTCGGCCCTTCCGGCTGGCTGGTTTATTGCTGATAAA |
| | TCTGGAGCCGGTGAGCGTGGGTCTCGCGGTATCATTGCAGCACTGGGGCCAGATG |
| | GTAAGCCCTCCCGTATCGTAGTTATCTACACGACGGGGAGTCAGGCAACTATGGA |
| | TGAACGAAATAGACAGATCGCTGAGATAGGTGCCTCACTGATTAAGCATTGGTAA |
| | CTGTCAGACCAAGTTTACTCATATATACTTTAGATTGATTTAAAACTTCATTTTT |
| | AATTTAAAAGGATCTAGGTGAAGATCCTTTTTGATAATCTCATGACCAAAATCCC |
| | TTAACGTGAGTTTTCGTTCCACTGAGCGTCAGACCCC |
| Construct | GTAGAAAAGATCAAAGGATCTTCTTGAGATCCTTTTTTTCTGCGCGTAATCTGCT |
| 3362 | GCTTGCAAACAAAAAAACCACCGCTACCAGCGGTGGTTTGTTTGCCGGATCAAGA |
| SEQ ID | GCTACCAACTCTTTTTCCGAAGGTAACTGGCTTCAGCAGAGCGCAGATACCAAAT |
| NO: 1447 | ACTGTCCTTCTAGTGTAGCCGTAGTTAGGCCACCACTTCAAGAACTCTGTAGCAC |
| | CGCCTACATACCTCGCTCTGCTAATCCTGTTACCAGTGGCTGCTGCCAGTGGCGA |
| | TAAGTCGTGTCTTACCGGGTTGGACTCAAGACGATAGTTACCGGATAAGGCGCAG |
| | CGGTCGGGCTGAACGGGGGGTTCGTGCACACAGCCCAGCTTGGAGCGAACGACCT |
| | ACACCGAACTGAGATACCTACAGCGTGAGCTATGAGAAAGCGCCACGCTTCCCGA |
| | AGGGAGAAAGGCGGACAGGTATCCGGTAAGCGGCAGGGTCGGAACAGGAGAGCGC |
| | ACGAGGGAGCTTCCAGGGGGAAACGCCTGGTATCTTTATAGTCCTGTCGGGTTTC |
| | GCCACCTCTGACTTGAGCGTCGATTTTTGTGATGCTCGTCAGGGGGGCGGAGCCT |
| | ATGGAAAAACGCCAGCAACGCGGCCTTTTTACGGTTCCTGGCCTTTTGCTGGCCT |
| | TTTGCTCACATGTTCTTTCCTGCGTTATCCCCTGATTCTGTGGATAACCGTATTA |
| | CCGCCTTTGAGTGAGCTGATACCGCTCGCCGCAGCCGAACGACCGAGCGCAGCGA |
| | GTCAGTGAGCGAGGAAGCGGAAGAGCGCCCAATACGCAAACCGCCTCTCCCCGCG |
| | CGTTGGCCGATTCATTAATGCAGCTGCGCGCTCGCTCGCTCACTGAGGCCGCCCG |
| | GGCAAAGCCCGGGCGTCGGGCGACCTTTGGTCGCCCGGCCTCAGTGAGCGAGCGA |
| | GCGCGCAGAGAGGGAGTGGCCAACTCCATCACTAGGGGTTCCTTGTAGTTAATGA |
| | TTAACCCGCCATGCTACTTATCTACGTAGCGGCCGCAGCCTGTGCAGGGTGCAGG |
| | GAGGGCTAGAGGCCTGAGGCTTGAAACAGCTCTCAAGTGGAGGGGGAAACAACCA |
| | TTGCCCTCATAGAGGACACATCCACACCAGGGCTGTGCTAGCGTGGGCAGGCAAG |
| | CCAGGTGCTGGACCTCTGCACGTGGGGCATGTGTGGGTATGTACATGTACCTGTG |
| | TTCTTGGTGTGTGTGTGTGTGTGTGTGTGTGTGTGTGTCTAGAGCTGGGGTGCAA |
| | CTATGGGGCCCCTCGGGACATGTCCCAGCCAATGCCTGCTTTGACCAGAGGAGTG |
| | TCCACGTGGCTCAGGTGGTCGAGTATCTCATACCGCCCTAGCACACGTGTGACTC |
| | CTTTCCCCTATTGTCTACGCAGCCTGCCCTTGGACAAGGACCCGATGCCCAACCC |
| | CAGGCCTGGCAAGCCCTCGGCCCCTTCCTTGGCCCTTGGCCCATCCCCACGCGTA |
| | GGAACAGAGAAACAGGAGAATATGGGCCAAACAGGATATCTGTGGTAAGCAGTTC |
| | CTGCCCCGGCTCAGGGCCAAGAACAGTTGGAACAGCAGAATATGGGCCAAACAGG |
| | ATATCTGTGGTAAGCAGTTCCTGCCCCGGCTCAGGGCCAAGAACAGATGGTCCCC |
| | AGATGCGGTCCCGCCCTCAGCAGTTTCTAGAGAACCATCAGATGTTTCCAGGGTG |
| | CCCCAAGGACCTGAAATGACCCTGTGCCTTATTTGAACTAACCAATCAGTTCGCT |
| | TCTCGCTTCTGTTCGCGCGCTTCTGCTCCCCGAGCTCTATATAAGCAGAGCTCGT |
| | TTAGTGAACCGTCAGATCGCCTGGAGACGCCATCCACGCTGTTTTGACTTCCATA |
| | GAAGGATCTCGAGGCCACCATGCCTCTGGGCCTGCTGTGGCTGGGCCTGGCCCTG |
| | CTGGGCGCCCTGCACGCCCAGGCCGAGATGTGGCACGAGGGCCTGGAGGAGGCCA |
| | GCAGGCTGTATTTTGGCGAGCGCAACGTGAAGGGCATGTTCGAGGTGCTGGAGCC |
| | TCTGCACGCCATGATGGAGAGAGGCCCACAGACCCTGAAGGAGACATCCTTTAAC |
| | CAGGCCTATGGACGGGACCTGATGGAGGCACAGGAGTGGTGCAGAAAGTACATGA |
| | AGTCTGGCAATGTGAAGGACCTGCTGCAGGCCTGGGATCTGTACTATCACGTGTT |
| | TCGGAGAATCTCCAAGGGCAAAGACACGATTCCGTGGCTTGGGCATCTGCTCGTT |
| | GGGCTGAGTGGTGCGTTTGGTTTCATCATCTTGGTCTATCTCTTGATCAATTGCA |
| | GAAATACAGGCCCTTGGCTGAAAAAAGTGCTCAAGTGTAATACCCCCGACCCAAG |
| | CAAGTTCTTCTCCCAGCTTTCTTCAGAGCATGGAGGCGATGTGCAGAAATGGCTC |
| | TCTTCACCTTTTCCCTCCTCAAGTTTCTCCCCGGGAGGGCTGGCGCCCGAGATTT |
| | CACCTCTTGAGGTACTTGAACGAGACAAGGTTACCCAACTTCTCCTTCAACAGGA |
| | TAAGGTACCCGAACCTGCGAGCCTTAGCTCCAACCACTCTCTTACGAGCTGCTTC |
| | ACCAATCAGGGATACTTCTTTTTCCACCTTCCCGATGCGCTGGAAATCGAAGCAT |
| | GTCAAGTTTACTTTACCTATGATCCATATAGCGAGGAAGATCCCGACGAAGGAGT |
| | CGCCGGTGCGCCCACGGGTTCCTCACCCCAACCTCTCCAGCCTCTCTCAGGAGAA |
| | GATGATGCTTATTGCACTTTTCCCAGTAGAGACGATCTCCTCCTCTTTTCTCCAT |
| | CTCTTTTGGGGGGACCTTCCCCCCCTTCTACGGCACCTGGCGGGTCTGGTGCTGG |
| | CGAGGAGCGGATGCCGCCGTCCCTCCAGGAGCGAGTACCACGAGATTGGGATCCC |
| | CAGCCACTTGGACCACCAACTCCAGGCGTACCTGACCTTGTCGATTTTCAACCTC |
| | CCCCTGAATTGGTGCTGCGAGAGGCTGGGGAGGAAGTTCCGGACGCTGGGCCGAG |
| | GGAGGGCGTGTCCTTTCCATGGAGTAGGCCTCCAGGTCAAGGCGAGTTTAGGGCT |
| | CTCAACGCGCGGCTGCCGTTGAATACAGACGCTTATCTCTCACTGCAGGAACTGC |
| | AAGGTCAGGACCCAACACATCTTGTAGGATCCGGCGCCACCAACTTTAGTCTGCT |
| | CAAGCAAGCCGGGGACGTCGAGGAAAATCCTGGGCCAGAAATGTGGCACGAAGGA |
| | CTCGAGGAAGCCAGTCGGCTGTATTTTGGCGAGCGGAATGTGAAAGGGATGTTTG |
| | AAGTGCTCGAGCCTCTCCACGCTATGATGGAACGGGGACCCCAGACTCTCAAAGA |
| | AACCAGCTTTAATCAGGCTTACGGACGCGACCTCATGGAAGCTCAAGAATGGTGT |
| | AGAAAGTATATGAAGAGTGGCAACGTGAAAGATCTGCTGCAAGCCTGGGATCTCT |
| | ATTATCACGTGTTCAGACGCATCAGCAAAGGCAGCGGCGCCACAAATTTCTCCCT |
| | GCTGAAACAGGCCGGCGACGTGGAAGAGAATCCCGGACCTTATCCATACGATGTC |
| | CCAGATTATGCGCCCAATCCTAGACCTGGCAAGCCCAGCGCTCCTTCTCTTGCTC |
| | TTGGACCTTCTCCTGGTGCCTCGCCCAGCTGGAGGGCTGCACCCAAAGCCTCAGA |
| | CCTGCTGGGGGCCCGGGGCCCAGGGGGAACCTTCCAGGGCCGAGATCTTCGAGGC |
| | GGGGCCCATGCCTCCTCTTCTTCCTTGAACCCCATGCCACCATCGCAGCTGCAGG |
| | TGAGGCCCTGGGCCCAGGATGGGGCAGGCAGGGTGGGGTACCTGGACCTACAGGT |
| | GCCGACCTTTACTGTGGCACTGGGCGGGAGGGGGGCTGGCTGGGGCACAGGAAGT |
| | GGTTTCTGGGTCCCAGGCAAGTCTGTGACTTATGCAGATGTTGCAGGGCCAAGAA |
| | AATCCCCACCTGCCAGGCCTCAGAGATTGGAGGCTCTCCCCGACCTCCCAATCCC |
| | TGTCTCAGGAGAGGAGGAGGCCGTATTGTAGTCCCATGAGCATAGCTATGTGTCC |
| | CCATCCCCATGTGACAAGAGAAGAGGAGGATCCTACGTAGATAAGTAGCATGGCG |
| | GGTTAATCATTAACTACAAGGAACCCCTAGTGATGGAGTTGGCCACTCCCTCTCT |
| | GCGCGCTCGCTCGCTCACTGAGGCCGGGCGACCAAAGGTCGCCCGACGCCCGGGC |
| | TTTGCCCGGGCGGCCTCAGTGAGCGAGCGAGCGCGCCAGCTGGCGTAATAGCGAA |
| | GAGGCCCGCACCGATCGCCCTTCCCAACAGTTGCGCAGCCTGAATGGCGAATGGC |
| | GATTCCGTTGCAATGGCTGGCGGTAATATTGTTCTGGATATTACCAGCAAGGCCG |
| | ATAGTTTGAGTTCTTCTACTCAGGCAAGTGATGTTATTACTAATCAAAGAAGTAT |
| | TGCGACAACGGTTAATTTGCGTGATGGACAGACTCTTTTACTCGGTGGCCTCACT |
| | GATTATAAAAACACTTCTCAGGATTCTGGCGTACCGTTCCTGTCTAAAATCCCTT |
| | TAATCGGCCTCCTGTTTAGCTCCCGCTCTGATTCTAACGAGGAAAGCACGTTATA |
| | CGTGCTCGTCAAAGCAACCATAGTACGCGCCCTGTAGCGGCGCATTAAGCGCGGC |
| | GGGTGTGGTGGTTACGCGCAGCGTGACCGCTACACTTGCCAGCGCCCTAGCGCCC |
| | GCTCCTTTCGCTTTCTTCCCTTCCTTTCTCGCCACGTTCGCCGGCTTTCCCCGTC |
| | AAGCTCTAAATCGGGGGCTCCCTTTAGGGTTCCGATTTAGTGCTTTACGGCACCT |
| | CGACCCCAAAAAACTTGATTAGGGTGATGGTTCACGTAGTGGGCCATCGCCCTGA |
| | TAGACGGTTTTTCGCCCTTTGACGTTGGAGTCCACGTTCTTTAATAGTGGACTCT |
| | TGTTCCAAACTGGAACAACACTCAACCCTATCTCGGTCTATTCTTTTGATTTATA |
| | AGGGATTTTGCCGATTTCGGCCTATTGGTTAAAAAATGAGCTGATTTAACAAAAA |
| | TTTAACGCGAATTTTAACAAAATATTAACGTTTACAATTTAAATATTTGCTTATA |
| | CAATCTTCCTGTTTTTGGGGCTTTTCTGATTATCAACCGGGGTACATATGATTGA |
| | CATGCTAGTTTTACGATTACCGTTCATCGATTCTCTTGTTTGCTCCAGACTCTCA |
| | GGCAATGACCTGATAGCCTTTGTAGAGACCTCTCAAAAATAGCTACCCTCTCCGG |
| | CATGAATTTATCAGCTAGAACGGTTGAATATCATATTGATGGTGATTTGACTGTC |
| | TCCGGCCTTTCTCACCCGTTTGAATCTTTACCTACACATTACTCAGGCATTGCAT |
| | TTAAAATATATGAGGGTTCTAAAAATTTTTATCCTTGCGTTGAAATAAAGGCTTC |
| | TCCCGCAAAAGTATTACAGGGTCATAATGTTTTTGGTACAACCGATTTAGCTTTA |
| | TGCTCTGAGGCTTTATTGCTTAATTTTGCTAATTCTTTGCCTTGCCTGTATGATT |
| | TATTGGATGTTGGAATCGCCTGATGCGGTATTTTCTCCTTACGCATCTGTGCGGT |
| | ATTTCACACCGCATATGGTGCACTCTCAGTACAATCTGCTCTGATGCCGCATAGT |
| | TAAGCCAGCCCCGACACCCGCCAACACCCGCTGACGCGCCCTGACGGGCTTGTCT |
| | GCTCCCGGCATCCGCTTACAGACAAGCTGTGACCGTCTCCGGGAGCTGCATGTGT |
| | CAGAGGTTTTCACCGTCATCACCGAAACGCGCGAGACGAAAGGGCCTCGTGATAC |
| | GCCTATTTTTATAGGTTAATGTCATGATAATAATGGTTTCTTAGACGTCAGGTGG |
| | CACTTTTCGGGGAAATGTGCGCGGAACCCCTATTTGTTTATTTTTCTAAATACAT |
| | TCAAATATGTATCCGCTCATGAGACAATAACCCTGATAAATGCTTCAATAATATT |
| | GAAAAAGGAAGAGTATGAGTATTCAACATTTCCGTGTCGCCCTTATTCCCTTTTT |
| | TGCGGCATTTTGCCTTCCTGTTTTTGCTCACCCAGAAACGCTGGTGAAAGTAAAA |
| | GATGCTGAAGATCAGTTGGGTGCACGAGTGGGTTACATCGAACTGGATCTCAACA |
| | GCGGTAAGATCCTTGAGAGTTTTCGCCCCGAAGAACGTTTTCCAATGATGAGCAC |
| | TTTTAAAGTTCTGCTATGTGGCGCGGTATTATCCCGTATTGACGCCGGGCAAGAG |
| | CAACTCGGTCGCCGCATACACTATTCTCAGAATGACTTGGTTGAGTACTCACCAG |
| | TCACAGAAAAGCATCTTACGGATGGCATGACAGTAAGAGAATTATGCAGTGCTGC |
| | CATAACCATGAGTGATAACACTGCGGCCAACTTACTTCTGACAACGATCGGAGGA |
| | CCGAAGGAGCTAACCGCTTTTTTGCACAACATGGGGGATCATGTAACTCGCCTTG |
| | ATCGTTGGGAACCGGAGCTGAATGAAGCCATACCAAACGACGAGCGTGACACCAC |
| | GATGCCTGTAGCAATGGCAACAACGTTGCGCAAACTATTAACTGGCGAACTACTT |
| | ACTCTAGCTTCCCGGCAACAATTAATAGACTGGATGGAGGCGGATAAAGTTGCAG |
| | GACCACTTCTGCGCTCGGCCCTTCCGGCTGGCTGGTTTATTGCTGATAAATCTGG |
| | AGCCGGTGAGCGTGGGTCTCGCGGTATCATTGCAGCACTGGGGCCAGATGGTAAG |
| | CCCTCCCGTATCGTAGTTATCTACACGACGGGGAGTCAGGCAACTATGGATGAAC |
| | GAAATAGACAGATCGCTGAGATAGGTGCCTCACTGATTAAGCATTGGTAACTGTC |
| | AGACCAAGTTTACTCATATATACTTTAGATTGATTTAAAACTTCATTTTTAATTT |
| | AAAAGGATCTAGGTGAAGATCCTTTTTGATAATCTCATGACCAAAATCCCTTAAC |
| | GTGAGTTTTCGTTCCACTGAGCGTCAGACCCC |
| Construct | GTAGAAAAGATCAAAGGATCTTCTTGAGATCCTTTTTTTCTGCGCGTAATCTGCT |
| 3363 | GCTTGCAAACAAAAAAACCACCGCTACCAGCGGTGGTTTGTTTGCCGGATCAAGA |
| SEQ ID | GCTACCAACTCTTTTTCCGAAGGTAACTGGCTTCAGCAGAGCGCAGATACCAAAT |
| NO: 1448 | ACTGTCCTTCTAGTGTAGCCGTAGTTAGGCCACCACTTCAAGAACTCTGTAGCAC |
| | CGCCTACATACCTCGCTCTGCTAATCCTGTTACCAGTGGCTGCTGCCAGTGGCGA |
| | TAAGTCGTGTCTTACCGGGTTGGACTCAAGACGATAGTTACCGGATAAGGCGCAG |
| | CGGTCGGGCTGAACGGGGGGTTCGTGCACACAGCCCAGCTTGGAGCGAACGACCT |
| | ACACCGAACTGAGATACCTACAGCGTGAGCTATGAGAAAGCGCCACGCTTCCCGA |
| | AGGGAGAAAGGCGGACAGGTATCCGGTAAGCGGCAGGGTCGGAACAGGAGAGCGC |
| | ACGAGGGAGCTTCCAGGGGGAAACGCCTGGTATCTTTATAGTCCTGTCGGGTTTC |
| | GCCACCTCTGACTTGAGCGTCGATTTTTGTGATGCTCGTCAGGGGGGCGGAGCCT |
| | ATGGAAAAACGCCAGCAACGCGGCCTTTTTACGGTTCCTGGCCTTTTGCTGGCCT |
| | TTTGCTCACATGTTCTTTCCTGCGTTATCCCCTGATTCTGTGGATAACCGTATTA |
| | CCGCCTTTGAGTGAGCTGATACCGCTCGCCGCAGCCGAACGACCGAGCGCAGCGA |
| | GTCAGTGAGCGAGGAAGCGGAAGAGCGCCCAATACGCAAACCGCCTCTCCCCGCG |
| | CGTTGGCCGATTCATTAATGCAGCTGCGCGCTCGCTCGCTCACTGAGGCCGCCCG |
| | GGCAAAGCCCGGGCGTCGGGCGACCTTTGGTCGCCCGGCCTCAGTGAGCGAGCGA |
| | GCGCGCAGAGAGGGAGTGGCCAACTCCATCACTAGGGGTTCCTTGTAGTTAATGA |
| | TTAACCCGCCATGCTACTTATCTACGTACCCTATTAAATAAAAGAATAAGCAGTA |
| | TTATTAAGTAGCCCTGCATTTCAGGTTTCCTTGAGTGGCAGGCCAGGCCTGGCCG |
| | TGAACGTTCACTGAAATCATGGCCTCTTGGCCAAGATTGATAGCTTGTGCCTGTC |
| | CCTGAGTCCCAGTCCATCACGAGCAGCTGGTTTCTAAGATGCTATTTCCCGTATA |
| | AAGCATGAGACCGTGACTTGCCAGCCCCACAGAGCCCCGCCCTTGTCCATCACTG |
| | GCATCTGGACTCCAGCCTGGGTTGGGGCAAAGAGGGAAATGAGATCATGTCCTAC |
| | GCGTAGGAACAGAGAAACAGGAGAATATGGGCCAAACAGGATATCTGTGGTAAGC |
| | AGTTCCTGCCCCGGCTCAGGGCCAAGAACAGTTGGAACAGCAGAATATGGGCCAA |
| | ACAGGATATCTGTGGTAAGCAGTTCCTGCCCCGGCTCAGGGCCAAGAACAGATGG |
| | TCCCCAGATGCGGTCCCGCCCTCAGCAGTTTCTAGAGAACCATCAGATGTTTCCA |
| | GGGTGCCCCAAGGACCTGAAATGACCCTGTGCCTTATTTGAACTAACCAATCAGT |
| | TCGCTTCTCGCTTCTGTTCGCGCGCTTCTGCTCCCCGAGCTCTATATAAGCAGAG |
| | CTCGTTTAGTGAACCGTCAGATCGCCTGGAGACGCCATCCACGCTGTTTTGACTT |
| | CCATAGAAGGATCATGCCTCTGGGCCTGCTGTGGCTGGGCCTGGCCCTGCTGGGC |
| | GCCCTGCACGCCCAGGCCGGCGTGCAGGTGGAGACAATCTCCCCAGGCGACGGAC |
| | GCACATTCCCTAAGCGGGGCCAGACCTGCGTGGTGCACTATACAGGCATGCTGGA |
| | GGATGGCAAGAAGTTTGACAGCTCCCGGGATAGAAACAAGCCATTCAAGTTTATG |
| | CTGGGCAAGCAGGAAGTGATCAGAGGCTGGGAGGAGGGCGTGGCCCAGATGTCTG |
| | TGGGCCAGAGGGCCAAGCTGACCATCAGCCCAGACTACGCCTATGGAGCAACAGG |
| | CCACCCAGGAATCATCCCACCTCACGCCACCCTGGTGTTCGATGTGGAGCTGCTG |
| | AAGCTGGGCGAGGGATCCAACACATCAAAAGAGAACCCCTTTCTGTTCGCATTGG |
| | AGGCCGTAGTCATATCTGTTGGATCCATGGGACTTATTATCTCCCTGTTGTGTGT |
| | GTACTTCTGGCTGGAACGGACTATGCCCAGGATCCCCACGCTCAAGAATCTGGAA |
| | GATCTCGTCACAGAATACCATGGTAATTTCAGCGCCTGGAGCGGAGTCTCTAAGG |
| | GTCTGGCCGAATCCCTCCAACCCGATTATTCTGAACGGTTGTGCCTCGTATCCGA |
| | AATACCACCAAAAGGGGGGCTCTGGGTGAGGGCCCAGGGGCGAGTCCGTGCAATC |
| | AACACAGCCCGTATTGGGCCCCTCCTTGTTATACGTTGAAGCCCGAAACTGGATC |
| | CGGCGCCACCAACTTTAGTCTGCTCAAGCAAGCCGGGGACGTCGAGGAAAATCCT |
| | GGGCCAATGGGATCAAGACTGCTGTGCTGGGTGCTGCTGTGCCTGCTGGGAGCCG |
| | GACCTGTGAAAGCCGGGGTGACTCAGACTCCACGATACCTGATCAAGACCAGGGG |
| | CCAGCAGGTGACACTGTCTTGCAGCCCAATCTCCGGACACCGCTCCGTGTCTTGG |
| | TACCAGCAGACCCCTGGACAGGGACTGCAGTTCCTGTTTGAGTATTTCTCTGAGA |
| | CACAGCGGAACAAGGGCAATTTCCCCGGCCGGTTTAGCGGCAGACAGTTTAGCAA |
| | CTCCAGGTCTGAGATGAATGTGAGCACCCTGGAGCTGGGCGACTCCGCCCTGTAC |
| | CTGTGCGCAAGCTCCCTGGCCCTGGGACAGGGGAATCAGCAGTTTTTCGGACCCG |
| | GAACAAGGCTGACCGTGCTGGAGGATCTACGTAACGTGACACCACCCAAAGTCTC |
| | ACTGTTTGAGCCTAGCAAGGCAGAAATTGCCAACAAGCAGAAGGCCACCCTGGTG |
| | TGCCTGGCAAGAGGGTTCTTTCCAGATCACGTGGAGCTGTCCTGGTGGGTCAACG |
| | GCAAAGAAGTGCATTCTGGGGTCTGCACCGACCCCCAGGCTTACAAGGAGAGTAA |
| | TTACTCATATTGTCTGTCAAGCCGGCTGAGAGTGTCCGCCACATTCTGGCACAAC |
| | CCTAGGAATCATTTCCGCTGCCAGGTCCAGTTTCACGGCCTGAGTGAGGAAGATA |
| | AATGGCCAGAGGGGTCACCTAAGCCAGTGACACAGAACATCAGCGCAGAAGCCTG |
| | GGGACGAGCAGACTGTGGCATTACTAGCGCCTCCTATCATCAGGGCGTGCTGAGC |
| | GCCACTATCCTGTACGAGATTCTGCTGGGAAAGGCCACCCTGTATGCTGTGCTGG |
| | TCTCCGGCCTGGTGCTGATGGCCATGGTCAAGAAAAAGAACTCTGGGAGTGGAGC |
| | CACAAATTTCTCTCTGCTGAAACAGGCTGGAGATGTGGAGGAAAACCCCGGCCCT |
| | ATGAAGAGCCTGCGCGTGCTGCTGGTCATCCTGTGGCTGCAATTGAGTTGGGTGT |
| | GGAGCCAGAAGGACCAGGTGTTTCAGCCTAGCACTGTCGCATCATCAGAGGGGGC |
| | CGTCGTGGAAATCTTTTGTAATCACAGCGTGTCCAACGCCTACAATTTCTTTTGG |
| | TATCTGCACTTCCCAGGATGCGCACCTAGGCTGCTGGTGAAGGGCTCTAAGCCCA |
| | GCCAGCAGGGCCGGTACAACATGACCTATGAGCGGTTCAGCTCCTCTCTGCTGAT |
| | CCTGCAGGTGCGGGAGGCAGACGCAGCCGTGTACTATTGTGCCGTGGAGGATCTG |
| | AATCAGGCCGGCACAGCCCTGATTTTCGGCAAAGGAACAACCCTGAGCGTGAGCA |
| | GCATCCAGAACCCTGACCCAGCCGTATACCAGCTGAGAGACTCTAAATCCAGTGA |
| | CAAGTCTGTCTGCCTATTCACCGATTTTGATTCTCAAACAAATGTGTCACAAAGT |
| | AAGGATTCTGATGTGTATATCACAGACAAAACTGTGCTAGACATGAGGTCTATGG |
| | ACTTCAAGAGCAACAGTGCTGTGGCCTGGAGCAACAAATCTGACTTTGCATGTGC |
| | AAACGCCTTCAACAACAGCATTATTCCAGAAGACACCTTCTTCCCCAGCCCAGGT |
| | AAGGGCAGCTTTGGTGCCTTCGCAGGCTGTTTCCTTGCTTCAGGAATGGATCCTA |
| | CGTAGATAAGTAGCATGGCGGGTTAATCATTAACTACAAGGAACCCCTAGTGATG |
| | GAGTTGGCCACTCCCTCTCTGCGCGCTCGCTCGCTCACTGAGGCCGGGCGACCAA |
| | AGGTCGCCCGACGCCCGGGCTTTGCCCGGGCGGCCTCAGTGAGCGAGCGAGCGCG |
| | CCAGCTGGCGTAATAGCGAAGAGGCCCGCACCGATCGCCCTTCCCAACAGTTGCG |
| | CAGCCTGAATGGCGAATGGCGATTCCGTTGCAATGGCTGGCGGTAATATTGTTCT |
| | GGATATTACCAGCAAGGCCGATAGTTTGAGTTCTTCTACTCAGGCAAGTGATGTT |
| | ATTACTAATCAAAGAAGTATTGCGACAACGGTTAATTTGCGTGATGGACAGACTC |
| | TTTTACTCGGTGGCCTCACTGATTATAAAAACACTTCTCAGGATTCTGGCGTACC |
| | GTTCCTGTCTAAAATCCCTTTAATCGGCCTCCTGTTTAGCTCCCGCTCTGATTCT |
| | AACGAGGAAAGCACGTTATACGTGCTCGTCAAAGCAACCATAGTACGCGCCCTGT |
| | AGCGGCGCATTAAGCGCGGCGGGTGTGGTGGTTACGCGCAGCGTGACCGCTACAC |
| | TTGCCAGCGCCCTAGCGCCCGCTCCTTTCGCTTTCTTCCCTTCCTTTCTCGCCAC |
| | GTTCGCCGGCTTTCCCCGTCAAGCTCTAAATCGGGGGCTCCCTTTAGGGTTCCGA |
| | TTTAGTGCTTTACGGCACCTCGACCCCAAAAAACTTGATTAGGGTGATGGTTCAC |
| | GTAGTGGGCCATCGCCCTGATAGACGGTTTTTCGCCCTTTGACGTTGGAGTCCAC |
| | GTTCTTTAATAGTGGACTCTTGTTCCAAACTGGAACAACACTCAACCCTATCTCG |
| | GTCTATTCTTTTGATTTATAAGGGATTTTGCCGATTTCGGCCTATTGGTTAAAAA |
| | ATGAGCTGATTTAACAAAAATTTAACGCGAATTTTAACAAAATATTAACGTTTAC |
| | AATTTAAATATTTGCTTATACAATCTTCCTGTTTTTGGGGCTTTTCTGATTATCA |
| | ACCGGGGTACATATGATTGACATGCTAGTTTTACGATTACCGTTCATCGATTCTC |
| | TTGTTTGCTCCAGACTCTCAGGCAATGACCTGATAGCCTTTGTAGAGACCTCTCA |
| | AAAATAGCTACCCTCTCCGGCATGAATTTATCAGCTAGAACGGTTGAATATCATA |
| | TTGATGGTGATTTGACTGTCTCCGGCCTTTCTCACCCGTTTGAATCTTTACCTAC |
| | ACATTACTCAGGCATTGCATTTAAAATATATGAGGGTTCTAAAAATTTTTATCCT |
| | TGCGTTGAAATAAAGGCTTCTCCCGCAAAAGTATTACAGGGTCATAATGTTTTTG |
| | GTACAACCGATTTAGCTTTATGCTCTGAGGCTTTATTGCTTAATTTTGCTAATTC |
| | TTTGCCTTGCCTGTATGATTTATTGGATGTTGGAATCGCCTGATGCGGTATTTTC |
| | TCCTTACGCATCTGTGCGGTATTTCACACCGCATATGGTGCACTCTCAGTACAAT |
| | CTGCTCTGATGCCGCATAGTTAAGCCAGCCCCGACACCCGCCAACACCCGCTGAC |
| | GCGCCCTGACGGGCTTGTCTGCTCCCGGCATCCGCTTACAGACAAGCTGTGACCG |
| | TCTCCGGGAGCTGCATGTGTCAGAGGTTTTCACCGTCATCACCGAAACGCGCGAG |
| | ACGAAAGGGCCTCGTGATACGCCTATTTTTATAGGTTAATGTCATGATAATAATG |
| | GTTTCTTAGACGTCAGGTGGCACTTTTCGGGGAAATGTGCGCGGAACCCCTATTT |
| | GTTTATTTTTCTAAATACATTCAAATATGTATCCGCTCATGAGACAATAACCCTG |
| | ATAAATGCTTCAATAATATTGAAAAAGGAAGAGTATGAGTATTCAACATTTCCGT |
| | GTCGCCCTTATTCCCTTTTTTGCGGCATTTTGCCTTCCTGTTTTTGCTCACCCAG |
| | AAACGCTGGTGAAAGTAAAAGATGCTGAAGATCAGTTGGGTGCACGAGTGGGTTA |
| | CATCGAACTGGATCTCAACAGCGGTAAGATCCTTGAGAGTTTTCGCCCCGAAGAA |
| | CGTTTTCCAATGATGAGCACTTTTAAAGTTCTGCTATGTGGCGCGGTATTATCCC |
| | GTATTGACGCCGGGCAAGAGCAACTCGGTCGCCGCATACACTATTCTCAGAATGA |
| | CTTGGTTGAGTACTCACCAGTCACAGAAAAGCATCTTACGGATGGCATGACAGTA |
| | AGAGAATTATGCAGTGCTGCCATAACCATGAGTGATAACACTGCGGCCAACTTAC |
| | TTCTGACAACGATCGGAGGACCGAAGGAGCTAACCGCTTTTTTGCACAACATGGG |
| | GGATCATGTAACTCGCCTTGATCGTTGGGAACCGGAGCTGAATGAAGCCATACCA |
| | AACGACGAGCGTGACACCACGATGCCTGTAGCAATGGCAACAACGTTGCGCAAAC |
| | TATTAACTGGCGAACTACTTACTCTAGCTTCCCGGCAACAATTAATAGACTGGAT |
| | GGAGGCGGATAAAGTTGCAGGACCACTTCTGCGCTCGGCCCTTCCGGCTGGCTGG |
| | TTTATTGCTGATAAATCTGGAGCCGGTGAGCGTGGGTCTCGCGGTATCATTGCAG |
| | CACTGGGGCCAGATGGTAAGCCCTCCCGTATCGTAGTTATCTACACGACGGGGAG |
| | TCAGGCAACTATGGATGAACGAAATAGACAGATCGCTGAGATAGGTGCCTCACTG |
| | ATTAAGCATTGGTAACTGTCAGACCAAGTTTACTCATATATACTTTAGATTGATT |
| | TAAAACTTCATTTTTAATTTAAAAGGATCTAGGTGAAGATCCTTTTTGATAATCT |
| | CATGACCAAAATCCCTTAACGTGAGTTTTCGTTCCACTGAGCGTCAGACCCC |
| Construct | GTAGAAAAGATCAAAGGATCTTCTTGAGATCCTTTTTTTCTGCGCGTAATCTGCT |
| 3364 | GCTTGCAAACAAAAAAACCACCGCTACCAGCGGTGGTTTGTTTGCCGGATCAAGA |
| SEQ ID | GCTACCAACTCTTTTTCCGAAGGTAACTGGCTTCAGCAGAGCGCAGATACCAAAT |
| NO: 1449 | ACTGTCCTTCTAGTGTAGCCGTAGTTAGGCCACCACTTCAAGAACTCTGTAGCAC |
| | CGCCTACATACCTCGCTCTGCTAATCCTGTTACCAGTGGCTGCTGCCAGTGGCGA |
| | TAAGTCGTGTCTTACCGGGTTGGACTCAAGACGATAGTTACCGGATAAGGCGCAG |
| | CGGTCGGGCTGAACGGGGGGTTCGTGCACACAGCCCAGCTTGGAGCGAACGACCT |
| | ACACCGAACTGAGATACCTACAGCGTGAGCTATGAGAAAGCGCCACGCTTCCCGA |
| | AGGGAGAAAGGCGGACAGGTATCCGGTAAGCGGCAGGGTCGGAACAGGAGAGCGC |
| | ACGAGGGAGCTTCCAGGGGGAAACGCCTGGTATCTTTATAGTCCTGTCGGGTTTC |
| | GCCACCTCTGACTTGAGCGTCGATTTTTGTGATGCTCGTCAGGGGGGCGGAGCCT |
| | ATGGAAAAACGCCAGCAACGCGGCCTTTTTACGGTTCCTGGCCTTTTGCTGGCCT |
| | TTTGCTCACATGTTCTTTCCTGCGTTATCCCCTGATTCTGTGGATAACCGTATTA |
| | CCGCCTTTGAGTGAGCTGATACCGCTCGCCGCAGCCGAACGACCGAGCGCAGCGA |
| | GTCAGTGAGCGAGGAAGCGGAAGAGCGCCCAATACGCAAACCGCCTCTCCCCGCG |
| | CGTTGGCCGATTCATTAATGCAGCTGCGCGCTCGCTCGCTCACTGAGGCCGCCCG |
| | GGCAAAGCCCGGGCGTCGGGCGACCTTTGGTCGCCCGGCCTCAGTGAGCGAGCGA |
| | GCGCGCAGAGAGGGAGTGGCCAACTCCATCACTAGGGGTTCCTTGTAGTTAATGA |
| | TTAACCCGCCATGCTACTTATCTACGTAGCGGCCGCACTGAAATCATGGCCTCTT |
| | GGCCAAGATTGATAGCTTGTGCCTGTCCCTGAGTCCCAGTCCATCACGAGCAGCT |
| | GGTTTCTAAGATGCTATTTCCCGTATAAAGCATGAGACCGTGACTTGCCAGCCCC |
| | ACAGAGCCCCGCCCTTGTCCATCACTGGCATCTGGACTCCAGCCTGGGTTGGGGC |
| | AAAGAGGGAAATGAGATCATGTCCTAACCCTGATCCTCTTGTCCCACAGATATCC |
| | AGAACCCTGACCCTGCCGTGTACCAGCTGAGAGACTCTAAATCCAGTGACAAGTC |
| | TGTCTGCCTATATTAAGGAACAGAGAAACAGGAGAATATGGGCCAAACAGGATAT |
| | CTGTGGTAAGCAGTTCCTGCCCCGGCTCAGGGCCAAGAACAGTTGGAACAGCAGA |
| | ATATGGGCCAAACAGGATATCTGTGGTAAGCAGTTCCTGCCCCGGCTCAGGGCCA |
| | AGAACAGATGGTCCCCAGATGCGGTCCCGCCCTCAGCAGTTTCTAGAGAACCATC |
| | AGATGTTTCCAGGGTGCCCCAAGGACCTGAAATGACCCTGTGCCTTATTTGAACT |
| | AACCAATCAGTTCGCTTCTCGCTTCTGTTCGCGCGCTTCTGCTCCCCGAGCTCTA |
| | TATAAGCAGAGCTCGTTTAGTGAACCGTCAGATCGCCTGGAGACGCCATCCACGC |
| | TGTTTTGACTTCCATAGAAGGATCGACTCGAGTAATGCCTCTGGGCCTGCTGTGG |
| | CTGGGCCTGGCCCTGCTGGGCGCCCTGCACGCCCAGGCCGGCGTGCAGGTGGAGA |
| | CAATCTCCCCAGGCGACGGACGCACATTCCCTAAGCGGGGCCAGACCTGCGTGGT |
| | GCACTATACAGGCATGCTGGAGGATGGCAAGAAGTTTGACAGCTCCCGGGATAGA |
| | AACAAGCCATTCAAGTTTATGCTGGGCAAGCAGGAAGTGATCAGAGGCTGGGAGG |
| | AGGGCGTGGCCCAGATGTCTGTGGGCCAGAGGGCCAAGCTGACCATCAGCCCAGA |
| | CTACGCCTATGGAGCAACAGGCCACCCAGGAATCATCCCACCTCACGCCACCCTG |
| | GTGTTCGATGTGGAGCTGCTGAAGCTGGGCGAGGGATCCAACACATCAAAAGAGA |
| | ACCCCTTTCTGTTCGCATTGGAGGCCGTAGTCATATCTGTTGGATCCATGGGACT |
| | TATTATCTCCCTGTTGTGTGTGTACTTCTGGCTGGAACGGACTATGCCCAGGATC |
| | CCCACGCTCAAGAATCTGGAAGATCTCGTCACAGAATACCATGGTAATTTCAGCG |
| | CCTGGAGCGGAGTCTCTAAGGGTCTGGCCGAATCCCTCCAACCCGATTATTCTGA |
| | ACGGTTGTGCCTCGTATCCGAAATACCACCAAAAGGCGGGGCTCTGGGTGAGGGC |
| | CCAGGGGCGAGTCCGTGCAATCAACACAGCCCGTATTGGGCCCCTCCTTGTTATA |
| | CGTTGAAGCCCGAAACTGGCTCCGGCGCCACCAACTTTAGTCTGCTCAAGCAAGC |
| | CGGGGACGTCGAGGAAAATCCTGGGCCAATGGGATCAAGACTGCTGTGCTGGGTG |
| | CTGCTGTGCCTGCTGGGAGCCGGACCTGTGAAAGCCGGGGTGACTCAGACTCCAC |
| | GATACCTGATCAAGACCAGGGGCCAGCAGGTGACACTGTCTTGCAGCCCAATCTC |
| | CGGACACCGCTCCGTGTCTTGGTACCAGCAGACCCCTGGACAGGGACTGCAGTTC |
| | CTGTTTGAGTATTTCTCTGAGACACAGCGGAACAAGGGCAATTTCCCCGGCCGGT |
| | TTAGCGGCAGACAGTTTAGCAACTCCAGGTCTGAGATGAATGTGAGCACCCTGGA |
| | GCTGGGCGACTCCGCCCTGTACCTGTGCGCAAGCTCCCTGGCCCTGGGACAGGGG |
| | AATCAGCAGTTTTTCGGACCCGGAACAAGGCTGACCGTGCTGGAGGATCTACGTA |
| | ACGTGACACCACCCAAAGTCTCACTGTTTGAGCCTAGCAAGGCAGAAATTGCCAA |
| | CAAGCAGAAGGCCACCCTGGTGTGCCTGGCAAGAGGGTTCTTTCCAGATCACGTG |
| | GAGCTGTCCTGGTGGGTCAACGGCAAAGAAGTGCATTCTGGGGTCTGCACCGACC |
| | CCCAGGCTTACAAGGAGAGTAATTACTCATATTGTCTGTCAAGCCGGCTGAGAGT |
| | GTCCGCCACATTCTGGCACAACCCTAGGAATCATTTCCGCTGCCAGGTCCAGTTT |
| | CACGGCCTGAGTGAGGAAGATAAATGGCCAGAGGGGTCACCTAAGCCAGTGACAC |
| | AGAACATCAGCGCAGAAGCCTGGGGACGAGCAGACTGTGGCATTACTAGCGCCTC |
| | CTATCATCAGGGCGTGCTGAGCGCCACTATCCTGTACGAGATTCTGCTGGGAAAG |
| | GCCACCCTGTATGCTGTGCTGGTCTCCGGCCTGGTGCTGATGGCCATGGTCAAGA |
| | AAAAGAACTCTGGGAGTGGAGCCACAAATTTCTCTCTGCTGAAACAGGCTGGAGA |
| | TGTGGAGGAAAACCCCGGCCCTATGAAGAGCCTGCGCGTGCTGCTGGTCATCCTG |
| | TGGCTGCAATTGAGTTGGGTGTGGAGCCAGAAGGACCAGGTGTTTCAGCCTAGCA |
| | CTGTCGCATCATCAGAGGGGGCCGTCGTGGAAATCTTTTGTAATCACAGCGTGTC |
| | CAACGCCTACAATTTCTTTTGGTATCTGCACTTCCCAGGATGCGCACCTAGGCTG |
| | CTGGTGAAGGGCTCTAAGCCCAGCCAGCAGGGCCGGTACAACATGACCTATGAGC |
| | GGTTCAGCTCCTCTCTGCTGATCCTGCAGGTGCGGGAGGCAGACGCAGCCGTGTA |
| | CTATTGTGCCGTGGAGGATCTGAATCAGGCCGGCACAGCCCTGATTTTCGGCAAA |
| | GGAACAACCCTGAGCGTGAGCAGCGACATCCAGAACCCTGAGCCCGCCGTATACC |
| | AGCTGAAGGACCCCCGATCTCAGGATAGTACTCTGTGCCTGTTCACCGACTTTGA |
| | TAGTCAGATCAATGTGCCTAAAACCATGGAATCCGGAACTTTTATTACCGACAAG |
| | TGCGTGCTGGATATGAAAGCCATGGACAGTAAGTCAAACGGCGCCATCGCTTGGA |
| | GCAATCAGACATCCTTCACTTGCCAGGATATCTTCAAGGAGACCAACGCAACATA |
| | CCCATCCTCTGACGTGCCCTGTGATGCCACCCTGACAGAGAAGTCTTTCGAAACA |
| | GACATGAACCTGAATTTTCAGAATCTGAGCGTGATGGGCCTGAGAATCCTGCTGC |
| | TGAAGGTCGCTGGGTTTAATCTGCTGATGACACTGCGGCTGTGGTCCTCATAAAG |
| | CTTAATTGAGCGGCCGCGCTTTATTTGTGAAATTTGTGATGCTATTGCTTTATTT |
| | GTAACCATTATAAGCTGCAATAAACAAGTTAACAACAACAATTGCATTCATTTTA |
| | TGTTTCAGGTTCAGGGGGAGATGTGGGAGGTTTTTTAAAGCTCACCGATTTTGAT |
| | TCTCAAACAAATGTGTCACAAAGTAAGGATTCTGATGTGTATATCACAGACAAAA |
| | CTGTGCTAGACATGAGGTCTATGGACTTCAAGAGCAACAGTGCTGTGGCCTGGAG |
| | CAACAAATCTGACTTTGCATGTGCAAACGCCTTCAACAACAGCATTATTCCAGAA |
| | GACACCTTCTTCCCCAGCCCAGGTAAGGGCAGCTTTGGTGCCTTCGCAGGCTGTT |
| | TCCTTGCTTCAGGAATGGCCAGGTTCTGCCCAGAGCTCTGGTCAATGATGTCTAA |
| | AACTCCTCTGATTGGTGGTCTCGGCGCGGCCGCTACGTAGATAAGTAGCATGGCG |
| | GGTTAATCATTAACTACAAGGAACCCCTAGTGATGGAGTTGGCCACTCCCTCTCT |
| | GCGCGCTCGCTCGCTCACTGAGGCCGGGCGACCAAAGGTCGCCCGACGCCCGGGC |
| | TTTGCCCGGGCGGCCTCAGTGAGCGAGCGAGCGCGCCAGCTGGCGTAATAGCGAA |
| | GAGGCCCGCACCGATCGCCCTTCCCAACAGTTGCGCAGCCTGAATGGCGAATGGC |
| | GATTCCGTTGCAATGGCTGGCGGTAATATTGTTCTGGATATTACCAGCAAGGCCG |
| | ATAGTTTGAGTTCTTCTACTCAGGCAAGTGATGTTATTACTAATCAAAGAAGTAT |
| | TGCGACAACGGTTAATTTGCGTGATGGACAGACTCTTTTACTCGGTGGCCTCACT |
| | GATTATAAAAACACTTCTCAGGATTCTGGCGTACCGTTCCTGTCTAAAATCCCTT |
| | TAATCGGCCTCCTGTTTAGCTCCCGCTCTGATTCTAACGAGGAAAGCACGTTATA |
| | CGTGCTCGTCAAAGCAACCATAGTACGCGCCCTGTAGCGGCGCATTAAGCGCGGC |
| | GGGTGTGGTGGTTACGCGCAGCGTGACCGCTACACTTGCCAGCGCCCTAGCGCCC |
| | GCTCCTTTCGCTTTCTTCCCTTCCTTTCTCGCCACGTTCGCCGGCTTTCCCCGTC |
| | AAGCTCTAAATCGGGGGCTCCCTTTAGGGTTCCGATTTAGTGCTTTACGGCACCT |
| | CGACCCCAAAAAACTTGATTAGGGTGATGGTTCACGTAGTGGGCCATCGCCCTGA |
| | TAGACGGTTTTTCGCCCTTTGACGTTGGAGTCCACGTTCTTTAATAGTGGACTCT |
| | TGTTCCAAACTGGAACAACACTCAACCCTATCTCGGTCTATTCTTTTGATTTATA |
| | AGGGATTTTGCCGATTTCGGCCTATTGGTTAAAAAATGAGCTGATTTAACAAAAA |
| | TTTAACGCGAATTTTAACAAAATATTAACGTTTACAATTTAAATATTTGCTTATA |
| | CAATCTTCCTGTTTTTGGGGCTTTTCTGATTATCAACCGGGGTACATATGATTGA |
| | CATGCTAGTTTTACGATTACCGTTCATCGATTCTCTTGTTTGCTCCAGACTCTCA |
| | GGCAATGACCTGATAGCCTTTGTAGAGACCTCTCAAAAATAGCTACCCTCTCCGG |
| | CATGAATTTATCAGCTAGAACGGTTGAATATCATATTGATGGTGATTTGACTGTC |
| | TCCGGCCTTTCTCACCCGTTTGAATCTTTACCTACACATTACTCAGGCATTGCAT |
| | TTAAAATATATGAGGGTTCTAAAAATTTTTATCCTTGCGTTGAAATAAAGGCTTC |
| | TCCCGCAAAAGTATTACAGGGTCATAATGTTTTTGGTACAACCGATTTAGCTTTA |
| | TGCTCTGAGGCTTTATTGCTTAATTTTGCTAATTCTTTGCCTTGCCTGTATGATT |
| | TATTGGATGTTGGAATCGCCTGATGCGGTATTTTCTCCTTACGCATCTGTGCGGT |
| | ATTTCACACCGCATATGGTGCACTCTCAGTACAATCTGCTCTGATGCCGCATAGT |
| | TAAGCCAGCCCCGACACCCGCCAACACCCGCTGACGCGCCCTGACGGGCTTGTCT |
| | GCTCCCGGCATCCGQTTACAGACAAGCTGTGACCGTCTCCGGGAGCTGCATGTGT |
| | CAGAGGTTTTCACCGTCATCACCGAAACGCGCGAGACGAAAGGGCCTCGTGATAC |
| | GCCTATTTTTATAGGTTAATGTCATGATAATAATGGTTTCTTAGACGTCAGGTGG |
| | CACTTTTCGGGGAAATGTGCGCGGAACCCCTATTTGTTTATTTTTCTAAATACAT |
| | TCAAATATGTATCCGCTCATGAGACAATAACCCTGATAAATGCTTCAATAATATT |
| | GAAAAAGGAAGAGTATGAGTATTCAACATTTCCGTGTCGCCCTTATTCCCTTTTT |
| | TGCGGCATTTTGCCTTCCTGTTTTTGCTCACCCAGAAACGCTGGTGAAAGTAAAA |
| | GATGCTGAAGATCAGTTGGGTGCACGAGTGGGTTACATCGAACTGGATCTCAACA |
| | GCGGTAAGATCCTTGAGAGTTTTCGCCCCGAAGAACGTTTTCCAATGATGAGCAC |
| | TTTTAAAGTTCTGCTATGTGGCGCGGTATTATCCCGTATTGACGCCGGGCAAGAG |
| | CAACTCGGTCGCCGCATACACTATTCTCAGAATGACTTGGTTGAGTACTCACCAG |
| | TCACAGAAAAGCATCTTACGGATGGCATGACAGTAAGAGAATTATGCAGTGCTGC |
| | CATAACCATGAGTGATAACACTGCGGCCAACTTACTTCTGACAACGATCGGAGGA |
| | CCGAAGGAGCTAACCGCTTTTTTGCACAACATGGGGGATCATGTAACTCGCCTTG |
| | ATCGTTGGGAACCGGAGCTGAATGAAGCCATACCAAACGACGAGCGTGACACCAC |
| | GATGCCTGTAGCAATGGCAACAACGTTGCGCAAACTATTAACTGGCGAACTACTT |
| | ACTCTAGCTTCCCGGCAACAATTAATAGACTGGATGGAGGCGGATAAAGTTGCAG |
| | GACCACTTCTGCGCTCGGCCCTTCCGGCTGGCTGGTTTATTGCTGATAAATCTGG |
| | AGCCGGTGAGCGTGGGTCTCGCGGTATCATTGCAGCACTGGGGCCAGATGGTAAG |
| | CCCTCCCGTATCGTAGTTATCTACACGACGGGGAGTCAGGCAACTATGGATGAAC |
| | GAAATAGACAGATCGCTGAGATAGGTGCCTCACTGATTAAGCATTGGTAACTGTC |
| | AGACCAAGTTTACTCATATATACTTTAGATTGATTTAAAACTTCATTTTTAATTT |
| | AAAAGGATCTAGGTGAAGATCCTTTTTGATAATCTCATGACCAAAATCCCTTAAC |
| | GTGAGTTTTCGTTCCACTGAGCGTCAGACCCC |
| naked FRB | EMWHEGLEEASRLYFGERNVKGMFEVLEPLHAMMERGPQTLKETSFNQAYGRDLM |
| wild-type | EAQEWCRKYMKSGNVKDLTQAWDLYYHVFRRISK |
| polypeptide | |
| SEQ ID | |
| NO: 1450 | |
| FRB-CISC | GAGATGTGGCACGAGGGCCTGGAGGAGGCCAGCAGGCTGTATTTTGGCGAGCGCA |
| having a | ACGTGAAGGGCATGTTCGAGGTGCTGGAGCCTCTGCACGCCATGATGGAGAGAGG |
| intracellular | CCCACAGACCCTGAAGGAGACATCCTTTAACCAGGCCTATGGACGGGACCTGATG |
| IL2Rβ | GAGGCACAGGAGTGGTGCAGAAAGTACATGAAGTCTGGCAATGTGAAGGACCTGA |
| domain | CGCAGGCCTGGGATCTGTACTATCACGTGTTTCGGAGAATCTCCAAGGGCAAAGA |
| SEQ ID | CACGATTCCGTGGCTTGGGCATCTGCTCGTTGGGCTGAGTGGTGCGTTTGGTTTC |
| NO: 1451 | ATCATCTTGGTCTATCTCTTGATCAATTGCAGAAATACAGGCCCTTGGCTGAAAA |
| | AAGTGCTCAAGTGTAATACCCCCGACCCAAGCAAGTTCTTCTCCCAGCTTTCTTC |
| | AGAGCATGGAGGCGATGTGCAGAAATGGCTCTCTTCACCTTTTCCCTCCTCAAGT |
| | TTCTCCCCGGGAGGGCTGGCGCCCGAGATTTCACCTCTTGAGGTACTTGAACGAG |
| | ACAAGGTTACCCAACTTCTCCTTCAACAGGATAAGGTACCCGAACCTGCGAGCCT |
| | TAGCTCCAACCACTCTCTTACGAGCTGCTTCACCAATCAGGGATACTTCTTTTTC |
| | CACCTTCCCGATGCGCTGGAAATCGAAGCATGTCAAGTTTACTTTACCTATGATC |
| | CATATAGCGAGGAAGATCCCGACGAAGGAGTCGCCGGTGCGCCCACGGGTTCCTC |
| | ACCCCAACCTCTCCAGCCTCTCTCAGGAGAAGATGATGCTTATTGCACTTTTCCC |
| | AGTAGAGACGATCTCCTCCTCTTTTCTCCATCTCTTTTGGGGGGACCTTCCCCCC |
| | CTTCTACGGCACCTGGCGGGTCTGGTGCTGGCGAGGAGCGGATGCCGCCGTCCCT |
| | CCAGGAGCGAGTACCACGAGATTGGGATCCCCAGCCACTTGGACCACCAACTCCA |
| | GGCGTACCTGACCTTGTCGATTTTCAACCTCCCCCTGAATTGGTGCTGCGAGAGG |
| | CTGGGGAGGAAGTTCCGGACGCTGGGCCGAGGGAGGGCGTGTCCTTTCCATGGAG |
| | TAGGCCTCCAGGTCAAGGCGAGTTTAGGGCTCTCAACGCGCGGCTGCCGTTGAAT |
| | ACAGACGCTTATCTCTCACTGCAGGAACTGCAAGGTCAGGACCCAACACATCTTG |
| | TA |
| FRB | GAGATGTGGCACGAGGGCCTGGAGGAGGCCAGCAGGCTGTATTTTGGCGAGCGCA |
| domain of | ACGTGAAGGGCATGTTCGAGGTGCTGGAGCCTCTGCACGCCATGATGGAGAGAGG |
| polypeptide | CCCACAGACCCTGAAGGAGACATCCTTTAACCAGGCCTATGGACGGGACCTGATG |
| encoded by | GAGGCACAGGAGTGGTGCAGAAAGTACATGAAGTCTGGCAATGTGAAGGACCTGA |
| SEQ | CGCAGGCCTGGGATCTGTACTATCACGTGTTTCGGAGAATCTCCAAG |
| ID: 1451 | |
| SEQ ID | |
| NO: 1452 | |
| IL2Rβ | GGCAAAGACACG |
| domain of | |
| polypeptide | |
| encoded by | |
| SEQ | |
| ID: 1451 | |
| SEQ ID | |
| NO: 1453 | |
| Transmembrane | ATTCCGTGGCTTGGGCATCTGCTCGTTGGGCTGAGTGGTGCGTTTGGTTTCATCA |
| domain of | TCTTGGTCTATCTCTTGATC |
| polypeptide | |
| encoded by | |
| SEQ | |
| ID: 1451 | |
| SEQ ID | |
| NO: 1454 | |
| Intracellular | AATTGCAGAAATACAGGCCCTTGGCTGAAAAAAGTGCTCAAGTGTAATACCCCCG |
| domain of | ACCCAAGCAAGTTCTTCTCCCAGCTTTCTTCAGAGCATGGAGGCGATGTGCAGAA |
| polypeptide | ATGGCTCTCTTCACCTTTTCCCTCCTCAAGTTTCTCCCCGGGAGGGCTGGCGCCC |
| encoded by | GAGATTTCACCTCTTGAGGTACTTGAACGAGACAAGGTTACCCAACTTCTCCTTC |
| SEQ | AACAGGATAAGGTACCCGAACCTGCGAGCCTTAGCTCCAACCACTCTCTTACGAG |
| ID: 1451 | CTGCTTCACCAATCAGGGATACTTCTTTTTCCACCTTCCCGATGCGCTGGAAATC |
| SEQ ID | GAAGCATGTCAAGTTTACTTTACCTATGATCCATATAGCGAGGAAGATCCCGACG |
| NO: 1455 | AAGGAGTCGCCGGTGCGCCCACGGGTTCCTCACCCCAACCTCTCCAGCCTCTCTC |
| | AGGAGAAGATGATGCTTATTGCACTTTTCCCAGTAGAGACGATCTCCTCCTCTTT |
| | TCTCCATCTCTTTTGGGGGGACCTTCCCCCCCTTCTACGGCACCTGGCGGGTCTG |
| | GTGCTGGCGAGGAGCGGATGCCGCCGTCCCTCCAGGAGCGAGTACCACGAGATTG |
| | GGATCCCCAGCCACTTGGACCACCAACTCCAGGCGTACCTGACCTTGTCGATTTT |
| | CAACCTCCCCCTGAATTGGTGCTGCGAGAGGCTGGGGAGGAAGTTCCGGACGCTG |
| | GGCCGAGGGAGGGCGTGTCCTTTCCATGGAGTAGGCCTCCAGGTCAAGGCGAGTT |
| | TAGGGCTCTCAACGCGCGGCTGCCGTTGAATACAGACGCTTATCTCTCACTGCAG |
| | GAACTGCAAGGTCAGGACCCAACACATCTTGTA |

### Certain Embodiments for Suppressing Activation and/or Proliferation of a Population of Cells

Some embodiments of the methods and compositions provided herein include methods of suppressing activation and/or proliferation of a population of cells, such as polyclonal T cells. Some such methods include contacting the population of cells with a genetically modified CD4+ Treg cell; wherein the population of cells comprise an endogenous T cell receptor (TCR) specific for a first epitope of an antigen, and wherein the Treg cell comprises an exogenous TCR specific for a second epitope of the antigen. In some embodiments, the method provides administering to a subject one or more airT cells that express a TCR or CAR specific to an epitope of an antigen, wherein the subject comprises polyclonal T effector cells that express TCRs specific to one or more epitopes of the same antigen. In some embodiments, the administered airT cells express a TCR or CAR that binds an epitope of an antigen with lower affinity or avidity than one or more TCRs of the polyclonal T effector cell population. In some embodiments, the administered airT cells express a TCR or CAR that binds an epitope of an antigen with higher affinity or avidity than one or more TCRs of the polyclonal T effector cell population.

In some embodiments, compositions comprising cells, and methods are provided of suppressing a population of cells in a subject, which population of cells comprises an endogenous TCR. In some embodiments, this endogenous TCR is specific for a first epitope of an antigen. Contemplated herein is a cell (e.g., an engineered T cell) that comprises an exogenous TCR specific for a second epitope of the antigen. In some embodiments, a method as contemplated herein comprises administering to a subject a composition comprising cells (e.g., engineered Treg cells) that comprises an exogenous TCR specific for a second epitope of the antigen.

As used herein, “avidity” can refer to a functional activity induced by an interaction between a TCR expressed on a T cell with an antigen, such as a MHC Class II-peptide complex comprising an antigen on an antigen presenting cell. In some embodiment, a relative avidity of a first TCR to an antigen can include the level of proliferation induced by a first cell expressing the first TCR when the first TCR binds to the antigen compared to the level of proliferation induced by a second cell expressing a second TCR when the second TCR binds to the antigen. In some embodiments, a dose response curve and the measurement of proliferation as shown in FIG. 156A, panel A can be utilized to determine relative avidity. There are multiple ways to assess TCR avidity for a MHC-peptide complex. In some embodiments, a functional definition of avidity can be based on the proliferative response of a T cell expressing the TCR of interest when co-cultured with an antigen presenting cell. In some embodiments, the antigen presenting cell and T cell are autologous. In some embodiments, proliferation is measured with dye dilution at 4-6 days. In some embodiments, proliferation is measured with H3-Thymidine incorporation. In some embodiments, proliferation is measured with expression of Ki67. In some embodiments, avidity is determined by comparison to an established TCR known to be of high avidity. In some embodiments, a panel of TCR is used, generated from responses to known pathogens or autoantigens.

In some embodiments, the exogenous TCR has an increased avidity for the antigen compared to an additional or second TCR specific for the antigen. In some embodiment, a relative avidity of a first TCR to an antigen can include the level of activation and/or proliferation induced by a first cell expressing the first TCR when the first TCR binds to the antigen compared to the level of proliferation induced by a second cell expressing a second TCR when the second TCR binds to the antigen. In some embodiments, methods for determining relative avidity include determining relative functional avidity. Some such methods can include methods disclosed in Viganó S., et al., (2012) Journal of Immunology Research Article ID 153863, 14 pages, 2012 which is incorporated by reference in it is entirety. In some embodiments, functional avidity relates to a proliferative response of a T cell expressing the TCR of interest when co-cultured with an antigen presenting cell. Proliferation can be measured in multiple ways including dye dilution at 4-6 days, H³ thymidine incorporation or expression of Ki67. Such assays can be performed using a range of peptide concentrations. Relative avidity can be determined by comparison to an established TCR known to be of high avidity, such as a panel of TCR generated from responses to known pathogens or autoantigens. For example, a T1D5-2 TCR specific to IGRP has higher avidity for its cognate epitope, IGRP₃₀₅₋₃₂₄, than a T1D4 TCR that is also specific to IGRP, but has the cognate epitope IGRP₂₄₁₋₂₆₀. In some embodiments, a cell expressing an exogenous TCR proliferates 10% to 1,000%, 20% to 500%, 50% to 400%, or 100% to 200% more than a cell expressing an endogenous TCR specific to an epitope of the same antigen. In some embodiments, a cell expressing an exogenous TCR proliferates 10% or more, 20% or more, 30% or more, 40% or more, 50% or more, 60% or more, 70% or more, 80% or more, 90% or more, 100% or more, 150% or more, 200% or more, 250% or more, 300% or more, 350% or more, 400% or more, or 500% or more than a cell expressing an endogenous TCR specific to an epitope of the same antigen. In some embodiments, a cell expressing an exogenous TCR expresses 10% to 1,000%, 20% to 500%, 50% to 400%, or 100% to 200% more of a a cytokine than a cell expressing an endogenous TCR specific to an epitope of the same antigen. In some embodiments, a cell expressing an exogenous TCR expresses 10% or more, 20% or more, 30% or more, 40% or more, 50% or more, 60% or more, 70% or more, 80% or more, 90% or more, 100% or more, 150% or more, 200% or more, 250% or more, 300% or more, 350% or more, 400% or more, or 500% or more of a cytokine than a cell expressing an endogenous TCR specific to an epitope of the same antigen.

In some embodiments, the exogenous TCR has a reduced avidity for the antigen compared to an additional or second TCR specific for the antigen. In some embodiments, a cell expressing an exogenous TCR proliferates 0% to 2%, 2% to 5%, 5% to 10%, 10% to 20%, 20% to 30%, 30% to 40%, 40% to 50%, 50% to 60%, 60% to 70%, 70% to 80%, 80% to 90%, or 90% to 99% as much as a cell expressing an endogenous or second TCR specific to a different epitope of the same antigen. In some embodiments, a cell expressing an exogenous TCR proliferates 99% or less, 95% or less, 90% or less, 80% or less, 75% or less, 70% or less, 60% or less, 50% or less, 40% or less, 30% or less, 20% or less, or 10% or less than a cell expressing an endogenous or second TCR specific to a different epitope of the same antigen. In some embodiments, a cell expressing an exogenous TCR expresses 0% to 2%, 2% to 5%, 5% to 10%, 10% to 20%, 20% to 30%, 30% to 40%, 40% to 50%, 50% to 60%, 60% to 70%, 70% to 80%, 80% to 90%, or 90% to 99% as much of a cytokine as a cell expressing an endogenous or second TCR specific to a different epitope of the same antigen. In some embodiments, a cell expressing an exogenous TCR expresses 99% or less, 95% or less, 90% or less, 80% or less, 75% or less, 70% or less, 60% or less, 50% or less, 40% or less, 30% or less, 20% or less, or 10% or less of a cytokine than a cell expressing an endogenous or second TCR specific to a different epitope of the same antigen.

In some embodiments, the population of cells comprises CD4+ CD25− T cells. In some embodiments, the exogenous TCR is specific for a type I diabetes antigen. In some embodiments, the exogenous TCR is specific for a type I diabetes antigen selected from IGRP, GAD65, or PPI. In some embodiments, the exogenous TCR is selected from T1D2, T1D4, T1D5-1, T1D5-2, 4.13, GAD113, or PPI76. In some embodiments, the exogenous TCR comprises T1D5-2. In some embodiments, the population of cells are contacted with the genetically modified Treg cell in the presence of an antigen presenting cell and the antigen. In some embodiments, the Treg cell is obtained by introducing into a cell a vector comprising a nucleic acid encoding the exogenous TCR. In some embodiments, the Treg cell is a mammalian cell. In some embodiments, the Treg cell is a human cell. In some embodiments, the Treg cell is a T regulatory type 1 (Tr1) cell. In some embodiments, the Treg cell is a FOXP3+ Treg cell. In some embodiments, the Treg cell expresses CTLA-4, LAG-3, CD25, CD39, neuropilin-1, galectin-1, and/or IL-2Ra on its surface. In some embodiments, the cell is obtained from peripheral blood. In some embodiments, the cell is obtained from umbilical cord blood. In some embodiments, the cell is an allogeneic cell. In some embodiments, the cell is an autologous cell.

This application is based, at least in part, on the realization that (1) the suppression phenotype of engineered Treg cells to suppress a polyclonal population of Teff cells is different compared to its phenotype to suppress Teff cells that are transduced to encode specific TCRs; and (2) Tregs with low avidity TCRs are better able to suppress a polyclonal population of Teff cells.

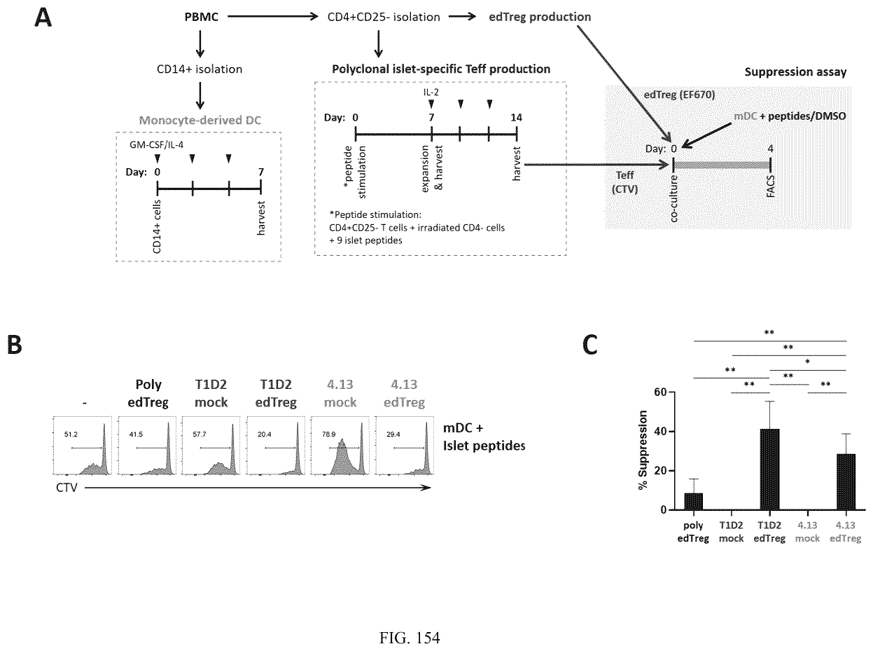

Accordingly, provided herein are methods of preparing a composition of engineered Treg cells wherein the Treg cells suppress a population of polyclonal T effector cells. In some embodiments, a method of preparing a composition of engineered Treg cells able to suppress a population of polyclonal T effector cells comprises (a) contacting the population of Teff cells with a genetically modified CD4+ Treg cell, wherein the population of cells comprise an endogenous T cell receptor (TCR) specific for a first epitope of an antigen, and the Treg cell comprises an exogenous TCR specific for a second epitope of the antigen, and (b) measuring the effect of the Treg cells on the proliferation of the Teff cells as a measure of the suppressive activity of the Treg cells. In some embodiments, the Treg cells and the Teff cells are contacted in the presence of an antigen presenting cell and the antigen. See e.g., FIG. 154, panel A.

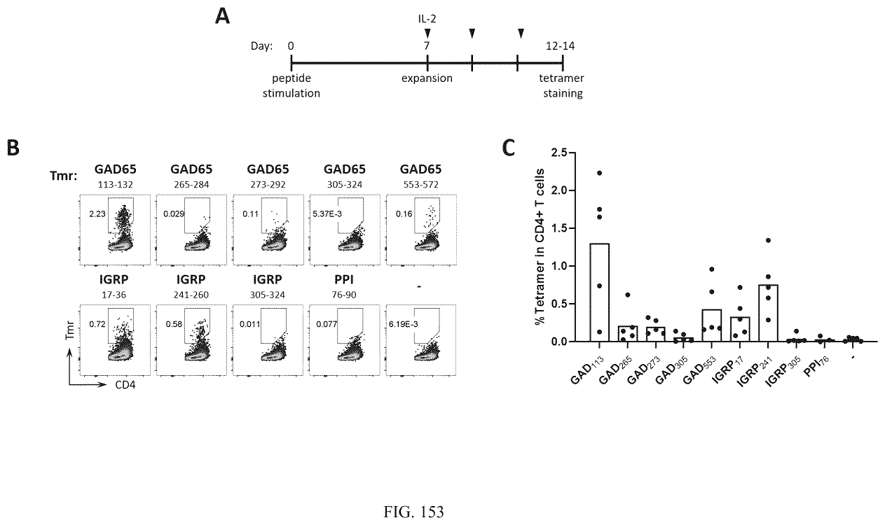

Provided herein is a population of T effector (Teff) cells that can be used to identify engineered Treg cells on the basis of their ability to suppress Teff cells. In some embodiments, Teff cells used to identify Treg cells that can suppress a polyclonal population of Teff cells are prepared by a method comprising: (a) isolating CD4+CD25− T cells from PBMC (e.g., from type 1 diabetes, T1D subjects), and (b) culturing the T cells in the presence of a pool or two or more (e.g., 1, 2, 3, 4, 5, 6, 7, 8, 9, 10, or more than 10) antigen specific peptides (e.g., islet-specific peptides) for a period of time (e.g., 1-30 days, 2-20 days, 5-15 days, 8-15 days, 10-15, days, or 12-15 days). In some embodiments, cultured Teff cells can be tested for their polyclonality using a tetramer staining assay in which specific antigens are loaded on tetramers before binding to cells (see e.g., FIG. 153, panel C)

Provided herein is a composition of engineered Treg cells capable of suppressing a population of polyclonal T effector cells. In some embodiments, an engineered Treg capable of suppressing a population of polyclonal T effector cells has TCR with a low functional avidity. In some embodiments, the avidity of a TCR is measured by the % proliferation of the cell expressing it in response to the antigen to which the TCR is specific (cognate antigen). See e.g., FIG. 156A, panels A and B, and FIG. 156B. In some embodiments, the functional avidity of a TCR is low if the % proliferation of the cell expressing the TCR in response to contact with at least 1 μg/mL (e.g., 1 μg/mL, 2 μg/mL, 3 μg/mL, 4 μg/mL, or 5 μg/mL) of antigen is less than 50% (e.g., less than 50%, less than 40%, less than 30%, less than 20%, or less than 10%). In some embodiments, functional avidity is defined as the antigen dose that is needed to trigger a T-cell response and can be measured by determining the level/s of cytokine production (e.g., IFN production) or cytotoxic activity or proliferation. In some embodiments, the functional avidity of a TCR is expressed as the concentration needed to induce a half-maximum response (EC50). It is influenced by the affinity of the TCR for the pMHC-complex, (b) expression levels of the TCR and (c) the distribution and composition of signaling molecules, and (c) expression levels of molecules that attenuate T-cell function and TCR signaling. See e.g., Vigano S., et al. Journal of Immunology Research, vol. 2012, Article ID 153863, 14 pages, 2012 which is incorporated by reference in its entirety. Accordingly, in some embodiments, a Treg capable of suppressing a population of polyclonal T effector cells has a low affinity for the pMHC-complex. Affinity of TCR and pMHC complex can be measured using techniques such as surface plasmon resonance and expressed in terms of an equilibrium dissociation constant Kd. The lower the Kd, the higher the affinity. In some embodiments, avidity of a TCR is a measure is associated with a particular TCR expressed on a cell. In some embodiments, avidity of a TCR is a measure associated with a cell rather than a TCR expressing it because other factors, as discussed above, affect the avidity of a TCR. In some embodiments, avidity of a TCR is a measure associated with a population of cells as the heterogeneity in a population of cells can affect the avidity. In some embodiments, the exogenous TCR expressed by an engineered Treg cell has a binding affinity for an epitope on a target antigen with a binding affinity that is at least 1.5, 2.0, 2.5, 3.0, 3.5, 4.0, 4.5, 5.0, 6.0, 7.0, 8.0, 9.0, or 10.0 times lower than the binding affinity of an endogenous TCR expressed by a T effector cell in vivo.

Non-limiting examples of low avidity TCRs include, specific for IGRP and GAD65 that may be useful for treating T1D, are T1D2, T1D4, T1D5-1, T1D5-2, 4.13, GAD113, and PPI76 as described in the Examples herein.

Provided herein is a method of treating a disease or condition in a subject comprising administering to the subject a composition comprising Treg cells expressing a TCR specific to the disease or condition and capable of suppressing a polyclonal population of Teff cells.

### EXAMPLES

### Example 1—Generation of airT cells

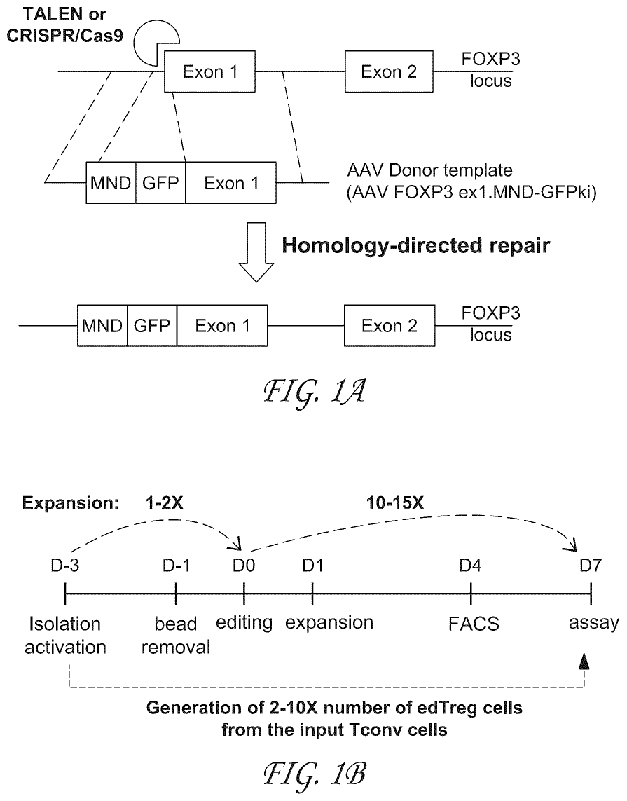

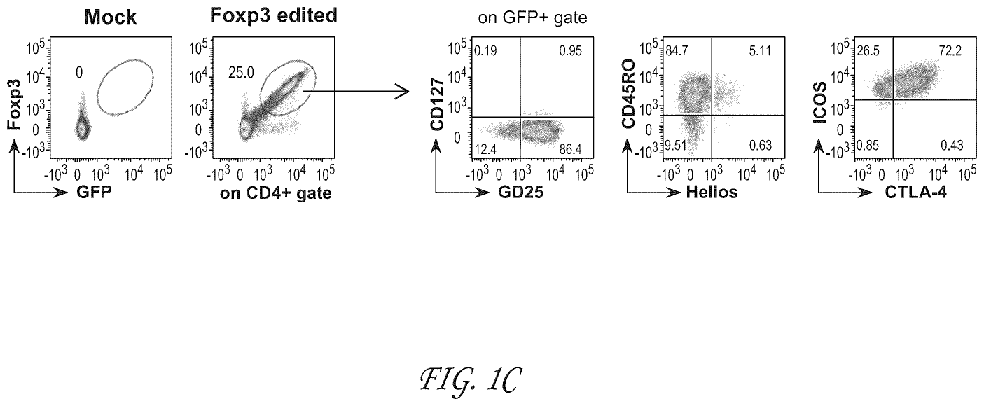

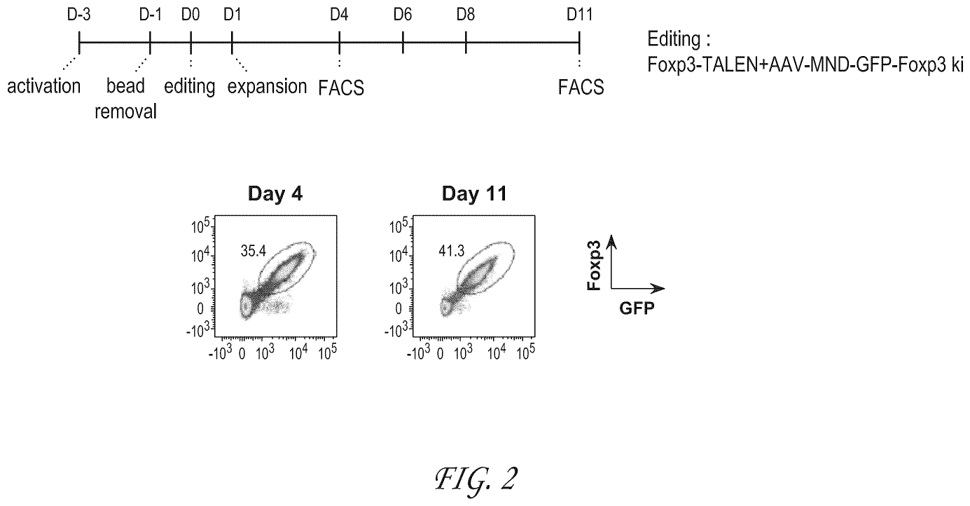

A platform was developed to generate stable engineered Treg (edTregs; airT) by converting conventional human CD4 T cells into Treg-like cells through Foxp3 gene editing (FIG. 1A, FIG. 1B, FIG. 1C, FIG. 2). This platform included the use of lentiviral TCR gene transfer to generate antigen-specific edTregs.

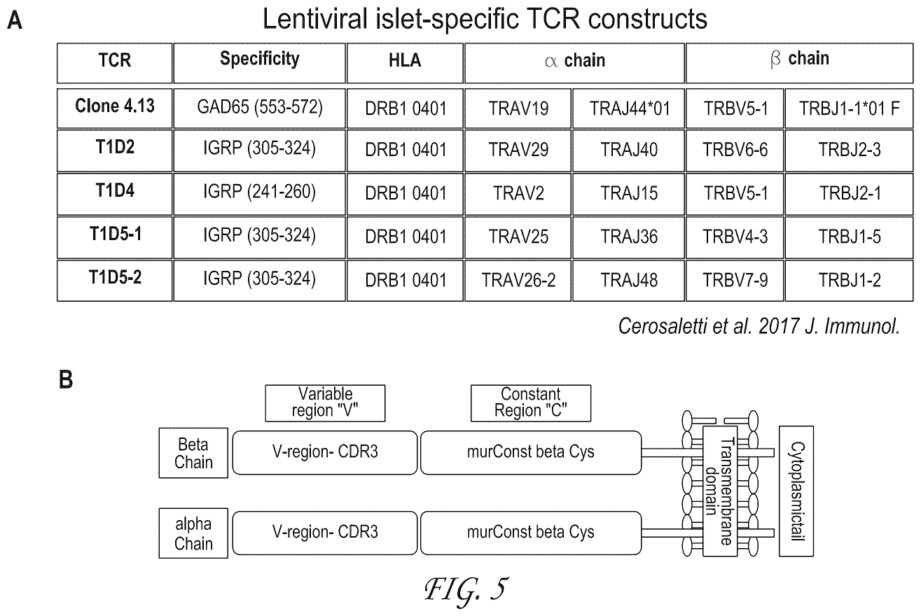

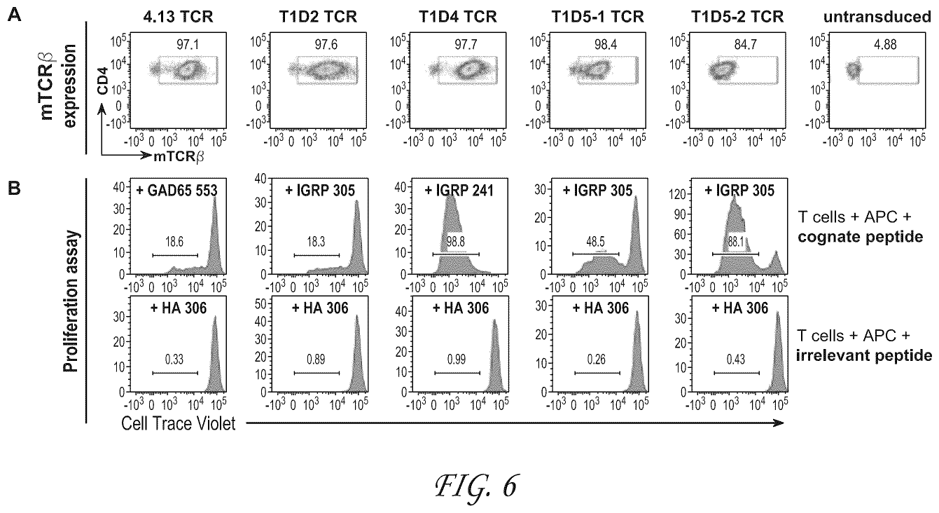

Antigen-specific T cells were identified by activating PBMC with a peptide pool, followed by assessment of CD154 expression. This method utilized single cell RNA-seq for identifying TCR clonotypes expanded in TID subjects and was used to generate full TCR sequences (Cerosaletti et al. 2017 J. Immunol. PMID: 28566371). Based on islet-specific TCR sequences identified from this study, lentiviral TCR constructs for TCR gene transfer were generated. These TCR constructs express human TCR variable regions from islet-specific TCRs and mouse TCR constant regions allowing improved pairing between the transduced human TCR chains (FIG. 5). Islet-specific TCR expression was validated by T cell proliferation assays using the TCR cognate peptides (or irrelevant peptides) with antigen presenting cells (APCs). T cells transduced with islet-specific TCRs proliferated only in response to their cognate peptides and APC (FIG. 6).

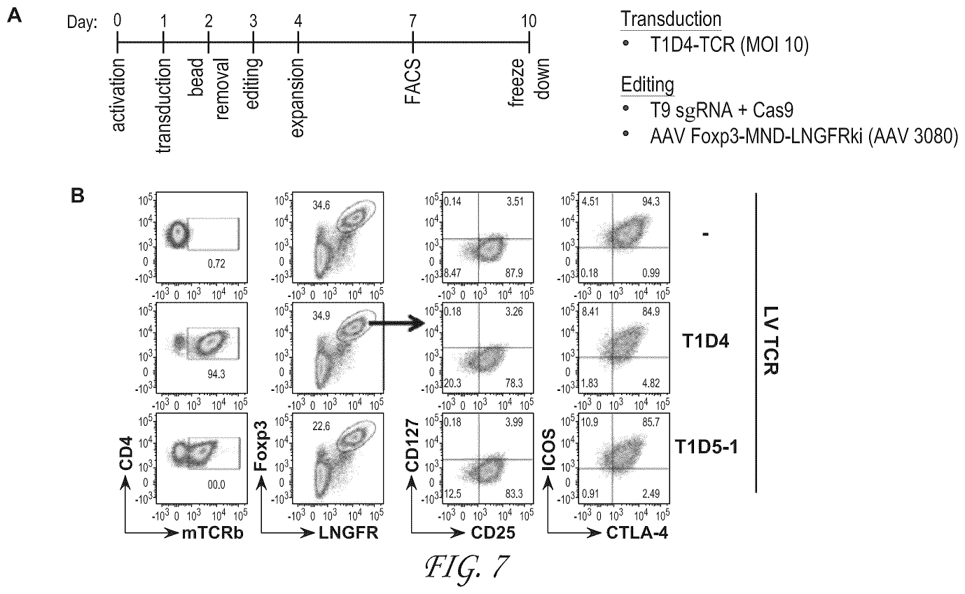

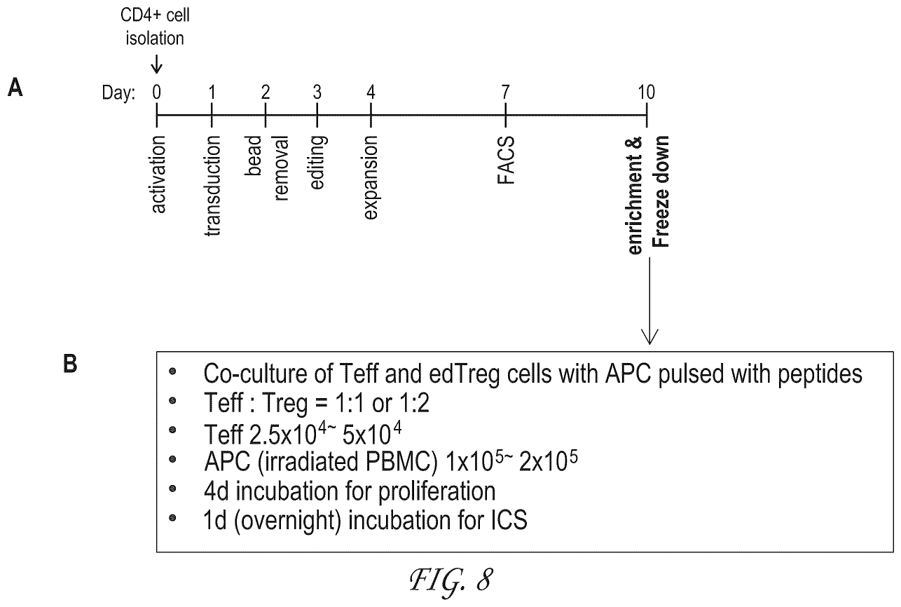

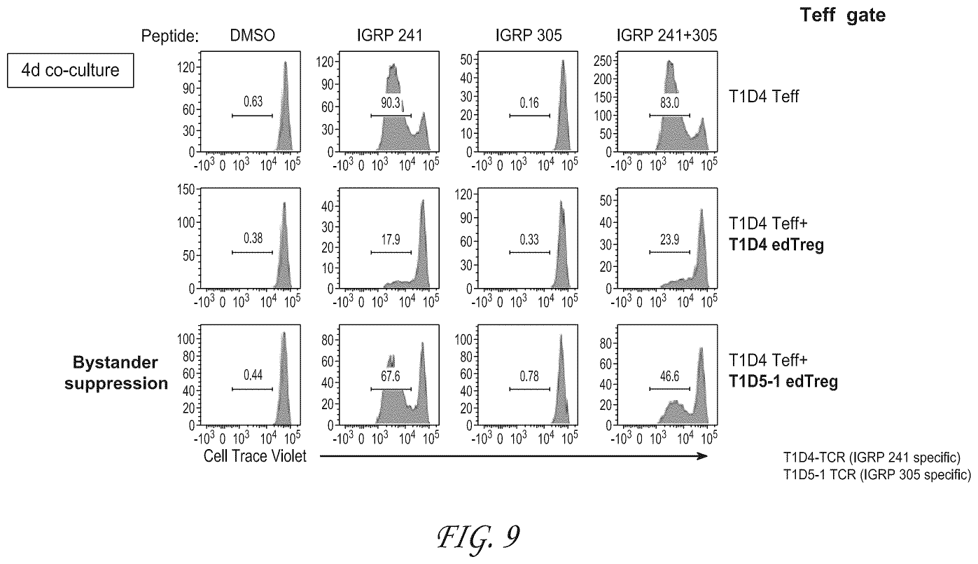

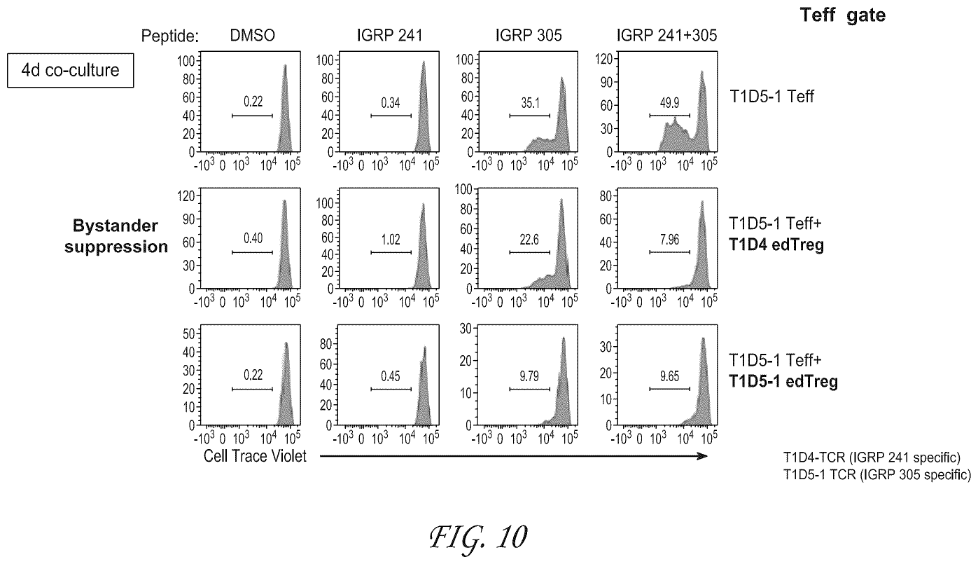

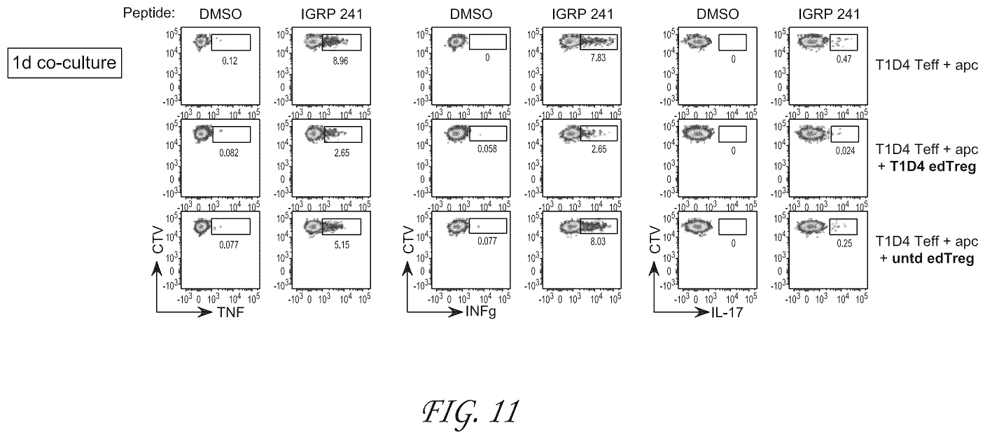

To generate antigen-specific airT cells, Foxp3 locus was edited in CD4+ T cells that had been transduced with islet-TCRs, which resulted in the successful generation of airT cells expressing islet-specific TCRs. airT expressing islet-specific TCR exhibited a Treg phenotype: CD25+, CD127−, CTLA4+, ICOS+(FIG. 7). Notably, airT expressing islet-specific TCRs show antigen-specific and bystander suppressive function by in vitro suppression assays (FIGS. 7-11). AirT cells also inhibited production of inflammatory cytokines such as TNF, IFN-g, IL-17 or IL-2 by T_{eff} cells in an airT-antigen-specific manner (FIG. 11).

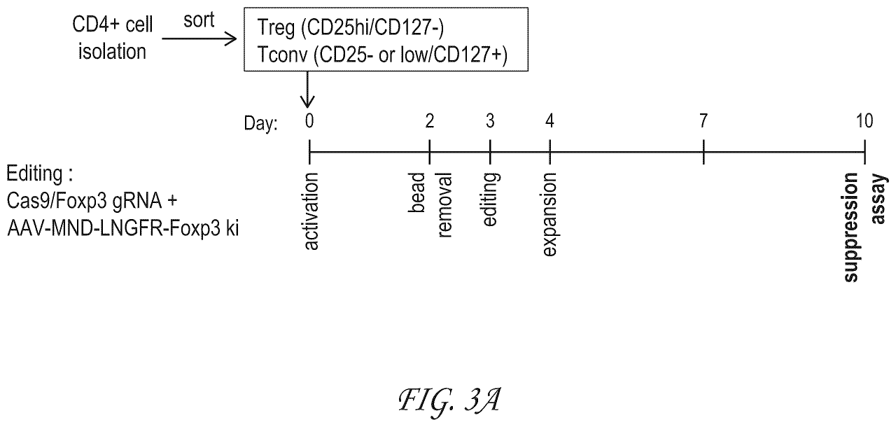

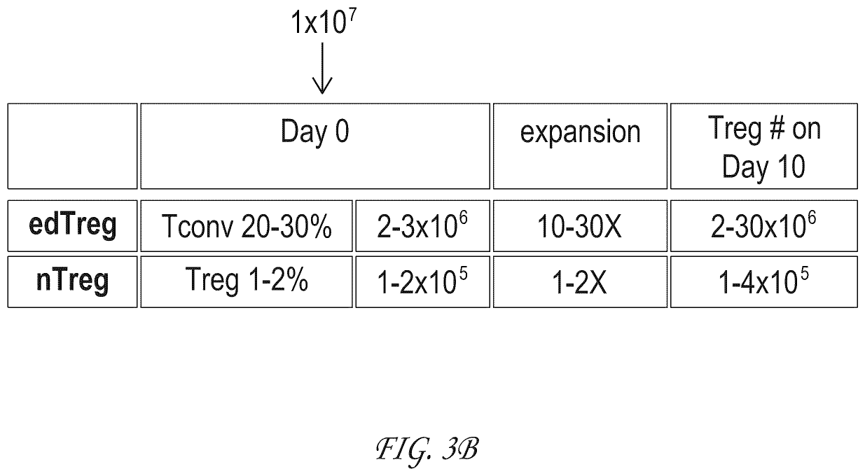

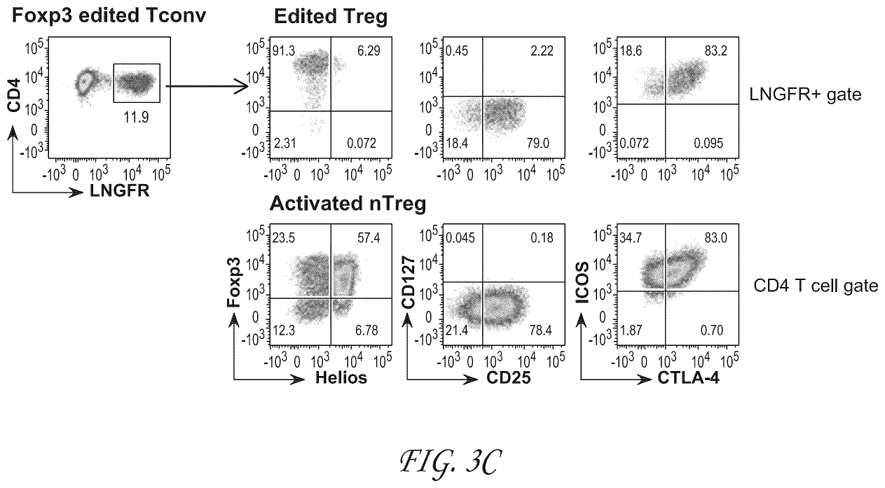

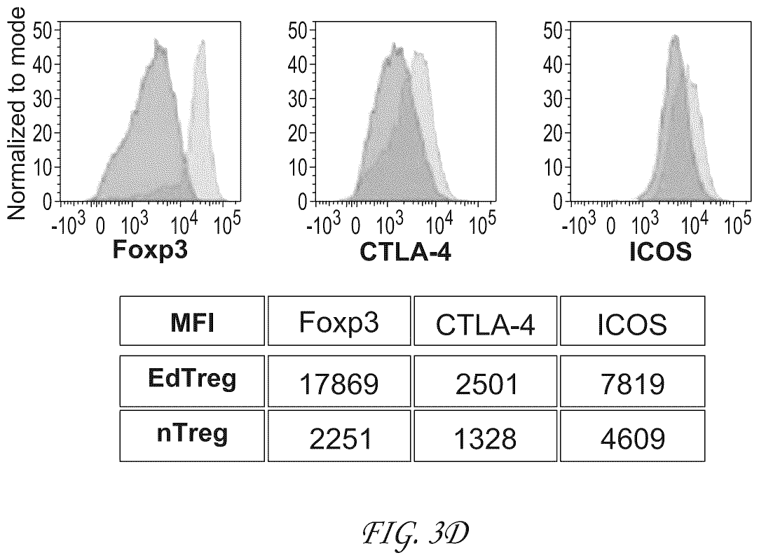

Treg phenotype, generation efficacy, and suppressive capacity of airT was investigated in comparison to expanded nTreg. airT cells could be generated up to 3 times of input number of PBMC, while the number of nTreg cells after 10 days of expansion was only 14% of the input cells. airT cells also exhibited a phenotype similar to nTreg, but showed higher expression of Foxp3, CTLA-4 and ICOS as compared to nTreg (FIG. 3).

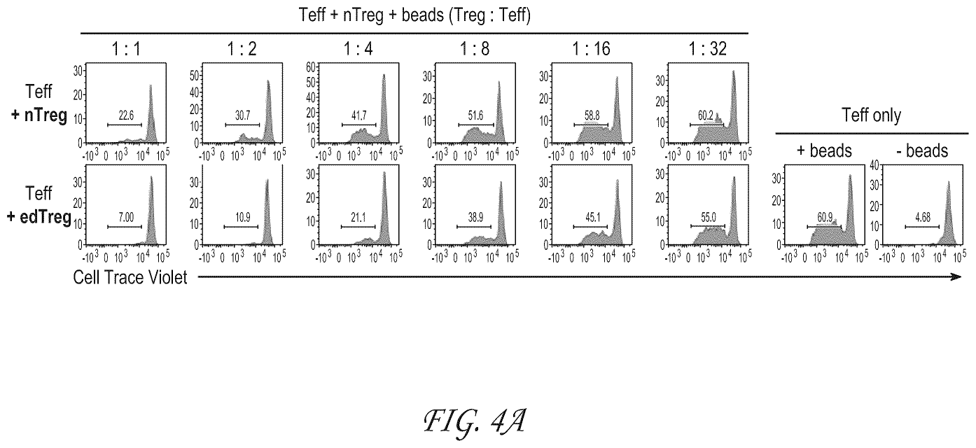

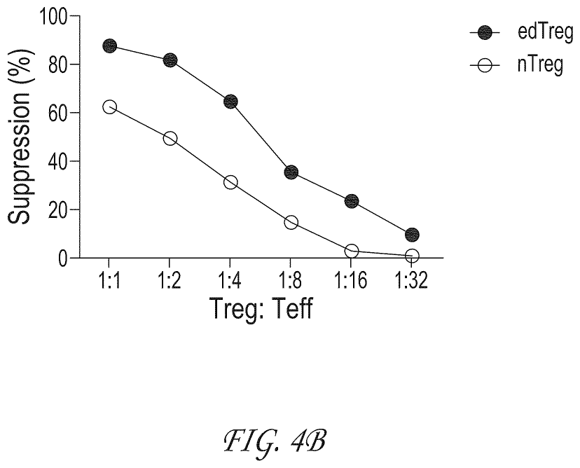

Notably, airT had similar or superior in vitro suppressive activity on effector T cell proliferation to expanded nTreg (FIG. 4).

### Example 2—Antigen-Specific Human T Cells Adopt a Treg Phenotype after FOXP3 Editing and are Immunosuppressive In Vitro

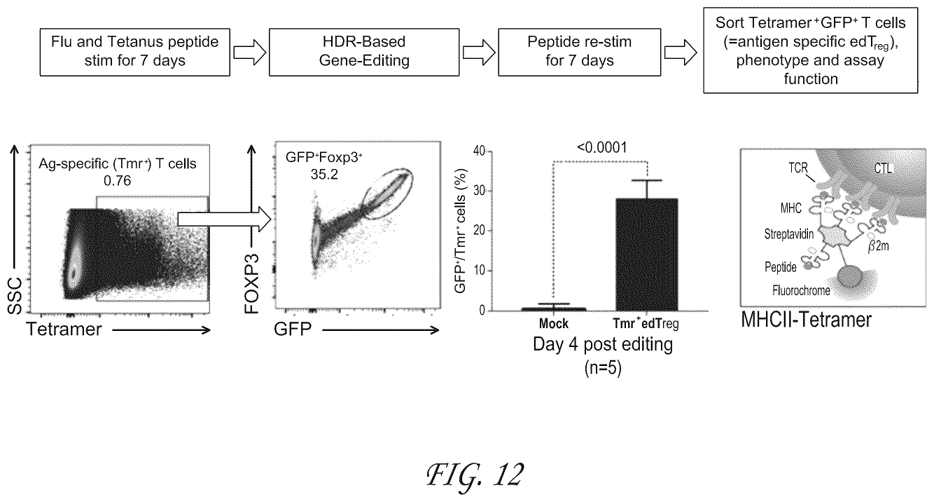

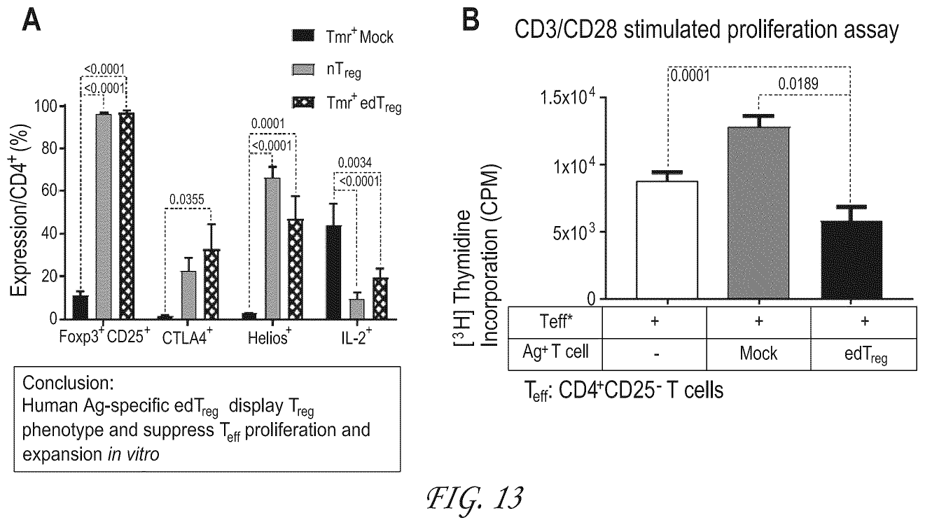

As an alternative approach to generate antigen-specific FOXP3-edited CD4+ T cells, methods were developed to isolate, edit, and expand antigen-specific effector T cells from healthy subjects or individuals with autoimmune disease. To investigate feasibility of FOXP3 editing in association with expansion of antigen-specific human T cells, CD4+ T cells from HLA DRB1*0401 human donors were expanded in the presence of influenza (flu) and tetanus antigens prior to gene editing. Following the editing procedure, the cells were further expanded in the antigen cocktail for 4 to 7 days. At this time, the average editing rate (GFP+) was 28±2.1% (FIG. 12). Antigen-specific cells were purified by FACS after labelling with a mixture of PE-conjugated flu and tetanus MHC-II tetramers. These tetramer-positive airT (Tmr+airT) recapitulated the immunophenotype of activated tTreg for canonical markers of regulatory T cells: upregulating expression of FOXP3, CD25, CTLA4, and Helios; and suppressing IL-2 production, unlike Tmr+ Mock cells analyzed in parallel (FIG. 13A). Tmr+airT were able to suppress polyclonal activated autologous CD4+ T_{eff} in vitro, unlike the Tmr+Mock cells, indicating immunosuppressive function (FIG. 13B). These results demonstrate that CD4+ T cells from human peripheral blood can be enriched for target antigen specificity by tetramer-based flow sorting and modified by gene-editing to impart tTreg-like phenotypic and suppressive properties that retain antigen specificity.

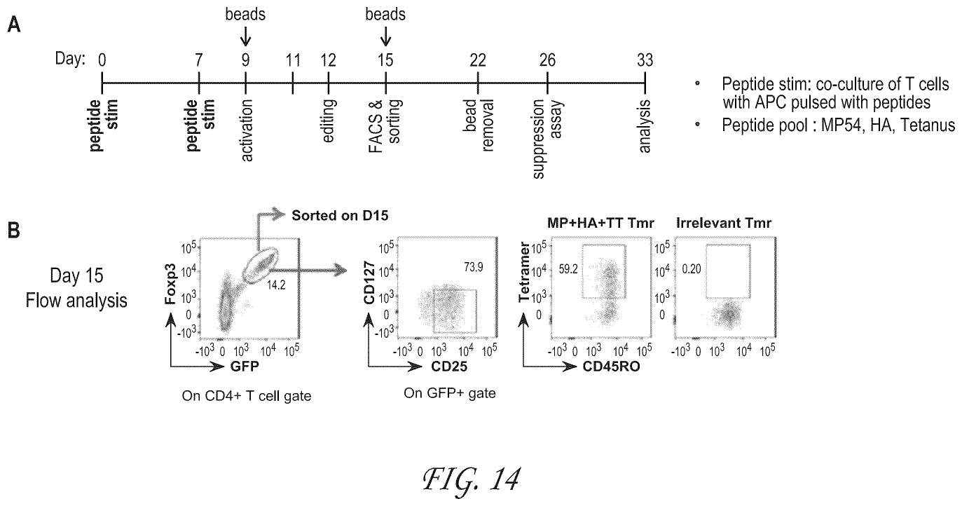

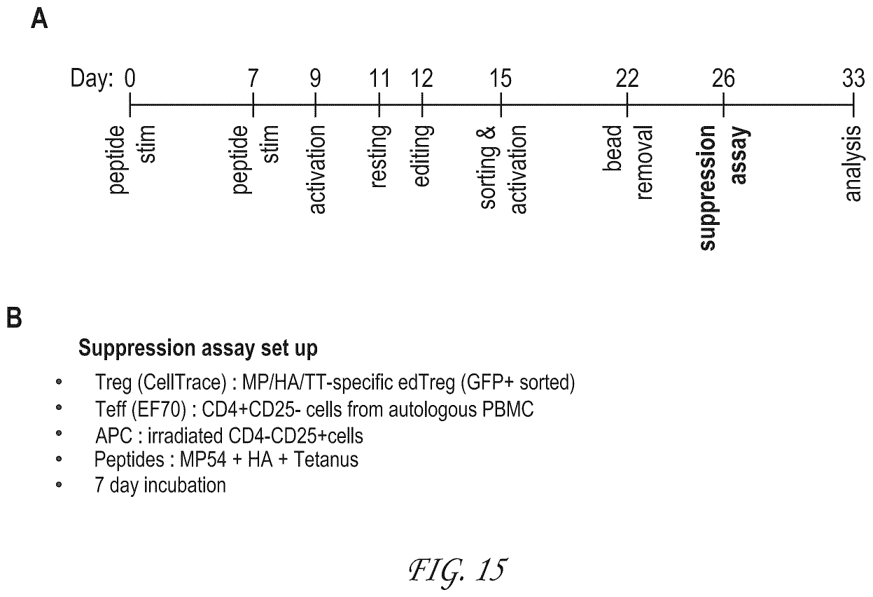

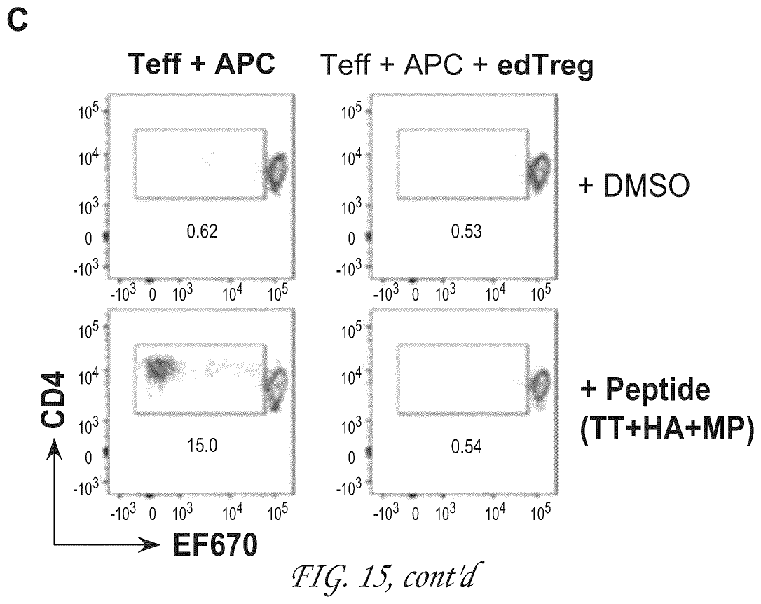

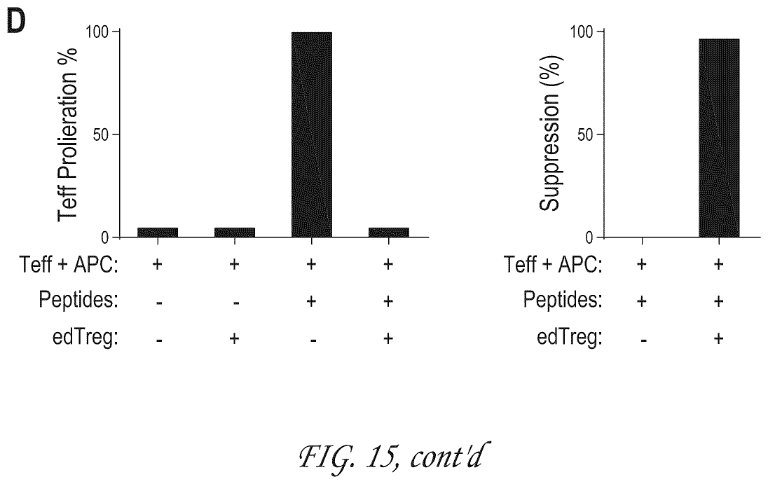

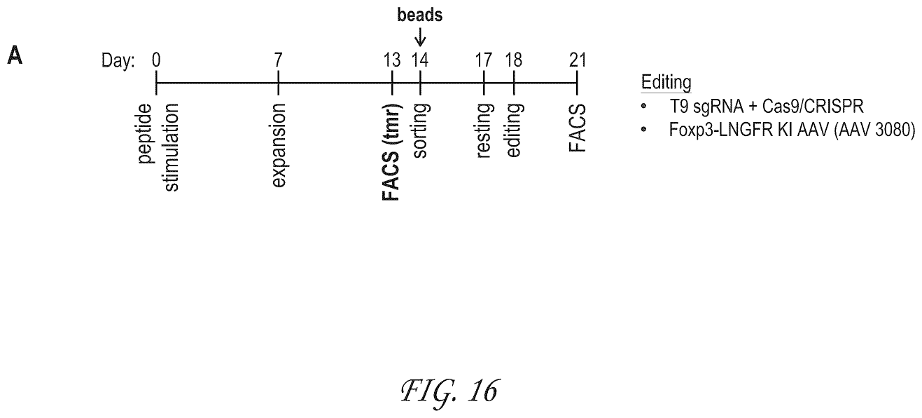

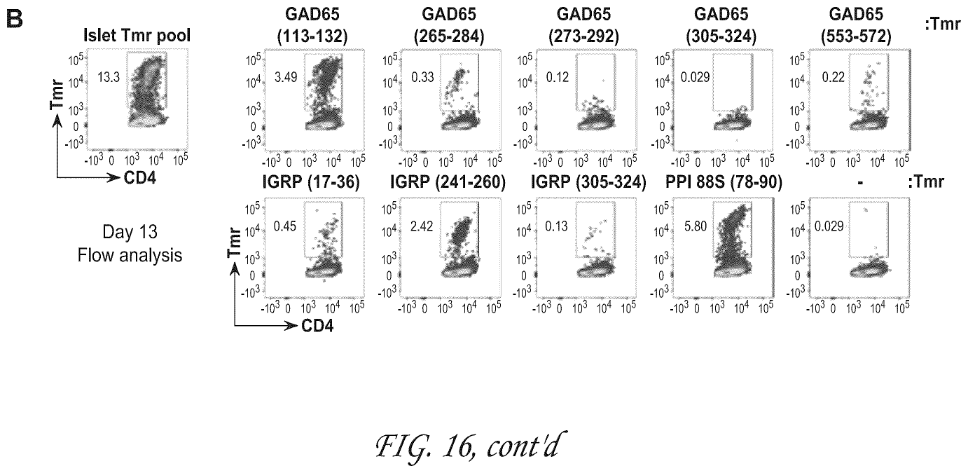

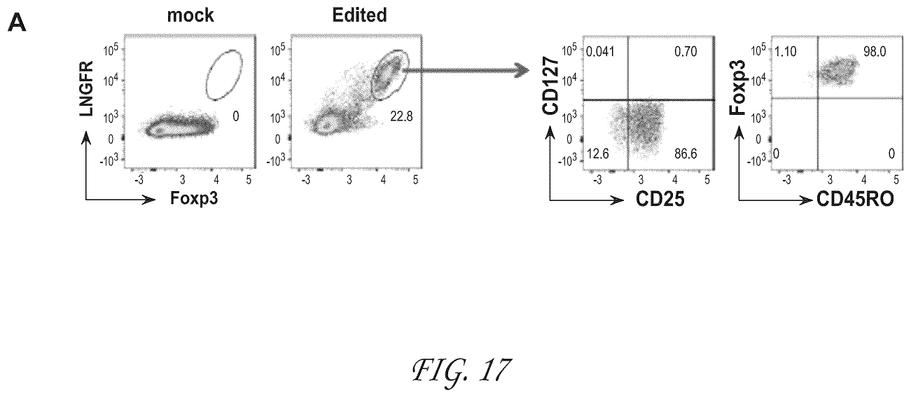

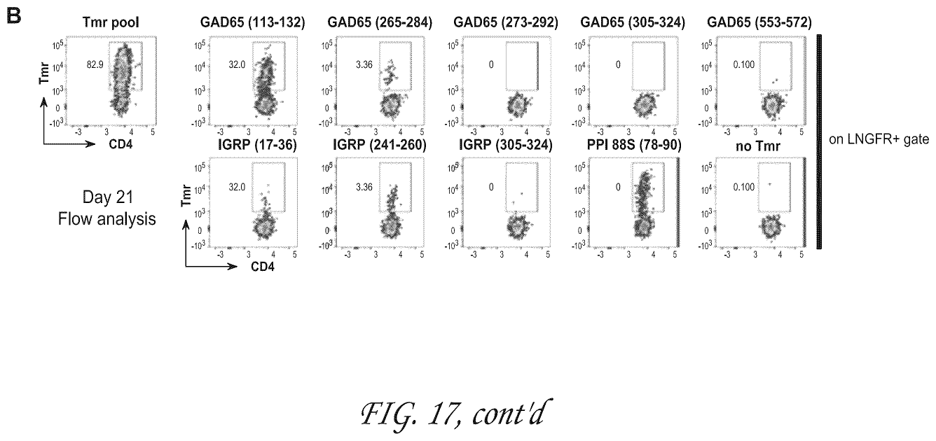

These methods were further developed to enrich for antigen-specific T cells by stimulating T cells with model antigens (MP, HA, and TT). After around 2 weeks of expansion, cells were stained with tetramers to identify antigen-specific T cells, and then the FoxP3 locus was edited (FIG. 14). Using this method, antigen-specific Tregs were generated, and these airT cells exhibited in vitro suppressive activity in antigen-specific manner (FIG. 15). In addition, islet-specific T cells were enriched by peptide stimulation method using a pool of islet-specific peptides and islet-specific T cells of multiple specificities were isolated by tetramer staining. Again, islet-specific airT cells were generated by Foxp3 gene editing in these cells (FIG. 16, FIG. 17).

### Example 3—Bi-Allelic HDR Editing for the Generation of Dual-Edited Human CD4+ T Cells

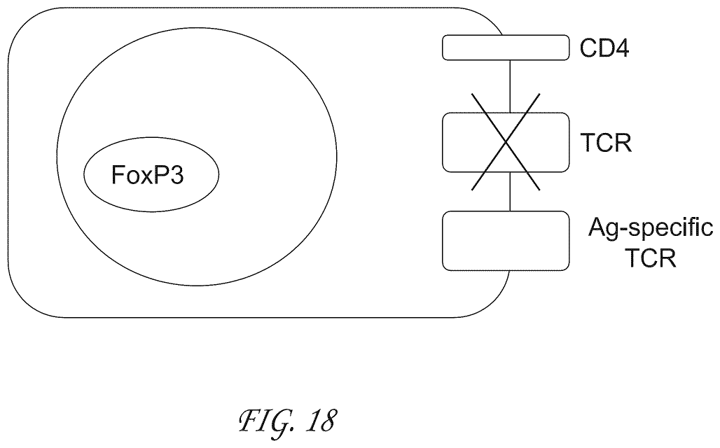

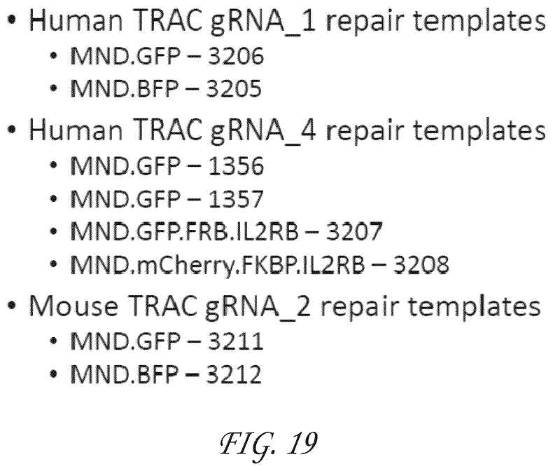

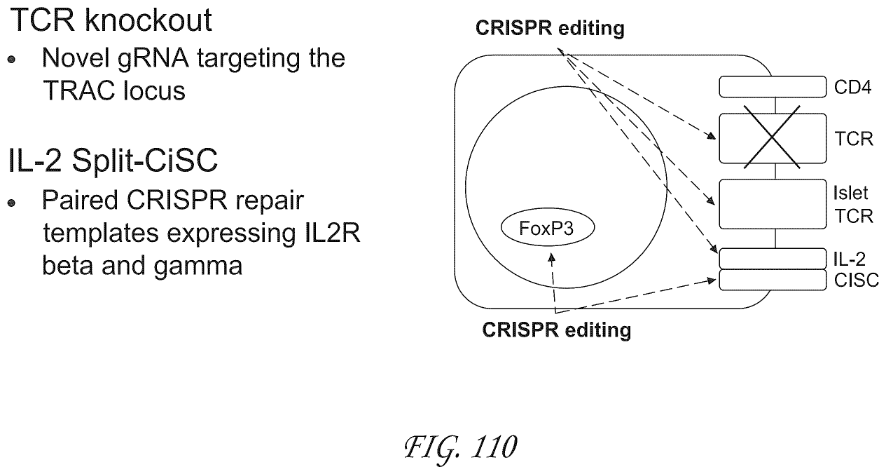

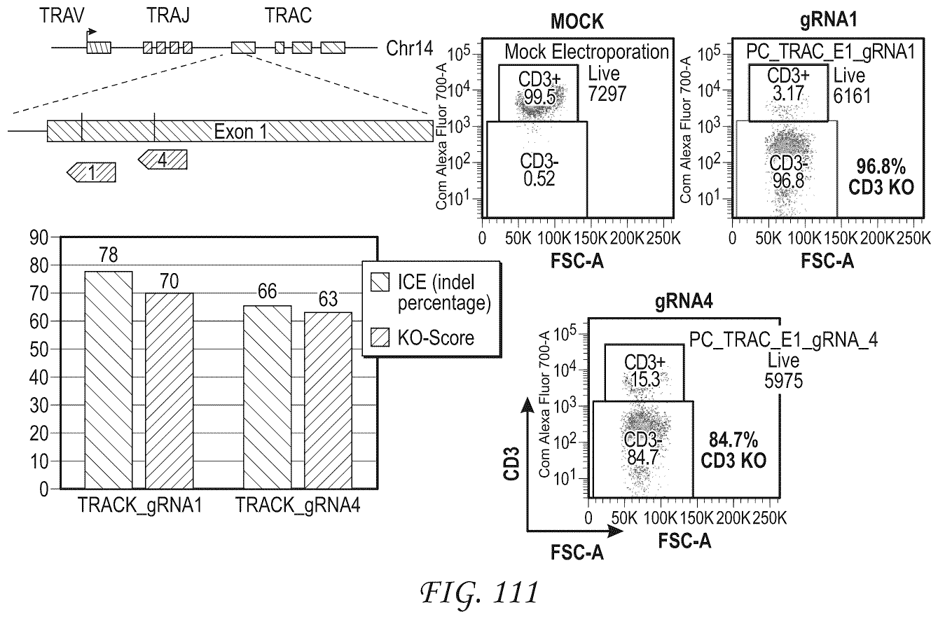

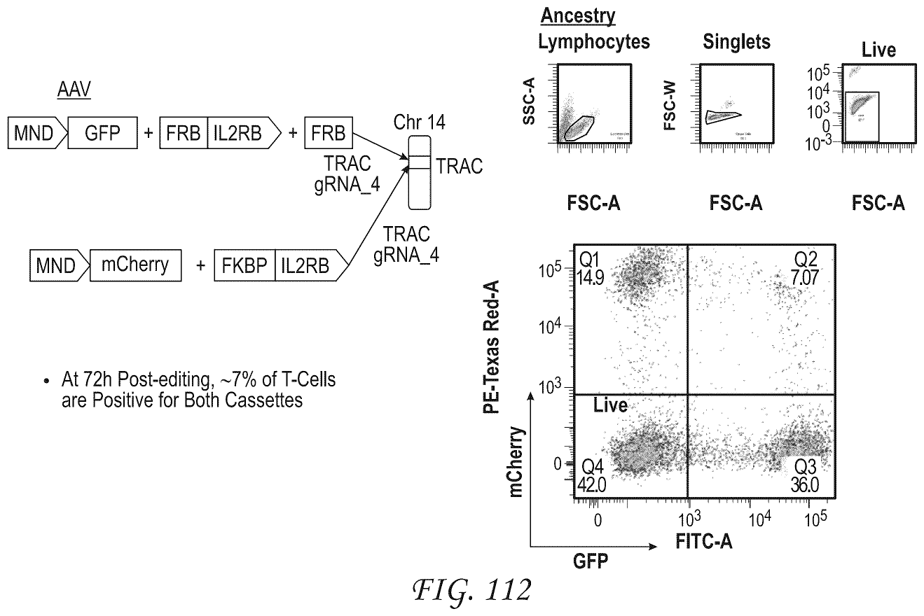

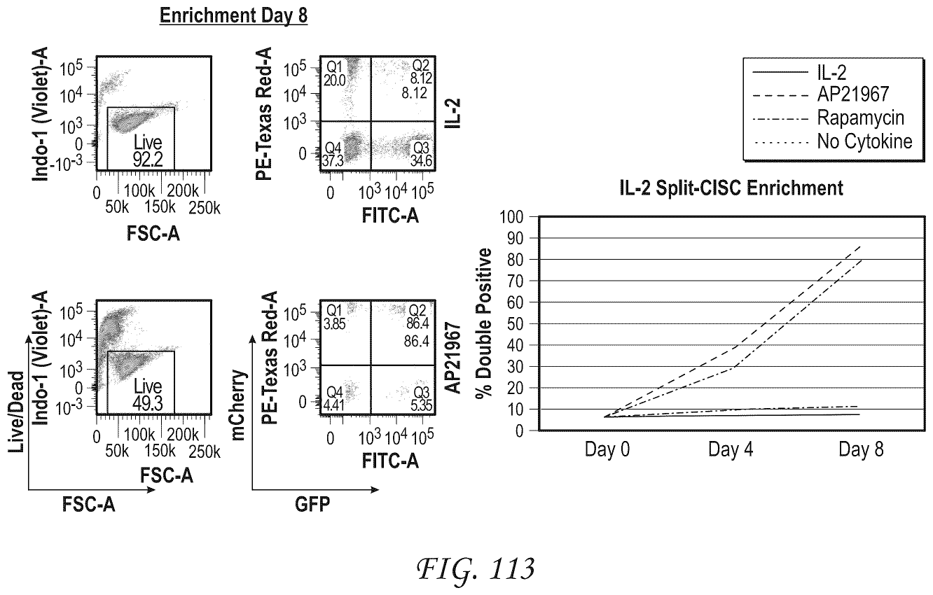

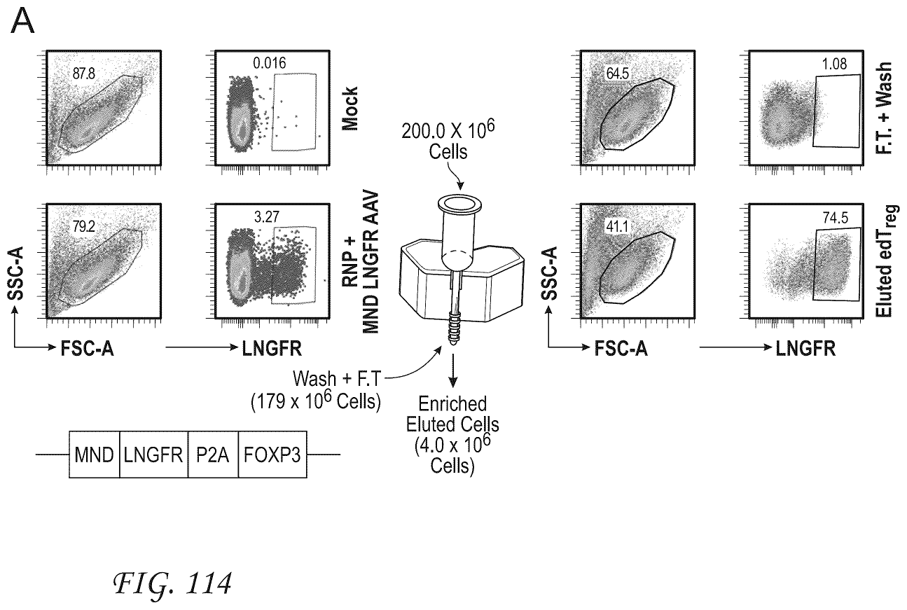

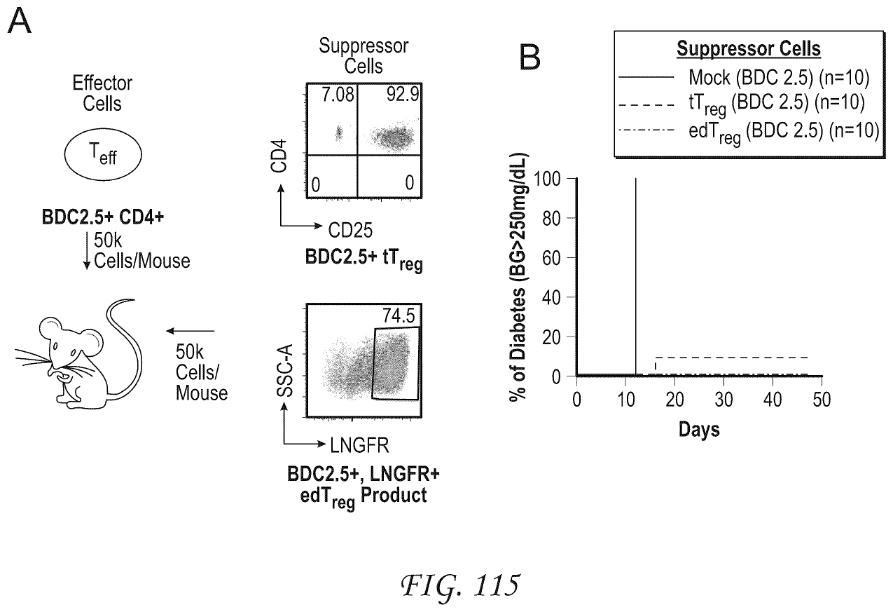

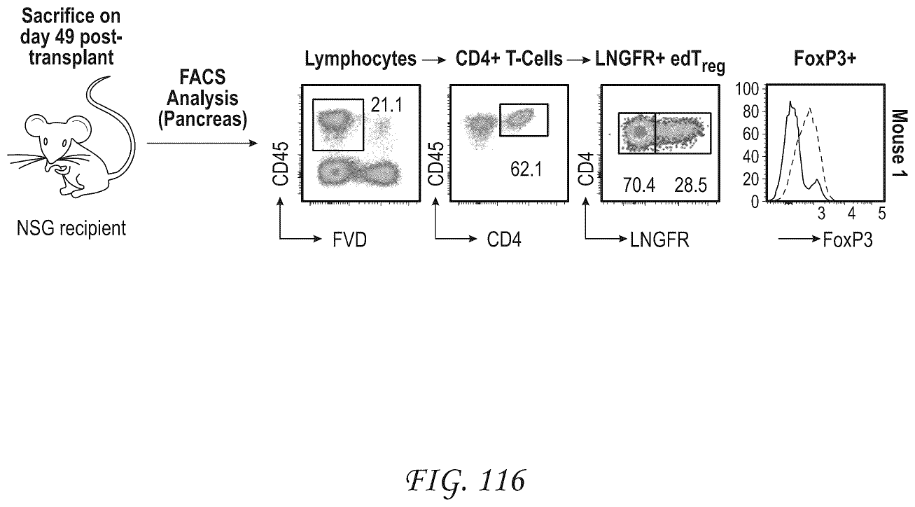

FIG. 18, FIG. 19 and FIG. 110 summarize experimental approaches used to demonstrate the ability to introduce two separate expression cassettes into the human TRAC locus (FIG. 18), as well as the constructs used in these studies (FIG. 19).

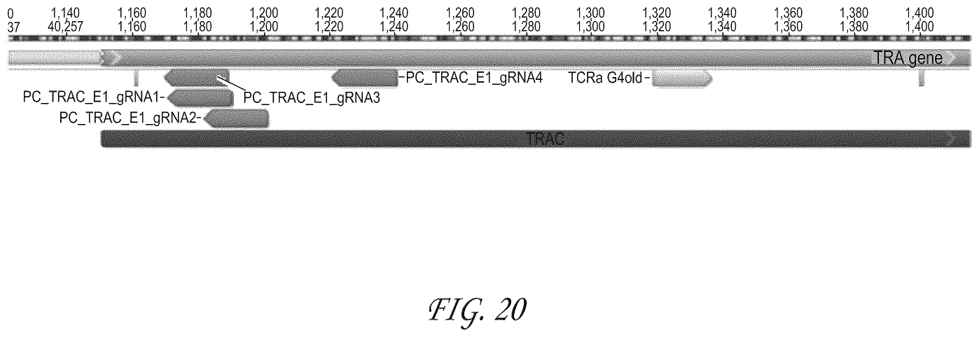

Using a CRISPR-based approach, the efficacy of four novel gRNAs targeting the first exon of human TRAC locus was tested for induction of full TCR knockout (FIG. 20). Sequences for the four gRNAs are listed in TABLE 2.

TABLE 2

| gRNA | SEQ ID NO: | Sequence |
|---|---|---|
| gRNA_1 | SEQ ID | TCTCTCAGCTGGTACACGGC |
| (human TRAC | NO: 1405 | |
| editing) | | |
| gRNA_2 | SEQ ID | TGGATTTAGAGTCTCTCAGC |
| (human TRAC | NO: 1428 | |
| editing) | | |
| gRNA_3 | SEQ ID | CTCTCAGCTGGTACACGGCA |
| (human TRAC | NO:1429 | |
| editing) | | |
| gRNA_4 | SEQ ID | GAGAATCAAAATCGGTGAAT |
| (human TRAC | NO: 1406 | |
| editing) | | |

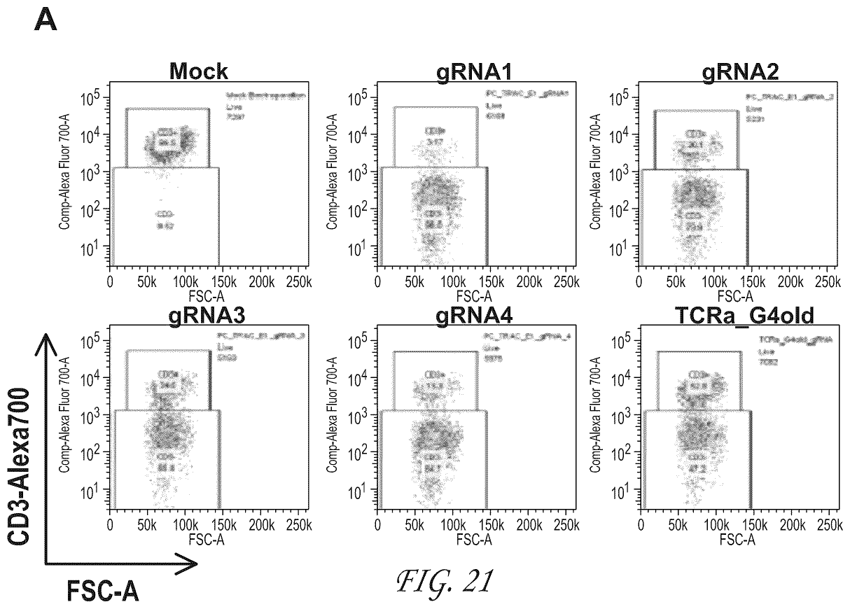

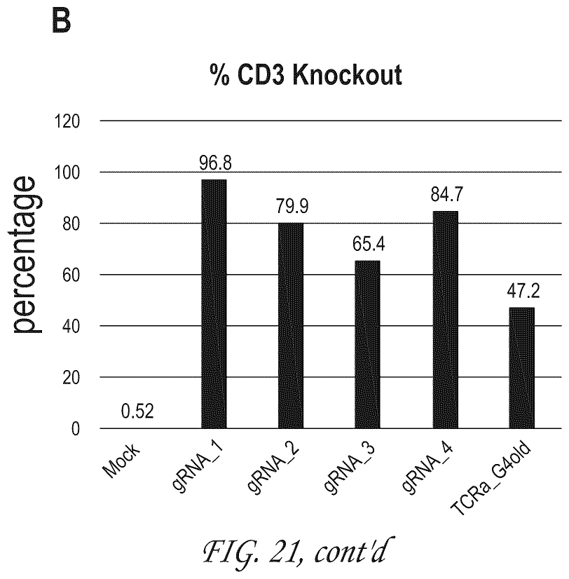

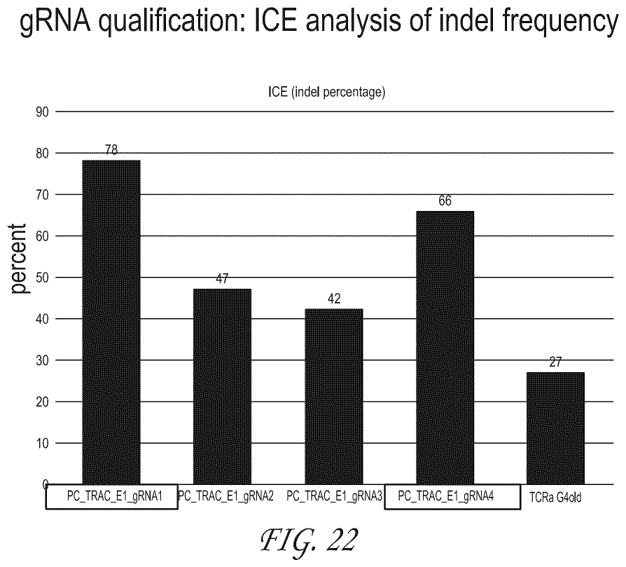

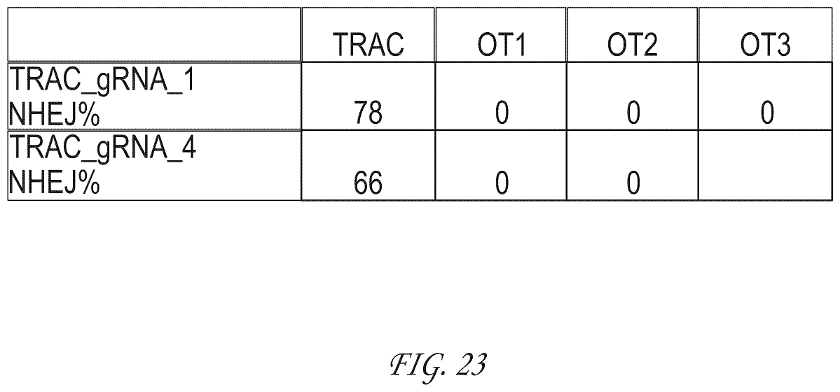

CD3 expression was evaluated using flow cytometry 48 hr after RNP delivery and demonstrated 96.8% and 84.7% CD3 knockout using gRNA_1 and gRNA_4, respectively (FIG. 21). On-target site-specific activity was measured by ICE (Inference of CRISPR Edits) and confirmed specific indel induction for gRNA_1 and gRNA_4 in TRAC relative to predicted off-target sites (FIG. 22). Next, to test the specificity of the novel guides, the top three off-target sites for each gRNA (as predicted based on bioinformatics looking at the most similar sequences in the human genome) were assessed. These were then directly analyzed in human T cells via amplification of the off-target site from T cells transfected with the nuclease. The amplicons were sequenced and analyzed by ICE program. The level of cleavage activity observed for the candidate off-target sites was 0% cleavage. In contrast, on-target site activity in the same assay was 78% for gRNA_1 and 66% for gRNA_4 at the target TRAC site (FIG. 23). This illustrates that these novel donor templates are highly-specific for the TRAC locus.

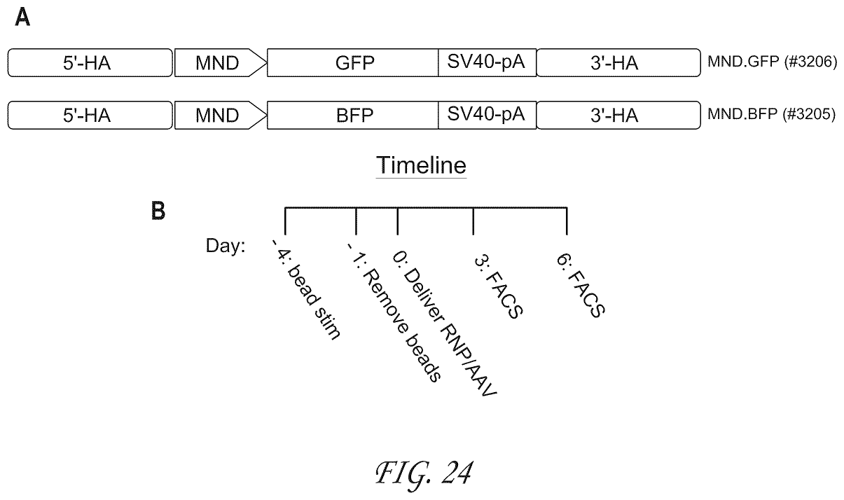

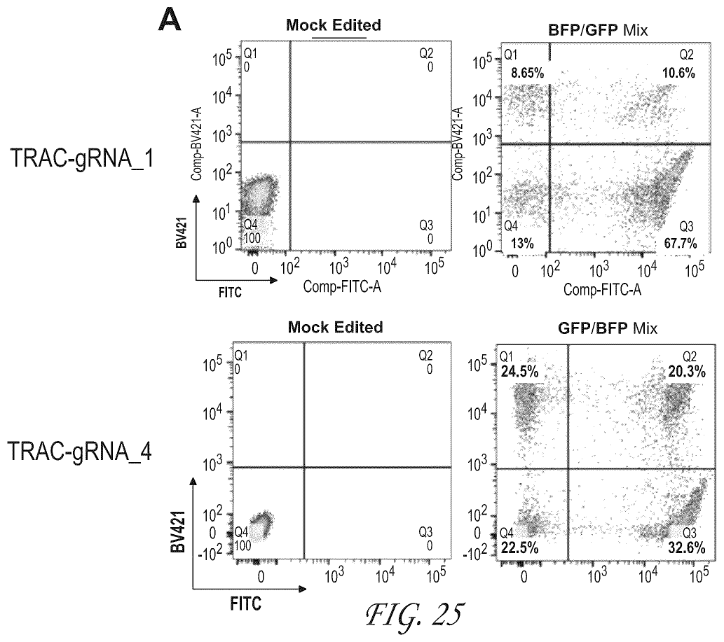

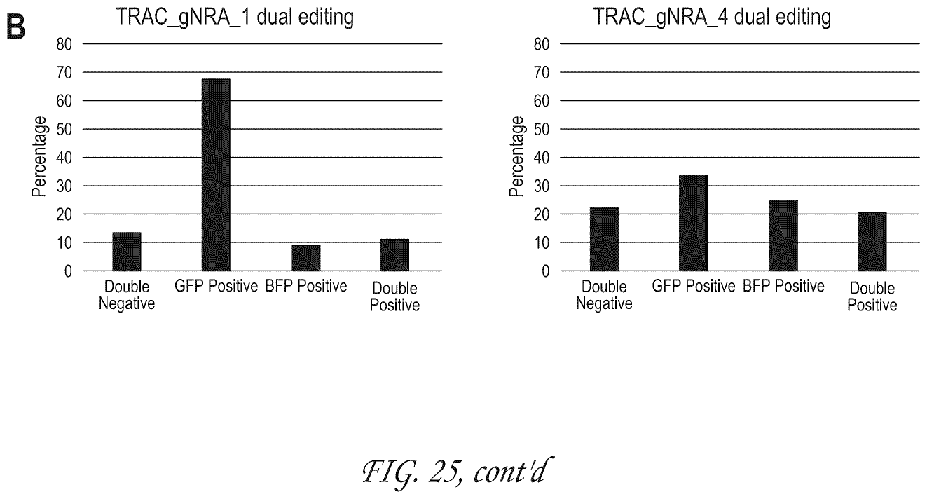

Next, the ability to dual edit human CD4+ T cells was tested using constructs that allow easy tracking of successfully edited cells. MND-GFP and MND-BFP cassettes were generated, flanked by identical 300 bp homology arms matched to TRAC gRNA_1 or gRNA_4 (FIG. 24A), and were used to test the ability to generate biallelic TRAC edited T cells with stable expression of both GFP and BFP. The timeline for cell expansion, editing and analysis is shown in FIG. 24B and the resulting FACS analysis demonstrated 20.3% and 10.6% BFP/GFP double-positive cells using gRNA_1 and gRNA_4, respectively, confirming successful integration of both repair cassettes after induction of a single double strand break (FIG. 25).

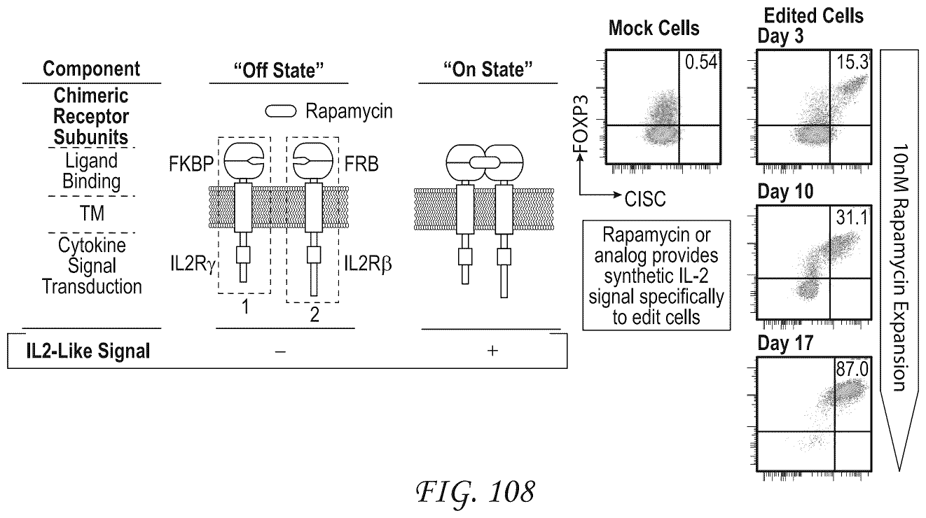

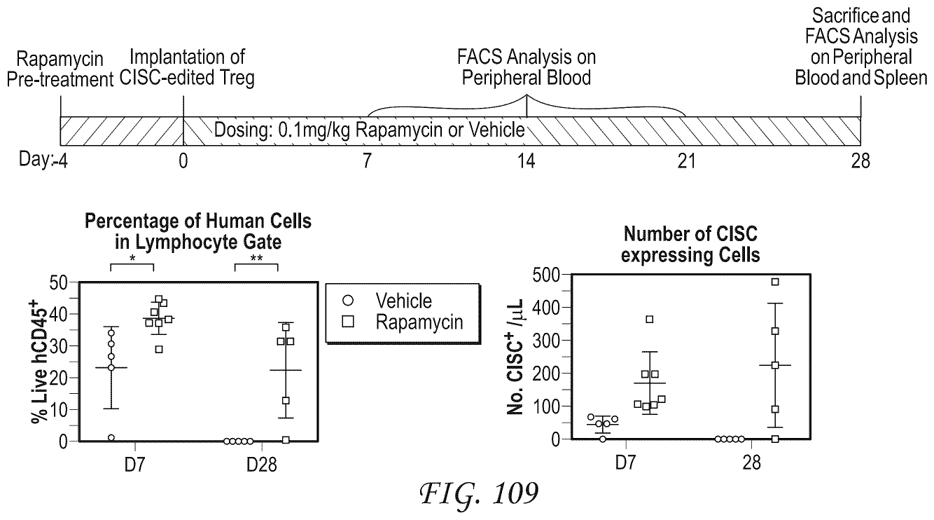

In order to obtain sufficient numbers of cells for therapeutic use, it may be useful, in some contexts, to selectively expand engineered cells in vitro. To do this in the context of dual-edited cells, split IL-2 CISC HDR knock-in constructs were generated for enrichment and selection. The method of using IL-2 CISC components has been described for the enrichment of edited CD4+ T-cells in the presence of rapamycin or a heterodimerizing rapamycin homolog, AP21967 (rapalog). See e.g., FIG. 108. In such methods, FRB-IL2RB and FKBP-IL2RG components were contained in the same cassette to select for single integration events.

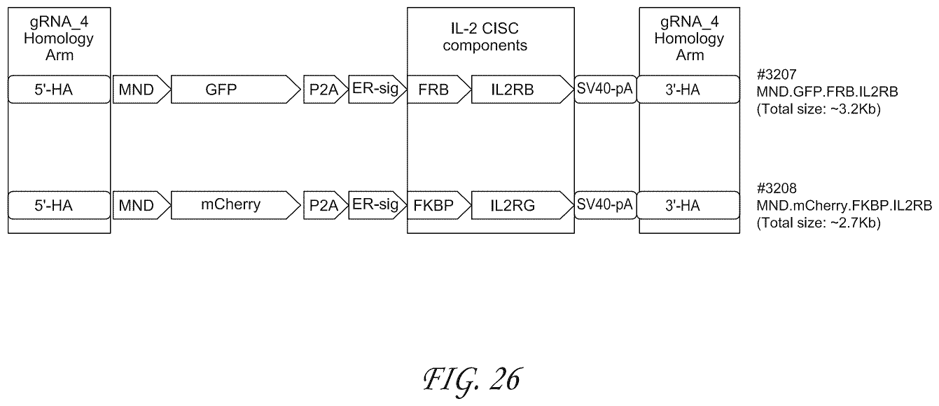

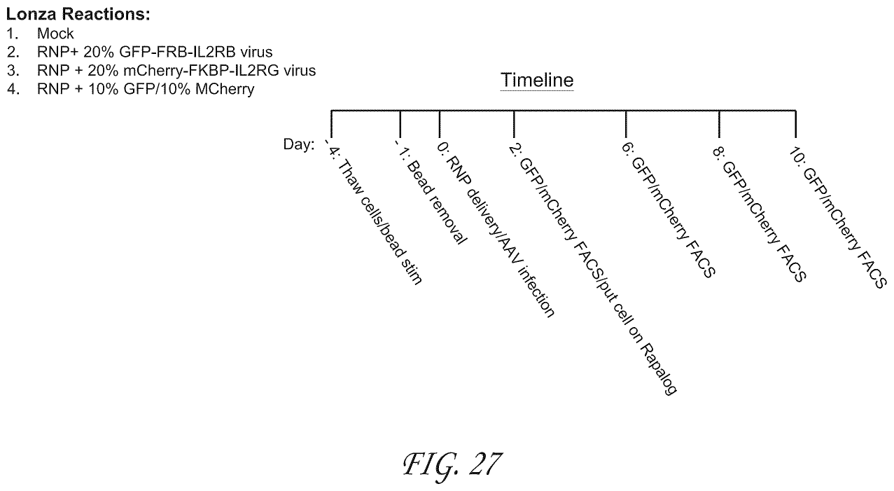

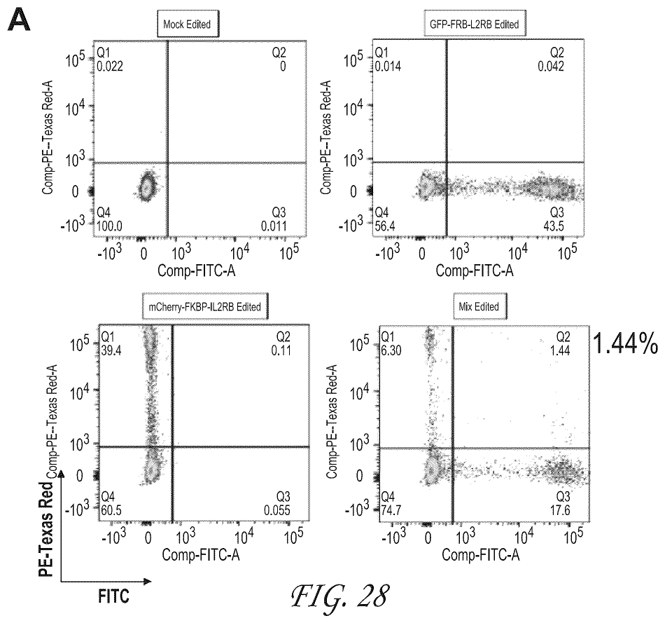

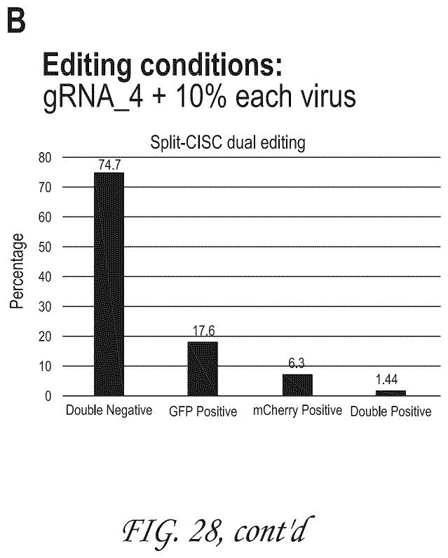

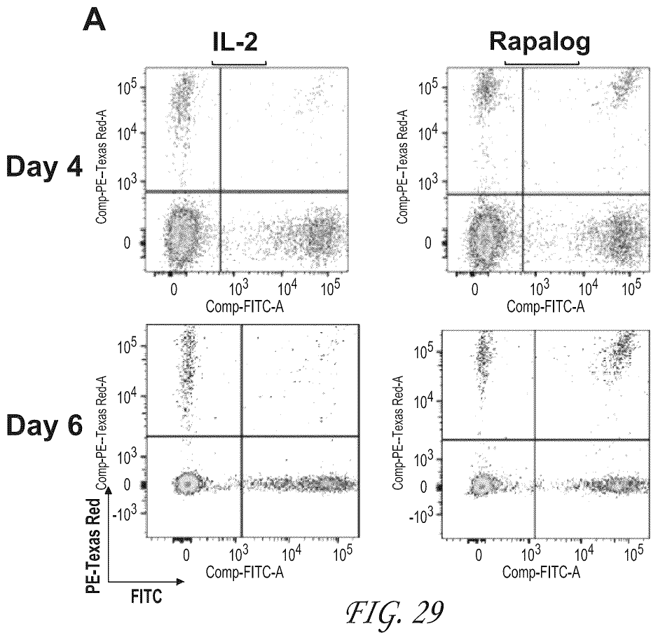

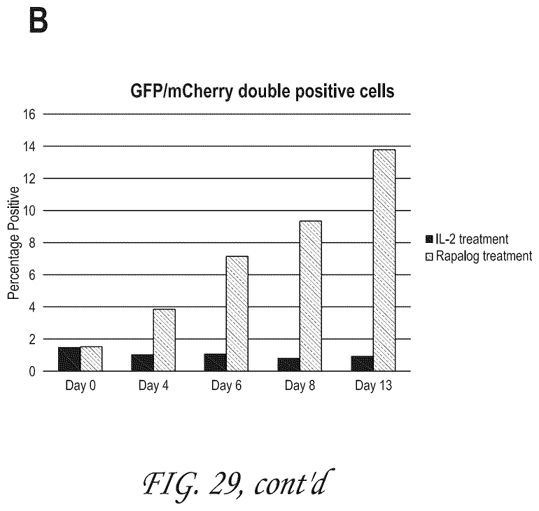

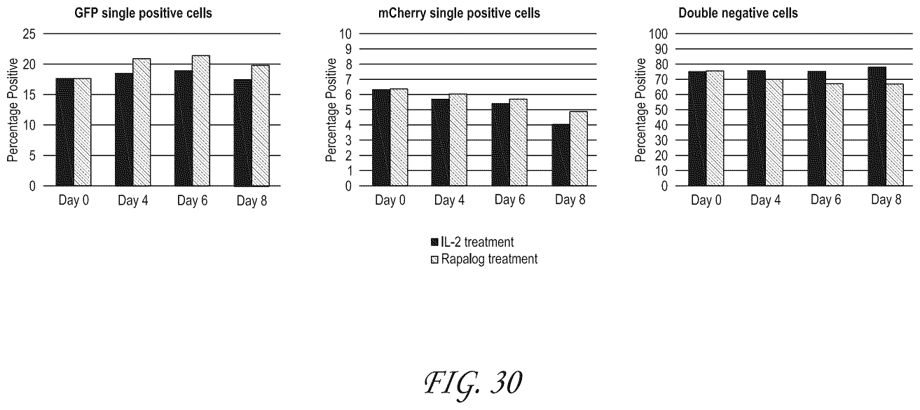

For these studies, FRB-IL2RB and FKBP-IL2RG components were split into two separate cassettes, one containing GFP and the other containing mCherry, to allow for selection of two independent integrations. Constructs are shown in FIG. 26 and the timeline and editing conditions for this experiment are shown in FIG. 27. Although the initial dual editing rate with these constructs was 1.44% double positive GFP/mCherry cells, potentially due to the increased HDR template size, FIG. 28, dual edited cells could be significantly enriched-for using rapalog. In the presence of 100 nM rapalog treatment, GFP/mCherry double positive cells increased from 1.4% to 9% over 8 days (FIG. 29). Importantly, GFP single-positive, mCherry single-positive and double-negative cells percentages remained the same in the presence of rapalog, suggesting that expansion only takes place when a functional IL-2 CISC protein is present through dual expression of FRB-IL2RB and FKBP-IL2RG. As expected, no expansion was observed in the presence of IL-2 (FIG. 30).

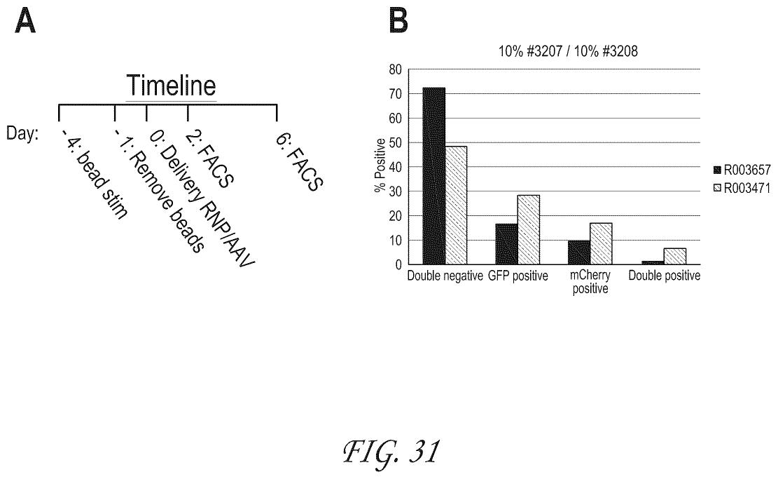

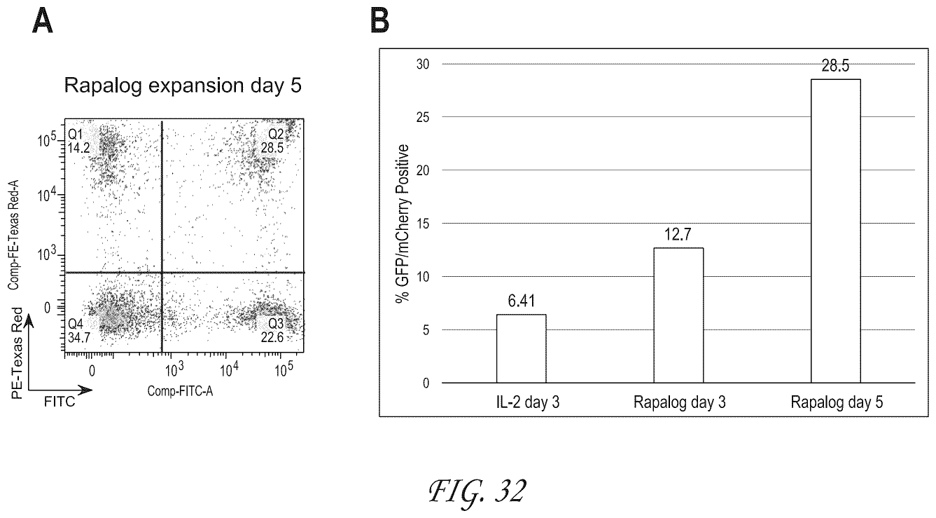

The reproducibility between experiments and variance between donors was tested (FIG. 31). Cells from the same donor from the previous experiment (shown in FIG. 29) were edited and compared with cells from an additional male, Caucasian donor of similar age. The percent dual editing of R003657 donor was 1.1%, which is similar to what was observed previously (FIG. 29). Bi-allelic editing of the second donor, R003471, was 6.4%. Overall, the editing rate changed between donors, but the ratio between GFP-positive, mCherry-positive and double-positive cells remained similar, suggesting variability may be based on how well the donor can be edited. Importantly, dual-edited cells from both donors were successfully enriched-for in the presence of Rapalog, yielding 13.8% and 28.5% GFP/mCherry double-positive cells for donors R003657 and R003471, respectively (FIG. 32).

The results of these studies suggested that incorporation of the split IL-2 CISC in dual HDR editing provides a means of efficient selection and enrichment of dual-edited cells and could provide a method to obtain edited cell numbers necessary for therapeutic use.

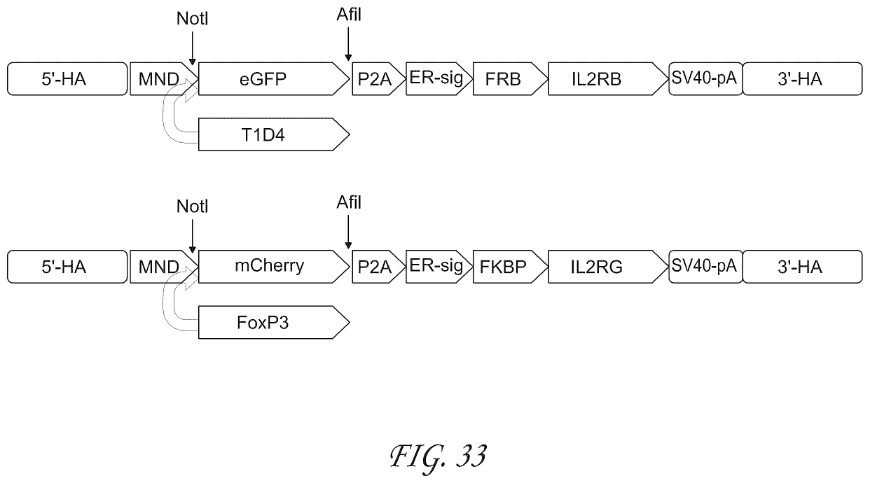

In view of the successful bi-allelic editing using MND-eGFP-FRB-IL2RB and MND-mCherry-FKBP-IL2RG cassettes, and enrichment of dual-edited cells using Rapalog, constructs were generated to introduce FoxP3 and a pancreatic islet antigen-specific TCR (T1D4) in combination with the IL-2 CISC components to generate antigen-specific Foxp3+ airT cells (FIG. 33).

### Example 4—Bi-Allelic Targeting for the Generation of Dual Edited Murine CD4+ T-Cells

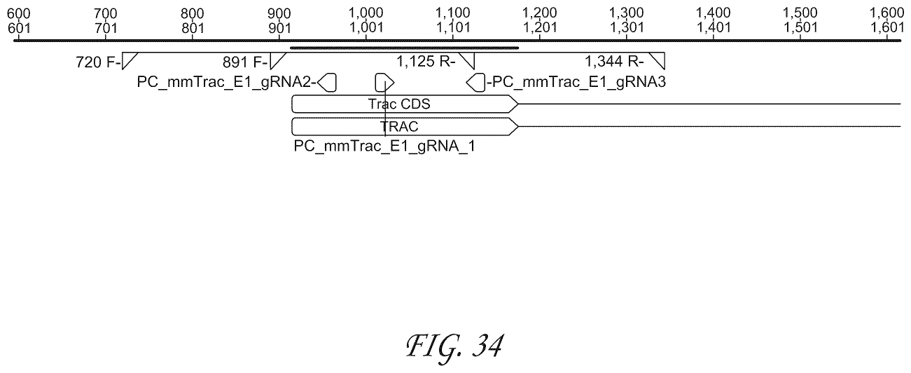

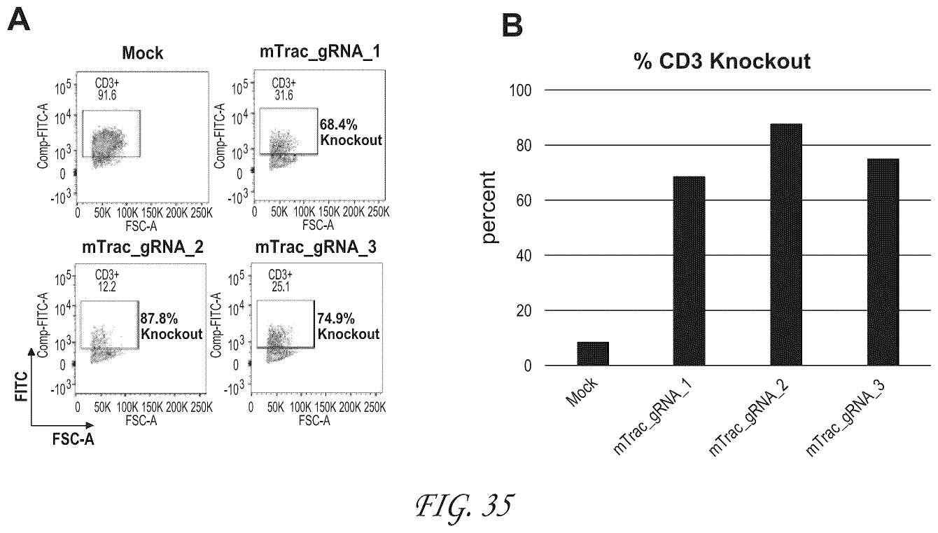

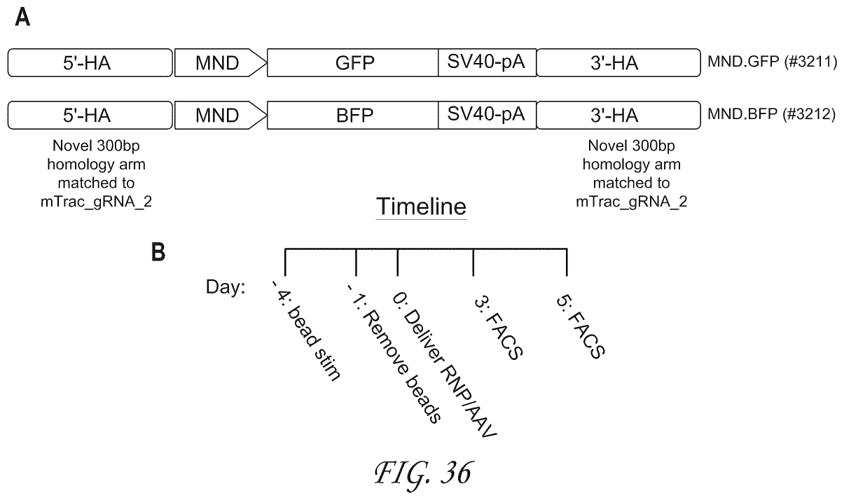

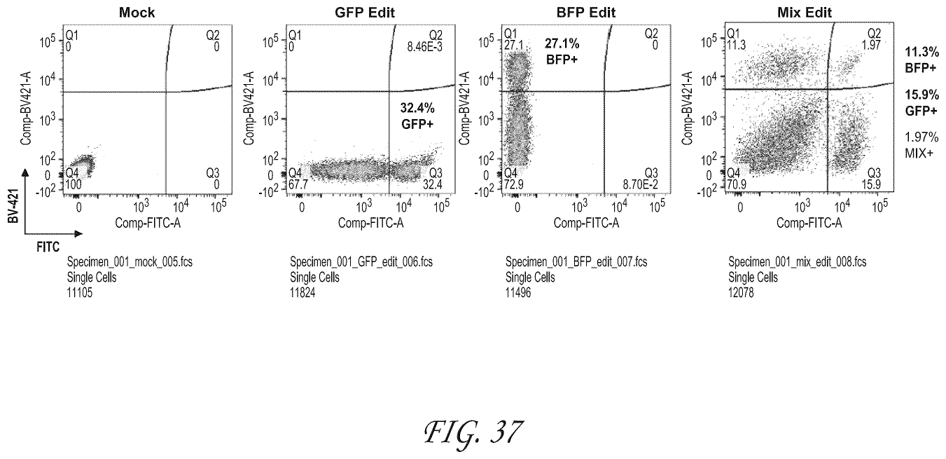

In order to perform studies to assess the efficacy of Ag-specific FoxP3 airT in animal models of diabetes or other autoimmune conditions, analogous tools for editing into the murine Trac locus were generated. Three novel gRNA target sequences within the first exon of murine Trac locus were selected and tested for CD3 knockout in mouse (C57/B6) CD4+ primary T cells (FIG. 34). FIG. 35 shows that mTrac_gRNA_2 resulted in the best knock-out of 87.8%, as measured by flow analysis of mCD3 expression following 2-days post transfection. As with the human construct, MND-GFP and MND-BFP constructs were generated to enable convenient tracking of edited cells. The construct and timeline for this experiment is shown in FIG. 36. As was the case with dual-editing human cells at the TRAC locus, the dual editing efficiency in murine cells was relatively low (1.97%) (FIG. 37).

### Example 5: airT Function in an Antigen-Specific Murine Model of Multiple Sclerosis

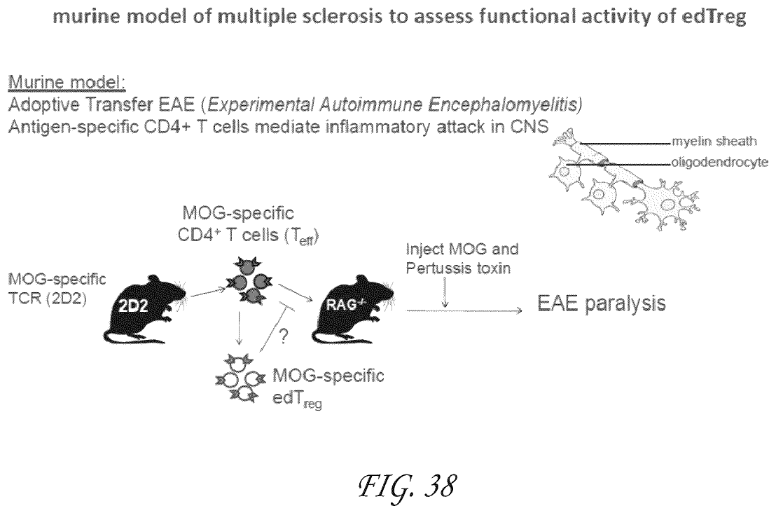

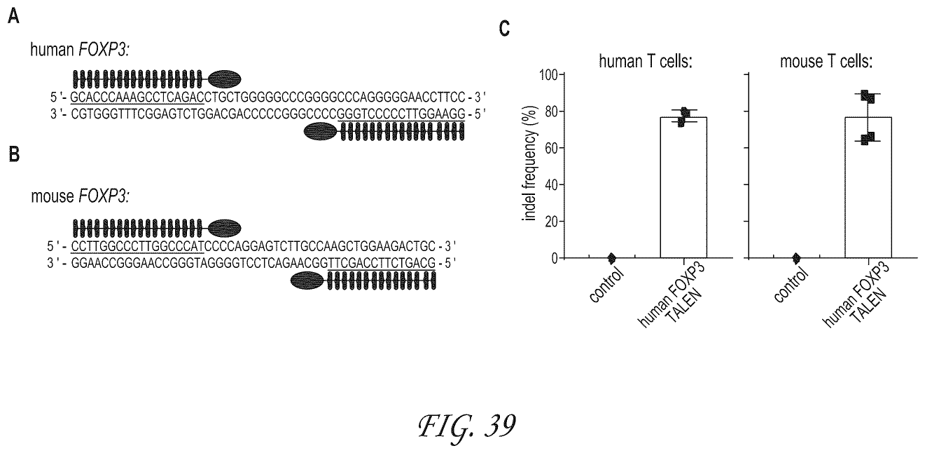

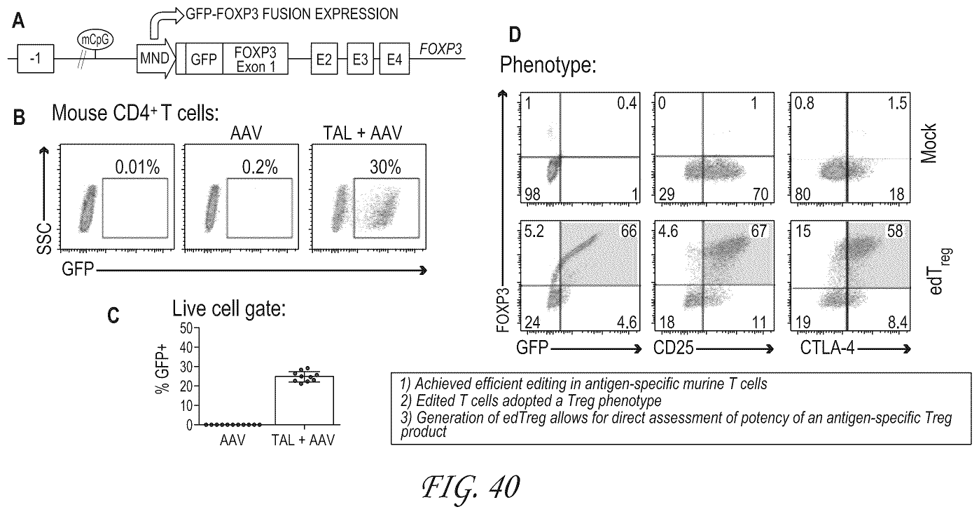

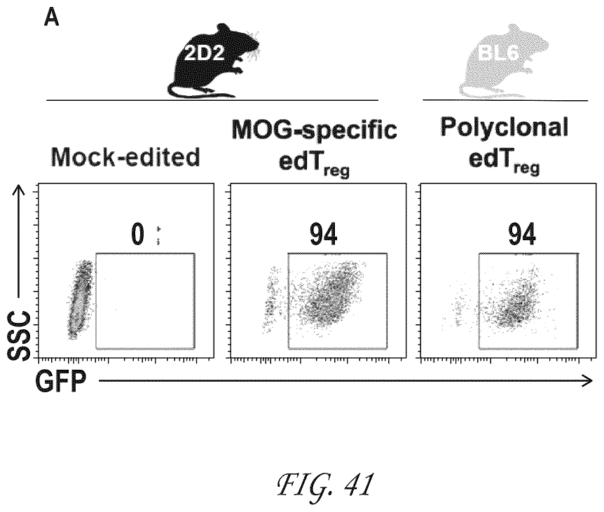

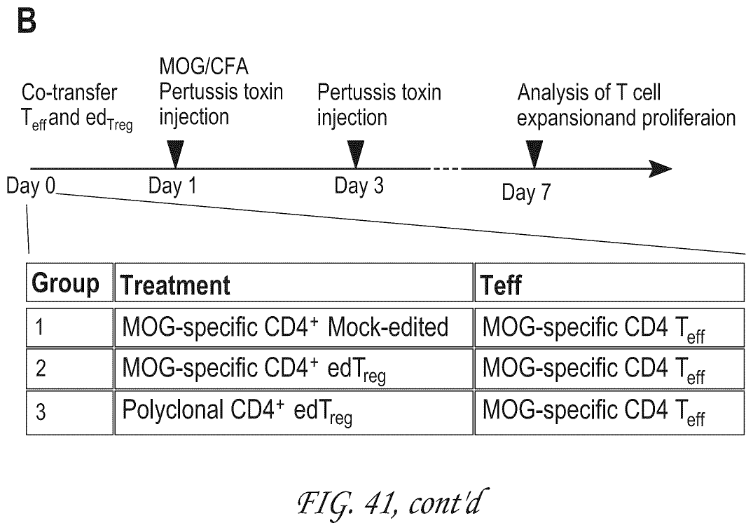

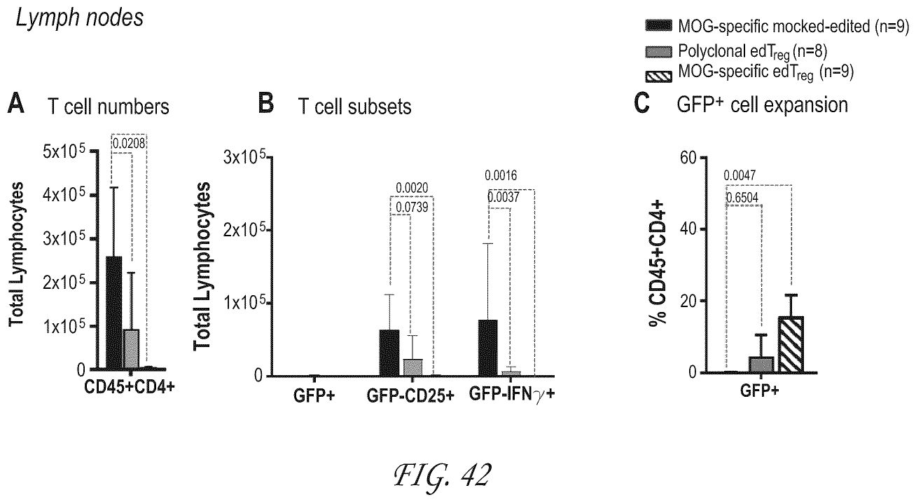

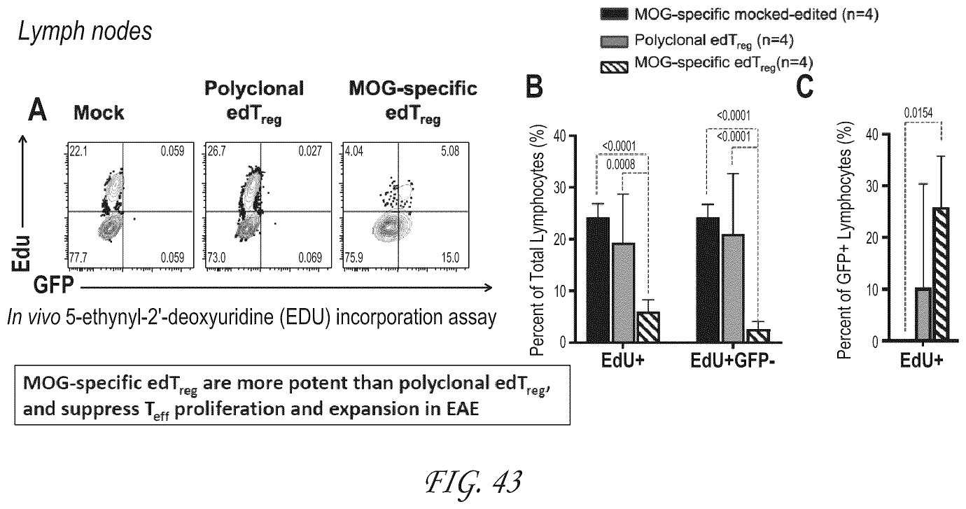

In order to investigate airT function in an antigen-specific in vivo setting, T cells for editing were selected from myelin oligodendrocyte glycoprotein peptide fragment 35-55 (MOG)-specific TCR-transgenic mice (C57B1/6-Tg(Tcra2D2,Tcrb2D2)1Kuch, abbreviated 2D2. MOG challenge of 2D2 transgenic mice leads to experimental autoimmune encephalomyelitis (EAE), a mouse model of multiple sclerosis. EAE in 2D2 mice is not controlled by endogenous 2D2 tTreg present within the central nervous system (CNS), possibly due to high levels of inflammatory cytokines produced by pathogenic T_{eff}. Adoptive transfer of antigen-specific 2D2 airT may suppress T_{eff} expansion in the periphery before these activated effectors migrate to the CNS (FIG. 38). To test this hypothesis, TALEN and AAV donor template reagents were designed that would mimic closely the GFP knock-in editing strategy used to generate the GFP+ human airT. After improved procedures for murine T cell stimulation and mRNA electroporation were designed, murine T cells were transfected with mRNA encoding TALEN pairs specific for the first coding exon of mouse Foxp3. Seven to 9 days post-transduction, approximately 80% of alleles contained indels based on colony sequencing of PCR-amplified gDNA (FIG. 39) indicating efficient target site cleavage. An AAV donor template was cloned that substituted mouse Foxp3 homology arms for the human sequences used in the previous HDR experiments; homology was proximal to, but not overlapping with, the mouse TALEN binding sites. With slight modifications of the conditions used for human CD4+ T cell editing, including using AAV5 capsid for donor template transduction, editing rates of approximately 25-30% (GFP+ cells) were consistently achieved using 2D2 mCD4+ T cells isolated from spleen and lymph nodes (LNs). GFP+ cells were phenotypically FOXP3+ CD25+ CTLA-4 (FIG. 40). CD4+ T cells were also isolated from 2D2neg littermate (C57BL/6) and edited, resulting in airT with a polyclonal pool of TCR specificities for comparison with 2D2 airT. Next, 3.0×10⁴ CD4+ T_{eff} cells from 2D2 mice, along with 3.0×10⁴ mock or airT, were adoptively transferred into lymphopenic RagI−/− mice. Recipient mice were challenged with MOG35-55 peptide in adjuvant followed by pertussis toxin to disrupt the blood-brain barrier. FIG. 41 shows the experimental timeline of cell transfer, immunization, and cell analysis. In this model, recipient animals develop symptoms of EAE (drooping tail progressing to tail, and then hind limb, paralysis) beginning at approximately 7-10 days post cell transfer. To assess effector T cell priming, recipient mice were euthanized at day 7 post-transfer and inguinal and axillary LNs were collected. The percentage of LN CD45+ CD4+ T cells were 2-fold lower in recipient of antigen-specific 2D2 airT compared to recipients of mock-edited cells, and 1.7-fold lower compared with recipients of polyclonal C57B1/6 airT. The absolute number of CD4+ CD45+ T cells was markedly reduced in both airT cohorts relative to the mock control, with 2D2 airT and C57B1/6 airT having 49-fold fewer and 18-fold fewer CD4+ CD45+ T cells, respectively. In all cohorts, the majority of CD45+ CD4+ cells were GFP−, and fewer GFP− T cells from mice receiving 2D2 edTreg expressed inflammatory markers CD25 and IFN-γ (FIG. 42). To determine if the observed effect was due to reduced T_{eff} proliferation, a subset of animals were injected with the thymidine analog 5-ethynyl-2′-deoxyuridine (EdU) 2 hours prior to sacrifice and its incorporation into gDNA was detected after a “click” reaction by flow cytometry (FIG. 43). 2D2 airT reduced the overall percentage of GFP− cells that had incorporated EdU by 22% and 18% relative to groups treated with mock or polyclonal airT, respectively. Importantly, GFP+ cells from 2D2 airT (˜25%) and, to a lesser extent, from C57B1/6 airT (˜10%) recipient cohorts incorporated EdU, consistent with the ability of tTreg to proliferate in vivo in response to self-antigen-stimulation. These combined findings show that murine airT function in vivo to restrain pathogenic T_{eff} priming and that antigen-specific airT exhibit greater activity and expansion in comparison with polyclonal airT.

### Example 6: airT Function in an Antigen-Specific Murine Model of Type 1 Diabetes (T1D)

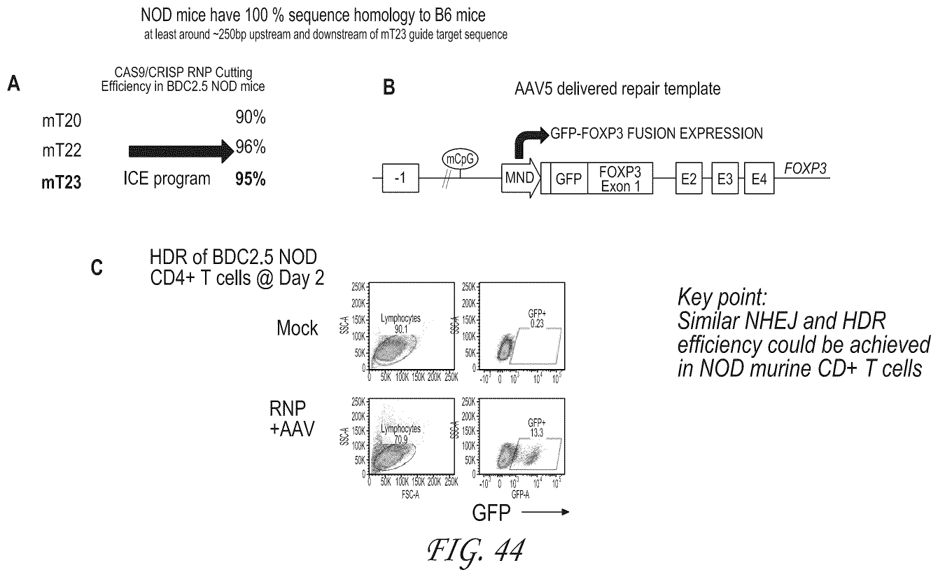

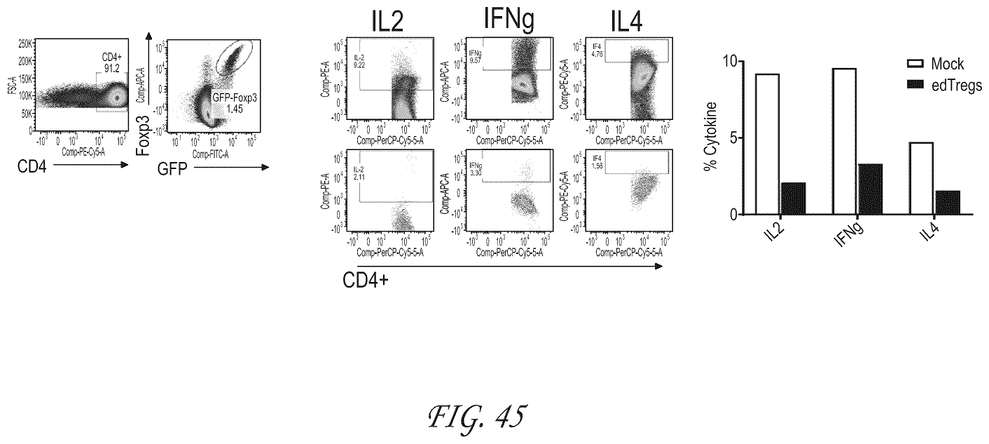

To investigate the efficacy of antigen-specific airT cells in an in vivo model of autoimmune T1D, a BDC2.5NOD-NSG adoptive transfer model was used as a tool to determine if Foxp3-edited antigen specific T cells could delay or reverse the onset of disease. NOD mice: (NOD/ShiLtJ strain) were used as a polygenic model for autoimmune Type 1 Diabetes (T1D). In this model, onset of diabetes is marked by moderate glycosuria and by a non-fasting plasma glucose higher than 250 mg/dl. Diabetic mice are hypoinsulinemic and hyperglucagonemic, indicating a selective destruction of pancreatic islet beta cells. It is currently the most widely used polyclonal autoimmune animal model to study spontaneous T1D. BDC2.5NOD mice: [NOD.Cg-Tg(TcraBDC2.5,TcrbBDC2.5)1Doi/DoiJ] carry both rearranged TCR alpha and beta genes from the cytotoxic CD4+ T cell clone BDC-2.5. Mature T cells in these mice express only the BDC2.5 TCR. On the NOD background, mice carrying the transgenes have a reduced incidence of diabetes relative to NOD/ShiLtJ controls. However, following transfer of CD4+CD25− BDC-2.5 T cells into immunodeficient recipient mice, recipient mice will quickly develop overt diabetes. In previous published studies, nTregs from antigen-specific BDC2.5NOD mice effectively prevent and reverse autoimmune diabetes in NOD mice relative to the nTregs from polyclonal WT NOD mice. Thus, in these experiments, these animal models were utilized as a tool to determine if Foxp3-edited antigen-specific T cells could delay or reverse the onset of autoimmune TID compared to nTregs from WT NOD. The ability to generate airT in NOD mice was tested. Previous studies demonstrate the ability to generate airT cells in murine CD4+ T cells from B6 mice using HDR editing of the Foxp3 locus (WO 2018/080541 and US 2019/0247443 which are each incorporated by reference in its entirety). Here, these studies were expanded and show that the same AAV donor templates have similar NHEJ and HDR efficiency in NOD murine CD4+ T cells (FIG. 44). Importantly, the Foxp3-edited BDC2.5NOD CD4+ T cells have a Treg phenotype expressing increased Foxp3, and less inflammatory cytokines as compared to mock cells (FIG. 45).

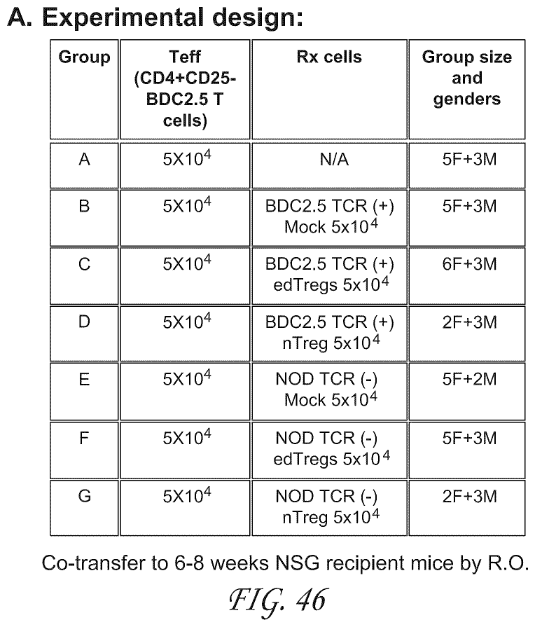

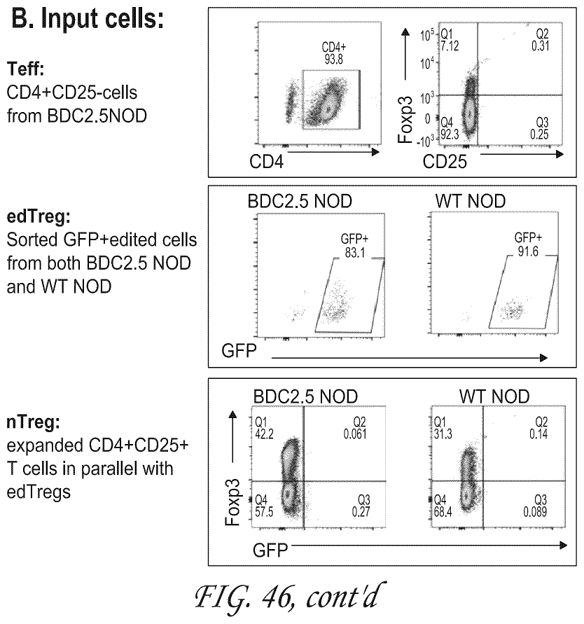

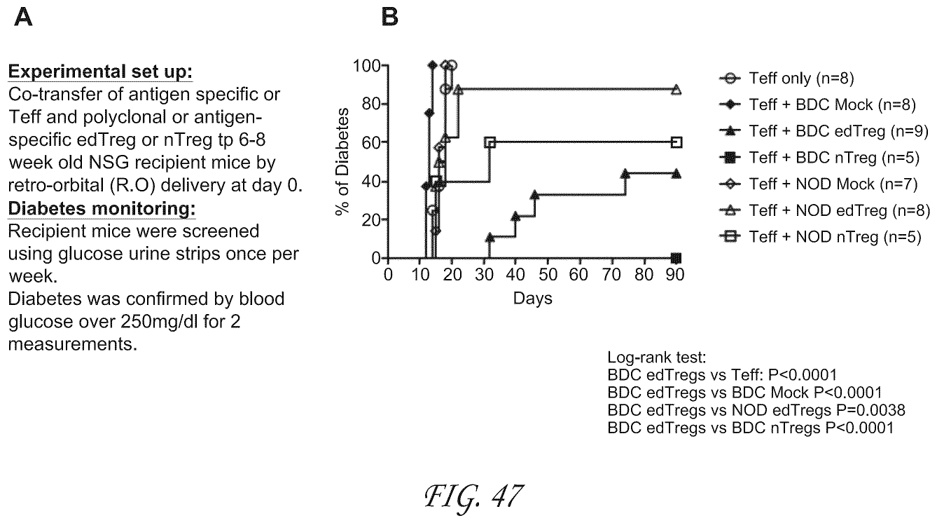

Antigen-specific airT cell function in an NSG adoptive transfer model may be more efficacious than non-antigen specific airT cells at delaying or reversing the onset of autoimmune T1D. nTregs were included in this study as this population has previously been shown to reduce the onset of T1D. The experimental design and the phenotype of the input T_{eff}, airT and nTreg cells are shown in FIG. 46. Like nTreg, airT lead to a reduction in percentage of diabetes compared to mock airT or animals receiving T_{eff} only (FIG. 47). Importantly, administration of BDC airT leads to a statistically significant decrease in percentage diabetes compared to polyclonal NOD airT. This finding demonstrates that Ag-specific airT more effectively prevent diabetes development compared with polyclonal airT. Of note, previous studies have shown that the N-terminal GFP-FOXP3 fusion protein functions as a hypomorph and can actually accelerate autoimmune diabetes within the immunocompetent NOD background. Consistent with this idea, while antigen-specific nTregs performed better than antigen-specific airT in these studies, a significant suppressive effect was still observed with airT expressing GFP-FOXP3 fusion protein. Testing the airT expressing FOXP3 without the N-terminal GFP fusion (including airT cells expressing a clinically relevant cis-linked LNGFR selectable marker; see below), an improved protective effect would be expected.

Taken together, these findings clearly showed that in the BDC2.5 NOD T cell into NSG adoptive transfer model, Ag-specific airT function to prevent diabetes development as compared with polyclonal airT.

### Example 7: Engineering AAV Donor Template Design to Generate airT Product with LNGFR Selectable Marker

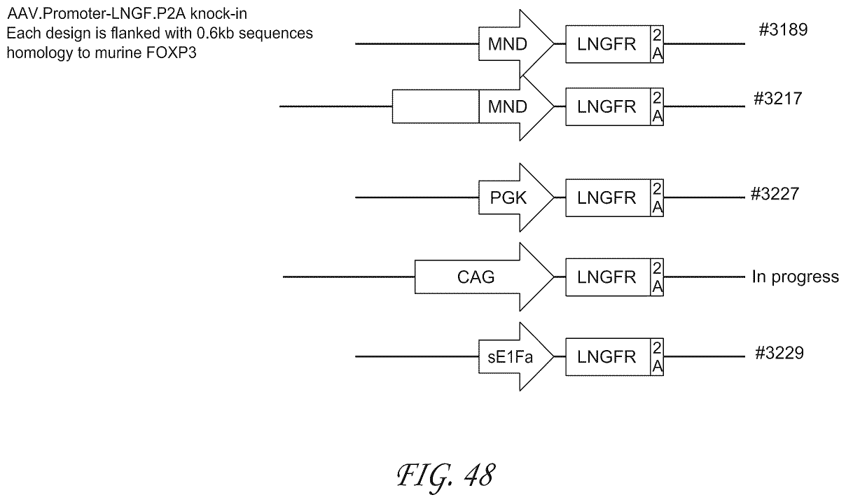

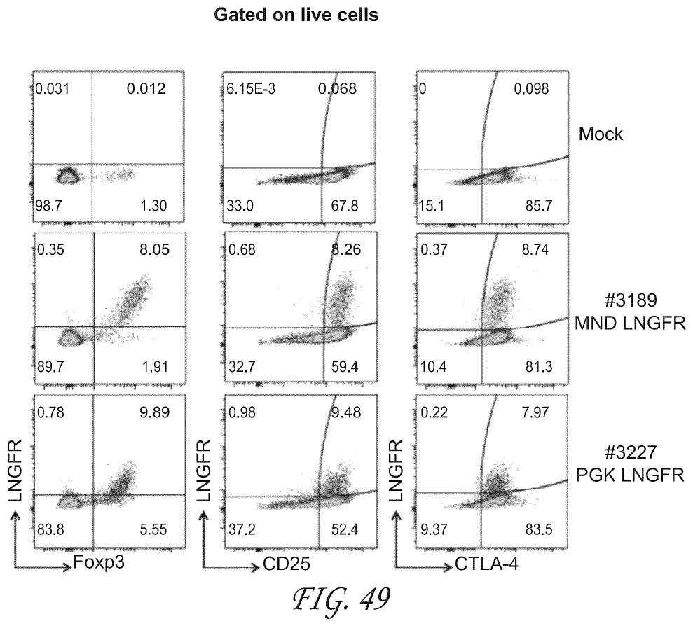

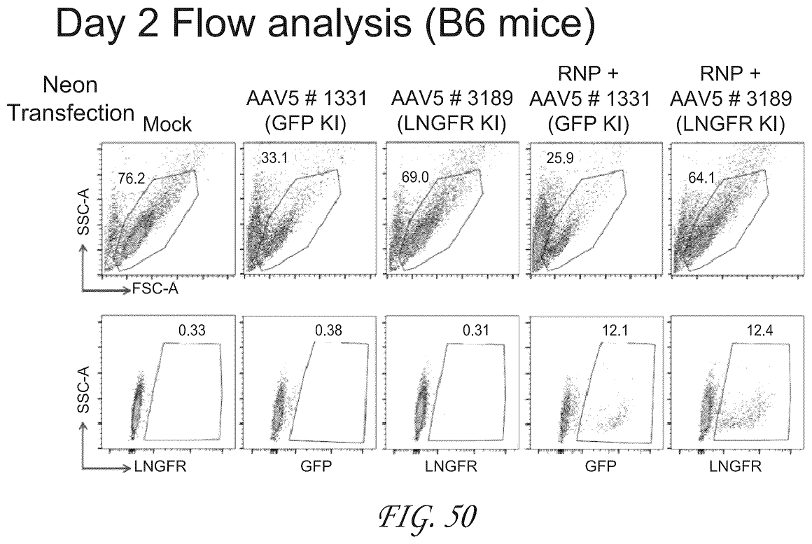

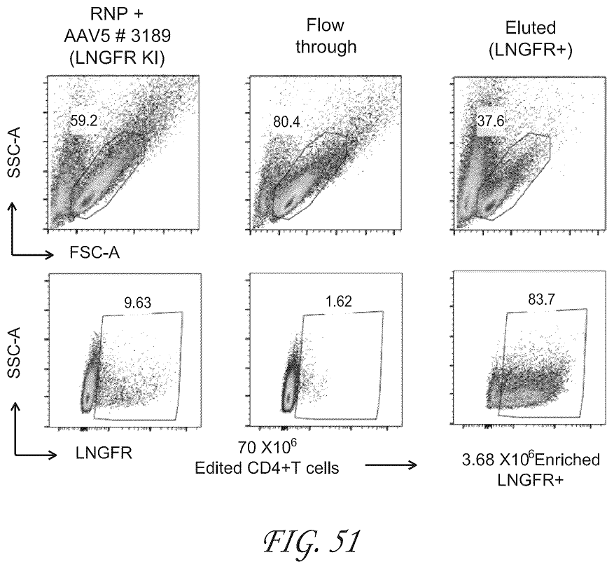

The ability to enrich murine cells following editing is important for generating sufficient numbers of edited cells to perform in vivo experiments without sorting. To address this, a cis-linked LNGFR selectable marker has been developed for use in purification of murine edTeg products. FIG. 48 shows the design of repair templates used in murine Foxp3 editing. Each template contains a LNGF.P2A knock-in (ki) but varies in terms of the promoter. In addition, the presence and absence of 07UCOE was tested with MND promoter. Following transfection of RNP+AAV5 #1331 (MND-GFP), #3189 (MND-LNGFR), and #3227 (PGK-LNGFR) in B6 CD4+ T cells, the editing efficiency of GFP and LNGFR KI was very similar (FIG. 49, FIG. 50). In addition, an 8.7-fold enrichment of LNGFR+ cells was demonstrated using an anti-LNGFR microbeads and magnetic field separation (FIG. 51). These data suggest LNGFR can successfully be used as a selection and enrichment method of murine CD4+ T cells.

### Example 8—Methods

### Foxp3 Editing

CD4+ T cells were isolated from PBMCs using MACS CD4+ T cell isolation kit and activated with CD3/CD28 activator beads (1:1, bead to cell ratio) and IL-2, IL-7, and IL-15. Beads were removed after 48 hr activation and cells were rested for another 16-24 hr. For Foxp3 editing using Cas9/CRISPR, cells were transfected by electroporation with RNP complex combined with Cas9 and guide RNA and then transduced with AAV template (AAV FOXP3 ex1.MND-LNGFRki). For Foxp3 editing using TALEN nuclease, cells were transfected by electroporation with TALEN RNA targeting FOXP3, followed by transduction with AAV template (AAV FOXP3 ex1.MND-GFPki). Cells were expanded in media with IL-2 after editing.

### Generation of airT Cells with Islet-TCR

CD4+ T cells were isolated from PBMCs and activated with CD3/CD28 activator beads and IL-2, IL-7, and IL-15. Transduction with lentiviral vectors encoding GAD65 or IGRP specific TCRs (4.13, T1D2, T1D4, T1D5-1, or T1D5-2) was performed at MOI 10 with protamine sulfate after 24 hr activation. Beads were removed after total 48 hr incubation from the initial activation. Cells were rested for another 16-24 hr and then edited. For Foxp3 editing, cells were electroporated with RNP complex combined with Cas9 and guide RNA and then transduced with AAV FOXP3 ex1.MND-LNGFRki template. Cells were expanded in media with IL-2 after editing and editing rate and TCR-transduction were measured 3-4 days after editing. airT cells were enriched by LNGFR expression using MACSelect LNGFR beads, aliquoted, and frozen down for future experiments.

### Generation of Antigen-Specific airT Cells

For generating T cells specific for Flu or Tetanus, CD4+CD25− cells were isolated from PBMCs and co-cultured with APC (irradiated autologous CD4−CD25+ cells) and MP, HA, and TT peptides in the presence of IL-2. CD4+ T cells were stimulated twice with peptides and APC for 9 days and then activated with CD3/CD28 beads for Foxp3 editing using TALEN and AAV FOXP3 ex1.MND-GFPki template. 3 days after editing, GFP+ cells were sorted by flow and expanded with CD3/CD28 beads. Beads were removed after 7 days expansion and airT cells were harvested for suppression assay after another 4 days incubation.

For generating islet-specific T cells by peptide stimulation, CD4+CD25− cells were isolated from PBMCs and co-cultured with APC and a pool of islet-specific antigens (total 9 peptides from IGRP, GAD65, and PPI). After 2 weeks of expansion, cells were harvested and stained with tetramers specific for 9 islet peptides for sorting. Sorted islet-specific CD4+ T cells were activated with CD3/CD28 activator beads for Foxp3 editing using Cas9/CRISPR and AAV FOXP3 ex1.MND-LNGFRki template. Cells were stained by tetramers and analyzed by flow 3 days after editing.

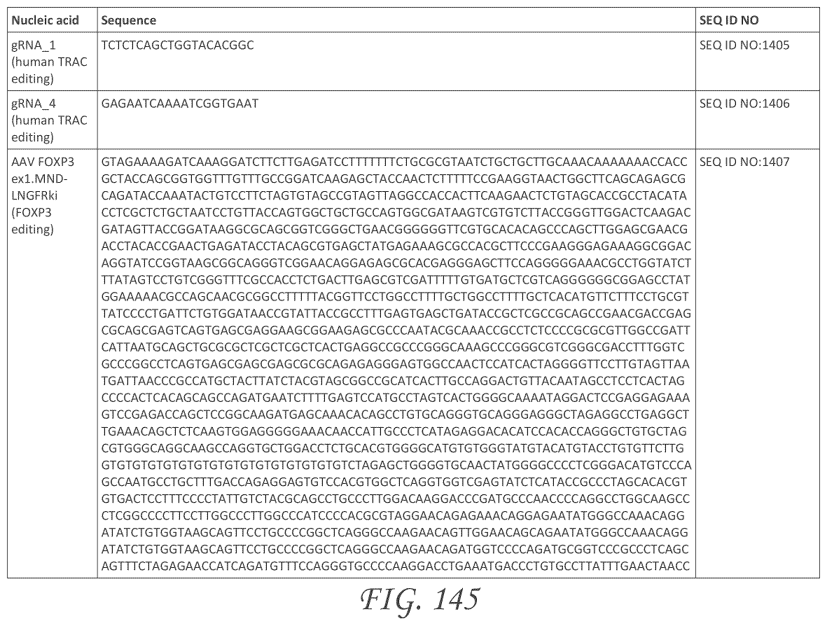

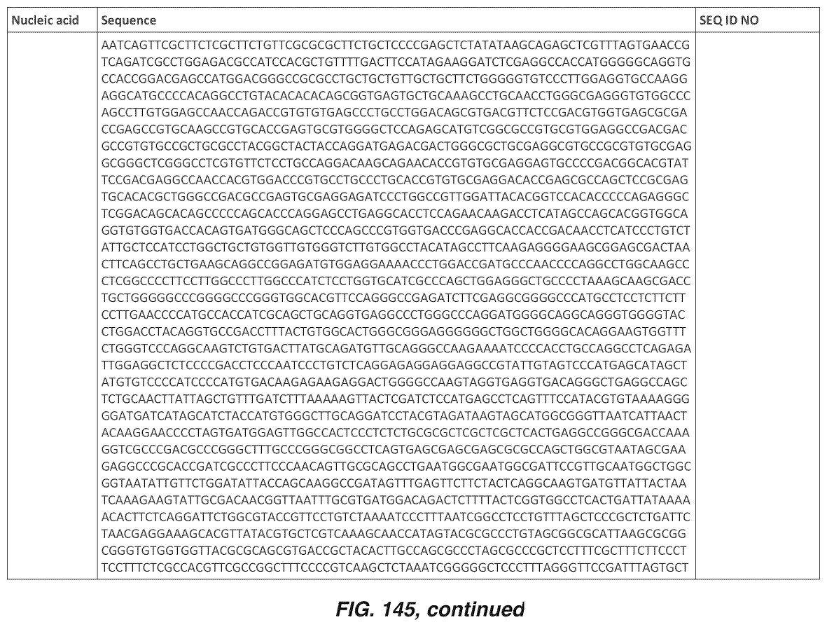

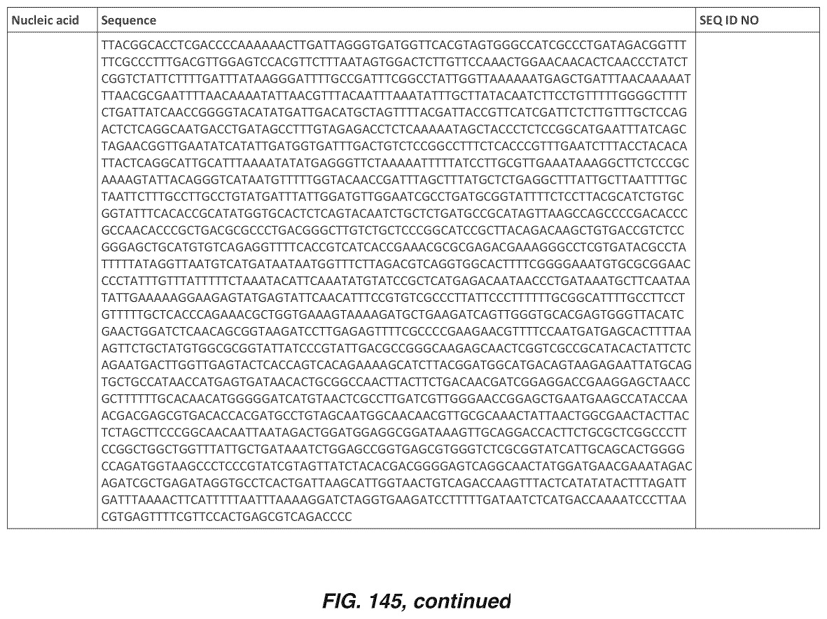

Certain nucleic acid sequences useful with embodiments provided herein are listed in the TABLE depicted in FIG. 145.

### Example 9—Comparison of airT with T Cells Expressing Lentiviral (LV)-Delivered FOXP3

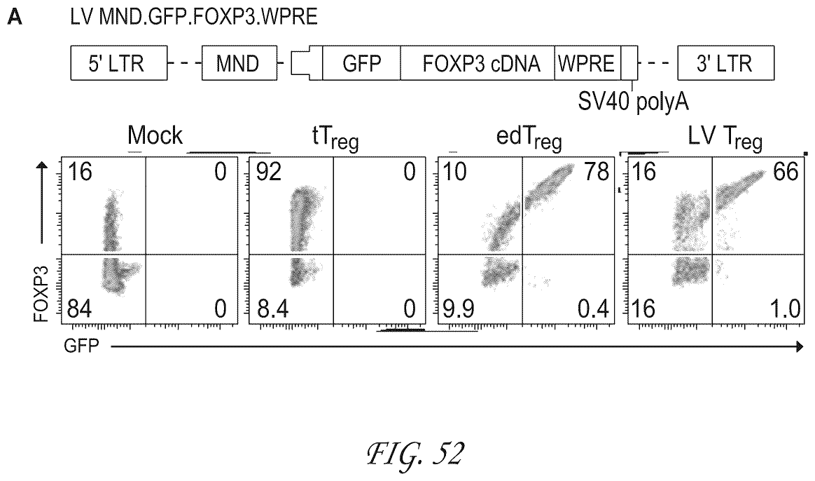

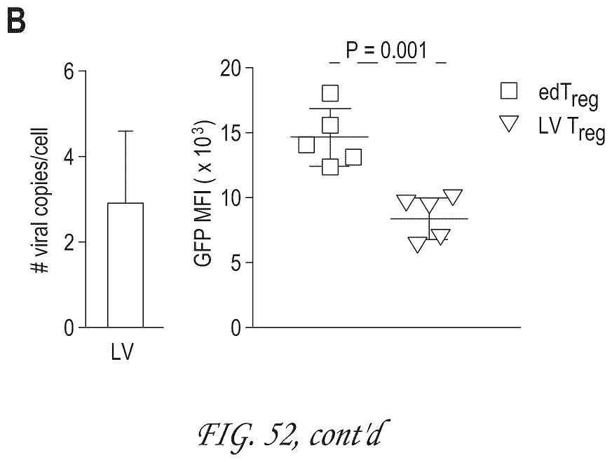

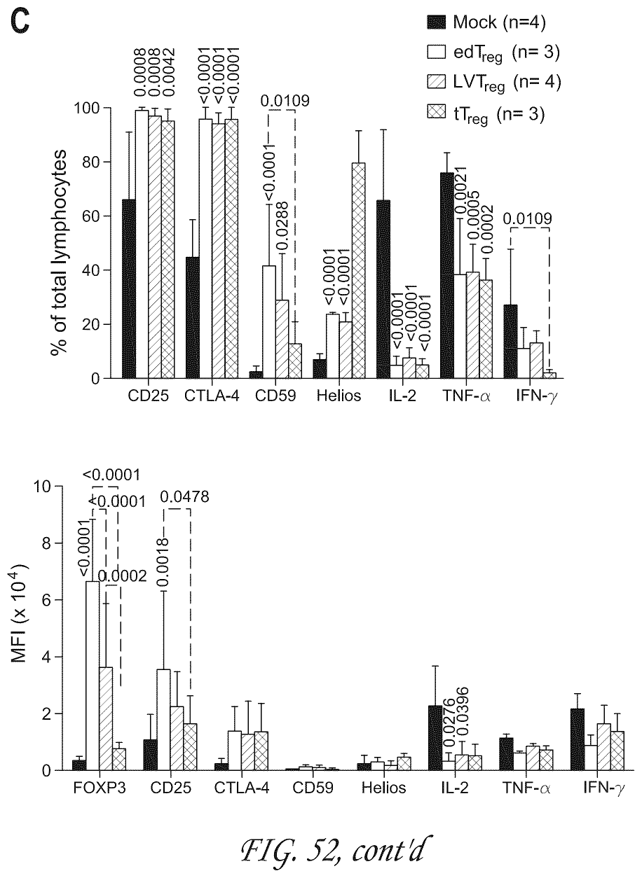

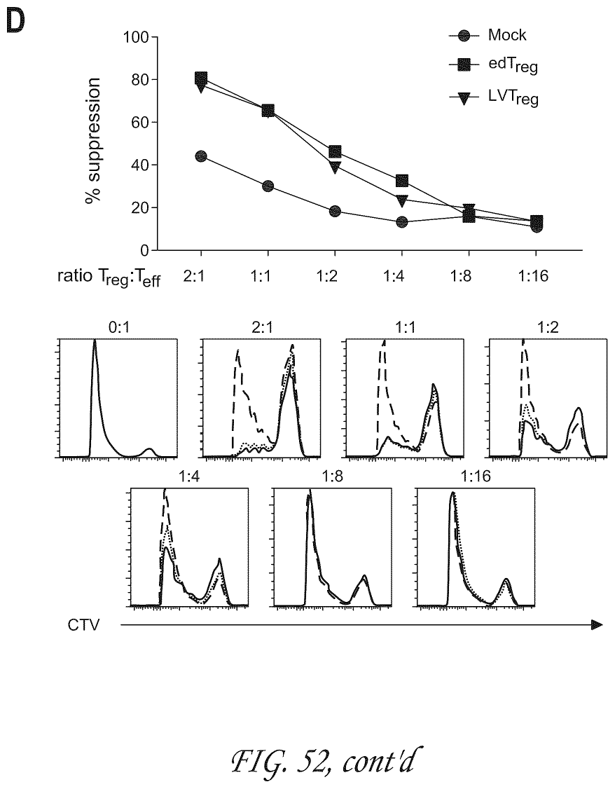

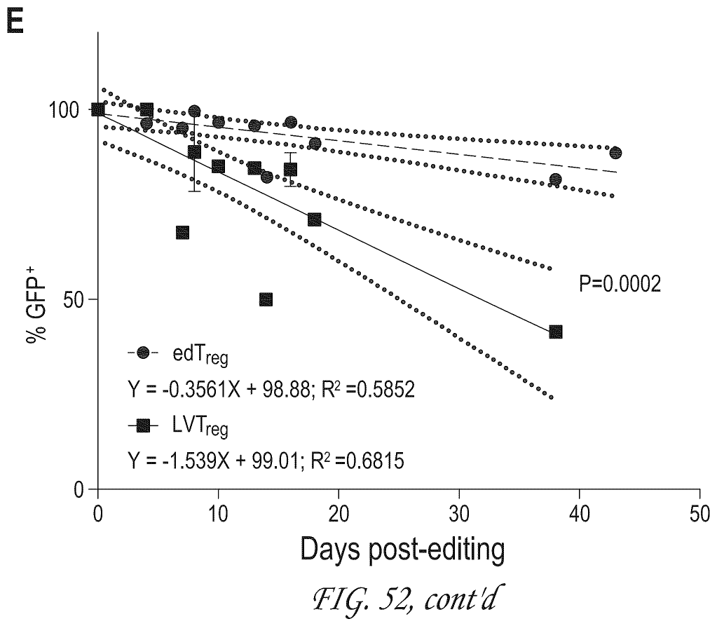

LV transfer of a FOXP3 cDNA expression cassette into conventional T cells has been shown to confer a Treg-like phenotype and suppressive characteristics in vitro and in vivo (Allan et al. Mol Ther 16:194-202 (2008); Passerini et al. Sci Transl Med 5:215ra174 (2013)). As a comparison for the presently disclosed editing strategy, a LV construct was generated to deliver a cDNA encoding the same GFP-FOXP3 fusion protein made by the airT cells (FIG. 52A). The gene editing and viral transduction procedures produced similar proportions of GFP+FOXP3+ cells (FIG. 52A). LV-treated cells (LV Treg) had an average of 3.0 lentiviral copies per GFP+ cell genome; airT have only one targeted insertion per cell in this experiment, edited T cells from male donors. Despite their copy number differences, the MFI of GFP+ cells were consistently lower in LV Treg than in airT (FIG. 52B), consistent with more efficient expression from a genomic vs. cDNA context, or LV integration in transcriptionally less permissive loci. Both methods of enforcing FOXP3 expression skewed the T_{eff} towards tTreg phenotypes, including upregulation of CD25, CTLA-4, and Helios, and down-regulation of IL-2, TNF-α, and IFN-γ (FIG. 52C). Except for FOXP3, the percentage of cells expressing regulatory T cell markers, as well as the mean expression (as assessed by MFI) per cell, were similar between airT and LV Treg. airT and LV Treg exhibited a similar ability to suppress polyclonal T_{eff} proliferation in vitro (FIG. 52D). Importantly, however, FACs purified LV Treg cells lost GFP expression over 5 weeks in culture compared to airT (FIG. 52E; starting % GFP+ >99% for both). This latter finding demonstrates that HDR editing more effectively maintains high-level FOXP3 expression compared with LV delivery using the identical promoter construct. These findings are unexpected based upon previous reports using LV delivery of FOXP3 and support the concept that HDR editing of the FOXP3 locus provide a more stable platform for sustained expression of FOXP3 in CD4 T cells.

### Example 10—Dual-Editing Strategies

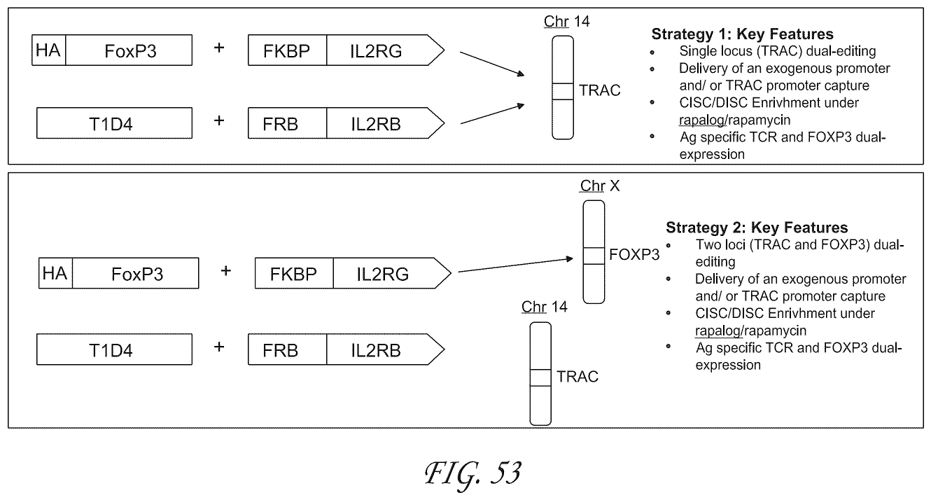

FIG. 53 provides an overview of the HDR gene-editing strategies developed to generate antigen-specific airT via HDR-editing-only approaches. These novel approaches eliminated the requirement for use of LV for TCR delivery and are designed to generate airT that concurrently: a) lack endogenous TCR expression; b) express an islet-specific T1D (or another disease-relevant, antigen-specific TCR); and c) can be enriched in vitro and in vivo using the novel CISC/DISC IL-2 platform. As shown in FIG. 53, strategies were developed to achieve the above goals by either targeting a single locus (TRAC) or two-loci (TRAC and FOXP3). Successful application of both of these strategies is illustrated below.

FIG. 54 provides a schematic overview and construct number designation for each of the AAV HDR donor constructs used in the studies described below for either single and two locus dual-editing approaches for generation of Ag-specific edTreg; with or without IL2-CISC/DISC selection capacity.

### Dual Editing of Human CD4+ T Cells—Examples of Single Locus Approach

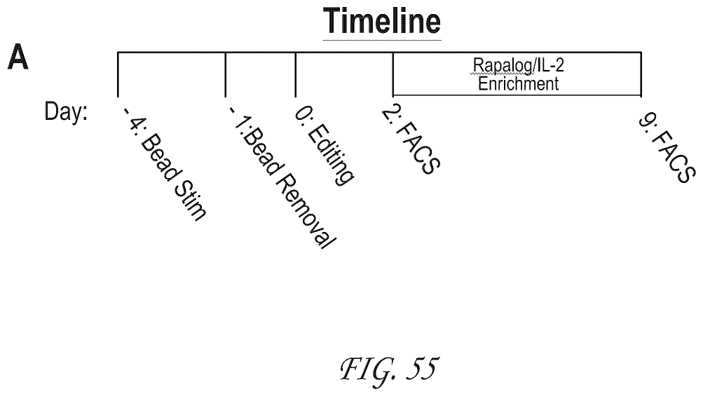

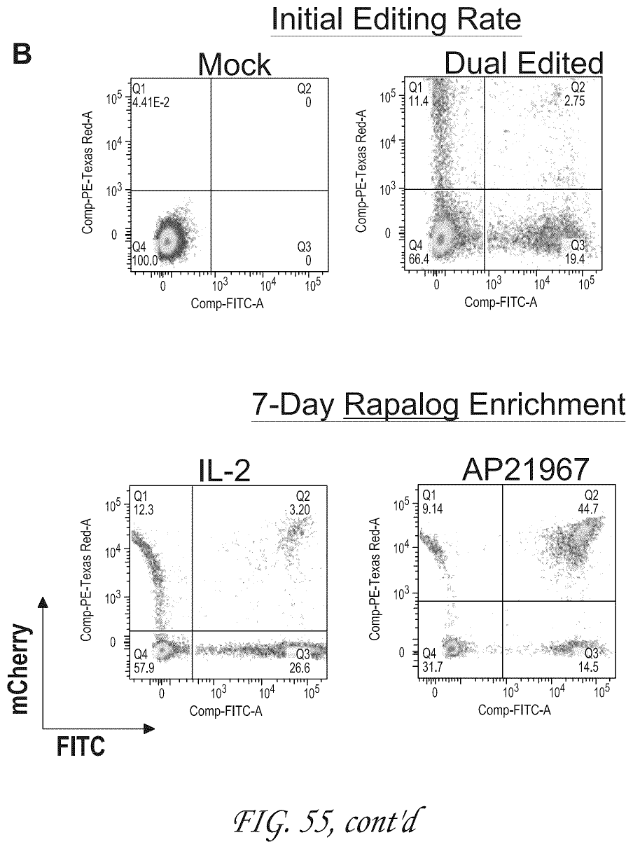

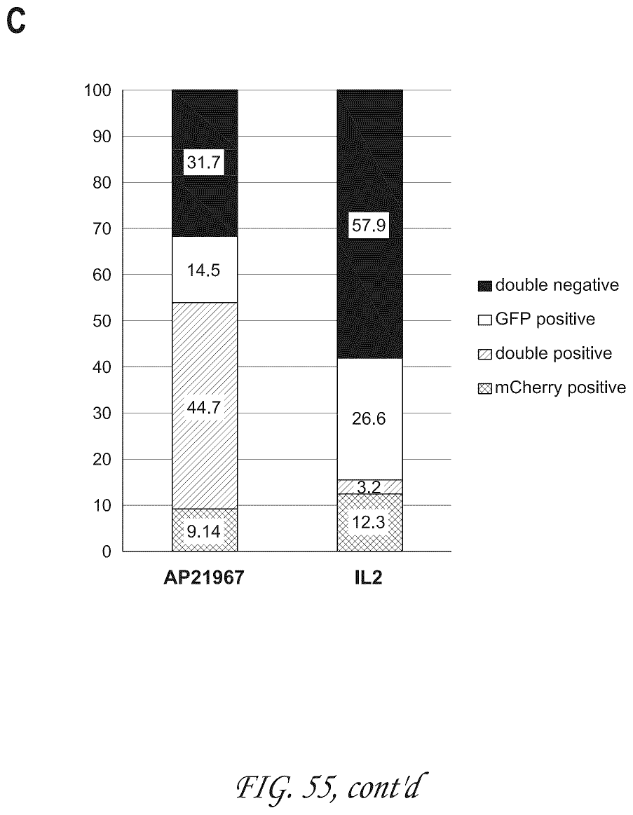

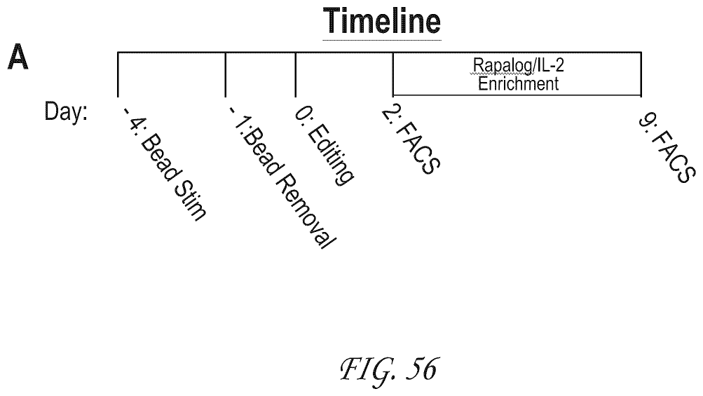

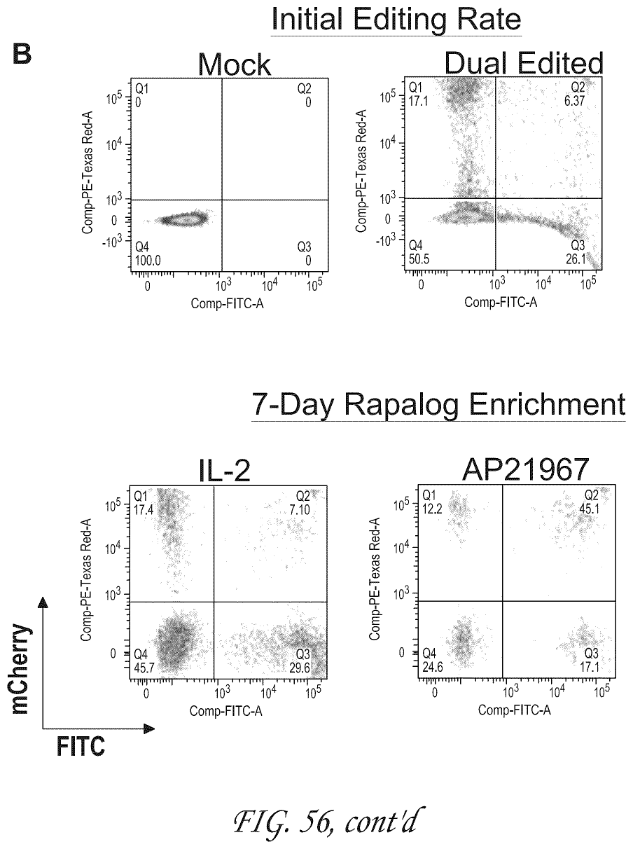

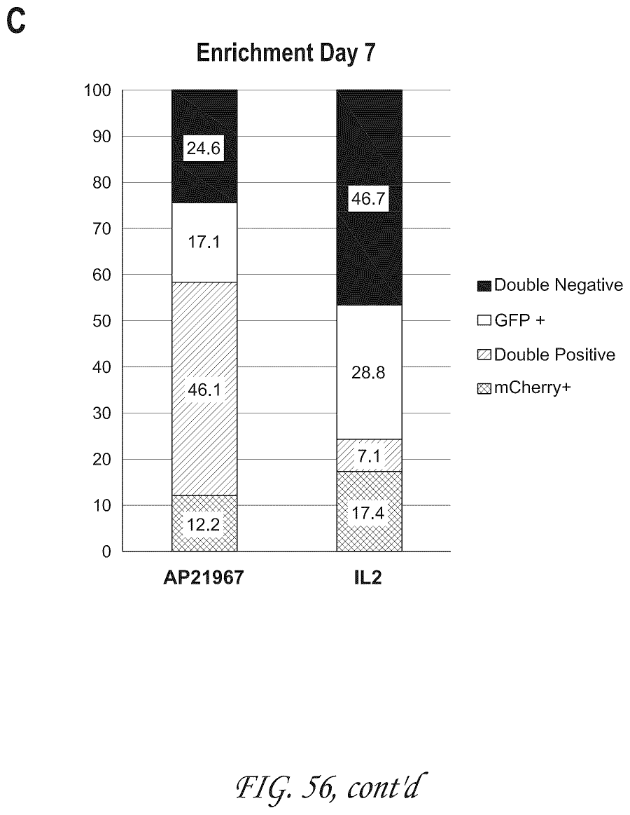

FIG. 55 and FIG. 56 relate to reproducibility between experiments and variance between donors. Two donors were edited with AAV #3207 (MNID.GFP.FRB-IL2RG) and #3208 (MNID.mCherry.FKBP-IL2RG), used in previous experiments and compared the repeat experiments to the original data. In these experiments, both HDR repair templates are targeted to a single sgRNA cut site within the first exon of the TRAC locus. The percent dual-editing (incorporation of both the GFP and mCherry split-CISC cassettes in a single cell) of donor R003657 was 2.75% (FIG. 55) which is similar to results observed in two prior data sets (1.44% and 1.1% for donor R003657). Percent dual-editing of the second donor (R003471) was 6.78% (FIG. 56), also similar to the 6.4% observed in the original data set. Both donors were male, Caucasian of similar age. Overall, the editing rate varied between donors, but each donor had similar editing rates between experiments, suggesting that variability is based on how well the donor can be edited and not the between different experiments of the same donor. Importantly, using edited T cells derived from both human donors, dual-edited cells were successfully enriched in the presence of a heterodimer-inducing rapamycin analog (Rapalog, AP21967) to a similar level to what was previously observed. This study yielded 44.7% and 46.1% GFP/mCherry double positive cells for donors R003657 and R003471 respectively after 7 days of Rapalog enrichment (FIG. 55, FIG. 56). As expected, there was no enrichment in the presence of IL-2.

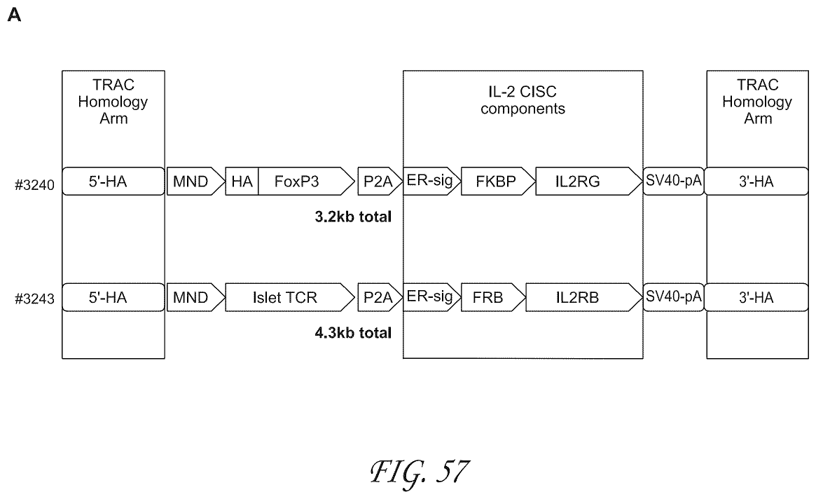

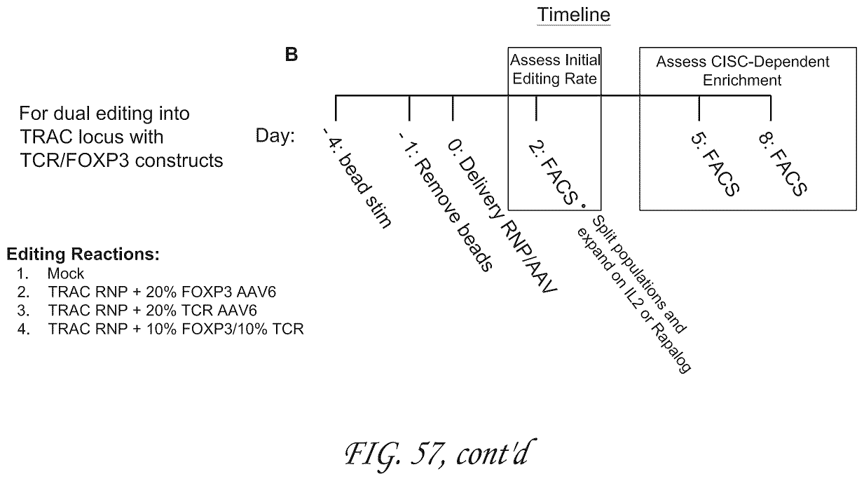

The results of these studies demonstrate that incorporation of the IL-2 split-CISC in dual HDR editing strategies provides a means of efficient selection and enrichment of dual edited cells which is reproducible between donors and repeat experiments. Following successful dual editing using the MND.GFP.FRB.IL2RB (#3207) and MND.mCherry.FKBP.IL2RG (#3208) cassettes, and enrichment of dual edited cells using Rapalog, constructs were generated that can be used in introducing HA tagged FOXP3 and a pancreatic islet antigen-specific TCR (T1D4) in combination with the IL-2 CISC components (#3240 and 3243 respectively) to generate antigen-specific FOXP3+ edTreg cells (FIG. 57).

After replacing GFP with T1D4 and mCherry with HA-tagged FOXP3, respectively, the MND.HA.FOXP3.FKBP.IL2RG (#3240) and MND.T1D4.FRB.IL2RB (#3243) constructs were used to test the initial editing rates and expansion of FOXP3 expressing T1D4 positive human edTregs. Constructs are shown in FIG. 57(A) and the timeline and editing conditions for this experiment are shown in FIG. 57(B). Despite lower initial editing rate compared with using the GFP/mCherry-CISC constructs (FIG. 55, FIG. 56), 2.75% and 6.37% double-positive GFP/mCherry cells versus 0.65% double-positive FOXP3/T1D4 cells, (FIG. 57), the double-positive FOXP3/T1D4 cells could be significantly enriched. In the presence of Rapalog, FOXP3/T1D4-positive cells enriched from 0.65% to 11% after 8 days of treatment compared to 1.1% with IL-2 treatment (FIG. 57(C)). The reduction of initial editing rate with these constructs compared to the Split-CISC constructs containing GFP and mCherry (#3207 and #3208) could potentially be due to the increased size of the T1D4 HDR template. MND.T1D4.FRB.IL2RB (#3243) is 4.3kb, which was significantly larger than MND.mCherry.FKBP.IL2RB (#3208) at 2.7kb.

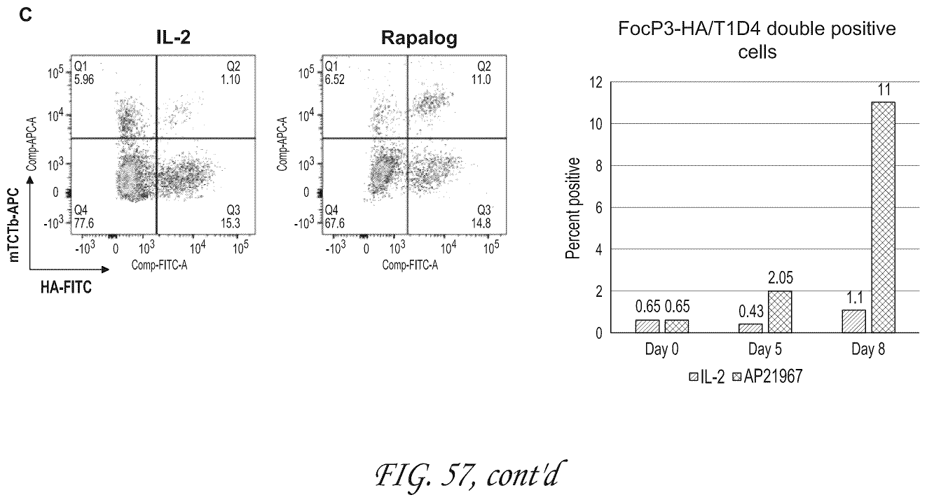

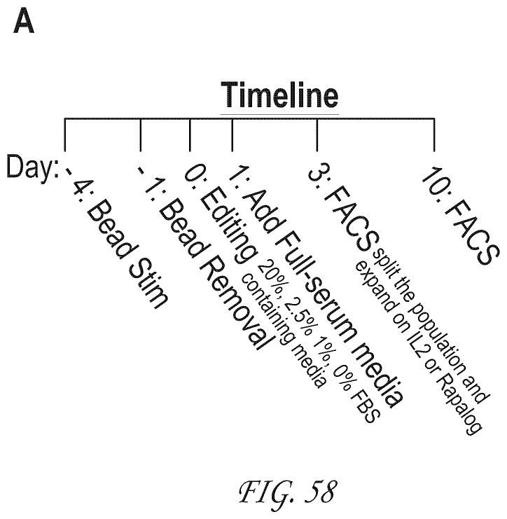

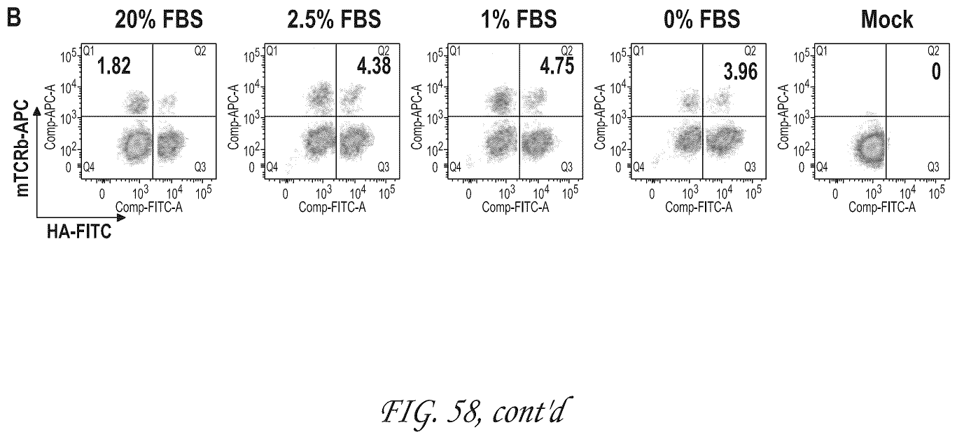

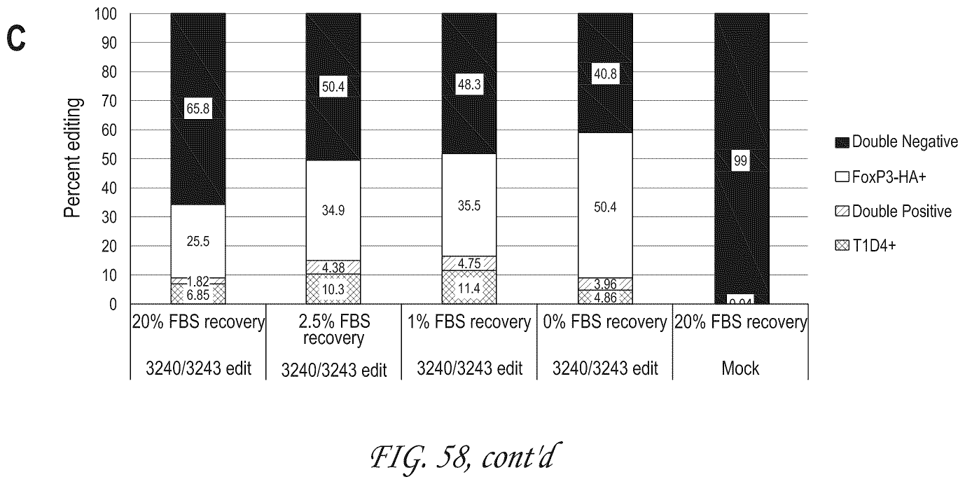

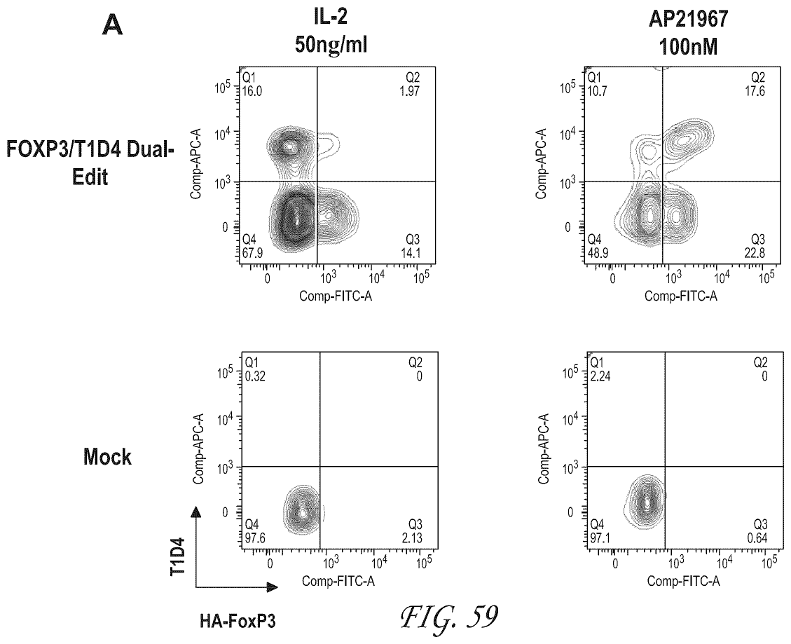

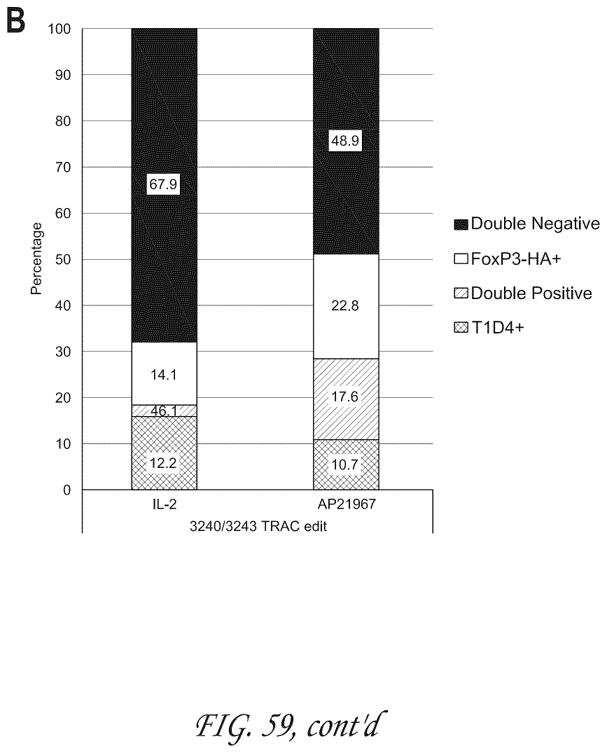

In order to obtain sufficient numbers of edited cells for therapeutic use, it may be important to increase the rate of editing and/or enrichment of FOXP3/TCR dual-positive cells. To improve upon initial editing rates, conditions were modified by varying the serum concentration during the editing phase. FIG. 58 and FIG. 59 show results from a dual-editing experiments using AAV constructs MND.HA.FOXP3.FKBP.IL2RG (#3240) and MND.T1D4.FRB.IL2RB (#3243) comparing four different concentrations of serum during the editing phase of human CD4+ T cells. The resulting FACS analysis demonstrates an improvement of initial editing rate from 1.8% with 20% FBS to 3.96%-4.75% in lower or no serum (FIG. 58). Importantly, the resulting enrichment was nearly 10-fold in cells which recovered in 2.5% FBS, increasing from 1.8% to 17.6% double-positive cells in 7 days of Rapalog treatment, compared to 1.97% with IL-2 treatment (FIG. 59).

In summary, FIGS. 55-59 demonstrate the capacity to achieve efficient levels of dual-HDR editing at the TRAC locus leading to generation of antigen-specific airT that exhibit enrichment using the IL-2 CISC platform.

### Dual Editing of Human CD4+ T Cells—Examples of Two Loci Approach

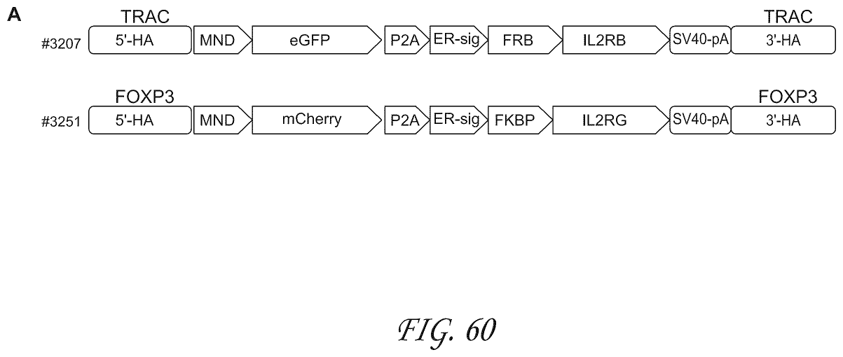

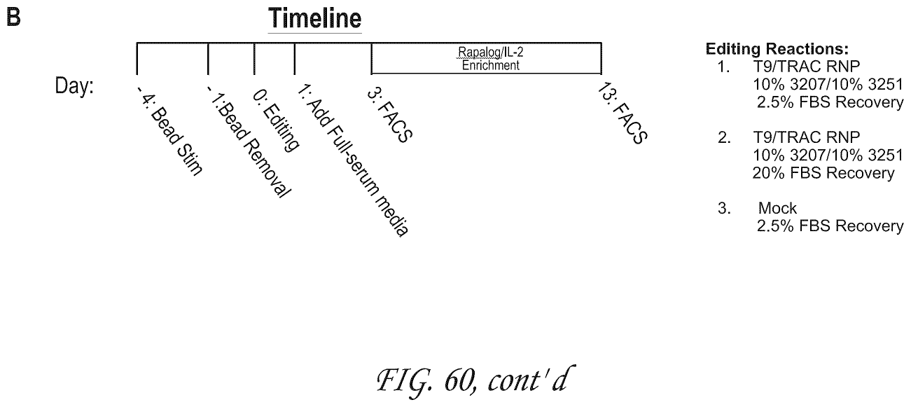

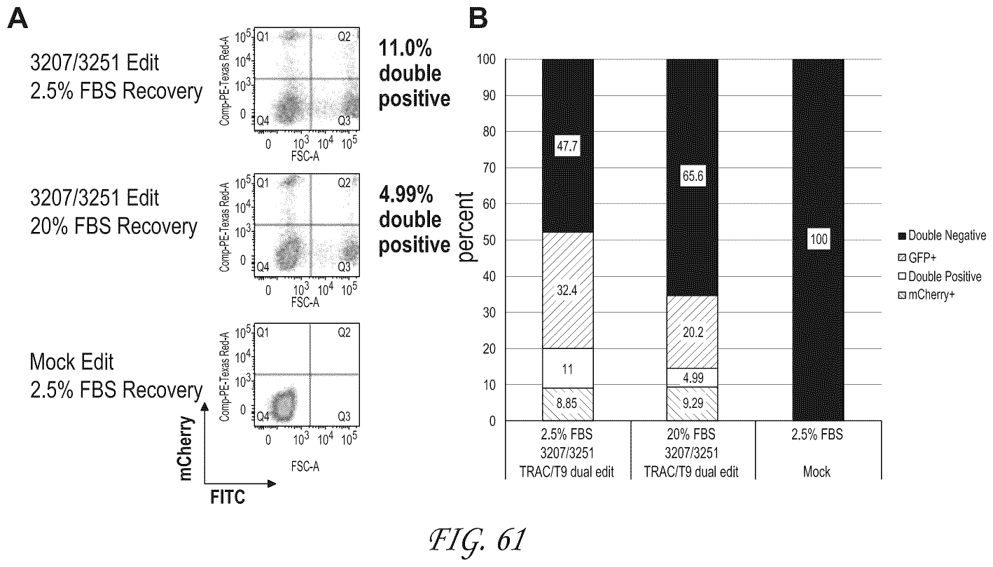

As an alternative dual-editing strategy for generating antigen-specific airT product containing IL-2 CISC components, constructs targeting to the TRAC and FOXP3 loci were developed for two-locus editing. As shown in schematic on FIG. 53, instead of having two constructs targeted to the TRAC locus, one construct is targeted to TRAC locus and the other is targeted to FOXP3 locus. This approach might lead to an improved dual-editing rate and also permit coordinated use of multiple existing strategies to mediate sustained FOXP3 expression. To test this, constructs were developed that would allow easy tracking of successfully edited cells. MND.mCherry.FKBP.IL2RG (#3251) and MIND.GFP.FKB.IL2RB (#3207) cassettes with FOXP3 and TRAC homology arms, respectively, were used to test the ability to generate dual-edited cells with stable expression of both mCherry and GFP linked to the IL-2 CISC. The constructs used and time line for editing, cell expansion and analysis are shown in FIG. 60. For this experiment, high serum and low serum editing conditions were compared since the single-loci editing suggested that lower serum concentrations resulted in higher dual editing rates (FIG. 58). The resulting FACS analysis demonstrates successful editing using the two-loci strategy with both serum conditions (FIG. 61). As with the single-locus editing, 2.5% serum media during the editing phase significantly improved dual editing at day 3 (4.99% editing rate with 20% serum vs 11.0% with 2.5% serum). The cells edited in 2.5% serum containing medium were then expanded with either IL2 or Rapalog (AP21967) for 10 days. FIG. 62 shows the robust enrichment in the presence of Rapalog for a total of 59% mCherry/GFP double-positive cells.

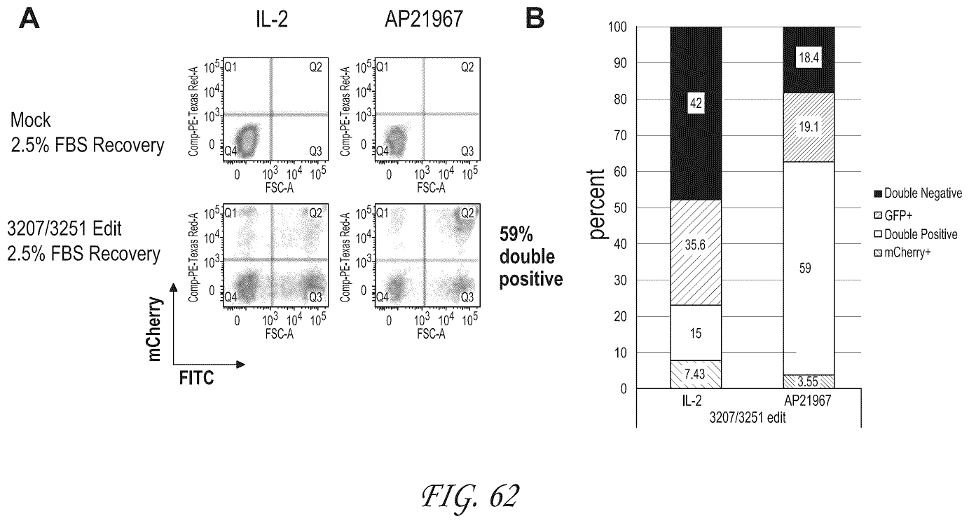

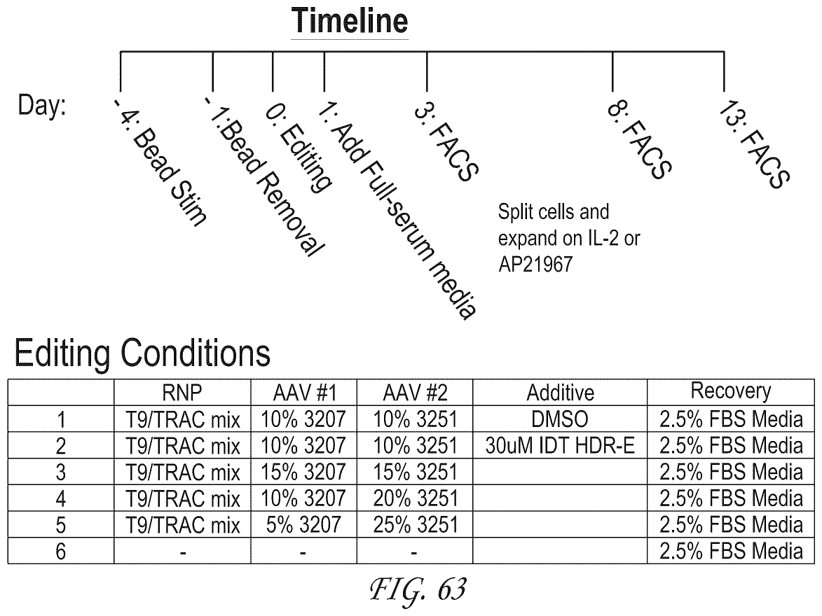

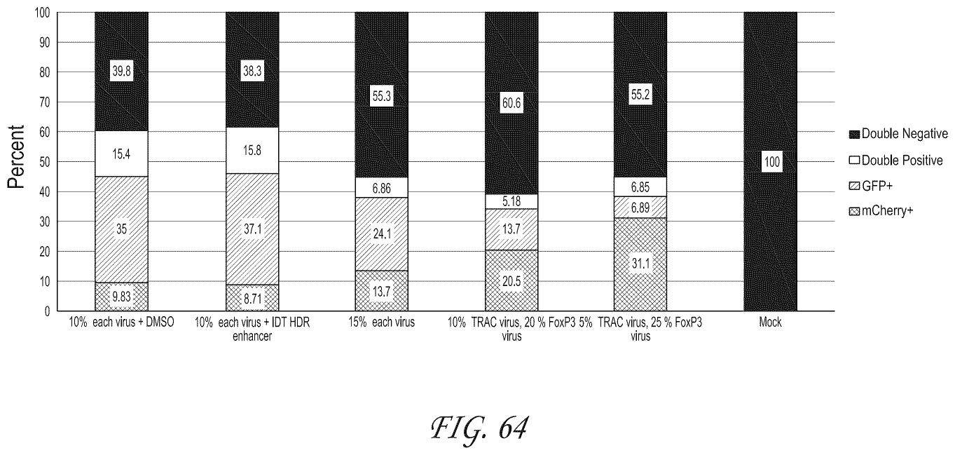

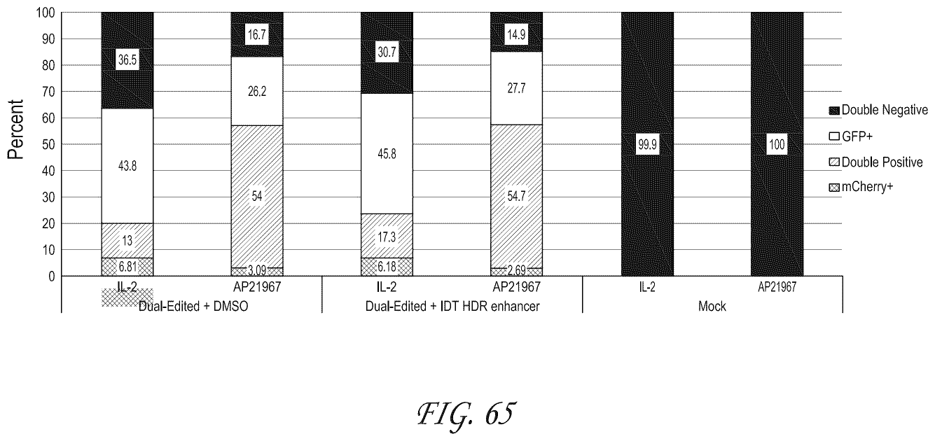

The reproducibility between experiments was tested and alternative editing conditions were explored in an attempt to further increase the initial editing rates. FIG. 63 outlines editing conditions and the time-line for editing, cell expansion and analysis. For this experiment, 2.5% serum containing medium was used in the editing phase for all conditions. The percentage virus by volume was varied in the reaction and editing was compared using AAV #3207 and #3251 in the presence and absence of an HDR enhancer (HDR-E). FIG. 64 shows histograms from flow data in each condition 3 days post editing. The editing rate with 2.5% serum containing medium was similar in this study compared to the prior experiment shown in FIG. 61 (15.4% double-positive GFP/mCherry cells compared 11% double-positive cells respectively). In addition, Rapalog enrichment of the dual-edited population resulted in 54% GFP/mCherry double-positive cells (FIG. 65), similar to previous data and demonstrating reproducibility between experiments. Although the presence of HDR-E did not affect the initial editing rate, the % virus in the reaction did impact editing outcomes (FIG. 64). The results showed that 10% culture volume of each virus was optimal compared to 15% each, or any of the other combinations with a total of 30% virus.

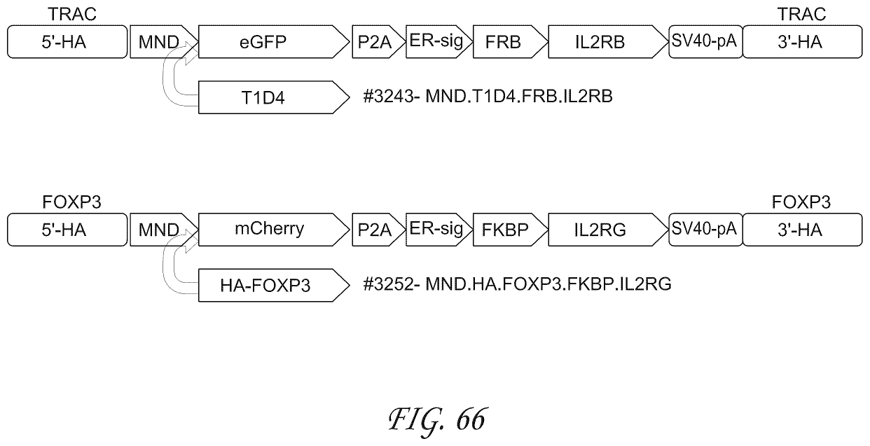

The results of these studies demonstrate that presently disclosed two-loci dual-editing strategies can be used to introduce the IL-2 split-CISC cassette and lead to efficient enrichment of dual-edited cells using Rapalog. Having been successful using this approach for generation and enrichment of dual-edited cells, constructs were designed and cloned for expression of FOXP3 and a pancreatic islet antigen-specific TCR (T1D4) in combination with IL-2 CISC components by targeting the FOXP3 and TRAC loci, respectively. These and a range of other HDR donors are used to generate antigen-specific FOXP3 airTcells (FIG. 66).

### In-Frame TRAC Knock-In as a Dual-Editing Strategy

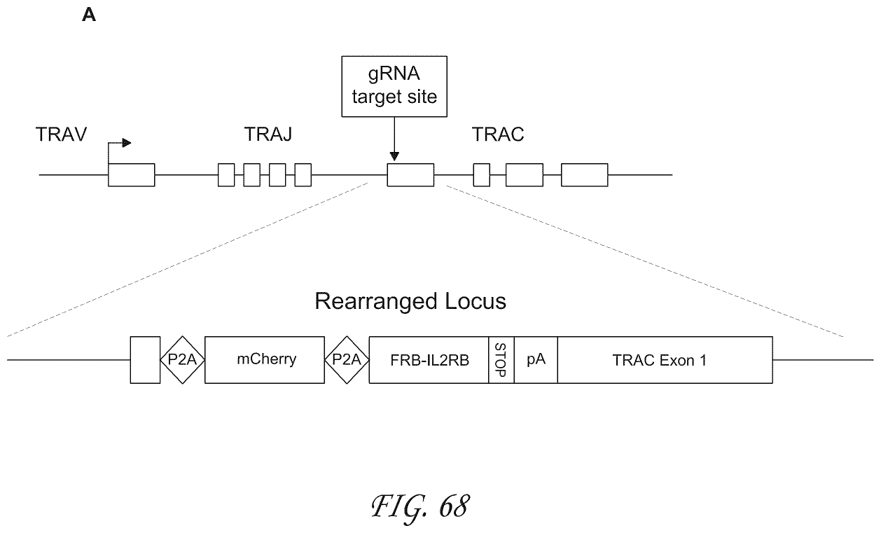

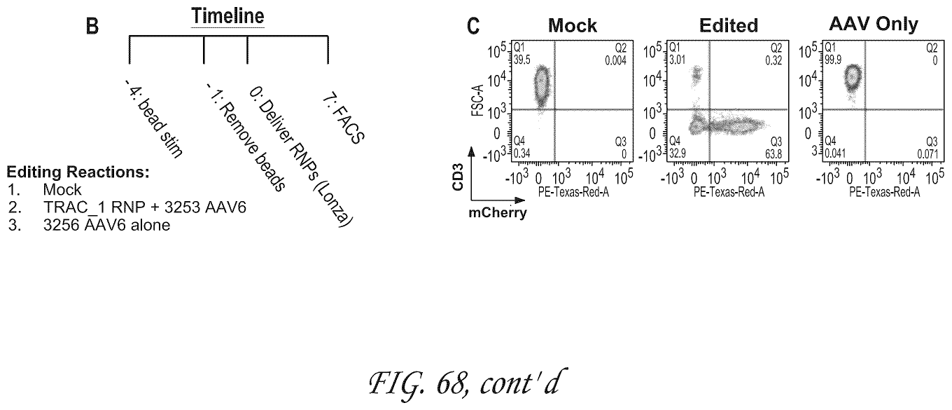

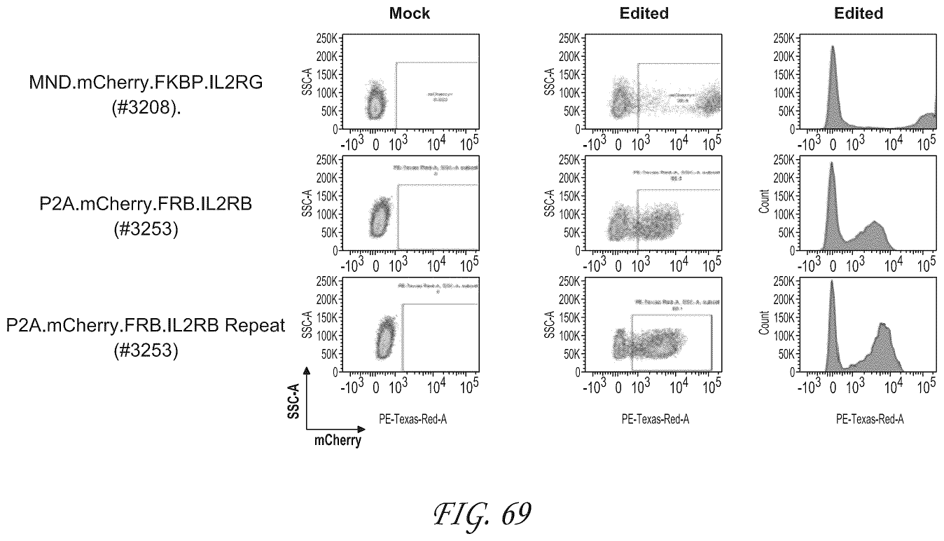

As an additional modification/improvement in our dual-editing strategies, methods were established for in-frame knock-in of a promoter-less TCR cassette including components of the IL-2 CISC, by targeting the first exon of the TRAC locus (FIG. 67). This editing strategy drives expression of the antigen-specific TCR via the promoter/enhancer activity of the endogenous TRAC locus. Advantages of this approach include elimination of endogenous TCR expression (and the potential for improper pairing with delivered TCR components) and concomitant near-endogenous levels of autoantigen-specific TCR expression. To establish this approach, gene editing and exogenous gene expression were tested with a proof-of-concept mCherry IL-2 CISC-containing construct (#3253) (FIG. 68). 63.8% of cells were mCherry-positive with concomitant loss of CD3 (down from 99.9% in AAV-only to 3.01% in P2A.mCherry.FRB.IL2RB (#3253) edited cells). Following gene-editing, mCherry expression was easily detected by flow cytometry. As expected, the MFI of mCherry expression in P2A.mCherry.FRB.IL2RB (#3253) edited cells was lower compared to mCherry expression in T cells edited in the same locus using MND promoter expression cassette (MND.mCherry.FKBP.IL2RG (#3208) FIG. 69). Thus, this HDR approach successfully permitted transgene expression via the promoter/enhancer activity of the endogenous TRAC locus.

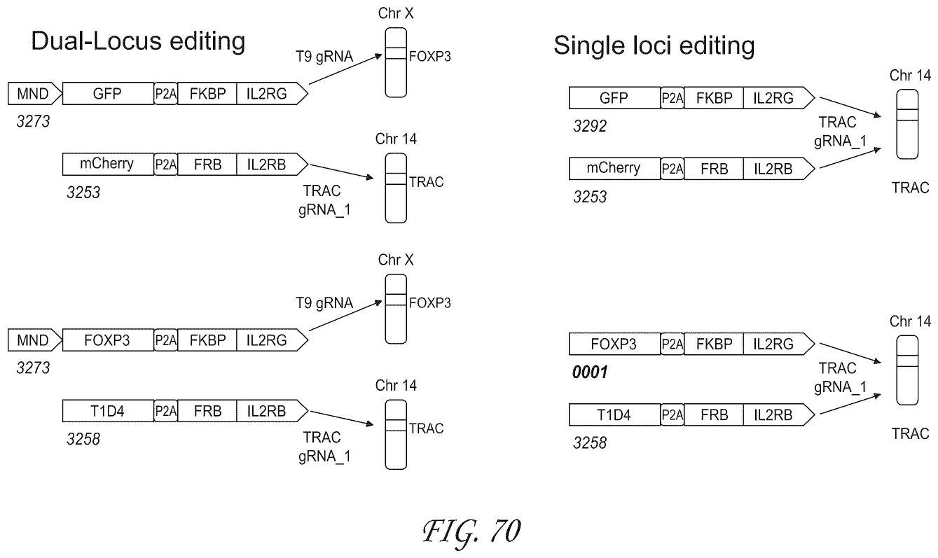

In follow-up studies, two distinct HDR donor cassettes are used to achieve dual-editing of TRAC (and capture of the endogenous promoter) and/or editing of both the TRAC and FOXP3 loci. HDR donors introduce split components of the IL-2 CISC to permit enrichment of dual-edited cells in parallel with delivery of the Ag-specific TCR (under TRAC promoter) and FOXP3 expression via cDNA expression or via locking on expression of endogenous FOXP3. Two-loci dual editing is tested using mCherry Split CISC with endogenous TRAC promoter (P2A.mCherry.FRB.IL2RB (#3253)) paired with MND.GFP.FKBP.IL2RG (#3273) for editing into the FOXP3 locus. Expression of the two components of the IL-2 split-CISC from two distinct promoters may affect overall CISC function, so single-locus dual editing is also tested using P2A.mCherry.FRB.IL2RB (#3253) and P2A.GFP.FKBP.IL2RG (#3292) to drive both components of the IL-2 CISC off the endogenous TRAC promoter (FIG. 70).

Following successful bi-allelic editing (using one or both promoter-less mCherry/GFP constructs and enrichment of dual edited cells using Rapalog), constructs are generated that can be used to introduce FOXP3 and antigen-specific TCR (T1D4) in combination with the IL-2 CISC components as an alternative approach to generate antigen-specific FOXP3+ airT cells. (See FIG. 70). airT generated using these strategies are compared to cells wherein the antigen-specific TCR is driven by the exogenous MND promoter.

### Dual Editing Using Decoy-CISC (Split-DISC) Constructs

Although CISC-expressing cells expand efficiently in the presence of a heterodimerizing Rapalog (AP21967), the FDA-approved drug, Rapamycin, may be preferred for clinical application. Intracellular binding of Rapamycin to its target mTOR has well-documented inhibitory effects on T-cell proliferation. Constructs and methods to address this problem by utilizing a naked “decoy” FRB domain expressed intracellularly in CISC-expressing cells are disclosed in WO2019210057, which is incorporated by reference in its entirety. Constructs that express the naked FRB domain along with the CISC are designated as “decoy-CISC,” or “DISC.”

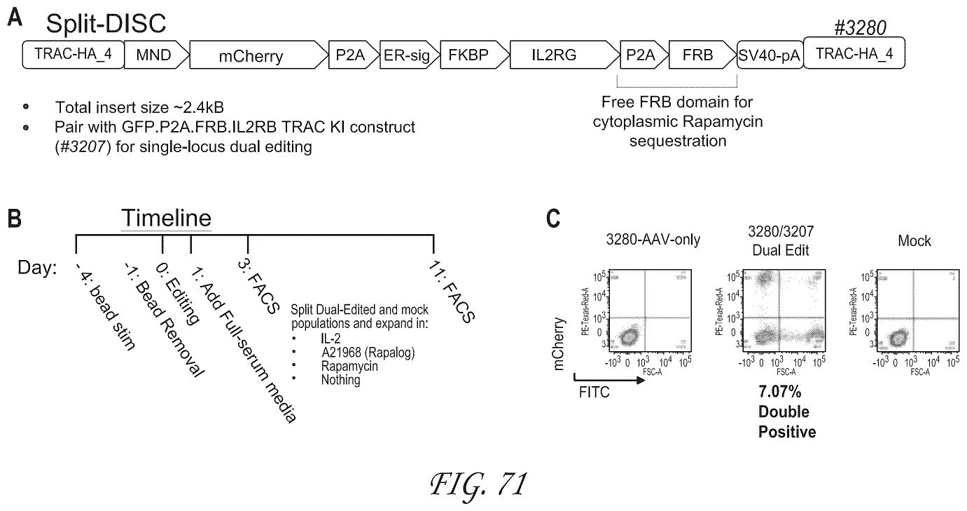

To determine if the DISC would work with dual editing approach, CISC containing constructs were modified to include an additional naked FRB domain located 3′ of the CISC receptor (FIG. 70, FIG. 71). Constructs with the additional FRB domain along with the CISC components are designated as “decoy-CISC or “DISC”. When utilized in a dual-editing approach, constructs are designated as split-DISC. Using the split-DISC, the naked FRB domain competed with the endogenous FRB domain of mTOR for binding to Rapamycin, thereby resulting in Rapamycin-mediated signaling through the split-CISC components without the associated inhibition of cell growth.

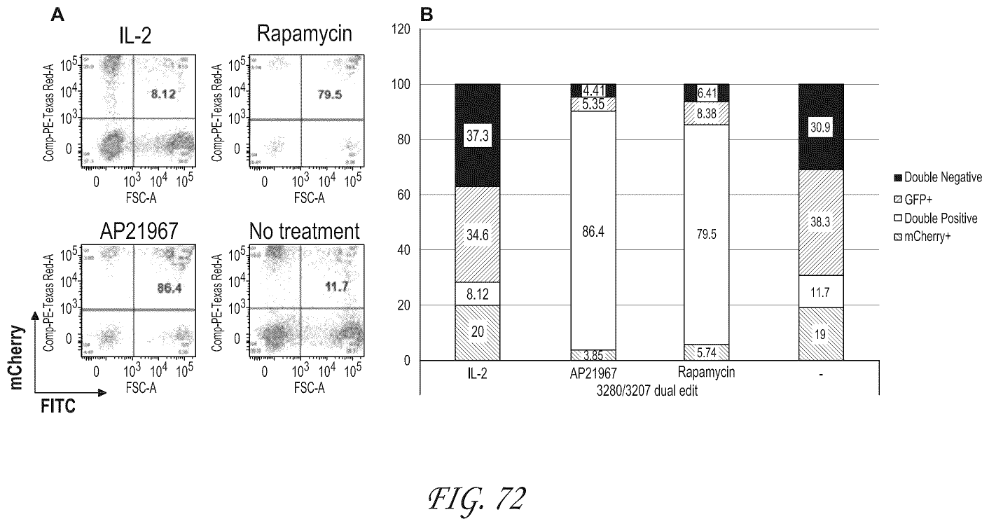

As shown in FIG. 71, T cells dual-edited with the mCherry CISC construct containing the added FRB domain (mCherry DISC, #3280) and the GFP CISC construct (#3207), expressed 7.07% double-positive GFP/mCherry cells. As predicted with the DISC construct, the double-positive GFP/mCherry cells enriched to a similar level following treatment with either Rapamycin or Rapalog (AP21967) (79.5% and 86.4% respectively) (FIG. 72).

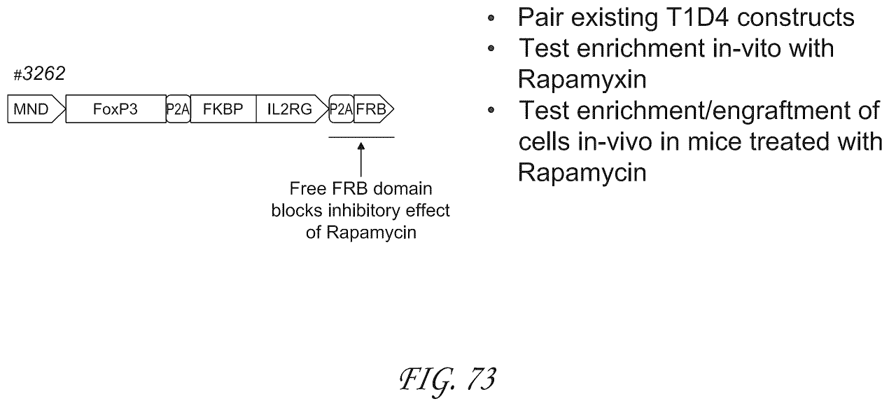

Experiments are performed to determine the in vivo enrichment/engraftment of GFP/mCherry-split-DISC edited cells in NSG mice treated with Rapamycin. Dual-edited GFP/mCherry cells exhibit increased engraftment/enrichment in vivo. These studies are expanded using FOXP3− and T1D4-containing constructs to evaluate engraftment and expansion of islet-specific airT in vivo. The DISC construct (MND.FOXP3.FKBP.IL2RG.FRB, #3262) shown in FIG. 73 is paired with existing #3243 MND.T1D4.FRB.IL2RB for dual-editing into the TRAC locus for development of islet-specific airT that can be enriched in vivo using Rapamycin.

### Example 11—In Vitro and In Vivo Functional Activities of Ag-Specific Murine airT In Vitro Characterization of Murine airT Products

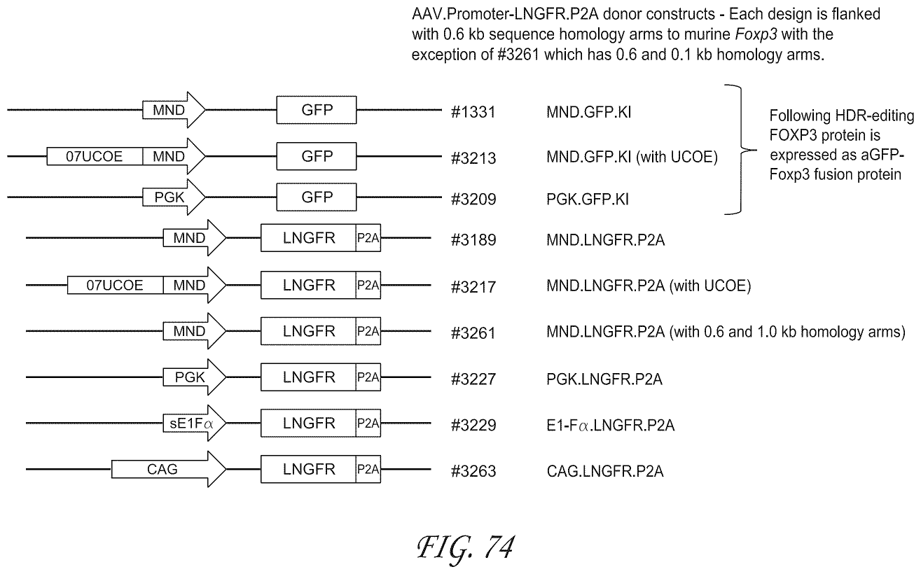

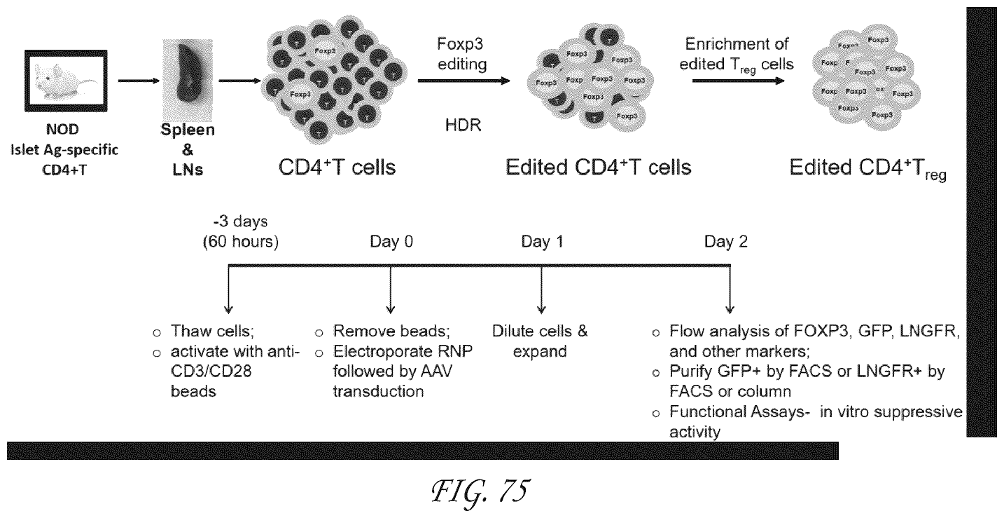

Studies designed to identify advantageous HDR donor template designs for generating airT products from murine CD4+ T cells with a suppressive Treg-like phenotype were performed. Questions addressed by these studies included: a) which of the tested promoter/enhancer constructs provided the best airT performance in vitro and, ultimately, in vivo and b) whether use of the clinically relevant cis-linked selectable marker LNGFR allowed for enrichment of airT in a manner suitable to GMP manufacturing. FIG. 74 to FIG. 80 show results from experiments that (1) evaluated the use of the clinically relevant cis-linked selectable marker LNGFR as a method to enrich murine cells following editing; (2) tested a variety of candidate promoters (in addition to MND promoter); (3) tested two Foxp3 homology arms of different size in the donor templates; and (4) compared donor templates with and without the UCOE element (as a potential means to limit silencing of an introduced promoter within the Foxp3 locus).

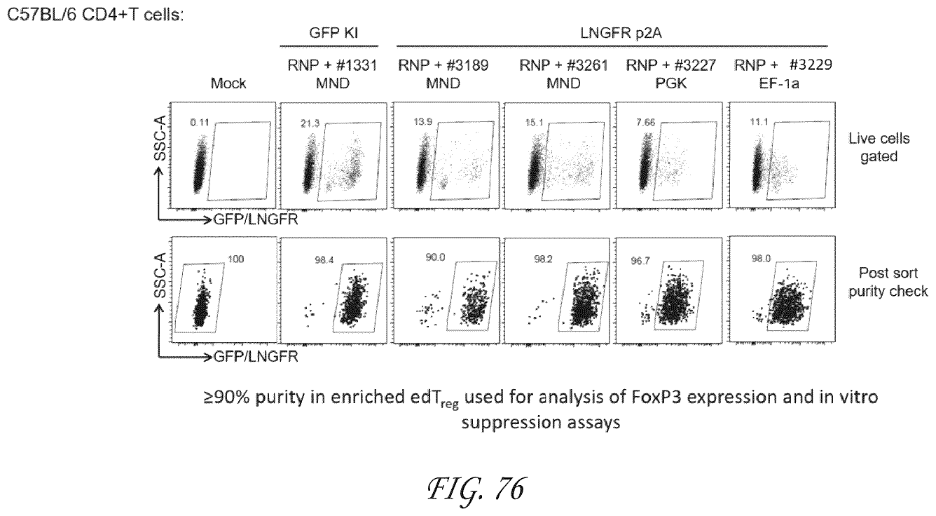

First, the effect of extending the homology arm of MND.LNGFR.P2A donor template from 0.6 kb to 1.0 kb of the Foxp3 gene on editing efficiency in C57BL/6 mouse CD4+ T cells was evaluated (FIG. 76). These studies indicate that MND.LNGFR.P2A #3261, encompassing a 1.0 kb arm, has a slightly higher editing efficiency compared to MND.LNGFR.P2A #3189, which contains a 0.6 kb arm. The improvement is ˜10%, and the increase in editing efficiency was reproducible between experiments, which enabled selection of AAV #3261 as a desirable targeted donor template for MND.LNGFR.P2A airT murine cells in the remaining studies. Also tested was the editing efficiency and purity following enrichment of C57BL/6 edTreg using AAV donor templates with alternative promoters (FIG. 76). This demonstrated that the overall editing and purity of LNGFR+ enriched cells were similar between AAV donor templates containing MND, PGK and EF-1a promoters. In addition, the purity following enrichment of LNGFR+ cells and GFP+ cells were comparable, further demonstrating that LNGFR can be used as a selection and enrichment method of murine CD4+ T cells.

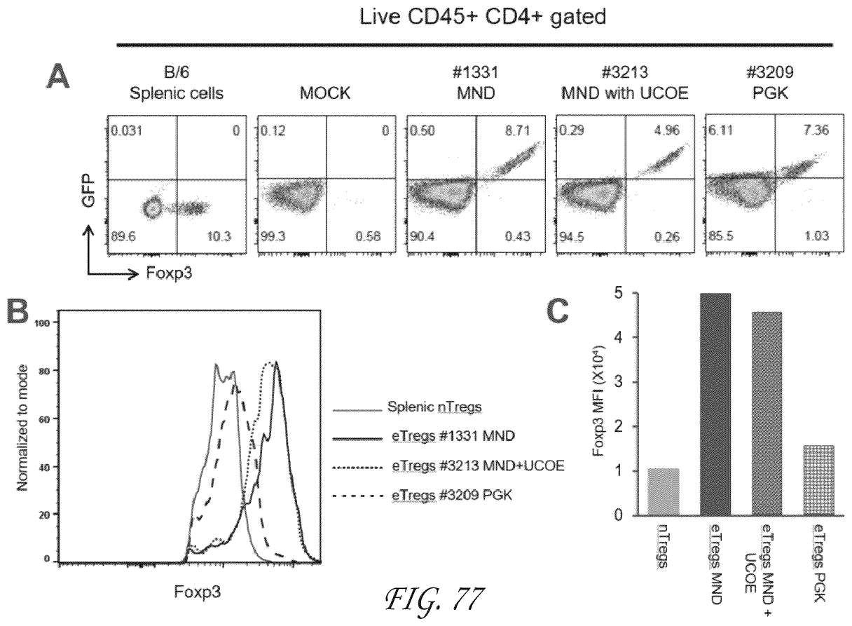

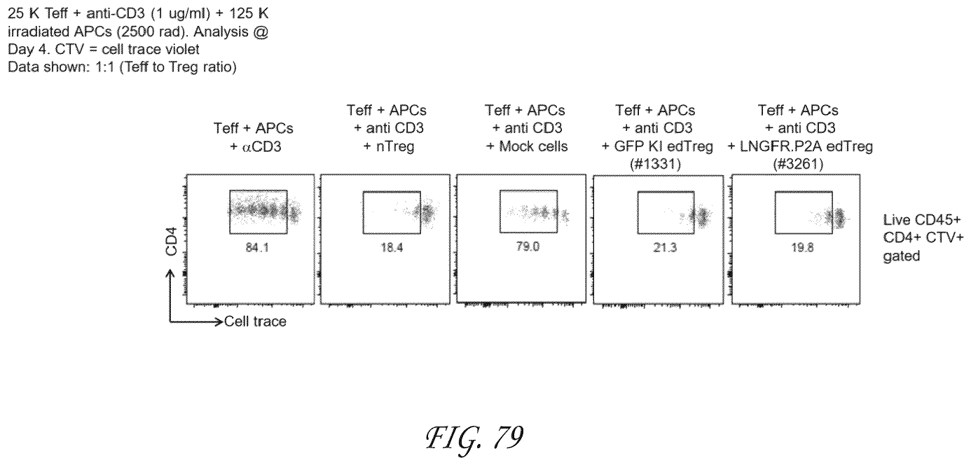

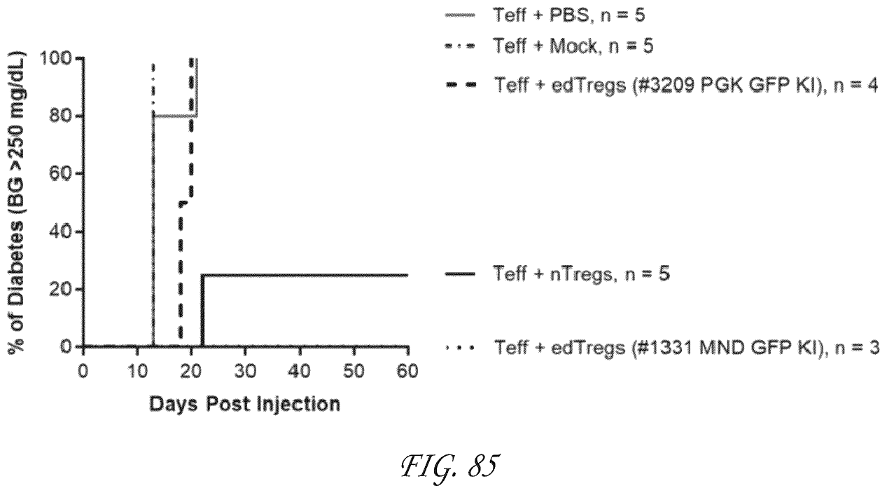

Although the editing efficiency and purity of edited cells using AAV donor templates with different promoters was similar, importantly, the level of FOXP3 expression varied depending the promoter. FIG. 77 shows evaluation of GFP and FOXP3 expression in Mock-edited, MND.GFP.KI- (#1331) and PGK.GFP.KI- (#3209) in C57BL/6-edited CD4+ T cells. The results showed that FOXP3 MFI was significantly higher in cells edited using MND.GFP.KI (#1331) compared to PGK.GFP.KI (#3209). The level of FOXP3 expression in PGK.GFP.KI- (#3209) edited CD4+ cells was similar to FOXP3 expression observed in splenic nTregs. These studies demonstrated the ability to introduce alternative promoters into the endogenous Foxp3 locus to control the overall level of FOXP3 in airT products. This may provide flexibility in product generation and may lead to airT with different functional properties. The relationship between FOXP3 expression level and in vitro and in vivo function was further explored in studies described in FIG. 79 and FIG. 85 (see below). The ability of Ubiquitous Chromatin Opening Element (UCOE) to stabilize FOXP3 expression was also tested (FIG. 77). This element can function to reduce silencing and limit potential negative impacts of promoter elements. These studies showed that FOXP3 was stable with or without UCOE and that inclusion of the UCOE element did not negatively impact the relative FOXP3 expression level (MND.GFP.KI #1331 compared with MND.GFP.KI with UCOE #3213), suggesting that UCOE shielded donor works effectively and inclusion of this element may be useful in airT products as it might protect expression in vivo or over time, providing improving duration of functional activity.

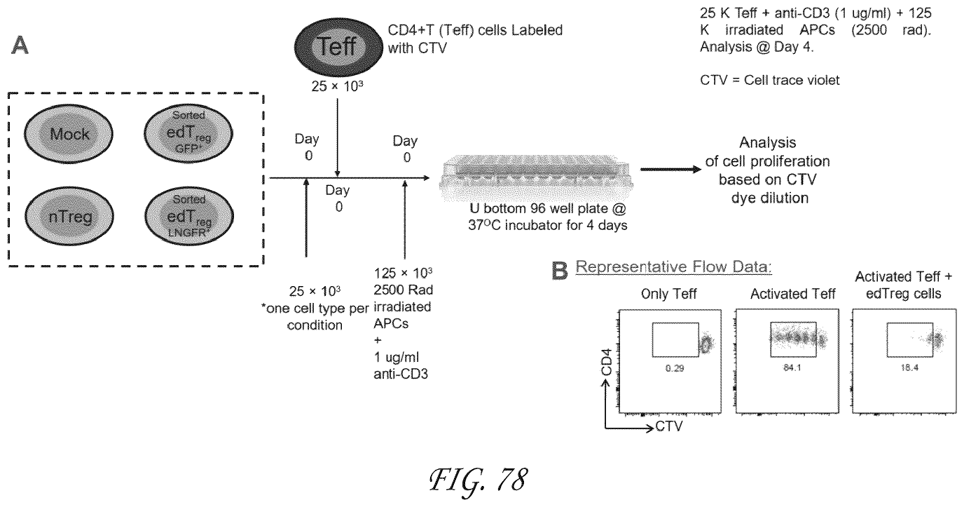

Additional studies were performed to assess: (a) the functional activity of LNGFR− expressing airT cells in vitro; and (b) determine if the promoter driving endogenous FOXP3 expression manifested any impact on Treg functional activity. An in vitro suppression assay outlined in FIG. 78 was utilized to analyze T_{eff} cell proliferation in the presence and absence of sorted MND.GFP.KI- and MND.LNGFR.P2A- airTs; these effects were compared to the activity of purified murine nTreg. The results shown in FIG. 79 demonstrated that murine airT (generated with the MND.GFP.KI or MND.LNGFR.P2A HDR donors) and nTregs exhibited comparable, robust in vitro suppressive function. These findings also demonstrated that airT cells expressing the LNGFR selectable marker (MND.LNGFR.P2A #3261) can be successfully used as a selection and enhancement method of murine CD4+ cells without a loss in functional activity and are useful for modeling in vitro and in vivo functional activity of human airT products that utilize the same clinically relevant selection marker.

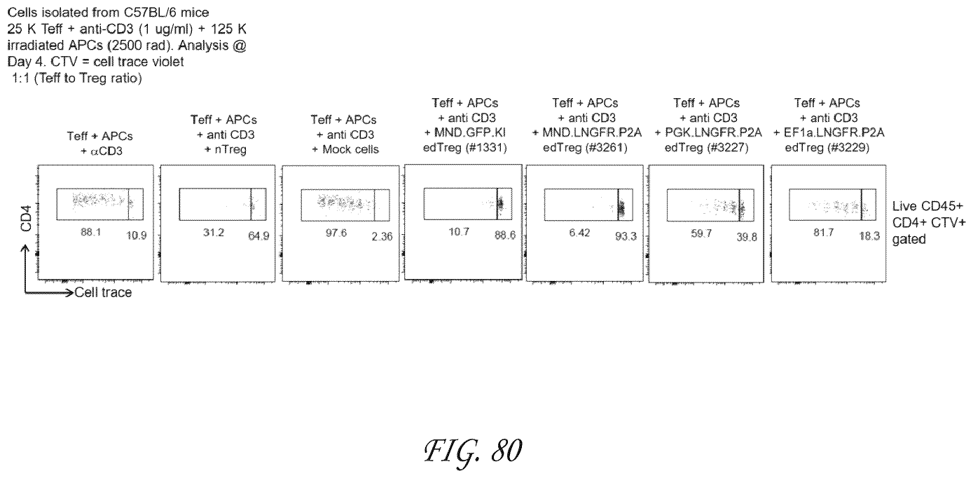

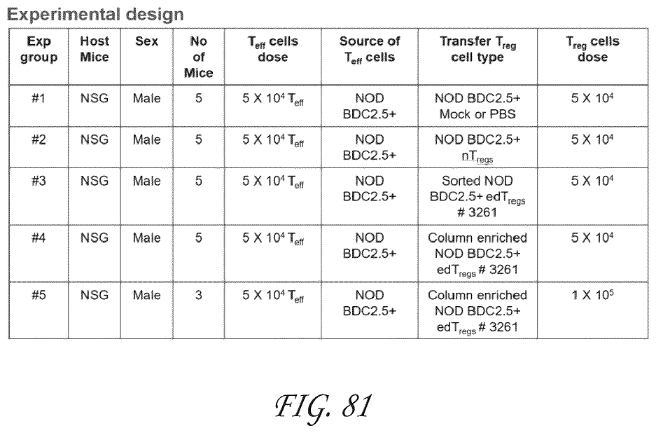

Using the same in vitro suppression assay, the functional activity of different strength promoters was explored to determine if FOXP3 expression levels (evaluated in FIG. 77) correlated with functional activity. FIG. 80 shows a comparison of LNGFR constructs utilizing the MND, PGK and EF-1a promoters (MND.LNGFR.P2A, PGK.LNGFR.P2A and EF-1a.LNGFR.P2A respectively) with MND.GFP.KI C57BL/6 edited CD4+ T cells and nTreg. The results showed that murine airT with MND promoter exhibited suppressive function that was comparable to nTreg. In contrast to the MND promoter constructs, airT cells utilizing the PGK promoter exhibited only partial in vitro suppressive function and airT utilizing the EF-1a promoter failed to suppress. Interestingly, although the PGK.GFP.KI-edited cells expressed FOXP3 levels similar to nTregs (FIG. 77), the in vitro suppressive activity of nTreg was significantly greater than PGK.LNGFR.P2A edited cells. These findings suggested the surprising result that a threshold level of FOXP3 expression in edited CD4+ T cells may be necessary to provide proper reprogramming and effective in vitro functional activity. The results described below also clearly demonstrate that the MND promoter was effective for reducing diabetes in vivo.

### In Vivo Functional Characterization of edTreg Products

The experiments summarized above suggest that murine airT cells containing a clinically relevant cis-linked LNGFR selectable marker retained functional activity in vitro and that the MND promotor had superior in vitro suppressive activity compared to alternative promoters. These studies were expanded to evaluate islet-specific airT product in an NSG adoptive transfer diabetes model where transfer of islet-specific NOD (murine) CD4+T cells into adult recipient NSG mice triggers rapid onset of diabetes.

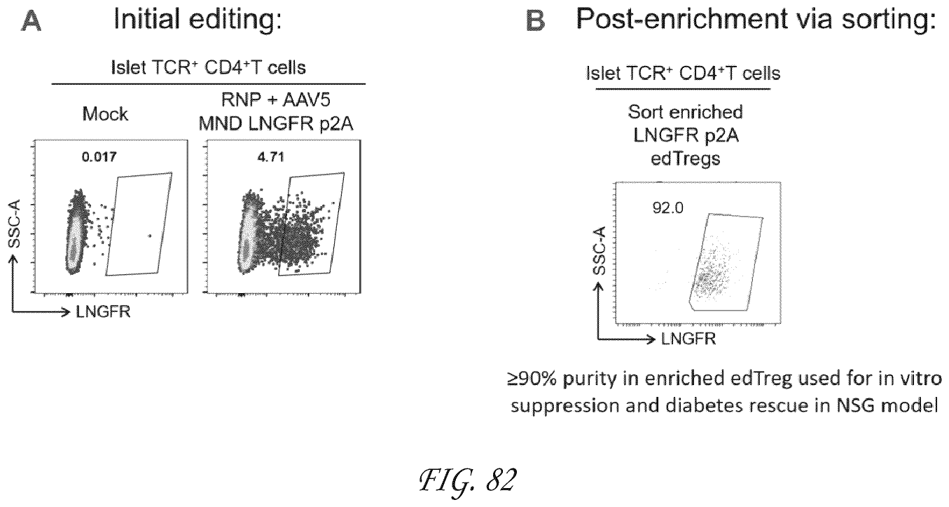

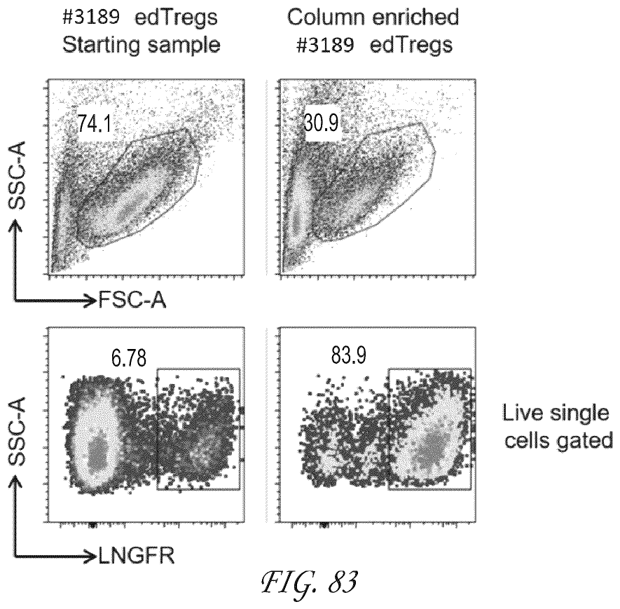

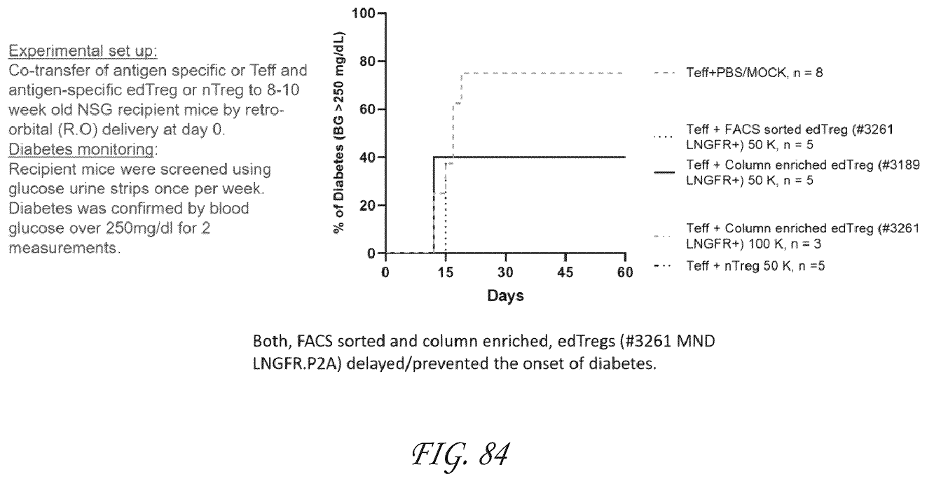

Using this model, islet-specific MND.LNGFR.P2A airT derived from NOD BDC2.5 mice were evaluated for the ability to delay or prevent diabetes development. Although the in vitro experiments described in FIGS. 76-80 utilize cells enriched via cell sorting, the sorting process is both time-consuming and costly. In addition, sorting may well impact the engraftment and/or survival of cells post-adoptive transfer in vivo. To enable efficient enrichment of murine gene-edited airT that can be used in vivo, the purification and functional activity of airT purified using alternative methods was compared: (1) LNGFR+ cell enrichment through cell sorting by flow cytometer and (2) LNGFR+ cell enrichment using LNGFR column separation. FIGS. 82-83 show the flow plots prior to and post-purification using sorting and column enrichment. Although purification using the sorting method yielded an enriched population of LNGFR+ cells, the column enrichment produced ˜84% pure cell population with a significant savings in time and resources. Importantly, both the column-enriched LNGFR+ airT and sorted cells delayed or prevented the onset of diabetes (FIG. 84).

Together, the data in FIG. 82 to FIG. 84 demonstrated the capacity to generate highly purified islet-specific edited cells using LNGFR column separation and by FACS sorting. Both products expressed high levels of LNGFR/FOXP3 and showed Treg-like phenotypes by reducing or preventing diabetes in vivo.

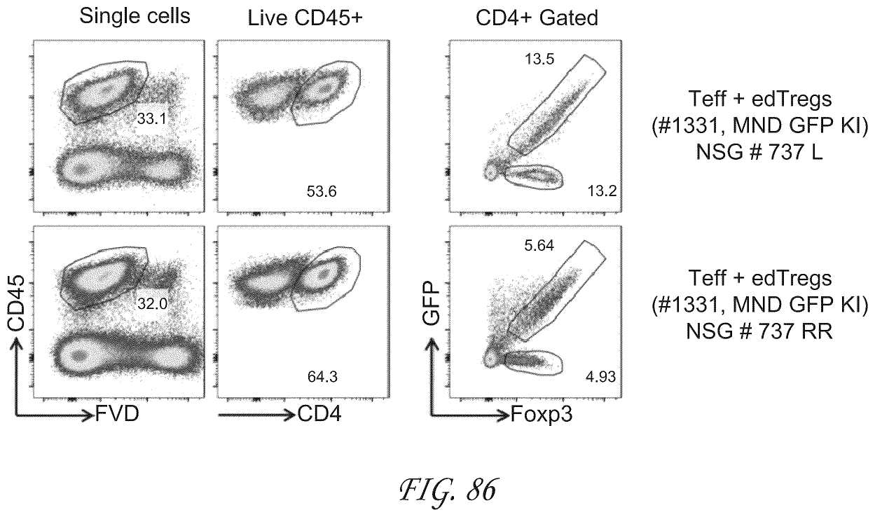

Finally, FIG. 85 shows that functional activity of islet-specific airT products in the NSG adoptive transfer model varied depending on the promoter used to drive endogenous FOXP3. Cells edited with MND.GFP.KI and nTregs both delayed the onset or prevented diabetes, but PGK.GFP.KI airT did not. This result was consistent with the in vitro suppression data shown in FIG. 80 suggesting that selection of the promoter played a role in optimal function. Importantly, consistent with protection from diabetes, islet-specific airT cells homed to the pancreas and persisted in the NSG model with stable FOXP3 expression (FIG. 86).

These data demonstrated the capacity to engineer mouse airT from T_{eff} cells for in vitro and in vivo studies. Consistent with the findings in human T cells, the MND promoter effectively converted mouse T_{eff} into airT cells with high levels of FOXP3 expression and robust in vitro suppressive activity comparable with nTreg. Importantly, murine islet-specific MND airT and nTreg: (1) exhibited comparable, robust in vitro suppressive function; (2) blocked diabetes triggered by islet-specific T_{eff} in recipient mice. Moreover, the data showed that (3) airT cells expressing the LNGFR selectable marker can be enriched in vitro without a loss in functional activity and can function in vivo and (4) MND airT outperformed airT generated with alternative promoters, including PGK and EF1A, demonstrating that choice of the promoter played a role in improved function.

The NSG adoptive transfer diabetes model described permitted rapid assessment of key functional features of murine airT including: LN trafficking, expansion, activation status, and the capacity to limit initial T_{eff} activation. These approaches were used to compare the functional activity of antigen-specific, LNGFR enriched airT in an immunocompetent NOD mouse model of T1D.

### Editing at the Rosa26 Locus for Generating Murine T Cells Edited Cells

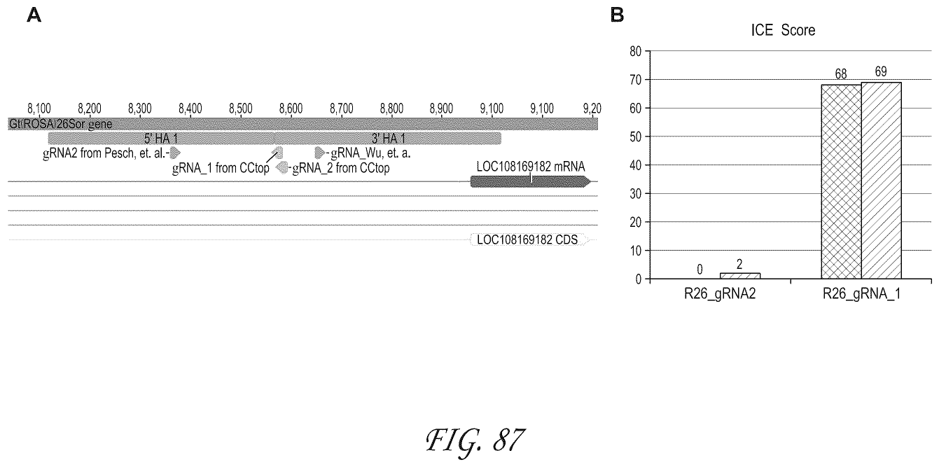

To expand the tool set for assessing the efficacy of Ag-specific FOXP3 airT in animal models of diabetes or other autoimmune conditions, gRNA targeting the murine Rosa26 locus were designed and tested. This well-characterized safe harbor locus has historically been used for stable expression of integrated transgenes in mouse models. Two novel gRNA target sequences within an intronic region of Rosa26, proximal to published gRNA target sites, were selected and on-target site-specific activity measured by ICE (Inference of CRISPR Edits) after RNP delivery to primary mouse CD4+ T-cells. ICE confirmed specific indel induction for R26_gRNA_1 in Rosa26 (FIG. 87).

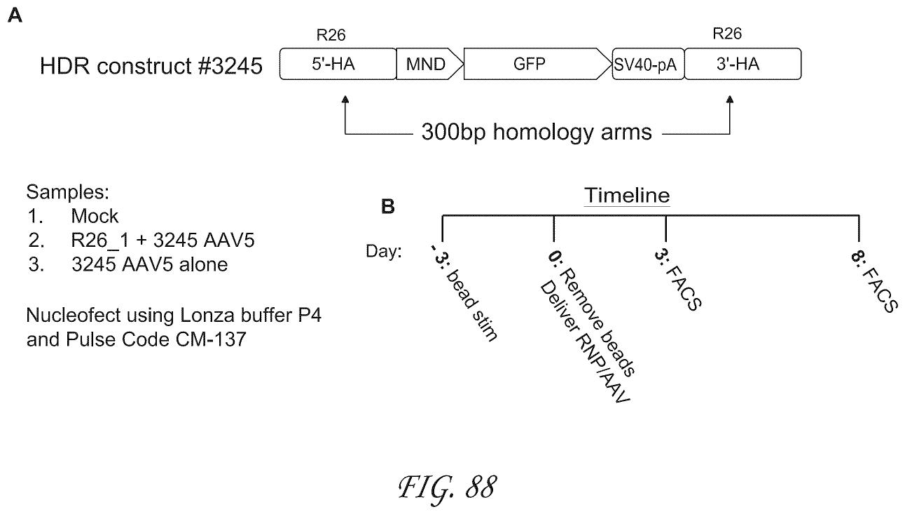

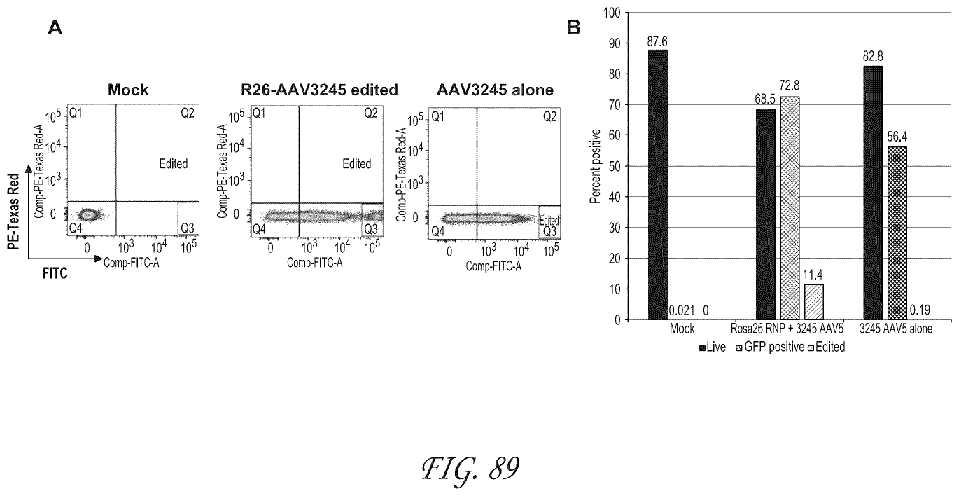

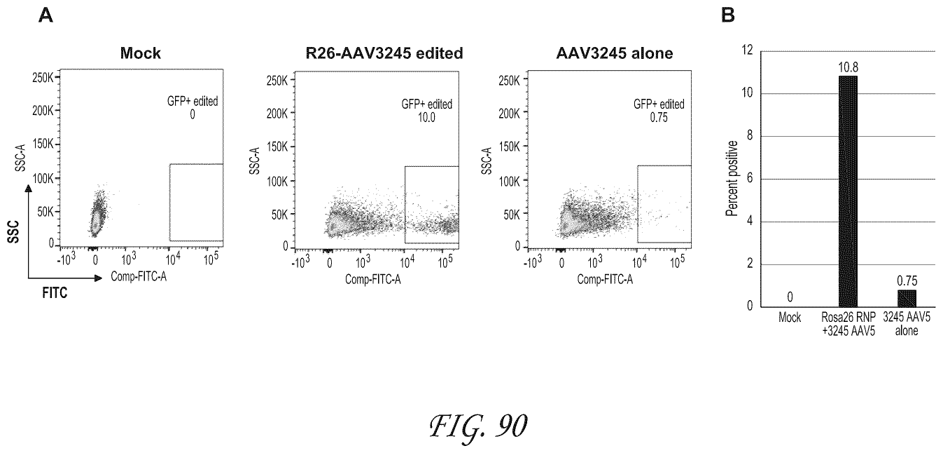

Next, the ability to edit murine T cells using constructs that would allow easy tracking of successfully edited cells was tested. A MND-GFP cassette was generated flanked by identical 300 base pair Rosa26 homology arms matched to R26_gRNA_1 (#3245) and was used to generate Rosa26 edited T cells with stable expression of GFP. The timeline for cell expansion, editing and analysis is shown in FIG. 88. The resulting FACS analysis demonstrates 11.4% GFP high cells with AAV #3245 plus RNP compared to 0.02% with AAV #3245 alone 3 days post-edit, confirming successful integration of MND-GFP repair cassette into the Rosa26 locus (FIG. 89). FACS analysis carried at 8 days post-editing showed a similar percent of GFP+ cells (10.8%), indicating that the GFP expression is stable (FIG. 90).

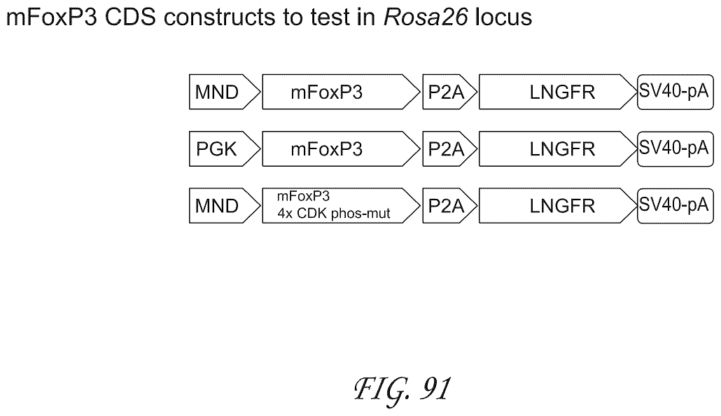

Having achieved editing murine T cells at the Rosa26 locus using the MND-GFP cassette, repair templates containing mFoxp3 CDS with LNGFR marker (for purification) and alternative candidate promoters (in addition to MND promoter) are developed to generate further constructs with stable expression of FOXP3 in this safe harbor locus in mouse cells (FIG. 91). These constructs are used to explore dual editing in mouse cells for the generation of murine antigen-specific FOXP3-expressing airT for use in mouse autoimmune models. Mutant FOXP3 variants are tested that are predicted to have increased stability. This includes the 4× CDK phosphorylation mutant, where a set of 4 target residues for cyclin-dependent kinase phosphorylation have been replaced with alanine, blocking phosphorylation events that have been linked to protein degradation.

These data demonstrate that the Rosa26 safe harbor locus can be used for HDR editing in mouse T cells. This advance permitted dual-editing studies of mouse T cells paralleling work in human T cells, facilitating nonclinical animal modeling of Ag-specific airT.

### Developing Tools for Expansion of Murine Cells Using CISC Elements

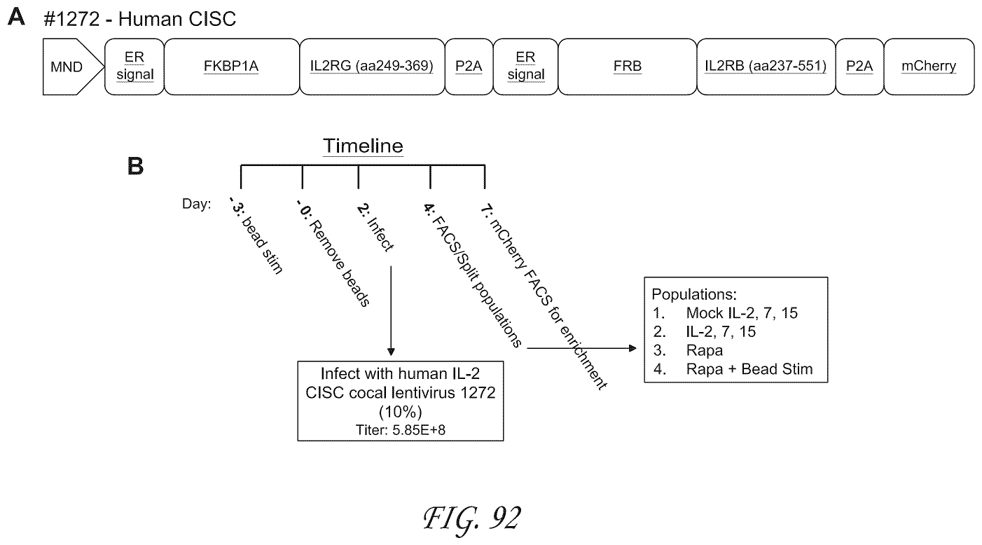

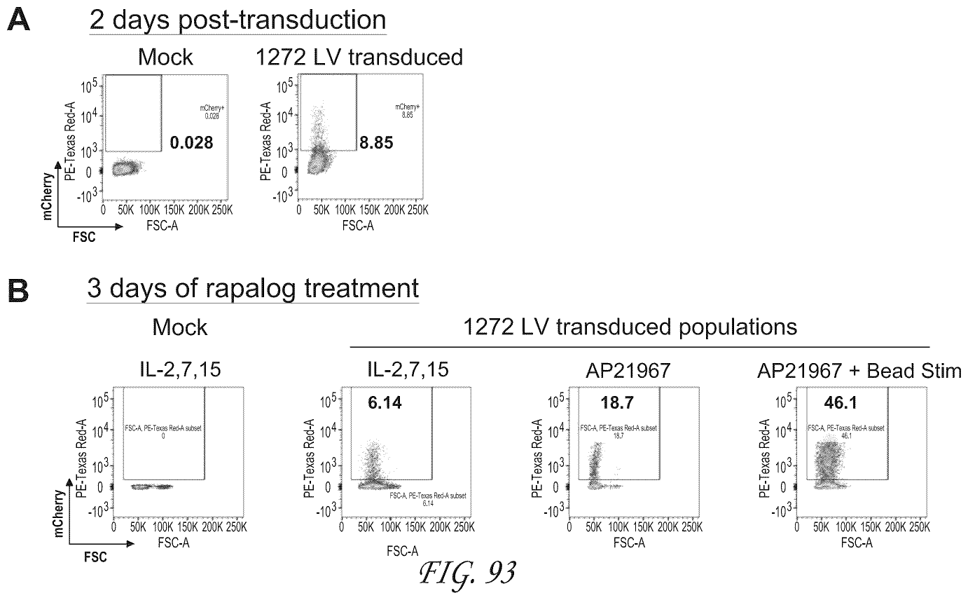

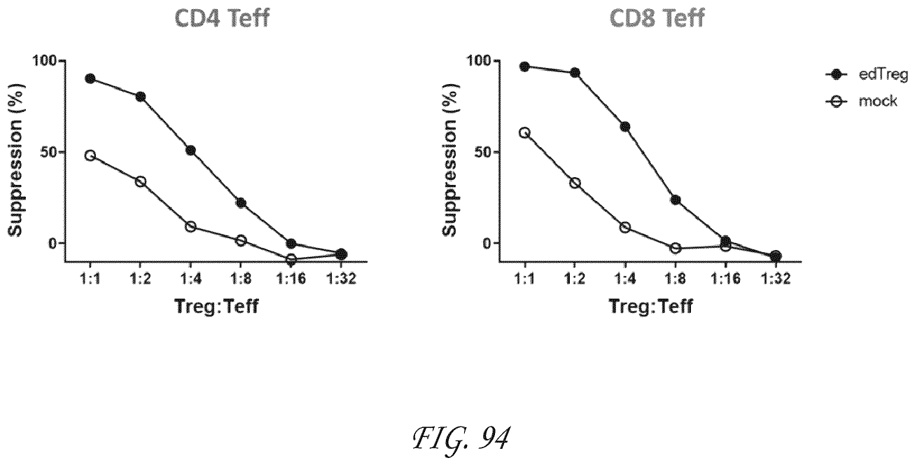

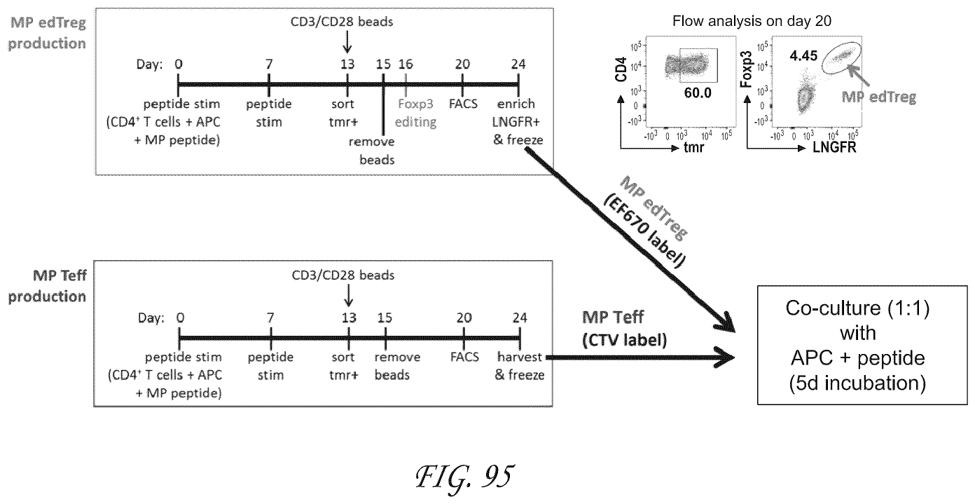

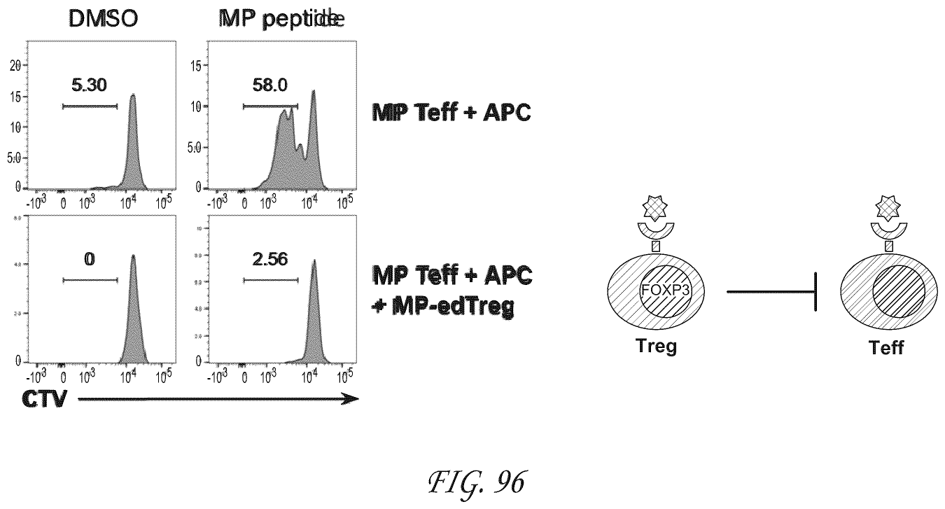

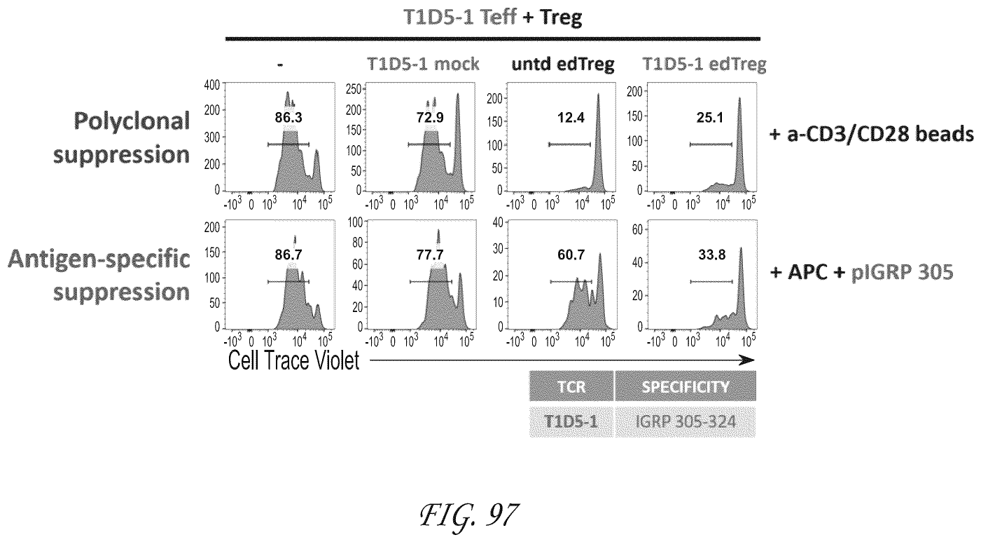

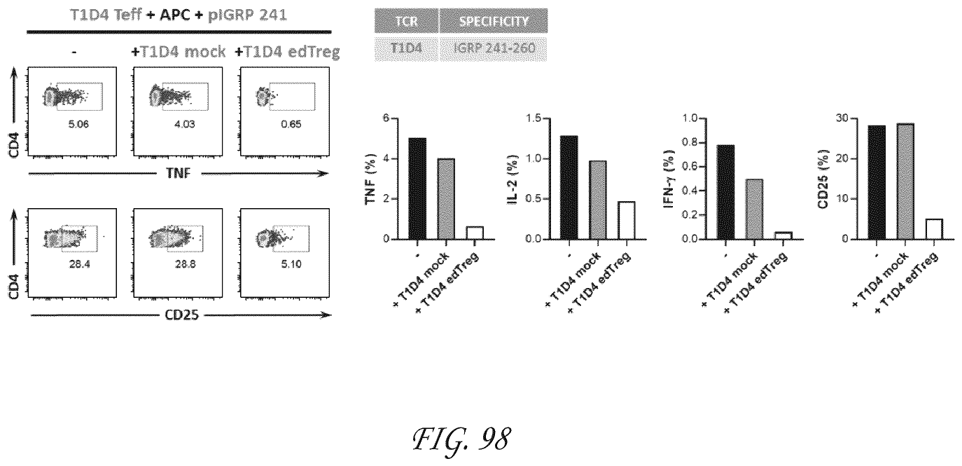

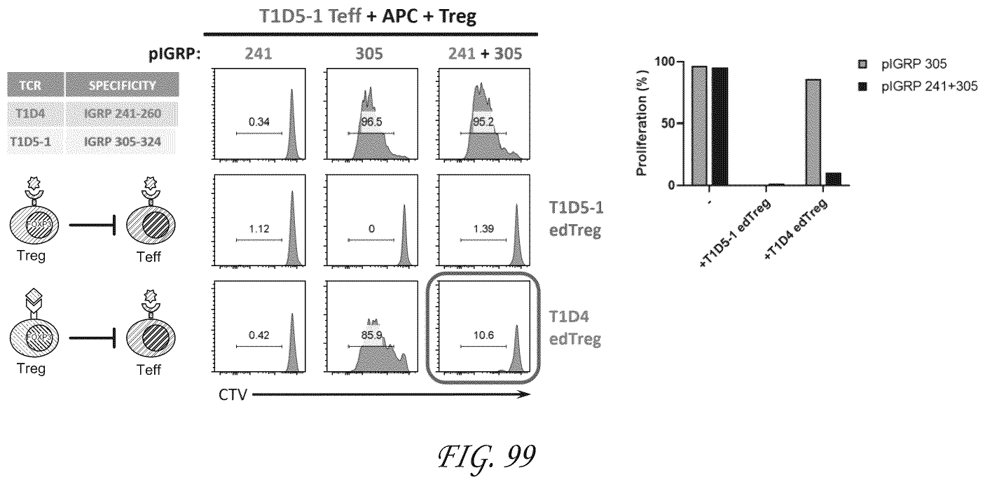

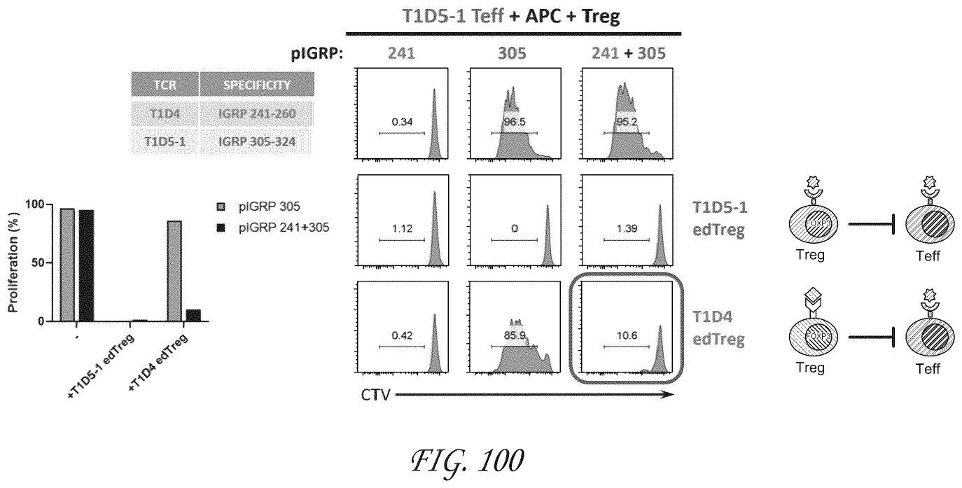

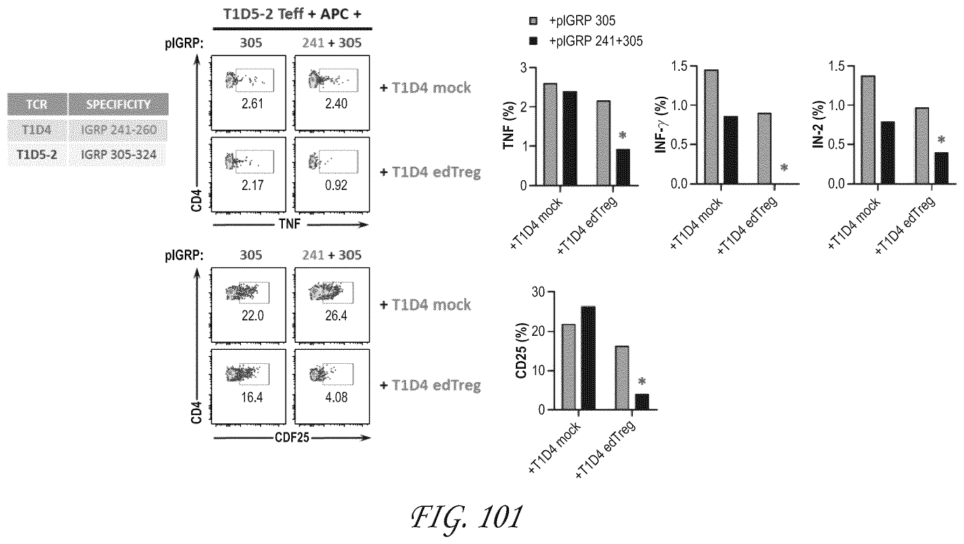

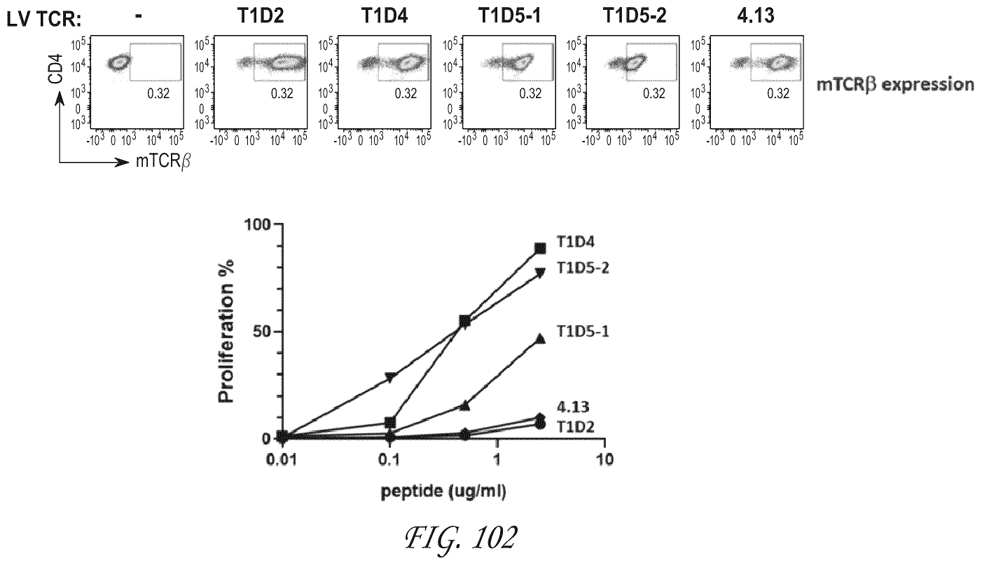

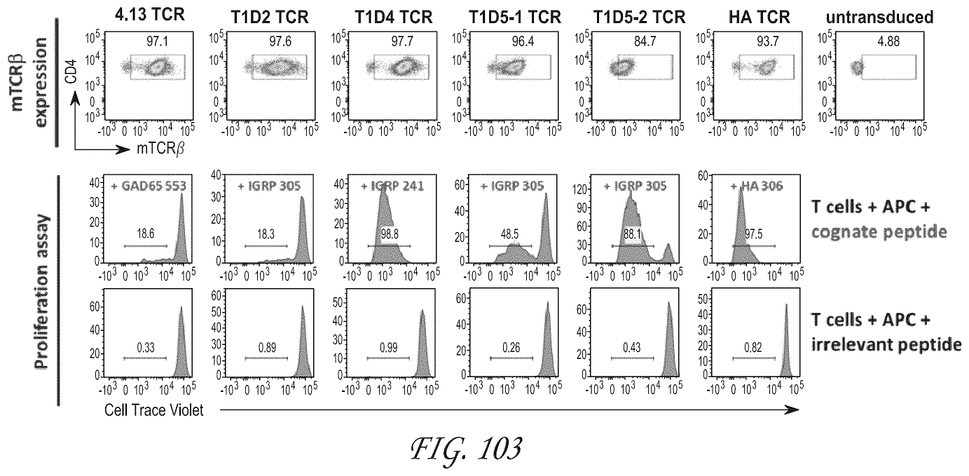

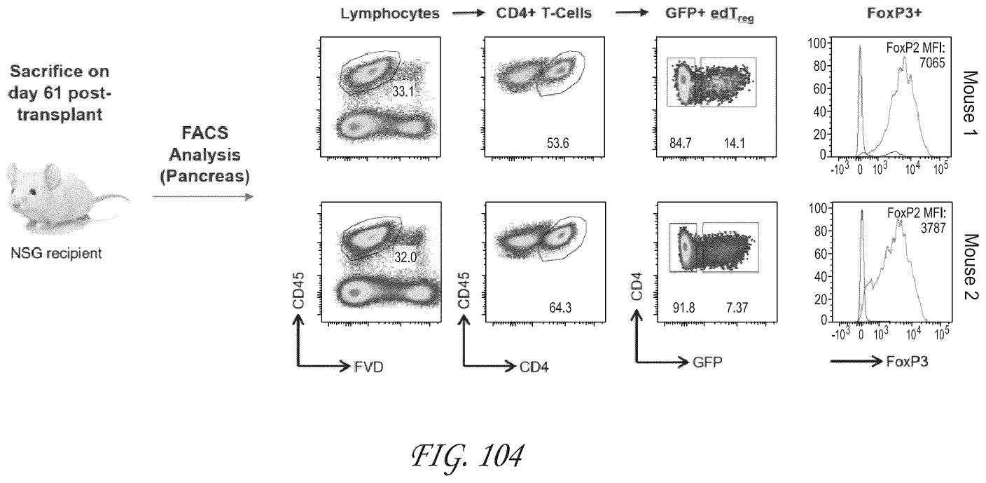

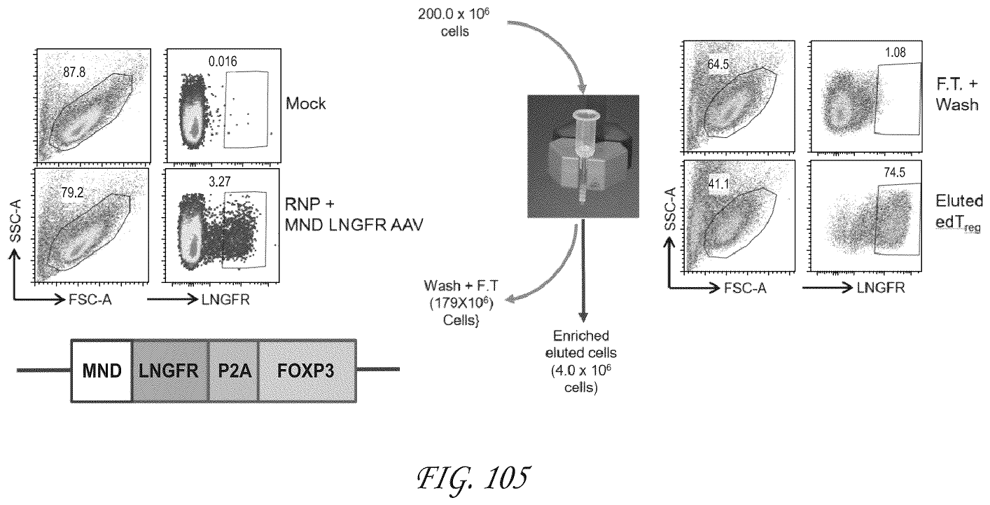

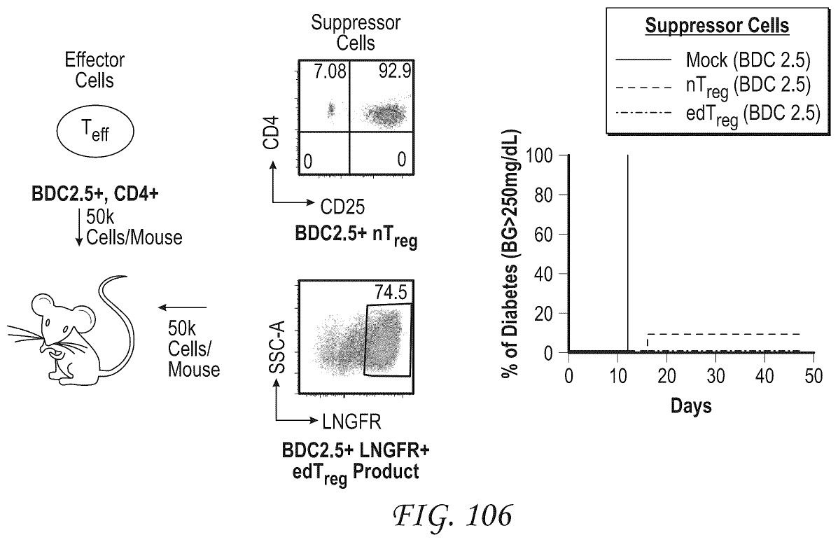

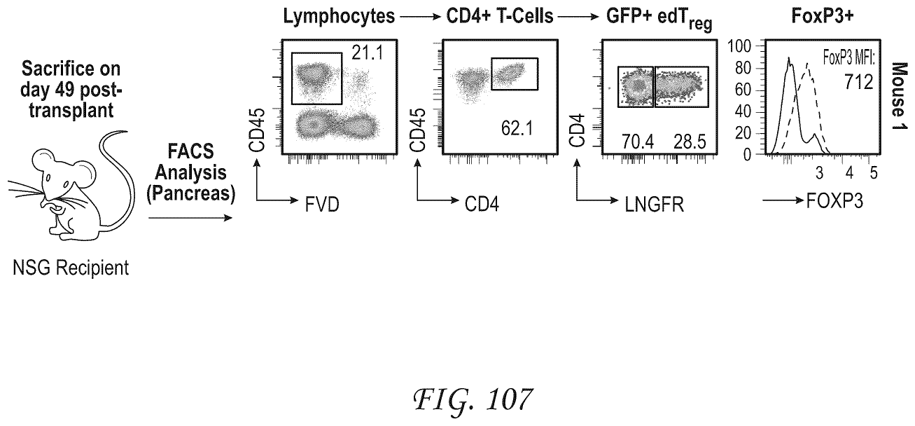

An important feature of a human antigen-specific airT platform is the potential to expand airT in vitro and in vivo using, as an example, the IL-2-CISC system. To assess the function of airT containing the IL-2-CISC cassette in immune competent animal disease models, experiments were performed to test whether the human IL-2R sequence containing CISC/DISC cassette can promote selective expansion of murine cells in vitro and in vivo with Rapalog/Rapamycin. These data demonstrated proof-of-concept using a lentiviral construct, #1272, that contained a MND promoter-driven mCherry reporter and cis-linked human IL-2 CISC elements. FIG. 92 shows the schematic of the lentiviral cassette and the timeline of T cell transduction, expansion and analysis. In this study, transduced cells were placed in either: (a) IL-2, IL-7 and IL-15; (b) Rapalog alone; or (c) Rapalog plus an additional CD3/CD28 bead stimulation 2 days after transduction. FIG. 93 demonstrates mCherry expression in 8.85% of the transduced cells and further enrichment after 3 days of Rapalog treatment. Enrichment was greatest (46.1%) when transduced T cells that were concurrently treated with both Rapalog and an additional CD3/CD28 bead stimulation.

These data show that murine CD4+ T cells can be enriched using the IL-2 CISC technology and that human CISC is functional in the mouse system. These findings demonstrate the feasibility of studies to examine enrichment and function of murine Ag-specific airT using the split-CISC/Split-DISC approach in non-clinical animal models, where one cassette is integrated into a first locus of the genome, and another cassette is integrated into a second, different locus of the genome.

### Example 12 Generation and Testing of Antigen-Specific edTreg

### RA Antigen-Specific TCRs Identified from RA Patients

RA antigen-specific TCRs were identified from T cells clones isolated from RA patients. Based on these sequences, lentiviral TCR constructs for TCR gene transfer were generated. TABLE 3 lists generated lentiviral constructs encoding RA antigen-specific TCRs, their epitope specificity, and HLA-restriction. Target T cell epitope sequences included citrulline modifications. TCRs recognizing citrullinated -vimentin, -aggrecan, -CILP, and enolase were identified from T cell clones that were previously isolated from RA patients

TABLE 3

| TCR | | | Target | SEQ ID | HLA- |
|---|---|---|---|---|---|
| name | Antigen | Epitope | sequence | NO | DR |
| Vim418 | vimentin | 418-431 | SSLNL | SEQ ID | *0404 |
| | | | (Cit)E | NO: 1408 | |
| | | | TNLDSL | | |
| Agg153 | aggrecan | 153-168 | IVFHY | SEQ ID | *0404 |
| | | | (Cit) | NO: 1409 | |
| | | | AIST | | |
| | | | (Cit) | | |
| | | | YTLDF | | |
| Agg520 | aggrecan | 520-539 | GYEQC | SEQ ID | *0401 |
| | | | DAGWL | NO: 1410 | |
| | | | (Cit) | | |
| | | | DQTV | | |
| | | | (Cit) | | |
| | | | YPIV | | |
| Agg553 | aggrecan | 553-570 | PGV(Cit) | SEQ ID | *0401 |
| | | | TYGV | NO: 1411 | |
| | | | (Cit) | | |
| | | | PSTET | | |
| | | | YDVY | | |
| Agg621 | aggrecan | 621-637 | KCYAG | SEQ ID | *0401 |
| | | | WLADG | NO: 1412 | |
| | | | SL(Cit) | | |
| | | | YPIV | | |
| CILP297- | CILP | 297-311 | ATIKA | SEQ ID | *0401 |
| 1 | | | EFV | NO: 1413 | |
| | | | (Cit) | | |
| | | | AETP | | |
| | | | YM | | |
| CILP297- | CILP | 297-311 | ATIKA | SEQ ID | *0401 |
| 2 | | | EFV | NO: 1414 | |
| | | | (Cit) | | |
| | | | AETPYM | | |
| Enol326 | enolase | 326-340 | K(Cit) | SEQ ID | *0401 |
| | | | IAKA | NO: 1415 | |
| | | | VNEK | | |
| | | | SCNCL | | |

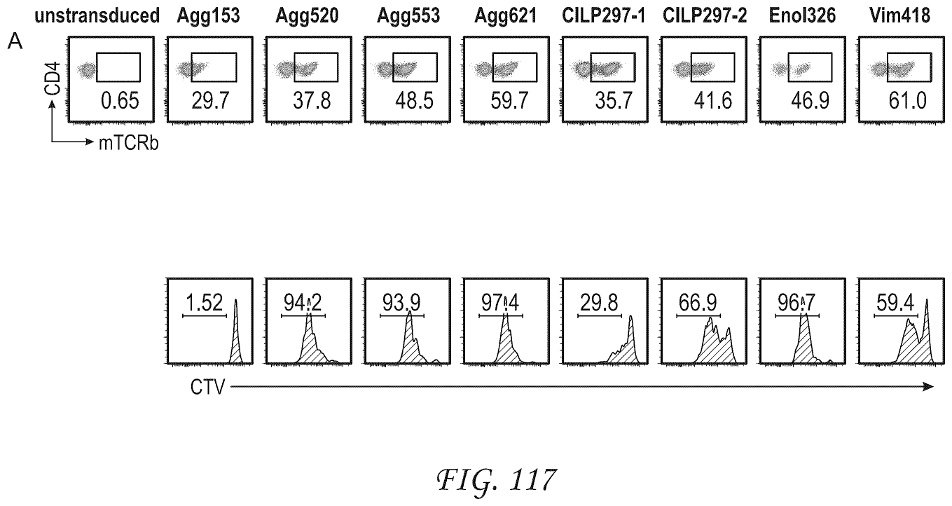

CD4+ T cells were isolated, activated with CD3/CD28 beads, and transduced with lentiviral RA Ag-specific TCRs. Flow plots show mTCRb expression gated on CD4+ cells day 9 post-transduction (FIG. 117A). CD4+ T cells transduced with RA Ag-specific TCRs were labeled with CTV and co-cultured with APC (irradiated PBMC) and their cognate peptide or DMSO for 3 days. Flow plots show cell proliferation as CTV dilution (FIG. 117B). RA-specific TCR expression was validated by T cell proliferation assays using peptides cognate with the TCRs and antigen presenting cells (APCs). T cells transduced with RA-specific TCRs (vimentin, aggrecan, CILP and enolase) proliferated in response to their cognate peptides and APC.

### Suppressive Activity of Enolase-Specific edTreg

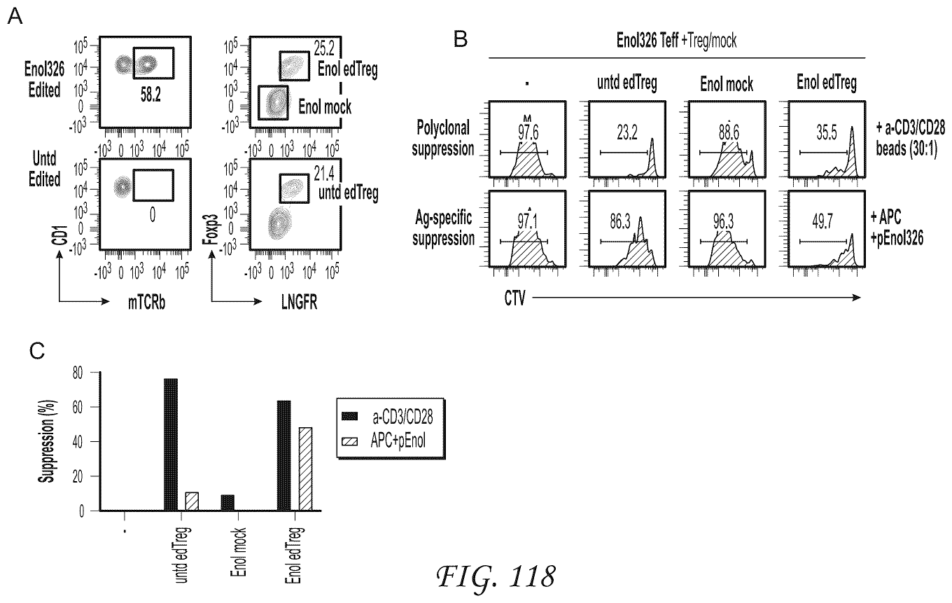

Antigen-specific Treg were generated by editing the Foxp3 locus in CD4 T cells that had been transduced with enolase TCRs. This resulted in the successful generation of enolase-specific edTreg. FIG. 118A depicts flow plots of mTCRb expression and LNGFR/Foxp3 expression in edited cells without LV transduction (Untd Edited) and edited cells expressing Enol326-TCR (Enol326 Edited) on day 7. edTreg cells were enriched by LNGFR expression on day 10 and LNGFR− cells were used as mock cells for suppression assays. The transduced Enol326-TCR had a specificity for an epitope of Enolase 326-340.

FIG. 118B depicts a polyclonal suppression assay and an antigen-specific suppressing assay using enolase-specific edTreg. Enolase-specific Teff cells were produced from LV Enol326-TCR transduction of CD4+ T cells and expanded for 15 days. For the polyclonal assay, Enol326 Teff were incubated with anti-CD3/CD28 beads at 1:30 of bead to cell ratio with no Treg, untd edTreg, Enol326 edTreg, or mock cells. For the antigen-specific suppression assay, Enol326 Teff cells were co-cultured with APCs and Enol326 peptide in the presence of no Treg, untransduced (untd) edTreg, Enol326 edTreg, or mock cells. For all the suppression assay set up, Teff and edTreg or mock cells were labeled with CTV and EF670, respectively and co-cultured at 1:1 ratio. 4 days after the co-culture, cells were stained and analyzed for Teff proliferation as dilution of CTV. FIG. 118C depicts a graph of percentage suppression of Teff proliferation by no Treg, untd edTreg, Enol edTreg, or mock in the presence of a-CD3/CD28 (black) or APC and enolase peptide (grey) calculated from percentage proliferation in FIG. 118B. The enolase-specific edTregs showed antigen-specific and polyclonal suppressive function of antigen-specific T effector cells by in vitro suppression assays.

### Suppressive Activity of CILP-Specific edTreg

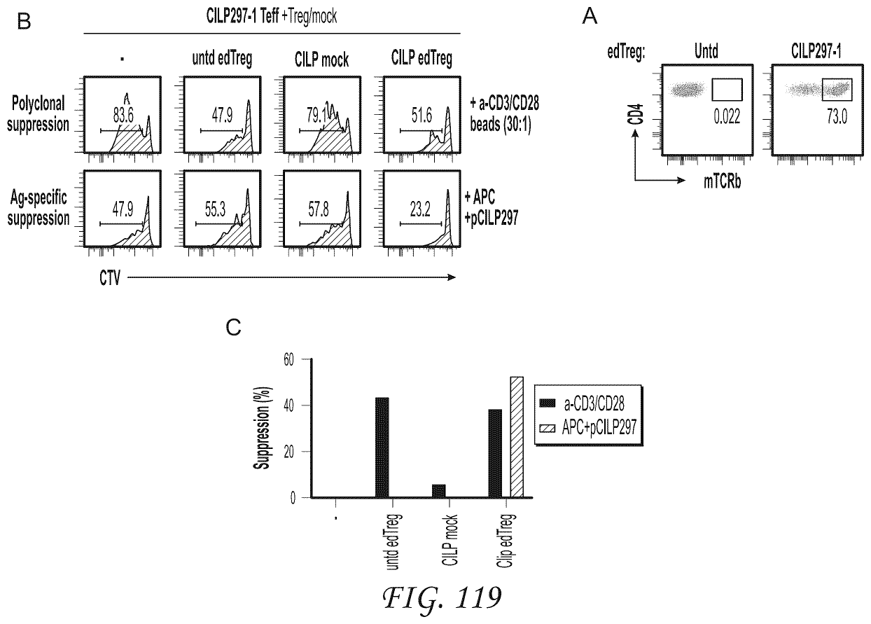

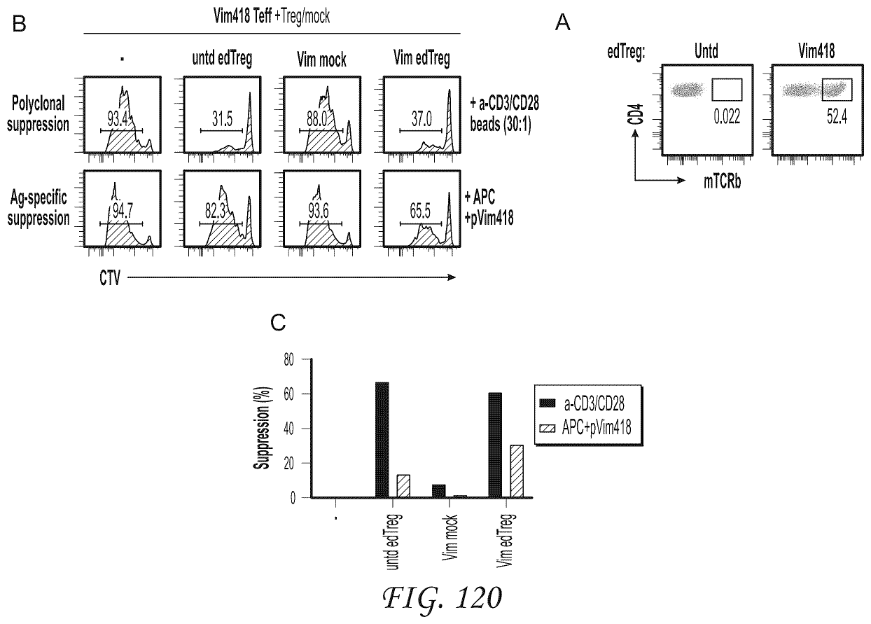

Suppressive activity of CILP-specific edTreg was determined. FIG. 119A depicts flow plots of mTCRb expression in untransduced edTreg and CILP297-1 edTreg gated on LNGFR+ Foxp3+ in edited cells transduced with no LV and LV CILP297-1-TCR, respectively. The CILP297-1 TCR had a specificity to a CILP297-311 epitope. FIG. 119B depicts a polyclonal suppression assay and an antigen-specific suppressing assay using CILP-specific edTreg. CILP-specific Teff cells were produced from LV CILP297-1-TCR transduction of CD4+ T cells and expanded for 15 days. For the polyclonal assay, CILP Teff were incubated with anti-CD3/CD28 beads with no Treg, untd edTreg, CILP edTreg, or mock cells. For the antigen-specific suppression assay, CILP Teff cells were co-cultured with APCs and CILP297 peptide in the presence of no Treg, untd edTreg, CILP edTreg, or mock cells. FIG. 119C depicts a graph of percentage suppression of CILP Teff proliferation by no Treg, untd edTreg, CILP edTreg, or mock in the presence of a-CD3/CD28 (black) or APC and CILP peptide (grey) calculated from percentage proliferation in FIG. 119B. Similar results were seen using CILP-specific edTregs. The CILP-specific edTregs showed antigen-specific and polyclonal suppressive function of antigen-specific T effector cells by in vitro suppression assays.

### Suppressive Activity of Vimentin-Specific edTreg

Suppressive activity of vimentin-specific edTreg was determined. FIG. 120A depicts flow plots of mTCRb expression in untransduced edTreg and Vim418 edTreg gated on LNGFR+ Foxp3+ in edited cells transduced with no LV and LV Vim418-TCR, respectively. The Vim418 TCR had a specificity to the epitope vimentin 418-431.

FIG. 120B depicts a polyclonal suppression assay and an antigen-specific suppressing assay using vimentin-specific edTreg. Vimentin-specific Teff cells were produced from LV Vim418 TCR transduction of CD4+ T cells and expanded for 15 days. For the polyclonal assay, Vim Teff were incubated with anti-CD3/CD28 beads with no Treg, untd edTreg, Vim edTreg, or mock cells. For the antigen-specific suppression assay, Vim Teff cells were co-cultured with APCs and Vim418 peptide in the presence of no Treg, untd edTreg, Vim edTreg, or mock cells. FIG. 120C depicts a graph of percentage suppression of Vim Teff proliferation by no Treg, untd edTreg, Vim edTreg, or mock in the presence of a-CD3/CD28 (black) or APC and Vimentin peptide (grey) calculated from percentage proliferation in FIG. 120B. The vimentin -specific edTregs showed antigen-specific and polyclonal suppressive function of antigen-specific T effector cells by in vitro suppression assays

### Antigen-Specific and Bystander Suppression of Aggrecan-Specific Teff

Antigen-specific suppression and bystander suppression of aggrecan-specific Teff was demonstrated with aggrecan-specific edTreg and vimentin-specific edTreg, respectively.

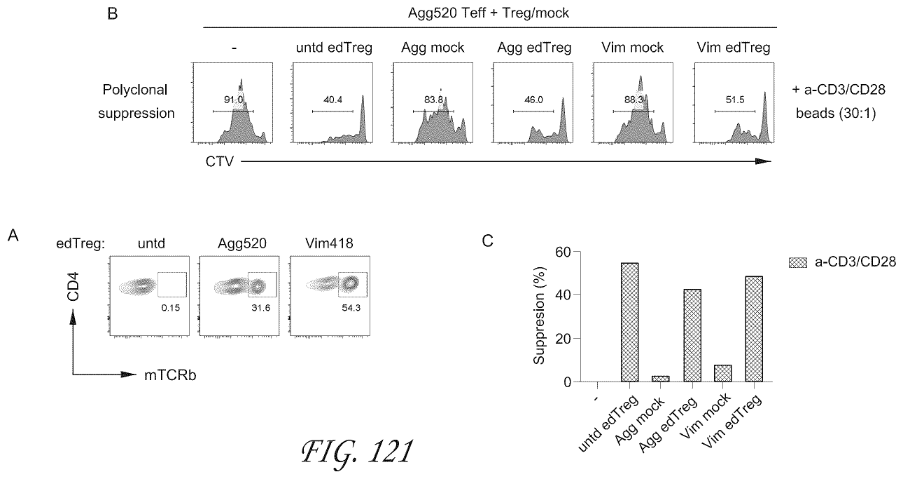

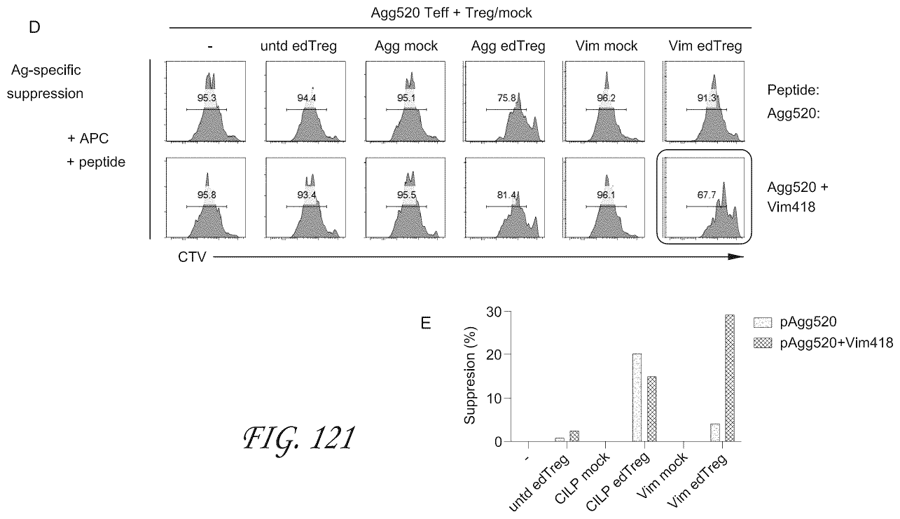

FIG. 121A depicts flow plots show mTCRb expression in untransduced, Agg520, and Vim418 edTreg gated on LNGFR+ Foxp3+ in edited cells transduced with no LV, LV Agg520-TCR, and LV Vim418-TCR, respectively. The Agg520 TCR has specificity to the epitope Aggrecan 520-539. FIG. 121B depicts a polyclonal suppression assay using Agg520 Teff and edTreg or mock specific to Agg520 or Vim418. Aggrecan-specific Teff cells were produced from LV Agg520-TCR transduction of CD4+ T cells and expanded for 15 days. Agg520 Teff were incubated with anti-CD3/CD28 beads with no Treg, edTreg, or mock. FIG. 121C depicts a graph of percentage suppression of Agg520 Teff proliferation by no Treg, untd edTreg, Agg edTreg/mock, or Vim edTreg/mock calculated from percentage proliferation in FIG. 121B. FIG. 121D depicts an antigen-specific and a bystander suppression assay using Agg520 Teff and edTreg or mock specific to Agg520 or Vim418. Agg520 Teff cells were co-cultured with no Treg, edTreg, or mock in the presence of APCs and Agg520 peptide or Agg520+Vim418 peptide. FIG. 121E depicts a graph of percentage suppression of Agg520 Teff proliferation by no Treg, edTreg or mock calculated from percentage proliferation in FIG. 121D. Significantly, bystander suppression of aggrecan-specific Teff by vimentin-specific edTreg was demonstrated.

### Antigen-Specific and Bystander Suppression of CILP-Specific Teff

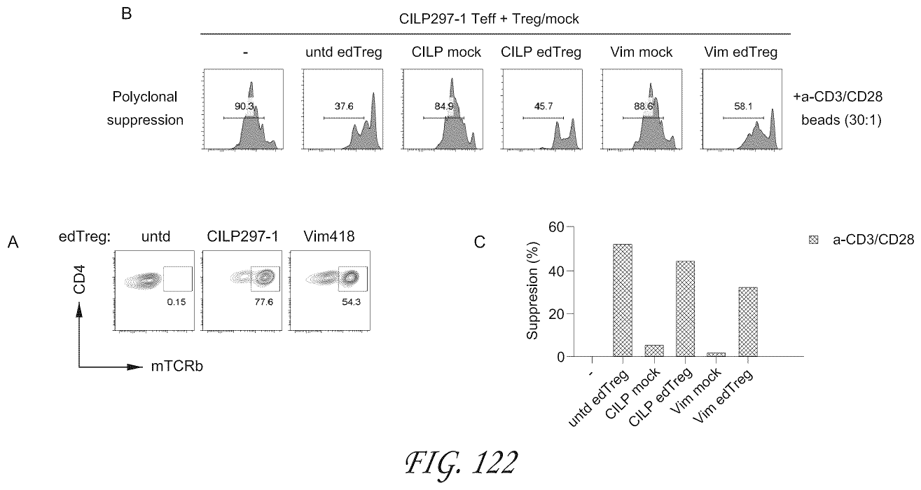

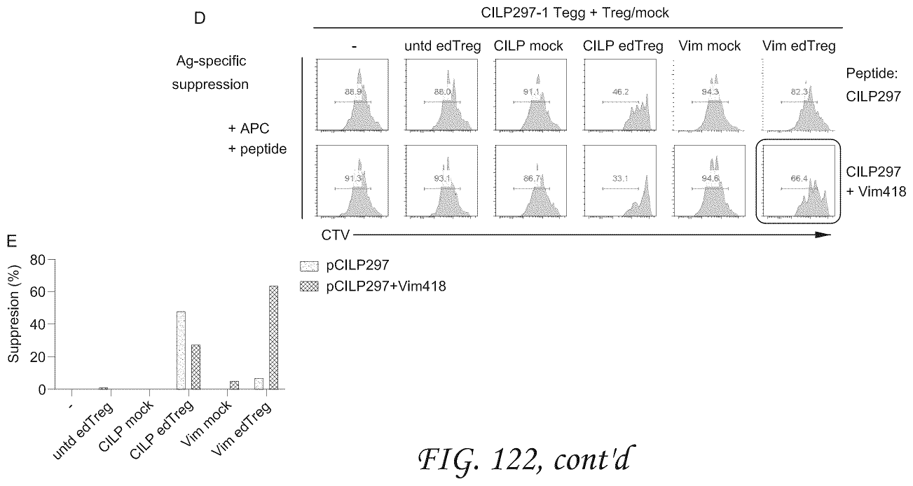

Antigen-specific suppression and bystander suppression of CILP-specific Teff was demonstrated with CILP-specific edTreg and vimentin-specific edTreg, respectively. FIG. 122A depicts flow plots of mTCRb expression in untransduced, CILP297-1, and Vim418 edTreg gated on LNGFR+ Foxp3+ in edited cells transduced with no LV, LV CILP297-1-TCR, and LV Vim418-TCR, respectively. FIG. 122B depicts a polyclonal suppression assay using CILP297-1 Teff and edTreg or mock specific to CILP297 or Vim418. CILP-specific Teff cells were produced from LV CILP297-1-TCR transduction of CD4+ T cells and expanded for 15 days. CILP297-1 Teff were incubated with anti-CD3/CD28 beads with no Treg, edTreg, or mock. FIG. 122C depicts a graph of percentage suppression of CILP Teff proliferation by no Treg, untd edTreg, CILP edTreg or mock, or Vim edTreg or mock calculated from percentage proliferation in FIG. 122B. FIG. 122D depicts an antigen-specific and bystander suppression assay using CILP297-1 Teff and edTreg specific to CILP297 and Vim418. CILP297-1 Teff cells were co-cultured with no Treg, edTreg, or mock in the presence of APCs and CILP297 peptide or CILP297+Vim418 peptide. FIG. 122E depicts a graph of percentage suppression of CILP Teff proliferation by no Treg, edTreg or mock calculated from percentage proliferation in FIG. 122D. Significantly, bystander suppression of CILP297-1 specific Teff by Vim edTregs was demonstrated.

### SLE-Specific edTreg and their Suppressive Activity

SLE-specific edTregs were generated. CD4 T cells were transduced with a SLE3 TCR, previously identified from a lupus patient, and the Foxp3 locus was edited.

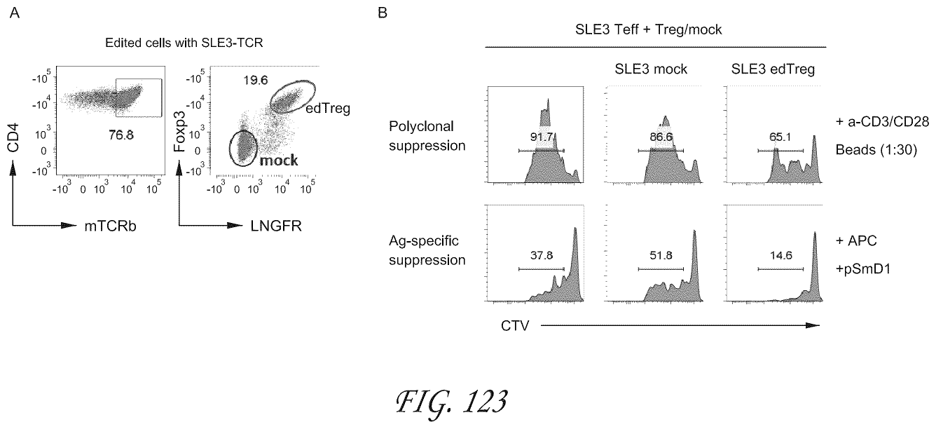

FIG. 123A depicts flow plots of mTCRb expression and LNGFR/Foxp3 expression in edited cells expressing SLE3-TCR on day 7. SLE3-TCR was previously identified from lupus patient. edTreg cells were enriched by LNGFR expression on day 10 and LNGFR− cells were used as mock cells for suppression assays. The SLE3-TCR had a specificity the epitope SmD1 65-80. FIG. 123B depicts a polyclonal suppression assay and an antigen-specific suppressing assay using SLE-specific edTreg. SLE-specific Teff cells were produced from LV SLE3-TCR transduction of CD4+ T cells and expanded for 15 days. For the polyclonal assay, SLE3 Teff were incubated with anti-CD3/CD28 beads with no Treg, SLE3 edTreg, or mock cells. For the antigen-specific suppression assay, SLE3 Teff were co-cultured with APCs and SmD1 peptide in the presence of no Treg, SLE3 edTreg, or mock cells. Edited cells expressed SLE-TCR and have polyclonal and antigen-specific suppressive activity.

Data in this Example demonstrated the ability to generate T1D, RA and SLE antigen-specific edTdreg, with suppressive activity supporting the use of antigen-specific edTreg therapies across a broad spectrum of autoimmune diseases.

### Example 13—Dual-Editing of Human CD4+ T Cells

Human CD4+ T cells were dual edited to generate edTreg to have an endogenous TCR knock-outed/inactivated, to be antigen-specific, and/or to be drug-selectable.

### Single Locus Approach

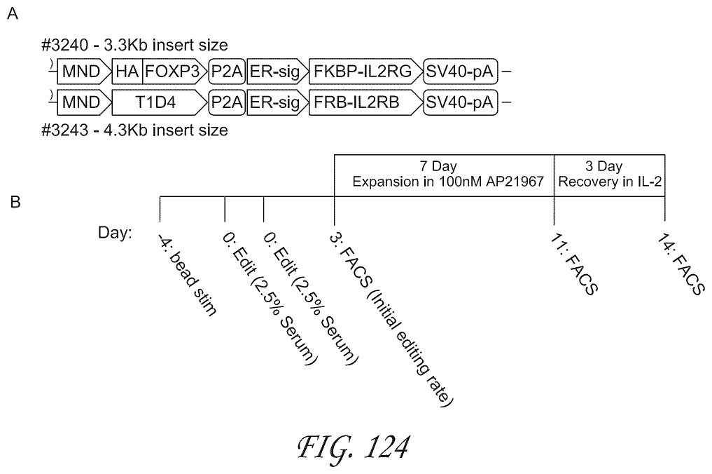

An IL-2 split-CISC system was used in a dual HDR editing strategy to provide efficient selection and enrichment of dual edited cells with endogenous TCR knockout. A challenge of the dual-editing approach is the ability to obtain sufficient numbers of edited cells for therapeutic use. This study aimed to increase cell viability during an expansion phase. The TRAC targeting AAV HDR-donor constructs used are depicted in FIG. 124A. AAV HDR-donor constructs were designed to introduce split-CISC elements into the TRAC locus using a single locus dual editing approach. CISC components were split between 2 constructs and co-expressed with either HA-FOXP3 or the T1D4 TCR (#3240 and #3243 respectively). Repair templates were flanked by homology arms matched to gRNAs targeting the TRAC locus. Only edited CD4+ T cells that incorporated both expression cassettes were predicted to selectively expand under Rapalog exposure.

A timeline for key steps for dual AAV editing of CD4+ T cells and expansion with Rapalog is depicted in FIG. 124B. The expansion protocol was adjusted from a 10-day expansion in AP21967 (a rapamycin analog) to 7 day expansion in AP21967 followed by a 3-day recovery in IL-2 containing medium. Briefly, human CD4+ T cells were edited using human TRAC gRNA_4, and #3240 (MND.HA.FOXP3.FKBP.IL2RG) and #3243 (MND.TID4.FRB.IL2RB) AAV constructs (single-locus dual editing). Immediately following electroporation, the cells were placed in 2.5% FBS containing media (recovery media) for ˜24 hours and then maintained in 20% FBS containing media throughout the rest of the experiment. FACS analysis was done on day 3 to determine editing rate and edited populations were cultured in the presence of either IL-2 or Rapalog for an additional 7 days to enrich dual FOXP3/T1D4 positive cells. Cells were allowed to recover for 3 days in media containing IL-2 prior to FACS analysis on day 14.

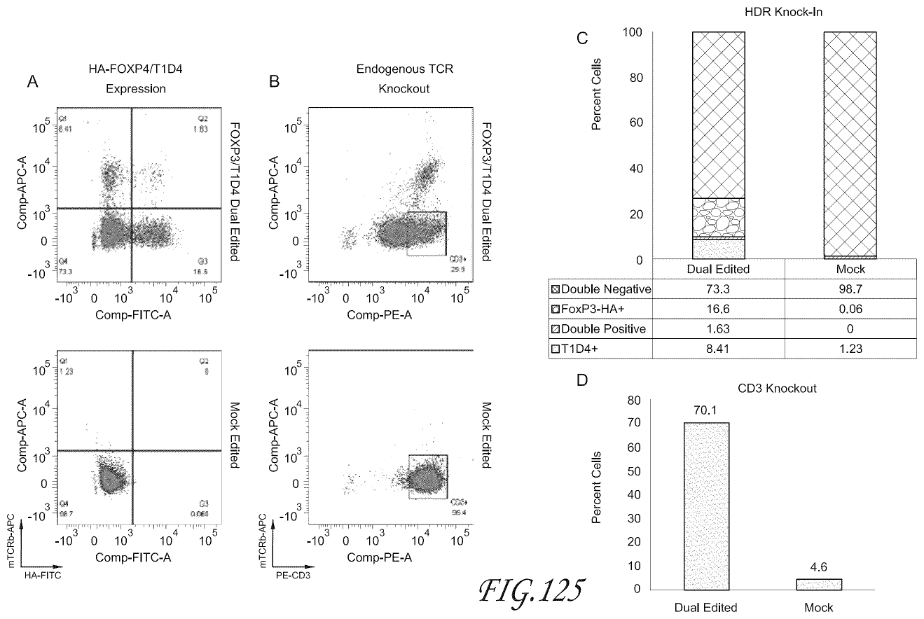

Dual editing of human CD4+ T cells using FOXP3 and T1D4 split-CISC constructs within the human TRAC locus resulted in FOXP3/T1D4 double positive cells and disrupted TCR expression. FIG. 125A depicts flow plots which show T1D4 and FOXP3 expression in mock edited, single edited and dual-edited cells (using 10% volume of both #3243 and #3240 AAV) at Day 3 post editing. Viral titers were 4.2E¹¹ and 1.3E¹² for #3243 and #3240, respectively. FIG. 125B depicts flow plots which show T1D4 and CD4 expression in mock edited, and mixed edited cells. FIG. 125C depicts histograms which show percent double negative, FOXP3-HA positive, T1D4 positive and FOXP3/T1D4 double positive cells within the dual edited cells. FIG. 125D depicts histograms which show percent CD3 knockout in FOXP3/T1D4 dual edited cells vs. mock edited cells. FACS analysis demonstrated an initial editing rate of 1.6% in T1D4/FOXP3 dual-edited cells compared to 0% in mock edited cells and CD3 knock-out (KO) of 70% in dual-edited cells.

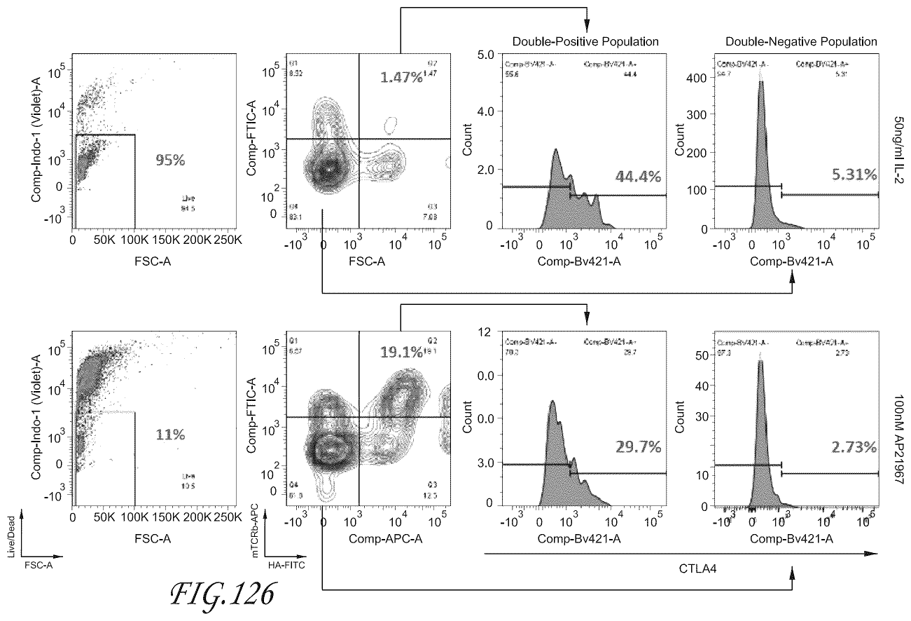

A robust enrichment of dual edited FOXP3/T1D4 expressing cells and increased CTLA4 expression was observed with AP21967 treatment of dual edited cells. TRAC locus dual-editing was performed as shown in FIG. 124A and FIG. 124B. FIG. 126A depict flow plots showing viability and T1D4 and FOXP3 expression in dual-edited cells treated with either 50 ng/mL IL-2 (upper panels) or 100 nM Rapalog (AP21967; lower panels) for 7 days. FIG. 126B depict flow plots showing CTLA4 expression of T1D4/FOXP3 double positive vs. double negative cell populations treated with either 50 ng/mL IL-2 (upper panels) or 100 nM Rapalog (AP21967; lower panels) for 7 days. FACS analysis following enrichment at day 7 showed a steady increase in FOXP3/T1D4 dual positive cells over time with 19.1% double positive cells at day 7 in AP21967 compared to 1.47% in IL-2.

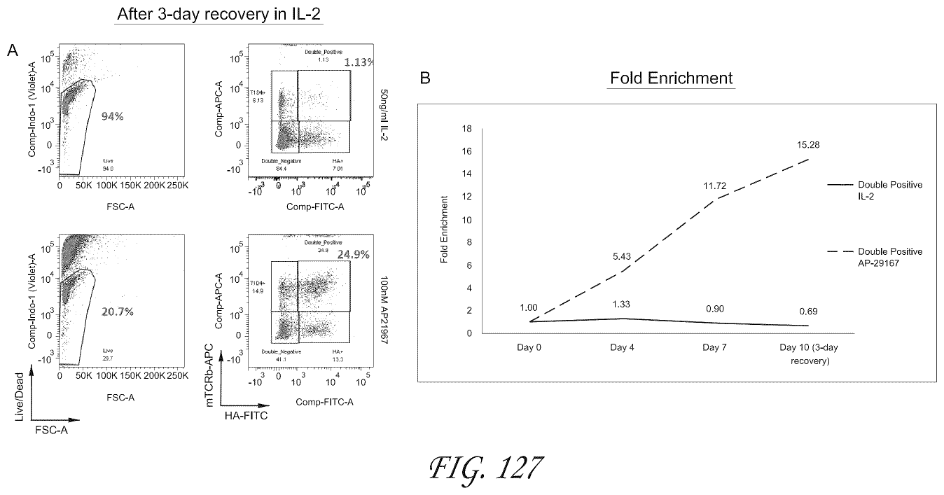

Cell viability in AP21967 declined in comparison to cells treated with 50 ng/mL IL-2 (11% viability vs. 95% viability respectively) (FIG. 126A). To improve cell viability following AP21967 treatment, cells were cultured in 50 ng/mL IL-2 containing medium following 7 days in AP21967. An improved viability and continued enrichment of dual edited FOXP3/T1D4 expressing cells was observed for cells treated with AP21967 following recovery in IL-2. TRAC locus dual-editing was performed as shown in FIG. 124A and FIG. 124B. Cells were analyzed at Day 10 following a 3-day recovery in IL-2 containing medium. FIG. 127A depicts flow plots showing viability (right plots) and T1D4 and FOXP3 expression (left plots) in dual-edited cells following treatment with 50 ng/mL IL-2 (upper plots) vs. 100 nM AP21967 (lower plots) after recovery in IL-2 medium. FIG. 127B depicts a graph showing fold enrichment of double positive T1D4/FOXP3 cells treated with either 50 ng/mL IL-2 or 100 nM Rapalog (AP21967) over a 10 day period with the last 3 days being in recovery media containing IL-2. Following recovery in IL-2, overall viability increased from 11% to 20.7% (FIG. 126A, FIG. 127A) and the percentage of double positive FOXP3/T1D4 cells continued to increase to 24.9%. Overall, the double-positive antigen-specific Treg population enriched approximately 15-fold over the course of this study (FIG. 127B), suggesting this may be an approach to improve viability and expansion.

To further characterize the T1D4/FOXP3 expressing cells, expression of CTLA4, a marker of FOXP3 expressing natural T regulatory cells (nTreg) was measured. FIG. 126B shows that double positive T1D4/FOXP3 expressing cells exhibited an increased expression of CTLA4 compared to the double-negative population consistent with a Treg-like phenotype.

This study further demonstrated that dual-editing can be used to introduce both a candidate TCR and the IL2 split-CISC cassette, and for enrichment using a Rapalog and generation of antigen-specific edTreg.

### Dual Editing Using Decoy-CISC (Split-DISC) Constructs

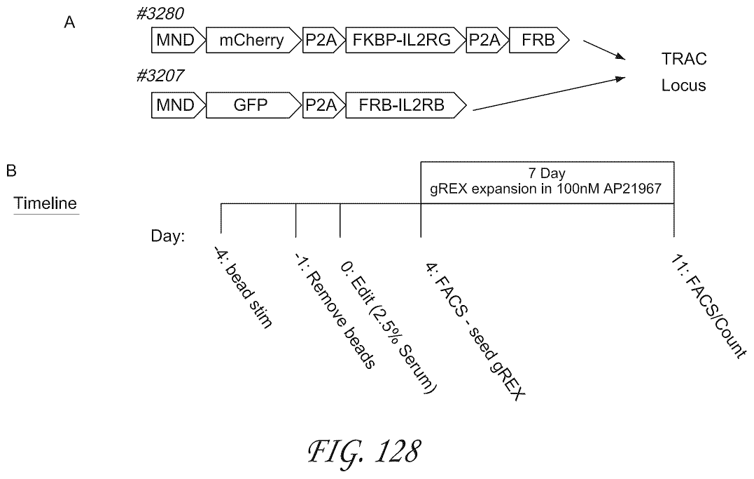

Rapamycin can be used in clinical studies using CISC-expressing edTreg. “Decoy-CISC” (DISC) constructs for efficient enrichment using either Rapamycin or AP21967 were tested. Split-DISC constructs were used to determine the enrichment and expansion of dual-edited T cells. The ability to scale up manufacturing to obtain cell numbers sufficient for animal studies by expanding edited CD4+ T cells in gREX flasks was assessed. In particular, dual-editing and enrichment of human CD4+ T Cells using split-DISC constructs was studied. Briefly, FIG. 128A depicts a split IL-2 DISC HDR knock-in construct (#3280), for selection of dual-edited cells in either Rapamycin or Rapalog. To generate the split decoy-CISC (split-DISC), the free FRB domain for cytoplasmic Rapamycin sequestration was added to the MND.mCherry.FKBP.IL2RG construct to generate (MND.mCherry.FKBP.IL2RG.FRB (#328)). Each repair template (#3280 and #3207) was flanked by identical homology arms matched to a gRNA targeting the TRAC locus. Edited CD4+ T cells incorporating one copy of each construct were predicted to selectively expand under Rapalog or Rapamycin treatment. FIG. 128B depicts a timeline of steps for dual AAV editing of CD4+ T cell using AAV #3280 and #3207, expansion with Rapalog/Rapamycin and analysis of enriched cells. Cells were bead stimulated (CD3/CD28) for 3 days prior to editing. Immediately following electroporation, the cells were placed in 2.5% FBS containing media (recovery media) for ˜24 hours and then maintained in 20% FBS containing media throughout the rest of the experiment. Three days post editing, cells were analyzed by flow for GFP and mCherry expression, and then expanded in media containing 50 ng/ml human IL-2 or 100 nM Rapalog.

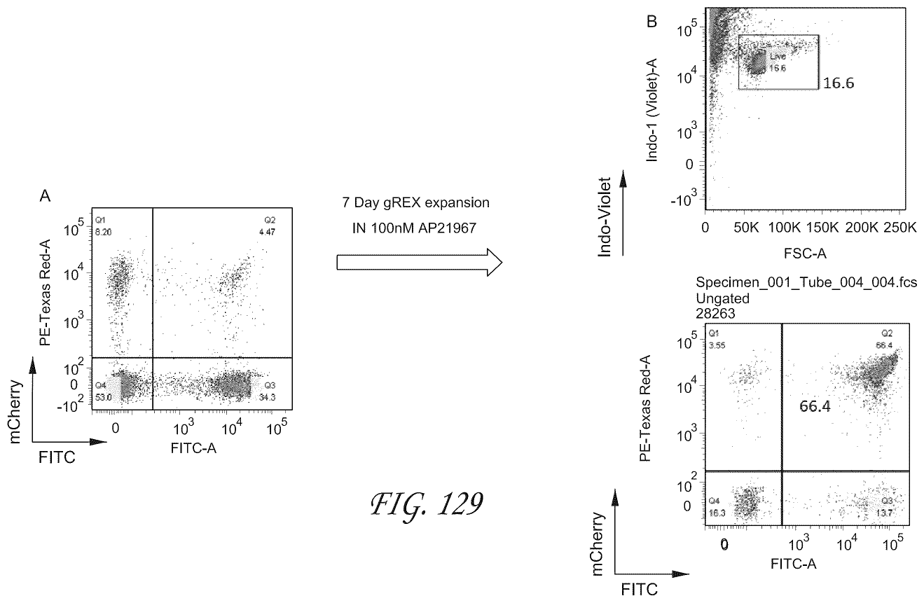

Dual editing of human CD4+ T cells using decoy-CISC (split-DISC) constructs and enrichment with AP21967 resulted in robust expansion of double positive cells. FIG. 129A depicts flow plots showing mCherry and GFP expression in dual edited cells (10% culture volume of #3280 and #3207 AAV donors, respectively) four days post editing. Viral titers were 3.30E+12 and 3.1E+10 for #3280 and #3207 respectively. FIG. 129B depicts flow plots showing viability (upper panel) and GFP and mCherry expression (lower panel) following the seeding of 7.6 million edited cells in gREX and 7 day expansion in the presence of AP21967 leading to 32-fold expansion of double-positive cells. The FACS analysis confirmed an initial editing rate of 4.47% mCherry/GFP double positive cells and enrichment to 66% mCherry/GFP double positive cells after 7 day expansion in gREX in the presence of AP21967. The results demonstrated a 32-fold expansion of double positive cells during the 7-day treatment in AP21967 resulting in a total of 11.1 million double positive cells from the original 340,000 cells seeded into gREX.

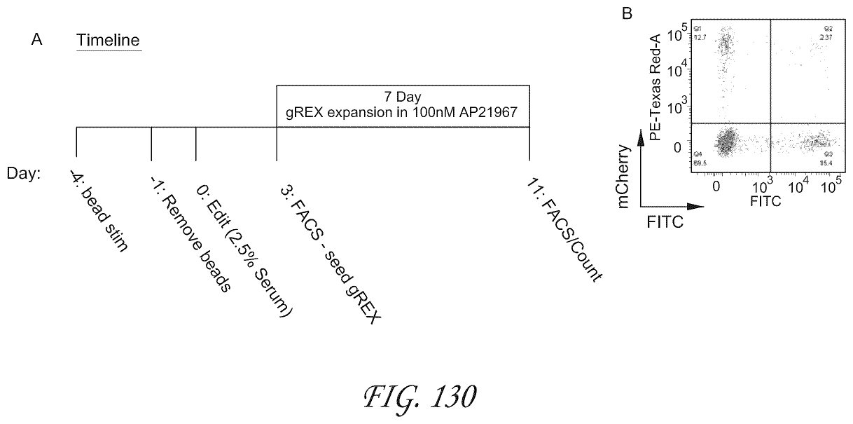

As second study was performed with a substantially similar protocol as immediately above. Dual editing of human CD4+ T cells using decoy-CISC (split-DISC) constructs and enrichment with AP21967 resulted in robust expansion of double positive cells. FIG. 130A depicts flow plots showing mCherry and GFP expression in dual edited cells (10% culture volume of #3280 and #3207 AAV donors, respectively) four days post editing. Viral titers were 3.30E+12 and 3.1E+10 for #3280 and #3207 respectively. FIG. 130B depicts flow plots show viability (upper panel) and GFP and mCherry expression (lower panel) following the seeding of 7.6 million edited cells in gREX and 7 day expansion in the presence of AP21967 leading to 32-fold expansion of double-positive cells.

Robust expansion of dual edited human CD4+ T cells using decoy-CISC (split-DISC) constructs was reproducible. FIG. 130A depicts a timeline of key steps for dual AAV editing of CD4+ T cell using AAV #3280 and #3207, expansion with Rapalog and analysis of enriched cells. Cells were bead stimulated (CD3/CD28) for 3 days prior to editing. Three days post editing, cells were analyzed by flow for GFP and mCherry expression, and then expanded in media containing 100 nM Rapalog for an additional 7 days. FIG. 130B depicts a flow plot showing mCherry and GFP expression in dual edited cells (10% #3280 and 10% #3207 AAV). Viral titers were 3.30E+12 and 3.1E+10 for #3280 MND.mCherry.FKBP.IL2RG.FRB and #3207 pAAV.MND.GFP.FRB.IL2RB respectively.

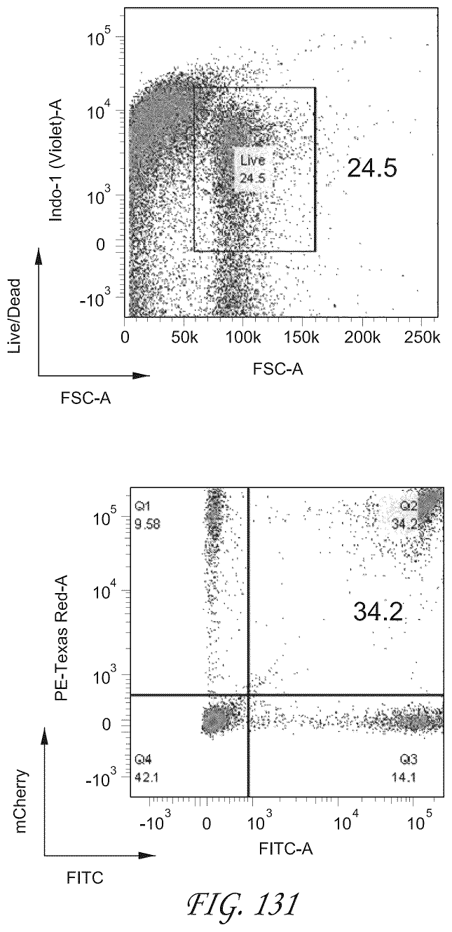

Expansion of dual edited human CD4+ T cells using decoy-CISC (split-DISC) constructs with AP21967 resulted in 45-fold increase in enriched cells. Cells were dual-edited as depicted in FIG. 130A. FIG. 131 depicts flow plots show viability and GFP and mCherry expression following the seeding of edited cells in gREX and 7 day expansion in the presence of AP21967. The total number of double positive cells in gREX at day 7 was 9.7 million, a ˜45-fold increase from the initial seeding of 216,000 double positive cells.

Importantly, these studies demonstrated that dual-editing strategy into the TRAC locus using the split DISC constructs provided at least a ˜45-fold expansion of dual edited cells. This level of expansion was similar to that observed using an all-in-one DISC constructs in a single editing event. Thus, this approach provides an efficient enrichment of dual-edited cells for in-vivo transplantation studies and ultimately clinical application.

### Ag-Specific Treg Mouse Studies

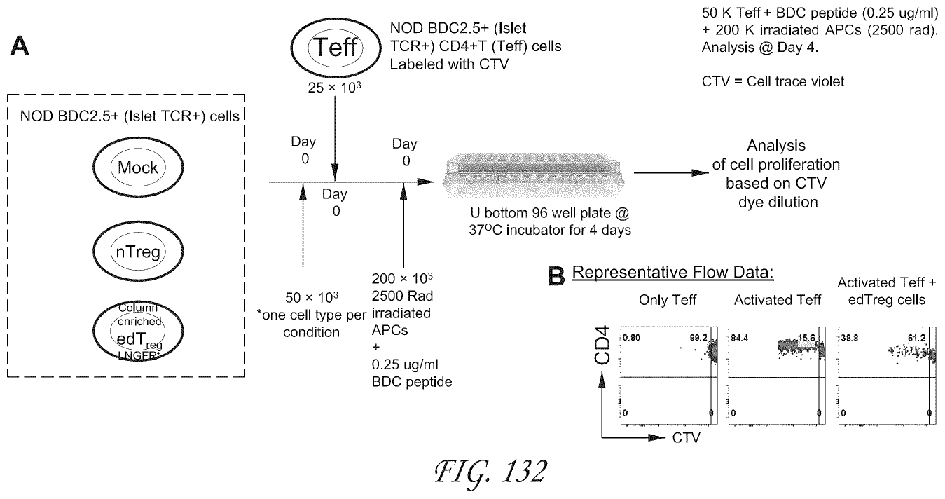

To assess functional activity of mouse Ag-specific edTreg cells, an antigen-specific in vitro suppression assay was established. The proliferation of BCD2.5 (islet-antigen specific) Teff was assessed. The BCD2.5 (islet-antigen specific) Teff were activated by a BDC peptide in the presence and absence of BCD2.5-expressing MND.LNGFR.P2A edTregs or purified BCD2.5 TCR expressing nTreg. Briefly, FIG. 132A depicts an in vitro suppression assay using mouse edTreg or nTreg. MND.LNGFR.p2A (#3261) edited Treg were enriched by anti-LNGFR column at day 2 post editing and resuspended into RPMI media containing 10% FBS. nTreg (CD4+CD25+), Teff (CD4+CD25+) and antigen presenting cells (CD4+CD25+) were isolated from the spleen and lymph nodes cells of 8 to 10 weeks old NOD BDC2.5+ mice by column enrichment. Enriched 5×10⁶ Teff were resuspended in 2 ml of PBS and labeled with cell trace violet for 15 minutes at 37° C. and then washed and resuspended in media before their addition in suppression assay. To setup this assay, 2.0×10⁵ irradiated APCs (2500 rad) were loaded with 0.25 μg/ml BDC peptide together with 0.5×10⁵ Teff and titrated numbers of BDC2.5+ nTreg or edTreg in a U bottom 96 well tissue culture plate with total volume of 250 μl media. Cells were incubated at 37° C. in CO₂ incubator of four days. At day 4 cells were washed twice with PBS and stained with live/dead, anti-CD4, anti-CD45 and CD25, and analyzed by FACS (LSRII) for the suppression of Teff proliferation by Treg. FIG. 132B depicts representative flow data obtained showing a reduction of BDC2.5+ Teff proliferation in the presence of BDC2.5+ edTreg cells. This demonstrated suppression of peptide-activated Teff cells in the presence of edTregs.

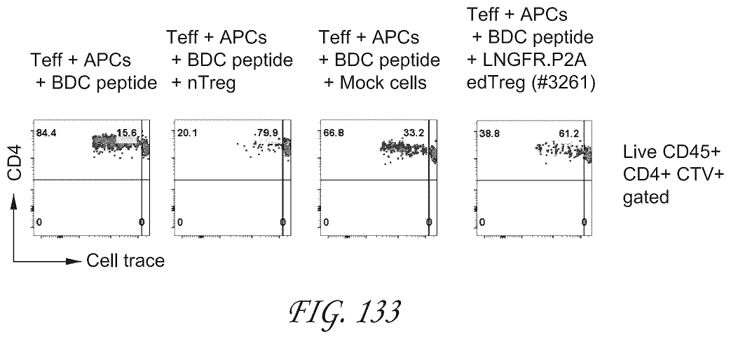

An in vitro suppressive function of murine BDC2.5+ nTreg and edTreg was observed. FIG. 133 depicts flow cytometry plots showing cell trace violet labeled CD4+ T cells in the presence and absence of mock, MND.LNGFR.p2A (#3261) edited Treg or nTregs from NOD BDC2.5+ mice. Numbers in each flow plots indicated the proportion of proliferating vs non-proliferating cells, respectively. Murine edTreg and nTregs exhibited robust in vitro suppressive function. These data demonstrated that edTreg cells expressing the LNGFR selectable marker (MND.LNGFR.P2A #3261) exhibited antigen-specific suppressive activity in vitro.

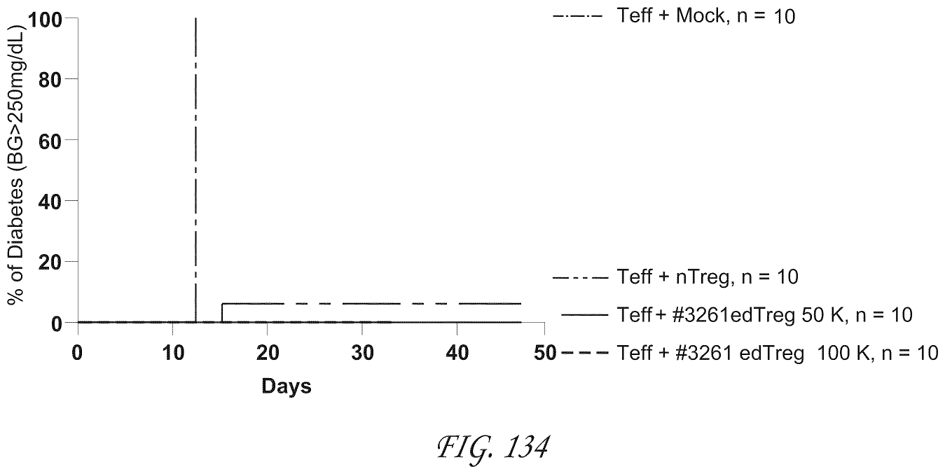

In vivo activities of edTreg were examined with methods substantially similar to those in Examples 6 and 11. In particular, antigen specific T cell function was examined in an NSG adoptive transfer model in which nTregs and column enriched edTregs were compared. Engineered BDC2.5+ antigen-specific (BDC) edTregs, or antigen-specific nTregs were infused into the mice followed by infusion of antigen-specific Teff cells. Mice were monitored for diabetes up to 49 days. FIG. 134 depicts a graph showing the percent of diabetic mice after receiving effector cells plus the designated mock edited, MND.LNGFR.P2A edited or nTreg cells from NOD BDC2.5 mice. Column enriched Ag-specific MND.LNGFR.P2A edTregs completely prevented diabetes in NSG mice and exhibited comparable function to nTregs.

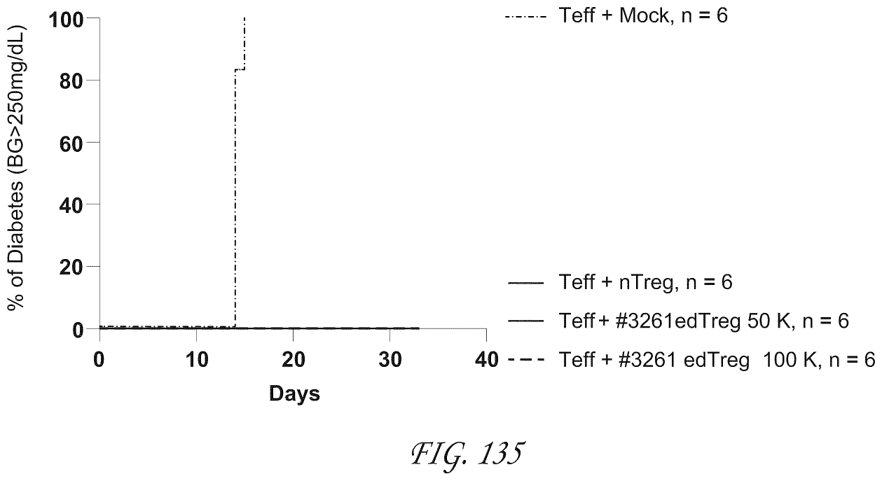

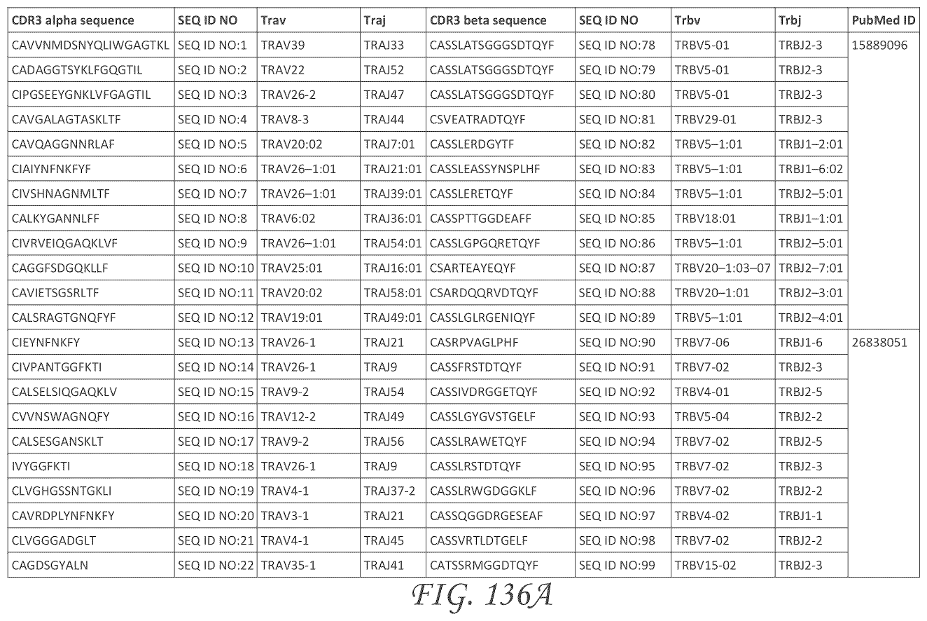

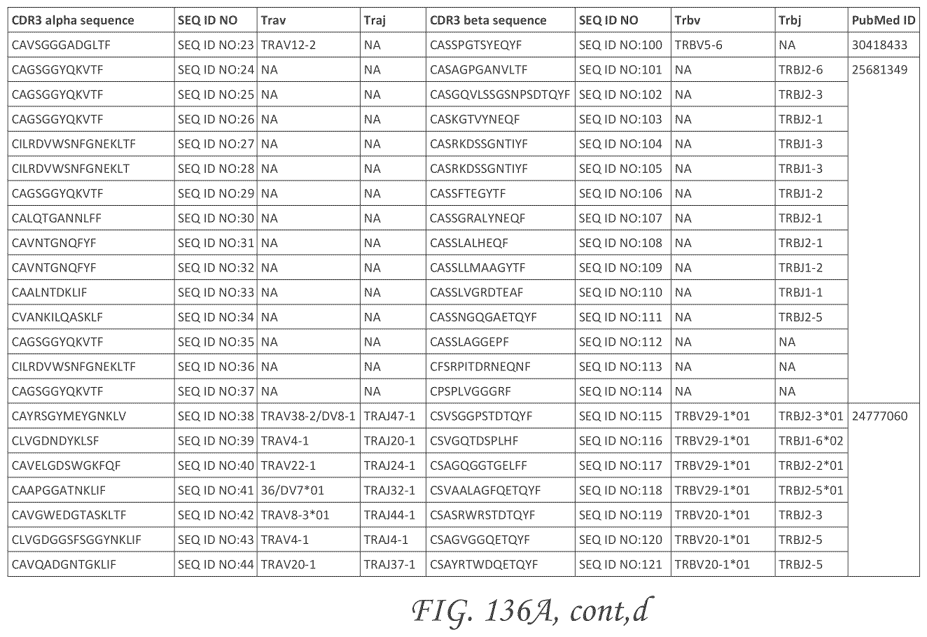

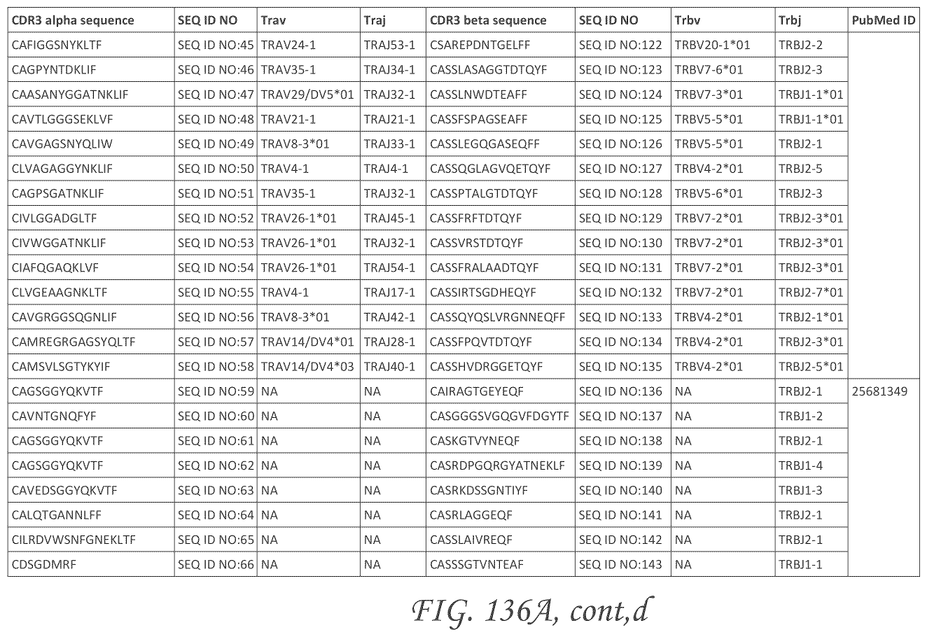

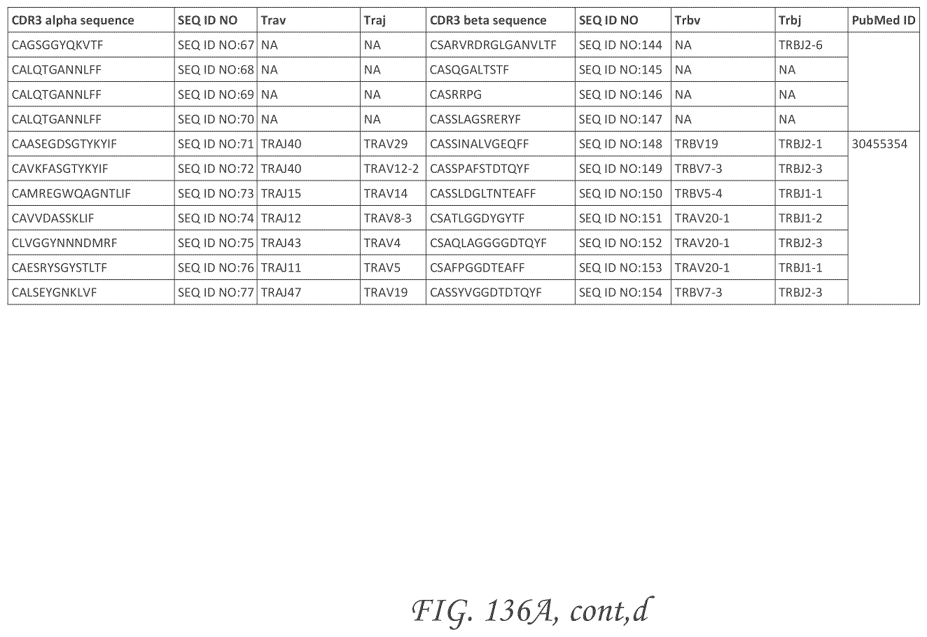

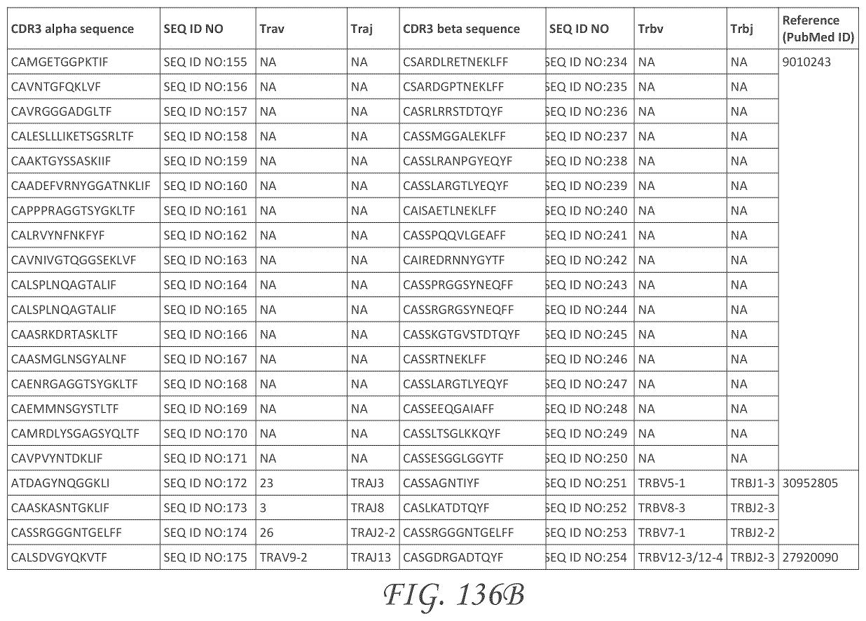

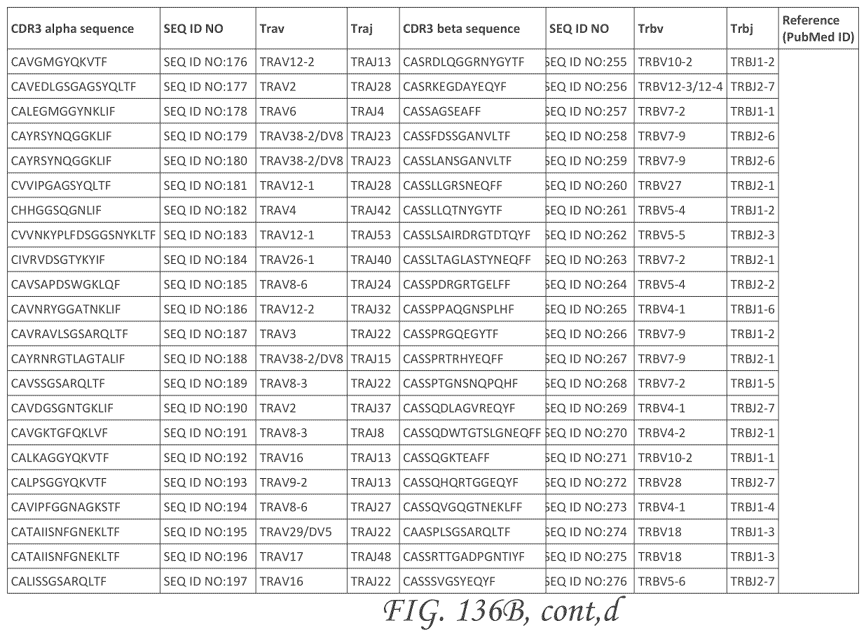

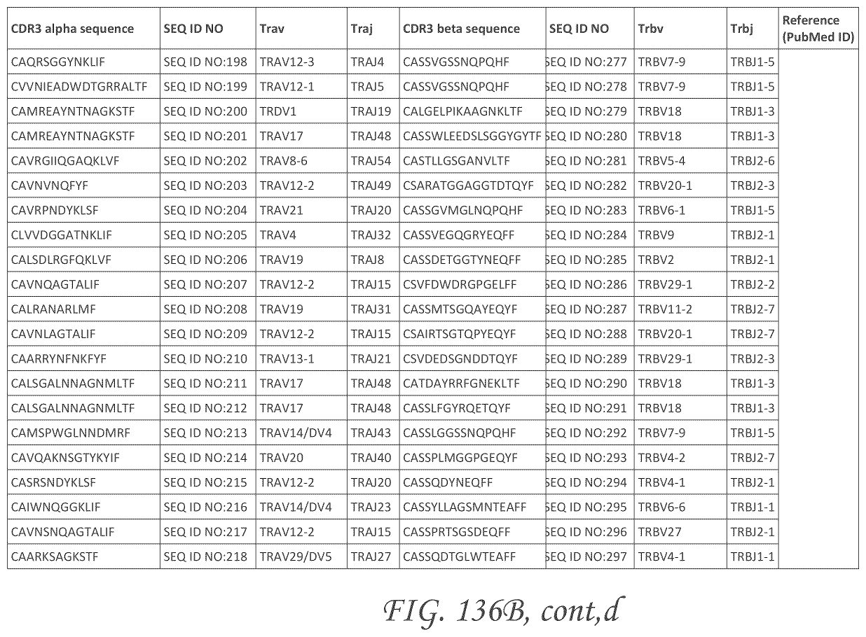

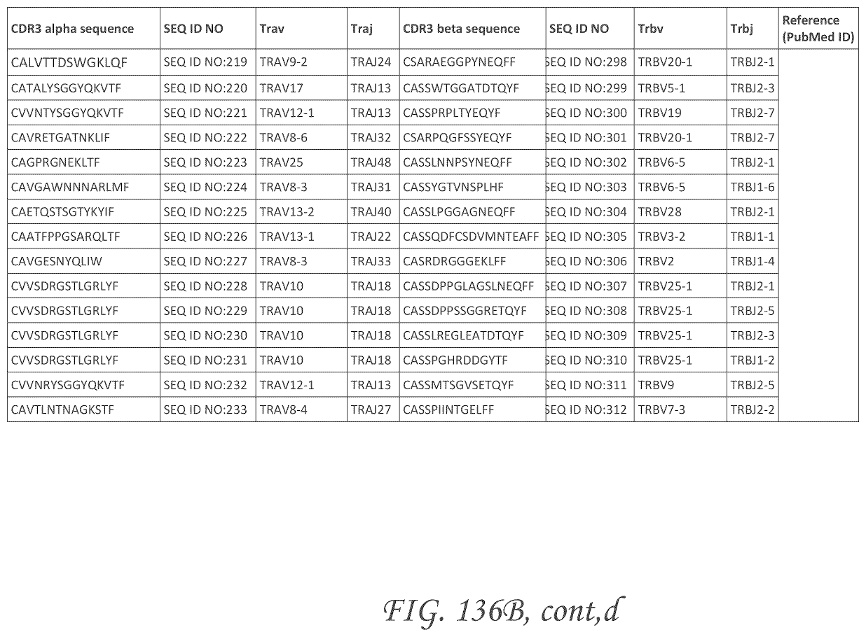

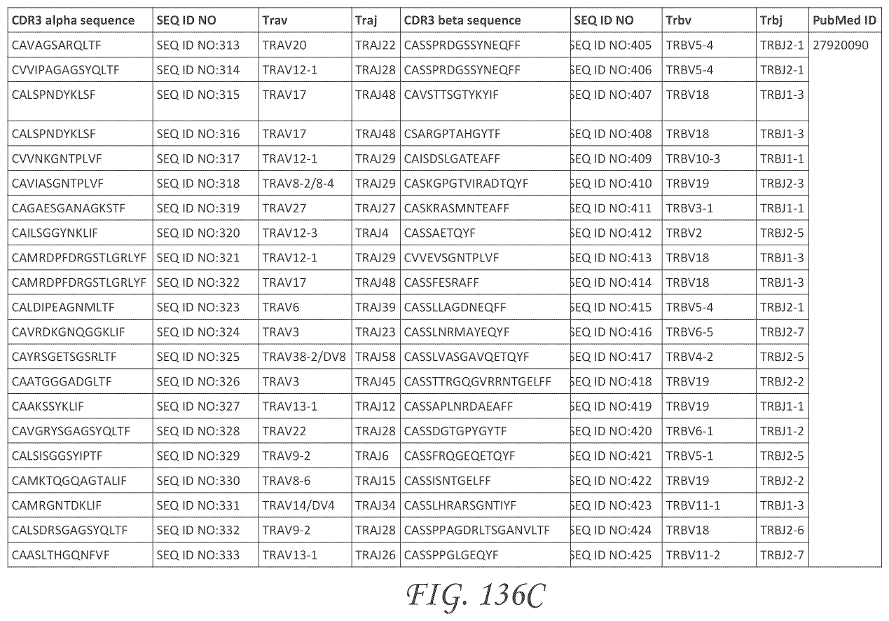

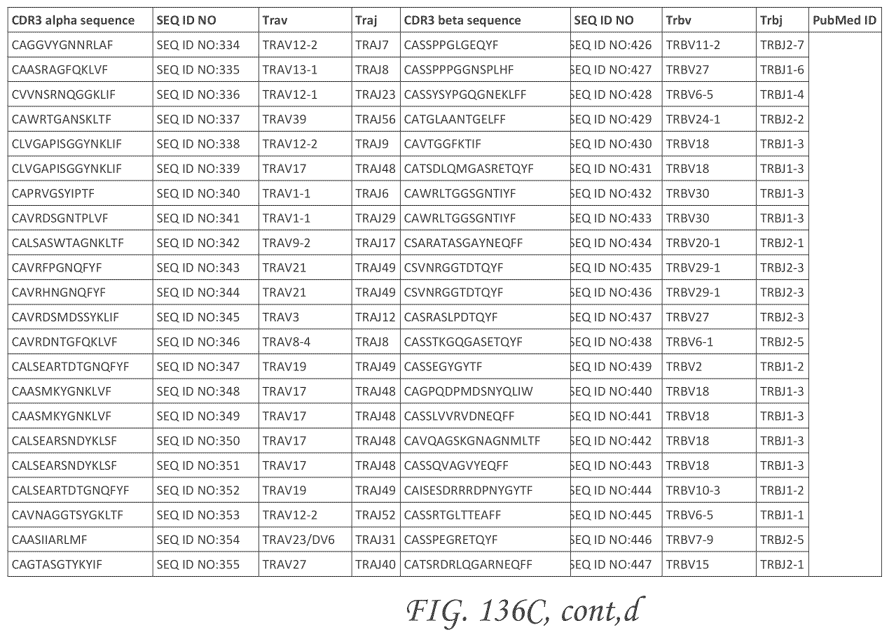

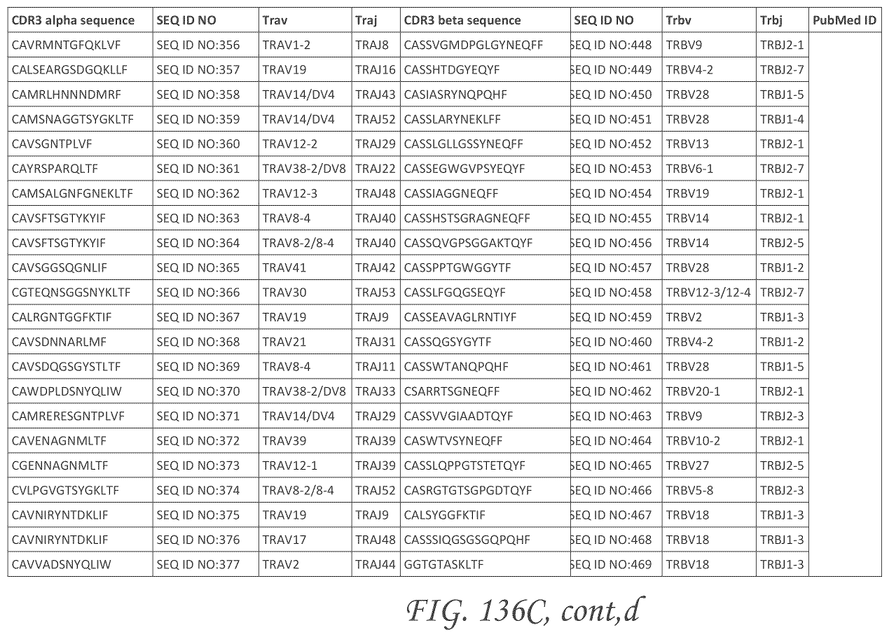

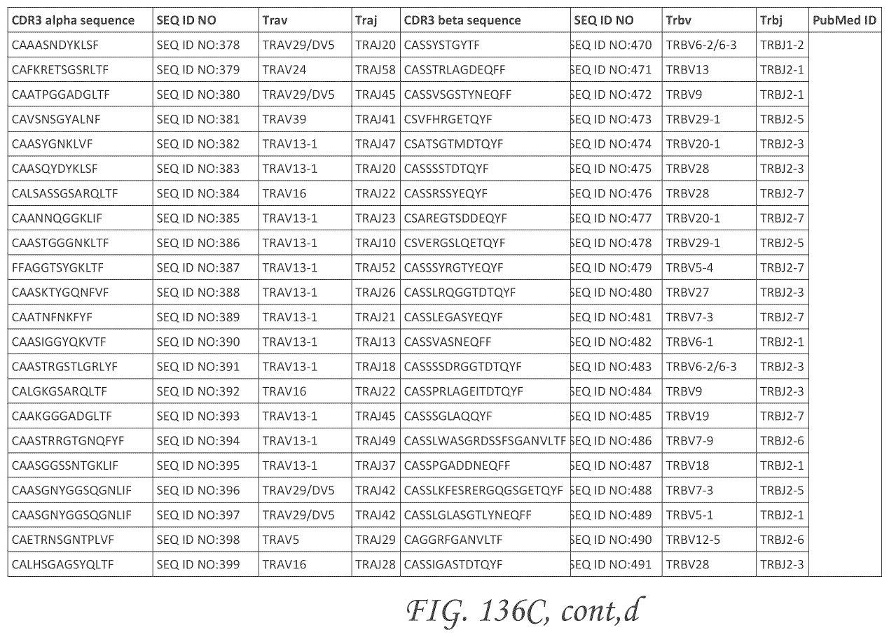

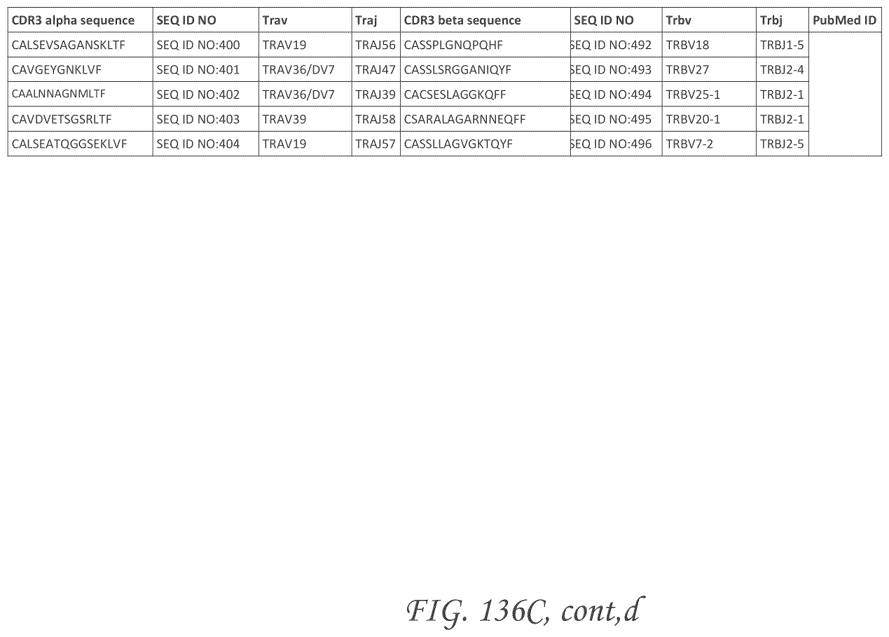

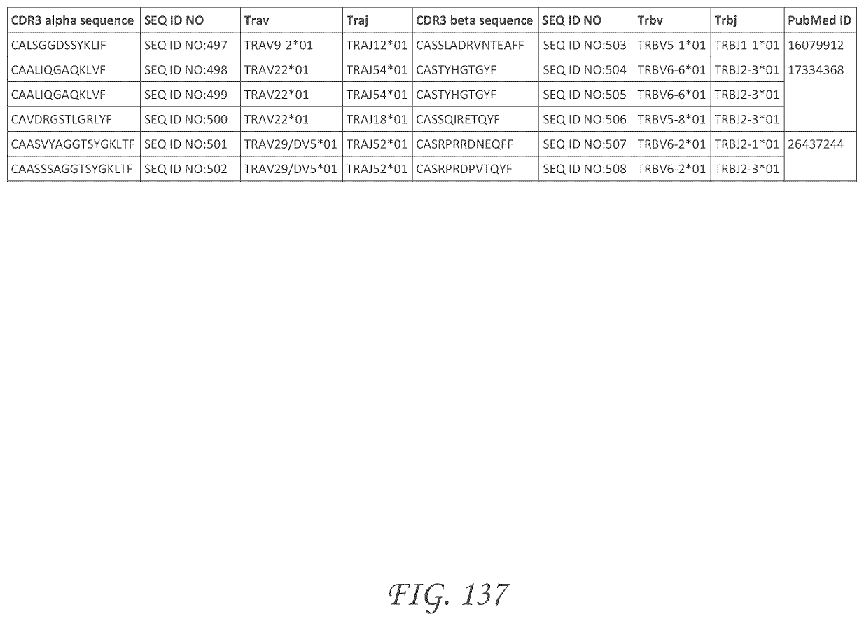

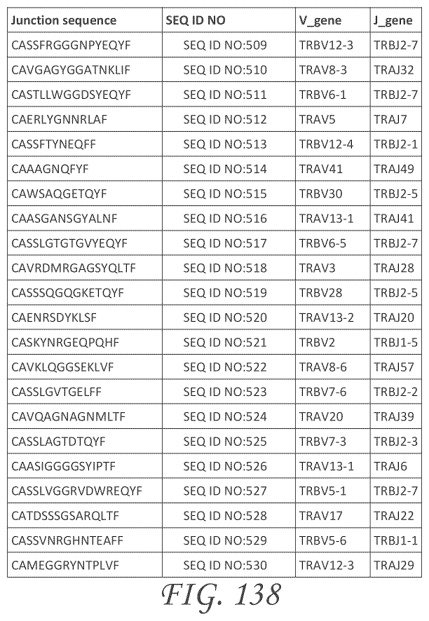

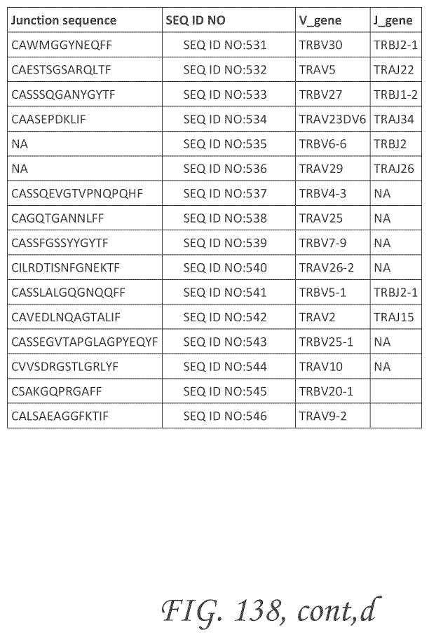

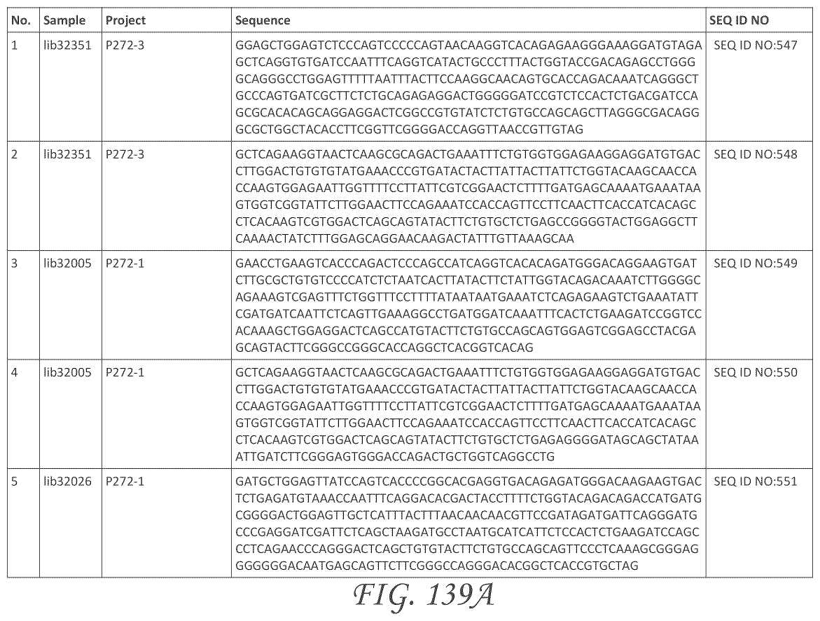

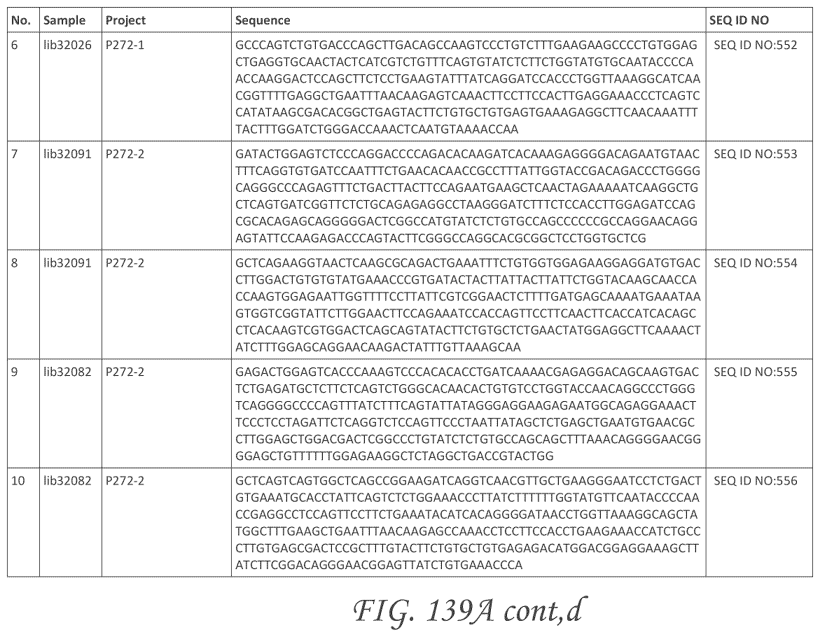

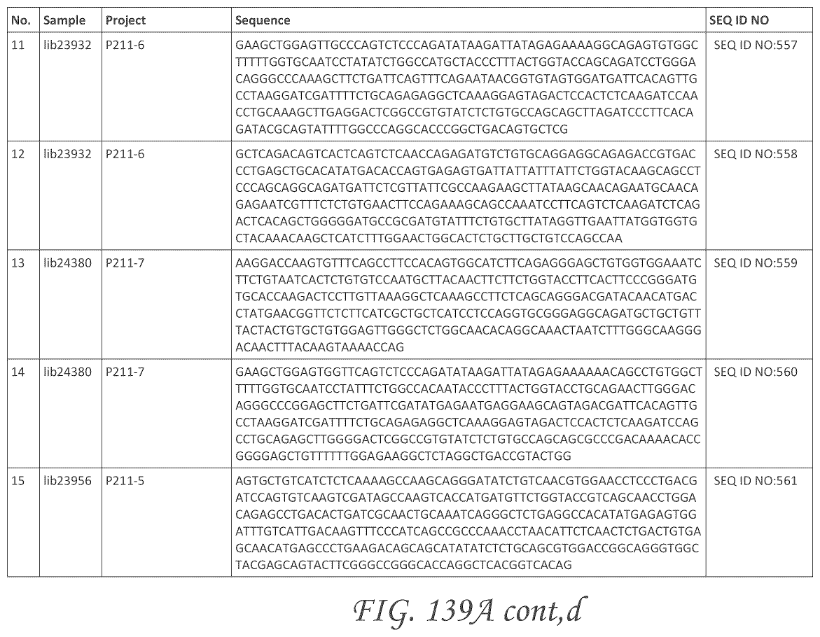

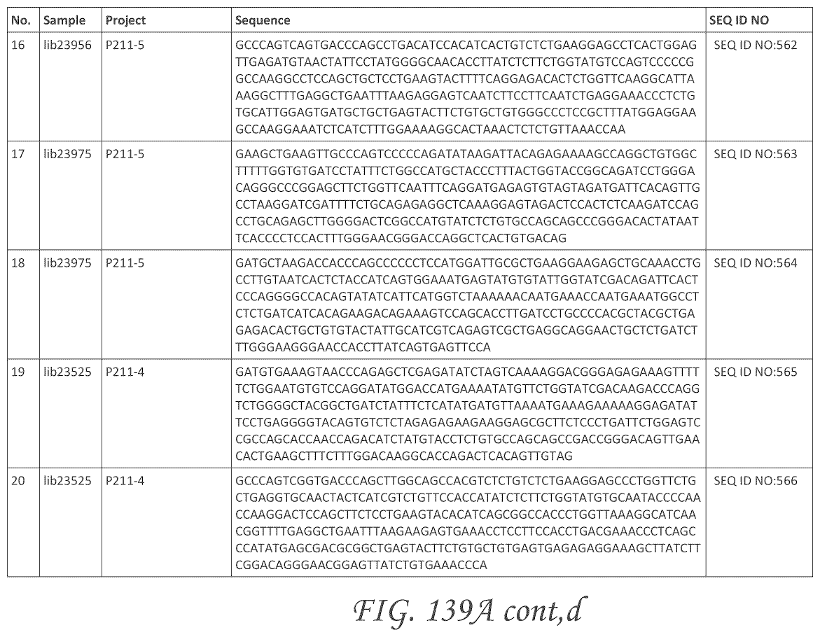

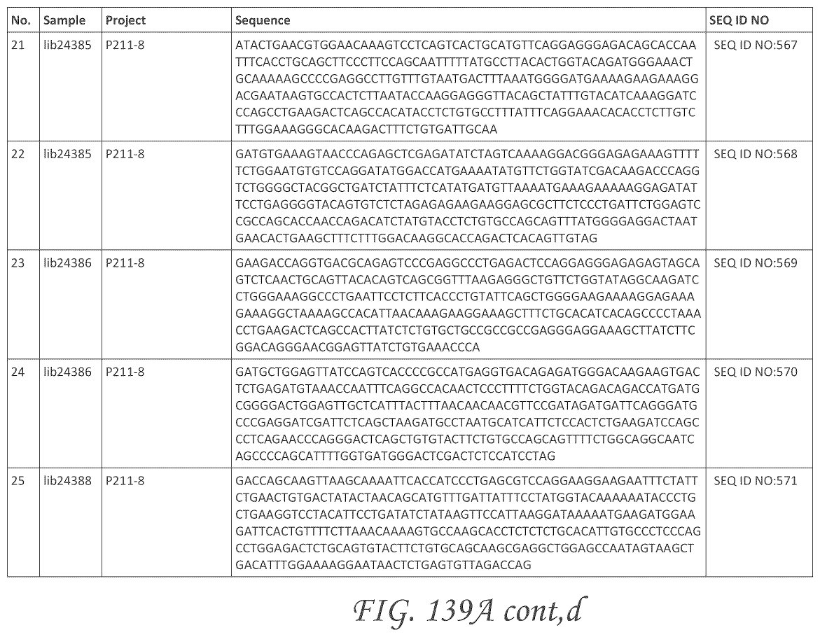

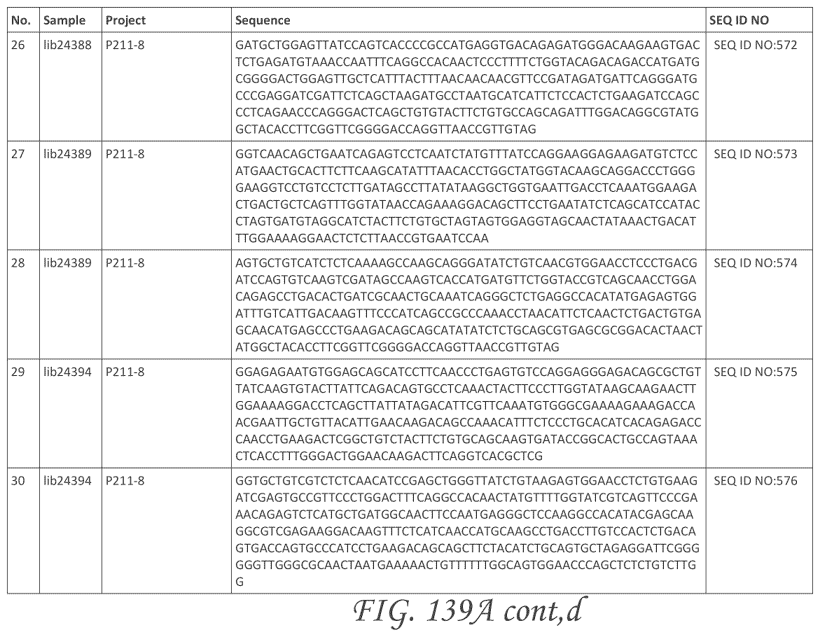

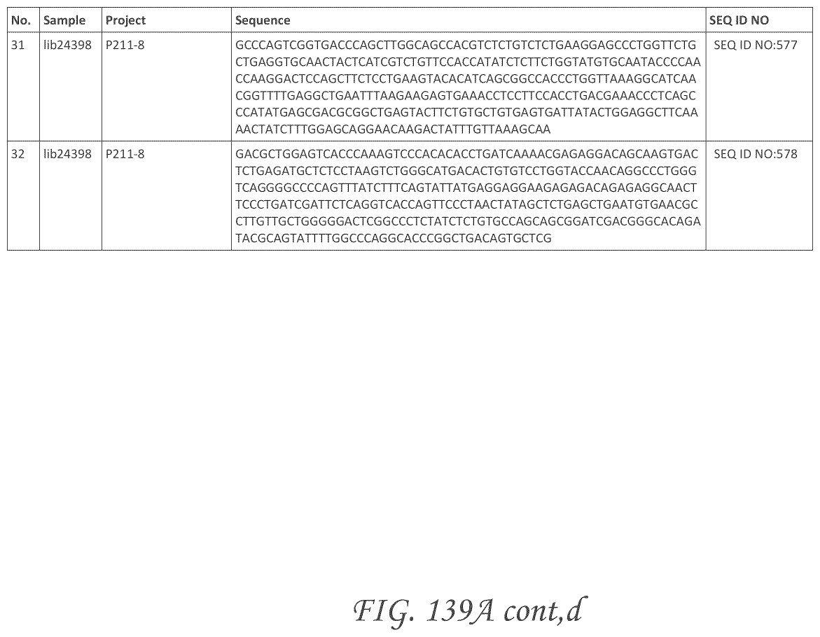

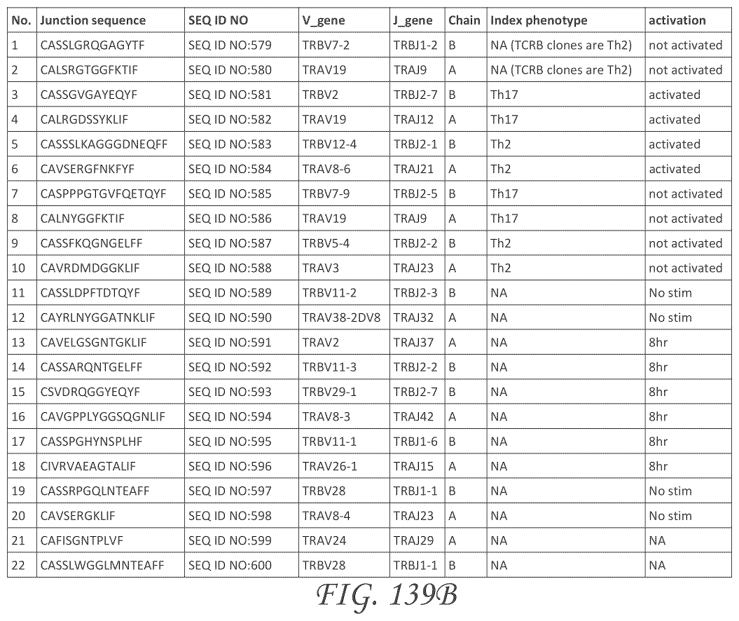

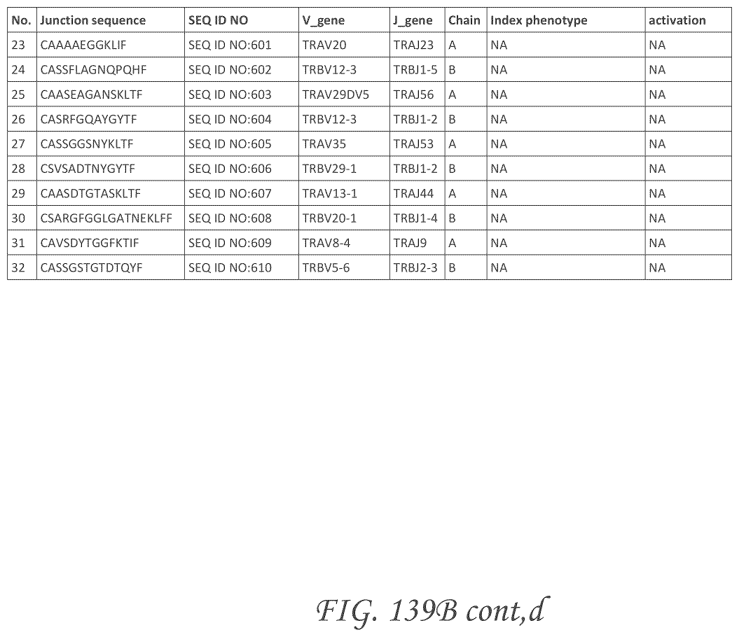

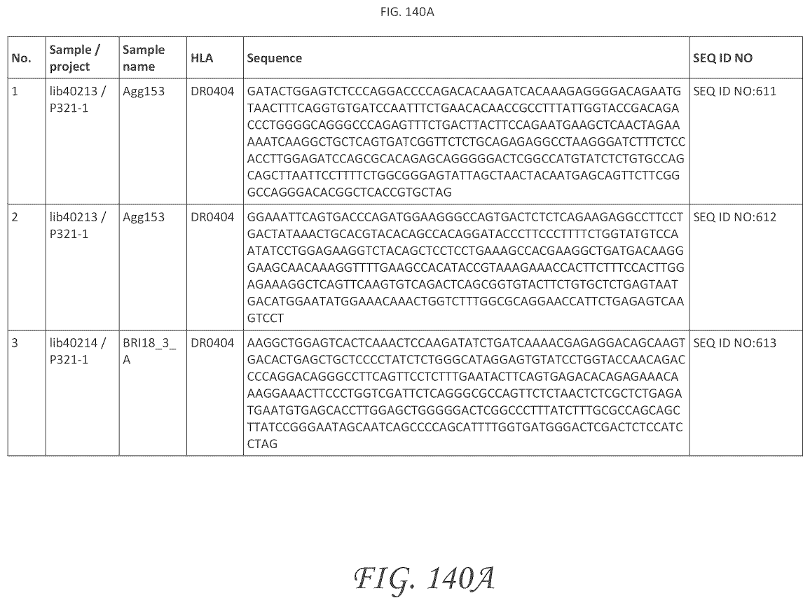

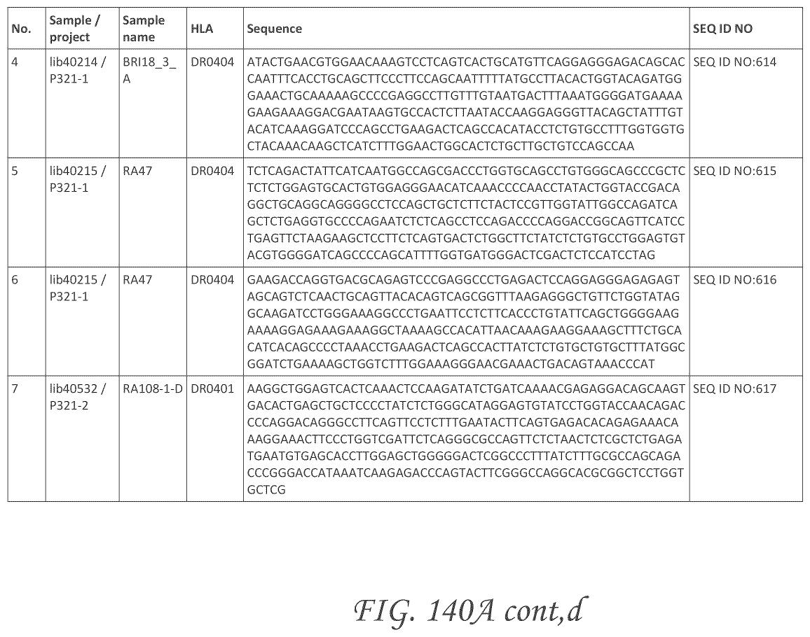

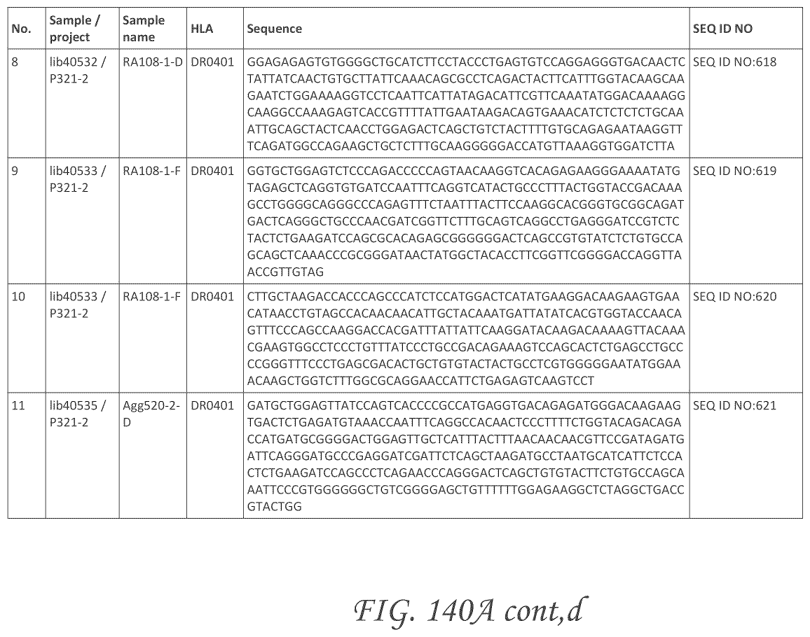

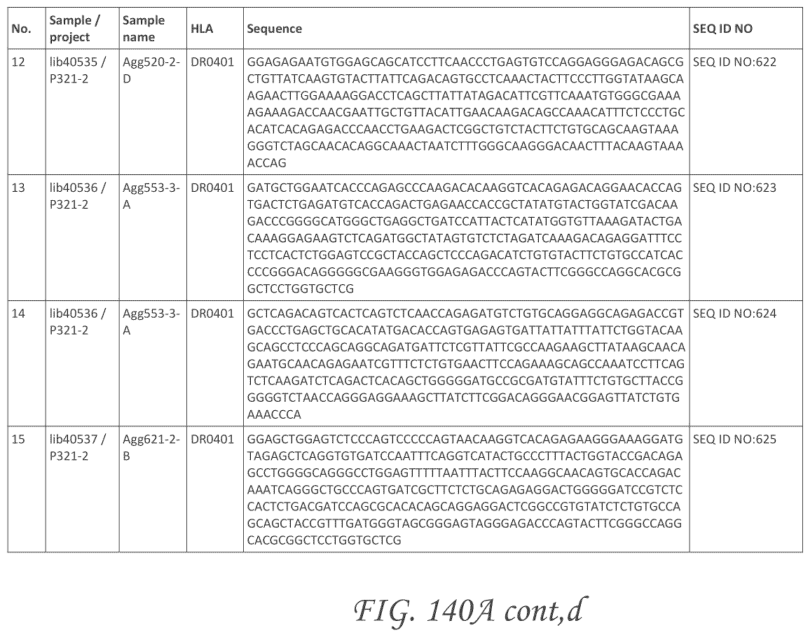

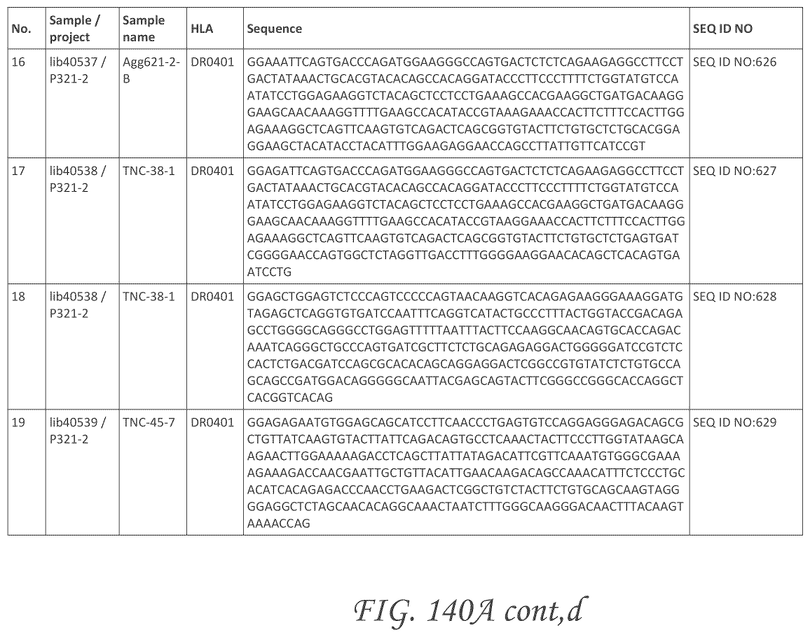

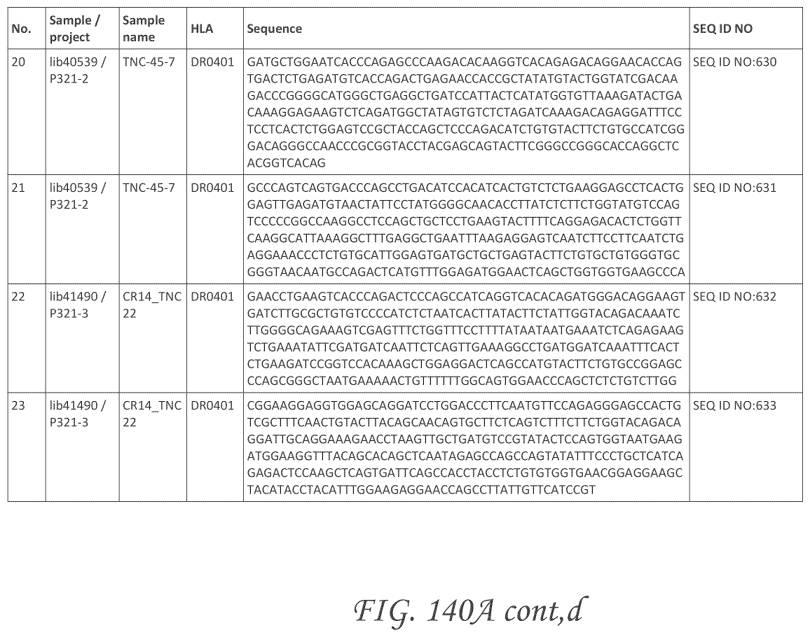

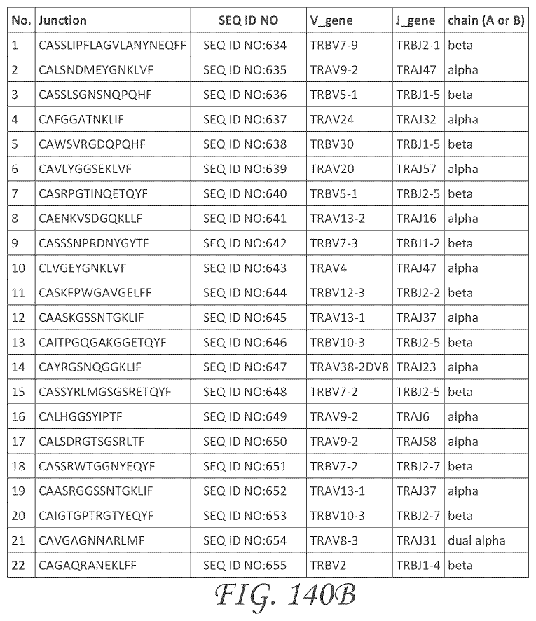

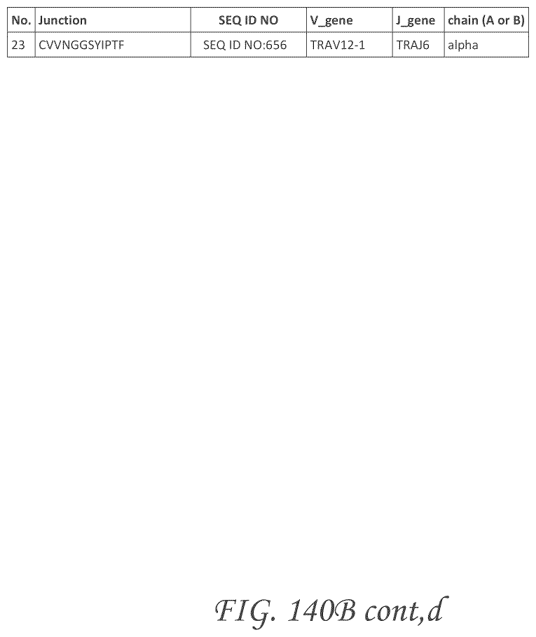

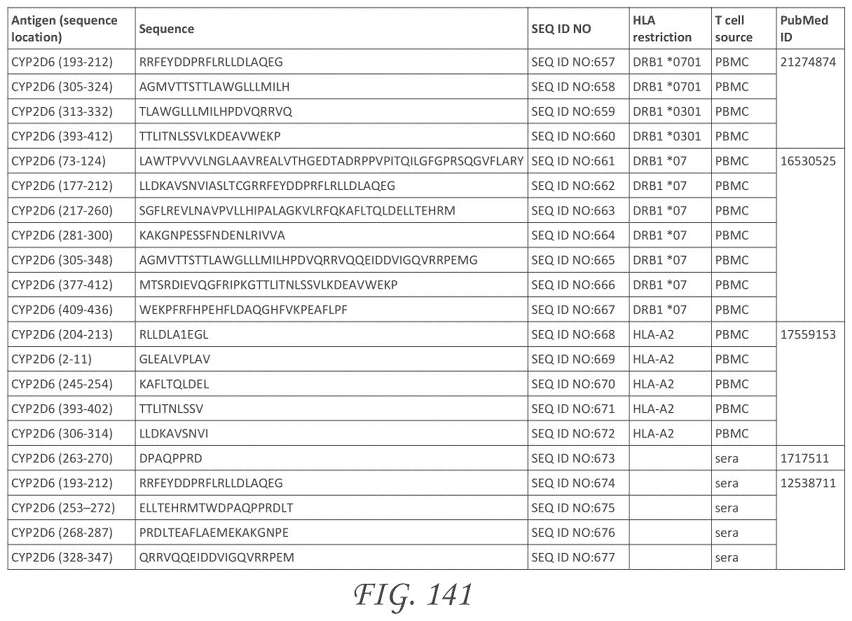

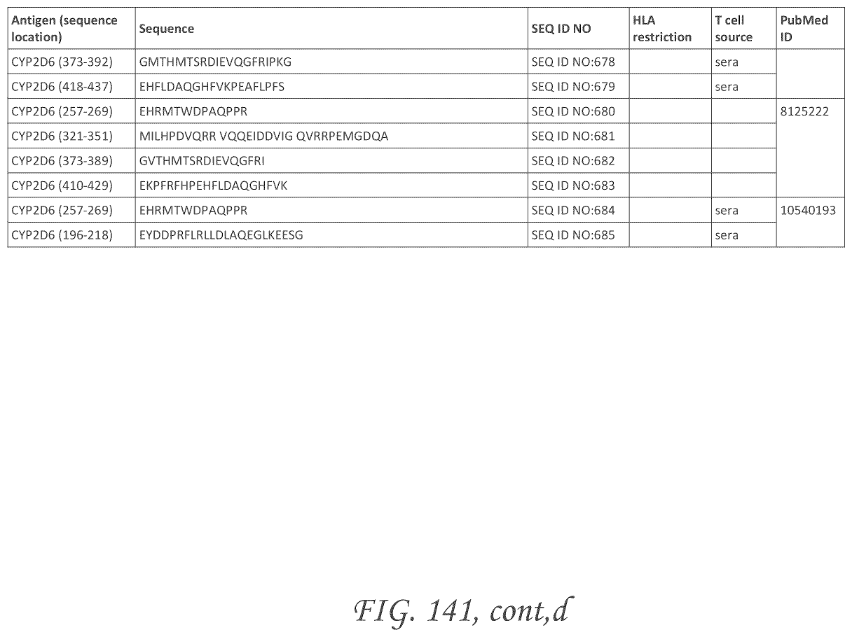

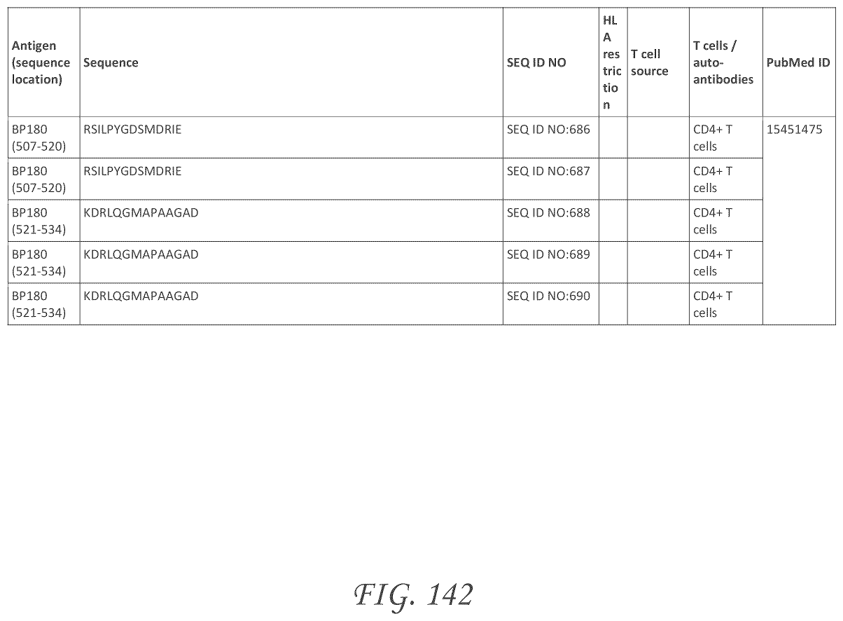

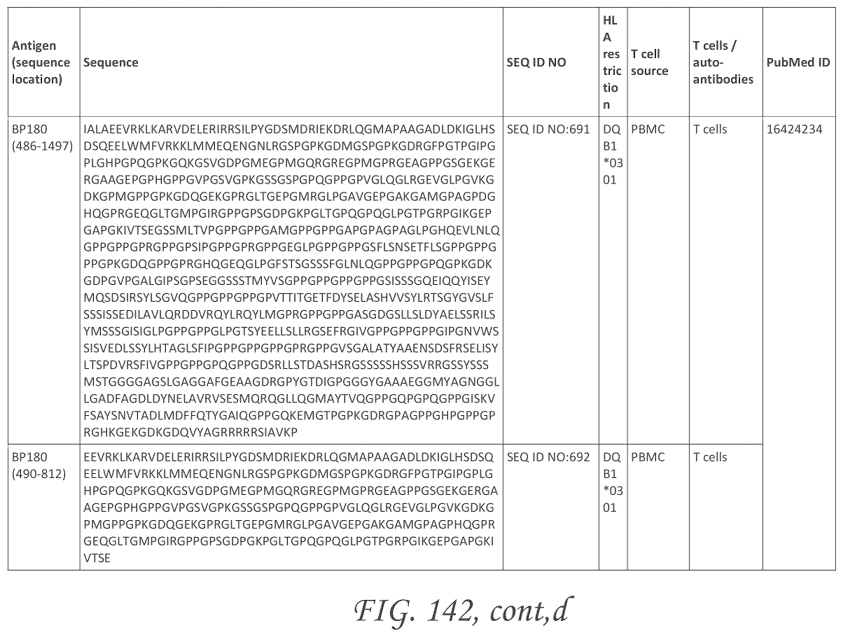

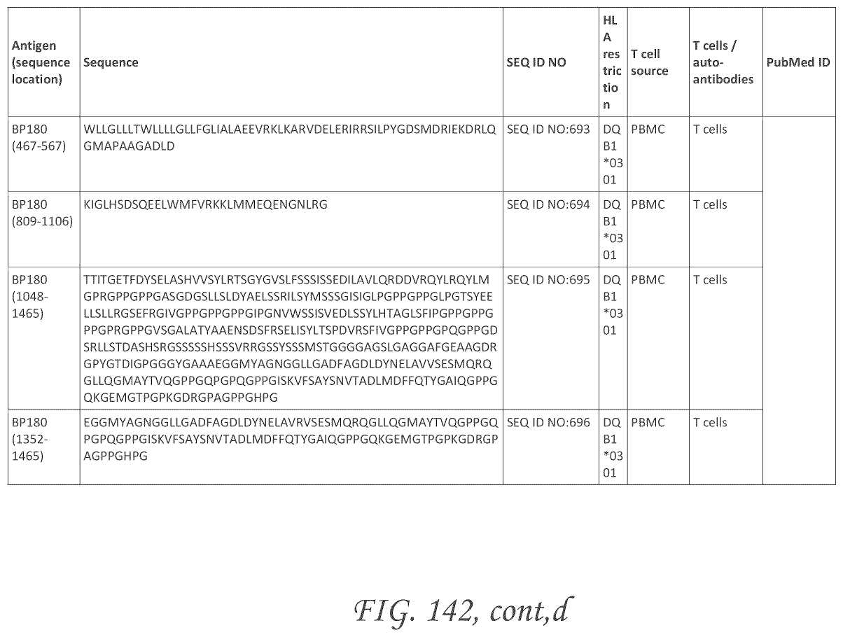

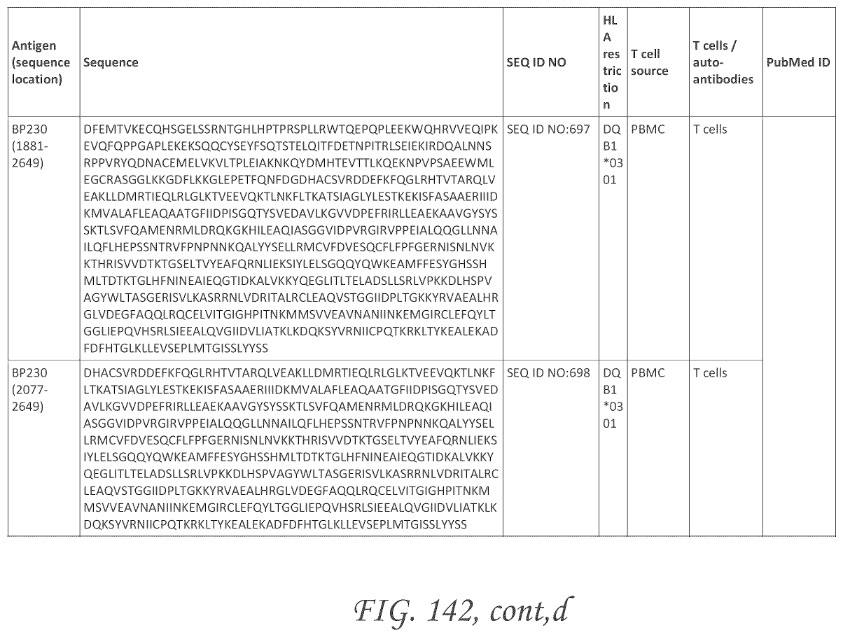

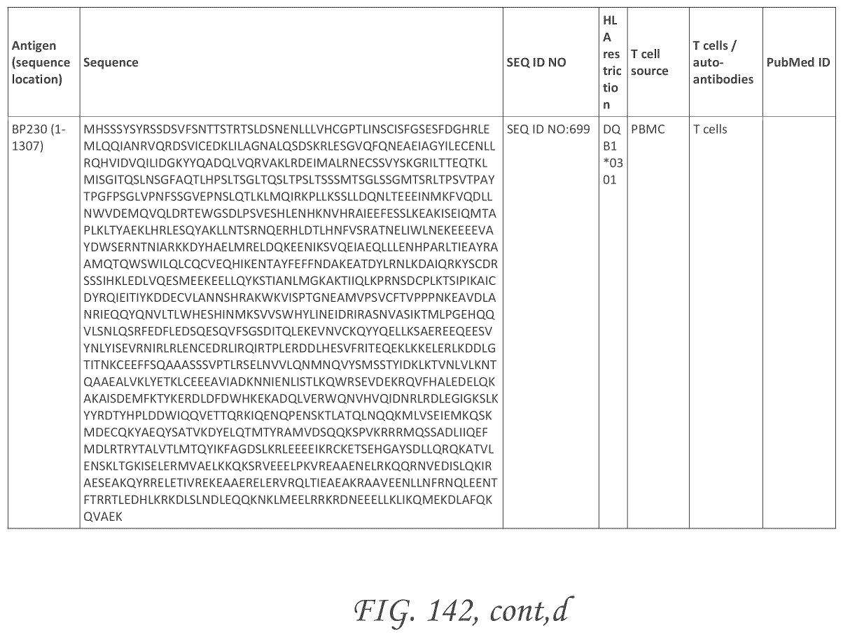

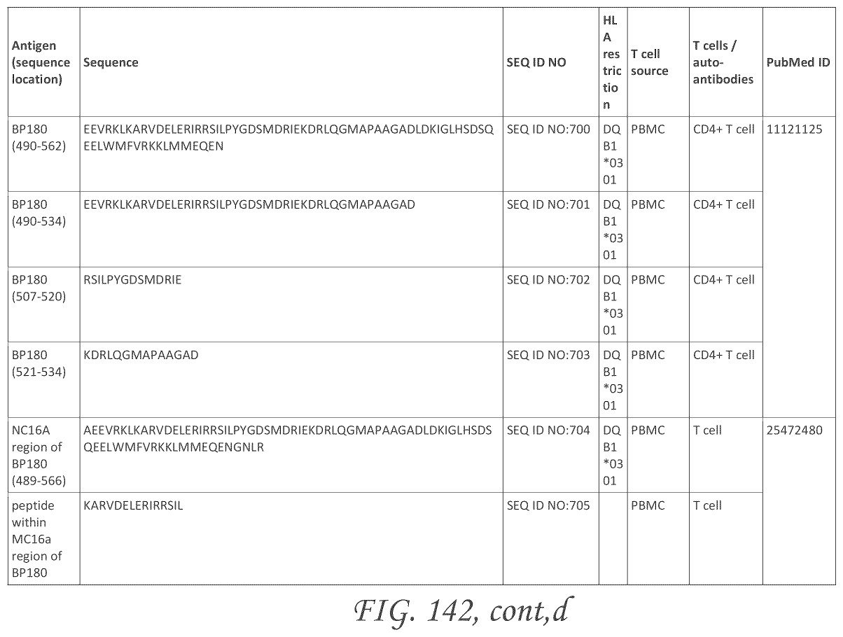

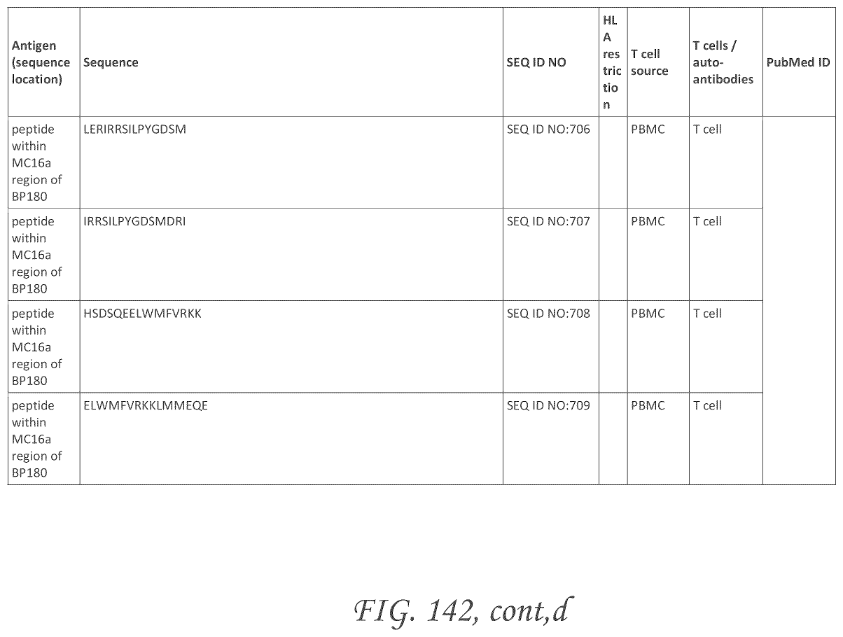

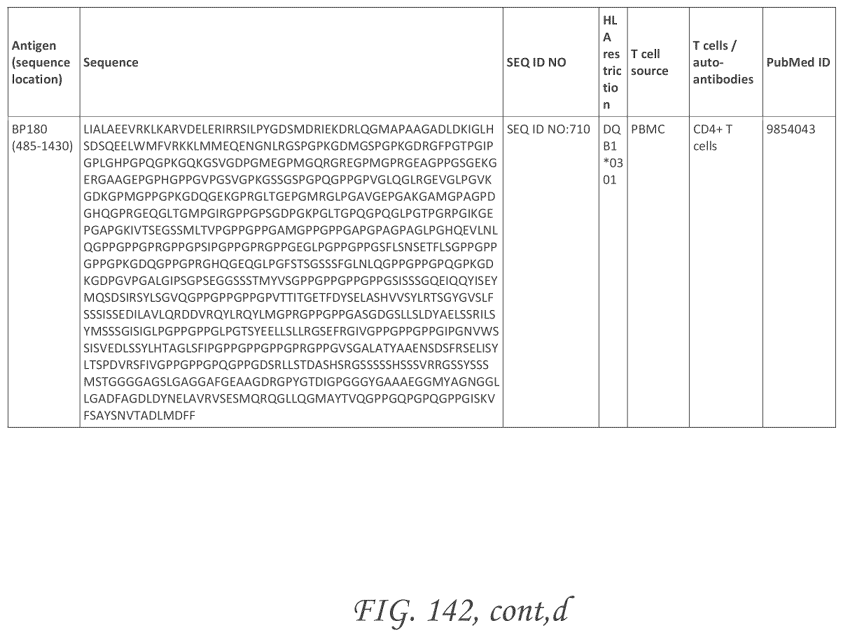

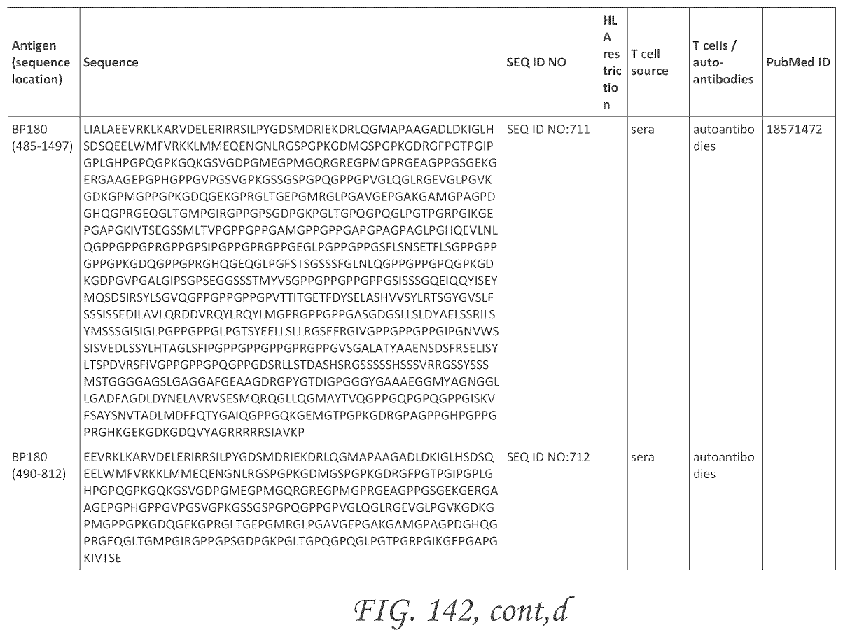

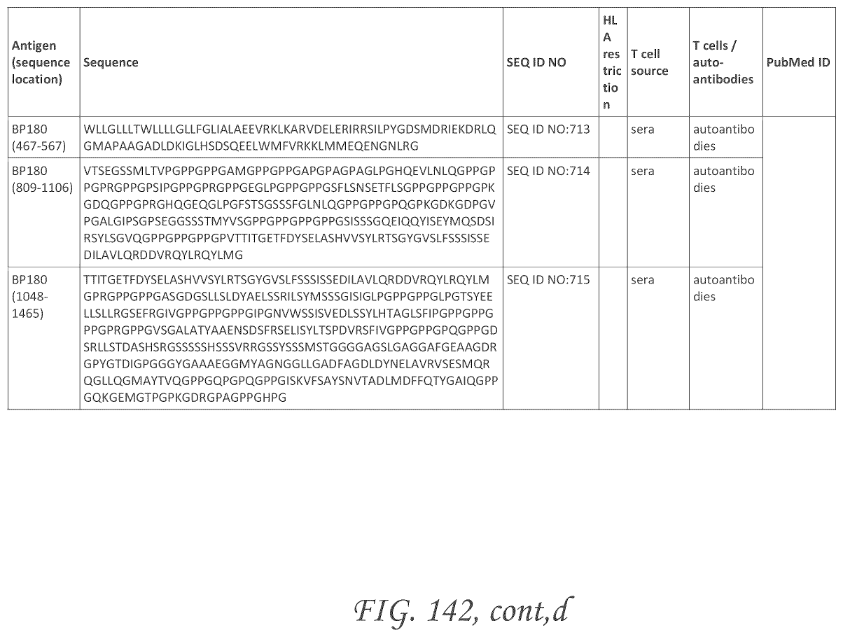

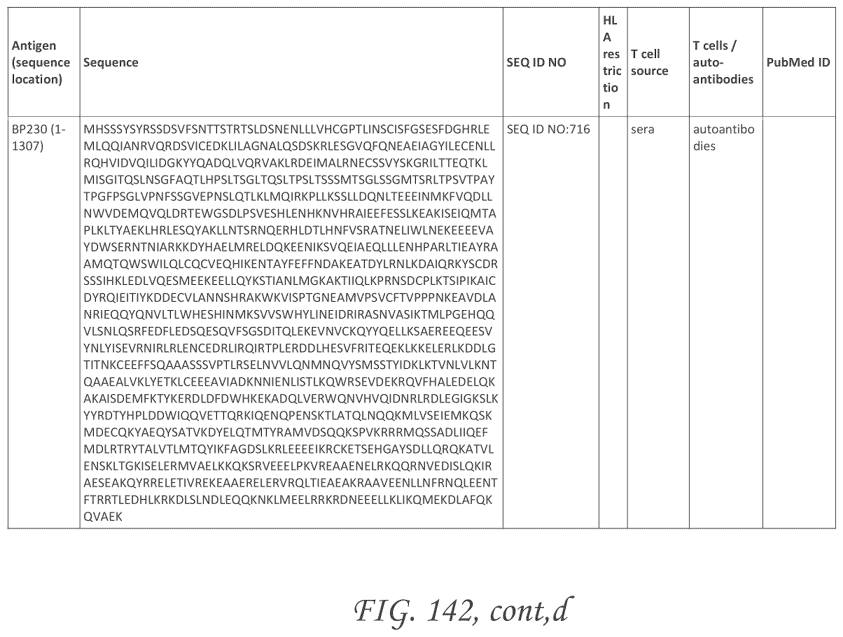

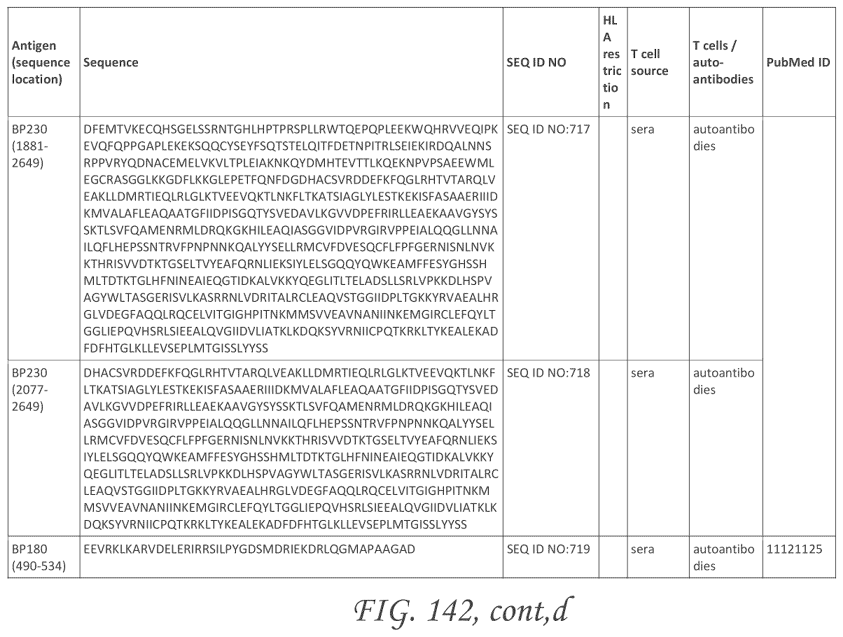

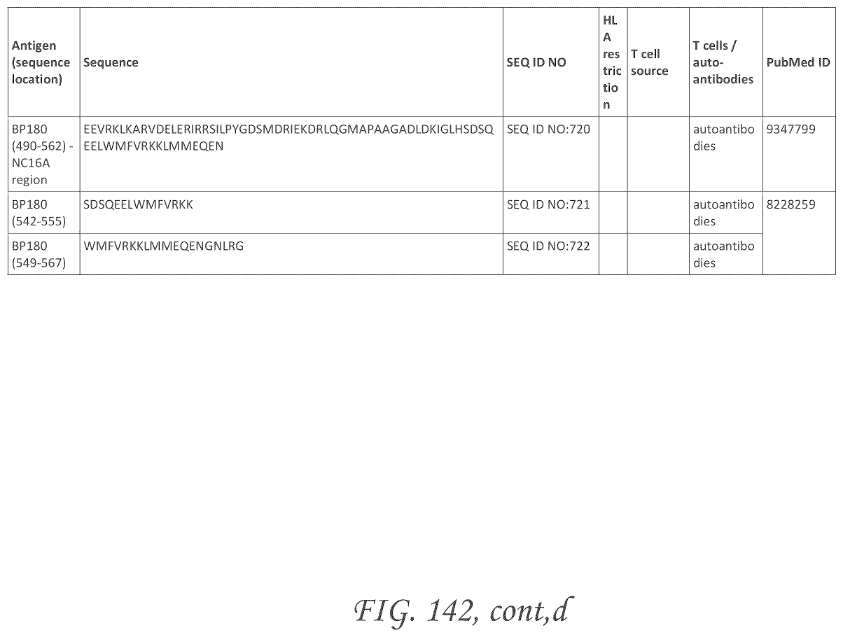

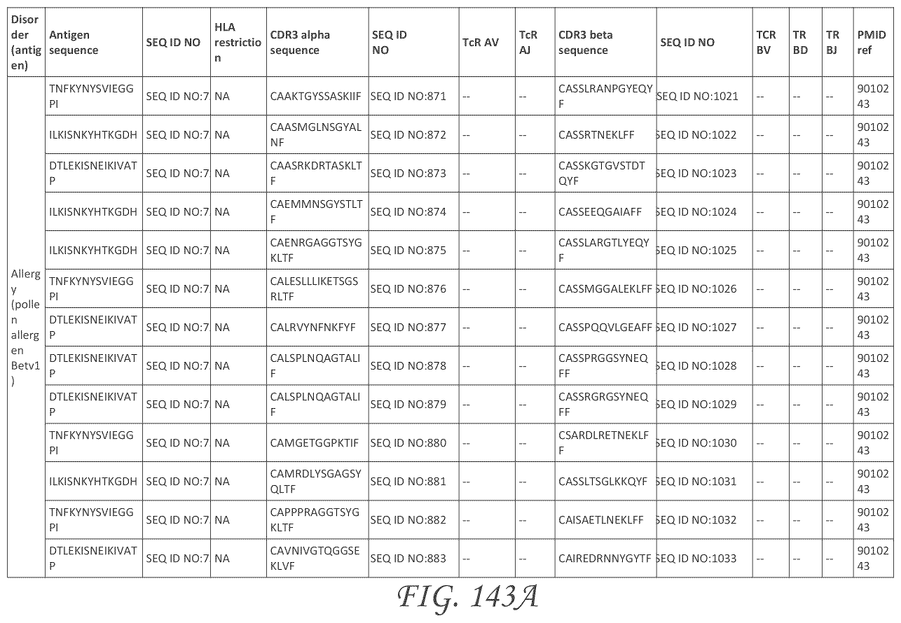

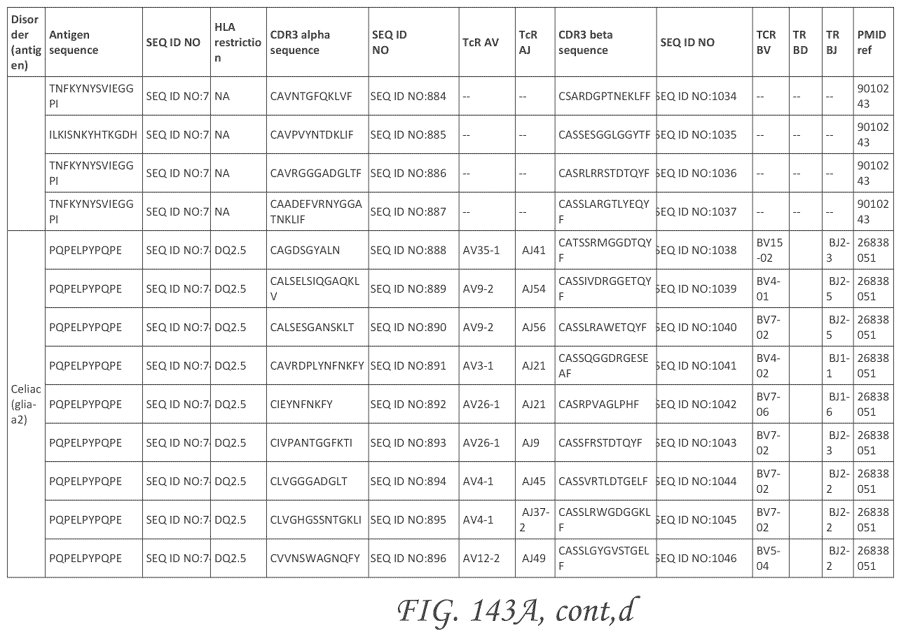

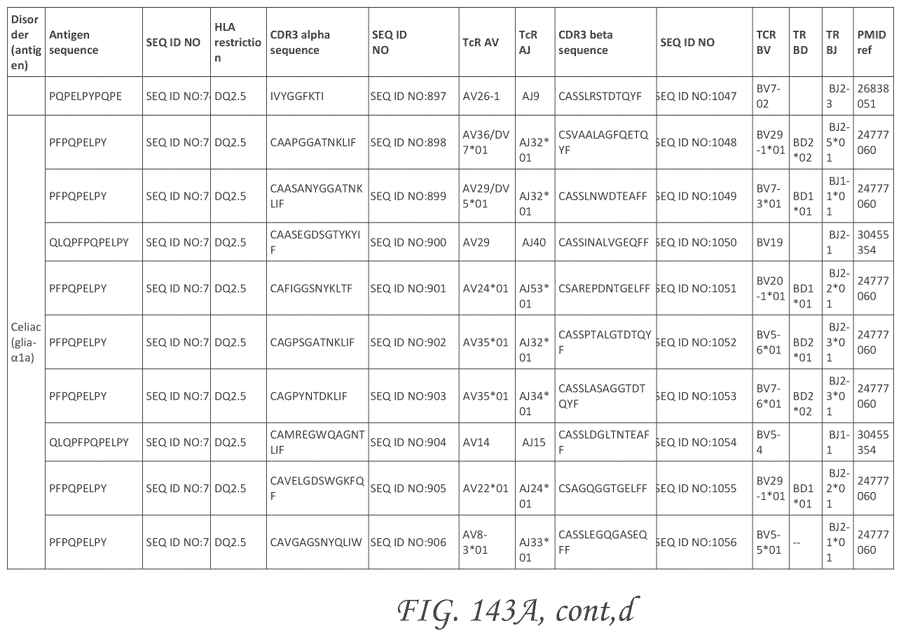

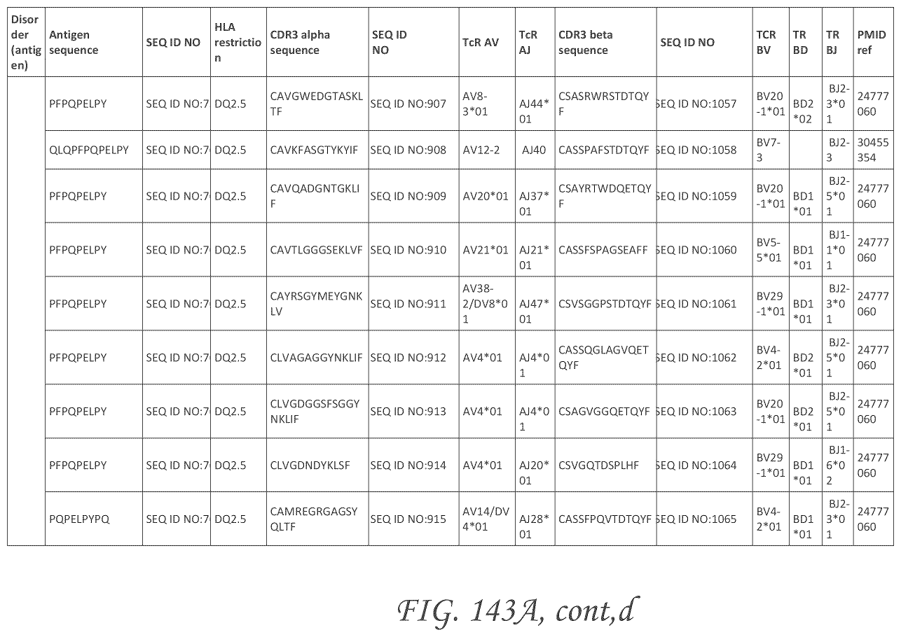

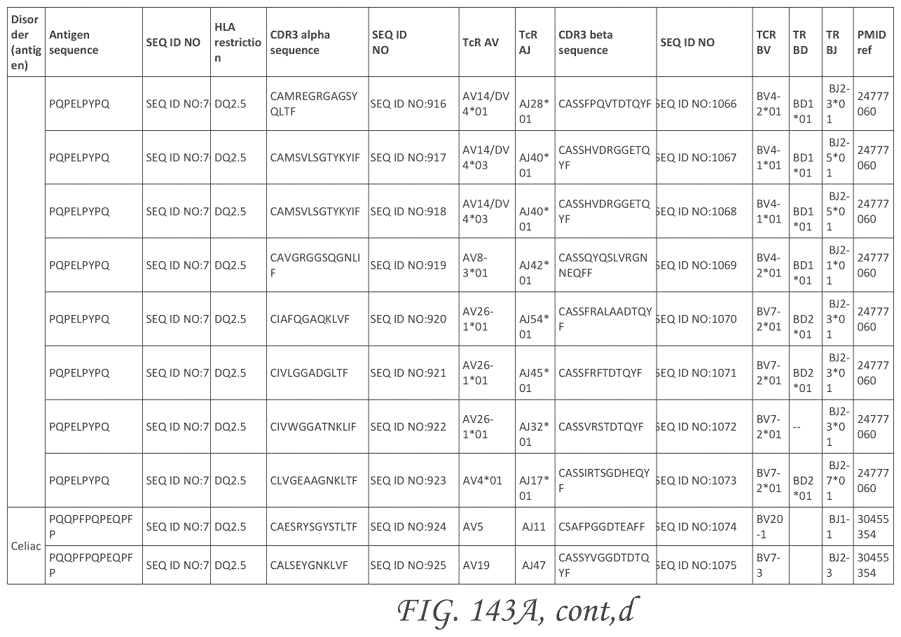

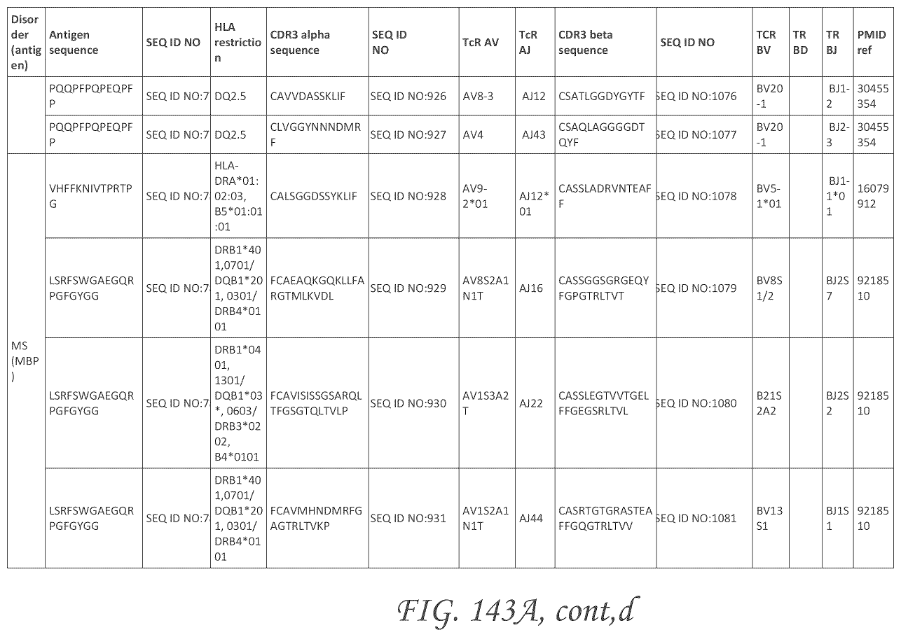

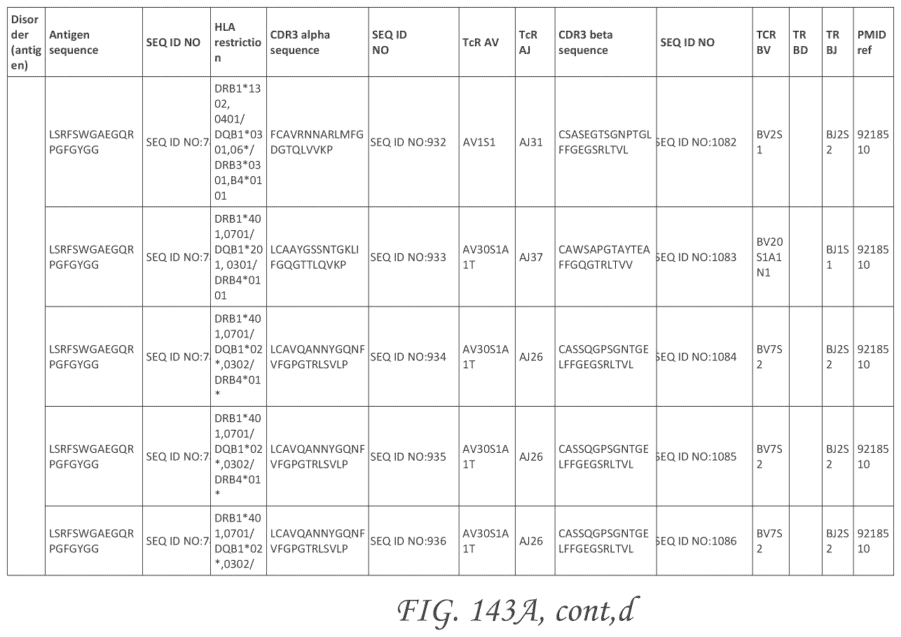

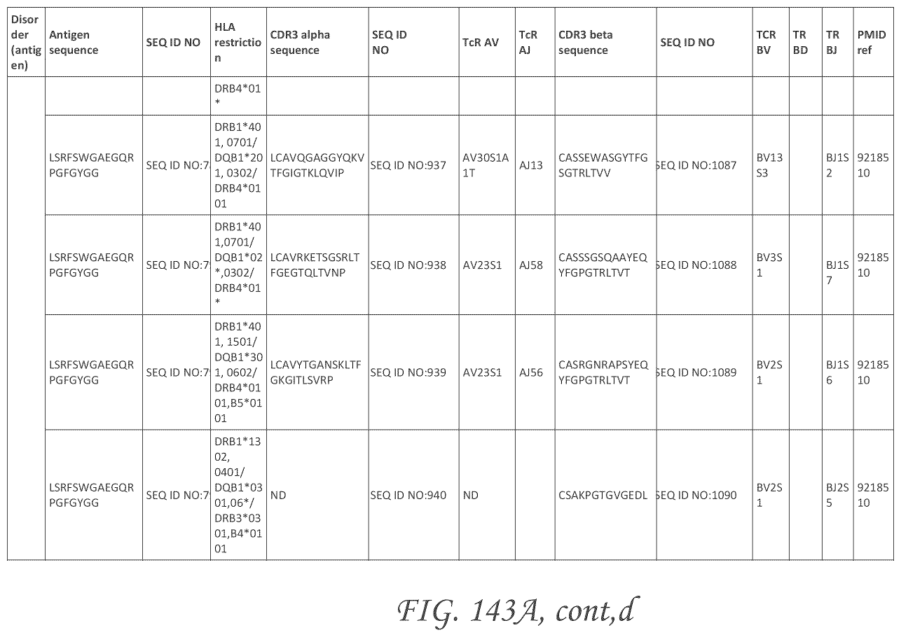

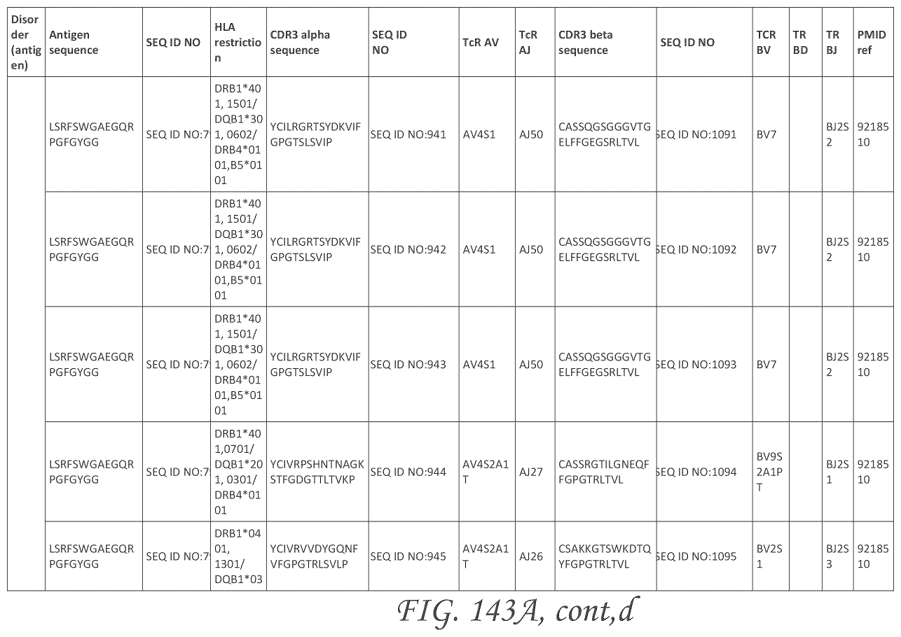

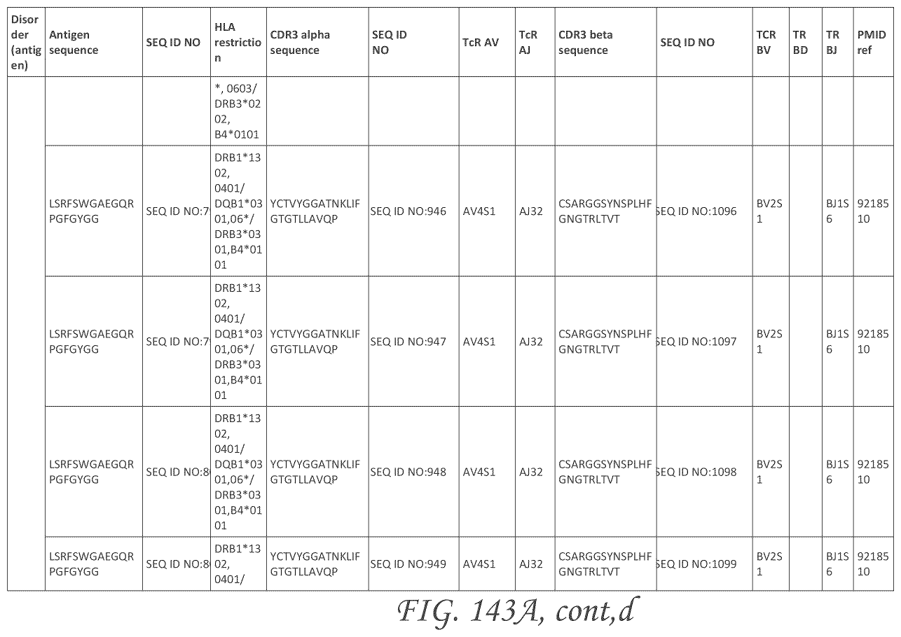

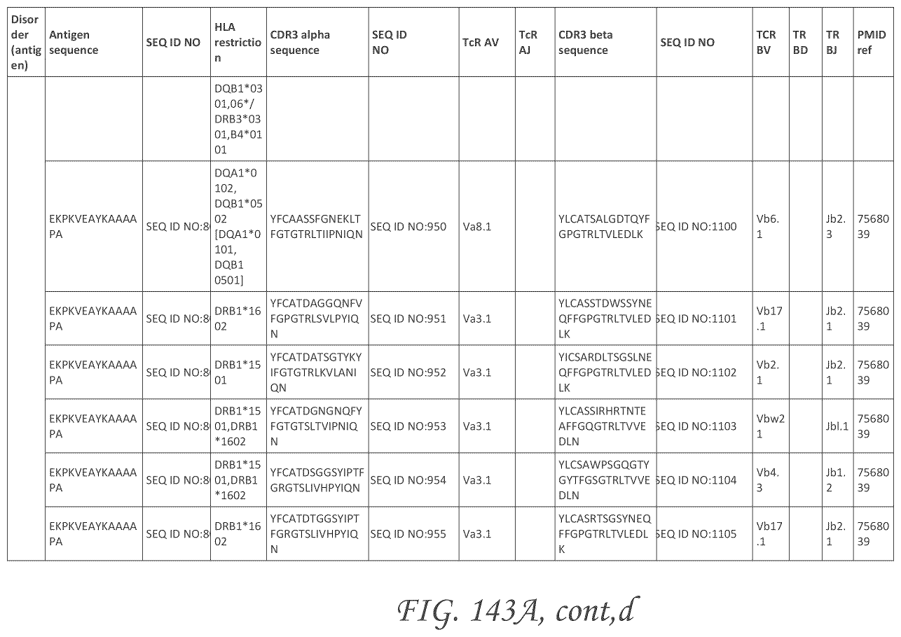

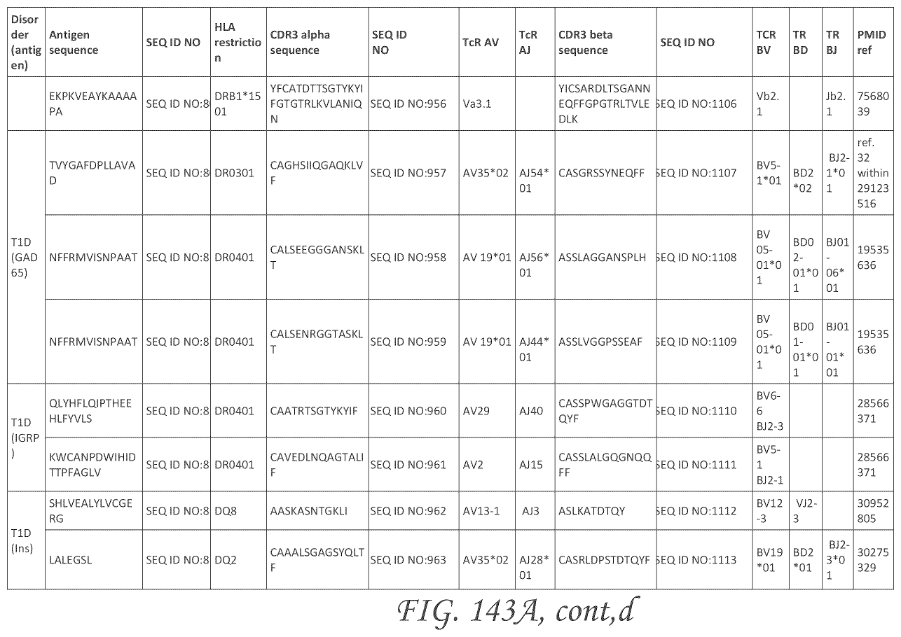

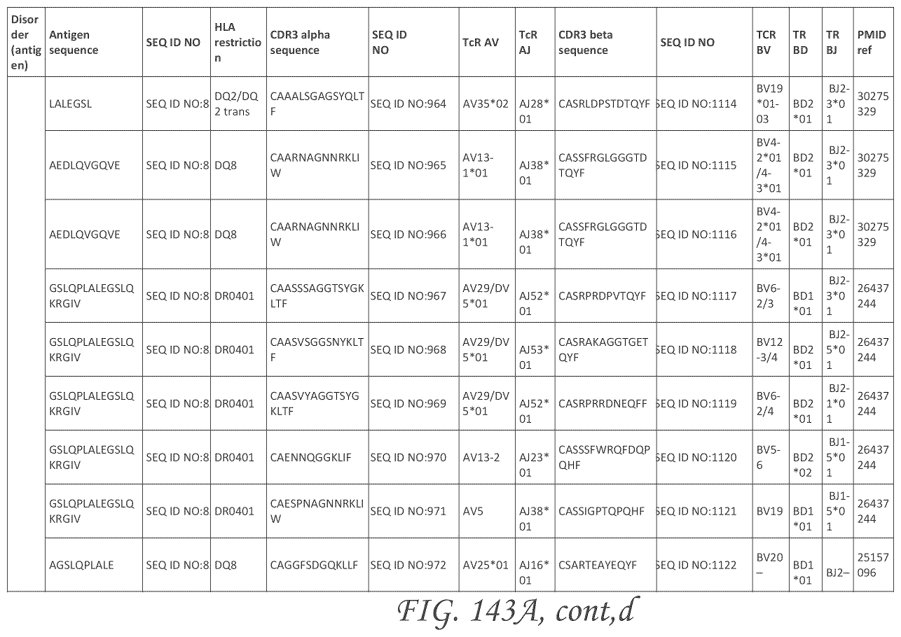

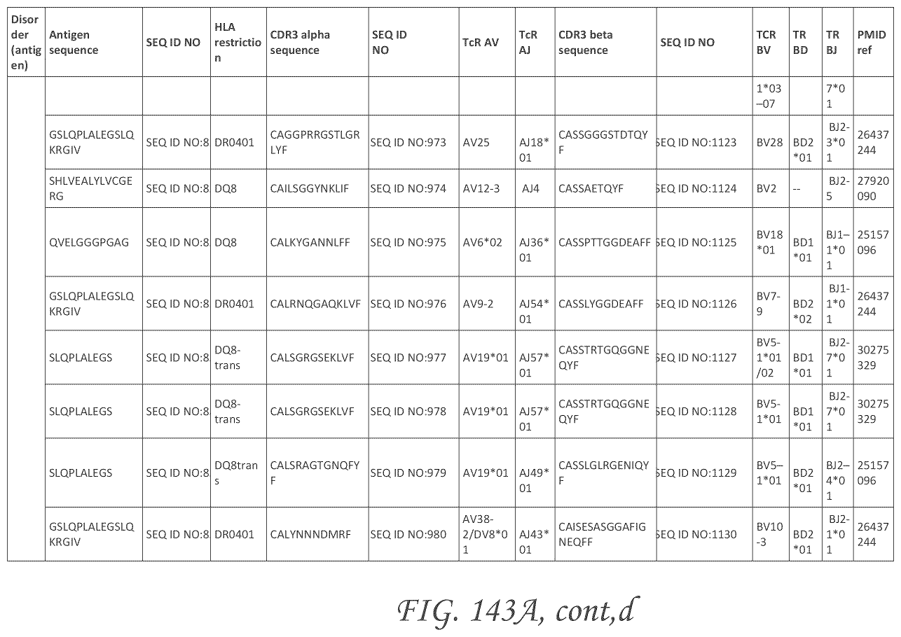

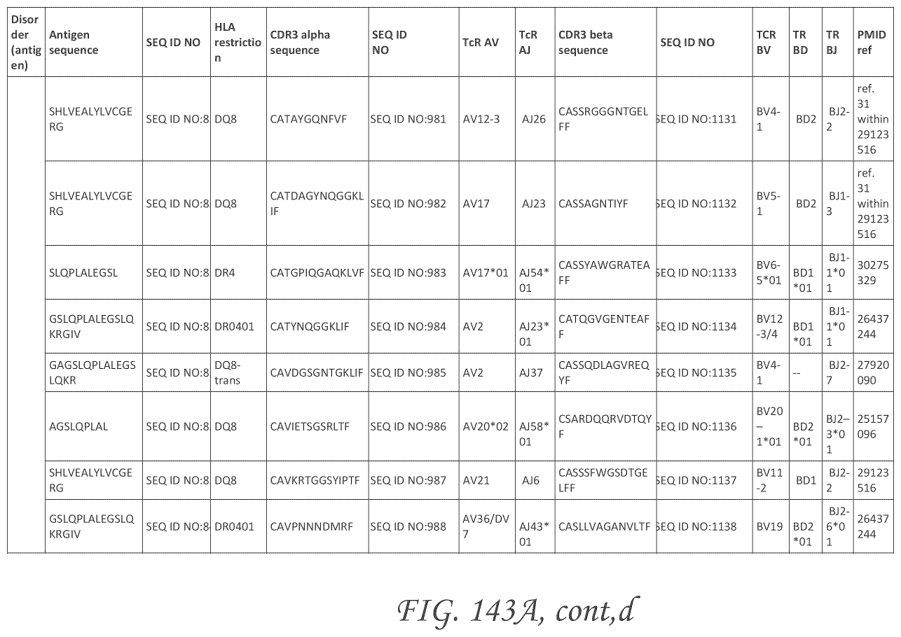

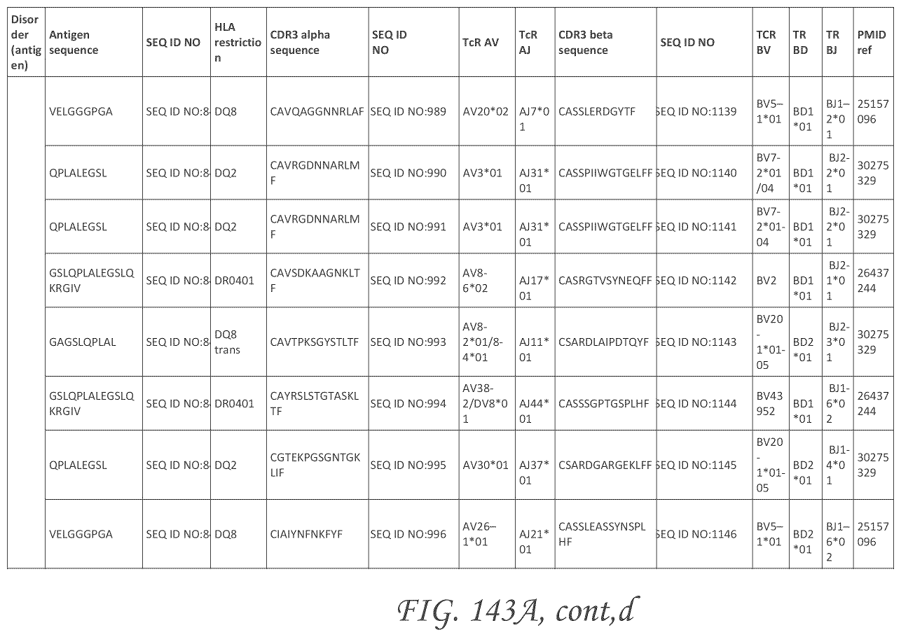

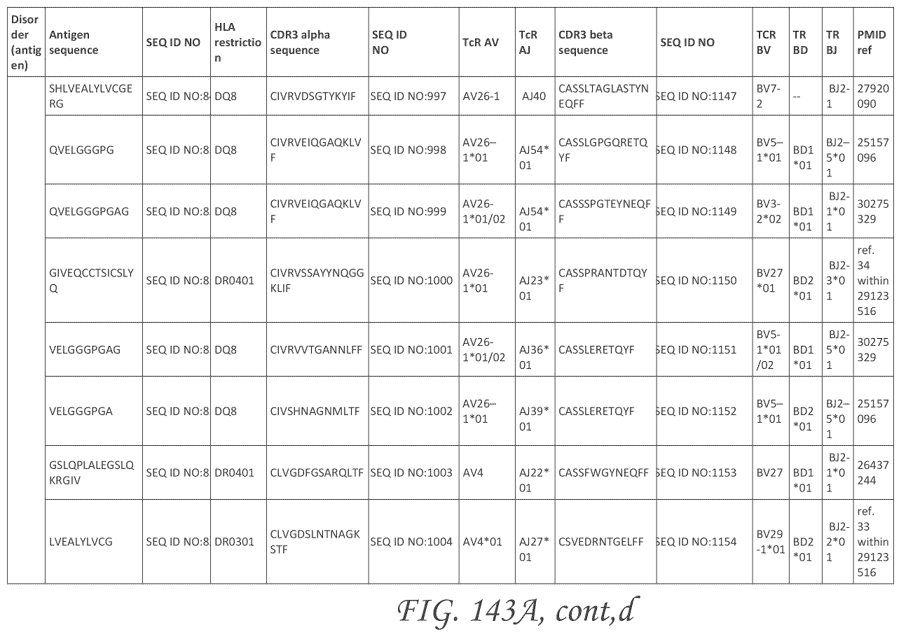

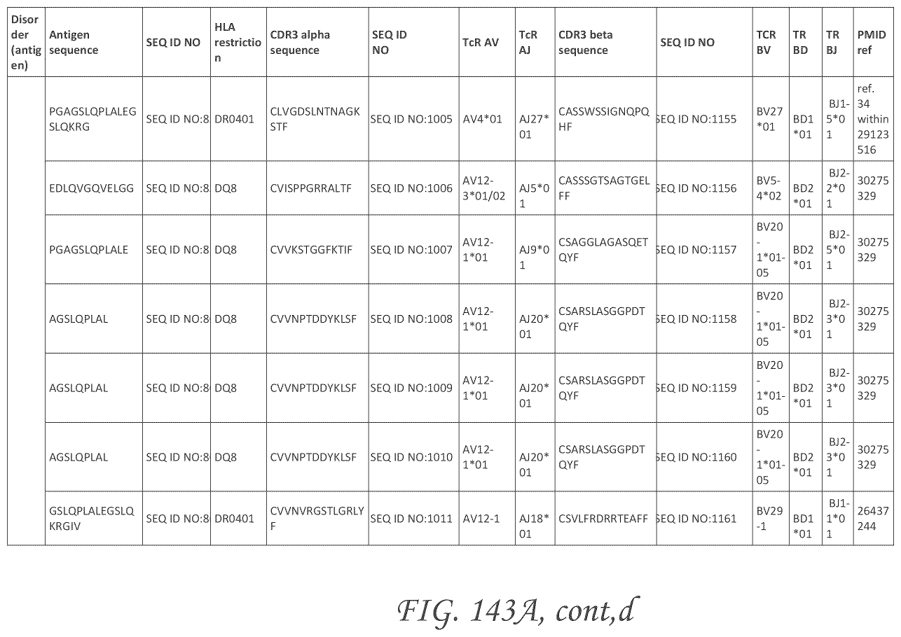

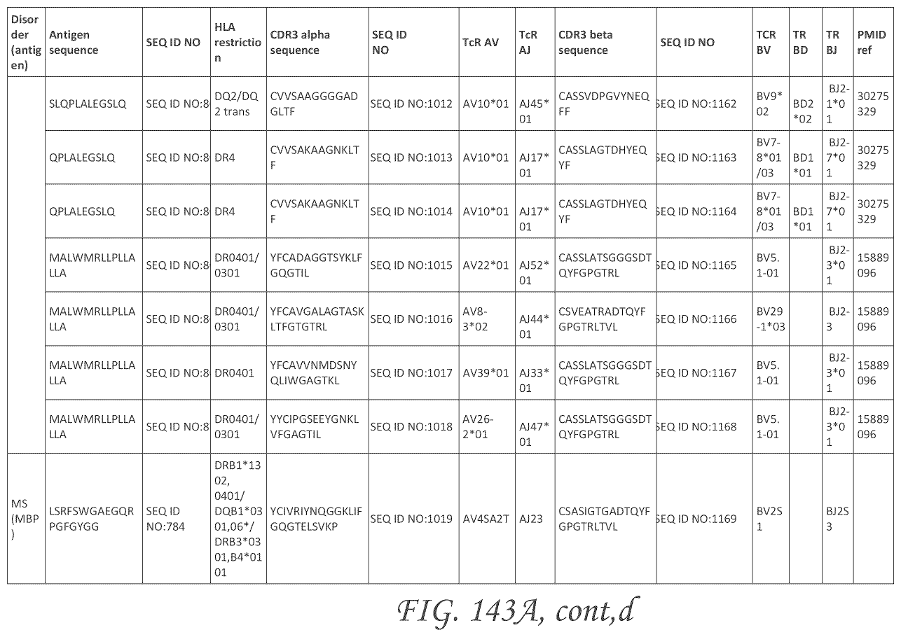

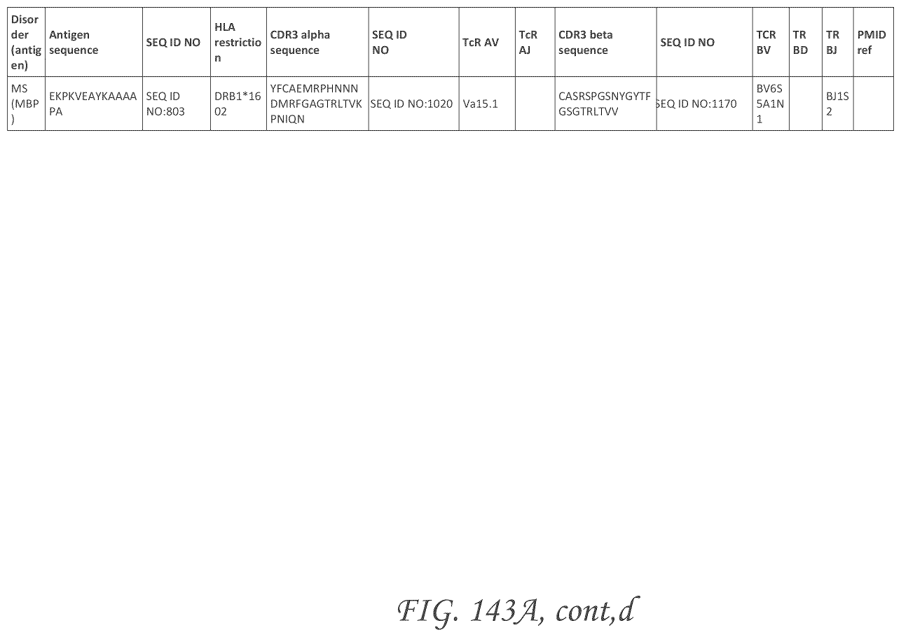

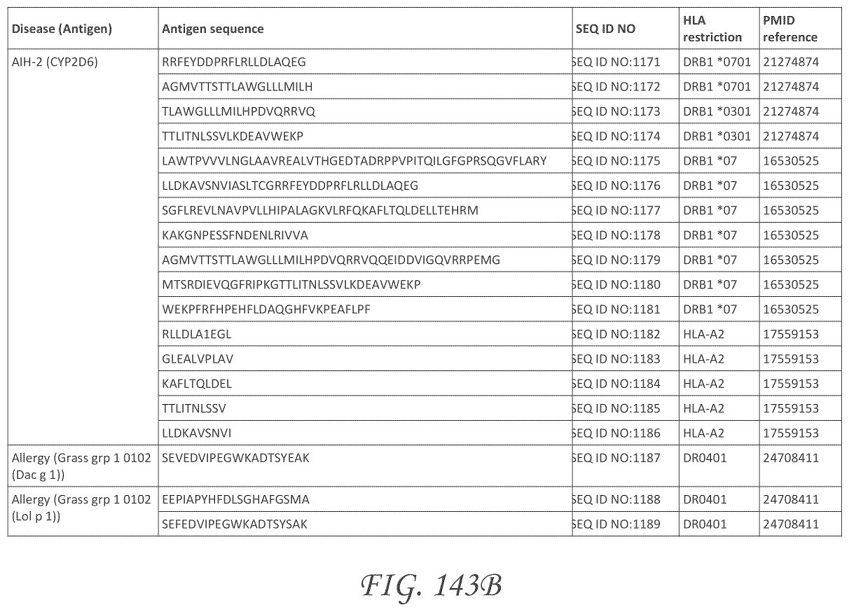

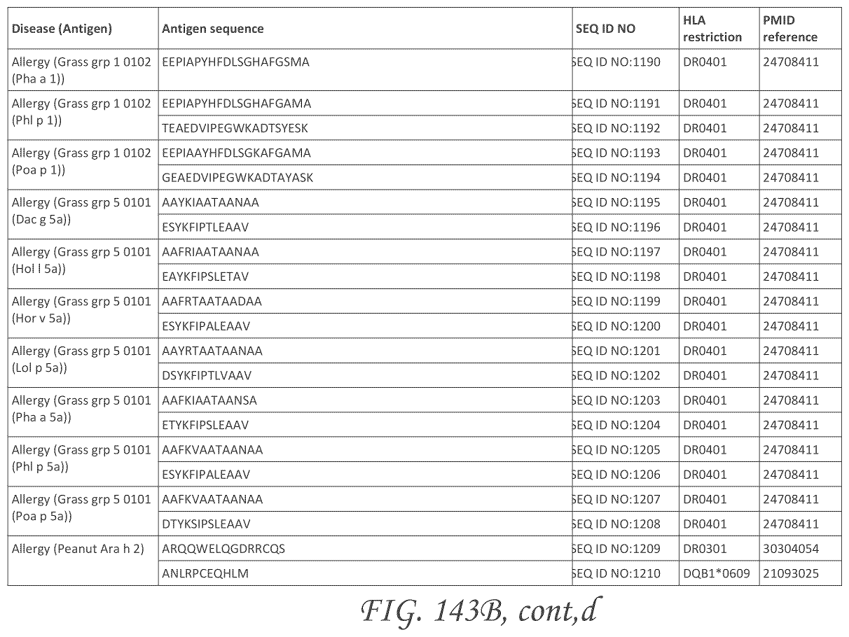

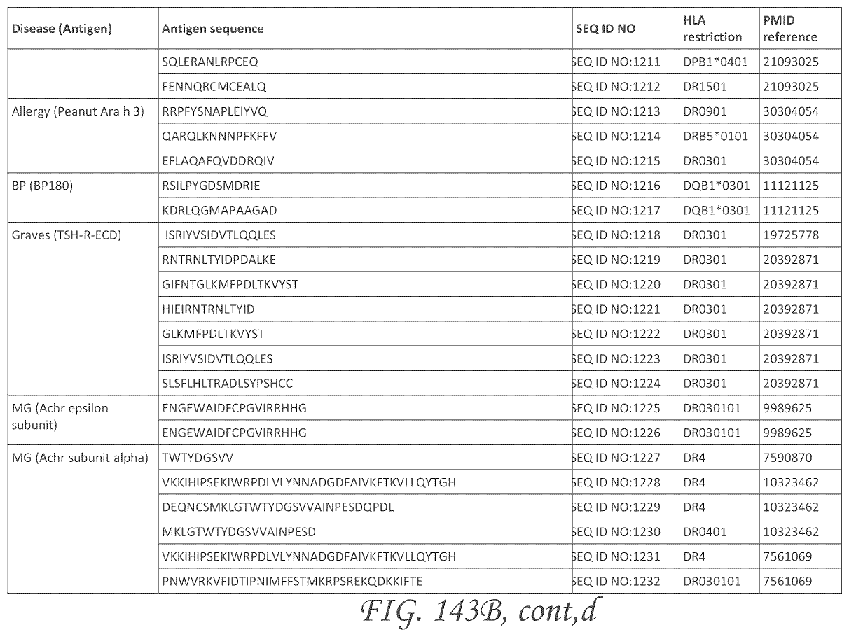

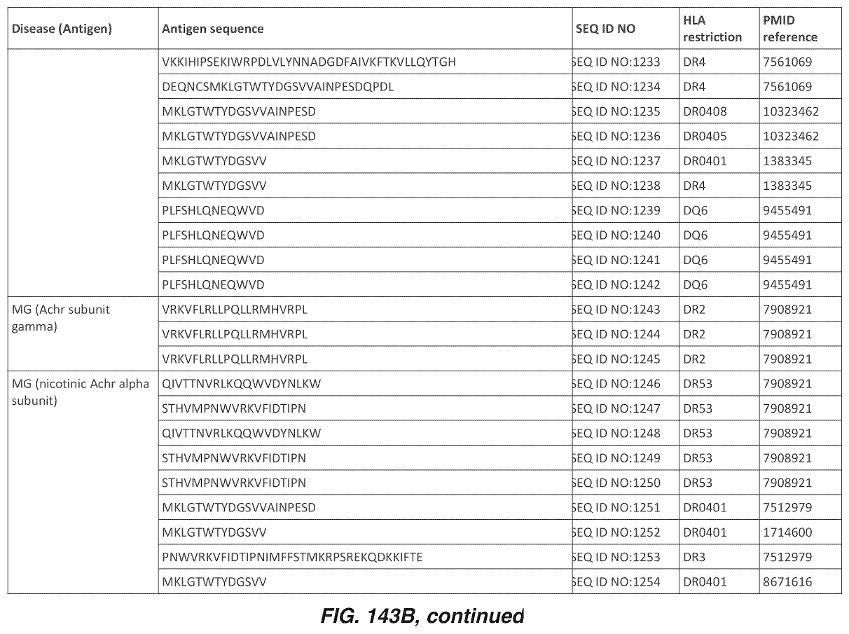

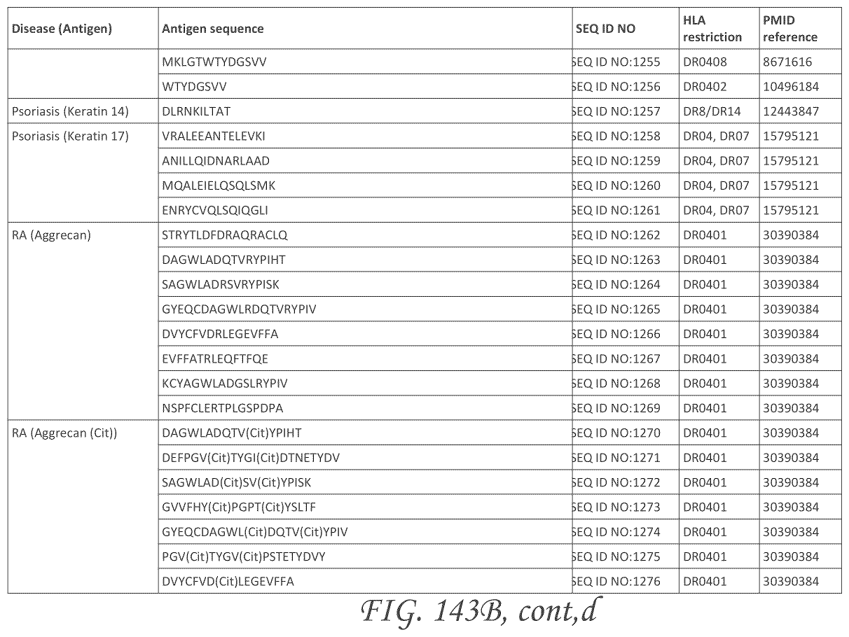

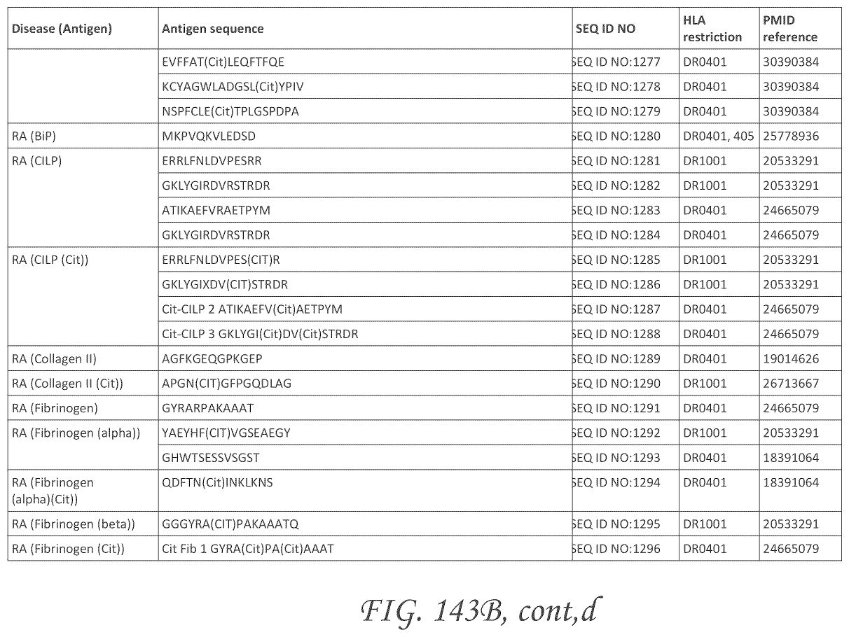

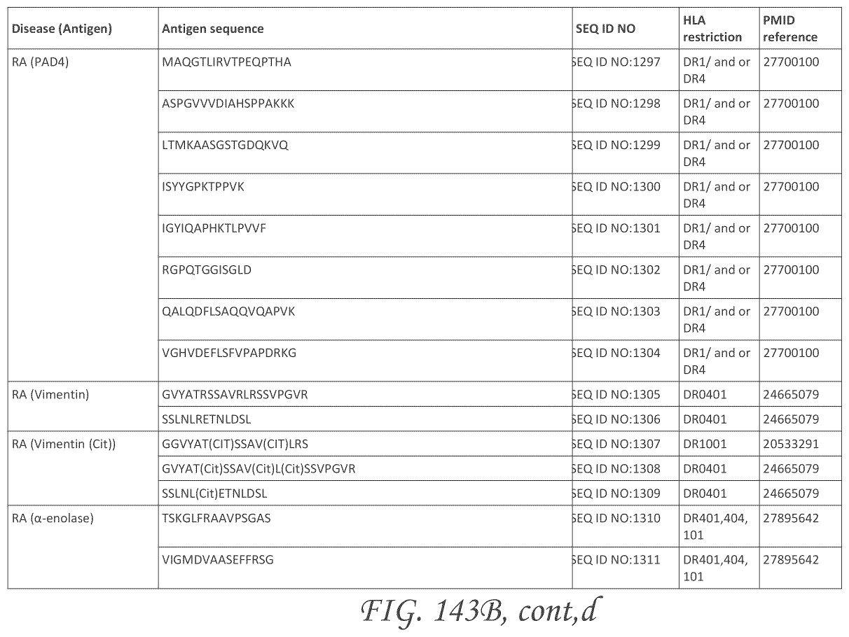

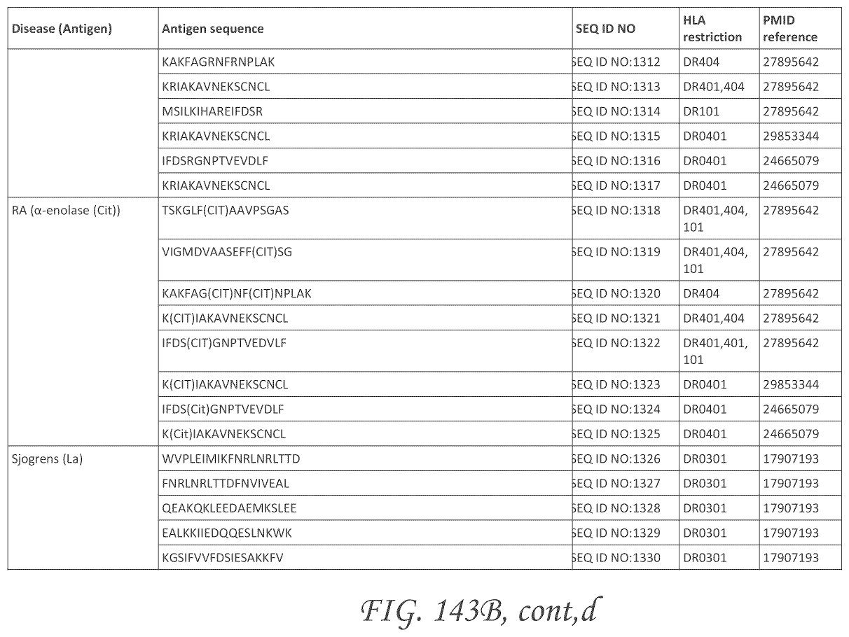

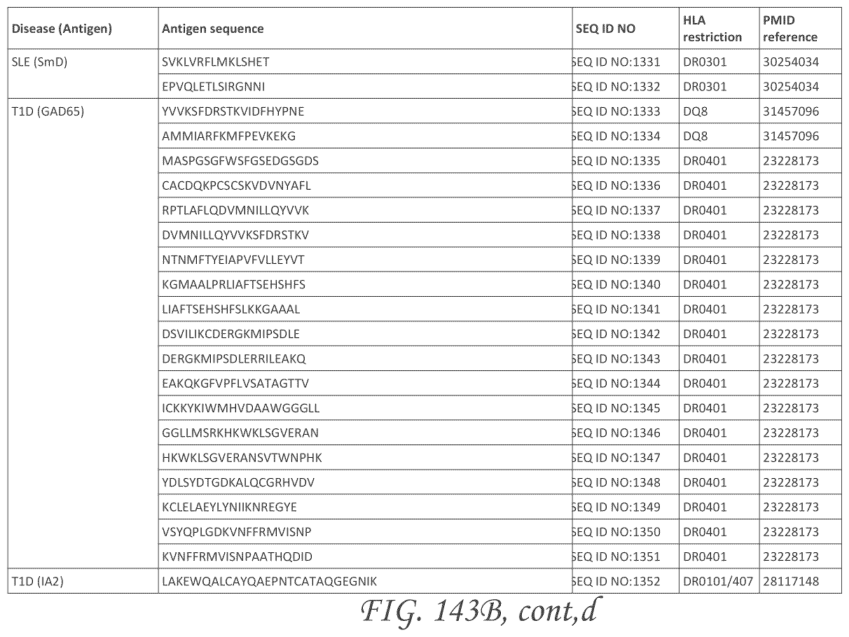

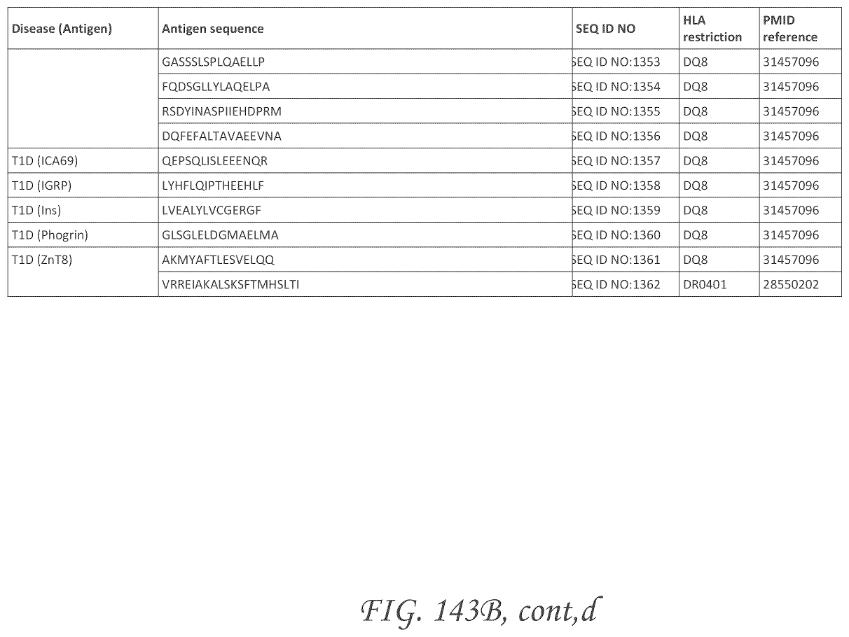

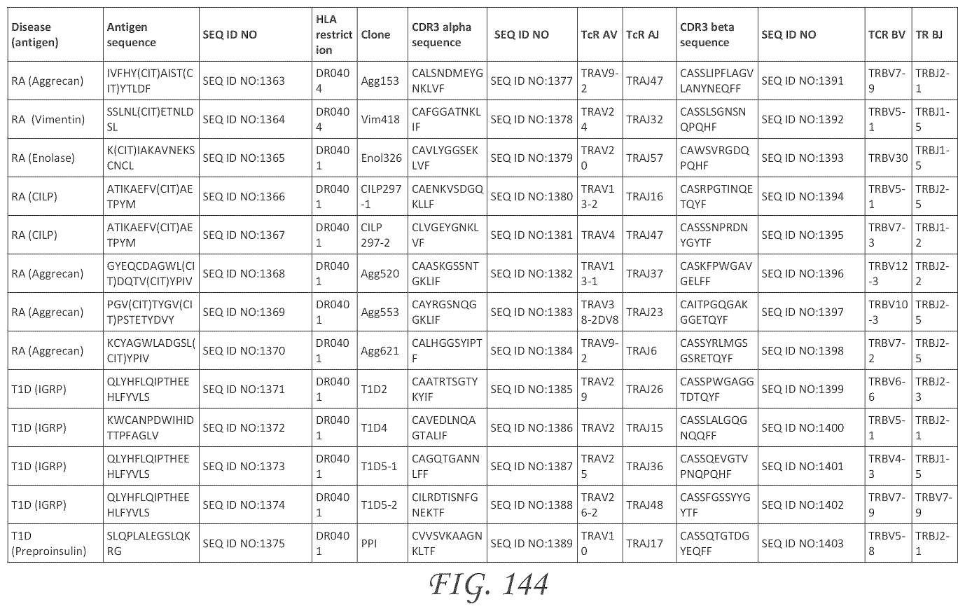

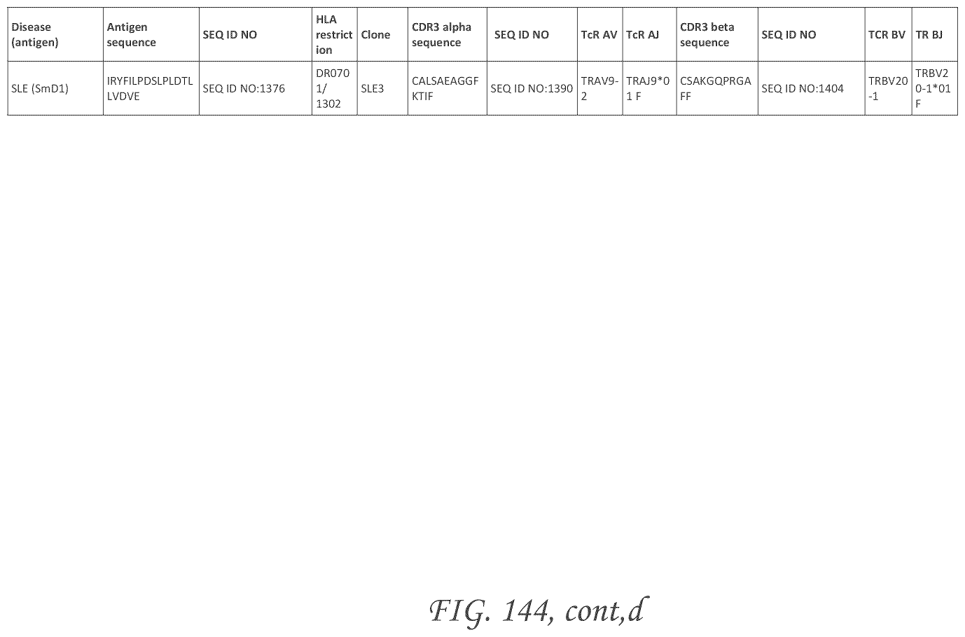

In another study, engineered BDC2.5+ antigen-specific (BDC) edTregs, or antigen-specific nTregs were infused into the mice followed by infusion of antigen-specific Teff cells. Mice were monitored for diabetes up to 33 days. FIG. 135 depicts a graph showing the percent of diabetic mice after receiving effector cells plus the designated mock edited, MND.LNGFR.P2A edited or nTreg cells from NOD BDC2.5 mice. Column enriched Ag-specific MND.LNGFR.P2A edTregs completely prevented diabetes in NSG mice and exhibited comparable function to nTregs. Strikingly, column-enriched LNGFR+ BDC2.5 edTregs completely prevented diabetes in NSG mice and exhibited comparable function to BDC2.5 nTreg in two separate experiments (FIG. 134 and FIG. 135).

### Example 14—Activity of a Truncated FRB-IL2RB Component in a Split CISC System

As disclosed herein, a split CISC system was used to edit two different genomic loci in cells, including antigen-specific engTreg cells. A split CISC system comprising a full-length FRB-IL2RB component was compared with a system comprising a truncated FRB-IL2RB component in which the IL2RB was truncated (See e.g., WO 2019/210057, which is expressly incorporated by reference in its entirety).

A dual editing strategy was employed using a FOXP3 knock-in construct (3324) encoding an FKBP-IL2RG component of the split-CISC system (FIG. 146, row A); and either a TRAC targeting HDR construct (3243) encoding a full-length FRB-IL2RB (FIG. 146, row B), or a TRAC targeting HDR construct (3333) encoding a truncated FRB-IL2RB (FIG. 146, row C). The FOXP3 knock-in construct also encoded an intracellular FRB polypeptide to bind intracellular rapalog. The TRAC targeting HDR constructs were designed to express a T1D4 islet TCR in combination with the FRB-IL2RB component of the split-CISC system.

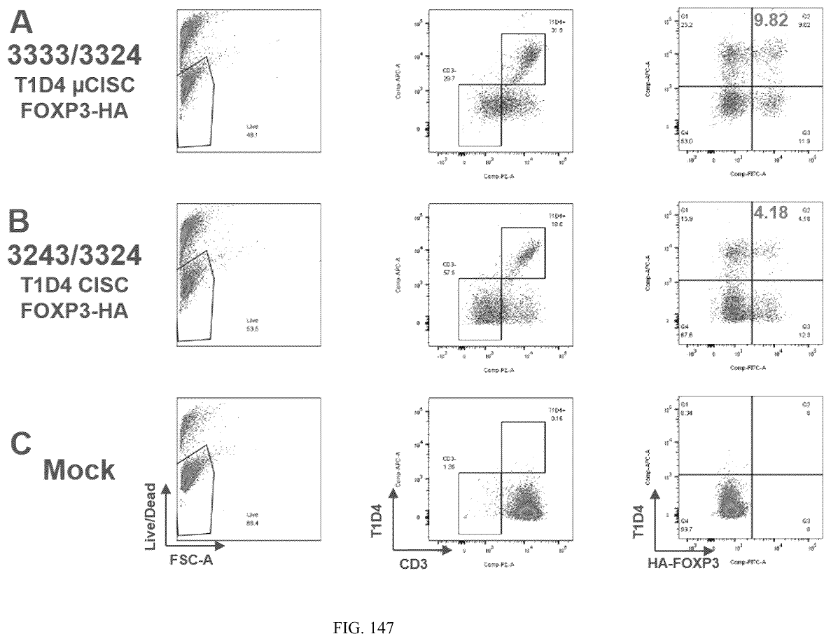

CD4+ T cells were transduced with either a combination of the 3324/3243 constructs or the 3324/3333 constructs. Three days after dual knock-in editing of CD4+ T cells, the inventors used FACS analysis to monitor protein expression and cell viability. Specifically, a FACS analysis was performed on the transduced cells for viability versus cell size (FSC-A), T1D4 expression versus CD3 expression, and T1D4 expression versus HA-tagged FOXP3 (FIG. 147).

Cells transduced with either a combination of the 3324/3243 constructs or the 3324/3333 constructs showed no significant decrease in viability compared to control “mock” cells (FIG. 147). However, there was a significant reduction of CD3 in cells containing a construct, as expected following TRAC targeting. Furthermore, cells expressing either construct had a significantly higher population of TID4/FOXP3-HA double positive cells compared with control. This demonstrated that both the FOXP3 construct (3324) and either the construct encoding a full-length FRB-IL2RB (3243) or the construct encoding a truncated FRB-IL2RB (3333) were expressed efficiently in CD4+ T cells.

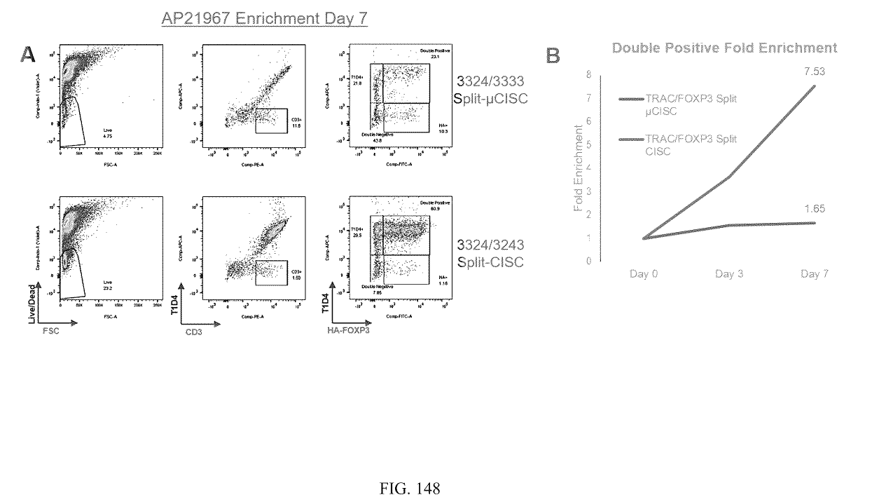

Transduced CD4+ T cells were contacted for 7 days with 100 nM AP21967 (Rapalog, a rapamycin derivative). At the end of 7 days, the cells underwent FACS analysis for viability versus cell size (FSC-A), T1D4 expression versus CD3 expression, and T1D4 expression versus HA-tagged FOXP3 (FIG. 148). While there was no significant change in viability between cells expressing either the construct encoding a full-length FRB-IL2RB (3243) or the construct encoding a truncated FRB-IL2RB (3333), cells transduced with the truncated FRB-IL2RB (3333) construct had significantly lower signal for T1D4 and FOXP3-HA compared to cells transduced with the full length FRB-IL2RB (3243) construct. Parallel quantification of double positive fold enrichment indicated that cells with the 3243 construct had approximately 4-times higher enrichment than cells with the 3333 construct. This surprising observation provided evidence that expression levels of FKB-IL2RG were likely rate limiting for CISC functional activity.

### Example 15—Effect of Orientation of an FRB-IL2RB Component in a Construct of a Split CISC System

The effect of the orientation of an FRB-IL2RB component in a construct of a split CISC system was tested. A construct comprising a TRAC targeting construct (3323) in which sequences encoding a truncated FRB-IL2RB were proximal to an MND promoter was prepared (FIG. 149, row A), and compared to a TRAC targeting construct (3333) in which sequences encoding the truncated FRB-IL2RB were distal to an MND promoter with intervening sequences encoding a T1D1 TCR polypeptide (FIG. 149, row B).

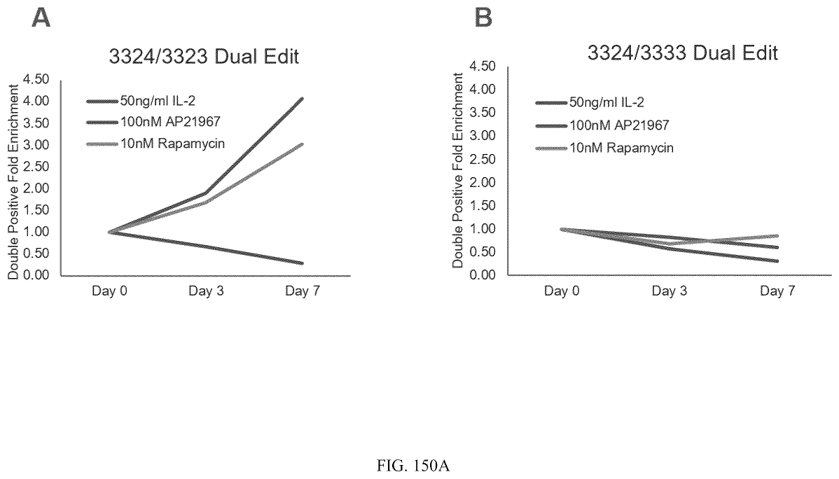

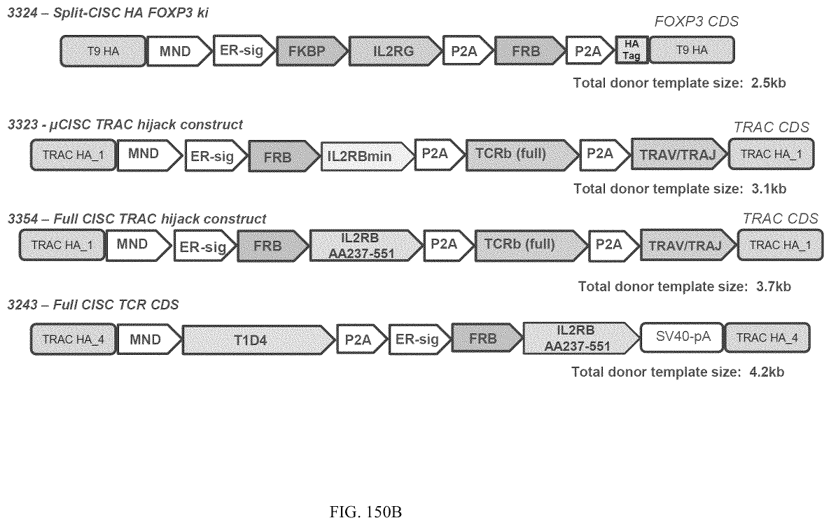

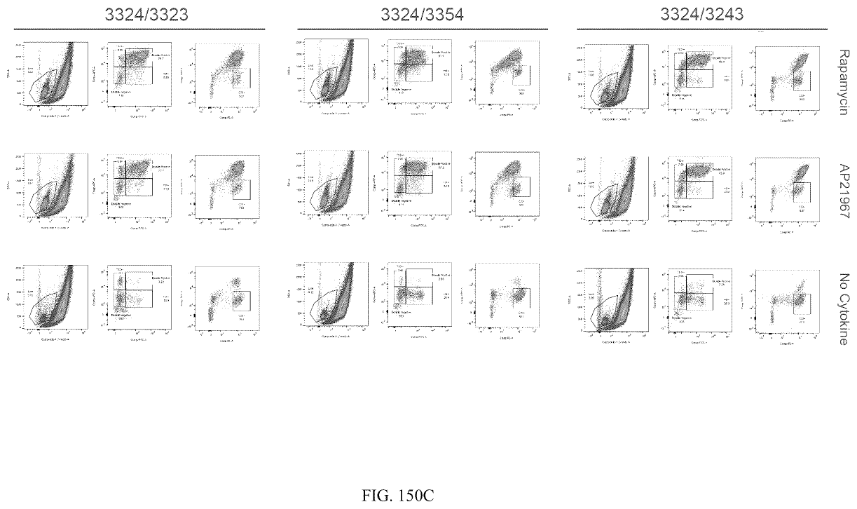

CD4+ T cells were transduced with a combination of a FOX3P targeting construct (3324) and either the 3323 construct or the 3333 construct. The enrichment capacity for the alternative TRAC targeting constructs was compared in a 7-day time course using 5 ng/mL IL-2, 100 nM AP21967 (Rapalog), or 10 nM Rapamycin (FIG. 150A). Unexpectedly, cells transduced with 3324/3323 had significantly greater enrichment for T1D4 and FOXP3-HA compared to cells transduced with 3324/3333 (FIG. 150A). This finding demonstrated that repositioning the sequences encoding the truncated FRB-IL2RB to be proximal to the MND promoter markedly improved the split-CISC system functionality.

An additional example related to a comparison of split CISC enrichment is depicted in FIG. 150B, FIG. 150C, and FIG. 150D. Constructs comprising sequences encoding a truncated FRB-IL2RB proximal to an MND promoter, (3323 and 3354), and a construct in which sequences encoding the truncated FRB-IL2RB were distal to an MND promoter with intervening sequences encoding a T1D4 TCR polypeptide (3234) were prepared (FIG. 150B). CD4+ T cells were transduced with a combination of a FOX3P targeting construct (3324) and either the 3323, 3354 or 3243 construct. The enrichment capacity for the alternative TRAC targeting constructs was compared in an 8-day time course using 100 nM AP21967 (Rapalog), or 10 nM Rapamycin. FIG. 150C depicts a FACS analysis of transduced cells. FIG. 150D depicts graphs of absolute vs fold enrichment between the different dual editing groups.

### Example 16—Effect of Promoter Linked to an FRB-IL2RB Component in a Construct of a Split CISC System

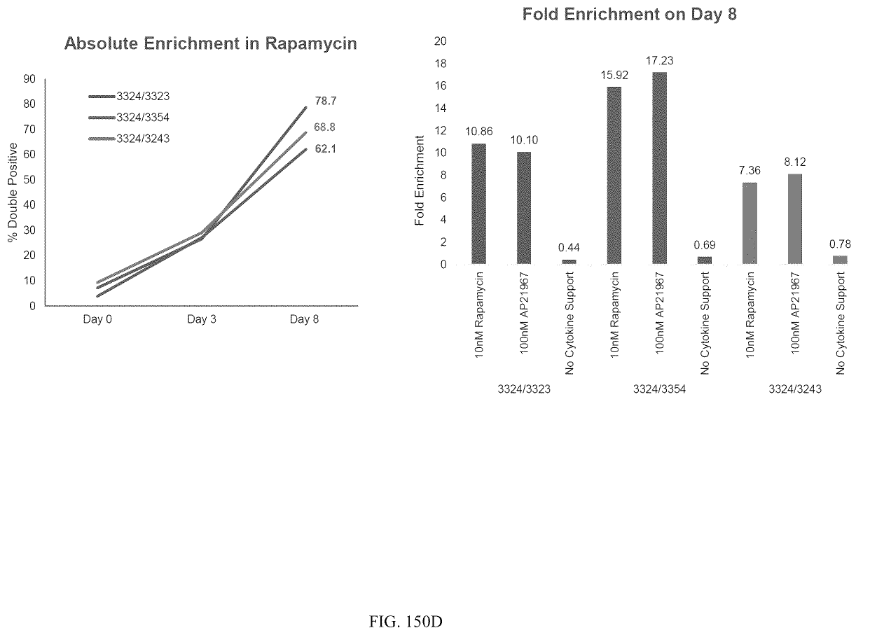

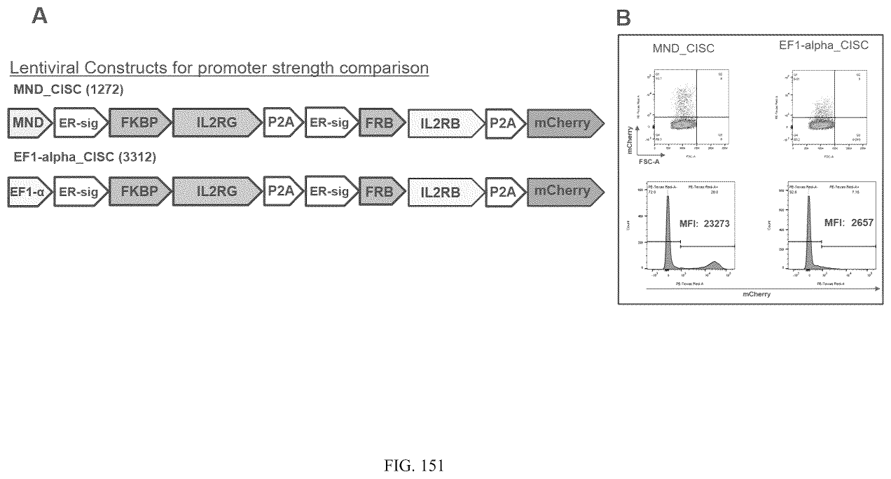

The finding that split CISC function was dependent upon location of elements within the HDR donor cassette suggested that CISC function may be dependent upon protein expression levels. To test this idea, lentiviral vectors were generated containing alternative promoters designed to express the CISC cassette at alternative levels. The constructs contained either the MND promoter linked to sequences encoding the FRB-IL2RB (1272), or the EF1-alpha promoter linked to sequences encoding the FRB-IL2RB (3312), each construct also included sequences encoding an mCherry reporter gene (FIG. 151, panel A). CD4+ T cells were transduced with either construct.

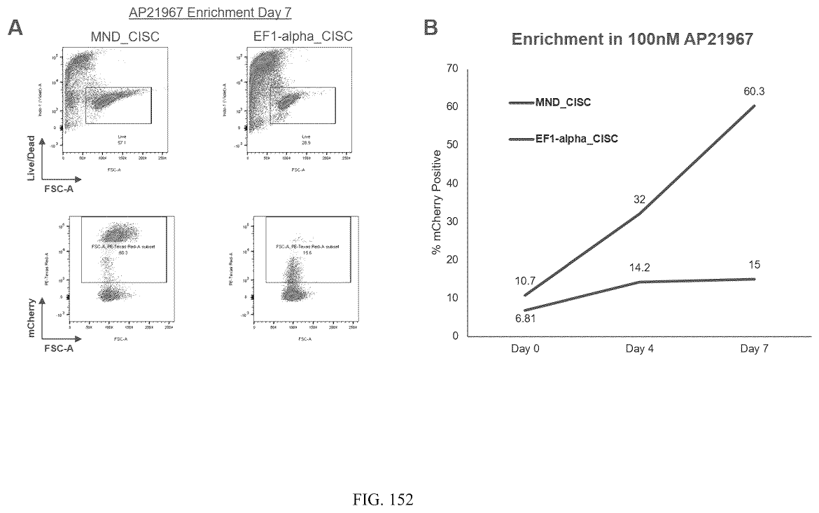

As demonstrated by flow cytometry analysis of mCherry expression per cell, the EF1a promoter resulted in significantly lower overall expression in CD4+ T cells (FIG. 151, panel B). Following 7 days exposure to 100 nM AP21967, cells with the EF1a promoter showed significantly weaker expression as analyzed by FACS (FIG. 152, panel A). Subsequent graphing of the fold enrichment for mCherry expression indicated that the EF1a promoter induced 4-fold lower expression than the MND promoter (FIG. 152, panel B). Consequently, lower expression levels reduced CISC function. Overall, this data provided evidence that a threshold level of expression of split CISC components may have a role in the overall activity of the split CISC system.

### Example 17—In Vitro Suppression Assay with edTreg Cells

The activity of islet-specific edTregs to suppress islet-specific T cells was determined. Briefly, polyclonal islet-specific Teff cells, islet-specific edTreg cells, and monocyte-derived dendritic cells (mDC) were generated from peripheral blood mononuclear cells (PBMC) from T1D or control donors for use in a suppression assay (FIG. 154, panel A).

The polyclonal islet-specific Teff cells were generated by isolation of T cells (CD4+CD25−) from PBMCs, incubation with irradiated autologous antigen-presenting cells (APC) (CD4−CD25+) and a pool of islet-specific peptides (GAD65₁₁₃₋₁₃₂, GAD65₂₆₅₋₂₈₄, GAD65₂₇₃₋₂₉₂, GAD65₃₀₅₋₃₂₄, GAD65₅₅₃₋₅₇₂, IGRP₁₇₋₃₆, IGRP₂₄₁₋₂₆₀, IGRP₃₀₅₋₃₂₄, and PPI₇₆₋₉₀) at 5 μg/ml for a total of 12-14 days (FIG. 153, panel A). After 7 days of incubation, cells were expanded in media with IL-2 at 20 ng/mL added in 2-3-day intervals. Cells were harvested between day 12-14 and islet-specific T cells were detected by tetramer staining (FIG. 153, panels B and C). The tetramer+ population was assessed and combined over five different experiments using 3 individual T1D donors after 12-14 of in vitro peptide stimulation. Staining with no tetramer was included as a negative staining result. A population of T cells enriched for a mixture of islet-specific T cells was obtained. A fraction of the polyclonal islet-specific Teff cells were harvested at day 7 (d7 islet Teff), and the remaining fraction of cells were expanded with IL-2 and harvested at day 14 (d14 islet Teff). Both d7 islet Teff and d14 islet Teff were used as Teff in the suppression assay (FIG. 154, panel A). The Teff were labeled with cell trace violet (CTV).

Monocyte-derived DC were generated by isolation of CD14+ cells from PBMC using CD14 microbeads (Miltenyi), then cultured in media supplemented with GM-CSF and IL-4 at 800 U/ml and 1000 U/ml, respectively, for 7 days to differentiate into mDC. These were later used as the APC in an antigen-specific suppression assay (FIG. 154, panel A).

The edTreg were generated from by CD4+CD25− T cells isolated from PMBC. The edTreg were labeled with EF670. The islet-specific edTregs included an IGRP-specific T1D2-TCR, or an GAD65-specific 4.13-TCR, which were specific for the IGRP₃₀₅₋₃₂₄ and GAD65₅₅₃₋₅₇₃, polypeptides, respectively. As indicated above, the IGRP₃₀₅₋₃₂₄ and GAD65₅₅₃₋₅₇₃, polypeptides were each present in the peptide pool used to generate the polyclonal islet-specific Teff cells.

In the suppression assay, the d7 islet Teff or d14 islet Teff were cultured with either no Treg, edTreg with endogenous polyclonal TCR, T1D2 mock, T1D2 edTreg, 4.13 mock, or 4.13 edTreg in the presence of mDC and a pool of 9 islet-specific peptides. Co-culture in the presence of mDC and DMSO was included as a negative control and showed no significant proliferation of Teff (data not shown). Results of this dye-dilution based assay demonstrated islet-peptide specific proliferation (FIG. 154C). Under antigen-stimulation conditions, polyclonal islet Teff proliferated specifically in the presence of mDC and the nine islet peptides after 4 days of incubation. Notably, islet peptide-specific proliferation was suppressed by both T1D2 edTregs and 4.13 edTregs. The edTregs with endogenous polyclonal TCR or mock islet-specific T cells regardless of their TCR did not show significant suppression of polyclonal islet enriched Teff. These results indicated that edTreg expressing islet TCR suppressed autoreactive islet-specific T cells with a variety of specificities in vivo from individuals with T1D.

### Example 18—Effects of TCR Avidity on edTreg Suppression Activity

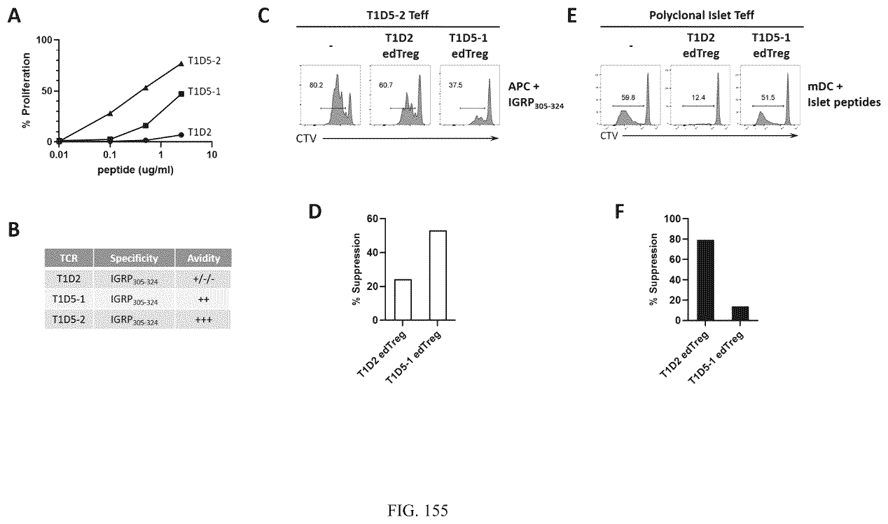

Effects of TCR avidity on suppressive activity of edTreg were determined. edTreg were generated with different TCRs which targeted the same antigenic peptide with different avidities. The TCRs were specific for an IGRP₃₀₅₋₃₂₄ peptide and included: T1D2, T1D5-1, and T1D5-2 (FIG. 155, panels A and B). edTreg were labeled with CTV and co-cultured with APC and serial dilutions of the IGRP₃₀₅₋₃₂₄ polypeptide. Four days after the co-culture, cells were stained and analyzed for proliferation. Co-culture in the presence of mDC and DMSO was included as a negative control and showed no significant proliferation of Teff. edTregs cells containing the T1D5-2 TCR demonstrated superior suppression of Teff proliferation compared to edTreg containing either T1D5-1 TCR or T1D2 TCR. This suggested that the edTreg with a higher avidity TCR had a greater suppression activity (FIG. 155, panels C and D). However, in a suppression assay, which included polyclonal islet-specific Teff, mDC and a pool of islet peptides, an edTreg containing a T1D2 TCR, exerted superior suppression activity compared to an edTreg containing a T1D5-1 TCR which had a higher avidity than the T1D2 TCR (FIG. 155, panels E and F).

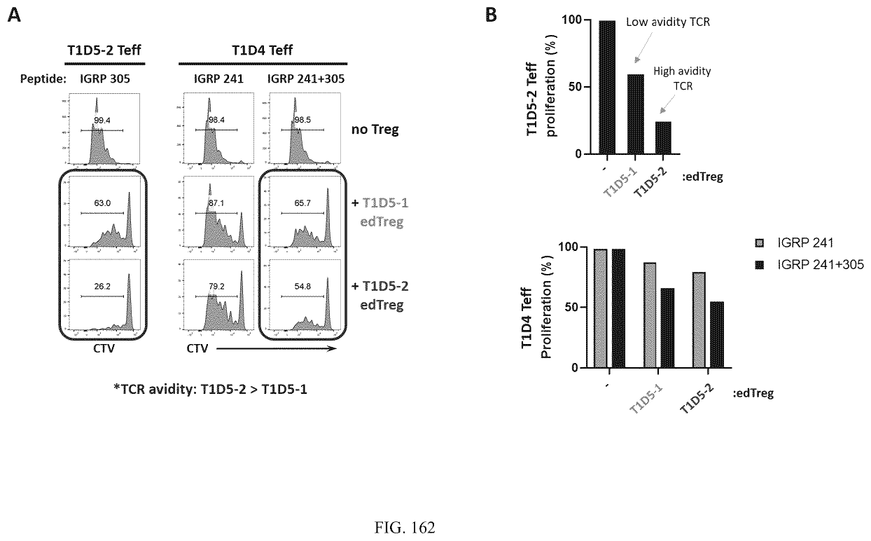

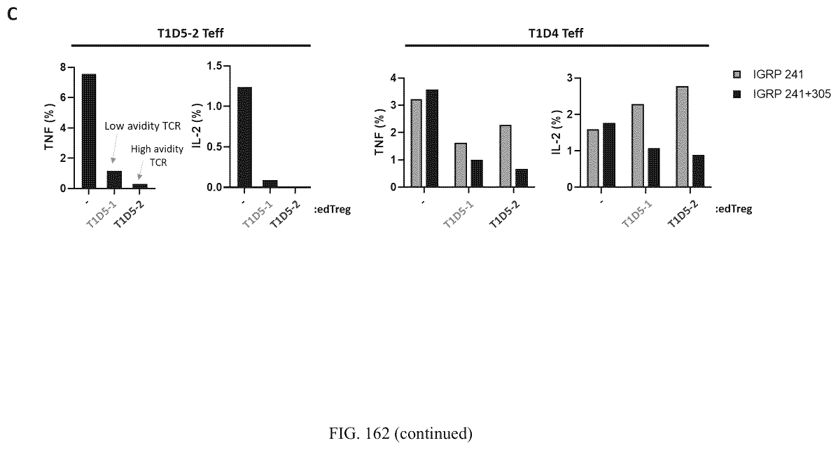

In a parallel experiment to demonstrate the superior suppressive capacity of edTreg with higher TCR activity, CTV-labeled T1D5-2 Teff or T1D4 Teff were co-cultured with or without EF670-labeled T1D5-1 edTreg or T1D5-2 edTreg in the presence of APC and IGRP 305, IGRP 241, or IGRP 241+305 peptides. Three days after the co-culture, cells were treated with BFA for four hours, stained, and analyzed (FIG. 162, panel A). T1D5-2 had a higher TCR activity than TID5-1; consistently, TID5-2 had significantly higher suppression of polyclonal islet Teff proliferation (FIG. 162, panel B). This also corresponded with cytokine production; the higher TCR activity of TID5-2 correlated inversely with the production of TNF and IL-2 (FIG. 162, panel C). Collectively, this data showed that TCR activity in edTreg significantly influenced suppression of both polyclonal islet Teff proliferation and cytokine production.

As disclosed herein, islet-specific edTreg suppressed polyclonal islet-specific T cells derived from PBMC. CD4+ T cells transduced with lentiviral T1D2, T1D4, T1D5-1, T1D5-2, 4.13, GAD₁₁₃, or PPI₇₆ TCR were labeled with CTV and co-cultured with APC and their cognate peptide with serial dilutions. Four days after the co-culture, cells were stained and analyzed for proliferation (FIG. 156A, panel A). Similar to the above case, T1D2, T1D4, T1D5-1, T1D5-2, 4.13, GAD₁₁₃, and PPI₇₆ TCR were all specific for IGRP₃₀₅₋₃₂₄ peptide, but showed different avidity (FIG. 156A, panel B). Additional results are shown in FIG. 156B. Polyclonal islet Teff were cultured with no Treg, T1D2 edTreg, or 4.13 edTreg in the presence of mDC and a pool of 9 islet-specific peptides (FIG. 156A, panel C). There was superior suppression of Teff proliferation by 4.13 edTreg than T1D2 edTreg, although not as superior as T1D5-1 edTreg shown in FIG. 155, panel C. This suggested that the edTreg with the highest avidity TCR was most effective. When polyclonal islet Teff were cultured with no Treg, T1D2 edTreg, GAD113 edTreg or PPI edTreg in the presence of mDC and a pool of 9 islet-specific peptides, the lower avidity TCR exerted superior suppression on Teff proliferation in comparison to the others. Consistent with the previous results, T1D2 edTreg, the edTreg with the lowest avidity TCR, showed the highest suppression of polyclonal islet Teff proliferation.

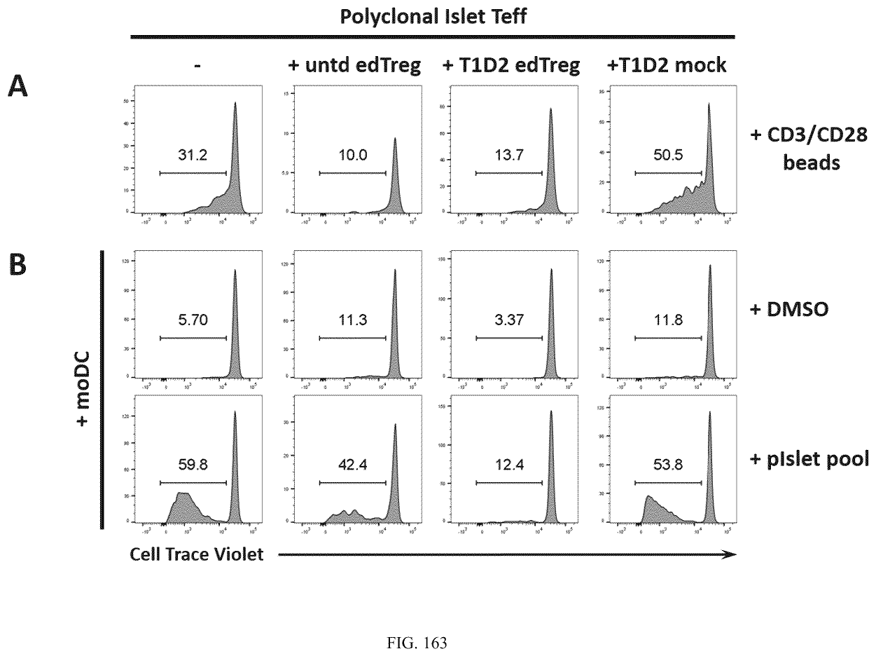

The superior suppressive function of edTreg on polyclonal islet Teff was further demonstrated through examining additional TCRs. CD4+CD25− T cells were isolated from PBMC and co-cultured with 9 islet-specific peptides (5 GAD65-specific peptides, 3 IGRP peptides, 1 PPI-specific peptide) and irradiated CD4−CD25+ cells for 7 days for generation of islet-specific CD4+ T cells. Islet-specific T cells and edTreg or mock cells were labeled with CTV and EF670, respectively. Islet-specific T cells were incubated with no Treg, untd edTreg, T1D2 edTreg, or T1D2 mock cells in the presence of CD3/CD28 activator beads. Four days after the co-culture, cells were stained and analyzed (FIG. 163, panel A). The islet-specific edTreg significantly suppressed the polyclonal islet T cells. Similarly, the same islet-specific T cells were co-cultured for 4 days with no Treg, untd edTreg, T1D2 edTreg, or T1D2 mock cells in the presence of monocyte-derived DC and a pool of 9 islet-specific peptides or DMSO (FIG. 163, panel B). Following the same trend as observed above, in the presence of mDC and a pool of 9 islet-specific peptides, the lower avidity TCR exerted superior suppression.

### Example 19—Modified TCR Activity and Islet-Specific edTreg Enhance Suppressive Activity

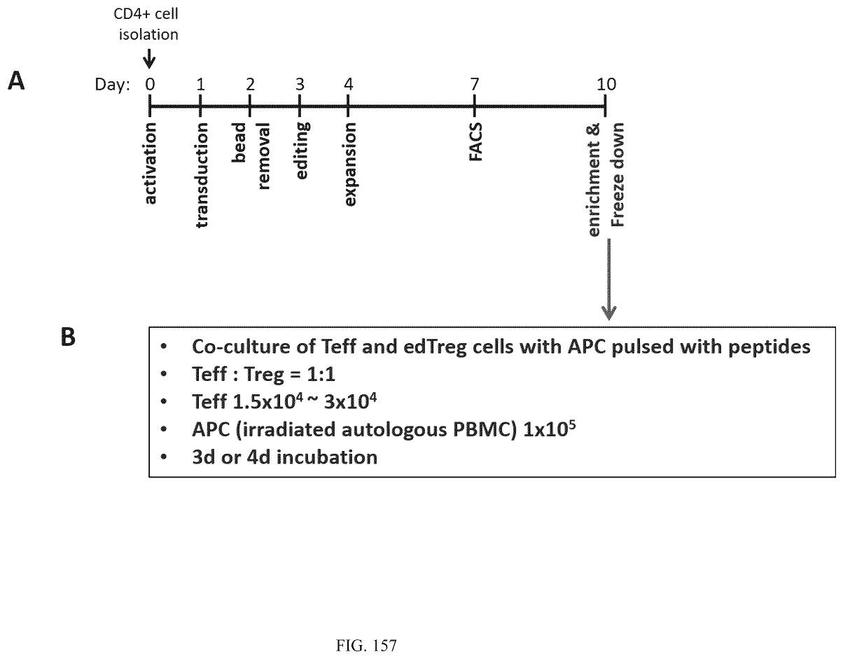

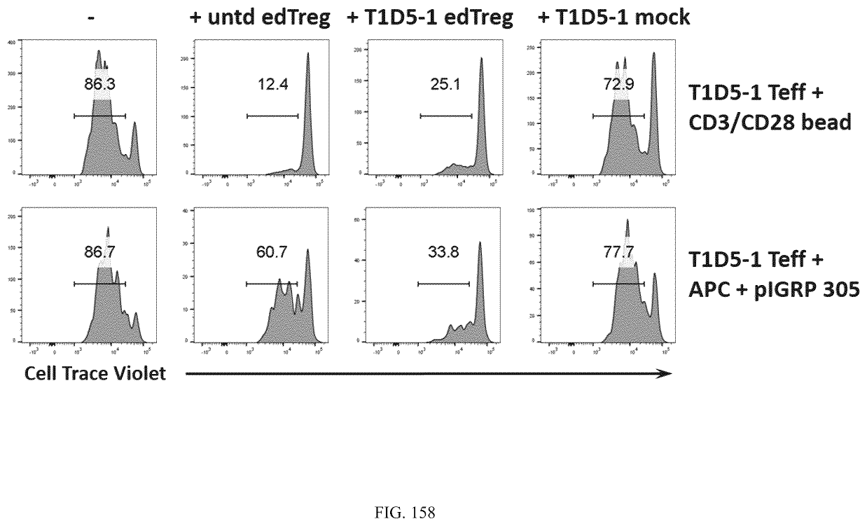

Certain edTreg cells expressing islet-TCRs were generated. edTreg cells with islet-TCRs (no LV TCR, T1D4, or T1D5-1 TCR) were enriched by LNGFR expression using MACS LNGFR beads. LNGFR+ cells were aliquoted and frozen down for further experiments. A timeline of the methodology for generating Treg cells and assessing antigen-specific suppression is depicted in FIG. 157, panels A and B. Briefly, CD4+ T cells were transduced with islet-TCRs (T1D4 or T1D5-1 TCR), which were then used as Teff cells. Teff cells and Treg cells were labeled with different reagents—for example CTV or EF670- and co-cultured with or without edTreg cells at a 1:1 ratio in the presence of APC (autologous irradiated PBMC) and various peptides. Cells were stained and analyzed by flow after 3-4 days following incubation for measuring cytokine generation and proliferation of Teff cells. Teff cell proliferation in response to CD3/CD28 bead activation and antigen-specific proliferation were analyzed (FIG. 158). CD4+ T cells transduced with T1D5-1-TCR (T1D5-1 Teff) were labeled with CTV and co-cultured with or without EF670-labeled untransduced edTreg (untd edTreg), edTreg expressing T1D5-1 TCR (T1D5-1 edTreg), or T1D5-1 mock cells in the presence of CD3/CD28 activator beads. Same T1D5-1 Teff with or without untd edTreg, T1D5-1 edTreg, or T1D5-1 mock cells were co-cultured with APC and IGRP 305 peptide. Three days after the co-culture, cells were stained and analyzed for Teff proliferation as dilution of CTV. Both edTreg and T1D5-1 edTreg cells demonstrated significant suppressive activity of Teff proliferation compared with mock cells.

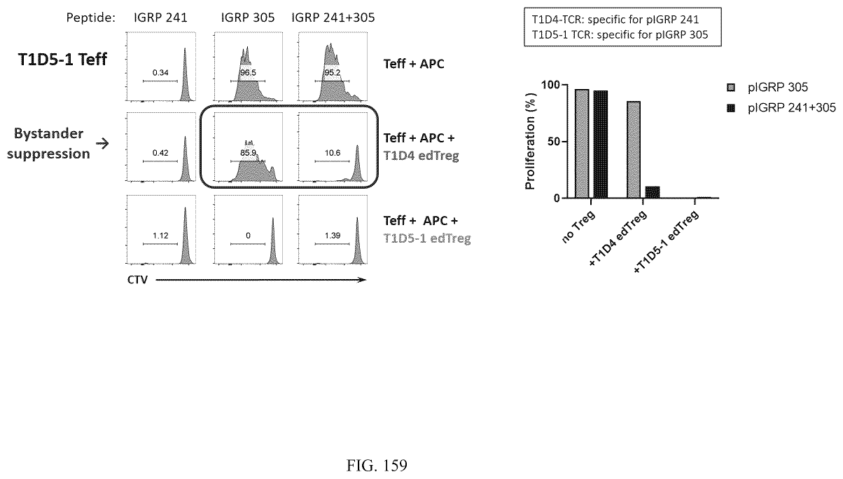

Antigen-specific and bystander suppression on Teff by edTreg was assessed (FIG. 159). Teff and Treg cells were labeled with CTV and EF670, respectively. T1D5-1 Teff were co-cultured with or without T1D4 edTreg or T1D5-1 edTreg in the presence of APC and various peptides (IGRP 241, IGRP 305, or IGRP₂₄₁+IGRP 305). Four days after the co-culture, cells were stained and analyzed for Teff proliferation as dilution of CTV. T1D4 TCR was specific for pIGRP 241, while T1D5-1 TCR was specific for pIGRP 305. T1D4 TCR did not show significant suppression when in contact with IGRP 305 but induced significant suppression on Teff with IGRP 241 and IGRP₂₄₁+IGRP₃₀₅. Conversely, T1D5-1 TCR demonstrated significant suppression on Teff in all three peptide exposures. This analysis demonstrated significant bystander suppression by T1D4 edTreg.

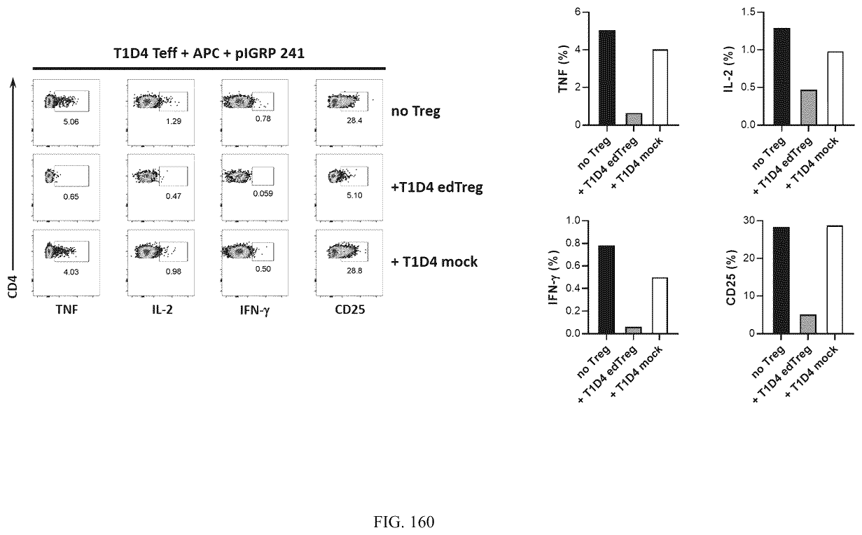

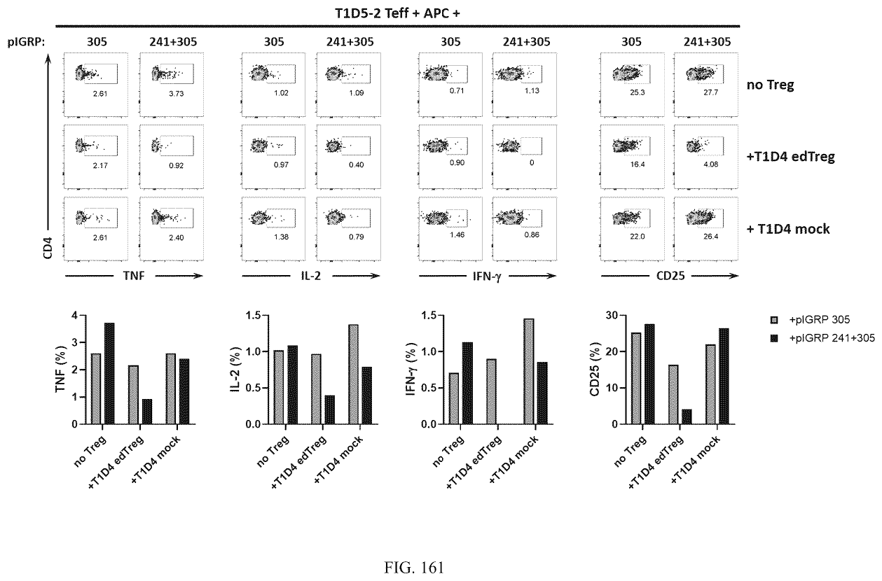

Cytokine production in both Teff and bystander Teff by edTreg was examined (FIG. 160 and FIG. 161). Teff and Treg cells were labeled with CTV and EF670, respectively. T1D4 or T1D5-2 Teff cells were co-cultured with or without T1D4 edTreg or mock cells in the presence of APC and IGRP 241 peptide, or IGRP 305 and IGRP 241/305 peptides. Three days after the co-culture, cells were treated with BFA for 4 hours, stained, and analyzed for cytokine generation from Teff cells. Similar to the above results, TID4 Teff and TID4 edTreg cells in the presence of IGRP 241 significantly suppressed cytokine production compared to mock cells. Conversely, T1D5-2 Teff cells with TID4 edTreg cells had much lower cytokine production in the presence of both IGRP 241+305 peptides compared with IGRP 305 alone. Collectively, this data indicated that Teff by edTreg causes significant suppression of cytokine production.

### Example 20—Generation of Engineered EngTregs from Umbilical Cord Blood-Derived CD4+ T Cells

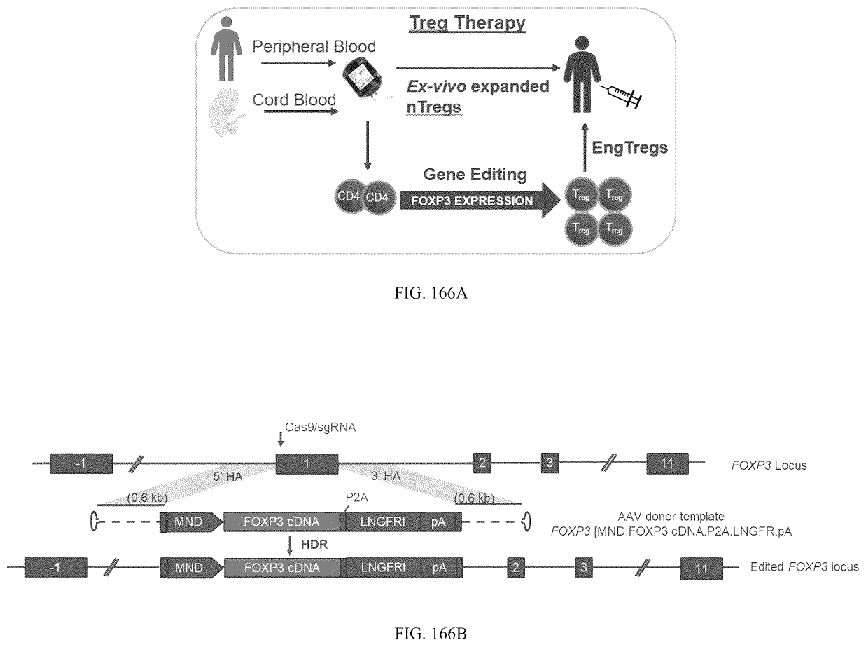

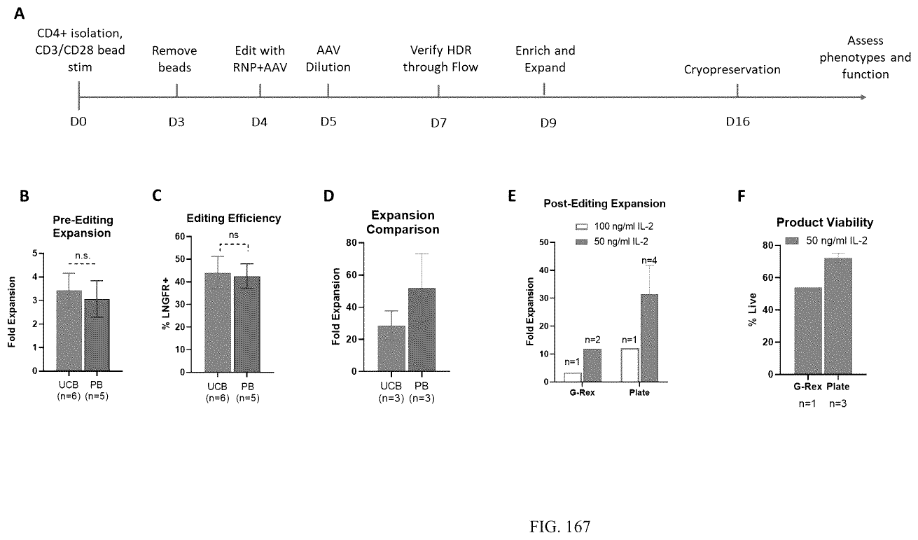

Umbilical cord blood (UCB)-derived EngTregs were produced by isolating CD4+ cells from umbilical cord blood, and introducing an expression cassette comprising FOXP3 cDNA under the control of an MND promoter into the FOXP3 locus at the first coding exon (FIGS. 166A-166B). Cells expressing CD4 were isolated, then CD3 and CD28 on the cells were stimulated using CD3/CD28 Dynabeads. Following removal of beads, cells were edited by the introduction of a ribonucleoprotein complex containing a Cas9 protein and sgRNA, and infection with an AAV vector containing a donor template for integration of an MND-FOXP3 cDNA expression cassette into the FOXP3 locus (FIG. 167). After editing was confirmed by flow cytometry, edited cells were enriched and expanded by stimulation with IL-2 to induce proliferation. UCB-derived EngTregs exhibited similar editing and expansion efficiencies to EngTregs derived from circulating CD4+ cells from peripheral blood (PB) (FIG. 167). Expansion and cell survival was improved by culture in tissue culture plates, compared to a G-rex device (FIG. 167).

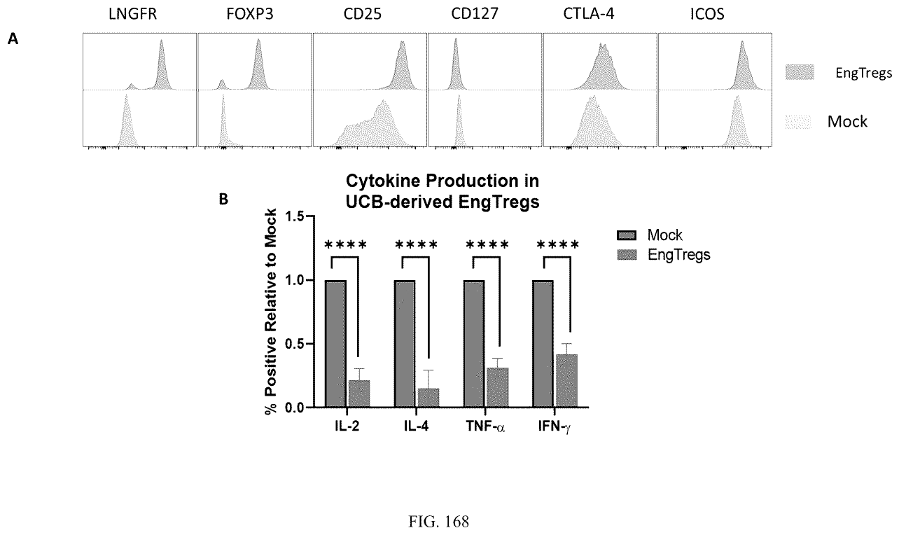

The immunophenotype of UCB-derived EngTregs was evaluated by flow cytometry analysis, following staining of cells for surface markers and intracellular staining for the production of pro-inflammatory cytokines. Compared to mock-edited cells, UCB-derived EngTregs expressed more FOXP3 and CD25, indicating conversation to a FOXP3+CD25+ Treg phenotype (FIG. 168). UCB-derived EngTregs also produced markedly lower levels of IL-2, IL-4, TNF-α, and IFN-γ following PMA/ionomycin stimulation than mock-edited cells did, indicating a downregulation of pro-inflammatory activity.

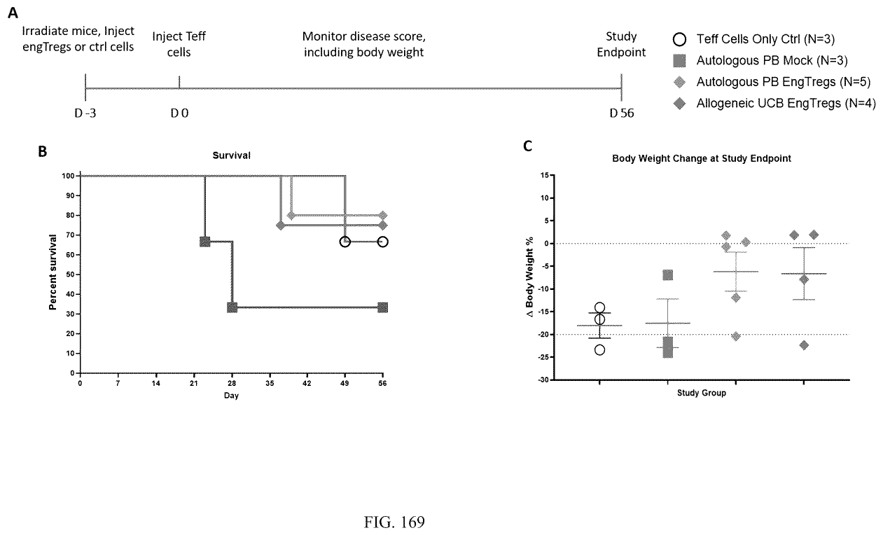

The capacity of UCB-derived EngTregs to protect against autoimmune pathology in vivo was evaluated in a mouse model of graft-vs-host disease (GvHD). NSG mice were irradiated, then engrafted with autologous mock-edited PB CD4+ cells, autologous PB-derived EngTregs, or allogeneic UCB-derived EngTregs. 3 days later, all mice were injected with allogeneic effector (Teff) cells, which cause pathology similar to that of GvHD in this mouse model. Mice were monitored for signs of disease, weight loss, and survival over 56 days. UCB-derived EngTregs protected mice from death and weight loss with similar effectiveness to PB-derived EngTregs (FIG. 169). Thus, UCB-derived EngTregs are effective at alleviating the symptoms of autoimmune and inflammatory conditions in vivo.

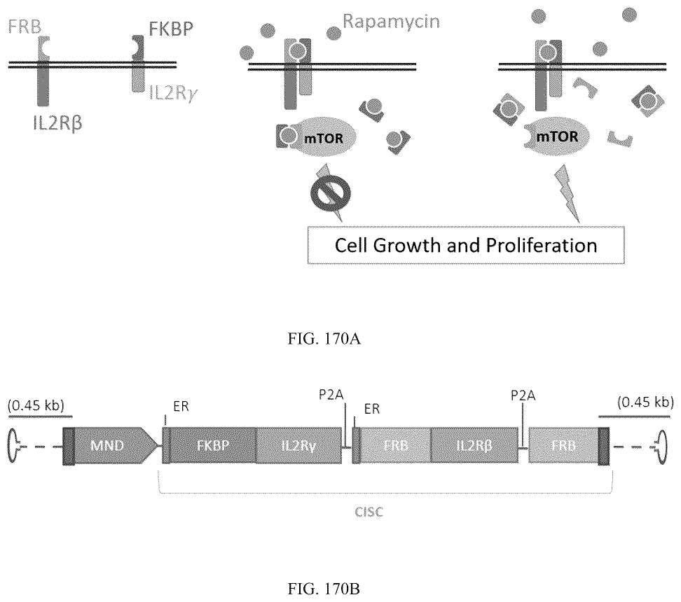

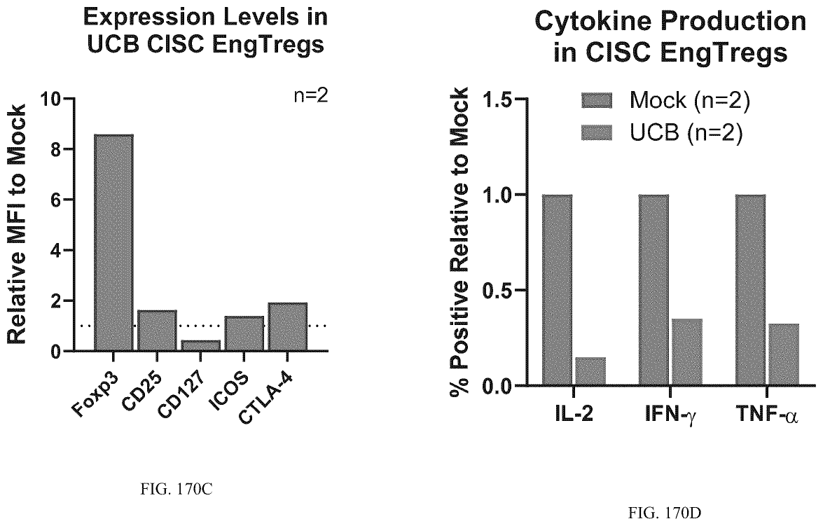

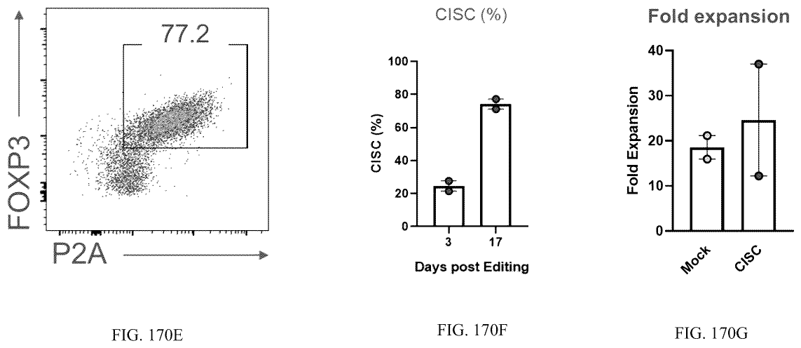

UCB-derived EngTregs were also produced by an alternative gene editing approach, in which an expression cassette driven by an MND promoter and encoding an FKBP-IL2Rg polypeptide, FRB-IL2Rb polypeptide, and FRB polypeptide was inserted into the FOXP3 locus at the first coding exon. Each polypeptide is separated by a P2A self-cleavage motif, and is in-frame with the open reading frame encoding FOXP3, such that the MND promoter drives transcription and subsequent translation of one polypeptide containing 1) FKBP-IL2Rg polypeptide, 2) FRB-IL2Rb polypeptide, 3) FRB polypeptide, and 4) FOXP3 (FIG. 170B). Following translation, each of the four components is cleaved by the P2A self-cleavage domain, such that the edited cell expressed FOXP3, both components of a CISC that simulate IL-2 signal transduction in the presence of rapamycin or a rapalog, and an intracellular FRB protein to prevent excess rapamycin or rapalog from stimulating intracellular mTOR (FIG. 170A). Flow cytometry confirmed that UCB-derived EngTregs expressed both FOXP3 and CISC components, based on the presence of the P2A self-cleavage motifs (FIG. 170E). EngTregs expressing this CISC and intracellular FRB protein exhibited upregulation in FOXP3 expression, and lower levels of pro-inflammatory cytokine production upon PMA/ionomycin stimulation, relative to mock-edited UCB-derived CD4+ cells (FIGS. 170C-170D). CISC-expressing UCB-derived EngTregs could be enriched by exposure to rapamycin, indicating successful expression of both CISC components that dimerize in the presence of rapamycin (FIGS. 170F-170G).

### Example 21—Generation of Engineered Tregs (EngTregs) from Umbilical Cord Blood Derived CD4+ T Cells Via HDR-Mediated FOXP3 Gene Editing

An additional in vivo study was performed to compare allogeneic UCB EngTregs vs autologous PB Mock edited cells or autologous PB EngTregs in xenogeneic GvHD mouse model. This study was performed using UCB EngTregs from two different donors. The in vivo study showed that the EngTregs derived from allogeneic UCB significantly delayed GvHD onset in NSG mice, increasing the overall survival rate of mice and protecting animals from weight loss associated with GvHD. This data clearly showed that UCB allogeneic EngTregs exerted robust suppressive activity similar to autologous EngTreg derived from peripheral blood sources. This data provided further evidence that efficacy was reproducible and not donor dependent.

### In Vitro and In Vivo Characterization of UCB-Derived EngTreg Product—Repeat Experiment

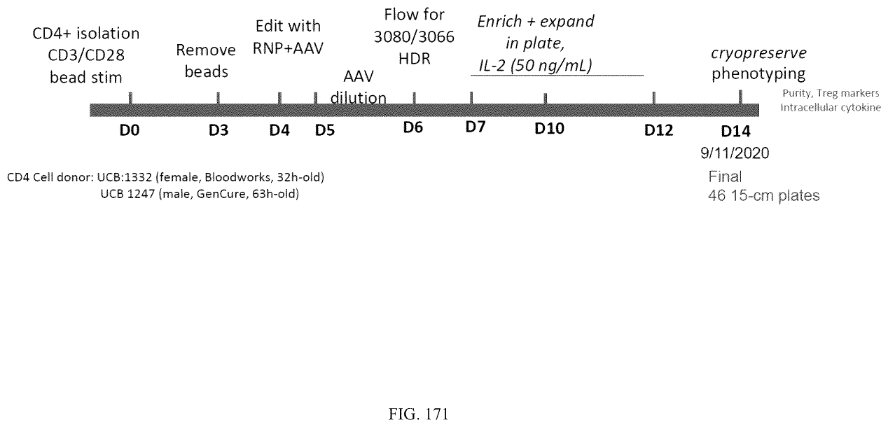

In this study, a procedure outlined herein was used to edit, enrich, and expand UCB CD4 T cells for generating two UCB EngTreg products from different UCB donors. The timeline and procedure for editing and expansion is outlined in FIG. 171. Briefly, FIG. 171 includes: CD4 isolation: CD4+ T from UCB MNCs using Easysep CD4 negative isolation #19052; CD4 activation: 0.5 million cells/ml with 3:1 bead-to-cell ratio using Dynabeads™ Human T-Expander CD3/CD28 (Thermo Fisher (11141D); Culture media: RPMI1640 with 20% FBS, HEPES, GLUTAMAX, β-Mercaptoethanol, IL-2 (50 ng/ml); Nuclease and guide: Aldervon Spyfi Cas9 (Research grade)+Biospring T9 guide (50 uM stock) @1:2.5 (20 pmol: 50 pmol); RNP delivery: Lonza, EO-115; AAV: AAV6 #3066 (MND-FOXP3cDNA-LNGFR); Enrichment: CD271 Microbead (Miltenyi #130-099-023), LS column (Miltenyi #130-042-401); with minor modifications of manufacture's protocol; Expansion: expand mock and enriched EngTregs with 3:1 T-expander beads in plates; Expansion media: RPMI1640 with 20% FBS, HEPES, GLUTAMAX, β-Mercaptoethanol, IL-2 (50 ng/mL), rapamycin (100 nM, one-time treatment at plating); split/expand on 3rd and 5th day of expansion; no additional beads or rapamycin added during the 7-day expansion; Cryopreservation: freeze expanded mock and enriched EngTregs in Cryostor CS10 after bead removal.

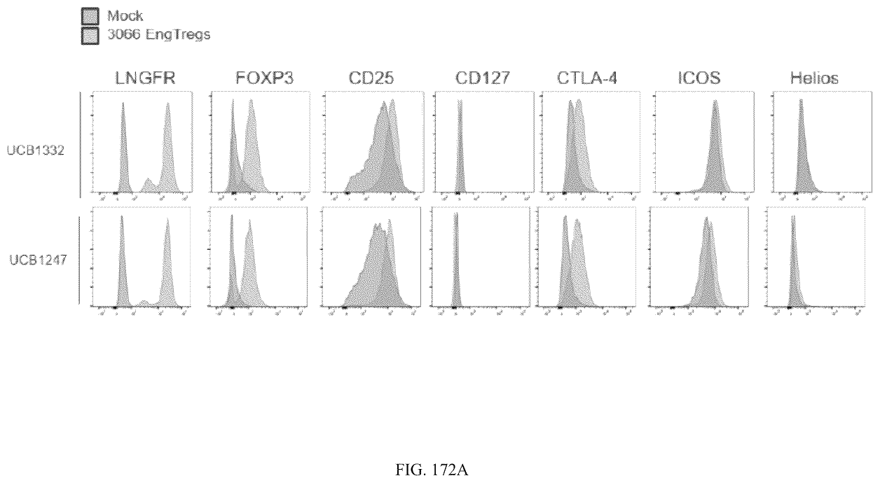

For this study, UCB and PB cells were edited with AAV6 #3066 (MND-FOXP3cDNA-LNGFR). It was found that: 1) pre-editing fold expansion, 2) editing efficiency, 3) purity and 4) post enrichment expansion was nearly identical with both UCB donors and similar to previous data. Likewise, the UCB-derived EngTregs from both donors displayed Treg immunophenotype (high levels of FOXP3 and LNGFR, as well as CD25, CTLA-4 and ICOS, and concomitant reduction in inflammatory cytokine production (IL-2, IL-4, TNF-alpha, or INF-gamma) as assessed by the response to PMA/ionomycin stimulation (FIG. 172A, FIG. 172B), consistent with a Treg-like phenotype and EngTregs derived from PB.

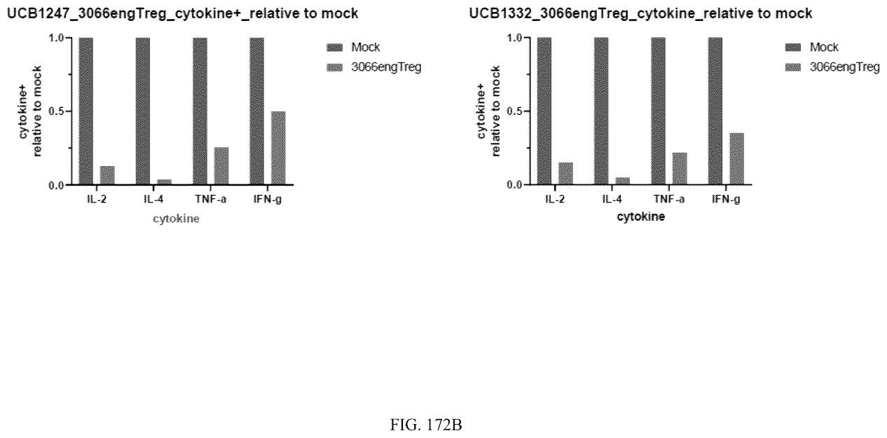

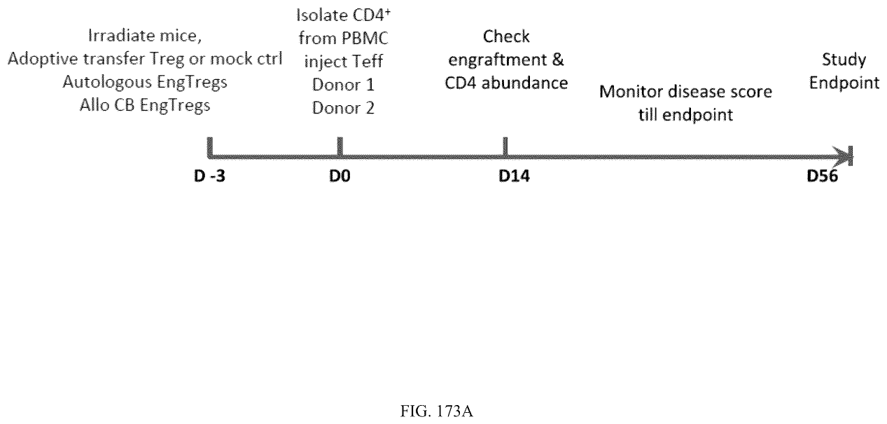

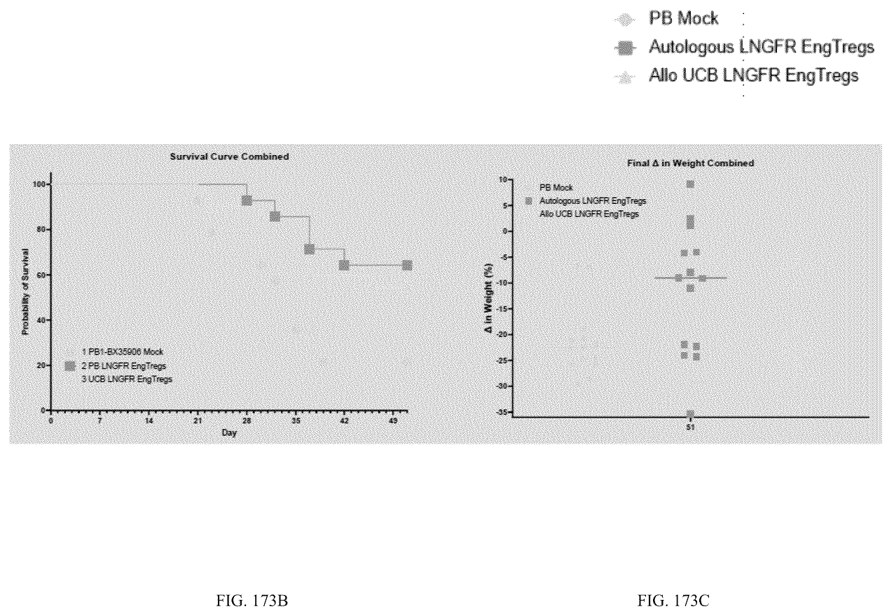

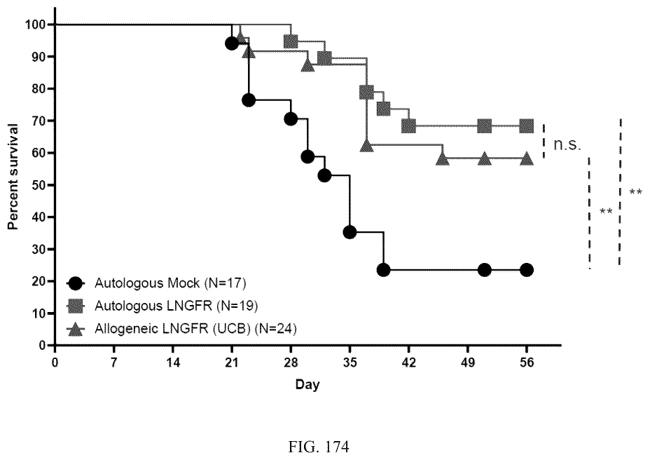

In vivo functional activity of the UCB and PB EngTreg products derived from production protocols provided herein from two individual donors was evaluated. Autologous PB Mock edited cells, autologous PB EngTregs and allogeneic UCB EngTregs were compared in xenogeneic GvHD mouse model. The study design is shown in FIG. 173A and the results are shown in FIG. 173B and FIG. 173C. The in vivo study showed that the EngTregs derived from PB and UCB delay GvHD onset in NSG mice, increased the overall survival rate of mice and protected animals from weight loss associated with GvHD, consistent with the data described herein. The survival curve of NSG mice in combined xenoGvHD studies is shown in FIG. 174, and represents the combined results of 2 CB EngTreg products against 3 different allogenic PB-derived CD4 Teffs populations. P values were calculated using Log-rank (Mantel-Cox) test which demonstrated a significant difference between autologous mock edited PB compared to autologous LNGFR PB and allogeneic LNGFR UCB, but no significance between the LNGFR PB compared to LNGFR UCB.

The results of the in vivo xenogeneic GvHD study described here using 2 independent UCB donors, was consistent with a previous study and definitively showed that UCB EngTregs exhibited significant protective effects in vivo in a highly relevant model of autoimmunity. These products increased the overall survival rate of mice and protect these animals from weight loss associated with GvHD driven by activated allogeneic CD4 effector T cells. Importantly, these studies demonstrated that UCB EngTreg performed equivalently to autologous CD4 EngTreg derived from the peripheral blood T cells of the donor driving GvHD, supporting their clinical application in allogeneic settings.

### Example 22—Generation of FOXP3-Expression Engineered Regulatory T Cells in CD4+ and CD8+ T Cell Populations Using a Gene Editing Approach in CD3+ T Cells

Having established that lentiviral delivery of MND-driven FOXP3 into CD3+ T cells can convert CD8+ T cells to CD8+ EngTregs, CD4+ and CD8+ EngTregs generated through a gene editing approach were compared. Compared with LV delivery, the HDR-based gene editing approach was predicted to lead to a more stable/robust CD8+ EngTreg population due to epigenetic silencing and/or integration dependent variegation of expression of the integrated LV cassette.

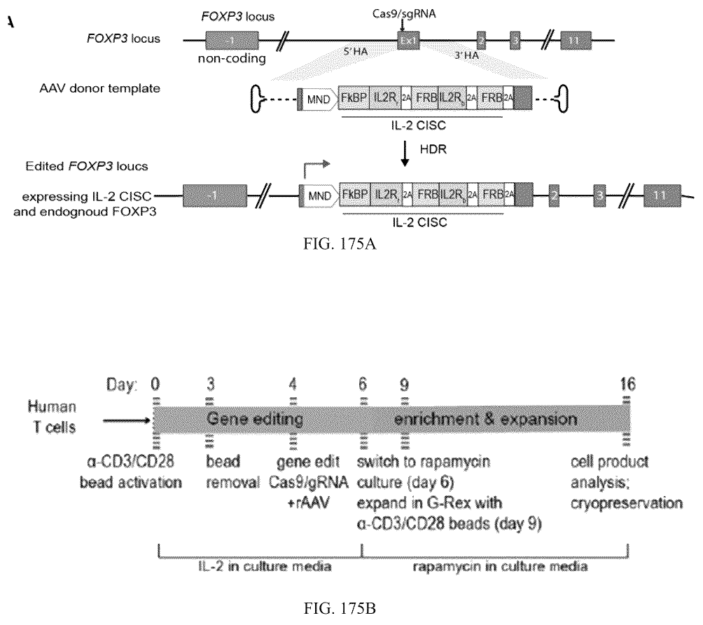

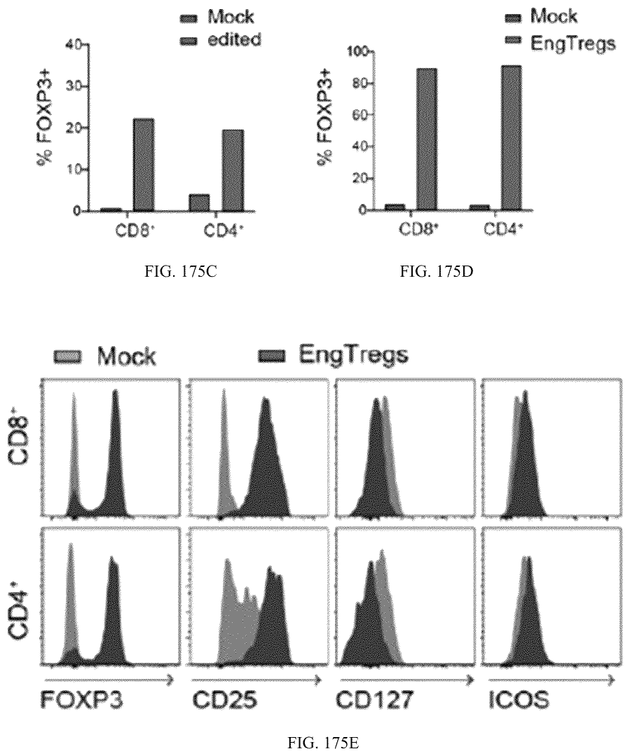

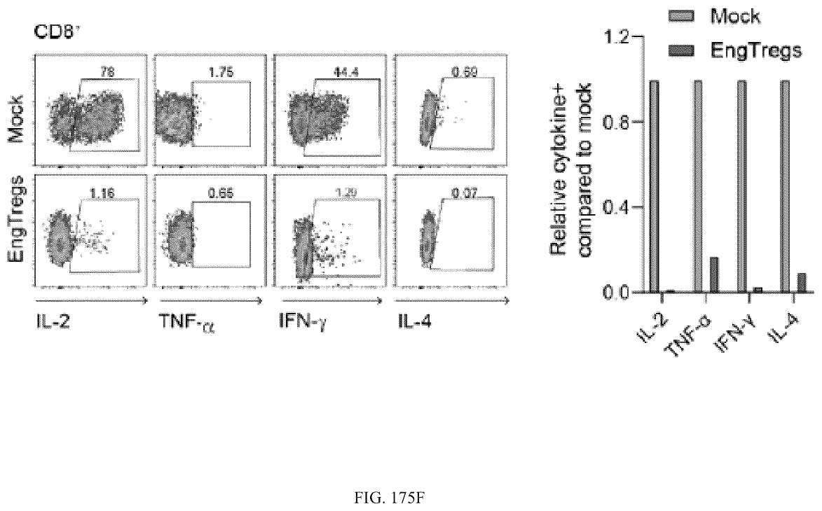

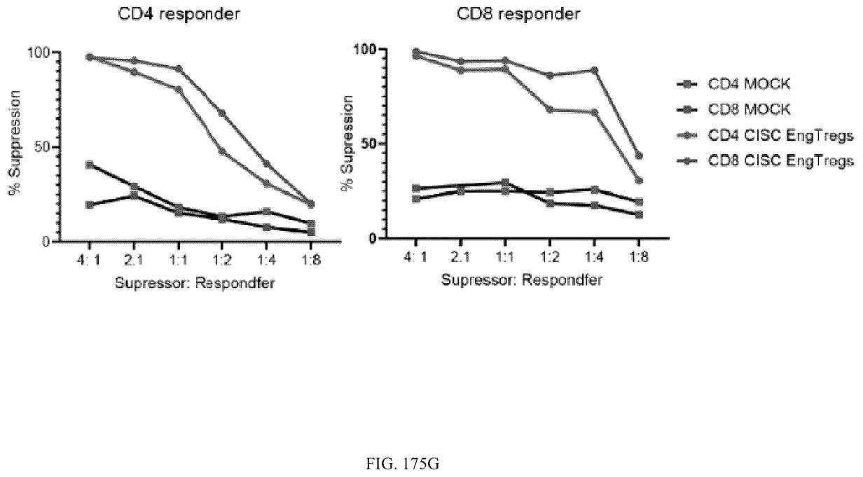

Generation of EngTregs using a gene editing approach with CISC containing and non-CISC containing constructs in CD4+ T cells has been demonstrated. In the experiments described herein, the same approach to edit CD3+ T cells was performed, and the Treg properties in both CD4+ and CD8+ T cell subsets of the edited product were compared. FIG. 175A depicts AAV construct MND.IL-2.CISC (#3195) which was used to introduce the CISC cassette into the FOXP3 locus in CD3+ T cells, and depicts the timeline for the study (FIG. 175B). The CD8+ and CD4+ population in mock edited and #3195 edited CD3+ T cells, 2 days post editing, were similar. Specifically, for mock edited cells: 52.9% CD8+; 44.1% CD4+; for #3195 edited CD3+ T cells: 58.2% CD8+; 39.2% CD4+. Editing efficiency as measured by FOXP3 expression in each T cell subset was similar: 22.3% and 19.8% for CD8+ and CD4+ respectively. FIG. 175C. Importantly, a high level of purity of CD8+ EngTregs was reached with rapamycin enrichment and expansion (90.4% FOXP3+/CD25+ cells), similar to CD4+ (91.6% FOXP3+/CD25+) (FIG. 175D). CD4+ and CD8+ EngTregs cell populations had a reduction of inflammatory cytokines compared to mock edited cells (FIG. 175F) and demonstrated a Treg phenotype with high FOXP3, CD25, and ICOS, and low CD127 (FIG. 175E). In addition, in vitro suppression studies using CD4 and CD8 EngTregs demonstrated significant suppression of CD4 and CD8 responder cells, compared to CD4 and CD8 mock cells, with CD8 EngTregs having slightly more suppressive activity than CD4 EngTregs (FIG. 175G).

### Generation of CD8 EngTregs Using a Gene Editing Approach in CD8+ Purified T Cells

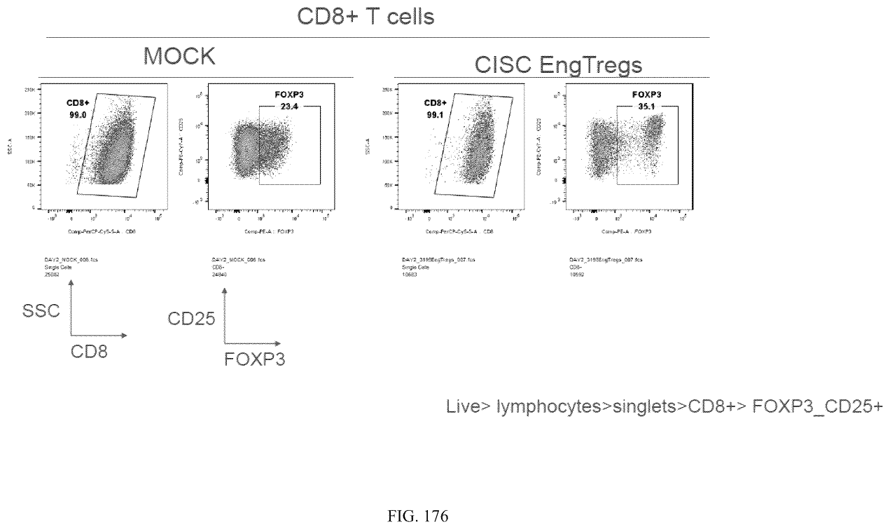

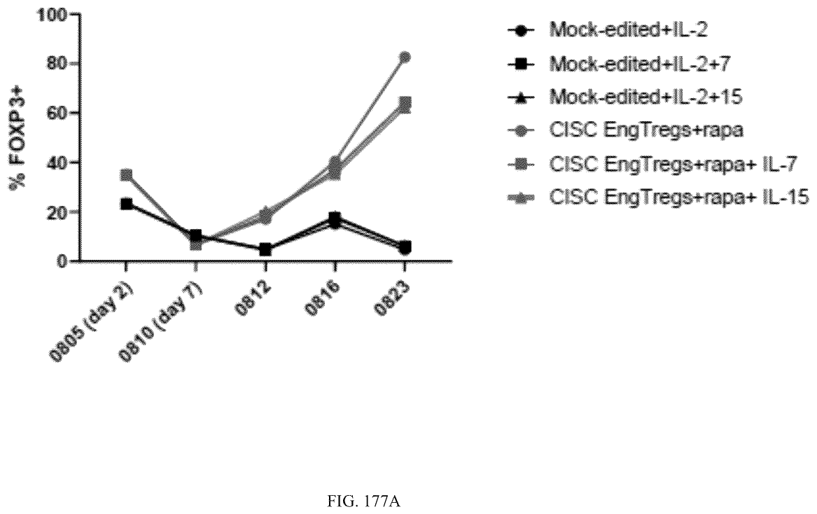

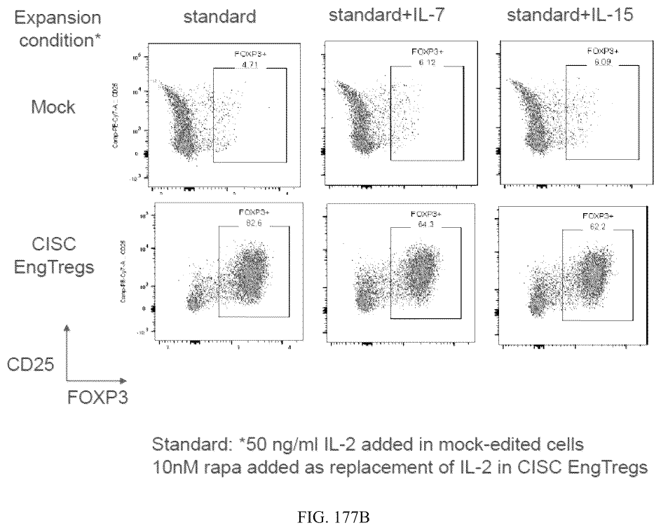

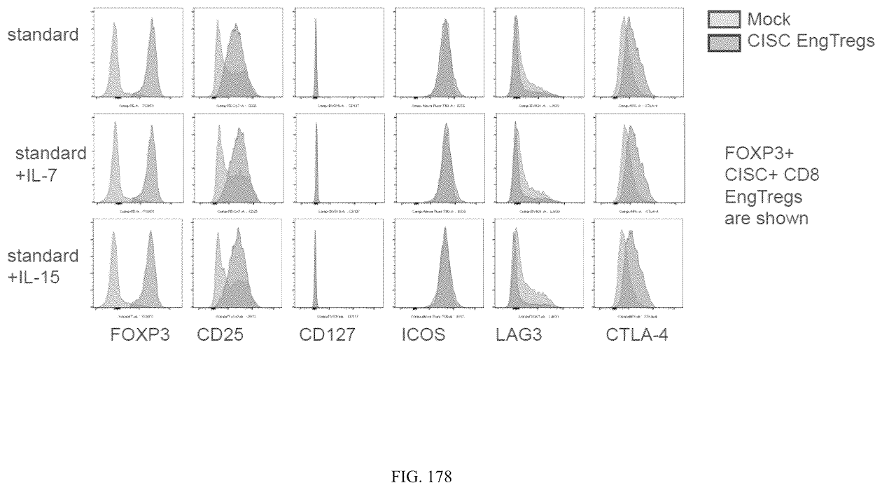

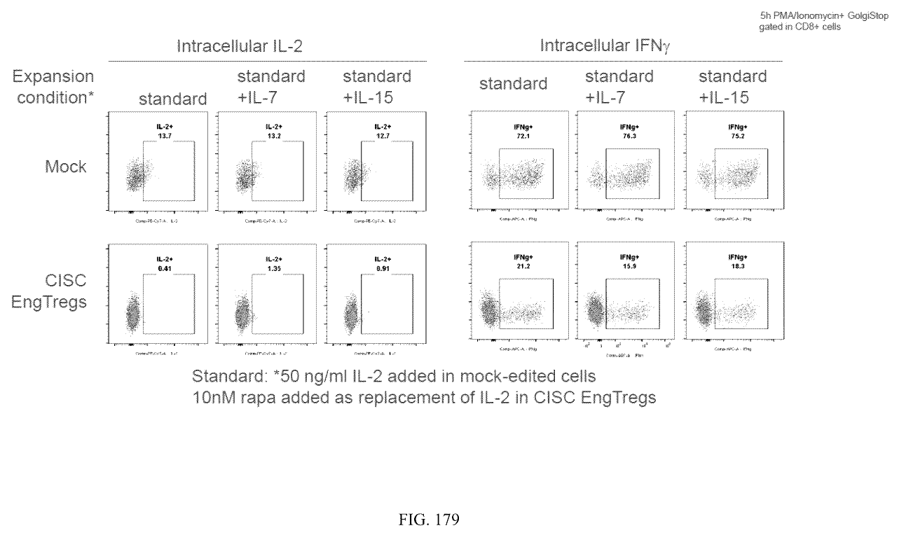

The results shown in FIGS. 175A-175F, demonstrated that the CD8+ T cell population of CD3+ FOXP3 edited cells, had a phenotype and suppressive activity similar CD4+ Treg. To determine if FOXP3-expressing engineered regulatory T cells from a purified CD8+ T cell population can be generated, the construct and editing/expansion procedure as depicted in FIG. 175A and FIG. 175B was used, on purified CD8+ T cells rather than CD3+ cells. FIG. 176 depicts the editing efficiency as measured by FOXP3 expression was 35.1% in purified CD8+ T cells edited with the MND.IL-2.CISC construct compared to 23.4% in mock edited cells. The minimal FOXP3 expression in mock edited cells was caused by transient expression of FOXP3 T conv upon cell activation. Note, that after several days in culture, FOXP3 transient expression went away. In contrast, CISC EngTregs reached 82.6% FOXP3+ purity following the addition of rapamycin for 20 days compared to 4.7% FOXP3+ cells in mock edited CD8+ T cells treated with IL-2 (FIG. 177A, FIG. 177B). In addition to rapamycin alone, rapamycin plus IL-7 or IL-15 was also tested as various studies have suggested that IL-15 may be beneficial in cultures of CD8 T cells including memory populations. Results demonstrated that IL-7 and IL-15 had no additional impact on the purity of CISC EngTregs. This suggested that IL-7 and IL-15 were not needed for CD8 EngTreg generation and based upon the enrichment curves, these cytokines may have had a negative impact on the expansion of CD8 EngTregs. Further analysis of the CD8+ EngTregs (derived from purified CD8+ T cells), demonstrated a similar phenotype (FIG. 178) and cytokine production FIG. 179) as seen with CD8+ EngTregs and CD4+ EngTregs (derived from CD3+ T cell editing, shown in FIG. 175). Mock edited cells and CISC EngTregs treated with IL-7 and IL-15 showed similar immune markers and cytokine profile as cells in the standard culture conditions indicating that these cytokines were not necessary for generation of CD8 EngTregs with Treg characteristics.

### Generation of CISC Expressing CD8 EngTregs Using a Dual Editing Approach

As described herein, a CISC containing CD4+ EngTreg product using a dual editing approach generated antigen-specific EngTregs. This approach used split-CISC constructs expressing the FKBP-IL2RG component and FRB-IL2Rb, and edited into the TRAC and FOXP3 locus thereby permitting the selection of only dual edited cells using the CISC system. Dual locus editing was used to generate EngTregs that express alternative antigen or other targeting moieties including CARs and/or other receptors or tissue homing entities. In the studies outlined below, using the A2-CAR split-CISC constructs shown, the ability to dual-edit CD3+ and CD8+ T cells to generate a A2CAR CISC-expressing dual-edited CD8+ EngTregs was demonstrated.

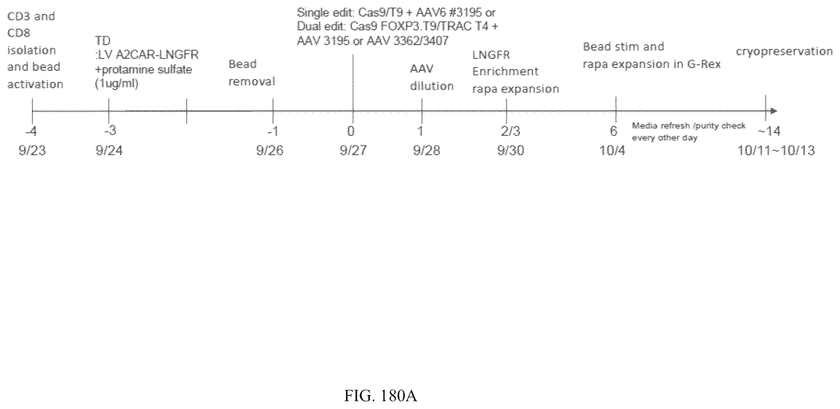

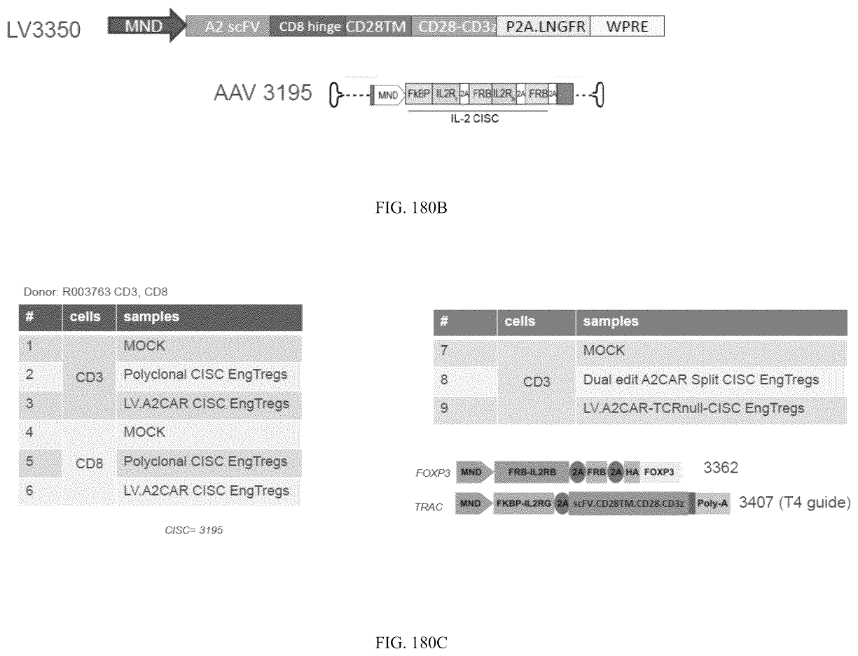

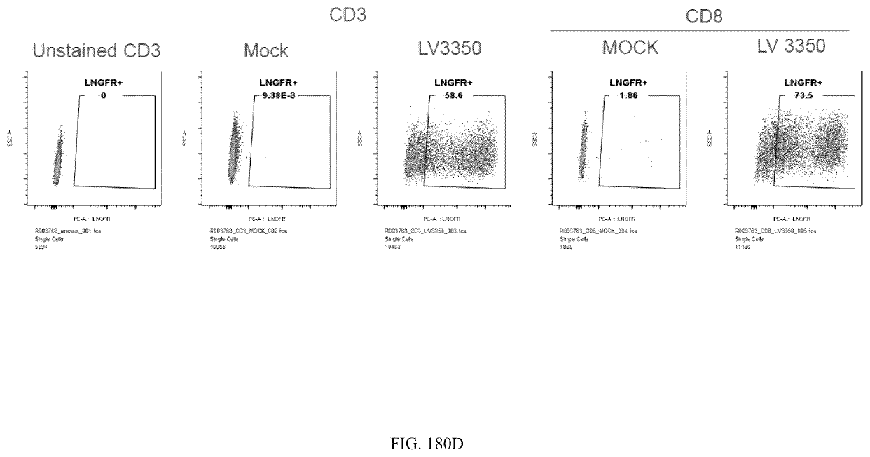

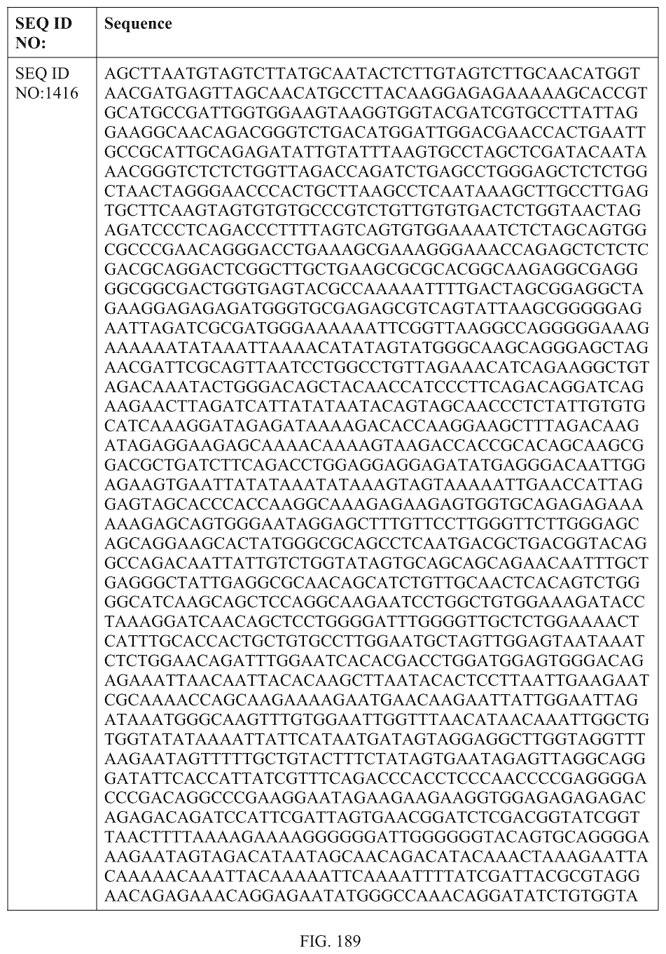

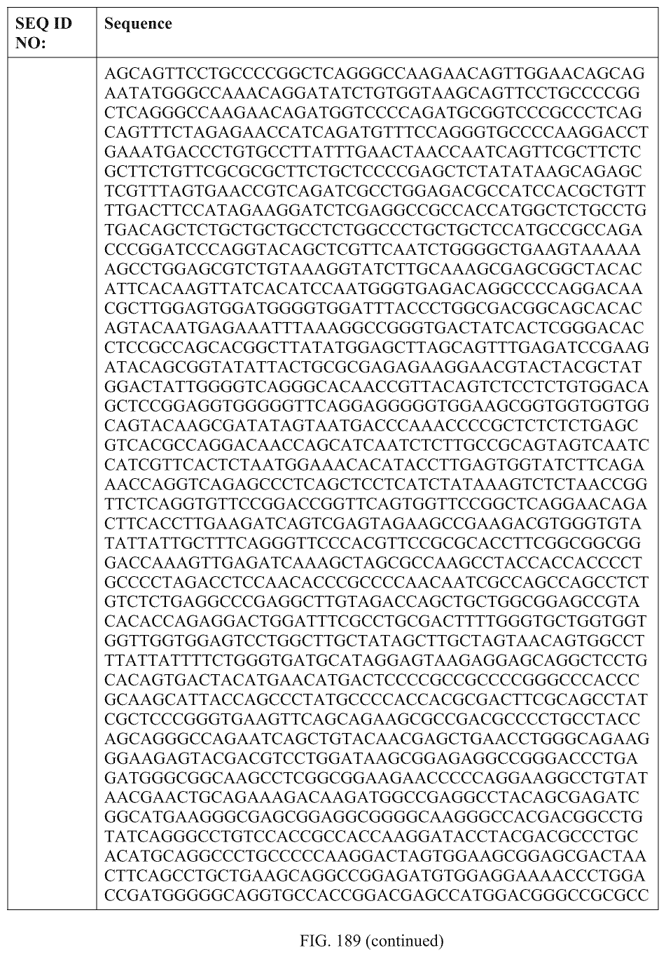

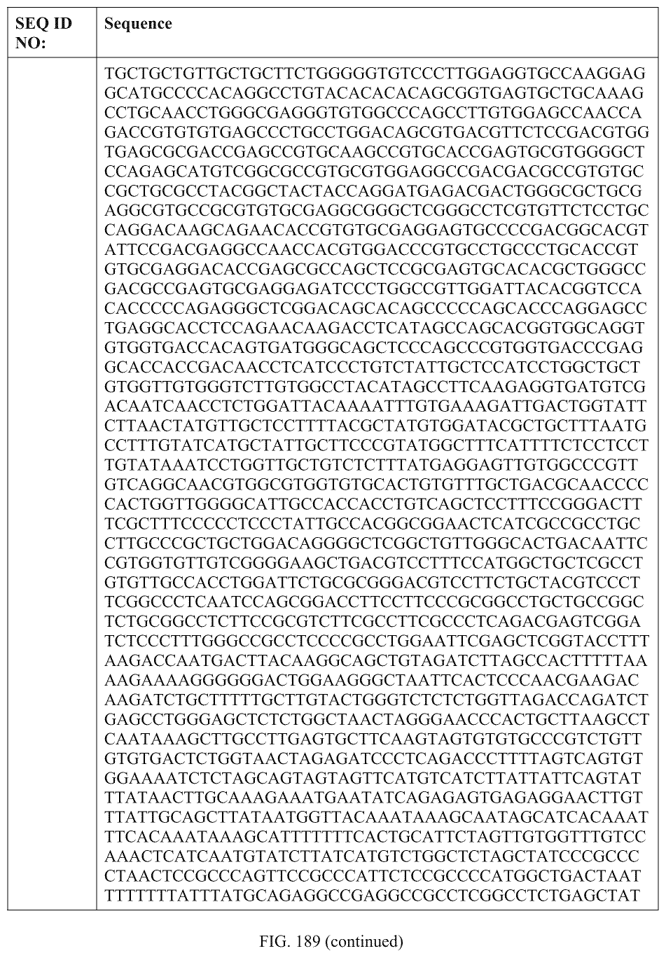

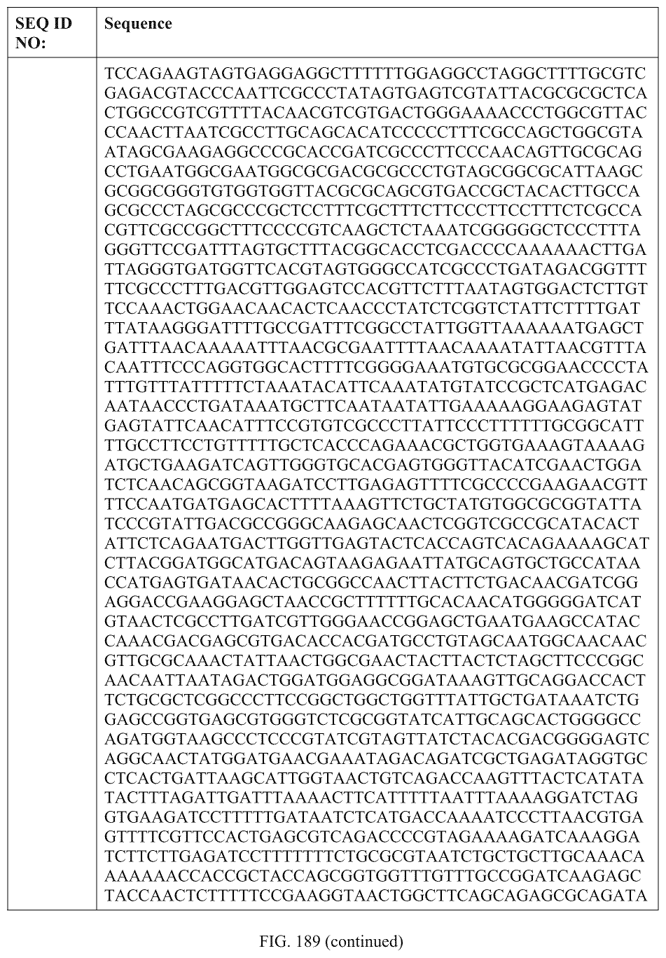

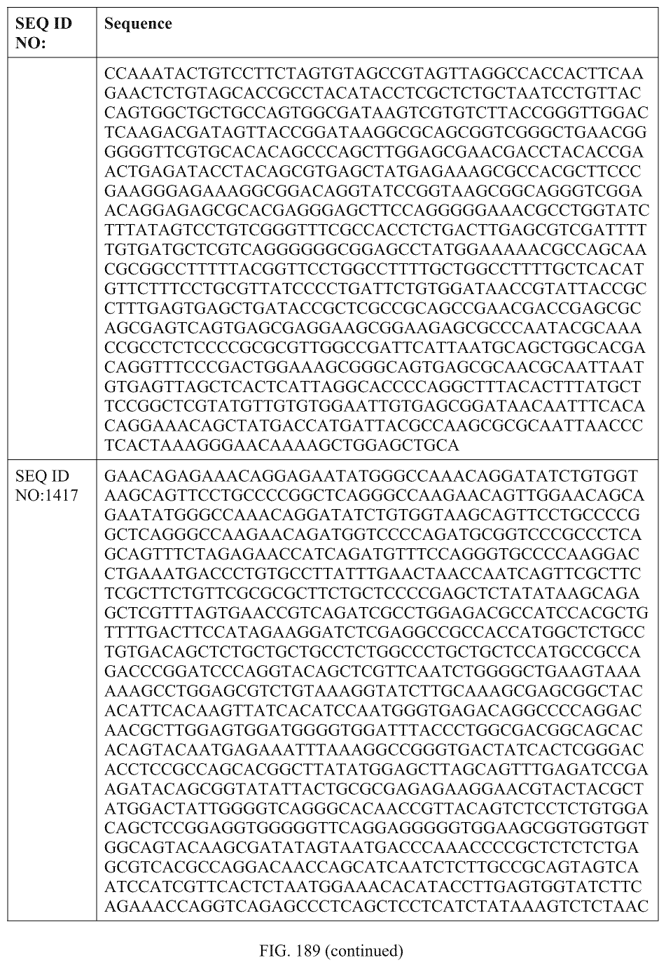

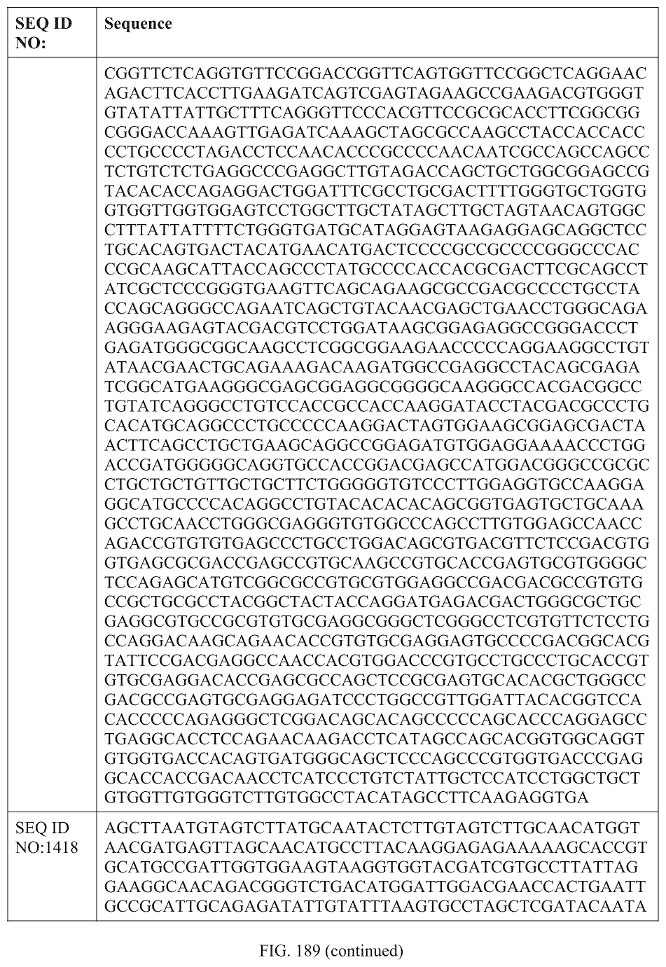

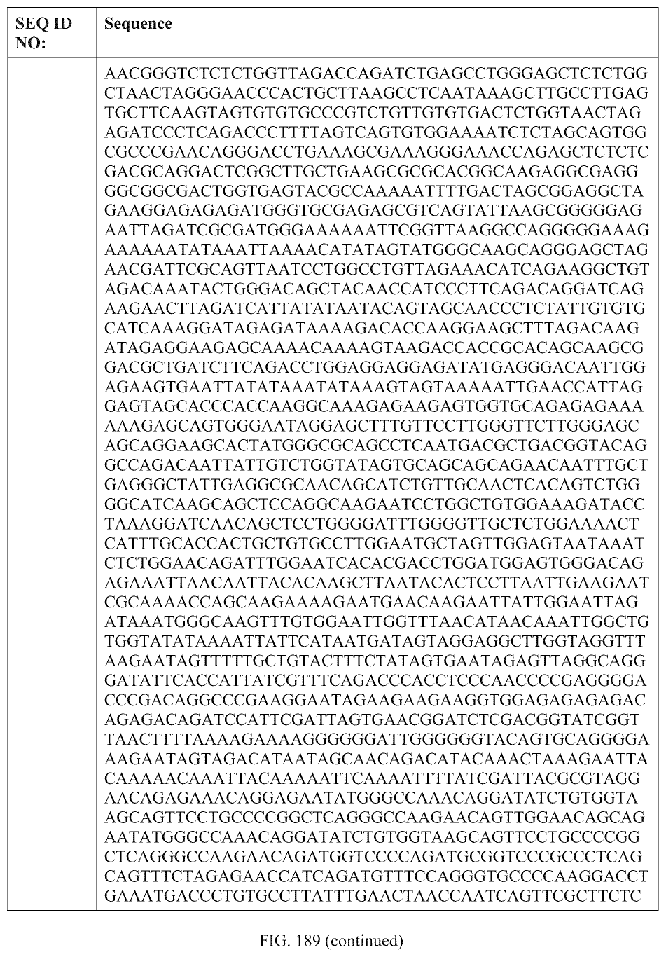

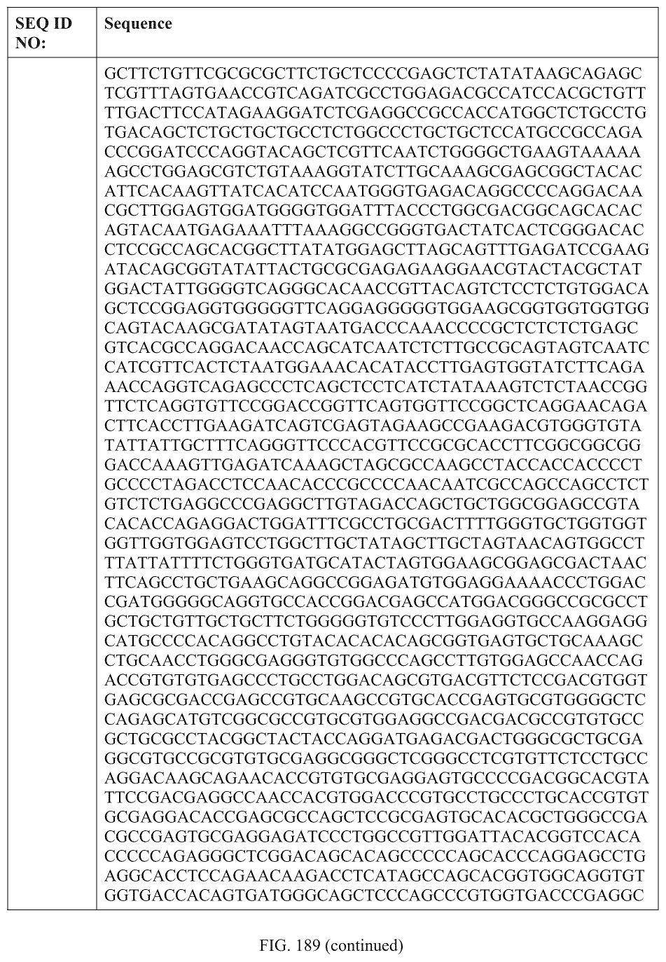

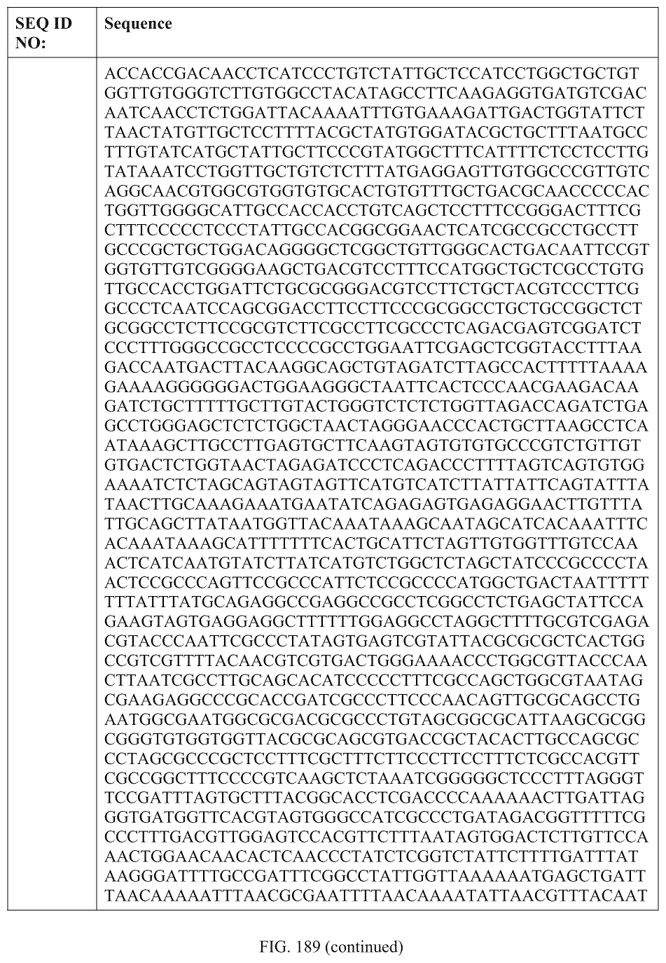

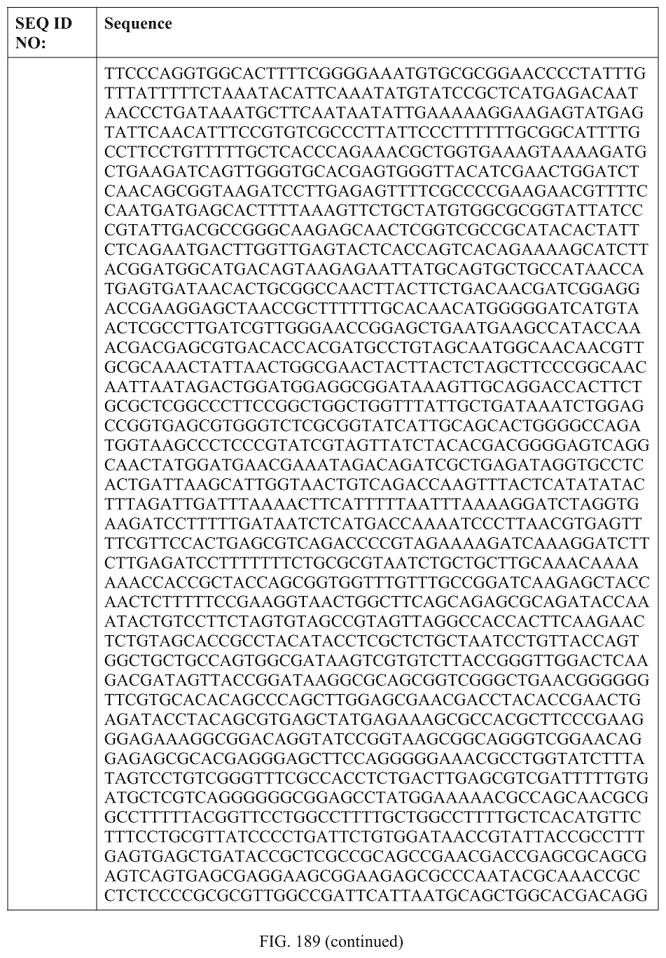

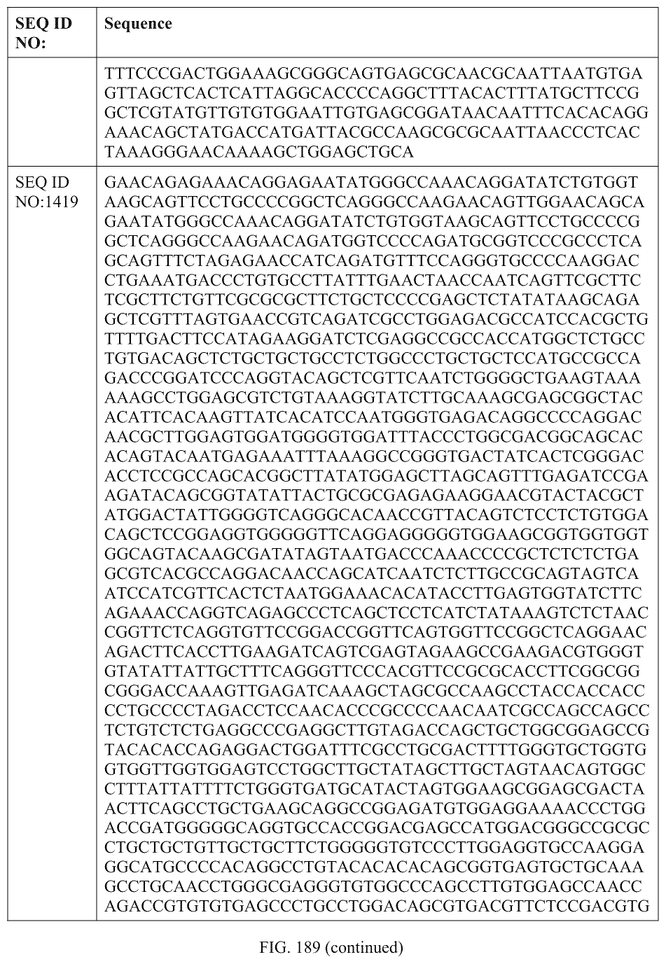

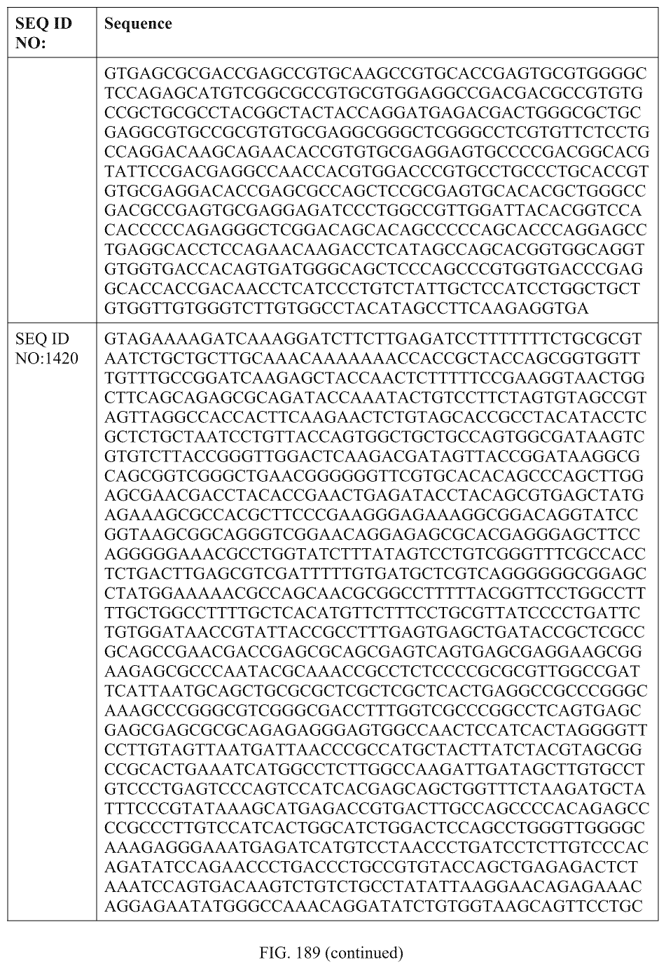

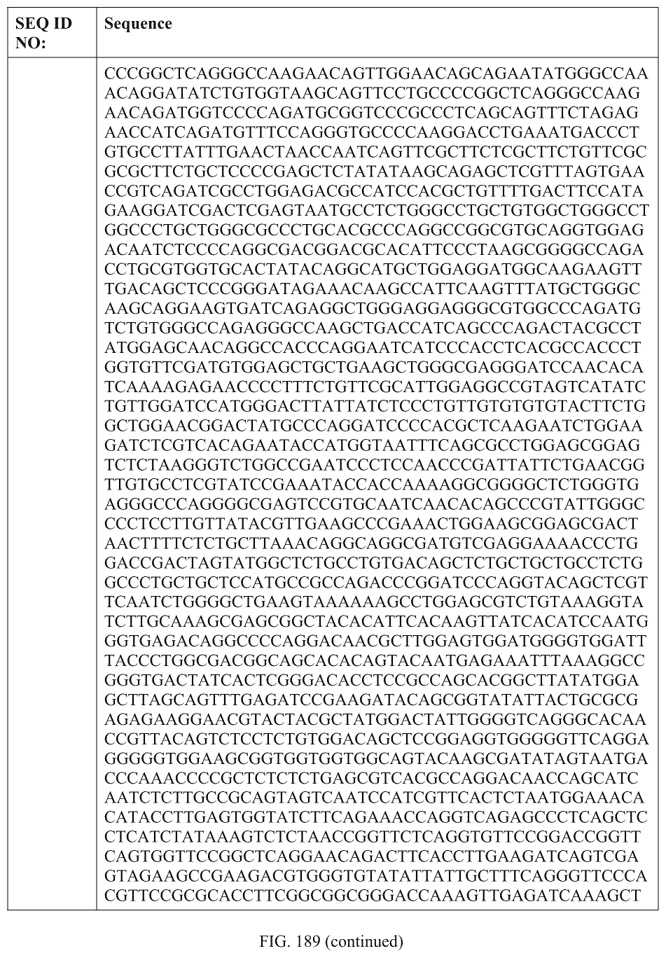

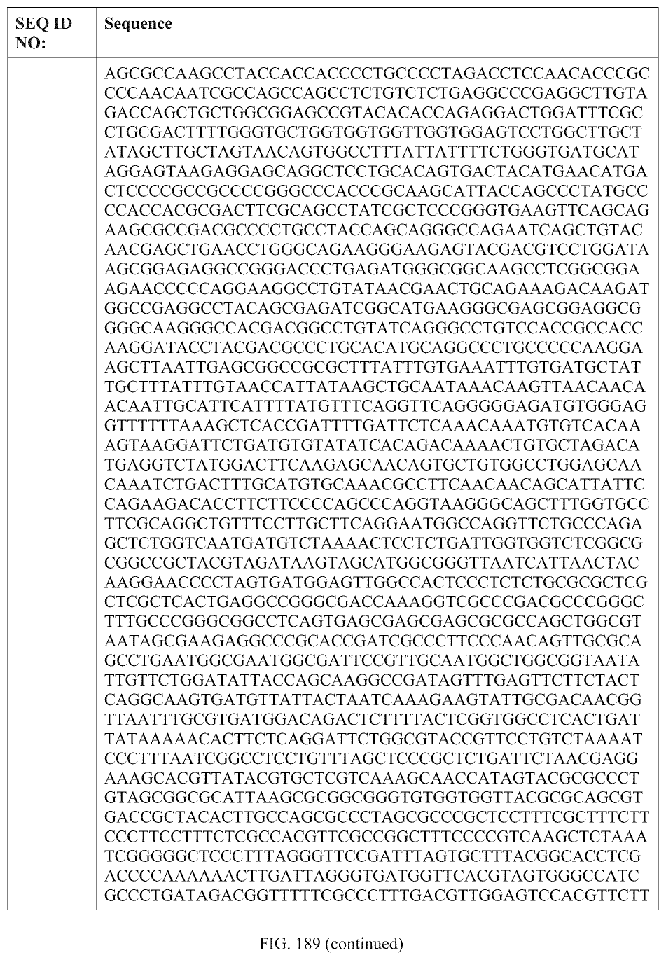

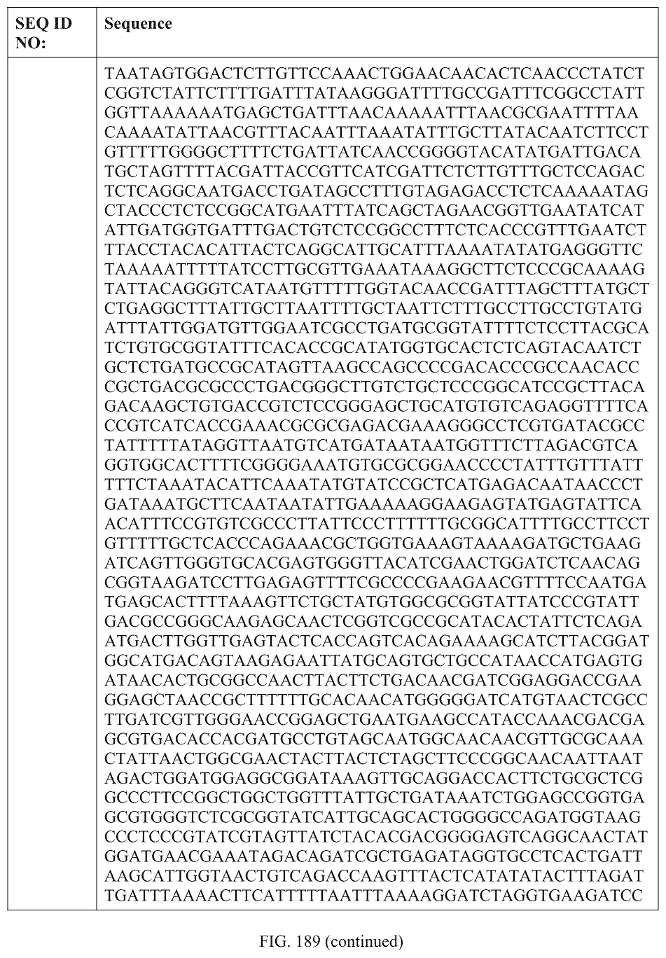

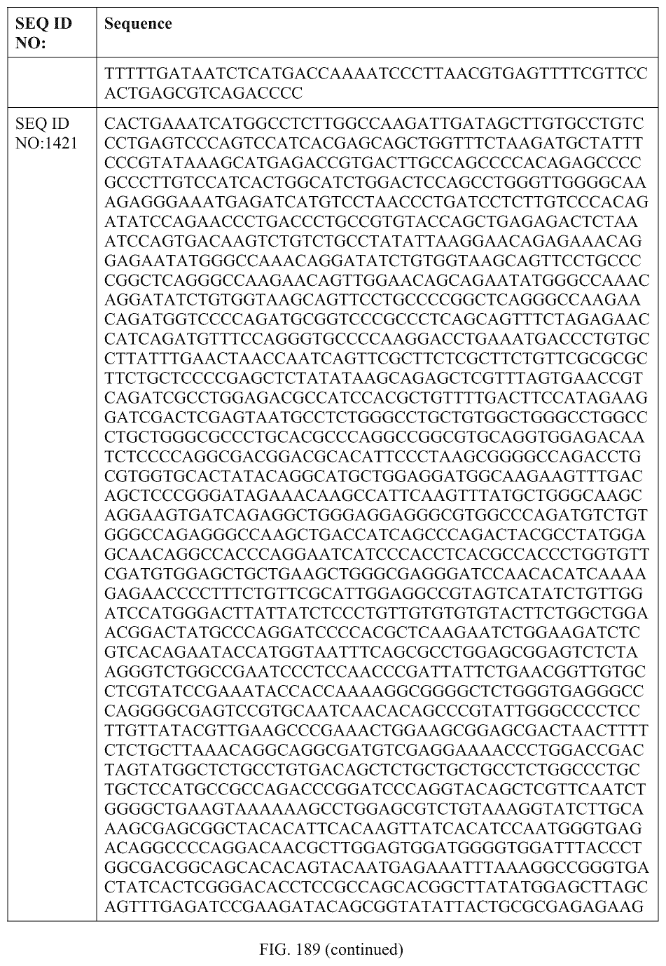

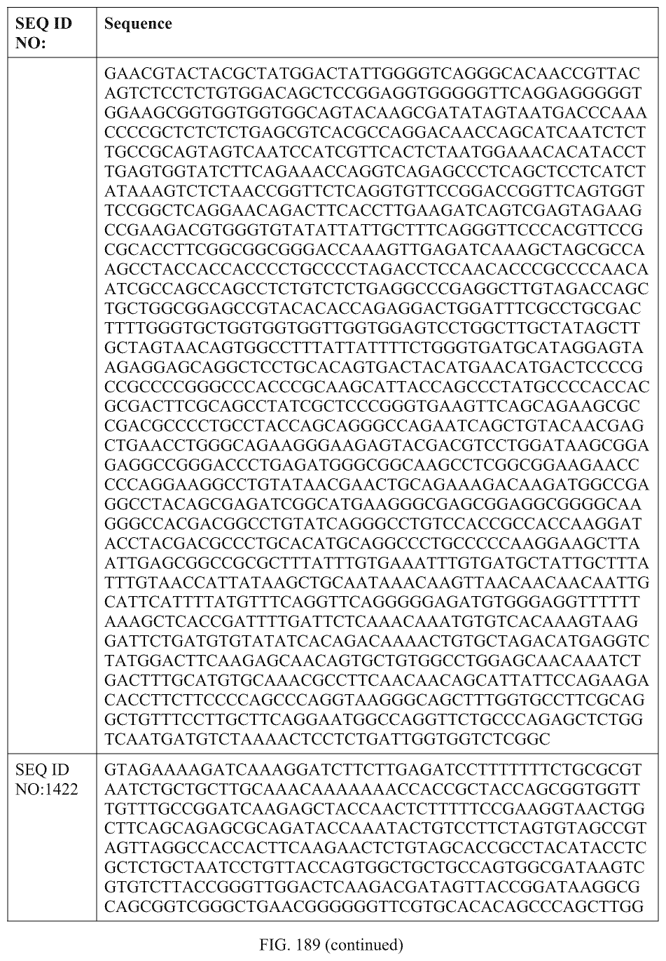

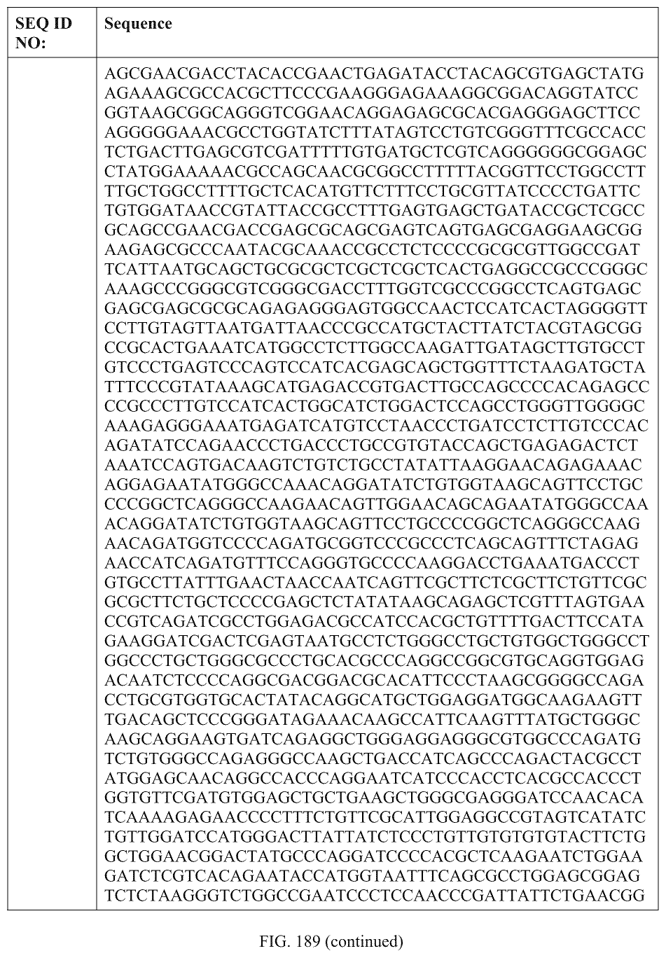

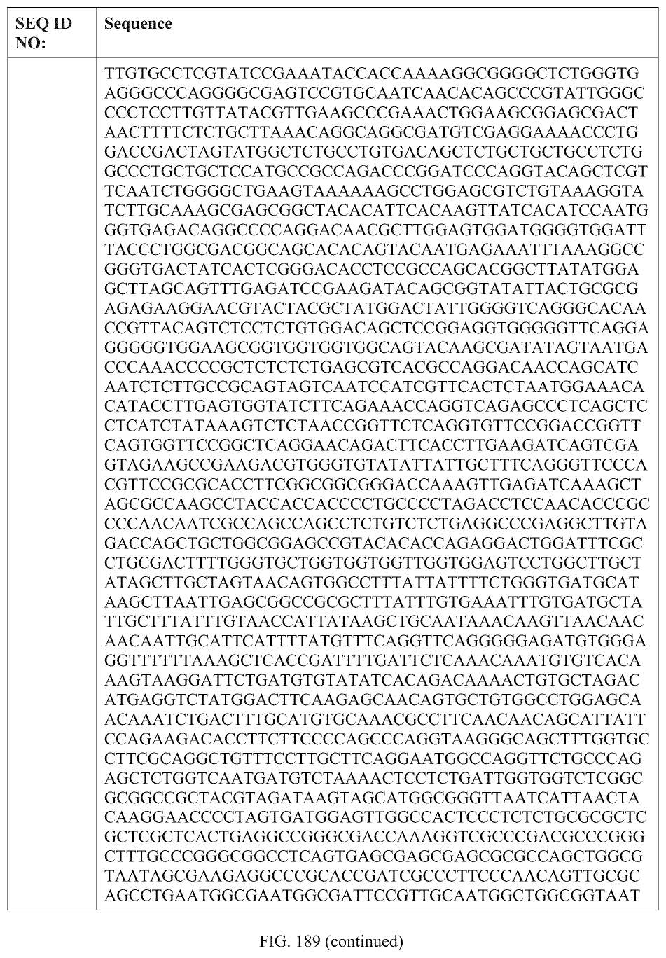

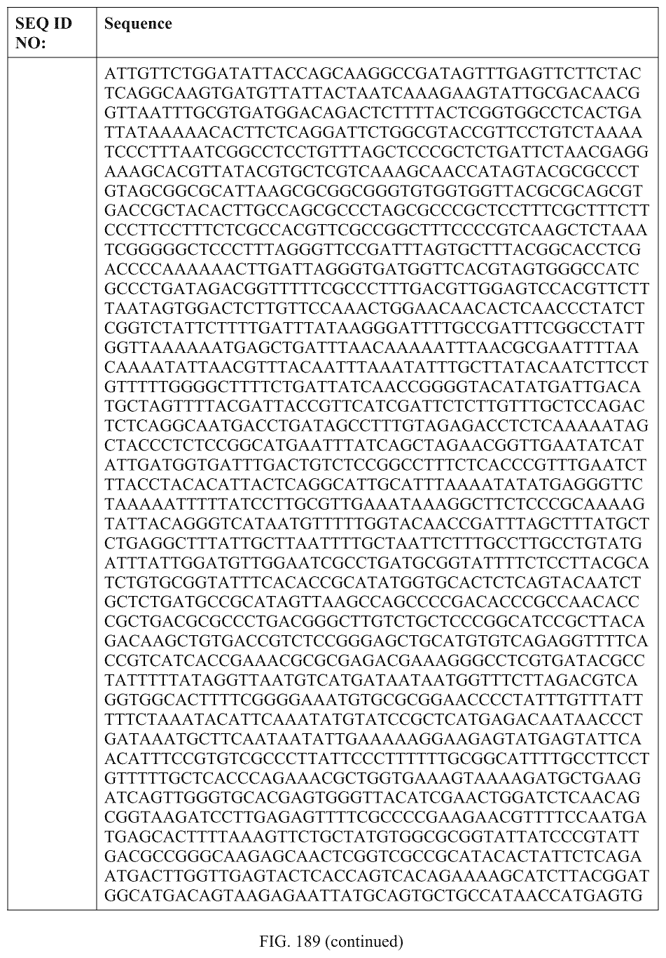

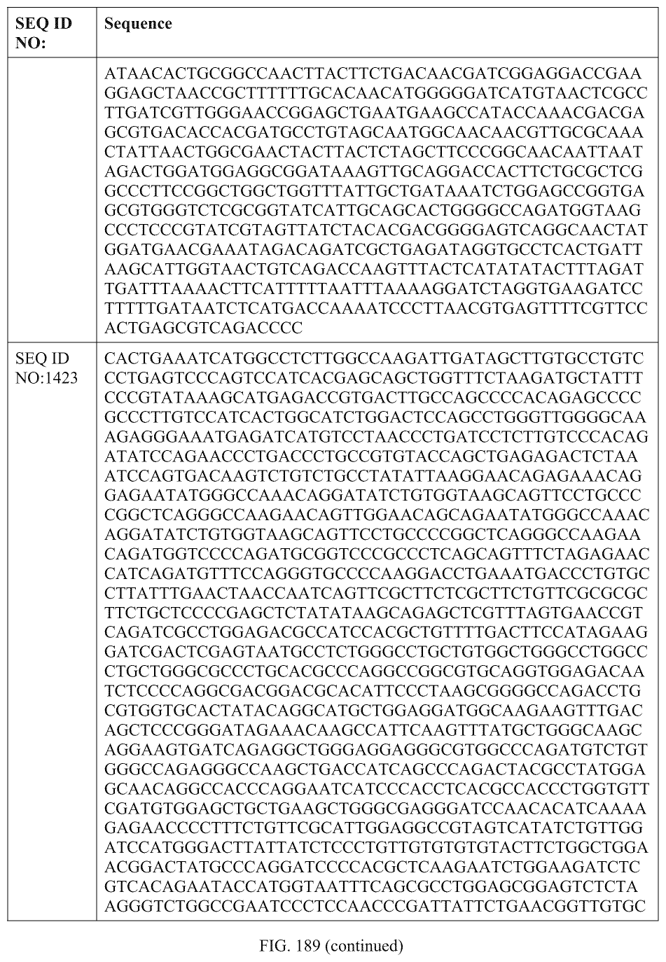

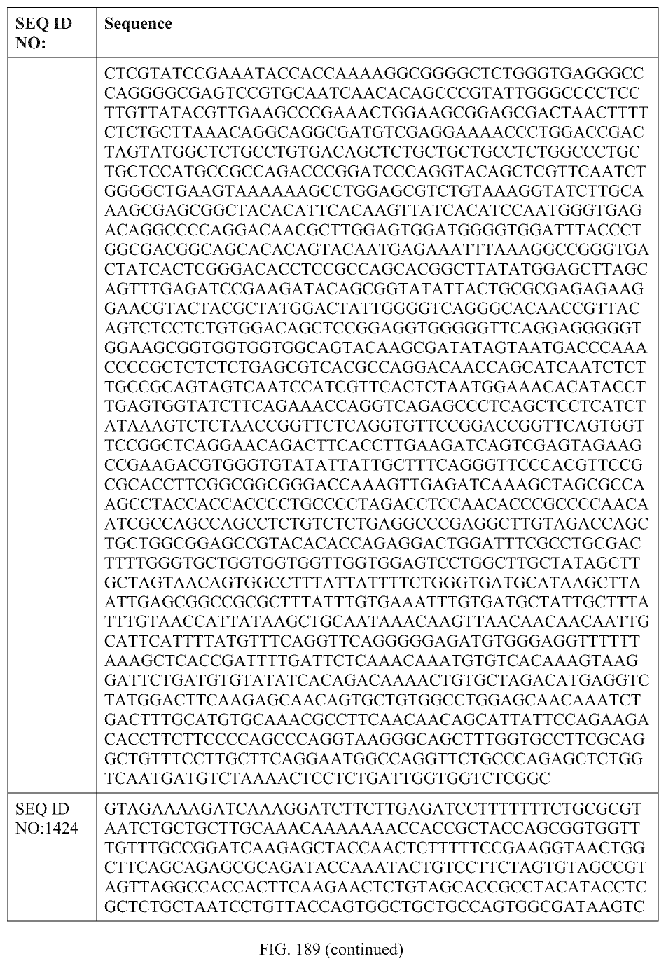

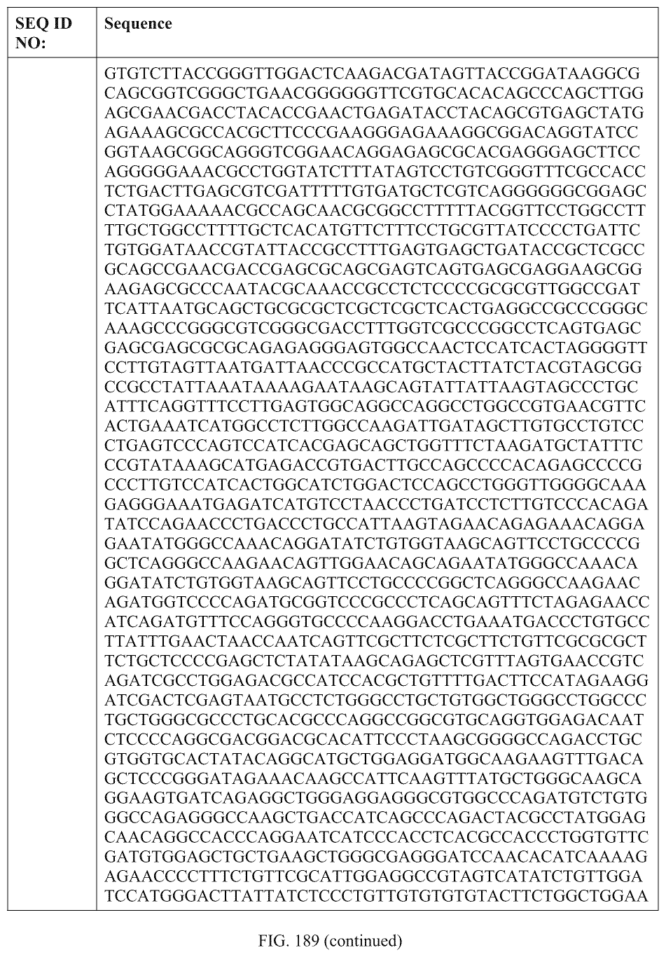

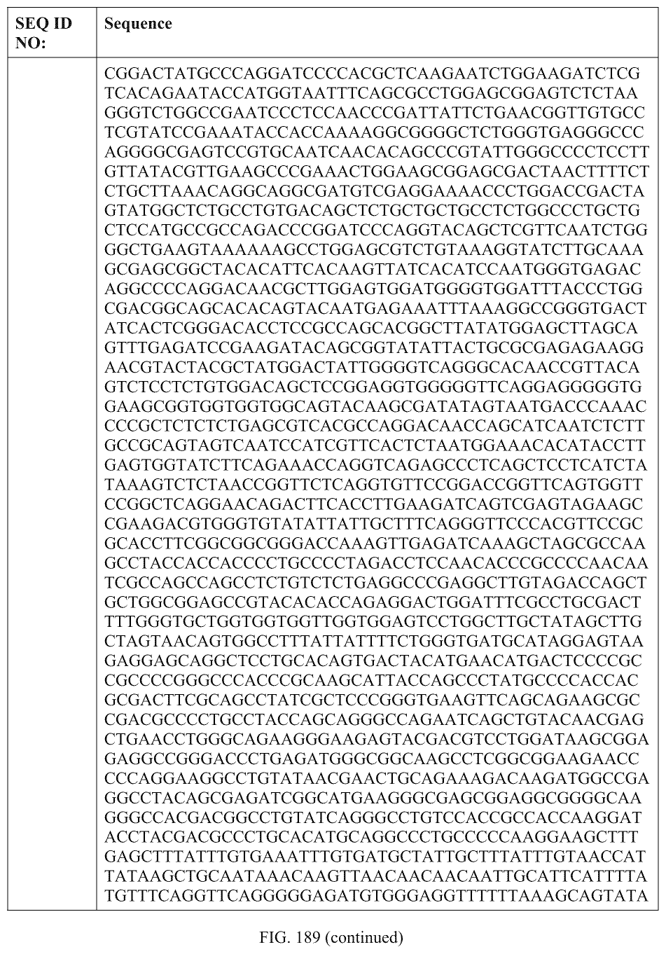

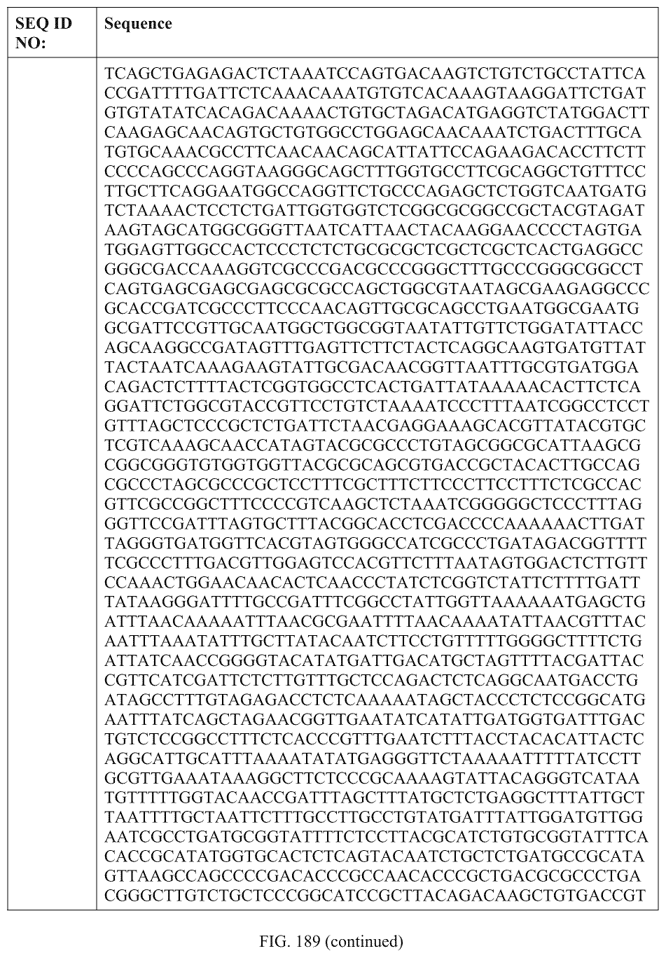

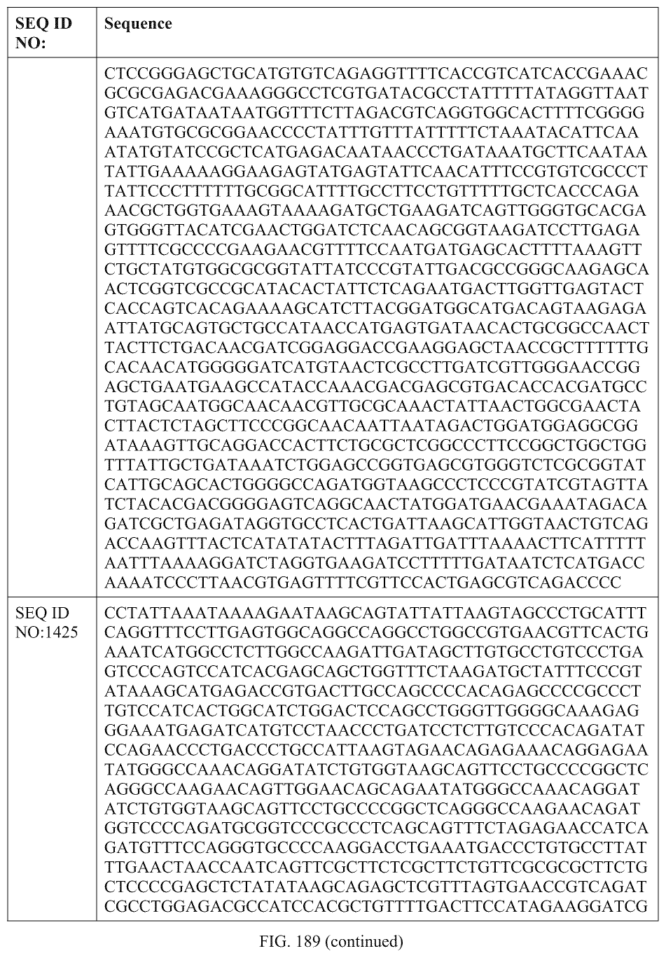

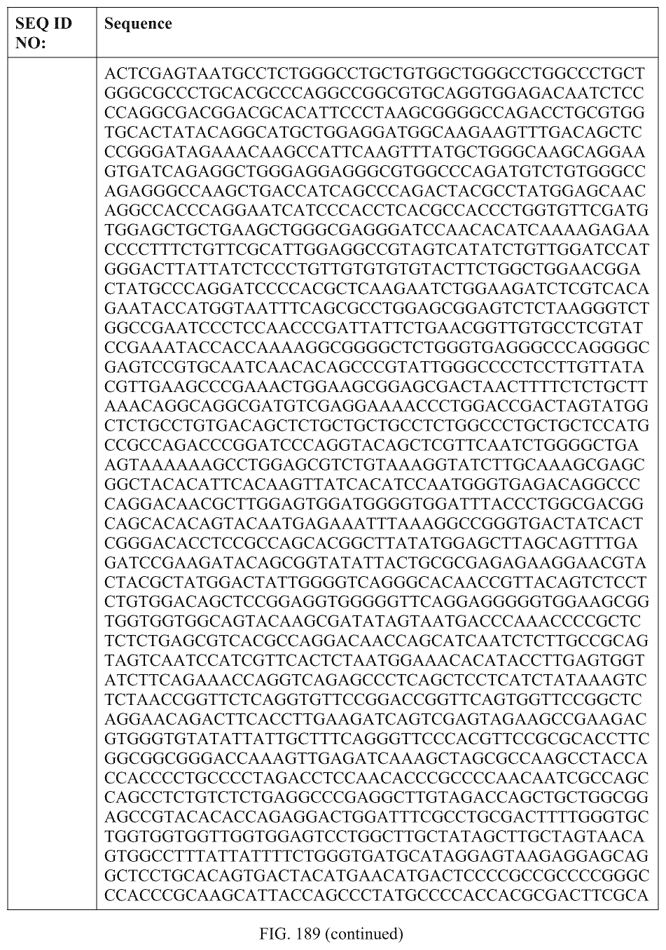

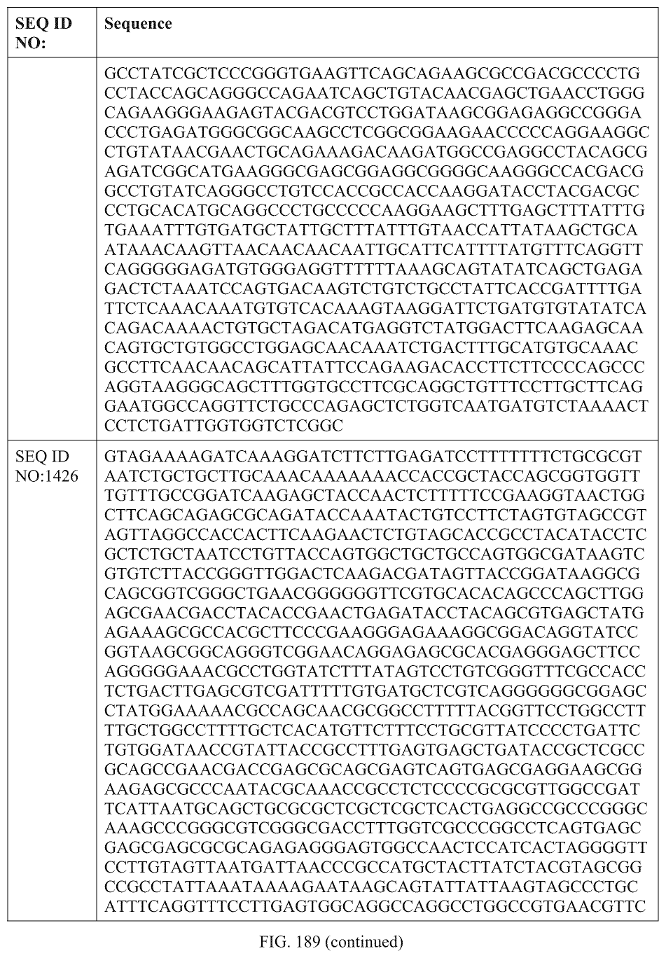

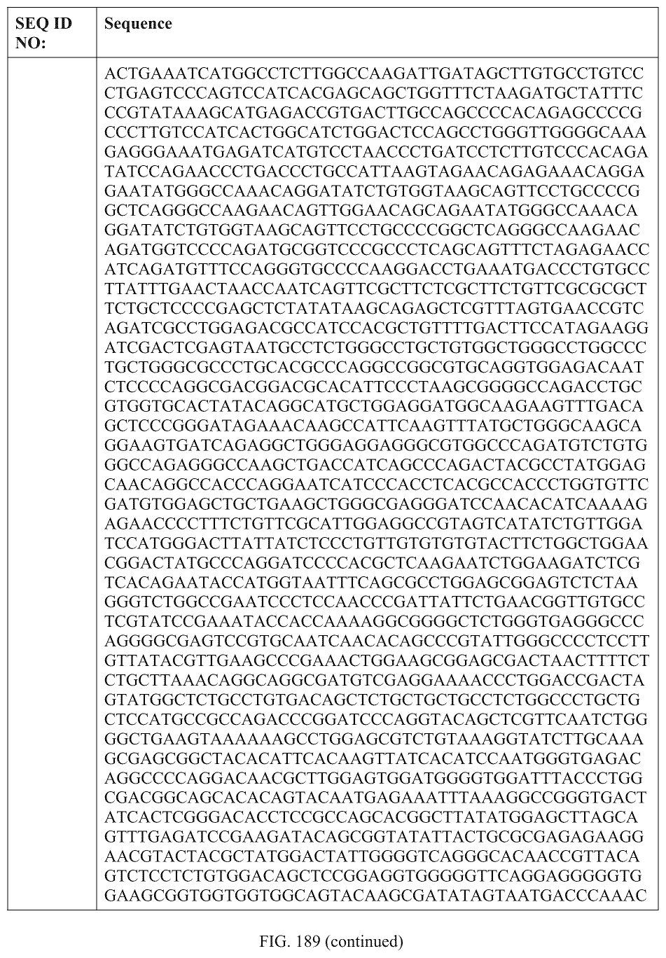

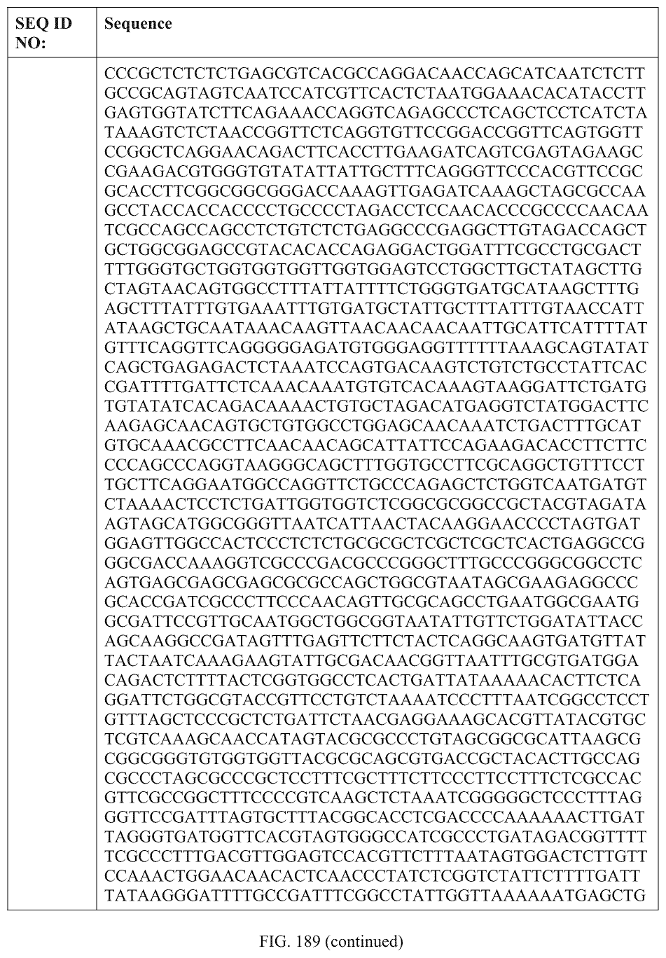

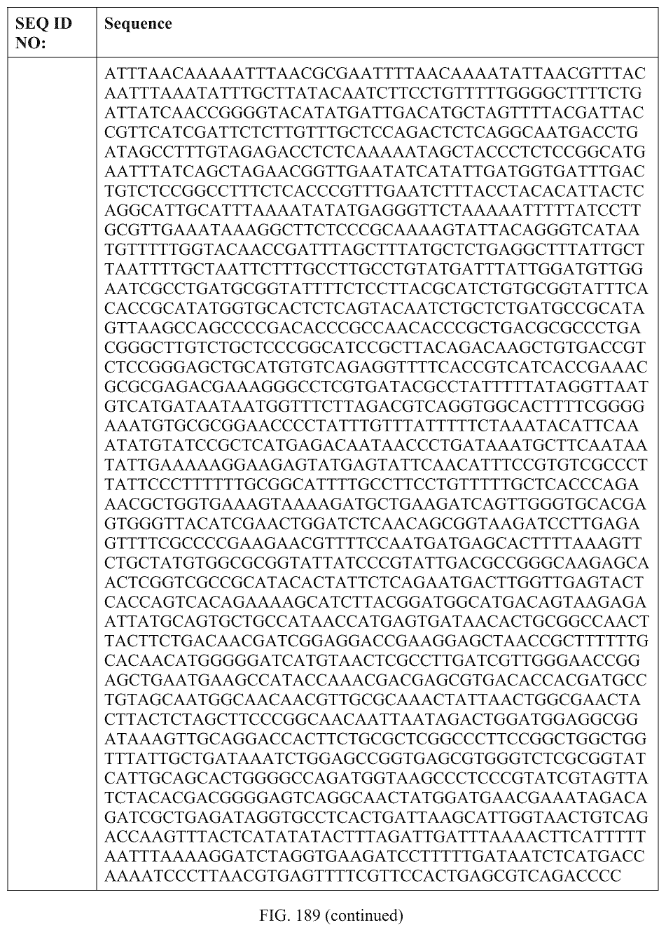

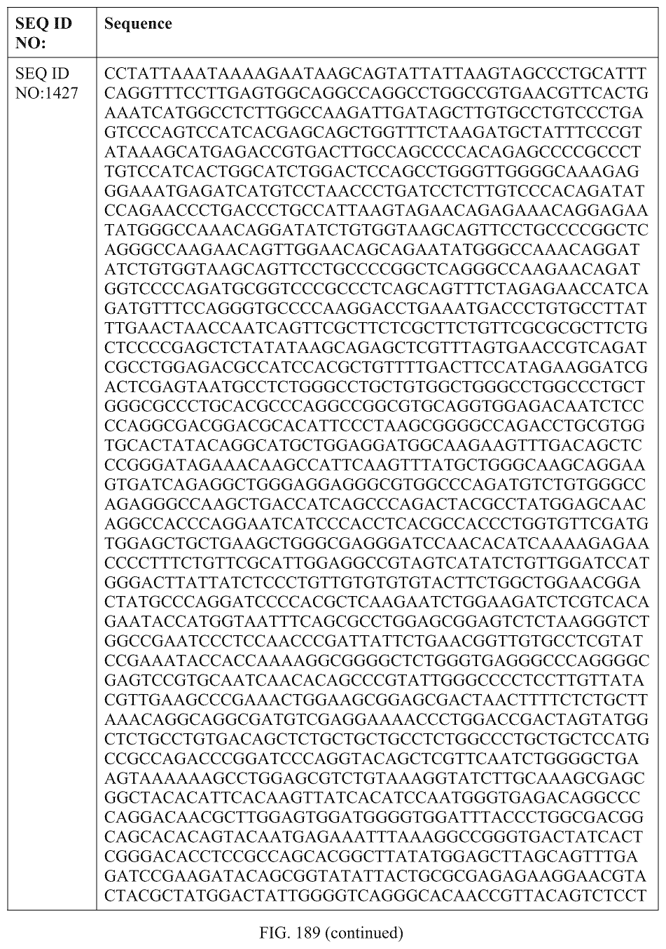

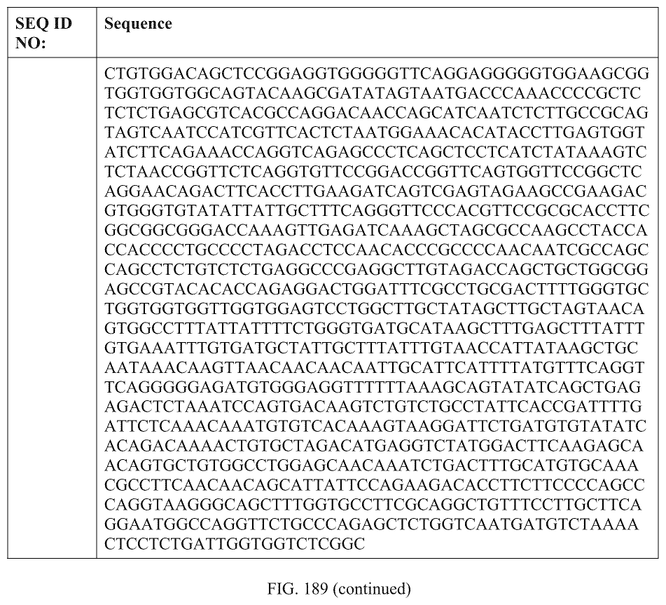

FIGS. 180A-180D depict timelines, editing constructs and cell products produced using LV.A2CAR+single edit and dual editing strategies. Samples 1-6 include FOXP3 locus single edited cells with or without (+/−) LV A2CAR transduction in CD3+ and CD8+ cells. This approach was used to generate mock, polyclonal CISC EngTregs and LV.A2CAR CISC EngTregs. Samples 7-9 include dual-edited CD3 T cells. Sample 7 is a control. Sample 8 includes dual-edited A2CAR Split-CISC EngTregs. CD3 T cells were treated with T9 RNP targeting FOXP3 T9 and T4 RNP targeting TRAC plus AAV 3363 and AAV 3407. Sample 9 was first transduced with LV.A2CAR and then dual edited using FOXP3 T9 and TRAC T4 RNP plus AAV 3195 generating LV.A2CAR-TCRnull-CISC EngTregs. Certain constructs encoding A2 CARs are listed in the following TABLE 4. Sequences are shown in FIG. 189.

TABLE 4

| Construct | Label | SEQ ID NO: |
|---|---|---|
| VECTOR: lentiviral vector expressing | LV.3350_pRRL_{—} | SEQ ID NO: 1416 |
| A2CAR.P2A and truncated LNGFR | MND.A2scFV(H1K2).CD28TM.CD28.CD3z- | |
| | P2A.LNGFR | |
| extracted sequence from LVA2CAR (3350_pRRL_{—} | | SEQ ID NO: 1417 |
| MND.A2scFV(H1K2).CD28TM.CD28.CD3z- | | |
| P2A.LNGFR) containing the gene | | |
| expression cassette: | | |
| MND.A2scFV.CD28TM.CD28.CD3z-P2A.LNGFR | | |
| VECTOR: lentiviral vector expressing a non- | LV.delta A2CAR | SEQ ID NO: 1418 |
| signaling A2CAR protein | (3351_pRRL_MND.A2scFV(H1K2).CD28TM- | |
| | P2A.LNGFR), non-signaling | |
| extracted sequence from LV.delta A2CAR | | SEQ ID NO: 1419 |
| (3351_pRRL_MND.A2scFV(H1K2).CD28TM- | | |
| P2A.LNGFR) for the gene expressing cassette | | |
| containing MND.A2scFV(H1K2).CD28TM- | | |
| P2A.LNGFR) | | |
| VECTOR: TRAC-T4 guide specific AAV, with | AAV A2CAR for TRAC locus | SEQ ID NO: 1420 |
| 0.3 kb homology arms upstream and | (3407_AAV.TRAC03_{—} | |
| downstream of gene expression cassette | MND.FKBP.IL2Rg.P2A.A2CAR.polyA_{—} | |
| containing the MND promoter, FKBP.IL2Rg | 0.3_T4) | |
| coding sequence, P2A coding sequence, | | |
| A2CAR coding sequence and poly A signal | | |
| extracted sequence from 3407 containing 0.3 | | SEQ ID NO: 1421 |
| kb 5′ homology arm, the MND promoter, | | |
| FKBP.IL2Rg coding sequence, P2A coding | | |
| sequence, A2CAR coding sequence, poly A | | |
| signal, and 0.3 kb 3′ homology arm | | |
| VECTOR: TRAC-T4 guide specific AAV, with | AAV delta A2CAR for TRAC locus | SEQ ID NO: 1422 |
| 0.3 kb homology arms upstream and | (3408_AAV.TRAC03_{—} | |
| downstream of gene expression cassette | MND.FKBP.IL2Rg.P2A.A2CAR.polyA_{—} | |
| containing the MND promoter, FKBP.IL2Rg | 0.3_T4), non-signaling | |
| coding sequence, P2A coding sequence, non- | | |
| signaling A2CAR coding sequence and poly A | | |
| signal | | |
| extracted sequence from 3408 containing 0.3 | | SEQ ID NO: 1423 |
| kb 5′ homology arm, the MND promoter, | | |
| FKBP.IL2Rg coding sequence, P2A coding | | |
| sequence, non-signaling A2CAR coding | | |
| sequence, poly A signal, and 0.3 kb 3′ | | |
| homology arm | | |
| VECTOR: TRAC-T1 guide specific AAV, with | AAV A2CAR for TRAC locus | SEQ ID NO: 1424 |
| 0.3 kb homology arms upstream and | (3409_TRAC03_{—} | |
| downstream of gene expression cassette | MND.FKBP.IL2Rg.P2A.A2CAR.polyA | |
| containing the MND promoter, FKBP.IL2Rg | for T1) | |
| coding sequence, P2A coding sequence, | | |
| A2CAR coding sequence and poly A signal | | |
| extracted sequence from 3409 containing 0.3 | | SEQ ID NO: 1425 |
| kb 5′ homology arm, the MND promoter, | | |
| FKBP.IL2Rg coding sequence, P2A coding | | |
| sequence, A2CAR coding sequence, poly A | | |
| signal, and 0.3 kb 3′ homology arm | | |
| VECTOR: TRAC-T1 guide specific AAV, with | AAV deltaA2CAR for TRAC locus | SEQ ID NO: 1426 |
| 0.3 kb homology arms upstream and | (3410_TRAC03_{—} | |
| downstream of gene expression cassette | MND.FKBP.IL2Rg.P2A.A2CARdelta.polyA | |
| containing the MND promoter, FKBP.IL2Rg | for T1, non-signaling | |
| coding sequence, P2A coding sequence, non- | | |
| signaling A2CAR coding sequence and poly A | | |
| signal | | |
| extracted sequence from 3410 containing 0.3 | | SEQ ID NO: 1427 |
| kb 5′ homology arm, the MND promoter, | | |
| FKBP.IL2Rg coding sequence, P2A coding | | |
| sequence, non-signaling A2CAR coding | | |
| sequence, poly A signal, and 0.3 kb 3′ | | |
| homology arm | | |

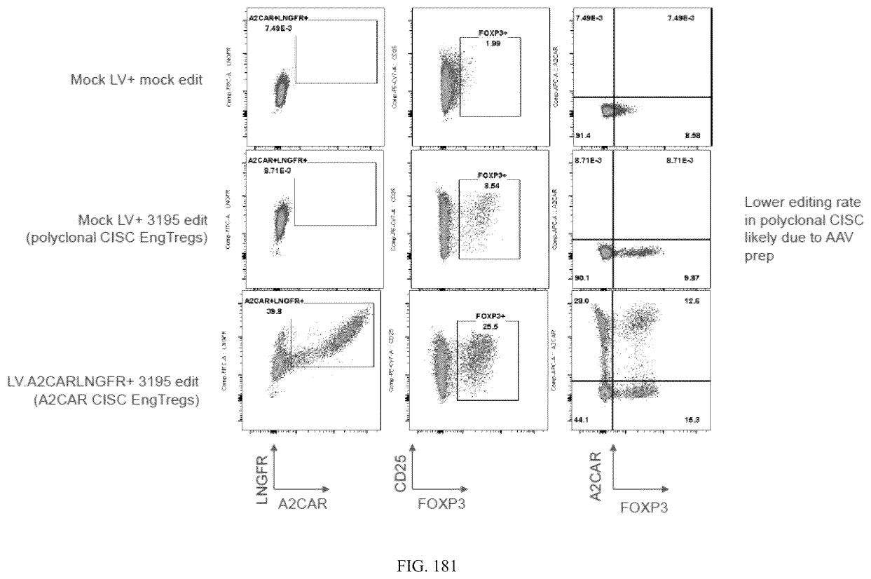

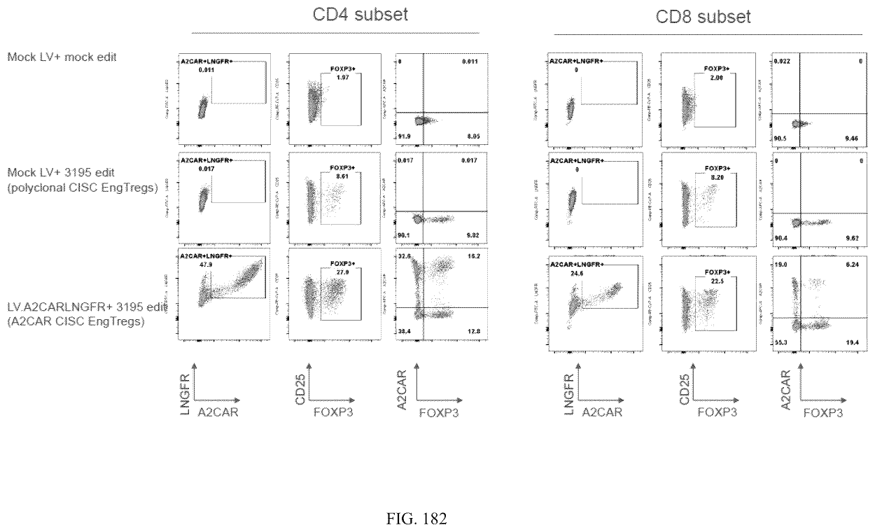

All the samples were analyzed on day 3 post editing for HIDR (FIG. 181-FIG. 184) and showed moderate to high levels of single and dual editing. HIDR in CD3 cells was detected by flow on day 3 post editing and demonstrated dual expression of FOXP3 and A2CAR in LV.A2CAR CISC EngTregs in 12.600 of the cells (FIG. 181). Looking at individual subsets within CD3 T cells, it was demonstrated that the cells included 16.2% A2CAR+/FOXP3+ cells in the CD4+ subset and 6.2% A2CAR+/FOXP+ in the CD8+ subset compared to 0% for mock and polyclonal CISC EngTregs (FIG. 182).

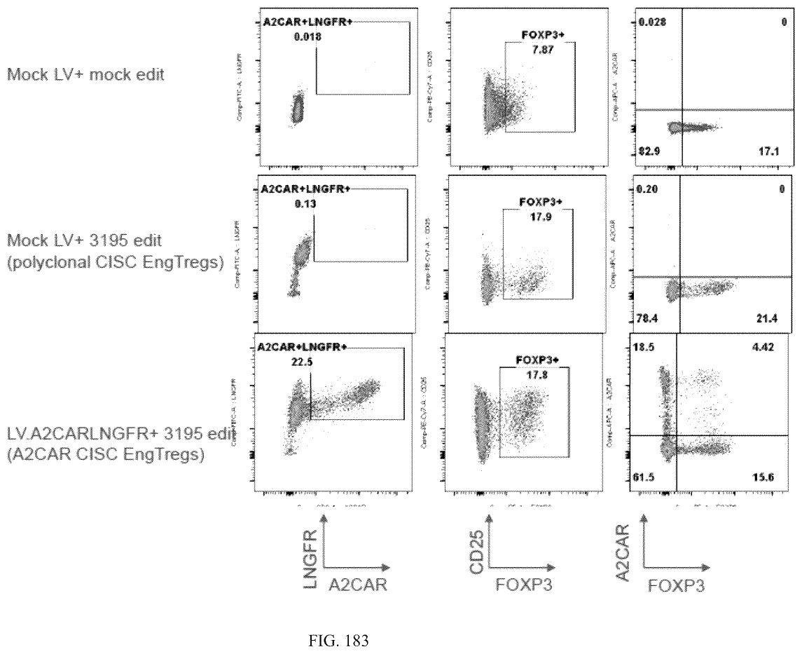

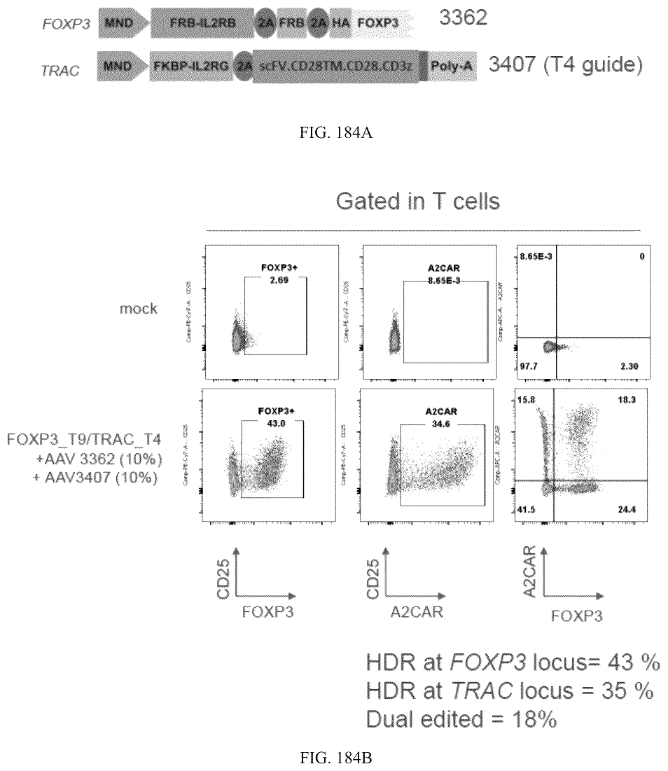

HDR analysis in CD8 cells showed 4.42% of the cells had dual expression of FOXP3 and A2CAR in the LV.A2CAR CISC EngTregs (FIG. 183). FIG. 184B shows the flow-based HDR detection at day 3 post-editing in mock and dual-editing CD3 cells (mock vs A2CAR Split-CISC CD3 EngTregs) demonstrating high single and dual editing efficiency. HDR was 43% and 35% at the FOXP3 and TRAC locus respectively. Strikingly, 18.3% of the dual HDR-edited cells expressed both A2CAR and FOXP3+.

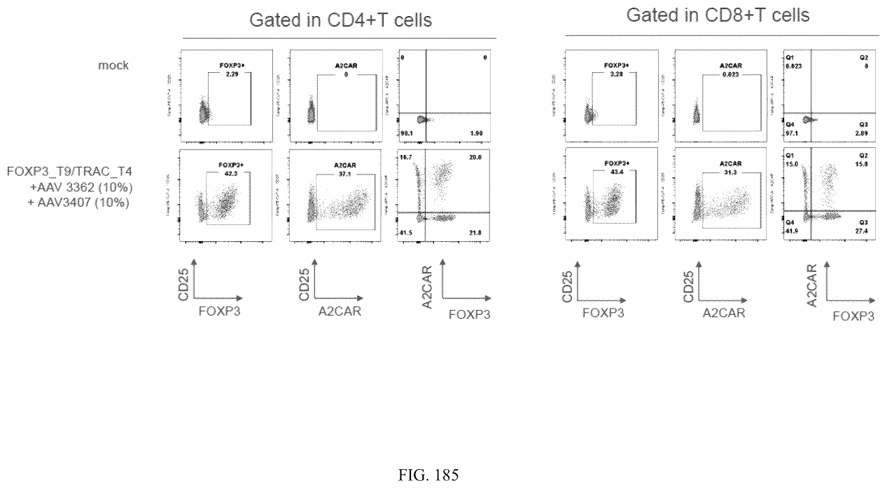

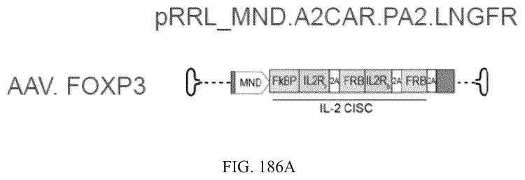

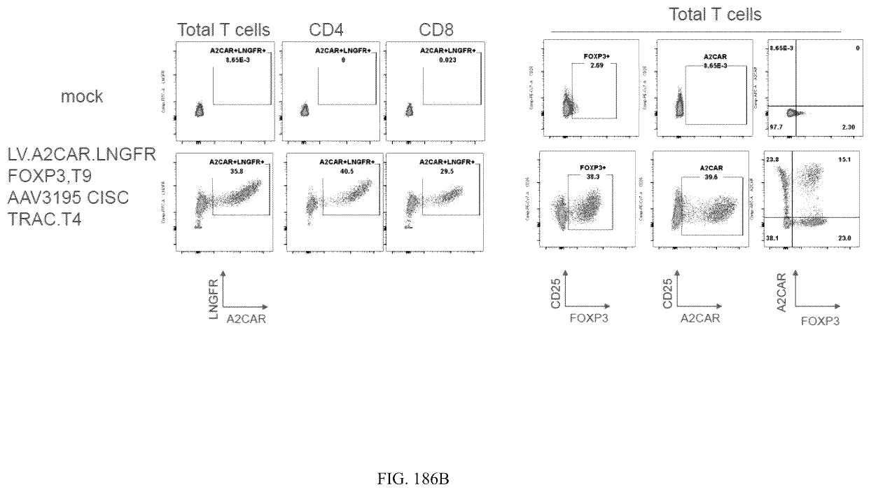

Notably, CD4+ and CD8+ subsets of the CD3 dual-edited cells showed a similar single and dual editing rates (42.3% and 43.4% in CD4+ gated and CD8+ gated cells respectively for at FOXP3 locus and 37.1% and 31.3% in CD4+ gated and CD8+ gated cells for TRAC locus) FIG. 185. Dual-edited cells expressing A2CAR and FOXP3 was 20% for CD4 and 15.8% for CD8 T cell subsets respectively. In addition, LVA2CAR transduced CD3+ T cells treated with FOXP3 T9 and TRAC T4 RNP followed by AAV 3195 (CISC donor template), to generate LV.A2CAR-TCRnull-CISC CD3+ EngTreg cell product has 15.1% A2CAR+/FOXP+ cells (FIG. 186A, FIG. 186B).

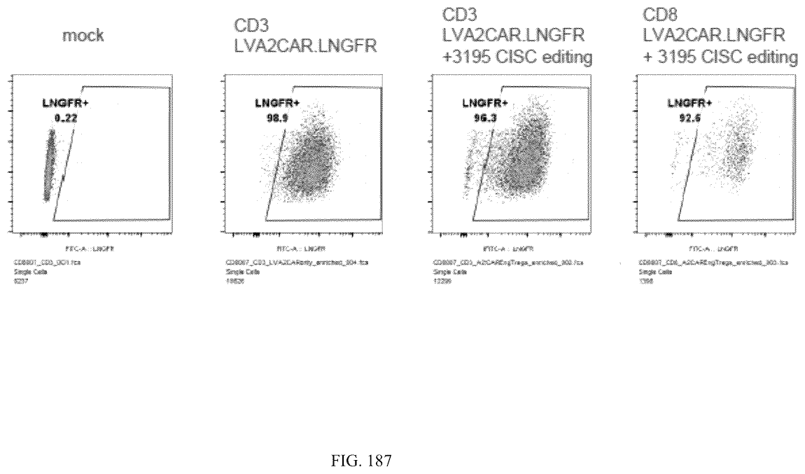

Editing using full CISC constructs also included selective enrichment of HDR edited CD8 EngTregs. Two to three days post editing, prior to enrichment with rapamycin, cells transduced with LV.A2CAR were LNGFR enriched with LNGFR affinity selection. The cell purity, verified by flow, ranged from 92.6% to 98.9% purity in CD3 LVA2CAR.LNGFR, CD3 LVA2CAR.LNGFR+3195 CISC editing and CD8 LVA2CAR.LNGFR+3195 CISC edited cells (FIG. 187). Cells with full CISC constructs and split CISC constructs were expanded in rapamycin and were analyzed for expansion, phenotype, cytokine and in vitro suppression.

In summary, an analysis of CD8+ EngTregs was extended (derived from CISC EngTreg CD3+ T cells) to include a demonstration of suppressive activity equal to or greater than what is observed with CD4+ EngTregs. It was also demonstrated that generate CD8+ EngTregs can be generated with a T regulatory phenotype by editing purified CD8+ T cells. Third, importantly, a dual editing approach was used successfully to generate an antigen-specific CISC-expressing dual-edited CD8+ EngTreg (A2CAR Split-CISC EngTreg) from CD3+ T cells that expresses A2CAR, FOXP3 and split CISC. These cells are currently expanding, and it is anticipated that dual-edited A2CAR-specific CD8 EngTregs will enrich in rapamycin and exhibit T reg characteristics. Future studies using these cell products will include an assessment of the antigen-specific CISC-expressing dual-edited CD8+ EngTreg in comparison to a similar CD4+ cell product to evaluate suppressive function in vitro and in a GVHD in vivo model.

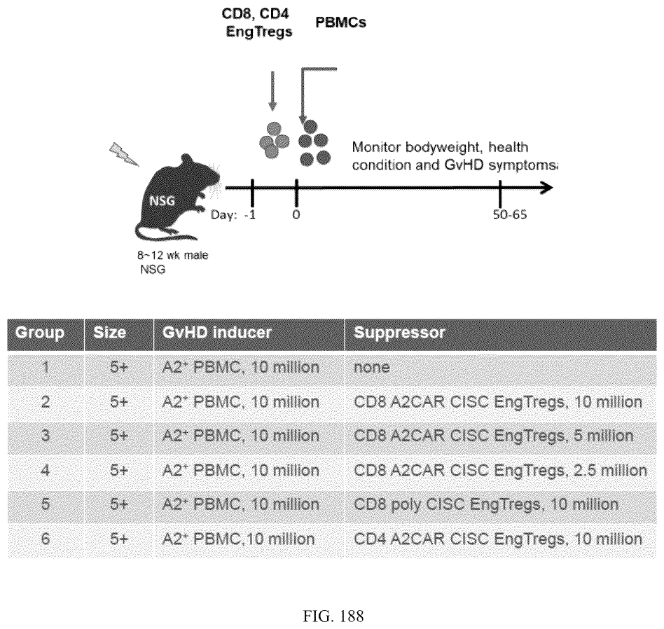

To test the efficacy of CD8 A2CAR CISC EngTregs compared to CD4 A2CAR CISC EngTregs in an in vivo model, a GVHD in vivo study is performed. This study uses A2+ PBMCs as effector cells to induce GVHD in NSG mice in the presence of CD8 or CD4 EngTregs. FIG. 188 depicts an outline of a study plan. CD4 and CD8 A2CAR EngTregs both reduce GVHD in the subjects, and CD8 EngTregs perform better in this model.

### Example 23—Islet-Specific Engineered Treg Exhibit Robust Antigen-Specific and Bystander Immune Suppression in Type 1 Diabetes Models

Adoptive transfer of regulatory T cells (Treg) is therapeutic in Type 1 diabetes (T1D) mouse models. Notably, Treg specific for pancreatic islets are more potent than polyclonal Treg in preventing disease. However, the frequency of antigen-specific natural Treg is extremely low and ex vivo expansion may destabilize Treg leading to an effector phenotype. Disclosed herein are durable, antigen-specific engineered (Eng) Treg derived from primary human CD4+ T cells by combining FOXP3 homology-directed repair editing and lentiviral TCR delivery. Using TCRs from clonally expanded CD4+ T cells in T1D, islet-specific EngTregs that suppressed effector T cell (Teff) proliferation and cytokine production were generated. EngTregs suppressed Teff recognizing the same islet antigen as well as bystander Teff recognizing other islet antigens via production of soluble mediators and both direct and indirect mechanisms. Adoptively transferred murine islet-specific EngTregs homed to the pancreas and blocked diabetes triggered by islet-specific Teff in recipient mice. These data demonstrated the use of antigen-specific EngTregs as a targeted therapy to treat or prevent TID.

T1D is an organ-specific autoimmune disease where autoreactive T cells target insulin-producing beta cells in the pancreatic islets resulting in a severe loss of endogenous insulin production (1, 2). Regulatory T cells (Treg), characterized by expression of the forkhead box transcription factor FOXP3, are important for maintaining peripheral tolerance and preventing excessive immune responses and autoimmunity. In humans, loss-of-function mutations in the FOXP3 gene leads to Treg defects resulting in a severe multi-organ autoimmune and inflammatory disorder referred to as immune dysfunction, polyendocrinopathy, enteropathy, X-linked (IPEX) syndrome. Among the wide range of autoimmune disorders in IPEX is early onset of T1D, demonstrating a key role of FOXP3⁺ Treg in maintaining islet-specific tolerance (1, 3). Several studies have suggested that reduced Treg number or impaired Treg function could be central to the pathogenesis of T1D (1, 2, 4, 5). Consequently, increasing the number or functional activity of Treg has become a major candidate strategy for therapeutic intervention to treat and prevent the disease (6, 7).

The therapeutic potential of Treg has been shown in various preclinical models of organ transplantation and autoimmune diseases (8). While adoptive transfer of expanded polyclonal Treg has shown clinical activity (8), it has been demonstrated that antigen-specific Treg are more efficacious than polyclonal Treg in numerous preclinical studies including T1D, multiple sclerosis, colitis, rheumatoid arthritis, and transplantation (9-15). For example, Treg specific for pancreatic islet antigens were more effective than polyclonal Treg in preventing T1D progression in murine models of T1D, and even reversed disease (9, 16, 17). Moreover, polyclonal Treg have multiple specificities and may lead to global immunosuppression (18). In contrast, antigen-specific Treg accumulate in target tissues and local lymphoid compartments where antigen presentation takes place, reducing the risk of off-target immunosuppression and making them both more efficacious and safer than polyclonal Treg for adoptive cell therapy.

Circulating Treg constitute only 1-2% of peripheral blood lymphocytes in humans (19-22) and the frequency of islet antigen-specific Treg in the blood is much lower. Isolating such rare cells is difficult and successfully expanding them to a clinically relevant number has not been reported to date. These challenges have motivated investigators to develop antigen-specific Treg through the transduction of TCRs with known specificities into Treg (8). TCR-transduced Treg selectively localize to the targeted tissue and can exert antigen-specific and bystander suppression (11, 13, 14, 23). However, as a therapeutic application, this approach has limitations due to the overall scarcity of Treg in the blood. Additionally, a fraction of Treg found in the blood are unstable under autoimmune inflammatory conditions (24-27) leading to concerns that extensive expansion may lead to loss of FOXP3 expression and reversion to an effector phenotype (8, 28, 29).

A gene editing approach designed to enforce FOXP3 expression in primary CD4⁺ T cells is disclosed herein (30). Introduction of a strong promoter element, MND, into the endogenous FOXP3 locus by homology directed repair (HDR)-mediated gene editing, mediated stable FOXP3 expression in human CD4⁺ T cells, resulting in robust production of engineered cells with Treg phenotype and suppressive function (EngTregs). As disclosed herein, this novel therapeutic platform was significantly expanded by combining FOXP3 gene editing with human TCR gene transfer to generate antigen-specific EngTregs from primary conventional CD4⁺ T cells. As disclosed herein, the capacity of these antigen-specific cell products to suppress both direct and bystander Teff responses via a variety of mechanisms in vitro and in vivo was demonstrated

### Results

### Generation of Islet-Specific EngTregs by FOXP3 HDR-Editing and LV TCR Transduction

Human islet-specific EngTregs were generated using lentiviral vectors (LV) encoding islet-specific TCRs in conjunction with an approach to induce FOXP3 expression using HDR-based gene editing disclosed herein (30). TABLE 5 shows the six different islet-specific TCRs used in this study, derived from Teff isolated from individuals with T1D.

TABLE 5

| TCR | Islet | Epitope | Epitope | SEQ ID |
|---|---|---|---|---|
| name | antigen | location | sequence | NO: |
| T1D2 | IGRP | 305-324 | QLYHFLQIPT | SEQ ID |
| | | | HEEHLFYVLS | NO: 1430 |
| T1D4 | IGRP | 241-260 | KWCANPDWIH | SEQ ID |
| | | | IDTTPFAGLV | NO: 1431 |
| T1D5-1 | IGRP | 305-324 | QLYHFLQIPT | SEQ ID |
| | | | HEEHLFYVLS | NO: 1432 |
| T1D5-2 | IGRP | 305-324 | QLYHFLQIPT | SEQ ID |
| | | | HEEHLFYVLS | NO: 1433 |
| 4.13 | GAD65 | 553-572 | KVNFFRMVIS | SEQ ID |
| | | | NPAATHQDID | NO: 1434 |
| GAD265 | GAD65 | 265-284 | KGMAALPRLI | SEQ ID |
| | | | AFTSEHSHFS | NO: 1435 |
| PPI76 | Prepro- | 76-90 | SLQPLALEGS | SEQ ID |
| | insulin | | LQKRG | NO: 1436 |

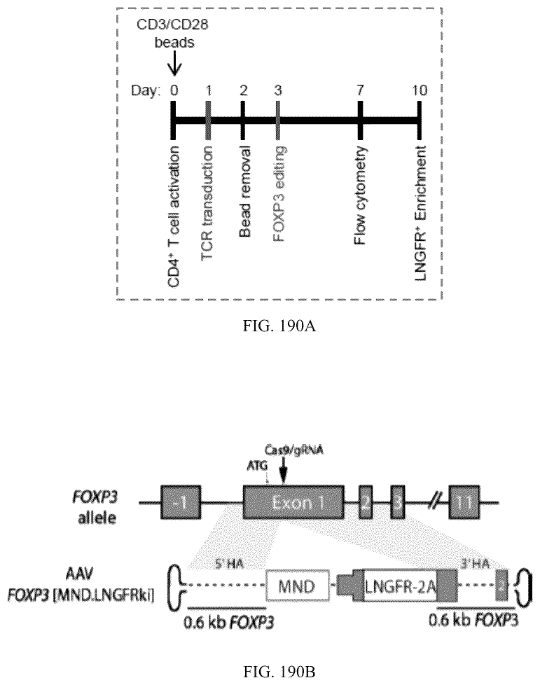

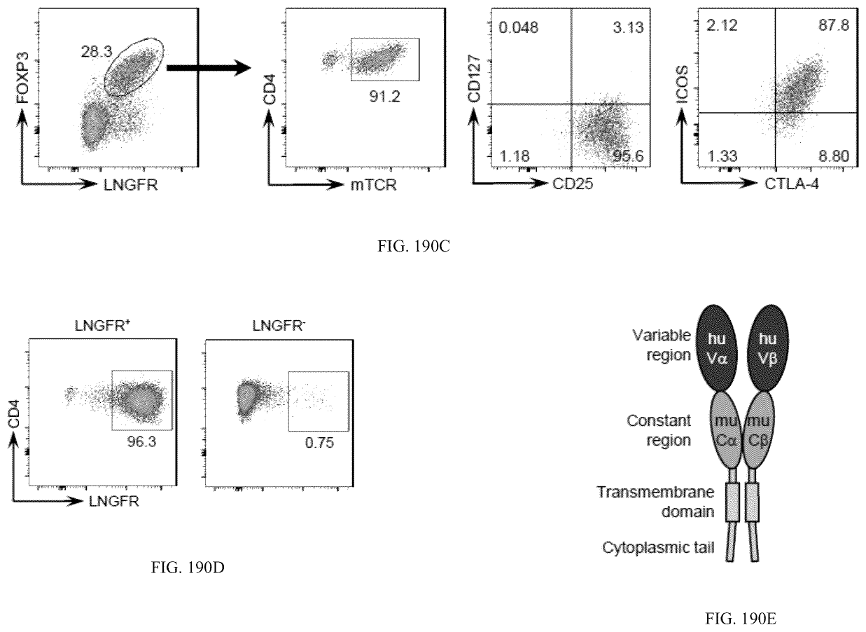

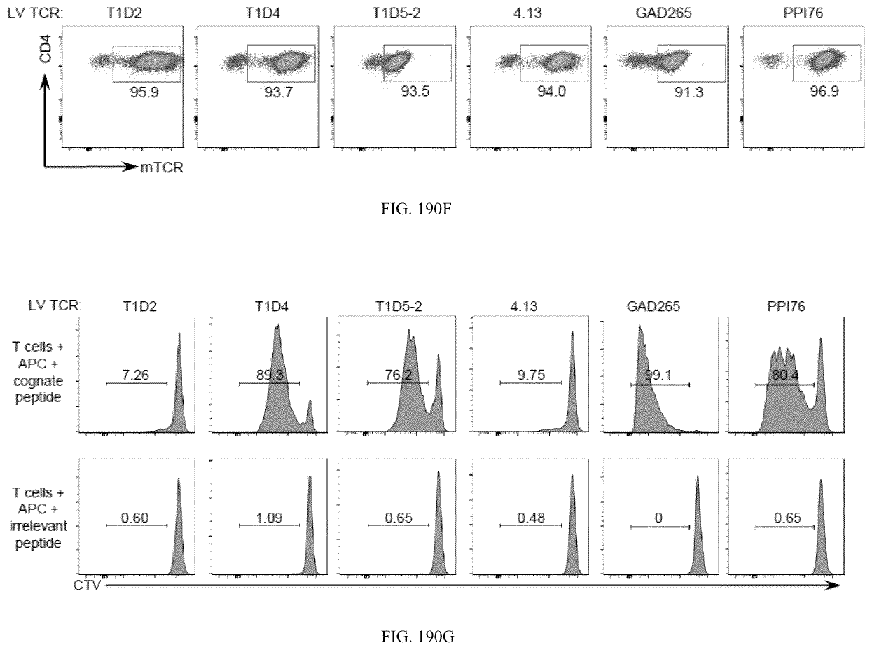

Notably all are HLA-DR0401 restricted and targeted distinct antigens; three recognized islet-specific glucose-6-phosphatase-related protein (IGRP), two recognized glutamic acid decarboxylase (GAD65) and one recognized pre-proinsulin (PPI) (31) and unpublished data). Importantly, these TCR specificities enabled assess to suppression of Teff responses by islet-specific Treg in a number of scenarios including: Treg and Teff having TCRs restricted to the same peptide-MHC complex; Treg and Teff having TCR restricted to different peptides within the same antigen; and Treg and Teff having TCRs with different antigen specificities. For each TCR, an expression cassette for the alpha and beta chain variable regions was cloned into a lentiviral backbone, and included the murine TCR constant region to ensure specificity of pairing between the transgenic TCR chains and permit antibody detection of the exogenous TCR (FIG. 190E, FIG. 190F). Antigen specificity of LV TCR transduced T cells was confirmed using a dye-based proliferation assay with proliferation occurring only in the presence of cognate peptide FIG. 190G). LV encoding islet-specific TCRs were next used to generate islet-specific engineered Treg (islet-specific EngTregs) as outlined in FIG. 190A. In brief, primary human CD4⁺ T cells were transduced with LV encoding islet-specific TCR after 24 h activation with CD3/CD28 beads. Two days after LV transduction, HDR editing of the FOXP3 locus was performed using CRISPR/Cas9 and an AAV6 donor template as described previously (30). As part of this donor cassette, a cis-linked, truncated LNGFR coding sequence (cytoplasmic domain deleted) (32) was introduced within exon 1 and separated by a P2A sequence to enable ribosomal skipping during translation (FIG. 190B). Inclusion of LNGFR allowed tracking and enrichment of the edited cells. Of the resulting transduced and edited T cells, 25-40% co-expressed intracellular FOXP3 and surface LNGFR, 70-95% of which expressed the transduced islet-specific TCR (FIG. 190C). In addition, transduced and edited cells were CD25⁺ CD127⁻ and upregulated CTLA-4 and ICOS expression, consistent with a Treg-like phenotype (30, 33-35). In the following study, these cells are referred to as islet-specific EngTregs.

### Islet-Specific EngTregs Exhibit Antigen-Specific Suppression of Teff Proliferation and Cytokine Production

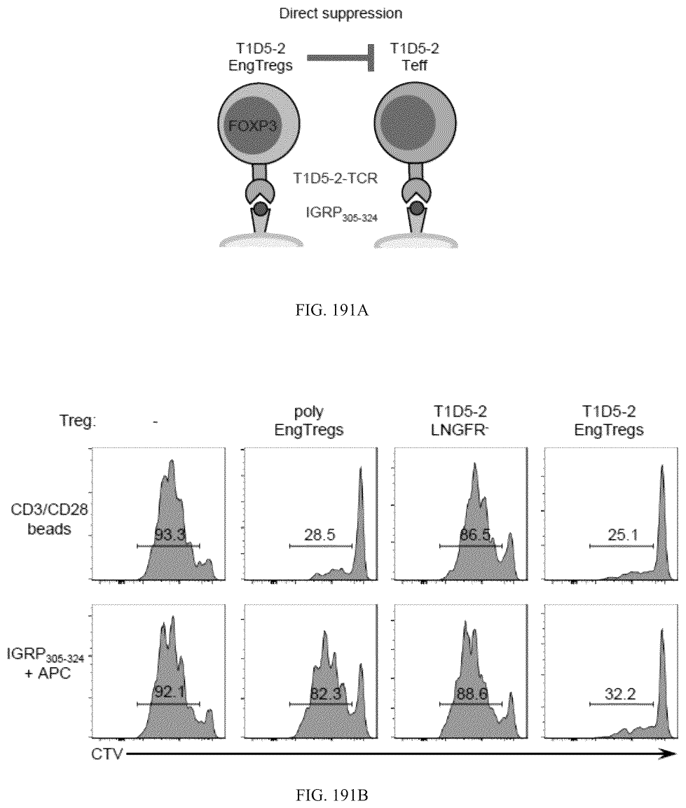

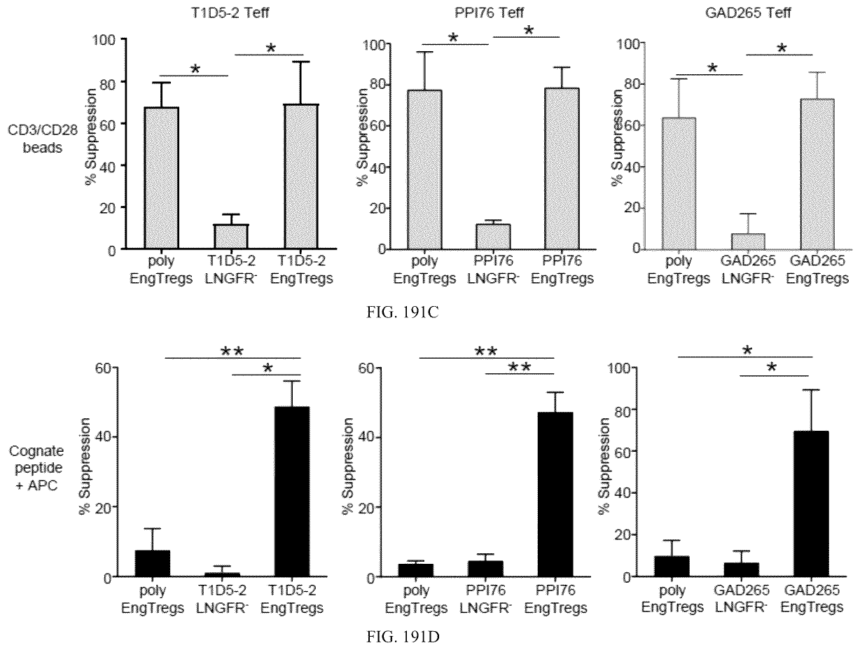

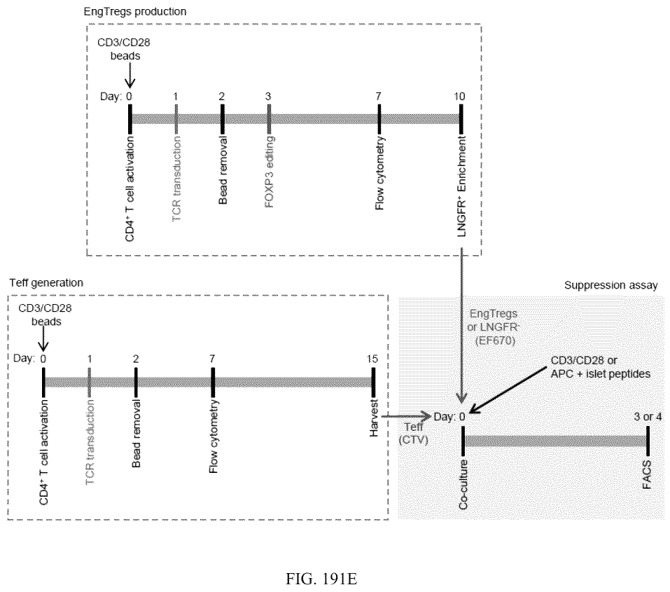

To evaluate the suppressive function of islet-specific EngTregs, their effect was assessed on the proliferation of autologous Teff expressing the same islet-specific TCR in an in vitro suppression assay. Islet-specific EngTregs were enriched using LNGFR antibody affinity beads to greater than 85% purity (FIG. 190D); autologous Teff were prepared by transducing primary human CD4⁺ T cells with LV expressing the same islet TCR (FIG. 191E). Controls were untransduced EngTregs expressing endogenous polyclonal TCRs (henceforth referred to as poly EngTregs), and LV TCR-transduced T cells that were LNGFR⁻ (non-binding fraction during LNGFR affinity bead enrichment; FIG. 190D), henceforth referred to as islet-specific LNGFR⁻ T cells. Islet-specific EngTregs were co-cultured with cell trace violet (CTV)-labeled Teff in the presence of CD3/CD28 beads with CTV dilution used as a measure of T_{eff} proliferation (FIG. 191A, FIG. 191B). Suppressive capacity was tested for the following islet-specific TCRs: T1D5-2 TCR specific for IGRP₃₀₅₋₃₂₄; PPI76 TCR specific for PPI₇₆₋₉₀; and GAD265 TCR specific for GAD65₂₆₅₋₂₈₄. It was confirmed that islet-specific EngTregs were able to suppress CD3/CD28 bead-induced Teff proliferation to similar levels as poly EngTregs (FIG. 191B, FIG. 191C). In contrast, islet-specific LNGFR⁻ T cells had no effect on CD3/CD28 bead-induced Teff proliferation, demonstrating that the suppressive capacity was derived from FOXP3 editing (FIG. 191B, FIG. 191C). It was investigated whether the islet-specific EngTregs suppressed Teff proliferation in an antigen-specific manner by culturing in the presence of cognate peptide and APC. It was found that islet-specific EngTregs significantly suppressed antigen-induced Teff proliferation whereas poly EngTregs and islet-specific LNGFR⁻ T cells did not (FIG. 191B, FIG. 191D). Notably, similar results were observed for all three islet-specific TCRs (FIG. 191B, FIG. 191C, FIG. 191D).

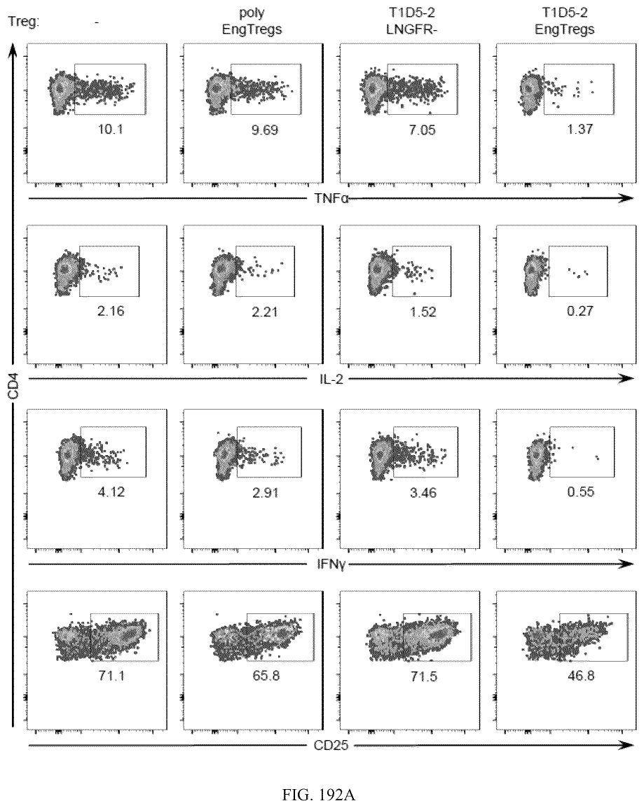

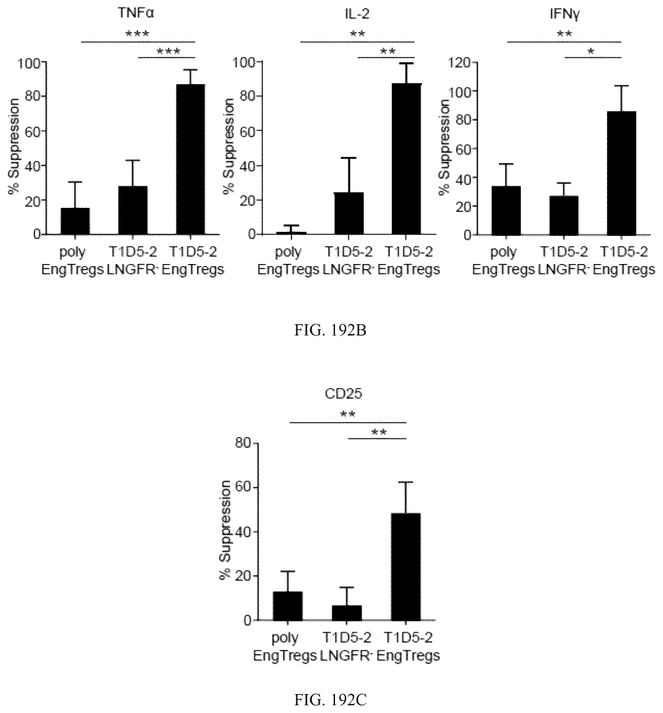

Since Treg have been reported to also suppress cytokine production by Teff (36-39), it was examined whether islet-specific EngTregs also suppress Teff cytokine production. For this experiment, both EngTregs and Teff expressed the T1D5-2 TCR and were cocultured in the presence of cognate IGRP₃₀₅₋₃₂₄ peptide and APC. Teff production of TNFα, IL-2 and IFNγ was determined by intracellular cytokine staining. Islet-specific EngTregs significantly suppressed antigen-induced Teff production of TNFα, TL-2 and IFNγ compared to poly EngTregs or islet-specific LNGFR⁻ T cells, both of which had no significant effect (FIG. 192A, FIG. 192B). In addition, islet-specific EngTregs also suppressed Teff expression of the early activation marker CD25 (FIG. 192A, FIG. 192C). Collectively, these results indicated that antigen-specific suppression required not only suppressive capacity derived from FOXP3 editing, but also specific TCRs that received antigen-stimulation. They also demonstrated that islet-specific EngTregs exhibited antigen-specific suppressive capacity with respect to both Teff proliferation and cytokine production.

### Islet-Specific EngTregs Manifest Antigen-Specific Bystander Suppression

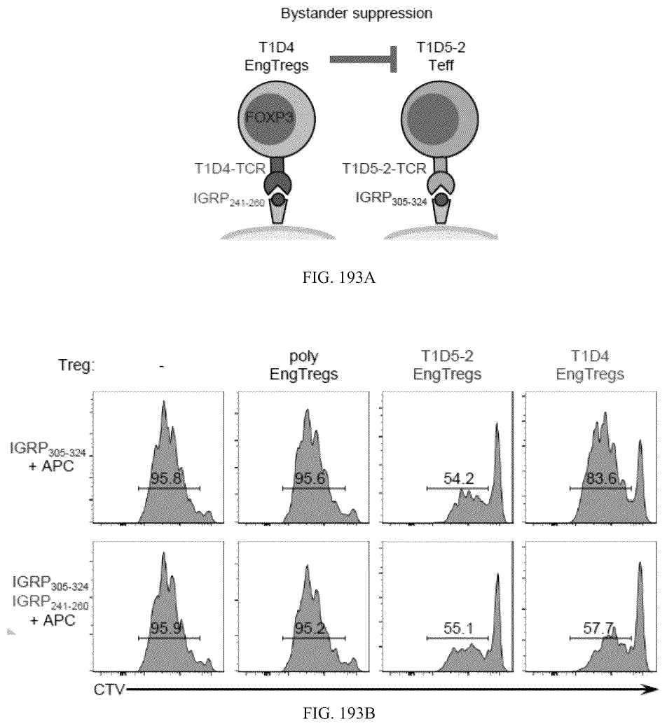

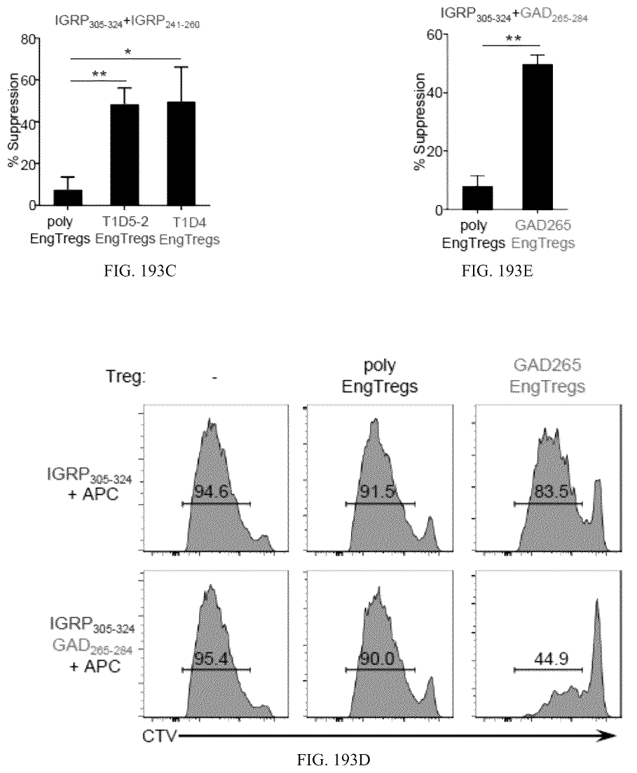

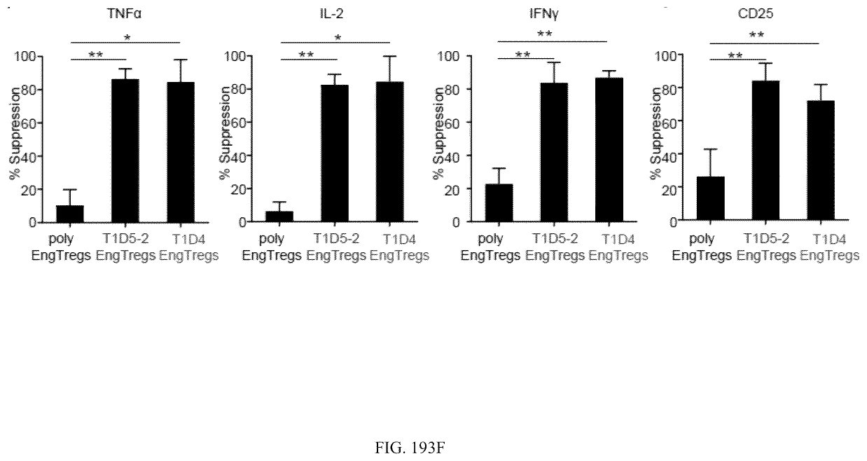

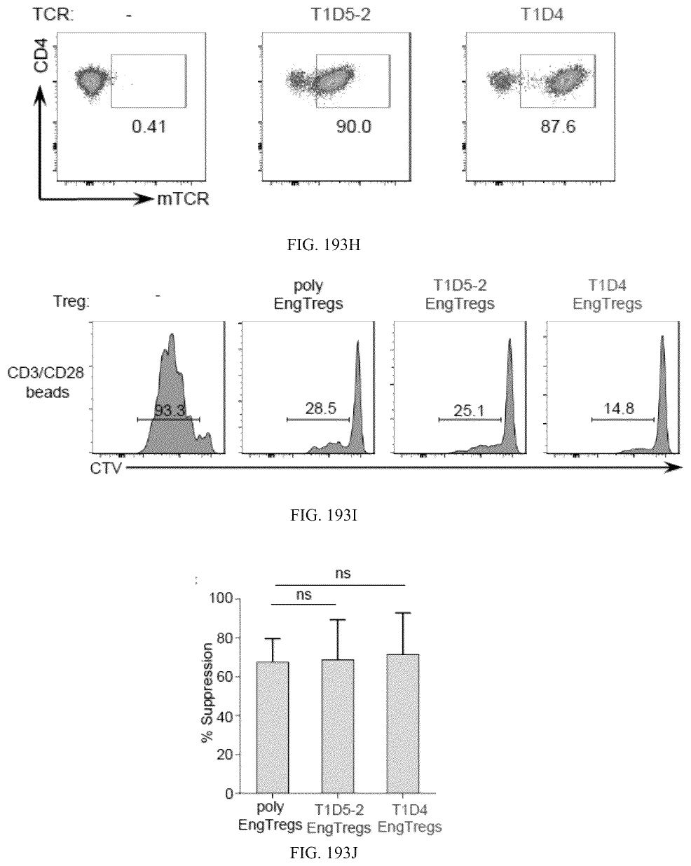

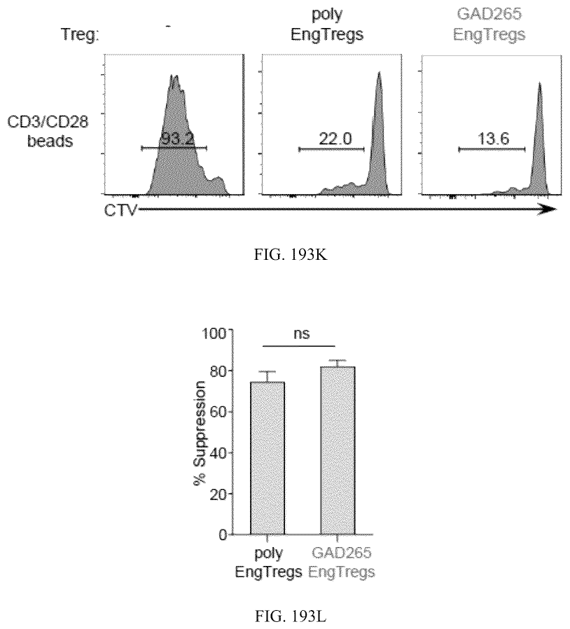

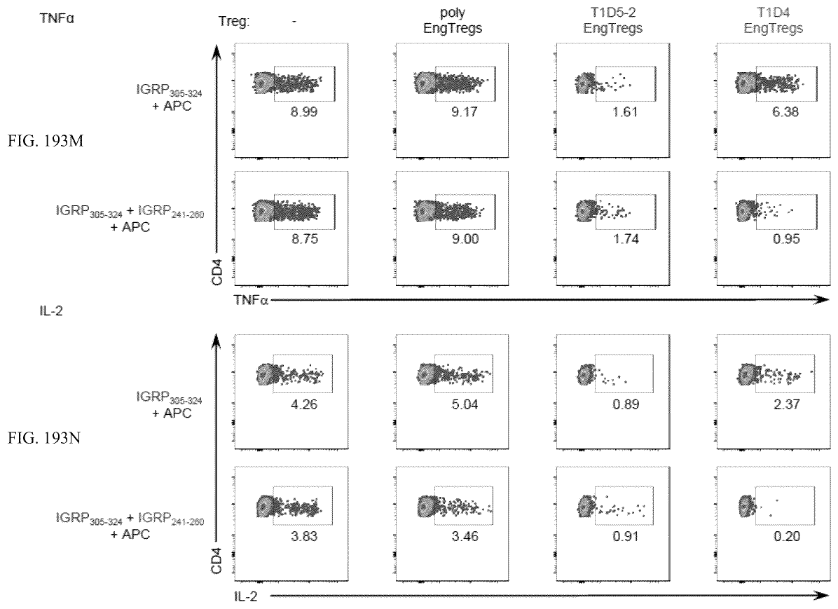

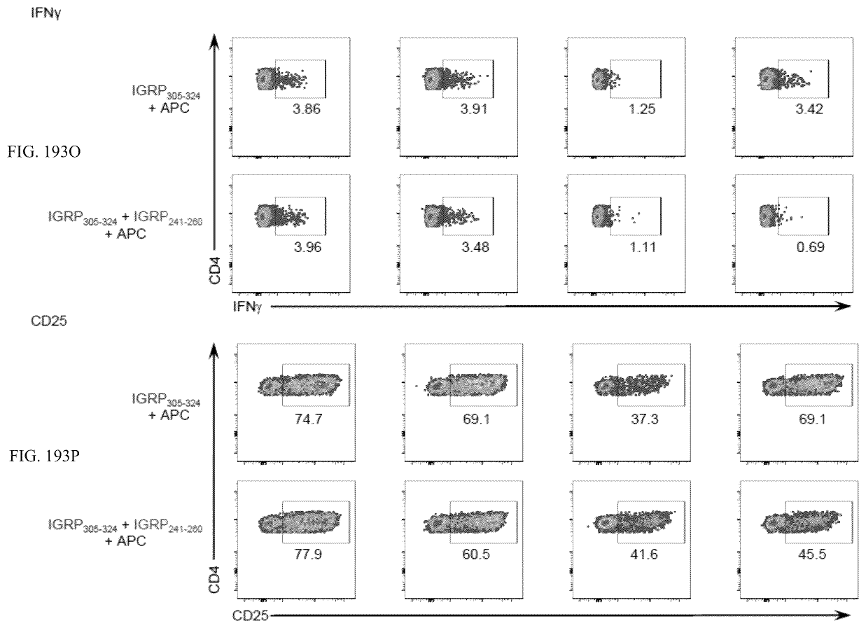

Activation of Treg is antigen-specific. However, once activated, Treg have the ability to exert bystander suppression (8, 40). This characteristic is especially important in the context of treating autoimmunity, where autoreactivity targets multiple tissue antigens. To determine whether islet-specific EngTregs can exert bystander suppression, it was investigated whether islet-specific EngTregs expressing the T1D4 TCR were able to suppress Teff expressing the T1D5-2 TCR (FIG. 193A). Note that T1D4 and T1D5-2 recognized two different IGRP epitopes, IGRP₂₄₁₋₂₆₀ and IGRP₃₀₅₋₃₂₄, respectively. T1D4 islet-specific EngTregs were co-cultured with T1D5-2 Teff in the presence of APC pulsed with either the T1D5-2 cognate peptide (IGRP₃₀₅₋₃₂₄) alone, or with a mixture of IGRP₃₀₅₋₃₂₄ plus the T1D4 cognate peptide (IGRP₂₄₁₋₂₆₀). Control Treg included poly EngTregs and T1D5-2 islet-specific EngTregs. Importantly, TCR expression levels were equivalent for both T1D4 and T1D5-2 in edited cells (FIG. 193H) and all EngTregs, irrespective of TCR, exerted similar Teff suppression in response to CD3/CD28 bead stimulation (FIG. 193I, FIG. 193J). As expected, and consistent with FIG. 191A-FIG. 191D, T1D5-2 Teff proliferation was suppressed by the T1D5-2 islet-specific EngTregs in the presence of either the cognate peptide IGRP₃₀₅₋₃₂₄ alone or with both peptides (FIG. 193B, FIG. 193C). In contrast, T1D5-2 Teff proliferation was only suppressed by T1D4 islet-specific EngTregs when both IGRP₂₄₁₋₂₆₀ and IGRP₃₀₅₋₃₂₄ peptides were present (FIG. 193B, FIG. 193C), findings consistent with bystander suppression. In contrast, islet-specific LNGFR⁻ T cells showed neither direct nor bystander suppression of Teff proliferation, although they were activated by their cognate peptides (data not shown). Importantly, the capacity for bystander suppression was not limited to EngTregs with IGRP-specific TCRs. Bystander suppression was also detected for EngTregs expressing the GAD265 TCR, which suppressed proliferation of T1D5-2 Teff when both GAD₂₆₅₋₂₈₄ and IGRP₃₀₅₋₃₂₄ peptides were present (FIG. 193D, FIG. 193E). Bystander suppression was not observed using poly EngTregs, although they did show comparable suppression as GAD265 islet-specific EngTregs on T1D5-2 Teff proliferation induced by CD3/CD28 beads (FIG. 193K, FIG. 193L). In parallel studies, bystander suppression was tested in the context of Teff cytokine production, again utilizing T1D4 islet-specific EngTregs and T1D5-2 Teff. Similar evidence of bystander suppression was observed fro: IGRP₃₀₅₋₃₂₄-specific cytokine production and CD25 expression by T1D5-2 Teff were inhibited by T1D4 islet-specific EngTregs only when its cognate peptide IGRP₂₄₁₋₂₆₀ was present in addition to IGRP₃₀₅₋₃₂₄ (FIGS. 193F-193Q). In contrast, cytokine production and CD25 expression by T1D5-2 Teff was suppressed by T1D5-2 EngTregs in the presence of IGRP₃₀₅₋₃₂₄ alone or in combination with IGRP₂₄₁₋₂₆₀ (FIGS. 193F-193Q). In summary, these combined findings showed that islet-specific EngTregs had the ability to provide bystander suppression that limited both Teff proliferation and cytokine production.

### Islet Specific EngTregs Suppress Polyclonal Islet-Specific T Cells from T1D Subjects Across Multiple Specificities

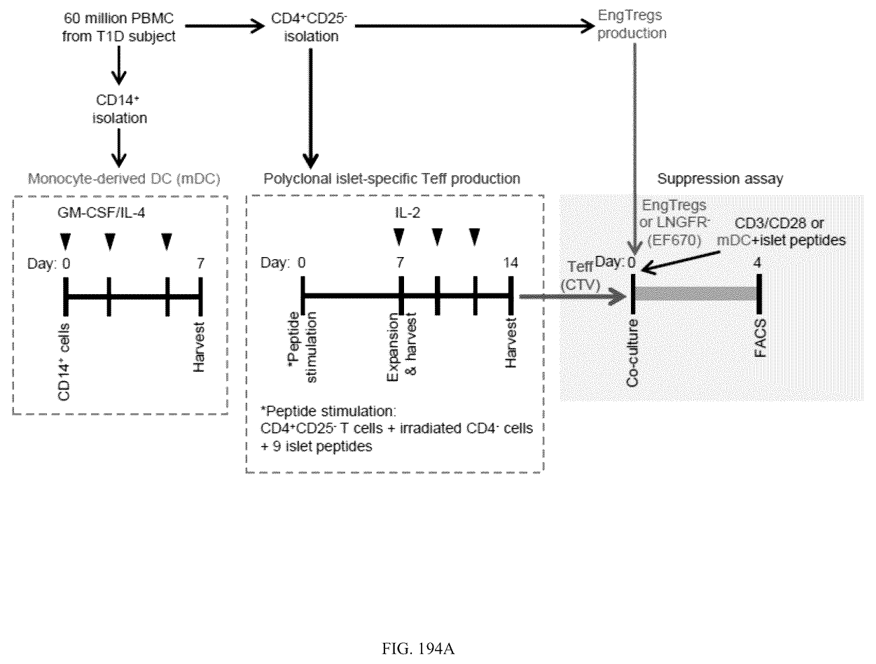

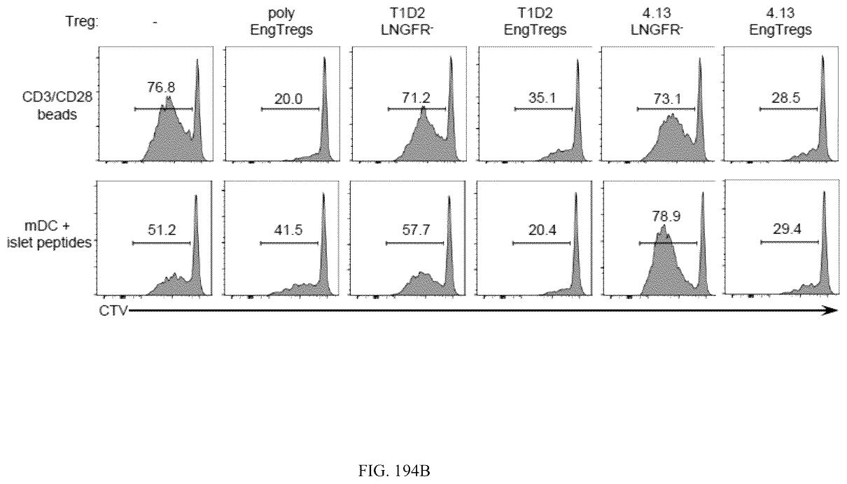

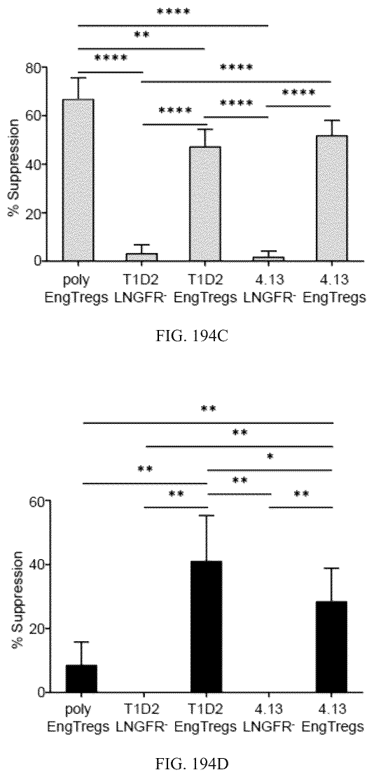

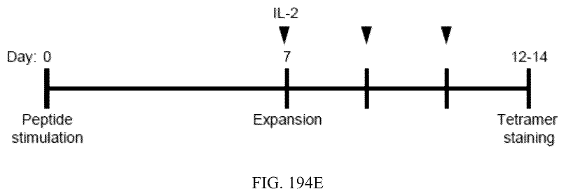

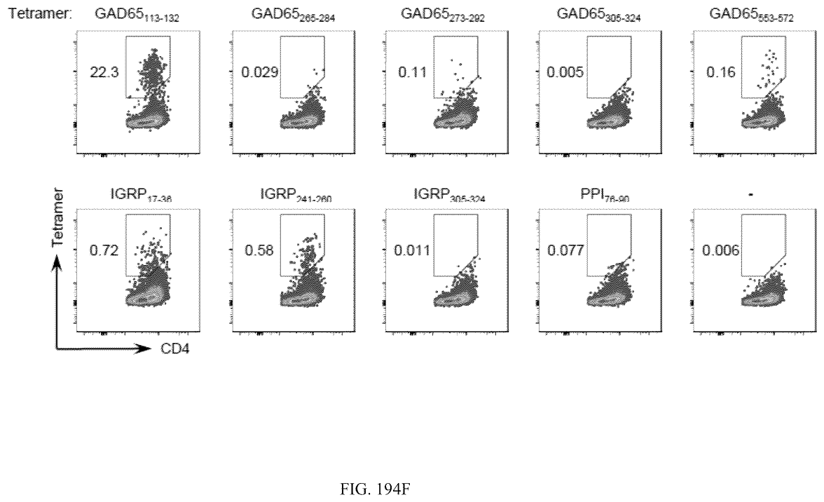

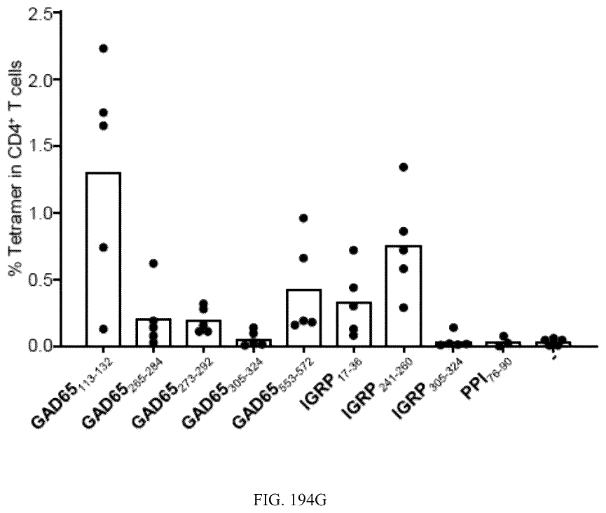

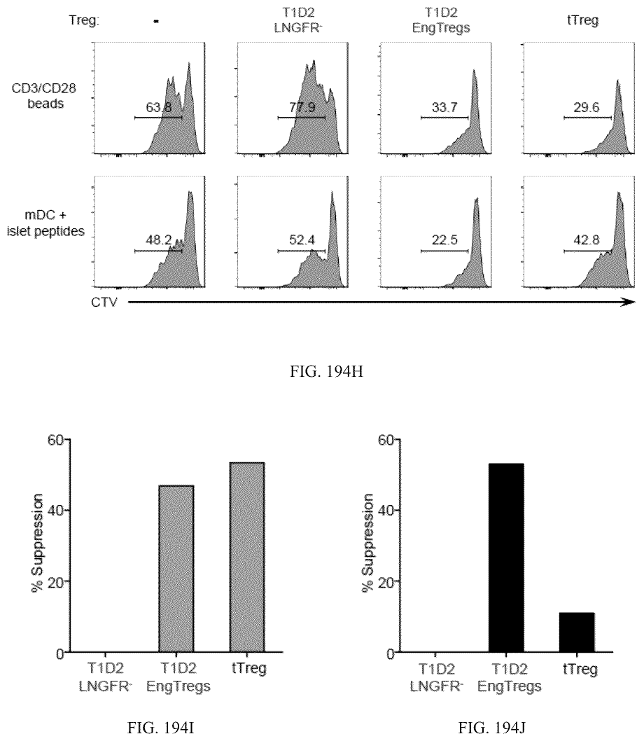

An initial assessment of the ability of islet-specific EngTregs to suppress in an antigen specific manner utilized Teff that were themselves transduced with LV encoding TCRs. However, the ultimate therapeutic goal is to suppress polyclonal islet-specific T cells in individuals at risk or with T1D. Therefore, a strategy was designed to assess the activity of islet-specific EngTregs against endogenous islet-specific Teff derived from PBMC of T1D subjects. Using PBMC from T1D donors, a parallel approach was used to generate: a) monocyte-derived DC (mDC) for use as APC; b) polyclonal islet-specific Teff; and c) EngTregs (FIG. 194A). To obtain islet specific Teff, CD4⁺CD25⁻ cells were cultured with irradiated autologous APC and a pool of 9 islet-specific peptides for 12-14 days (FIG. 194A, FIGS. 194E-194G). Peptides were chosen that were derived from IGRP, GAD65, and PPI that were known to be presented on HLA-DR0401 and for which HLA Class II tetramers were available (31, 41-45). This approach enabled Teff enriched for a mixture of islet specificities to be obtained, determined by tetramer staining, from multiple individuals with T1D. A broad range of tetramer positive cell frequencies was observed across donors, and T cells specific to GAD₁₁₃₋₁₃₂ and IGRP₂₄₁₋₂₆₀ were detected at a greater frequency than other specificities (FIG. 194F, FIG. 194G).

In parallel, CD4⁺ T cells from the same T1D donors were used to generate autologous T1D2 islet-specific EngTregs and 4.13 islet-specific EngTregs, with TCRs restricted to IGRP₃₀₅₋₃₂₄ and GAD65₅₅₃₋₅₇₃, respectively. These peptides were present among the islet peptide pool used to stimulate the polyclonal Teff (FIGS. 194E-194G). In a control experiment to test antigen-independent suppressive capacity, autologous poly EngTregs, T1D2 islet-specific EngTregs, and 4.13 islet-specific EngTregs exhibited comparable suppression of CD3/CD28 triggered Teff proliferation (FIG. 194B, FIG. 194C). In the setting of antigen-stimulation, polyclonal islet Teff proliferated in the presence of mDC and a mixture of 9 islet peptides (FIG. 194B, FIG. 194D). Poly EngTregs and islet-specific LNGFR⁻ T cells regardless of their TCR did not mediate suppression of polyclonal islet enriched Teff. Strikingly, proliferation of islet peptide-specific T1D Teff was specifically suppressed by both T1D2 islet-specific EngTregs and 4.13 islet-specific EngTregs (FIG. 194B, FIG. 194D). Superior suppressive capacity was confirmed for islet-specific EngTregs under islet-specific stimulation and that expanded natural/thymic Treg (tTreg) did not exert notable Teff suppression (FIGS. 194H-194J). Together, these findings directly demonstrated that islet-specific EngTregs generated from individuals with T1D exhibit the capacity to mediate both antigen-specific and bystander suppression of autologous, autoreactive, islet-specific Teff.

### EngTregs Utilize Both Contact-Dependent and -Independent Suppressive Mechanisms

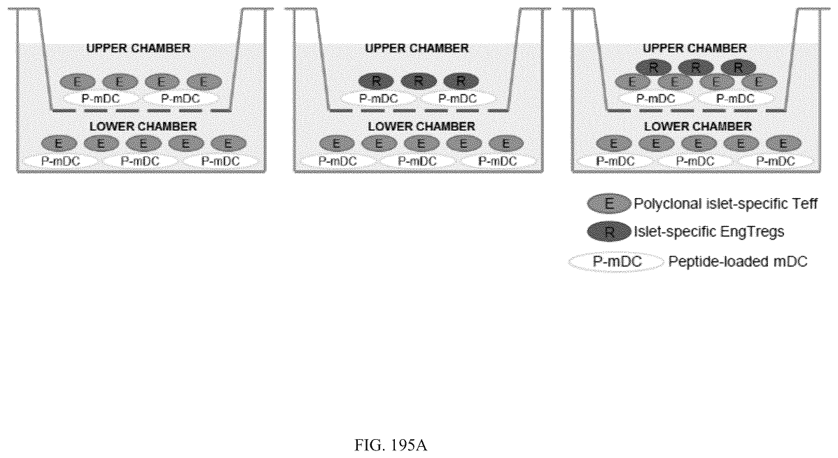

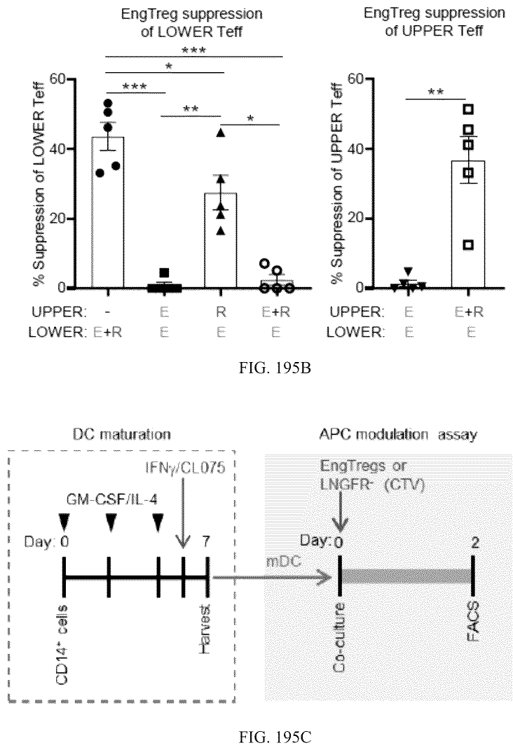

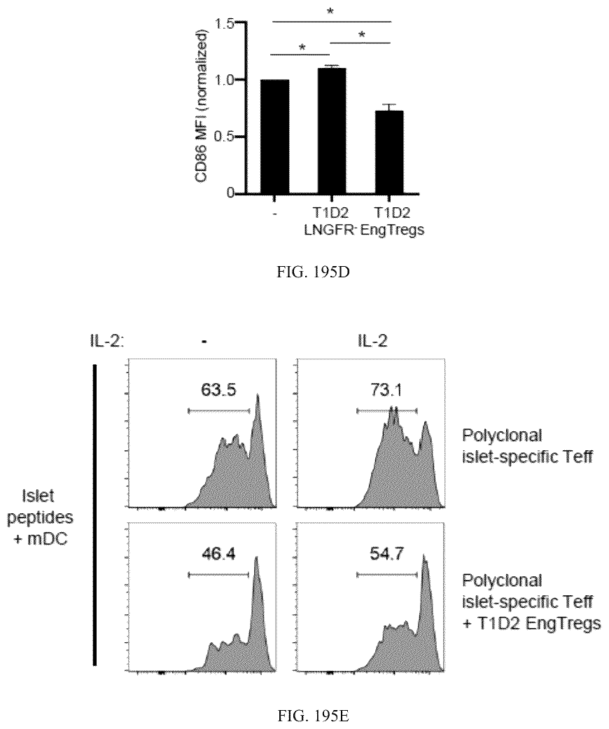

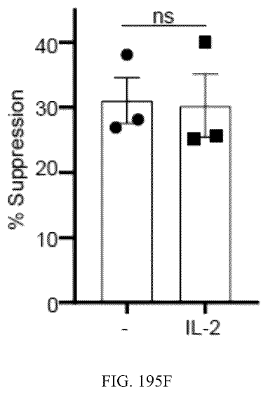

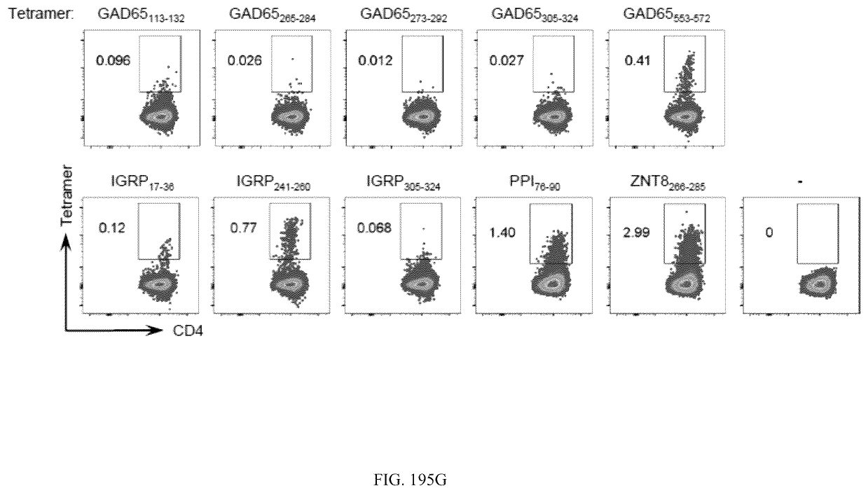

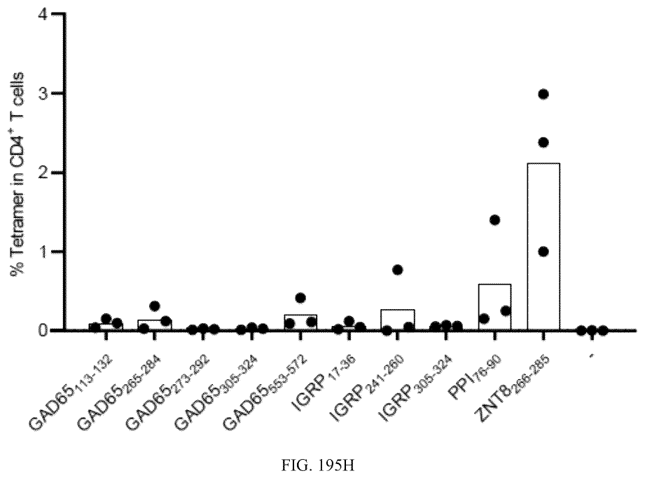

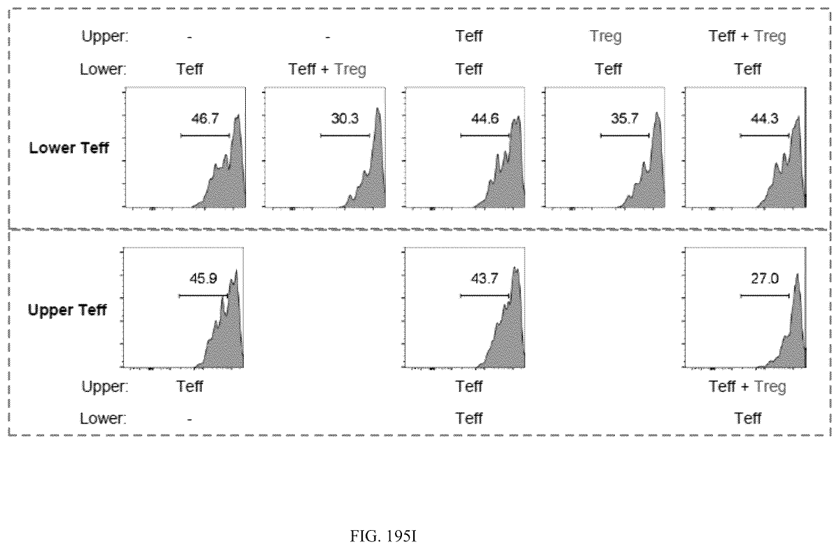

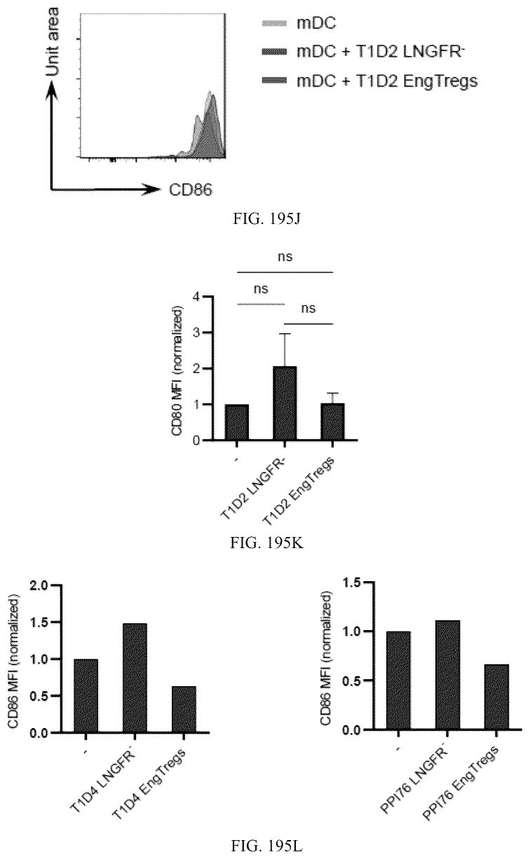

Tregs mediate suppression via multiple mechanisms including expression of anti-inflammatory soluble mediators, inhibition of APC maturation and consumption of IL-2 (8, 46). These mechanisms may also used by human, islet-specific, EngTregs. To investigate contact-dependent and -independent mechanisms, a transwell-based assay was used to assess the role for soluble factors produced by EngTress (FIG. 195A) (47, 48). Polyclonal islet-specific Teff were generated from CD4⁺CD25⁻ T cells from T1D subjects as above and in FIGS. 195G-194I. In the upper transwell chamber, T1D2 islet-specific EngTregs were plated either alone or co-cultured with polyclonal islet-specific Teff, and in the lower chamber, polyclonal islet-specific Teff were plated. Peptide loaded mDC were plated in both chambers and cell numbers were kept equivalent between chambers (FIG. 195A). T1D2 islet-specific EngTregs plated without Teff in the upper chamber significantly suppressed the proliferation of polyclonal islet-specific Teff in the lower chamber (FIG. 195B left, FIG. 195I). Thus, islet-specific EngTregs can mediate contact-independent suppression, presumably via production of transwell permeable soluble factors. However, contact-independent suppression was incomplete and was lower than that the positive control where the islet-specific EngTregs and the polyclonal islet-specific Teff were in direct contact (FIG. 195B left, FIG. 195I). Further, cell proximity also impacted the experimental outcome. EngTreg preferentially suppressed Teff that in closest proximity. T1D2 islet-specific EngTreg in the upper chamber suppressed proliferation of upper chamber Teff but had no effect on lower chamber Teff when Teff were present in both chambers (FIG. 195B left, FIG. 195I).

To determine whether islet-specific EngTregs could inhibit APC maturation, the effect of T1D2 islet-specific EngTregs on APC expression of CD80 and CD86 was assessed. In this assay, autologous monocytes restricted to HLA-DR0401 were matured into DC and then co-cultured with T1D2 islet-specific EngTregs in the presence of its cognate peptide IGRP₃₀₅₋₃₂₄ for 2 days (FIG. 195C). T1D2 islet-specific EngTregs were able to suppress mDC activation as measured by reduced mDC expression of CD86 compared to DCs alone or T1D2 islet-specific LNGFR⁻ T cells (FIG. 195D; FIG. 195J). However, in contrast to previous studies showing that Tregs can also inhibit APC expression of CD80 (49, 50), islet-specific EngTregs had no impact on CD80 expression (FIG. 195K). Similar results were observed for T1D4 islet-specific EngTregs and PPI76 islet-specific EngTregs with both demonstrating ability to suppress CD86 expression on mDC but having no effect on CD80 expression (FIG. S8, and C).

The potential contribution of IL-2 consumption on EngTreg-mediated suppression was investigated. In mice, Treg consumption of IL-2 leads to cytokine deprivation-mediated apoptosis of Teff (51). However, it remains unclear whether this mechanism is operative in human Tregs with several studies reporting that IL-2 depletion is not required for Treg suppressive capacity (46, 52). Here, whether EngTreg suppression could be reversed by excess IL-2 was investigated, and found that addition of exogenous IL-2 had no significant effect on suppression of polyclonal islet-specific Teff proliferation (FIG. 195E, FIG. 195F).

### Islet-Specific EngTregs with Lower Functional Avidity Exhibit Superior Suppressive Activity

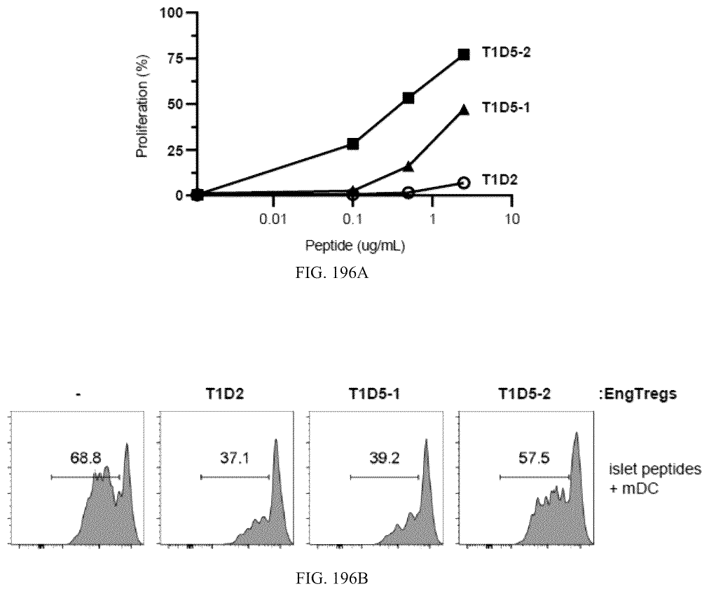

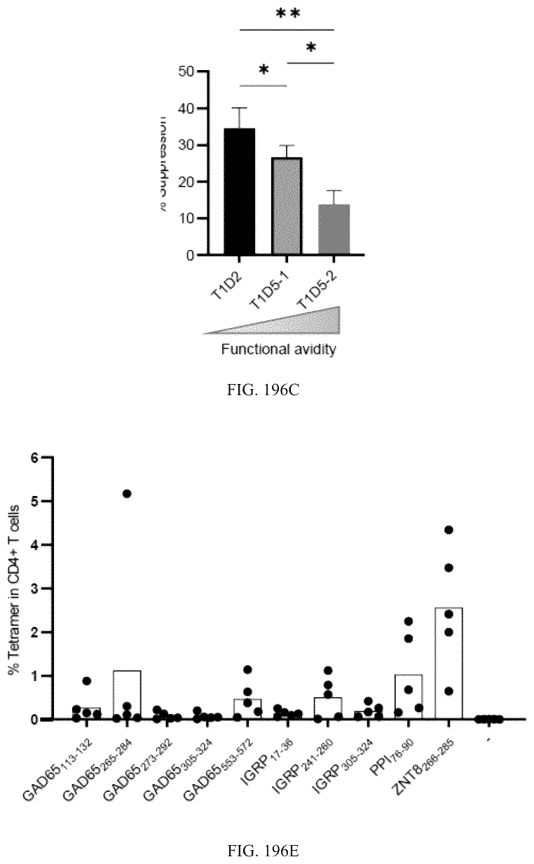

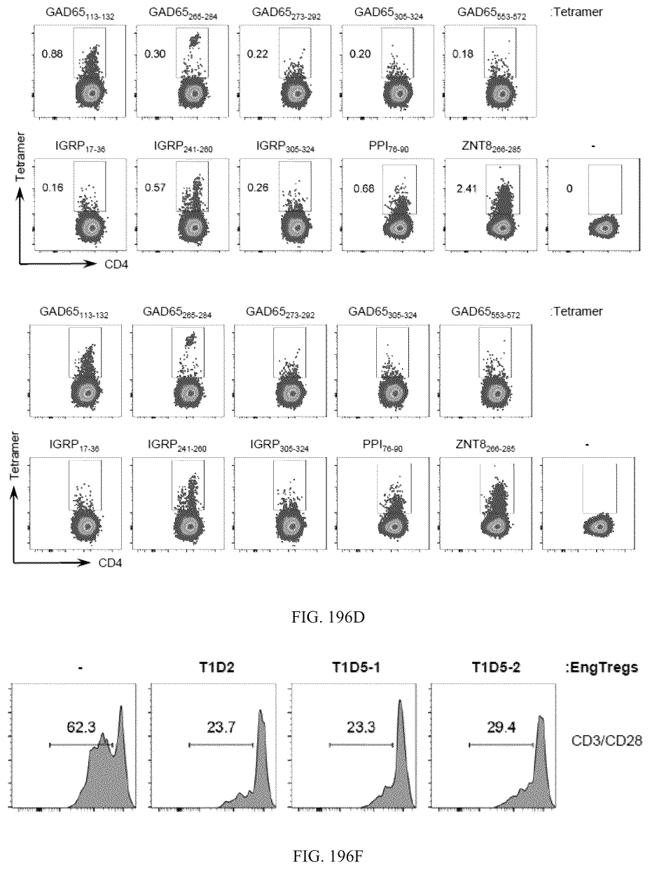

As part of the studies, it was observed that alternative IGRP-specific TCRs utilized in the studies exhibited different functional avidities. Therefore, these unique features were used as way to begin to explore the impact of TCR affinity on islet-specific EngTregs function. T1D2, T1D5-1 and T1D5-2 were compared, each of which recognize the same cognate peptide, IGRP₃₀₅₋₃₂₄, in the context of HLA DR0401 (TABLE 5) (31). As shown in FIG. 196A, these TCRs exhibited different functional avidities in response to cognate peptide as determined in a dose response experiment measuring cell proliferation: T1D5-2 had the highest functional avidity with proliferation at peptide concentrations as low as 0.1 g/ml; followed by T1D5-1 at 1.0 g/ml; and T1D2, with the lowest functional avidity, with proliferation only at 5 g/ml). Based on this information, a side-by-side comparison of the relative suppressive capacity of EngTregs expressing each of these islet-specific TCRs was performed. Proliferation of polyclonal islet-specific Teff (FIG. 196D, FIG. 196E) was measured in the presence of islet-specific peptides and mDC and either T1D2, T1D5-1, or T1D5-2 EngTregs. The data were normalized by suppressive activity obtained from suppression assay set up in parallel using CD3/CD8 beads (FIG. 196F, FIG. 196G). This latter assay provided a baseline control for EngTreg function, as this activation method is not impacted by TCR avidity. Strikingly, T1D2 islet-specific EngTregs, which had the lowest functional avidity, consistently showed the highest percent suppression, followed by T1D5-1 and then T1D5-2 islet-specific EngTregs (FIG. 196B, FIG. 196C; FIG. 196D, FIG. 196E). Together, these data suggested that there was an inverse relationship between TCR functional avidity and antigen-specific Treg suppressive capacity.

### Generation and In Vitro Characterization of Murine Islet-Specific EngTregs

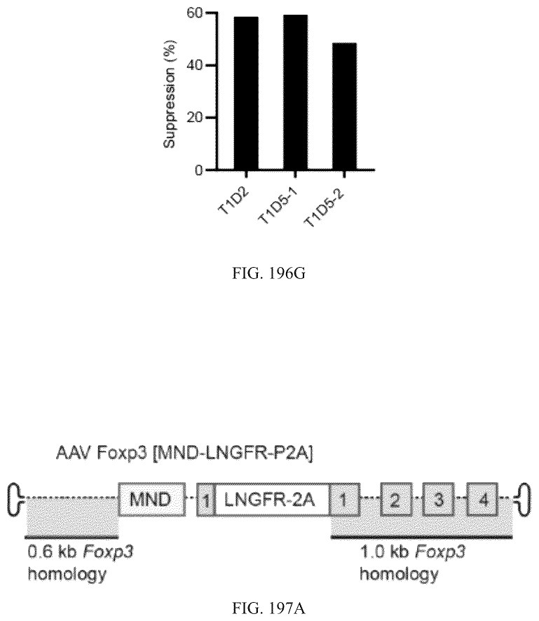

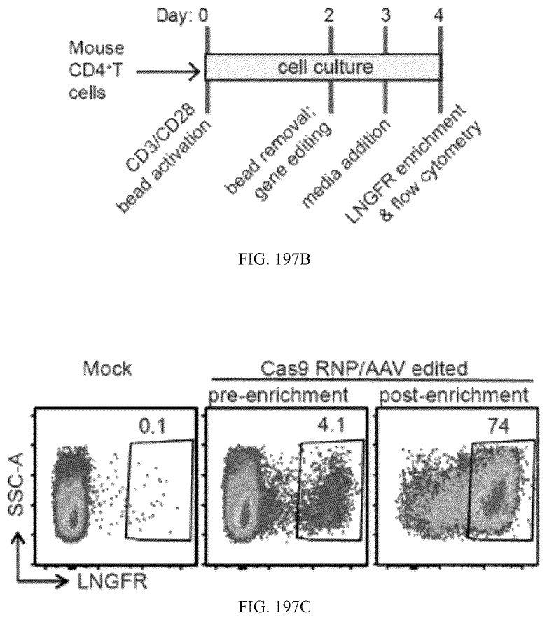

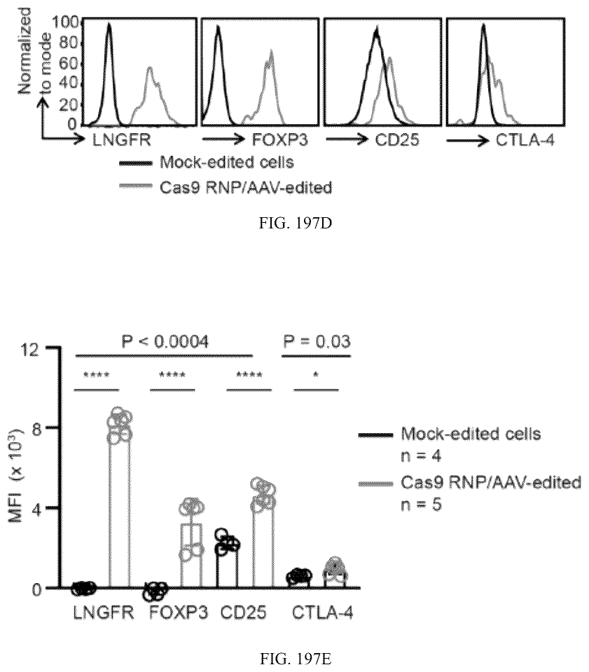

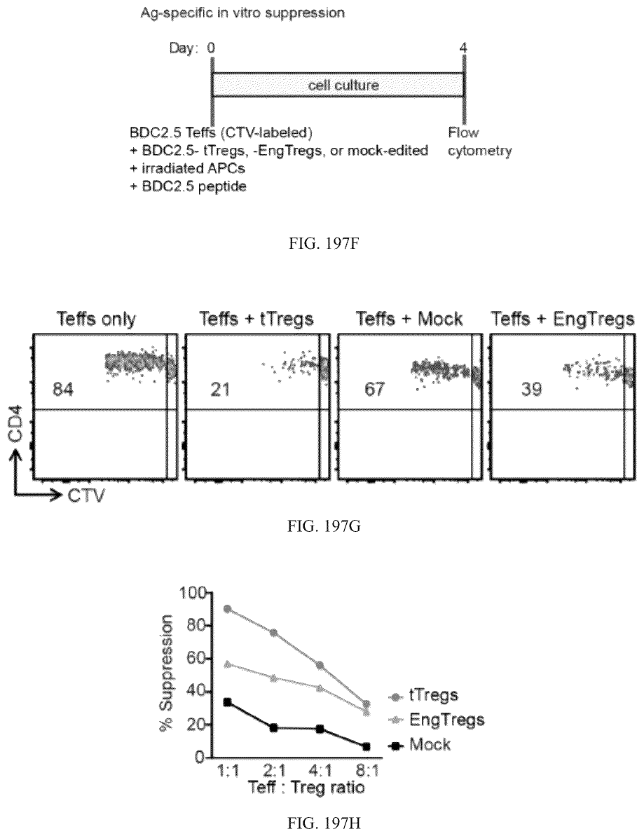

To evaluate the in vivo efficacy of islet-specific EngTregs, methods were established to generate murine islet-specific EngTregs and tested their in vitro functional activity. Similar to the method for generating human islet-specific EngTregs, we used a CRISPR-Cas9-based HDR gene-editing strategy to introduce the MND promoter into the first coding exon of Foxp3, and a truncated LNGFR coding sequence was introduced upstream of Foxp3 (FIG. 197A, FIG. 197B). NOD.Cg-Tg(TcraBDC2.5,TcrbBDC2.5)1Doi/DoiJ (NOD BDC2.5) transgenic mice were used as the source of CD4⁺ T cells as these mice express an islet-specific TCR and rapidly induce diabetes when transferred into non-diabetic NOD mice (53-56). For negative controls, mock-edited NOD BDC2.5 CD4⁺ T cells were used that were electroporated without RNP and cultured in media containing the AAV5 donor template. In contrast to mock-edited cells, NOD BDC2.5 CD4⁺ T cells treated using both RNP and AAV demonstrated sustained LNGFR expression. Column-based LNGFR affinity purification resulted in ˜75% LNGFR⁺ cells (FIG. 197C), referred to hereafter as BDC2.5 islet-specific EngTregs. Enriched BDC2.5 islet-specific EngTregs demonstrated increased expression of LNGFR, FOXP3 and CTLA-4, with similar or higher CD25 expression compared to mock-edited cells (FIG. 197D, FIG. 197E).

The ability of the BDC2.5 islet-specific EngTregs to suppress the proliferation of activated islet-specific NOD BDC2.5 CD4⁺ Teff cells (abbreviated here as islet-specific Teff) in an antigen-dependent manner in vitro was tested. As in the human studies, proliferation by CTV dilution was assessed, and compared the suppressive capacity of BDC2.5-EngTregs, BDC2.5-tTreg and mock-edited cells (FIG. 197F). Both BDC2.5-tTreg and BDC2.5 islet-specific EngTregs showed dose-dependent suppression of BDC2.5-CD4⁺ Teff proliferation in comparison to mock-edited cells (FIG. 197G, FIG. 197H). tTreg displayed slightly better in vitro suppressive function than EngTregs, possibly reflecting the impact of thymic tTreg selection and/or programming in comparison to Teff converted EngTregs.

### Islet-Specific EngTregs Traffic to the Pancreas, Prevent Diabetes, and Stably Persist In Vivo

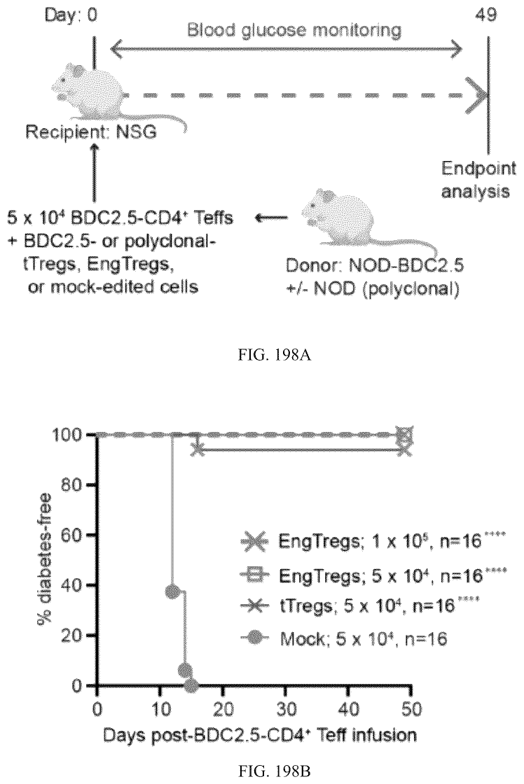

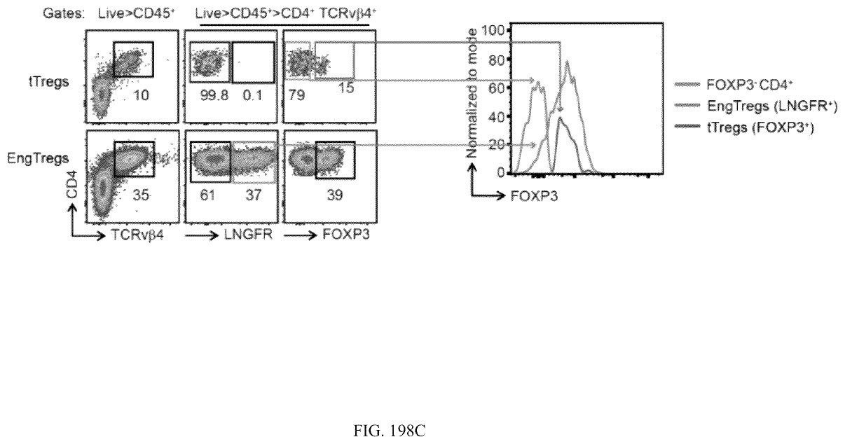

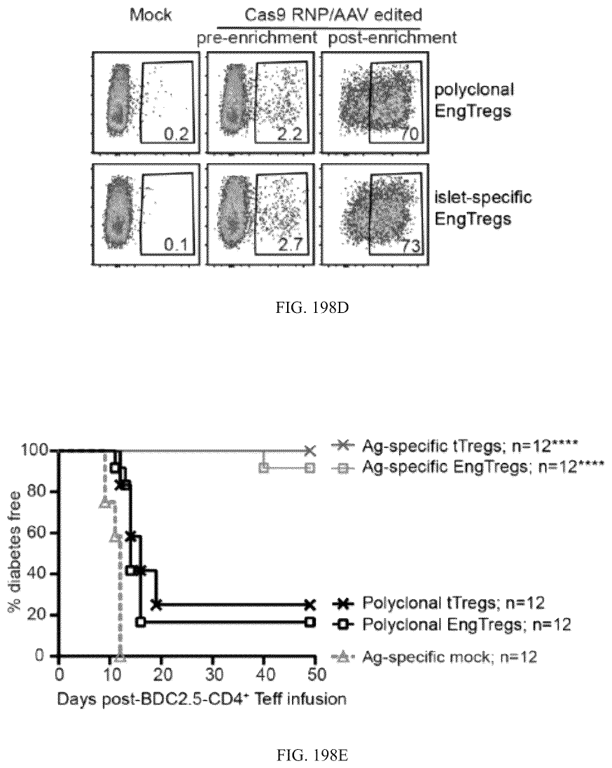

Whether BDC2.5 islet-specific EngTregs could prevent diabetes in vivo using a BDC2.5-CD4+ Teff induced T1D model was determined. In this model, adoptive transfer of BDC2.5-CD4⁺ Teff into immunodeficient nonobese diabetic (NOD)-scid-IL2rγ^{NULL}(NSG) mice rapidly promotes diabetes development as measured by blood glucose analysis (57). One of two doses (5×10⁴ or 1×10⁵) of BDC2.5 islet-specific EngTregs, or 5×10⁴ BDC2.5-tTreg (CD4⁺CD25^{hi} cells, column enriched and activated to match EngTregs) or mock-edited control cells were mixed with 5×10⁴ BDC2.5-CD4⁺ Teff (1:1 or 1:2 Teff:Treg ratios) and injected into 8-10 week old male recipient NSG mice (FIG. 198A). After cell transfer, blood glucose levels were monitored for up to 49 days; mice were sacrificed if they developed diabetes (blood glucose ≥250 mg/dL for two consecutive days). All diabetes-free animals were euthanized on day 49 for tissue and cell analysis. Mice infused with either BDC2.5 islet-specific EngTregs or -tTreg were almost completely diabetes-free, whereas all mice receiving mock-edited control cells developed diabetes within 9-15 days post-Teff transfer (FIG. 198B). Both doses of islet specific EngTregs prevented diabetes development. Thus, BDC2.5 islet-specific EngTregs were as effective as BDC2.5-tTreg in suppressing diabetes onset in this T1D mouse model.

In order to be beneficial, therapeutic Treg must home to the target tissue(s) and persist, maintaining a stable phenotype. To determine whether the islet specific EngTregs homed and persisted in the pancreas, pancreatic lymphocytes were isolated on day 49 by enzymatic digestion and performed flow cytometry to detect donor BDC2.5-CD4⁺ T cells (TCRvβ4⁺) and assessed the expression of LNGFR and FOXP3 (FIG. 198C). TCRvβ4⁺ EngTregs and tTreg were both present in the pancreas of diabetes-free mice on day 49. LNGFR⁺ cells were detected only in animals that received EngTregs (FIG. 198C) and these islet-specific, LNGFR⁺ EngTregs (CD4⁺ TCRvβ4⁺LNGFR⁺) maintained high-levels of FOXP3 expression. Specifically, LNGFR⁺ EngTregs expressed similar levels of intracellular FOXP3 as tTreg (CD4+ TCRvβ4⁺FOXP3⁺ cells); CD4⁺TCRvβ4⁺FOXP3⁻ cells (representing residual Teff cells) within the Treg recipient cohort (FIG. 198C). Together, these data evidence that, like BDC2.5-tTreg, BDC2.5 islet-specific EngTregs home to the pancreas and maintain FOXP3⁺ expression despite the sustained presence of islet-specific Teff.

Multiple reports have shown that islet-specific tTreg but not polyclonal tTreg are effective in preventing diabetes in T1D mouse models (9, 10, 17). To ask whether this was also true for our EngTregs, polyclonal EngTregs and tTreg were generated from NOD mice using identical methods (FIG. 198D) and compared them directly with BDC2.5 islet-specific-EngTregs and -tTreg in the NOD T1D model. 1×10⁵ polyclonal-EngTregs, or -tTreg, or BDC2.5-EngTregs or -tTreg, or mock-edited control cells were mixed with 5×10⁴ BDC2.5-CD4⁺ Teff and injected into 8-10 week-old male NSG mice (FIG. 198E). Recipients were monitored for up to 49 days for diabetes development. Consistent with previous reports, polyclonal tTreg were minimally effective in preventing T1D development, with only ˜20%- of mice remaining diabetes-free. Similarly, only limited protection was observed in recipients of polyclonal EngTregs. In contrast, nearly all mice (˜95%) receiving BDC2.5 islet specific-EngTregs or −tTreg remained diabetes-free (FIG. 198E). Taken together, these findings demonstrate a robust capacity for islet-specific EngTregs to prevent T1D development in vivo and, consistent to previous work using tTreg, they show that expression of an islet specific TCR markedly improves the potency of EngTregs.

### DISCUSSION

As described herein, the ability of antigen specific T cells derived from PBMC followed by FOXP3 editing to function in an antigen specific manner was demonstrated (30). While technically feasible, this method had several limitations in the context of autoimmunity: T cells specific for self-antigens are rare in the peripheral blood and expansion to numbers and cell purities likely to be required for therapeutic application are difficult and time consuming. Hence, as disclosed herein, efforts were focused on combining efficient delivery of islet specific TCRs derived from T1D subjects ((31) and unpublished data) with HDR-gene editing. This combined approach enabled a >10-fold expansion of antigen-specific EngTregs derived from the initial CD4⁺ T cells isolated from PBMC. Importantly, a suppressive capacity of islet-specific EngTregs under antigen-specific stimulation was demonstrated which had not been seen with either polyclonal-EngTregs or tTreg. Accordingly, the study disclosed herein in a murine model of T1D also showed that islet antigen-specific EngTregs blocked diabetes triggered by islet-specific Teff, while polyclonal EngTregs failed to limit disease progression.

It has been suggested that Treg expressing TCRs that recognize tissue-specific peptides may preferentially accumulate in target tissues, where they can be activated by these autoantigens and mediate bystander suppression (58). Mouse studies disclosed herein showed that islet-specific EngTregs predominantly localized in the pancreas following adoptive transfer and effectively suppressed diabetes triggered by islet-specific Teff. Given the possibility that polyclonal Treg can interfere with immune responses to pathogens, the ability to home to target tissues is likely critical for both efficient on-target immune suppression and for limiting the risk of impairing systemic immunity (8, 14). Further, in vitro data in human cells demonstrated that islet-specific EngTregs suppress bystander Teff with many different specificities. This breadth of bystander suppression is predicted to permit islet-specific EngTregs to locally suppress pathogenic Teff with multiple specificities including limiting Teff responses where the target autoantigens are unknown. Thus, the combination of the targeted homing and bystander suppressive capacity by EngTregs with islet-TCR likely provides a more efficient and safer strategy to treat and control autoimmune diabetes (59).

Functional studies of antigen specific human Treg is largely limited to in vitro suppression assays and it remains unclear whether these assays accurately predict in vivo function. While islet-specific EngTregs were demonstrated to mediate efficient suppressive activity on Teff in a model system utilizing Teff transduced with a relevant TCR, this system has limitations based upon testing Teff with a single specificity. Given the diversity of pathogenic autoreactive T cells in T1D, whether islet-specific EngTregs would also inhibit endogenous T1D-relevant Teff with a broad range of TCR specificities was investigated. The data provide a significant advance in this arena. Using a pool of antigen enriched, islet-specific Teff derived from T1D subjects (based upon stimulation with a broad panel of islet peptides across multiple antigens), it was demonstrated that the capacity to suppress polyclonal Teff populations using islet-specific EngTregs with single islet antigen specificity. These findings support the concept that islet-specific EngTregs can mediate antigen-specific and bystander suppression of autologous, islet-specific Teff present in T1D subjects.

Multiple mechanisms have been implicated in the suppression of CD4 T cells by Treg including modulation of costimulatory receptors on APC, production of soluble factors (such as generation of adenosine by conversion of ATP via CD39/CD73, IL-10, TGF-β and IL-35) and consumption of IL-2 (8, 46, 60). Here, mechanisms whereby islet specific EngTregs function were explored taking advantage of an ability to assess suppression in an antigen specific manner using autologous T1D subject-derived, CD4 T effectors enriched for specificity to islet antigens. Using this approach, it was demonstrated that, while IL-2 consumption is not a driver of suppression in this setting, EngTregs can function to down-modulate APC activation. Further, the transwell-based analyses showed a contribution of both contact-independent and -dependent suppressive activity. The data support an important role for soluble EngTreg secreted factors in Teff suppression. Further, the loss of suppression of Teff in the lower wells, when EngTregs suppressed co-cultured Teff in the upper wells, indicates that these soluble factors are likely consumed by Teff in closest proximity. The findings related to IL-2 consumption and the role of contact and non-contact suppression are consistent with some but not all studies of tTreg in the literature (23, 48, 52). Differences in the studies disclosed herein and those of others may be due to differences between murine and human Treg and/or the use of non-specific stimulation in the absence of APC, or use effectors with a single specificity as compared to use of polyclonal antigen-specific T cells in these studies (23, 48). Additionally, EngTregs have been derived from CD4 effector T cells and edited to constitutively express FOXP3 at relatively high levels and thus may have different functional characteristics than tTreg.

A difference in suppression between TCR with the same MHC-peptide restriction but with different functional avidities was shown. Studies utilizing CAR or TRuC receptors in Tregs indicate that the character of the signal can play a significant role in Treg function. (50, 61). Studies in murine models using Tg TCR have suggested that Treg with high functional avidity are more potent (62, 63). However, a role of low affinity Treg has also been shown in a polyclonal NOD model (64). In that study, low affinity Treg were able to compete with high affinity Treg, accumulate in sites of inflammation and the combined presence of both low and high avidity Tregs gave greater protection from diabetes (64). Studies of transduced human Treg are limited. Low affinity Class I restricted TCR transduced into Treg confer potent antigen-specific suppressive activity and impede expansion of high avidity CD8 T cells (65). A study of Treg expressing GAD 555-567 specific TCRs, 4.13 and R164 (low and high affinity respectively), demonstrated the capacity of each TCR to confer regulatory function. However, when R164 and 4.13 TCR Treg were compared for the capacity to suppress R164 T cells, 4.13 showed lower suppression, suggesting an advantage for the high avidity TCR (66). In the study disclosed herein, we employed a polyclonal islet-specific Teff pool and EngTreg expressing three alternative TCRs restricted to the same MHC-peptide complex was employed. In this setting, the TCR with lowest functional avidity yielded the greatest suppression. The findings herein may differ from previous studies due to a difference in the type of Teff, the culture conditions or differences in the mechanism(s) required for suppression by EngTreg.

In summary, described herein is an efficient strategy to generate antigen-specific EngTregs from primary CD4⁺ T cells via combining of FOXP3 HDR-editing and LV TCR transfer. It was shown that EngTregs expressing islet-TCRs suppressed both proliferation and cytokine production of antigen-specific and bystander effector Teff. Further, islet-specific EngTregs suppressed autologous pathogenic polyclonal T cells expanded from PBMC of TID patients. Consistent with these findings, adoptively transferred, islet-specific EngTregs selectively accumulated in the pancreas and prevented diabetes triggered by islet-specific Teff in vivo in recipient mice. Taken together, these findings strongly support the use of antigen-specific EngTregs in treatment of T1D and in other organ specific autoimmune or inflammatory disorders.

### MATERIALS AND METHODS

### Study Design

The objective of this study was to test whether durable, antigen-specific EngTregs could be generated using a gene editing approach combining FOXP3 homology directed repair editing and lentiviral TCR delivery. The ability of human islet specific EngTregs to suppress Teff proliferation and cytokine production in the presence of the cognate vs. irrelevant antigens were assessed in vitro. The ability of murine islet-specific EngTregs to traffic to the pancreas, prevent diabetes, and stably persist in vivo were assessed in a T1D mouse model using BDC2.5-CD4+ Teff to induce disease. Investigators were not blinded to the treatment. Figure legends list the sample size, number of biological replicates, number of independent experiments and statistical method.

### Primary Human T Cells

Human PBMCs were obtained from the Benaroya Research Institute (BRI) Registry and Repository were approved by BRI's Institutional Review Board (IRB #07109-588). Healthy control subjects had no personal or family history of autoimmune disease. Both healthy control and T1D subjects were HLA DRB1*0401.

### LV Transduction and Foxp3 Editing

CD4⁺ T cells were isolated from PBMC by magnetic bead CD4⁺ T cell isolation kit (Miltenyi) and cultured in RPMI 1640 media supplemented with 20% human serum and penicillin/streptomycin. T cells were activated with CD3/CD28 activator beads at a 1:1 bead to cell ratio and recombinant human IL-2, IL-7, and IL-15 at 50, 5, and 5 ng/ml, respectively on day 0. After 24 h activation, transduction with LV vectors encoding GAD65, IGRP, or PPI specific TCRs was performed by adding concentrated LV supernatant with polybrene at 10 g/ml. Beads were removed after 24 h incubation and cells were either rested for 16-24 h for editing or expanded in media with IL-2 (20 ng/ml) until day 14 or day 15 to be used as Teff cells. For Foxp3 editing, cells were transfected by electroporation with RNP complex combined with Cas9 and guide RNA and then transduced with AAV template. 20-24 h after editing, cells were expanded in media with IL-2 (100 ng/ml) until day 10 and islet-specific LNGFR⁺ EngTregs were enriched by LNGFR magnetic beads. LNGFR⁻ T cells also collected from the LNGFR⁺ cell enrichment to be used as controls in suppression assays.

### In Vitro Expansion and Tetramer Staining of Islet-Specific T Cells Derived from PBMC

For expanding islet-specific T cells by peptide stimulation, CD4⁺ T cells (CD4⁺CD25⁻) were isolated from PBMC and incubated with irradiated autologous CD4⁻CD25⁺ cells and a pool of islet-specific peptides (GAD65₁₁₃₋₁₃₂, GAD65₂₆₅₋₂₈₄, GAD65₂₇₃₋₂₉₂, GAD65₃₀₅₋₃₂₄, GAD65₅₅₃₋₅₇₂, IGRP₁₇₋₃₆, IGRP₂₄₁₋₂₆₀, IGRP₃₀₅₋₃₂₄, and PPI₇₆₋₉₀) at 5 g/ml. After 7 days of incubation, part of the T cells were harvested as day 7 islet-specific Teff and remaining cells were expanded in media with IL-2 at 20 ng/ml. IL-2 was added in 2-3 days of interval and cells were collected at day 14 as day 14 islet-specific Teff. In order to check population of expanded islet-specific T cells, day 14 Teff were incubated with PE-tagged tetramer for 1 h and followed by surface staining. For mechanistic experiments (Transwell suppression assay, IL-2 consumption, and TCR avidity), polyclonal islet-specific T cells were expanded with a pool of 9 islet-specific peptides (GAD65₁₁₃₋₁₃₂, GAD₂₆₅₋₂₈₄, GAD65₂₇₃₋₂₉₂, GAD65₃₀₅₋₃₂₄, GAD65₅₅₃₋₅₇₂, IGRP₁₇₋₃₆, IGRP₂₄₁₋₂₆₀, PPI₇₆₋₉₀, ZNT8₂₆₆₋₂₈₅) excluding IGRP₃₀₅-324 that is specific for T1D2 EngTregs to measure bystander suppression.

### In Vitro Suppression Assay Using CD3/CD28 Beads

2×10⁴ Teff were cultured alone or co-cultured with EngTregs or LNGFR⁻ T cells at 1:1 ratio in the presence of CD3/CD28 beads in 96 well plate. 1:28 or 1:32 of beads to Teff ratio was used for 3- or 4-day culture, respectively. Teff and EngTregs or LNGFR⁻ T cells were labeled with Cell Trace Violet (Invitrogen) and EF670 (Thermo Fisher), respectively, before the co-culture. Dilution of Cell Trace Violet (CTV) was measured as proliferation of Teff and % suppression was calculated as (a-b)/a×100 where a is the percentage Teff proliferation in the absence of Treg and b is the percentage of Teff proliferation in the presence of Treg.

### Antigen-Specific Suppression Assay

Autologous PBMC were irradiated at 5,000 rad and used as APC in the suppression assay using Teff transduced with TCR. CTV-labeled Teff were co-cultured with EF670-labeled EngTregs or islet-specific LNGFR⁻ T cells at 1:1 ratio in the presence of APC and DMSO or relevant peptide. Cells were incubated for 4 days and stained for measuring Teff proliferation. For measuring intracellular cytokines produced by Teff, cells were cultured for 3 days and incubated with Brefeldin A for another 4 h, followed by intracellular staining.

### Antigen-Specific Suppression Assay Using Polyclonal Islet-Specific Teff Derived from T1D PBMC

CD14⁺ cells, CD4⁺CD25⁻, and CD4⁻CD25+ cells were isolated from 60 million PBMC of donors with T1D. CD14⁺ cells isolated using CD14 microbeads (Miltenyi) were cultured in media supplemented with GM-CSF and TL-4 at 800 U/ml and 1,000 U/ml, respectively, for 7 days to differentiate into monocyte-derived DC (mDC). CD4⁺CD25⁻ cells were divided, some used to generate EngTregs and the rest were used for in vitro expansion of polyclonal islet-specific Teff using 9 islet-specific peptides and irradiated autologous CD4⁻ CD25⁺ cells as described above. For the suppression assay, polyclonal islet-specific Teff harvested at day 7 or day 14 were co-cultured with or without poly EngTregs, T1D2 EngTregs, 4.13 EngTregs, or LNGFR⁻ T cells in the presence of autologous mDC and DMSO or 9 islet-specific peptides for 4 days. EngTregs/LNGFR⁻ T cells and polyclonal islet-specific Teff were labeled with EF670 and CTV, respectively, before the co-culture.

### Transwell Suppression Assay

mDC were pre-incubated with a pool of 10 islet-specific peptides (GAD65₁₁₃₋₁₃₂, GAD265-284, GAD65₂₇₃₋₂₉₂, GAD65₃₀₅₋₃₂₄, GAD65₅₅₃₋₅₇₂, IGRP₁₇₋₃₆, IGRP₂₄₁₋₂₆₀, IGRP₃₀₅₋₃₂₄, PPI₇₆₋₉₀, ZNT8₂₆₆₋₂₈₅) for 1 hour, washed, and plated in both upper and lower chambers of 96 well transwell plate with pore size 0.4 M (Corning). Polyclonal islet-specific Teff generated by stimulation with 9 islet-peptides (GAD65₁₁₃₋₁₃₂, GAD₂₆₅₋₂₈₄, GAD65₂₇₃₋₂₉₂, GAD65₃₀₅₋₃₂₄, GAD65₅₅₃₋₅₇₂, IGRP₁₇₋₃₆, IGRP₂₄₁₋₂₆₀, PPI₇₆₋₉₀, ZNT8₂₆₆₋₂₈₅) and T1D2 EngTregs were plated, where indicated. Cell populations being assessed for regulatory capacity were cultured in the upper chamber. Polyclonal islet Teff and T1D2 EngTregs were labeled with CTV and EF670, respectively, before the co-culture. After 4 days in culture, cells from both chambers were harvested and stained for FACS analysis. CTV dilution was measured to assess Teff proliferation.

### Suppression of APC Maturation Assay

CD14+ monocytes were isolated from PBMC and were cultured in the presence of GM-CSF and IL-4 for 7 days to differentiate into mDC. In the last 16-18 hours of culture, IFN-γ and CL075 were added for maturation. Matured mDC were co-cultured for 2 days with autologous CTV-labeled T1D2 EngTregs (or LNGFR⁻ T cells) at 1:2 ratio of mDC to EngTregs/LNGFR−T cells in the presence of IGRP³⁰⁵⁻³²⁴ peptide. Cells were harvested and analyzed for surface marker expression (CD86 or CD80) on DC. MFI of CD86/CD80 on mDCs were normalized by MFI of mDC only condition.

### Mice

NOD and NOD BDC2.5 mice were purchased from The Jackson Laboratory then bred and maintained at the Seattle Children's Research Institute (SCRI) SPF facility to produce the mice used in experiments here. Experimental NSG mice were purchased from The Jackson Laboratory, and acclimated at SCRI for 1-2 weeks before experiments. Experiments, breeding, and handling of mice were conducted in accordance with the National Institutes of Health's Guide for the Care and Use of Laboratory Animals using protocols approved by the Institutional Animal Care and Use Committee at the SCRI.

### Primary Mouse T Cell Isolation, Culture, Editing and Enrichment

To obtain CD4⁺ T cells for gene editing, mouse lymphocytes from spleen and lymph nodes of 8-12 weeks old NOD BDC2.5 and NOD mice were isolated and combined. CD4⁺ T cells were purified from lymphocytes by negative selection using EasySep mouse CD4⁺ T Cell Enrichment Kit (STEMCELL Technologies), then activated using mouse specific anti −CD3/CD28 coated beads (Gibco) for ˜40 hrs in a RPMI media containing 20% FBS (Omega Scientific Inc., Catalog #FB-11), HEPES, Glutamax, β-mercaptoethanol and 50 ng/mL mouse IL-2 (Peprotech). After activation, cells were separated from beads and further cultured for ˜10 hours in media then washed twice in PBS and resuspend in Buffer R (Neon kit, Invitrogen) at 25×10⁶ cells/mL. RNP was prepared in Buffer R by mixing 20 pmol of Cas9 (IDT) with 50 pmol of mouse Foxp3 specific gRNA for 25 min at room temperature. Delivery of RNP into mouse cells was achieved by electroporation (1550V, 10 ms and 3 pulses) using Neon system (Thermo Fisher Scientific) followed by incubation with AAV5 containing donor template with homology sequence to mouse Foxp3 for ˜20-24 hours at 37° C. Cells were replenished (two-fold dilution) with fresh media containing IL-2 and transferred to a new tissue culture dish for another ˜16 hours before final analysis and enrichment. Two days post editing, edited cells were collected, counted and stained for biotinylated anti-LNGFR antibody (Miltenyi Biotech) and enriched using anti-biotin microbeads (Miltenyi Biotech) as described (67).

### Isolation and Enrichment of Antigen Specific Teff and tTreg

Murine effector CD4⁺ T cells used experimentally were CD4⁺ CD25⁻, and were enriched via negative selection of CD4 and CD25 (Miltenyi Biotec) from combined single cell suspensions obtained from spleen and lymph nodes of NOD BDC2.5 mice. Murine CD4⁺ Teff were freshly prepared for each experiment. CD4⁺ CD25⁺ tTreg from antigen-specific NOD BDC2.5 and polyclonal NOD mice were enriched using a murine Treg enrichment kit (Miltenyi Biotec) according to the manufacturer's instructions. Enriched (≥90%) tTreg were activated to match EngTregs activation status and timeline, in the same media used to culture EngTregs. Activated tTreg were immunophenotyped then cryopreserved in LN₂. Prior to injection, tTreg were thawed and rested in IL-2 containing media overnight. Viability and CD4⁺ CD25⁺ FOXP3⁺ phenotype was confirmed by flow cytometry prior to injection.

### Diabetes Induction and Monitoring

8-10 week-old male NSG mice were pre-screened for normal blood glucose values before enrolling in diabetes prevention studies. Mice were injected with 5×10⁴ islet specific Teff cells in combination with 5×10⁴ (1:1) mock-edited control, tTreg or EngTregs; in some conditions 1×10⁵ (1:2 Teff:Treg) Treg were injected. Cells were delivered via the retro-orbital sinus. Diabetes was monitored by peripheral blood sampling using a Bayer Contour Blood Glucose Monitor System (Bayer). Mice with blood glucose >250 mg/dL twice within 24 hrs or exceeding 400 mg/dL were considered diabetic and were euthanized

### Statistical Analysis

GraphPad Prism version 8 was used to conduct all statistical analyses. Specifics of the statistical tests used are indicated in each figure legend; no outliers were excluded.

All references cited herein, including those listed below are expressly incorporated by reference herein in their entireties.

### REFERENCES AND NOTES

1. M. Willberg, A. Cooke, Immune mechanisms in type 1 diabetes. Trends in Immunology 34, 583-591 (2013).
2. A. Pugliese, Autoreactive T cells in type 1 diabetes. J Clin Invest 127, 2881-2891 (2017).
3. C. M. Hull, M. Peakman, T. I. M. Tree, Regulatory T cell dysfunction in type 1 diabetes: what's broken and how can we fix it? Diabetologia 60, 1839-1850 (2017).
4. A. Ferraro, C. Socci, A. Stabilini, A. Valle, P. Monti, L. Piemonti, R. Nano, S. Olek, P. Maffi, M. Scavini, A. Secchi, C. Staudacher, E. Bonifacio, M. Battaglia, Expansion of Th17 cells and functional defects in T regulatory cells are key features of the pancreatic lymph nodes in patients with type 1 diabetes. Diabetes 60, 2903-2913 (2011).
5. S. Lindley, C. M. Dayan, A. Bishop, B. O. Roep, M. Peakman, T. I. Tree, Defective suppressor function in CD4(+)CD25(+) T-cells from patients with type 1 diabetes. Diabetes 54, 92-99 (2005).
6. J. A. Bluestone, J. H. Buckner, M. Fitch, S. E. Gitelman, S. Gupta, M. K. Hellerstein, K. C. Herold, A. Lares, M. R. Lee, K. Li, W. Liu, S. A. Long, L. M. Masiello, V. Nguyen, A. L. Putnam, M. Rieck, P. H. Sayre, Q. Tang, Type 1 diabetes immunotherapy using polyclonal regulatory T cells. Sci Transl Med 7, 315ra189 (2015).
7. J. H. Esensten, Y. D. Muller, J. A. Bluestone, Q. Tang, Regulatory T-cell therapy for autoimmune and autoinflammatory diseases: The next frontier. The Journal of allergy and clinical immunology 142, 1710-1718 (2018).
8. C. Raffin, L. T. Vo, J. A. Bluestone, T(reg) cell-based therapies: challenges and perspectives. Nat Rev Immunol 20, 158-172 (2020).
9. Q. Tang, K. J. Henriksen, M. Bi, E. B. Finger, G. Szot, J. Ye, E. L. Masteller, H. McDevitt, M. Bonyhadi, J. A. Bluestone, In vitro-expanded antigen-specific regulatory T cells suppress autoimmune diabetes. The Journal of experimental medicine 199, 1455-1465 (2004).
10. K. V. Tarbell, S. Yamazaki, K. Olson, P. Toy, R. M. Steinman, CD25+ CD4+ T cells, expanded with dendritic cells presenting a single autoantigenic peptide, suppress autoimmune diabetes. The Journal of experimental medicine 199, 1467-1477 (2004).
11. L. A. Stephens, K. H. Malpass, S. M. Anderton, Curing CNS autoimmune disease with myelin-reactive Foxp3+ Treg. Eur J Immunol 39, 1108-1117 (2009).
12. P. Zhou, R. Borojevic, C. Streutker, D. Snider, H. Liang, K. Croitoru, Expression of dual TCR on DO11.10 T cells allows for ovalbumin-induced oral tolerance to prevent T cell-mediated colitis directed against unrelated enteric bacterial antigens. J Immunol 172, 1515-1523 (2004).
13. K. Fujio, A. Okamoto, Y. Araki, H. Shoda, H. Tahara, N. H. Tsuno, K. Takahashi, T. Kitamura, K. Yamamoto, Gene therapy of arthritis with TCR isolated from the inflamed paw. J Immunol 177, 8140-8147 (2006).
14. G. P. Wright, C. A. Notley, S. A. Xue, G. M. Bendle, A. Holler, T. N. Schumacher, M. R. Ehrenstein, H. J. Stauss, Adoptive therapy with redirected primary regulatory T cells results in antigen-specific suppression of arthritis. Proc Natl Acad Sci USA 106, 19078-19083 (2009).
15. J. Y. Tsang, Y. Tanriver, S. Jiang, S. A. Xue, K. Ratnasothy, D. Chen, H. J. Stauss, R. P. Bucy, G. Lombardi, R. Lechler, Conferring indirect all of specificity on CD4+CD25+ Tregs by TCR gene transfer favors transplantation tolerance in mice. J Clin Invest 118, 3619-3628 (2008).
16. E. L. Masteller, M. R. Warner, Q. Tang, K. V. Tarbell, H. McDevitt, J. A. Bluestone, Expansion of functional endogenous antigen-specific CD4+CD25+ regulatory T cells from nonobese diabetic mice. J Immunol 175, 3053-3059 (2005).
17. K. V. Tarbell, L. Petit, X. Zuo, P. Toy, X. Luo, A. Mqadmi, H. Yang, M. Suthanthiran, S. Mojsov, R. M. Steinman, Dendritic cell-expanded, islet-specific CD4+ CD25+ CD62L+ regulatory T cells restore normoglycemia in diabetic NOD mice. The Journal of experimental medicine 204, 191-201 (2007).
18. C. G. Brunstein, J. S. Miller, Q. Cao, D. H. McKenna, K. L. Hippen, J. Curtsinger, T. Defor, B. L. Levine, C. H. June, P. Rubinstein, P. B. McGlave, B. R. Blazar, J. E. Wagner, Infusion of ex vivo expanded T regulatory cells in adults transplanted with umbilical cord blood: safety profile and detection kinetics. Blood 117, 1061-1070 (2011).
19. S. Sakaguchi, T. Yamaguchi, T. Nomura, M. Ono, Regulatory T cells and immune tolerance. Cell 133, 775-787 (2008).
20. K. L. Hippen, S. C. Merkel, D. K. Schirm, C. M. Sieben, D. Sumstad, D. M. Kadidlo, D. H. McKenna, J. S. Bromberg, B. L. Levine, J. L. Riley, C. H. June, P. Scheinberg, D. C. Douek, J. S. Miller, J. E. Wagner, B. R. Blazar, Massive ex vivo expansion of human natural regulatory T cells (T(regs)) with minimal loss of in vivo functional activity. Sci Transl Med 3, 83ra41 (2011).
21. C. Baecher-Allan, J. A. Brown, G. J. Freeman, D. A. Hafler, CD4+CD25 high regulatory cells in human peripheral blood. J Immunol 167, 1245-1253 (2001).
22. J. A. Bluestone, Q. Tang, T(reg) cells-the next frontier of cell therapy. Science 362, 154-155 (2018).
23. Y. C. Kim, A. H. Zhang, J. Yoon, W. E. Culp, J. R. Lees, K. W. Wucherpfennig, D. W. Scott, Engineered MBP-specific human Tregs ameliorate MOG-induced EAE through IL-2-triggered inhibition of effector T cells. J Autoimmun 92, 77-86 (2018).
24. Z. Zhang, W. Zhang, J. Guo, Q. Gu, X. Zhu, X. Zhou, Activation and Functional Specialization of Regulatory T Cells Lead to the Generation of Foxp3 Instability. J Immunol 198, 2612-2625 (2017).
25. D. V. Sawant, D. A. Vignali, Once a Treg, always a Treg? Immunol Rev 259, 173-191 (2014).
26. N. Komatsu, M. E. Mariotti-Ferrandiz, Y. Wang, B. Malissen, H. Waldmann, S. Hori, Heterogeneity of natural Foxp3+ T cells: a committed regulatory T-cell lineage and an uncommitted minor population retaining plasticity. Proc Natl Acad Sci USA 106, 1903-1908 (2009).
27. X. Zhou, S. L. Bailey-Bucktrout, L. T. Jeker, C. Penaranda, M. Martinez-Llordella, M. Ashby, M. Nakayama, W. Rosenthal, J. A. Bluestone, Instability of the transcription factor Foxp3 leads to the generation of pathogenic memory T cells in vivo. Nat Immunol 10, 1000-1007 (2009).
28. S. L. Bailey-Bucktrout, M. Martinez-Llordella, X. Zhou, B. Anthony, W. Rosenthal, H. Luche, H. J. Fehling, J. A. Bluestone, Self-antigen-driven activation induces instability of regulatory T cells during an inflammatory autoimmune response. Immunity 39, 949-962 (2013).
29. N. Komatsu, K. Okamoto, S. Sawa, T. Nakashima, M. Oh-Hora, T. Kodama, S. Tanaka, J. A. Bluestone, H. Takayanagi, Pathogenic conversion of Foxp3(+) T cells into TH17 cells in autoimmune arthritis. Nat. Med. 20, 62-68 (2014).
30. Y. Honaker, N. Hubbard, Y. Xiang, L. Fisher, D. Hagin, K. Sommer, Y. Song, S. J. Yang, C. Lopez, T. Tappen, E. M. Dam, I. Khan, M. Hale, J. H. Buckner, A. M. Scharenberg, T. R. Torgerson, D. J. Rawlings, Gene editing to induce FOXP3 expression in human CD4(+) T cells leads to a stable regulatory phenotype and function. Sci Transl Med 12, (2020).
31. K. Cerosaletti, F. Barahmand-Pour-Whitman, J. Yang, H. A. DeBerg, M. J. Dufort, S. A. Murray, E. Israelsson, C. Speake, V. H. Gersuk, J. A. Eddy, H. Reijonen, C. J. Greenbaum, W. W. Kwok, E. Wambre, M. Prlic, R. Gottardo, G. T. Nepom, P. S. Linsley, Single-Cell RNA Sequencing Reveals Expanded Clones of Islet Antigen-Reactive CD4(+) T Cells in Peripheral Blood of Subjects with Type 1 Diabetes. J Immunol 199, 323-335 (2017).
32. B. Fehse, A. Uhde, N. Fehse, H. G. Eckert, J. Clausen, R. Ruger, S. Koch, W. Ostertag, A. R. Zander, M. Stockschlader, Selective immunoaffinity-based enrichment of CD34+ cells transduced with retroviral vectors containing an intracytoplasmatically truncated version of the human low-affinity nerve growth factor receptor (deltaLNGFR) gene. Hum Gene Ther 8, 1815-1824 (1997).
33. B. D. Singer, L. S. King, F. R. D'Alessio, Regulatory T cells as immunotherapy. Frontiers in immunology 5, 46 (2014).
34. A. E. Herman, G. J. Freeman, D. Mathis, C. Benoist, CD4+CD25+ T regulatory cells dependent on ICOS promote regulation of effector cells in the prediabetic lesion. The Journal of experimental medicine 199, 1479-1489 (2004).
35. K. Wing, Y. Onishi, P. Prieto-Martin, T. Yamaguchi, M. Miyara, Z. Fehervari, T. Nomura, S. Sakaguchi, CTLA-4 control over Foxp3+ regulatory T cell function. Science 322, 271-275 (2008).
36. D. K. Sojka, A. Hughson, T. L. Sukiennicki, D. J. Fowell, Early kinetic window of target T cell susceptibility to CD25+ regulatory T cell activity. J Immunol 175, 7274-7280 (2005).
37. N. Oberle, N. Eberhardt, C. S. Falk, P. H. Krammer, E. Suri-Payer, Rapid suppression of cytokine transcription in human CD4+CD25 T cells by CD4+Foxp3+ regulatory T cells: independence of IL-2 consumption, TGF-beta, and various inhibitors of TCR signaling. J Immunol 179, 3578-3587 (2007).
38. A. Schmidt, N. Oberle, E. M. Weiss, D. Vobis, S. Frischbutter, R. Baumgrass, C. S. Falk, M. Haag, B. Brugger, H. Lin, G. W. Mayr, P. Reichardt, M. Gunzer, E. Suri-Payer, P. H. Krammer, Human regulatory T cells rapidly suppress T cell receptor-induced Ca(2+), NF-κB, and NFAT signaling in conventional T cells. Science signaling 4, ra90 (2011).
39. A. Schmidt, N. Oberle, P. H. Krammer, Molecular mechanisms of treg-mediated T cell suppression. Frontiers in immunology 3, 51 (2012).
40. E. M. Shevach, Foxp3(+) T Regulatory Cells: Still Many Unanswered Questions-A Perspective After 20 Years of Study. Frontiers in immunology 9, 1048 (2018).
41. N. A. Danke, J. Yang, C. Greenbaum, W. W. Kwok, Comparative study of GAD65-specific CD4+ T cells in healthy and type 1 diabetic subjects. J. Autoimmun. 25, 303-311 (2005).
42. J. Yang, N. A. Danke, D. Berger, S. Reichstetter, H. Reijonen, C. Greenbaum, C. Pihoker, E. A. James, W. W. Kwok, Islet-specific glucose-6-phosphatase catalytic subunit-related protein-reactive CD4+ T cells in human subjects. J. Immunol. 176, 2781-2789 (2006).
43. J. Yang, N. Danke, M. Roti, L. Huston, C. Greenbaum, C. Pihoker, E. James, W. W. Kwok, CD4+ T cells from type 1 diabetic and healthy subjects exhibit different thresholds of activation to a naturally processed proinsulin epitope. J. Autoimmun. 31, 30-41 (2008).
44. S. A. Long, M. R. Walker, M. Rieck, E. James, W. W. Kwok, S. Sanda, C. Pihoker, C. Greenbaum, G. T. Nepom, J. H. Buckner, Functional islet-specific Treg can be generated from CD4+CD25− T cells of healthy and type 1 diabetic subjects. Eur. J. Immunol. 39, 612-620 (2009).
45. J. Yang, E. A. James, S. Sanda, C. Greenbaum, W. W. Kwok, CD4+ T cells recognize diverse epitopes within GAD65: implications for repertoire development and diabetes monitoring. Immunology 138, 269-279 (2013).
46. D. A. Vignali, L. W. Collison, C. J. Workman, How regulatory T cells work. Nat. Rev. Immunol. 8, 523-532 (2008).
47. A. M. Thornton, E. M. Shevach, CD4+CD25+ immunoregulatory T cells suppress polyclonal T cell activation in vitro by inhibiting interleukin 2 production. The Journal of experimental medicine 188, 287-296 (1998).
48. L. W. Collison, M. R. Pillai, V. Chaturvedi, D. A. Vignali, Regulatory T cell suppression is potentiated by target T cells in a cell contact, IL-35- and IL-10-dependent manner. J. Immunol. 182, 6121-6128 (2009).
49. Y. Onishi, Z. Fehervari, T. Yamaguchi, S. Sakaguchi, Foxp3+ natural regulatory T cells preferentially form aggregates on dendritic cells in vitro and actively inhibit their maturation. Proc Natl Acad Sci USA 105, 10113-10118 (2008).
50. N. A. J. Dawson, I. Rosado-Sinchez, G. E. Novakovsky, V. C. W. Fung, Q. Huang, E. McIver, G. Sun, J. Gillies, M. Speck, P. C. Orban, M. Mojibian, M. K. Levings, Functional effects of chimeric antigen receptor co-receptor signaling domains in human regulatory T cells. Sci Transl Med 12, (2020).
51. P. Pandiyan, L. Zheng, S. Ishihara, J. Reed, M. J. Lenardo, CD4+CD25+Foxp3+ regulatory T cells induce cytokine deprivation-mediated apoptosis of effector CD4+ T cells. Nat Immunol 8, 1353-1362 (2007).
52. D. Q. Tran, D. D. Glass, G. Uzel, D. A. Darnell, C. Spalding, S. M. Holland, E. M. Shevach, Analysis of adhesion molecules, target cells, and role of IL-2 in human FOXP3+ regulatory T cell suppressor function. J Immunol 182, 2929-2938 (2009).
53. J. D. Katz, B. Wang, K. Haskins, C. Benoist, D. Mathis, Following a diabetogenic T cell from genesis through pathogenesis. Cell 74, 1089-1100 (1993).
54. M. S. Anderson, J. A. Bluestone, The NOD mouse: a model of immune dysregulation. Annu Rev Immunol 23, 447-485 (2005).
55. K. Haskins, D. Wegmann, Diabetogenic T-cell clones. Diabetes 45, 1299-1305 (1996).
56. K. Haskins, M. McDuffie, Acceleration of diabetes in young NOD mice with a CD4+ islet-specific T cell clone. Science 249, 1433-1436 (1990).
57. M. Presa, Y. G. Chen, A. E. Grier, E. H. Leiter, M. A. Brehm, D. L. Greiner, L. D. Shultz, D. V. Serreze, The Presence and Preferential Activation of Regulatory T Cells Diminish Adoptive Transfer of Autoimmune Diabetes by Polyclonal Nonobese Diabetic (NOD) T Cell Effectors into NSG versus NOD-scid Mice. J Immunol 195, 3011-3019 (2015).
58. Q. Tang, J. A. Bluestone, The Foxp3+ regulatory T cell: a jack of all trades, master of regulation. Nat. Immunol. 9, 239-244 (2008).
59. J. L. McGovern, G. P. Wright, H. J. Stauss, Engineering Specificity and Function of Therapeutic Regulatory T Cells. Frontiers in immunology 8, 1517 (2017).
60. T. Maj, W. Wang, J. Crespo, H. Zhang, W. Wang, S. Wei, L. Zhao, L. Vatan, I. Shao, W. Szeliga, C. Lyssiotis, J. R. Liu, I. Kryczek, W. Zou, Oxidative stress controls regulatory T cell apoptosis and suppressor activity and PD-L1-blockade resistance in tumor. Nat Immunol 18, 1332-1341 (2017).
61. J. Rana, D. J. Perry, S. R. P. Kumar, M. Munoz-Melero, R. Saboungi, T. M. Brusko, M. Biswas, CAR- and TRuC-redirected regulatory T cells differ in capacity to control adaptive immunity to FVIII. Molecular therapy: the journal of the American Society of Gene Therapy 29, 2660-2676 (2021).
62. J. Y. Tsang, K. Ratnasothy, D. Li, Y. Chen, R. P. Bucy, K. F. Lau, L. Smyth, G. Lombardi, R. Lechler, P. K. Tam, The potency of allospecific Tregs cells appears to correlate with T cell receptor functional avidity. Am J Transplant 11, 1610-1620 (2011).
63. M. Bettini, L. Blanchfield, A. Castellaw, Q. Zhang, M. Nakayama, M. P. Smeltzer, H. Zhang, K. A. Hogquist, B. D. Evavold, D. A. Vignali, TCR affinity and tolerance mechanisms converge to shape T cell diabetogenic potential. J Immunol 193, 571-579 (2014).
64. M. L. Sprouse, I. Shevchenko, M. A. Scavuzzo, F. Joseph, T. Lee, S. Blum, M. Borowiak, M. L. Bettini, M. Bettini, Cutting Edge: Low-Affinity TCRs Support Regulatory T Cell Function in Autoimmunity. J Immunol 200, 909-914 (2018).
65. G. Plesa, L. Zheng, A. Medvec, C. B. Wilson, C. Robles-Oteiza, N. Liddy, A. D. Bennett, J. Gavarret, A. Vuidepot, Y. Zhao, B. R. Blazar, B. K. Jakobsen, J. L. Riley, TCR affinity and specificity requirements for human regulatory T-cell function. Blood 119, 3420-3430 (2012).
66. W. I. Yeh, H. R. Seay, B. Newby, A. L. Posgai, F. B. Moniz, A. Michels, C. E. Mathews, J. A. Bluestone, T. M. Brusko, Avidity and Bystander Suppressive Capacity of Human Regulatory T Cells Expressing De Novo Autoreactive T-Cell Receptors in Type 1 Diabetes. Frontiers in immunology 8, 1313 (2017).
67. R. Gastpar, C. Gross, L. Rossbacher, J. Ellwart, J. Riegger, G. Multhoff, The cell surface-localized heat shock protein 70 epitope TKD induces migration and cytolytic activity selectively in human NK cells. J Immunol 172, 972-980 (2004).

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1.-54. (canceled)

55. An engineered regulatory T (Treg) cell comprising:
(i) a first polynucleotide in a genome of the cell, the first polynucleotide comprising a first promoter operably linked to a nucleotide sequence encoding a first chemically inducible signaling complex (CISC) component, the first CISC component comprising:
(a) a first extracellular binding domain;
(b) a first transmembrane domain; and
(c) a first cytoplasmic domain; and
(ii) a second polynucleotide in a TRAC locus of the genome, the second polynucleotide comprising second promoter operably linked to a nucleotide sequence encoding a second CISC component, the second CISC component comprising:
(a) a second extracellular binding domain;
(b) a second transmembrane domain; and
(c) a second cytoplasmic domain,
wherein dimerization of the first and second CISC components in the presence of a ligand induces a signal transduction event and proliferation of the engineered Treg cell,
wherein the first promoter is operably linked to a nucleotide sequence encoding FOXP3,
wherein:
(iii)(a)(1) the second promoter is operably linked to a nucleotide sequence encoding a chimeric antigen receptor (CAR);
(iii)(a)(2) the second promoter is operably linked to (A) a nucleotide sequence encoding a T cell receptor (TCR) alpha (TCRα) protein, and (B) a nucleotide sequence encoding a TCR beta (TCRβ) protein; or
(iii)(b) the second promoter is operably linked to (A) a nucleotide sequence encoding a TCRβ protein, and (B) a first portion of a TCRα protein, the first portion of the TCRα protein comprising a TCRα variable region and a TCRα joining region, wherein the nucleotide sequence encoding the first portion of the TCRα protein is in-frame with an endogenous nucleotide sequence encoding a second portion of the TCRα protein, the second portion comprising at least a portion of a TCRα constant region.

56. The engineered Treg cell of claim 55, wherein the second promoter is operably linked to the nucleotide sequence encoding the CAR.

57. The engineered Treg cell of claim 56, wherein the engineered Treg cell is a CD8+ Treg cell.

58. The engineered Treg cell of claim 55, wherein the engineered Treg cell is a CD4+ Treg cell.

59. The engineered Treg cell of claim 58, wherein the second promoter is operably linked to (A) the nucleotide sequence encoding the TCRα protein, and (B) the nucleotide sequence encoding the TCRβ protein.

60. The engineered Treg cell of claim 58, wherein the second promoter is operably linked to (A) the nucleotide sequence encoding the TCR protein, and (B) the first portion of the TCRα protein comprising the TCRα variable region and the TCRα joining region, wherein the nucleotide sequence encoding the first portion of the TCRα protein is in-frame with the endogenous nucleotide sequence encoding the second portion of the TCRα protein.

61. The engineered Treg cell of claim 55, wherein:
(i)(a) the first CISC component comprises, in N-to-C-terminal order, an FK506-binding protein (FKBP) domain, an IL-2 receptor gamma (IL-2Rγ) transmembrane domain, and an IL-2Rγ cytoplasmic domain, and
(i)(b) the second CISC component comprises, in N-to-C-terminal order, an FKBP-rapamycin-binding (FRB) domain, an IL-2 receptor beta (IL-2Rβ) transmembrane domain, and an IL-2Rβ cytoplasmic domain; or
(ii)(a) the first CISC component comprises, in N-to-C-terminal order, an FKBP-rapamycin-binding (FRB) domain, an IL-2 receptor beta (IL-2Rβ) transmembrane domain, and an IL-2Rβ cytoplasmic domain, and
(ii)(b) the second CISC component comprises, in N-to-C-terminal order, an FK506-binding protein (FKBP) domain, an IL-2 receptor gamma (IL-2Rγ) transmembrane domain, and an IL-2Rγ cytoplasmic domain.

62. The engineered Treg cell of claim 55, wherein the first promoter is operably linked to a first coding exon of an endogenous FOXP3 gene in the Treg cell, wherein the first promoter is downstream from a Treg-specific demethylated region (TSDR) in the endogenous FOXP3 gene.

63. The engineered Treg cell of claim 55, wherein the nucleotide sequence encoding FOXP3 is a FOXP3 cDNA sequence.

64. The engineered Treg cell of claim 55, wherein the first promoter is a constitutive promoter, and wherein the second promoter is a constitutive promoter.

65. The engineered Treg cell of claim 55, wherein the first promoter is an MND promoter, and wherein the second promoter is an MND promoter.

66. A pharmaceutical composition comprising the engineered Treg cell of claim 55, and a pharmaceutically acceptable excipient.

67. A method of producing a genetically engineered regulatory T (Treg) cell, the method comprising contacting a cell with:
(i) a first polynucleotide comprising a first promoter operably linked to a nucleotide sequence encoding a first CISC component, the first CISC component comprising:
(a) a first extracellular binding domain;
(b) a first transmembrane domain; and
(c) a first cytoplasmic domain;
and
(ii) a second polynucleotide comprising a second promoter operably linked to a nucleotide sequence encoding a second CISC component, the second CISC component comprising:
(a) a second extracellular binding domain;
(b) a second transmembrane domain; and
(c) a second cytoplasmic domain,
wherein dimerization of the first and second CISC components in the presence of a ligand induces a signal transduction event and proliferation of the cell,
wherein the first polynucleotide is inserted into a FOXP3 locus in a genome of the cell, and wherein, after insertion into the FOXP3 locus, the first promoter is operably linked to a nucleotide sequence encoding FOXP3,
wherein the second polynucleotide is inserted into a TRAC locus in the genome, and wherein, after insertion of the second polynucleotide into the TRAC locus:
(iii)(a)(1) the second promoter is operably linked to a nucleotide sequence encoding a chimeric antigen receptor (CAR);
(iii)(a)(2) the second promoter is operably linked to (A) a nucleotide sequence encoding a T cell receptor (TCR) alpha (TCRα) protein, and (B) a nucleotide sequence encoding a TCR beta (TCRβ) protein; or
(iii)(b) the second promoter is operably linked to (A) a nucleotide sequence encoding a TCRβ protein, and (B) a first portion of a TCRα protein, the first portion of the TCRα protein comprising a TCRα variable region and a TCRα joining region, wherein the nucleotide sequence encoding the first portion of the TCRα protein is in-frame with an endogenous nucleotide sequence encoding a second portion of the TCRα protein, the second portion comprising at least a portion of a TCRα constant region.

68. The method of claim 67, further comprising contacting the cell with:
(i) a first nuclease that targets the FOXP3 locus, or a nucleic acid encoding the first nuclease; and
(ii) a second nuclease that targets the TRAC locus, or a nucleic acid encoding the second nuclease.

69. The method of claim 67, further comprising contacting the cell with:
(i) an RNA-guided nuclease;
(ii) a first guide RNA (gRNA) comprising a spacer sequence that targets the FOXP3 locus or a nucleic acid encoding the first gRNA; and
(iii) a second gRNA comprising a spacer sequence that targets the TRAC locus.

70. The method of claim 67, further comprising contacting the cell with the ligand.

71. A method of treating, ameliorating, or inhibiting a disorder in a subject in need thereof, the method comprising administering the engineered Treg cell of claim 55 to a subject in need thereof.

72. The method of claim 71, wherein the disorder is selected from the group consisting of autoimmune disease, inflammatory disease, alloimmune disease, and allergic disease.

73. The method of claim 71, wherein the engineered Treg cell is autologous to the subject.

74. The method of claim 71, wherein the engineered Treg cell is allogeneic to the subject.
